(19) United States
(12) Patent Application Publication
(10) Pub. No.: US 2023/0087118 A1
(43) Pub. Date: Mar. 23, 2023

(54) IMIDAZO[1,2-a]PYRIDINYL DERIVATIVES AND THEIR USE IN THE TREATMENT OF DISEASE

(71) Applicant: BIOGEN MA INC., Cambridge, MA (US)

(72) Inventors: Emily Anne Peterson, Belmont, MA (US); Ryan Evans, Somerville, MA (US); Fang Gao, Somerville, MA (US); Philippe Bolduc, Weymouth, MA (US); Magnus Pfaffenbach, Chelsea, MA (US); Zhili Xin, Lexington, MA (US); Tricia May-Dracka, Third Lake, IL (US)

(21) Appl. No.: 17/623,181

(22) PCT Filed: Jun. 24, 2020
(86) PCT No.: PCT/US2020/039359

§ 371 Date: Dec. 27, 2021

Related U.S. Application Data
(60) Provisional application No. 62/867,589, filed on Jun. 27, 2019.

(51) Int. Cl. C07D 471/04; C07D 487/04; A61K 31/437; A61K 31/4985; A61K 45/06; C07D 519/00; A61K 31/519

(52) U.S. Cl. CPC C07D 471/04; C07D 487/04; A61K 31/437; A61K 31/4985; A61K 45/06; C07D 519/00; A61K 31/519

(57) ABSTRACT

This invention relates to Imidazo[1,2-a]pyridinyl Derivatives of formula (I′), or pharmaceutically acceptable salts thereof, in which all of the variables are as defined in the specification, capable of modulating the activity of IRAK4. The invention further provides a method of manufacturing compounds of the invention, and methods for their therapeutic use. The invention further provides methods to their preparation, to their medical use, in particular to their use in the treatment and management of diseases or disorders including inflammatory disease, autoimmune disease, cancer, cardiovascular disease, a disease of the central nervous system, disease of the skin, an ophthalmic disease and condition, and a bone disease.

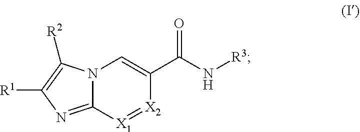

### RELATED APPLICATION

This application claims the benefit of the filing date under 35 U.S.C. § 119(e), of U.S. Provisional Patent Application No. 62/867,589, filed on Jun. 27, 2019, the entire content of which is hereby incorporated by reference.

### FIELD OF THE INVENTION

The present invention relates to Imidazo[1,2-a]pyridinyl Derivatives and pharmaceutically acceptable salts thereof, compositions of these compounds, either alone or in combination with at least one additional therapeutic agent, processes for their preparation, their use in the treatment of diseases, their use, either alone or in combination with at least one additional therapeutic agent and optionally in combination with a pharmaceutically acceptable carrier, for the manufacture of pharmaceutical preparations, use of the pharmaceutical preparations for the treatment of diseases, and a method of treatment of said diseases, comprising administering the Imidazo[1,2-a]pyridinyl Derivatives to a warm-blooded animal, especially a human.

### BACKGROUND OF THE INVENTION

The search for new therapeutic agents has been greatly aided in recent years by a better understanding of the structure of enzymes and other biomolecules associated with diseases. One important class of enzymes that has been the subject of extensive study is the protein kinase family.

Kinases catalyze the phosphorylation of proteins, lipids, sugars, nucleosides and other cellular metabolites and play key roles in all aspects of eukaryotic cell physiology. Especially, protein kinases and lipid kinases participate in the signaling events which control the activation, growth, differentiation and survival of cells in response to extracellular mediators or stimuli such as growth factors, cytokines or chemokines. In general, protein kinases are classified in two groups, those that preferentially phosphorylate tyrosine residues and those that preferentially phosphorylate serine and/or threonine residues.

Kinases are important therapeutic targets for the development of anti-inflammatory drugs (Cohen, 2009. Current Opinion in Cell Biology 21, 1-8), for example kinases that are involved in the orchestration of adaptive and innate immune responses. Kinase targets of particular interest are members of the IRAK family.

The interleukin-1 receptor-associated kinases (IRAKs) are critically involved in the regulation of intracellular signaling networks controlling inflammation (Ringwood and Li, 2008. Cytokine 42, 1-7). IRAKs are expressed in many cell types and can mediate signals from various cell receptors including toll-like receptors (TLRs). IRAK4 is thought to be the initial protein kinase activated downstream of the interleukin-1 (IL-1) receptor and all toll-like-receptors (TLRs) except TLR3, and initiates signaling in the innate immune system via the rapid activation of IRAK1 and slower activation of IRAK2. IRAK1 was first identified through biochemical purification of the IL-1 dependent kinase activity that co-immunoprecipitates with the IL-1 type 1 receptor (Cao et al., 1996. Science 271(5252): 1128-31). IRAK2 was identified by the search of the human expressed sequence tag (EST) database for sequences homologous to IRAK1 (Muzio et al., 1997. Science 278(5343): 1612-5). IRAK3 (also called IRAKM) was identified using a murine EST sequence encoding a polypeptide with significant homology to IRAK1 to screen a human phytohemagglutinin-activated peripheral blood leukocyte (PBL) cDNA library (Wesche et al., 1999. J. Biol. Chem. 274(27): 19403-10). IRAK4 was identified by database searching for IRAK-like sequences and PCR of a universal cDNA library (Li et al., 2002. Proc. Natl. Acad. Sci. USA 99(8):5567-5572). Many diseases are associated with abnormal cellular responses triggered by kinase-mediated events.

Many diseases and/or disorders are associated with abnormal cellular responses triggered by kinase-mediated events. These diseases and/or disorders include, but are not limited to, cancers, allergic diseases, autoimmune diseases, inflammatory diseases and/or disorders and/or conditions associated with inflammation and pain, proliferative diseases, hematopoietic disorders, hematological malignancies, bone disorders, fibrosis diseases and/or disorders, metabolic disorders, muscle diseases and/or disorders, respiratory diseases, pulmonary disorders, genetic development diseases, neurological and neurodegenerative diseases and/or disorders, chronic inflammatory demyelinating neuropathies, cardiovascular, vascular or heart diseases, epilepsy, Ischemic stroke, ophthalmic diseases, ocular diseases, asthma, Alzheimer's disease, Amyotrophic Lateral Sclerosis, Parkinson's disease, traumatic brain injury, Chronic Traumatic Encephalopathy and hormone-related diseases.

In view of the above, IRAK4 inhibitors are considered to be of value in the treatment and/or prevention for multiple therapeutic indications over a wide range of unmet needs.

### SUMMARY OF THE INVENTION

In a first aspect, the invention relates to a compound of formula (I′)

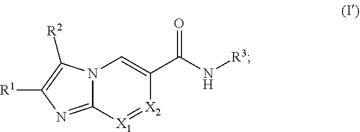

or a pharmaceutically acceptable salt thereof, wherein:

R¹ is selected from the group consisting of halo, C₁₋₅ alkyl, C₃₋₆cycloalkyl, —C₁₋₂ alkyl-C₃₋₆cycloalkyl, a fully saturated 4 to 7 membered heterocycle containing 1 to 2 heteroatoms independently selected from nitrogen, sulfur and oxygen, —C₁₋₂ alkyl-C₄₋₇ heterocycle, wherein the C₄₋₇ heterocycle may be fully or partially saturated and contains 1 to 2 heteroatoms independently selected from nitrogen, sulfur and oxygen, —C₁₋₄ alkyl-O—C₁₋₂ alkyl, a fully saturated 5 to 8 membered bridged-carbocyclic ring, a fully saturated 5 to 8 membered bridged-heterocyclic ring system having 1 to 2 heteroatoms independently selected from nitrogen and oxygen, a 5 to 10 membered fused heterobicyclic ring system having 1 to 2 heteroatoms independently selected from nitrogen and oxygen and a 5 to 10 membered spiro heterobicyclic ring system having 1 to 2 heteroatoms independently selected from nitrogen and oxygen, wherein le may be optionally substituted with 1, 2 or 3 substituents R^{1a} which are independently selected from halo, nitrile, oxo, halo-substituted C₁₋₄ alkyl, hydroxy-substituted C₁₋₄ alkyl, C₁₋₄ alkyl, C₄₋₇ heterocycle containing 1 to 2 heteroatoms independently selected from nitrogen and oxygen, C₁₋₄ alkyl-O—C₁₋₂ alkyl, hydroxyl and C₁₋₄ alkoxy;

R² is hydrogen, C₁₋₄ alkyl or halogen;

R³ is selected from the group consisting of
i. a 5 or 6 membered heteroaryl having 1 to 3 heteroatoms independently selected from nitrogen, oxygen and sulfur, said heteroaryl is optionally substituted with 1 to 3 R⁴;
ii. Phenyl optionally substituted with 1 to 3 R⁴,
iii. a 5-6 membered partially or fully saturated heterocycle having 1 to 2 heteroatoms independently selected from oxygen and nitrogen, said heterocycle may be optionally substituted with 1 to 3 R⁴;
iv. a partially or fully saturated C₃₋₆ cycloalkyl which may be optionally substituted with 1 to 3 R⁴;
v. a 7 to 10 membered fused heterobicyclic ring system having 1, 2 or 3 heteroatoms independently selected from nitrogen and oxygen, said ring system is optionally substituted with 1 to 3 R⁴; and
vi. a 7 to 10 membered fused bicyclic ring system, said ring system is optionally substituted with 1 to 3 R⁴;

X₁ and X₂ are independently selected from N, CH and CR⁵, wherein only one of X₁ or X₂ may be N;

R⁵ is selected from halogen, C₁₋₄alkyl, nitrile and —OR⁶, wherein the C₁₋₄alkyl is optionally substituted with C₁₋₄alkoxy;

R⁶ is hydrogen, C₁₋₅alkyl, C₃₋₆cycloalkyl, a 4 to 7 membered partially or fully saturated heterocycle containing 1 or 2 heteroatoms selected from nitrogen and oxygen, a 5 to 10 membered spiro carbocyclic ring and a 5 to 10 membered spiro heterobicyclic ring system having 1 to 2 heteroatoms independently selected from nitrogen and oxygen, wherein the C₁₋₅alkyl represented by R⁶ is optionally substituted with 1 to 3 substituents R^{6′} independently selected from halogen, hydroxyl, C₁₋₄alkoxy, halo-substituted C₁₋₄alkoxy, C₃₋₆cycloalkyl, phenyl, a 4 to 7 membered partially or fully saturated heterocycle containing 1 or 2 heteroatoms selected from nitrogen and oxygen, an a fully saturated 5 to 8 membered bridged-heterocyclic ring system having 1 to 2 heteroatoms independently selected from nitrogen and oxygen; the C₃₋₆cycloalkyl represented by R⁶ is optionally substituted with 1 to 3 substituents R^{6b} independently selected from halo, C₁₋₄alky, halo-substituted C₁₋₄ alkyl, and C₁₋₄alkoxy; the 4 to 7 membered partially or fully saturated heterocycle, the 5 to 10 membered spiro carbocyclic ring and 5 to 10 membered spiro heterobicyclic ring system represented by R⁶ is optionally substituted with 1 to 3 substituents R^{6c} independently selected from C₁₋₄alky and oxo, and wherein said C₃₋₆cycloalkyl, phenyl, 4 to 7 membered partially or fully saturated heterocycle represented by R^{6a} are optionally substituted with 1 to 3 R⁷;

each R⁷ is independently selected from oxo, halo, halo-substituted C₁₋₄ alkyl and C₁₋₄ alkyl;

R⁴ for each occurrence, is independently selected from CN, hydroxyl, C₁₋₄ alkyl, CN-substituted C₁₋₄ alkyl, oxo, halo, halo-substituted C₁₋₄alkyl, C₁₋₄ alkoxy-C₁₋₄ alkyl, —NR⁸R⁹, C₁₋₄ alkoxy, C₁₋₄ alkoxy-C₁₋₄ alkoxy, hydroxy-substituted C₁₋₄ alkyl, halo-substituted C₁₋₄ alkoxy, C₃₋₆cycloalkyl, —C₁₋₄alkyl-C₃₋₆cycloalkyl, C(O)NR¹⁰R¹¹, a C₄₋₇ heterocycle, and a 5 or 6 membered heteroaryl having 1 to 2 heteroatoms independently selected from nitrogen, oxygen and sulfur, said C₃₋₆cycloalkyl and heteroaryl may be optionally substituted with 1 to 2 substituents independently selected from the group consisting of C₁₋₄ alkyl, hydroxyl and halogen; or two R⁴ groups on the same atom may form a C₃₋₆cycloalkyl, or two R⁴ groups on adjacent ring atoms may form phenyl, C₄₋₆ carbocycle, C₄₋₆ heterocycle, or a 7 membered bridged ring system optionally having 1 heteroatom selected from nitrogen and oxygen, wherein said phenyl, C₃₋₆cycloalkyl C₄₋₆ carbocycle and C₄₋₆ heterocycle may be optionally substituted with 1 to 2 C₁₋₄ alkyl, halo or halo-substituted C₁₋₄alkyl;

R⁸ and R⁹ are each independently selected from hydrogen, —C(O)C₁₋₄ alkyl and C₁₋₄ alkyl; or R⁸ and R⁹ may combine to form a 4 to 6 membered saturated ring optionally containing one additional heteroatom selected from nitrogen or oxygen wherein said additional nitrogen may be optionally substituted with C¹⁻⁴ alkyl; and

R¹⁰ and R¹¹ are each independently selected from hydrogen and C₁₋₄ alkyl.

In one embodiment, the invention relates to a compound of formula (I):

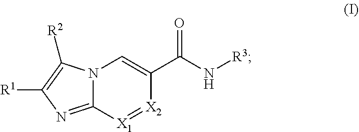

or a pharmaceutically acceptable salt thereof, wherein:

R¹ is selected from the group consisting of C₁₋₅ alkyl, C₃₋₆cycloalkyl, —C₁₋₂ alkyl-C₃₋₆cycloalkyl, a fully saturated 4 to 7 membered heterocycle containing 1 to 2 heteroatoms independently selected from nitrogen, sulfur and oxygen, —C₁₋₂ alkyl-C₄₋₇ heterocycle, wherein the C₄₋₇ heterocycle may be fully or partially saturated and contains 1 to 2 heteroatoms independently selected from nitrogen, sulfur and oxygen, —C₁₋₄ alkyl-O—C₁₋₂ alkyl, a fully saturated 5 to 8 membered bridged-carbocyclic ring, a fully saturated 5 to 8 membered bridged-heterocyclic ring system having 1 to 2 heteroatoms independently selected from nitrogen and oxygen, a 5 to 10 membered fused heterobicyclic ring system having 1 to 2 heteroatoms independently selected from nitrogen and oxygen and a 5 to 10 membered spiro heterobicyclic ring system having 1 to 2 heteroatoms independently selected from nitrogen and oxygen, wherein R¹ may be optionally substituted with 1, 2 or 3 substituents which are independently selected from halo, nitrile, oxo, halo-substituted C₁₋₄ alkyl, hydroxy-substituted C₁₋₄ alkyl, C₁₋₄ alkyl, C₄₋₇ heterocycle containing 1 to 2 heteroatoms independently selected from nitrogen and oxygen, C₁₋₄ alkyl-O—C₁₋₂ alkyl, hydroxyl and C₁₋₄ alkoxy;

R² is hydrogen, C₁₋₄ alkyl or halogen;

R³ is selected from the group consisting of
i. a 5 or 6 membered heteroaryl having 1 to 2 heteroatoms independently selected from nitrogen, oxygen and sulfur, said heteroaryl is optionally substituted with 1 to 3 R⁴;
ii. Phenyl optionally substituted with 1 to 3 R⁴,
iii. a 5-6 membered partially or fully saturated heterocycle having 1 to 2 heteroatoms independently selected from oxygen and nitrogen, said heterocycle may be optionally substituted with 1 to 3 R⁴;
iv. a partially or fully saturated C₃₋₆ cycloalkyl which may be optionally substituted with 1 to 3 R⁴;
v. a 7 to 10 membered fused heterobicyclic ring system having 1, 2 or 3 heteroatoms independently selected from nitrogen and oxygen, said ring system is optionally substituted with 1 to 3 R⁴; and
vi. a 7 to 10 membered fused bicyclic ring system, said ring system is optionally substituted with 1 to 3 R⁴;

X₁ and X₂ are independently selected from N, CH and CR⁵, wherein only one of X₁ or X₂ may be N;

R⁵ is selected from halogen, C₁₋₄alkyl, nitrile and —OR⁶;

R⁶ is hydrogen or an optionally substituted C₁₋₅alkyl having 1 to 3 substituents independently selected from halogen, hydroxyl, C₁₋₄alkoxy, C₃₋₆cycloalkyl, phenyl and a 4 to 7 membered partially or fully saturated heterocycle containing 1 or 2 heteroatoms selected from nitrogen and oxygen, wherein said C₃₋₆cycloalkyl and phenyl may be optionally substituted with 1 to 3 R⁷;

each R⁷ is independently selected from oxo, halo, halo-substituted C₁₋₄ alkyl and C₁₋₄ alkyl;

R⁴ for each occurrence, is independently selected from CN, hydroxyl, C₁₋₄ alkyl, CN-substituted C₁₋₄ alkyl, oxo, halo, halo-substituted C₁₋₄alkyl, —NR⁸R⁹, C₁₋₄ alkoxy, C₁₋₄ alkoxy-C₁₋₄ alkoxy, hydroxy-substituted C₁₋₄ alkyl, halo-substituted C₁₋₄ alkoxy, C₃₋₆cycloalkyl, C(O)NR¹⁰R¹¹ and a 5 or 6 membered heteroaryl having 1 to 2 heteroatoms independently selected from nitrogen, oxygen and sulfur, said C₃₋₆cycloalkyl and heteroaryl may be optionally substituted with 1 to 2 substituents independently selected from the group consisting of C₁₋₄ alkyl, hydroxyl and halogen; or two R⁴ groups on the same atom may form a C₃₋₆cycloalkyl, or two R⁴ groups on adjacent ring atoms may form phenyl, C₄₋₆ carbocycle, C₄₋₆ heterocycle, or a 7 membered bridged ring system optionally having 1 heteroatom selected from nitrogen and oxygen, wherein said phenyl, C₃₋₆cycloalkyl C₄₋₆ carbocycle and C₄₋₆ heterocycle may be optionally substituted with 1 to 2 C₁₋₄ alkyl, halo or halo-substituted C₁₋₄alkyl;

R⁸ and R⁹ are each independently selected from hydrogen, —C(O)C₁₋₄ alkyl and C₁₋₄ alkyl; or R⁸ and R⁹ may combine to form a 4 to 6 membered saturated ring optionally containing one additional heteroatom selected from nitrogen or oxygen wherein said additional nitrogen may be optionally substituted with C₁₋₄ alkyl; and

R¹⁰ and R¹¹ are each independently selected from hydrogen and C₁₋₄ alkyl; or a pharmaceutically acceptable salt thereof.

Another aspect of the invention relates to pharmaceutical compositions comprising compounds of formula (I′) or (I) or pharmaceutically acceptable salts thereof, and a pharmaceutical carrier. Such compositions can be administered in accordance with a method of the invention, typically as part of a therapeutic regimen for the treatment or prevention of conditions and disorders related to interleukin-1 receptor-associated kinases activity. In a particular aspect, the pharmaceutical compositions may additionally comprise further one or more therapeutically active ingredients suitable for the use in combination with the compounds of the invention. In a more particular aspect, the further therapeutically active ingredient is an agent for the treatment of autoimmune diseases, inflammatory diseases, bone diseases, metabolic diseases, neurological and neurodegenerative diseases, cancer, cardiovascular diseases, allergies, asthma, Alzheimer's disease, and hormone-related diseases.

Another aspect of the invention relates to the pharmaceutical combinations comprising compounds of the invention and other therapeutic agents for the use as a medicament in the treatment of patients having disorders related to interleukin-1 receptor-associated kinases activity. Such combinations can be administered in accordance with a method of the invention, typically as part of a therapeutic regiment for the treatment or prevention of autoimmune diseases, inflammatory diseases, bone diseases, metabolic diseases, neurological and neurodegenerative diseases, cancer, cardiovascular diseases, allergies, asthma, Alzheimer's disease, and hormone-related diseases. Accordingly, there remains a need to find protein kinase inhibitors useful as therapeutic agents.

### DETAILED DESCRIPTION OF THE INVENTION

The present invention provides compounds and pharmaceutical formulations thereof that may be useful in the treatment or prevention of conditions and/or disorders through mediation of IRAK4 function, such as neurological and neurodegenerative diseases, Alzheimer's disease, Ischemic stroke, Cerebral Ischemia, hypoxia, TBI (Traumatic Brain Injury), CTE (Chronic Traumatic Encephalopathy), epilepsy, Parkinson's disease (PD), Multiple Sclerosis (MS) and Amyotrophic Lateral Sclerosis (ALS).

In a first embodiment, the invention provides a compound of formula (I′):

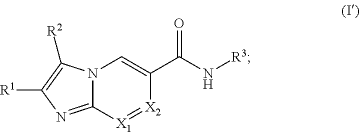

or a pharmaceutically acceptable salt thereof, wherein the variables in formula (I′) are as defined in the first aspect above.

In a second embodiment, the invention provides a compound of formula (I):

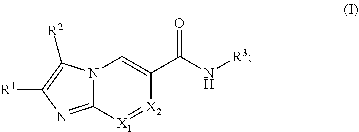

or a pharmaceutically acceptable salt thereof, wherein:

R¹ is selected from the group consisting of C₁₋₅ alkyl, C₃₋₆cycloalkyl, —C₁₋₂ alkyl-C₃₋₆ cycloalkyl, a fully saturated 4 to 7 membered heterocycle containing 1 to 2 heteroatoms independently selected from nitrogen, sulfur and oxygen, —C₁₋₂ alkyl-C₄₋₇ heterocycle, wherein the C₄₋₇ heterocycle may be fully or partially saturated and contains 1 to 2 heteroatoms independently selected from nitrogen, sulfur and oxygen, —C₁₋₄ alkyl-O—C₁₋₂ alkyl, a fully saturated 5 to 8 membered bridged-carbocyclic ring, a fully saturated 5 to 8 membered bridged-heterocyclic ring system having 1 to 2 heteroatoms independently selected from nitrogen and oxygen, a 5 to 10 membered fused heterobicyclic ring system having 1 to 2 heteroatoms independently selected from nitrogen and oxygen and a 5 to 10 membered spiro heterobicyclic ring system having 1 to 2 heteroatoms independently selected from nitrogen and oxygen, wherein R¹ may be optionally substituted with 1, 2 or 3 substituents which are independently selected from halo, nitrile, oxo, halo-substituted C₁₋₄ alkyl, hydroxy-substituted C₁₋₄ alkyl, C₁₋₄ alkyl, C₄₋₇ heterocycle containing 1 to 2 heteroatoms independently selected from nitrogen and oxygen, C₁₋₄ alkyl-O—C₁₋₂ alkyl, hydroxyl and C₁₋₄ alkoxy;

R² is hydrogen, C₁₋₄ alkyl or halogen;

R³ is selected from the group consisting of
i. a 5 or 6 membered heteroaryl having 1 to 2 heteroatoms independently selected from nitrogen, oxygen and sulfur, said heteroaryl is optionally substituted with 1 to 3 R⁴;
ii. Phenyl optionally substituted with 1 to 3 R⁴,
iii. a 5-6 membered partially or fully saturated heterocycle having 1 to 2 heteroatoms independently selected from oxygen and nitrogen, said heterocycle may be optionally substituted with 1 to 3 R⁴;
iv. a partially or fully saturated C₃₋₆ cycloalkyl which may be optionally substituted with 1 to 3 R⁴;
v. a 7 to 10 membered fused heterobicyclic ring system having 1, 2 or 3 heteroatoms independently selected from nitrogen and oxygen, said ring system is optionally substituted with 1 to 3 R⁴; and
vi. a 7 to 10 membered fused bicyclic ring system, said ring system is optionally substituted with 1 to 3 R⁴;

X₁ and X₂ are independently selected from N, CH and CR⁵, wherein only one of X₁ or X₂ may be N;

R⁵ is selected from halogen, C₁₋₄alkyl, nitrile and —OR⁶;

R⁶ is hydrogen or an optionally substituted C₁₋₅alkyl having 1 to 3 substituents independently selected from halogen, hydroxyl, C₁₋₄alkoxy, C₃₋₆cycloalkyl, phenyl and a 4 to 7 membered partially or fully saturated heterocycle containing 1 or 2 heteroatoms selected from nitrogen and oxygen, wherein said C₃₋₆cycloalkyl and phenyl may be optionally substituted with 1 to 3 R⁷;

each R⁷ is independently selected from oxo, halo, halo-substituted C₁₋₄ alkyl and C₁₋₄ alkyl;

R⁴ for each occurrence, is independently selected from CN, hydroxyl, C₁₋₄ alkyl, CN-substituted C₁₋₄ alkyl, oxo, halo, halo-substituted C₁₋₄alkyl, —NR⁸R⁹, C₁₋₄ alkoxy, C₁₋₄ alkoxy-C₁₋₄ alkoxy, hydroxy-substituted C₁₋₄ alkyl, halo-substituted C₁₋₄ alkoxy, C₃₋₆cycloalkyl, C(O)NR¹⁰R¹¹ and a 5 or 6 membered heteroaryl having 1 to 2 heteroatoms independently selected from nitrogen, oxygen and sulfur, said C₃₋₆cycloalkyl and heteroaryl may be optionally substituted with 1 to 2 substituents independently selected from the group consisting of C₁₋₄ alkyl, hydroxyl and halogen; or two R⁴ groups on the same atom may form a C₃₋₆cycloalkyl, or two R⁴ groups on adjacent ring atoms may form phenyl, C₄₋₆ carbocycle, C₄₋₆ heterocycle, or a 7 membered bridged ring system optionally having 1 heteroatom selected from nitrogen and oxygen, wherein said phenyl, C₃₋₆cycloalkyl C₄₋₆ carbocycle and C₄₋₆ heterocycle may be optionally substituted with 1 to 2 C₁₋₄ alkyl, halo or halo-substituted C₁₋₄alkyl;

R⁸ and R⁹ are each independently selected from hydrogen, —C(O)C₁₋₄ alkyl and C₁₋₄ alkyl; or R⁸ and R⁹ may combine to form a 4 to 6 membered saturated ring optionally containing one additional heteroatom selected from nitrogen or oxygen wherein said additional nitrogen may be optionally substituted with C₁₋₄ alkyl; and

R¹⁰ and R₁₁ are each independently selected from hydrogen and C₁₋₄ alkyl.

In a third embodiment, the invention provides a compound of the first or second embodiment of formula (I):

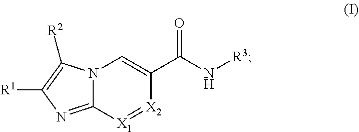

or a pharmaceutically acceptable salt thereof, wherein:

R² is H; and

X₁ is N or CH; and X² is CR⁵; and the remaining variables are as defined in the first or second embodiment.

In a fourth embodiment, the invention provides a compound of the first or second embodiment of formula (I):

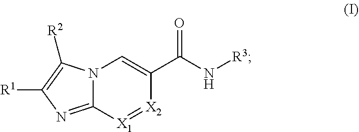

or a pharmaceutically acceptable salt thereof, wherein:

### R² is H; and

X₁ is CR⁵ and X² is N or CH; and the remaining variables are as defined in the first or second embodiment.

In a fifth embodiment, the invention provides a compound of the first or second embodiment of formula (Ia):

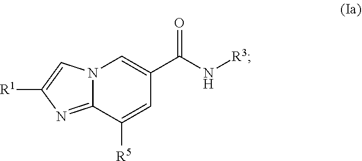

or a pharmaceutically acceptable salt thereof, wherein the variables are as defined in the first or second embodiment.

In a sixth embodiment, the invention provides a compound of the first or second embodiment of formula (Ib):

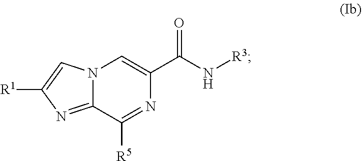

or a pharmaceutically acceptable salt thereof, wherein the variables are as defined in the first or second embodiment.

In a seventh embodiment, the invention provides a compound of the first or second embodiment of formula (Ic):

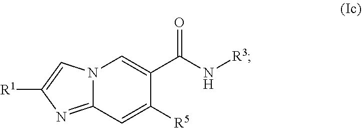

or a pharmaceutically acceptable salt thereof, wherein the variables are as defined in the first or second embodiment.

In an eighth embodiment, the invention provides a compound of the first or second embodiment of formula (Id):

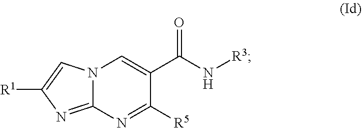

or a pharmaceutically acceptable salt thereof, wherein the variables are as defined in the first or second embodiment.

A ninth embodiment of the invention provides a compound according to any of the preceding embodiments or a pharmaceutically acceptable salt thereof, wherein:

R³ is selected from the group consisting of
i. a 5 or 6 membered heteroaryl having 1 to 2 heteroatoms independently selected from nitrogen, oxygen and sulfur, said heteroaryl is optionally substituted with 1 to 3 R⁴;
ii. Phenyl optionally substituted with 1 to 3 R⁴,
iii. a 5-6 membered partially or fully saturated heterocycle having 1 to 2 heteroatoms independently selected from oxygen and nitrogen, said heterocycle may be optionally substituted with 1 to 3 R⁴;
iv. a partially or fully saturated C₃₋₆ cycloalkyl which may be optionally substituted with 1 to 3 R⁴;
v. a 7 to 10 membered fused heterobicyclic ring system having 1, 2 or 3 heteroatoms independently selected from nitrogen and oxygen, said ring system is optionally substituted with 1 to 3 R⁴; and
vi. a 7 to 10 membered fused bicyclic ring system, said ring system is optionally substituted with 1 to 3 R⁴; and
   the remaining variables are as defined in the first, second, third, fourth, fifth, sixth, seventh or eighth embodiment.

In a tenth embodiment, the invention provides a compound of any one of the preceding embodiments or a pharmaceutically acceptable salt thereof, wherein:

R³ is a 5 or 6 membered monocyclic heteroaryl having 1 to 2 heteroatoms independently selected from nitrogen and oxygen, pyridinyl-2(1H)-one or a 9 to 10 membered bicyclic heteroaryl having 1 to 3 heteroatoms independently selected from nitrogen and oxygen, wherein the monocyclic heteroaryl, pyridinyl-2(1H)-one or the bicyclic heteroaryl are each optionally substituted with 1 or 2 R⁴; and the remaining variables are as defined in the ninth embodiment.

In an eleventh embodiment, the invention provides a compound of any one of the preceding embodiments or a pharmaceutically acceptable salt thereof, wherein:

R³ is a 5 or 6 membered monocyclic heteroaryl having 1 to 2 nitrogen atoms, pyridinyl-2(1H)-one or a 9 to 10 membered bicyclic heteroaryl having 2 to 3 nitrogen atoms, wherein the monocyclic heteroaryl, pyridinyl-2(1H)-one or the bicyclic heteroaryl are each optionally substituted with 1 or 2 R⁴; and the remaining variables are as defined in the tenth embodiment.

In a twelfth embodiment, the invention provides a compound of any one of the first to eleventh embodiments or a pharmaceutically acceptable salt thereof, wherein:

R⁴, for each occurrence, is independently selected from hydroxyl, halo, halo-substituted C₁₋₄ alkyl, —NR⁸R⁹, C₁₋₄alkoxy, C₃₋₆cycloalkyl, and C₁₋₄ alkyl; and the remaining variables are as defined in the ninth, tenth or eleventh embodiment. In one embodiment, R⁴, for each occurrence, is independently selected from hydroxyl, halo, halo-substituted C₁₋₄ alkyl, —NR⁸R⁹, and C₁₋₄ alkyl; and the remaining variables are as defined in any one of first to eleventh embodiment.

In a thirteenth embodiment, the invention provides a compound of any one of the first to eighth embodiments or a pharmaceutically acceptable salt thereof, wherein:

R³ is selected from pyridyl, oxazolyl, pyrazinyl, oxadiazoyl, thiophenyl, thiazolyl, isothiazolyl, pyrazolyl, imidazolyl, said R³ is optionally substituted with 1 to 2 substituents independently selected from the group consisting of halo, halo-substituted C₁₋₄ alkyl, —NR⁸R⁹, and C₁₋₄ alkyl; and the remaining variables are as defined in the first, second, third, fourth, fifth, sixth, seventh or eighth embodiment.

In a fourteenth embodiment, the invention provides a compound of any one of the first to eighth embodiments or a pharmaceutically acceptable salt thereof, wherein:

R³ is pyridinyl-2(1H)-one optionally substituted with 1 to 2 substituents independently selected from the group consisting of halo, halo-substituted C₁₋₄ alkyl, —NR⁸R⁹, and C₁₋₄ alkyl; and the remaining variables are as defined in the first, second, third, fourth, fifth, sixth, seventh or eighth embodiment.

In a fifteenth embodiment, the invention provides a compound of any one of the first to eighth embodiments or a pharmaceutically acceptable salt thereof, wherein:

R³ is phenyl, said phenyl is optionally substituted with 1 to 2 substituents independently selected from the group consisting of halo, halo-substituted C₁₋₄ alkyl, —NR⁸R⁹, and C₁₋₄ alkyl; and the remaining variables are as defined in the first, second, third, fourth, fifth, sixth, seventh or eighth embodiment.

In a sixteenth embodiment, the invention provides a compound of any one of the first to eighth embodiments or a pharmaceutically acceptable salt thereof, wherein:

R³ is selected from the group consisting of 1,3-dihydroisobenzofuran, 2,3-dihydrobenzofuran, 4-oxaspiro[bicyclo[3.2.0]heptane-6,1′-cyclobutane], oxaspiro[bicyclo[3.2.0]heptane-6,1′-cyclobutane], bicyclo[3.1.0]hexane, cyclohexyl, spiro[2.5]octane, (1 S,5R)-1-methylbicyclo[3.1.0]hexane, spiro[2.5]octane, 1,2,3,4-tetrahydronaphthalen, tetrahydrofuran, 2,3-dihydrobenzofuran, 2,3-dihydro-1H-indene, 4-methyl-3,4-dihydro-2H-benzo[b][1,4]oxazine, pyrido[3,2-d]pyrimidinyl, 1,2,3,4-tetrahydro-1,4-epoxynaphthalene, 5,6-dihydro-4H-pyrrolo[1,2-b]pyrazole, 6,7-dihydro-5H-cyclopenta[b]pyridine, 1,2,3,4-tetrahydronaphthalene, indolin-2-one, 2,3-dihydrobenzofuran, pyrazolo[1,5-a]pyrimidine, 1-methyl-2-oxo-1,2,3,4-tetrahydroquinoline, 3,4-dihydroquinolin-2(1H)-one, chromane, and isochromane, wherein said R³ is optionally substituted with 1 to 2 substituents independently selected from the group consisting of halo, halo-substituted C₁₋₄ alkyl, —NR⁸R⁹, and C₁₋₄ alkyl; and the remaining variables are as defined in the first, second, third, fourth, fifth, sixth, seventh or eighth embodiment.

In a seventeenth embodiment, the invention provides a compound of any one of the first to eighth embodiments or a pharmaceutically acceptable salt thereof, wherein:

R³ is selected from the group consisting of cyclopropyl, cyclobutyl, cyclohexyl, bicyclo[3.1.0]hexane, bicyclo[4.1.0]heptane, tetrahydrofuran, 4-oxaspiro[bicyclo[3.2.0]heptane-6,1′-cyclobutane], oxaspirobicyclo[3.2.0]heptane, spiro[2.5]octane, phenyl, 2H-1,2,3-triazole, isoxazole, isothiazole, thiazole, pyrazole, pyridine, pyridinyl-2(1H)-one, 6,7-dihydro-5H-cyclopenta[b]pyridine, pyrazolo[1,5-a]pyridine, [1,2,4]triazolo[4,3-a]pyridine, isothiazolo[4,3-b]pyridine, pyrimidine, pyrimidin-4(3H)-one, pyrazolo[1,5-a]pyrimidine, pyrido[3,2-d]pyrimidine, imidazo[1,2-b]pyridazine, thieno[2,3-b]pyrazine, 1H-benzo[d]imidazole, benzo[d]thiazole, 2,3-dihydrobenzofuran, indane, 2,3-dihydro-1H-indene, 1,6-naphthyridine, 1,5-naphthyridine, 5,6,7,8-tetrahydronaphthalene, 2H-indazole, 6,7-dihydro-5H-pyrazolo[5,1-b][1,3]oxazine, thiophene, chromane and isochromane, and the remaining variables are as defined in the first, second, third, fourth, fifth, sixth, seventh or eighth embodiment. In one embodiment, for compounds described in the seventeenth embodiment or a pharmaceutically acceptable salt thereof, the R³ group is optionally substituted with 1 to 3 (e.g. 1 or 2) R⁴ independently selected from hydroxyl, halo, halo-substituted C₁₋₄ alkyl, —NR⁸R⁹, C₁₋₄alkoxy, C₃₋₆cycloalkyl, and C₁₋₄ alkyl. In another embodiment, for compounds described in the seventh embodiment or a pharmaceutically acceptable salt thereof, the R³ group is optionally substituted with 1 to 2 R⁴ independently selected from hydroxyl, halo, halo-substituted C₁₋₄ alkyl, —NR⁸R⁹, and C₁₋₄ alkyl

In an eighteenth embodiment, the invention provides a compound of any one of the first to eighth embodiments or a pharmaceutically acceptable salt thereof, wherein:

R³ is selected from the group consisting of: 2-cyclobutylcyclopropyl, (1R,2S)-2-cyclobutylcyclopropyl, 3-methylcyclobutyl, 2,3-dimethylcyclohexyl, 3-fluorocyclohexyl, 2-methoxycyclohexyl, (1R,2R)-2-methoxycyclohexyl, 3-cyclopropylcyclohexyl, (1R,3S)-3-cyclopropylcyclohexyl, (1S,4S)-4-methoxycyclohexyl, 4-methoxycyclohexyl, bicyclo[3.1.0]hexan-1-yl, (1R,5R)-bicyclo[3.1.0]hexan-1-yl, 7,7-difluorobicyclo[4.1.0]heptan-2-yl, 4-fluorotetrahydrofuran-3-yl, 4-oxaspiro[bicyclo[3.2.0]heptane-6,1′-cyclobutan]-7-yl, spiro[2.5]octan-5-yl, 3-chlorophenyl, 3,5-dichlorophenyl, 2-fluorophenyl, 3-fluorophenyl, 2,3-difluorophenyl, 3,5-difluorophenyl, 2,3,5-trifluorophenyl, 3,4,5-trifluorophenyl, 3-chloro-2-fluorophenyl, 2-chloro-3-fluorophenyl, 3-chloro-5-fluorophenyl, 3,5-dichloro-4-fluorophenyl, 3-cyano-2-fluorophenyl, m-tolyl, 2,3-dimethylphenyl, 3,5-dimethylphenyl, 2-ethylphenyl, 2-isobutylphenyl, 3-cyclopropylphenyl, 3-(fluoromethyl)phenyl, 3-(difluoromethyl)phenyl, 3-(trifluoromethyl)phenyl, 3-(difluoromethyl)-4-fluorophenyl, 3-(difluoromethyl)-5-fluorophenyl, 3-(difluoromethyl)-4,5-difluorophenyl, 2-methyl-3-(trifluoromethyl)phenyl, 2-fluoro-3-(trifluoromethyl)phenyl, 2-fluoro-3-methylphenyl, 3-fluoro-2-methylphenyl, 3-fluoro-5-methylphenyl, 3,4-difluoro-2-methylphenyl, 3-(1,1-difluoroethyl)phenyl, 3-(1,1,2-trifluoroethyl)phenyl, 2-chloro-3-methylphenyl, 3-chloro-2-methylphenyl, 3-methoxyphenyl, 3-methoxy-2-methylphenyl, 2-methoxy-3,5-dimethylphenyl, 3-chloro-2-methoxyphenyl, 5-chloro-2-methoxyphenyl, 4-fluoro-2-methoxyphenyl, 3-fluoro-2-methoxyphenyl, 3-fluoro-5-methoxyphenyl, 5-fluoro-2-methoxyphenyl, 2-isopropoxyphenyl, 5-fluoro-2-isopropoxyphenyl, 4-fluoro-2-isopropoxyphenyl, 2-methyl-2H-1,2,3-triazol-4-yl, 3-methylisoxazol-4-yl, isothiazol-4-yl, isoxazol-5-yl, thiazol-2-yl, 4-methylthiazol-5-yl, 4-ethylthiazol-5-yl, 4-isopropylthiazol-5-yl, 4-(difluoromethyl)thiazol-2-yl, 5-chloro-4-methylthiazol-2-yl, 4-(trifluoromethyl)thiazol-2-yl, 3-methoxyisothiazol-4-yl, 1-methyl-1H-pyrazol-3-yl, 1, 5-dimethyl-1H-pyrazol-4-yl, 1-ethyl-1H-pyrazol-3-yl, 5-ethyl-1-methyl-1H-pyrazol-4-yl, 5-fluoro-1-methyl-1H-pyrazol-3-yl, 1-(difluoromethyl)-1H-pyrazol-3-yl, 1-(trifluoromethyl)-1H-pyrazol-3-yl, 1-(2,2-difluoroethyl)-1H-pyrazol-3-yl, 1-(2-fluoroethyl)-1H-pyrazol-3-yl, 1-cyclopropyl-1H-pyrazol-3-yl, 1-(cyclopropylmethyl)-1H-pyrazol-3-yl, 5-cyclopropyl-1-methyl-1H-pyrazol-4-yl, 1-(2,2-difluorocyclopropyl)-1H-pyrazol-3-yl, 1-cyclobutyl-1H-pyrazol-3-yl, 1-cyclopentyl-1H-pyrazol-3-yl, 1-(cyanomethyl)-1H-pyrazol-3-yl, 1-(2-methoxyethyl)-1H-pyrazol-3-yl, 1-(2-methylpyridin-4-yl)-1H-pyrazol-3-yl, pyridin-2-yl, 6-cyanopyridin-2-yl, 4-fluoropyridin-2-yl, 5-fluoropyridin-2-yl, 6-(cyanomethyl)pyridin-2-yl, 2-methylpyridin-3-yl, 6-methylpyridin-2-yl, 4,6-dimethylpyridin-2-yl, 6-(difluoromethyl)pyridin-2-yl, 2-(difluoromethyl)pyridin-4-yl, 6-ethylpyridin-2-yl, (2-ethyl-5-fluoropyridin-3-yl, 6-(1,2-difluoroethyl)pyridin-2-yl, 6-(trifluoromethyl)pyridin-2-yl, 6-(1,1-difluoroethyl)pyridin-2-yl, 2-isopropylpyridin-3-yl, 2-cyclopropylpyridin-3-yl, 6-cyclopropylpyridin-2-yl, 2-(difluoromethoxy)pyridin-3-yl, 6-(difluoromethoxy)pyridin-2-yl, 6-(trifluoromethoxy)pyridin-2-yl, 2-methoxypyridin-3-yl, 3-methoxypyridin-4-yl, 6-methoxypyridin-2-yl, 2-(2,2-difluoroethoxy)pyridin-3-yl, 6-(2,2-difluoroethoxy)pyridin-2-yl, 6-ethoxypyridin-2-yl, 2-isopropoxypyridin-3-yl, 2-hydroxypyridin-3-yl, 6-(hydroxymethyl)pyridin-2-yl, 6-hydroxy-2-methoxypyridin-3-yl, 3-methoxy-2-methylpyridin-4-yl, 5-fluoro-2-methoxypyridin-3-yl, 6-(dimethylamino)pyridin-2-yl, 1-methyl-2-oxo-1,2-dihydropyridin-3-yl, 1,6-dimethyl-2-oxo-1,2-dihydropyridin-3-yl, 1-ethyl-2-oxo-1,2-dihydropyridin-3-yl, 1-(difluoromethyl)-2-oxo-1,2-dihydropyridin-3-yl, 5-fluoro-1-methyl-2-oxo-1,2-dihydropyridin-3-yl, 5-cyano-1-methyl-2-oxo-1,2-dihydropyridin-3-yl, 1-isopropyl-2-oxo-1,2-dihydropyridin-3-yl, 2-oxo-1-(2,2,2-trifluoroethyl)-1,2-dihydropyridin-3-yl, 6-(tetrahydrofuran-3-yl)pyridin-2-yl, 6,7-dihydro-5H-cyclopenta[b]pyridin-2-yl, 6-(isoxazol-4-yl)pyridin-2-yl, 6-(oxazol-5-yl)pyridin-2-yl, pyrazolo[1,5-a]pyridin-2-yl, pyrazolo[1,5-a]pyridin-4-yl, pyrazolo[1,5-a]pyridin-7-yl, 6-(1-methyl-1H-pyrazol-4-yl)pyridin-2-yl, 4-fluoropyrazolo[1,5-a]pyridin-3-yl, 4-methoxypyrazolo[1,5-a]pyridin-3-yl, [1,2,4]triazolo[4,3-a]pyridin-8-yl, [1,2,4]triazolo[1,5-a]pyridin-5-yl, isothiazolo[4,3-b]pyridin-3-yl, 4-(difluoromethyl)pyrimidin-2-yl, 1-methyl-6-oxo-1,6-dihydropyrimidin-5-yl, pyrazolo[1,5-a]pyrimidin-3-yl, pyrazolo[1,5-a]pyrimidin-7-yl, pyrazolo[1,5-a]pyrimidin-5-yl, 5-methylpyrazolo[1,5-a]pyrimidin-3-yl, 6-methylpyrazolo[1,5-a]pyrimidin-3-yl, 6-fluoropyrazolo[1,5-a]pyrimidin-3-yl, 5-(difluoromethyl)pyrazolo[1,5-a]pyrimidin-3-yl, 6-(difluoromethyl)pyrazolo[1,5-a]pyrimidin-3-yl, 5-chloropyrazolo[1,5-a]pyrimidin-3-yl, 5-methoxypyrazolo[1,5-a]pyrimidin-3-yl, 6-methoxypyrazolo[1,5-a]pyrimidin-3-yl, 6-cyclopropylpyrazolo[1,5-a]pyrimidin-3-yl, 3-chloropyrrolo[1,2-a]pyrimidin-8-yl, pyrido[3,2-d]pyrimidin-4-yl, imidazo[1,2-b]pyridazin-3-yl, 6-methoxyimidazo[1,2-b]pyridazin-3-yl, thieno[2,3-b]pyrazin-7-yl, 1-methyl-1H-benzo[d]imidazol-4-yl, benzo[d]thiazol-4-yl, 2,3-dihydrobenzofuran-7-yl, 2,3-dihydrobenzofuran-4-yl, indan-4-yl, 2,3-dihydro-1H-inden-4-yl, 3-methoxy-2,3-dihydro-1H-inden-1-yl, 1, 6-naphthyridin-8-yl, 1, 5-naphthyridin-4-yl, 5,6,7,8-tetrahydronaphthalen-1-yl, 1,2,3,4-tetrahydro-1,4-epoxynaphthalen-5-yl, 2-methyl-2H-indazol-7-yl, 6,7-dihydro-5H-pyrazolo[5,1-b)][1,3]oxazin-3-yl, 4-chlorothiophen-3-yl, 4-methylthiophen-3-yl, chroman-8-yl, and isochroman-5-yl.

In a nineteenth embodiment, the invention provides a compound of any one of embodiments one, two, three and four of formula (II):

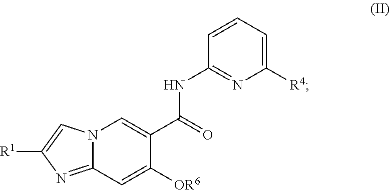

or a pharmaceutically acceptable salt thereof, wherein:

R⁶ is an optionally substituted C₁₋₅alkyl having 1 to 3 substituents independently selected from halogen, hydroxyl, C₁₋₄alkoxy, C₃₋₆cycloalkyl, phenyl and a 4 to 7 membered partially or fully saturated heterocycle containing 1 or 2 heteroatoms selected from nitrogen and oxygen, wherein said C₃₋₆cycloalkyl and phenyl may be optionally substituted with 1 to 3 R⁷; and the remaining variables are as defined in the first, second, third or fourth embodiment.

In an twentieth embodiment, the invention provides a compound of any of one of embodiments one, two, three and four of formula (III):

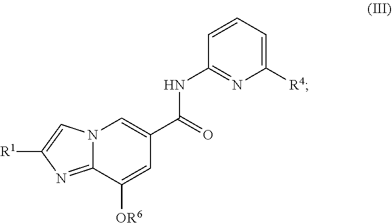

or a pharmaceutically acceptable salt thereof, wherein:

R⁶ is an optionally substituted C₁₋₅alkyl having 1 to 3 substituents independently selected from halogen, hydroxyl, C₁₋₄alkoxy, C₃₋₆cycloalkyl, phenyl and a 4 to 7 membered partially or fully saturated heterocycle containing 1 or 2 heteroatoms selected from nitrogen and oxygen, wherein said C₃₋₆cycloalkyl and phenyl may be optionally substituted with 1 to 3 R⁷; the remaining variables are as defined in the first, second, third or fourth embodiment.

In a twenty-first embodiment, the invention provides a compound of any of one of embodiments one, two or three of formula (IV):

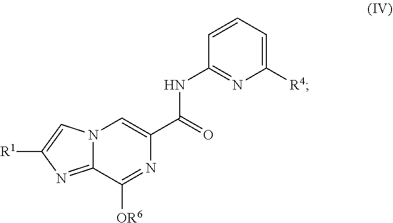

or a pharmaceutically acceptable salt thereof, wherein:

R⁶ is an optionally substituted C₁₋₅alkyl having 1 to 3 substituents independently selected from halogen, hydroxyl, C₁₋₄alkoxy, C₃₋₆cycloalkyl, phenyl and a 4 to 7 membered partially or fully saturated heterocycle containing 1 or 2 heteroatoms selected from nitrogen and oxygen, wherein said C₃₋₆cycloalkyl and phenyl may be optionally substituted with 1 to 3 R⁷; the remaining variables are as defined in the first, second, third or fourth embodiment.

In a twenty-second embodiment, the invention provides a compound of any one of the preceding embodiments or a pharmaceutically acceptable salt thereof, wherein:

R¹ is a fully saturated C₄₋₇ heterocycle or a 5 to 8 membered bridged-heterocyclic ring system which contain 1 to 2 heteroatoms independently selected from nitrogen and oxygen, said C₄₋₇ heterocycle or a 5 to 8 membered bridged-heterocyclic ring system may be optionally substituted with 1 or 2 substituents independently selected from the group consisting of C₁₋₄alkyl, halogen, halo-substituted C₁₋₄ alkyl, hydroxyl and C₁₋₄alkoxy; or R^{1—} is a C₁₋₅ alkyl which is optionally substituted with 1 or 3 substituents independently selected from the group consisting of halogen, halo-substituted C₁₋₄ alkyl, hydroxy-substituted C₁₋₄ alkyl, hydroxyl, C₁₋₄alkoxy and C₃₋₆cycloalkyl, wherein said C₃₋₆cycloalkyl is optionally substituted with 1 or 2 substituents independently selected from the group consisting of halogen, halo-substituted C₁₋₄ alkyl, hydroxyl and C₁₋₄alkoxy; and the remaining variables are as defined in any one of the first to twenty-first embodiments.

In a twenty-third embodiment of the invention provides a compound of any one of the preceding embodiments or a pharmaceutically acceptable salt thereof, wherein:

R¹ is a fully saturated C₄₋₇ heterocycle or a 5 to 8 membered bridged-heterocyclic ring system which contain 1 to 2 heteroatoms independently selected from nitrogen and oxygen, said C₄₋₇ heterocycle or a 5 to 8 membered bridged-heterocyclic ring system may be optionally substituted with 1 or 2 substituents independently selected from the group consisting of C₁₋₄alkyl, halogen, halo-substituted C₁₋₄ alkyl, hydroxyl and C₁₋₄alkoxy; and the remaining variables are as defined in any one of the first to twenty-first embodiments.

In a twenty-fourth embodiment, the invention provides a compound of any one of the first to twenty-first embodiments or a pharmaceutically acceptable salt thereof, wherein R¹ is a C₁₋₅ alkyl which is optionally substituted with 1 or 3 substituents independently selected from the group consisting of halogen, halo-substituted C₁₋₄ alkyl, hydroxyl, C₁₋₄alkoxy and C₃₋₆cycloalkyl, wherein said C₃₋₆cycloalkyl is optionally substituted with 1 or 2 substituents independently selected from the group consisting of halogen, halo-substituted C₁₋₄ alkyl, hydroxyl and C₁₋₄alkoxy; and the remaining variables are as defined in any one of the first to twenty-first embodiments.

In a twenty-fifth embodiment, the invention provides a compound of any one of the first to twenty-first embodiments or a pharmaceutically acceptable salt thereof, wherein R¹ is a C₁₋₅ alkyl substituted with 1 or 3 substituents independently selected from the group consisting of halo-substituted C₁₋₄ alkyl, hydroxyl, C₁₋₄alkoxy and C₃₋₆cycloalkyl, wherein said C₃₋₆cycloalkyl is optionally substituted with 1 or 2 substituents independently selected from the group consisting of halogen, halo-substituted C₁₋₄ alkyl, hydroxyl and C₁₋₄alkoxy; and the remaining variables are as defined in any one of the first to twenty-first embodiments.

In a twenty-sixth embodiment, the invention provides a compound of any one of the first to twenty-first embodiments or a pharmaceutically acceptable salt thereof, wherein R¹ is selected from the group consisting of C₃₋₆cycloalkyl, —C₁₋₂ alkyl-C₃₋₆cycloalkyl, a fully saturated 4 to 7 membered heterocycle containing 1 to 2 heteroatoms independently selected from nitrogen, sulfur and oxygen, —C₁₋₂ alkyl-C₄₋₇ heterocycle, wherein the C₄₋₇ heterocycle may be fully or partially saturated and contains 1 to 2 heteroatoms independently selected from nitrogen, sulfur and oxygen, a fully saturated 5 to 8 membered bridged-carbocyclic ring, a fully saturated 5 to 8 membered bridged-heterocyclic ring system having 1 to 2 heteroatoms independently selected from nitrogen and oxygen, a 5 to 10 membered fused heterobicyclic ring system having 1 to 2 heteroatoms independently selected from nitrogen and oxygen and a 5 to 10 membered spiro heterobicyclic ring system having 1 to 2 heteroatoms independently selected from nitrogen and oxygen, wherein R¹ may be optionally substituted with 1, 2 or 3 substituents R^{1a} which are independently selected from halo, nitrile, oxo, halo-substituted C₁₋₄ alkyl, hydroxy-substituted C₁₋₄ alkyl, C₁₋₄ alkyl, C₄₋₇ heterocycle containing 1 to 2 heteroatoms independently selected from nitrogen and oxygen, C₁₋₄ alkyl-O—C₁₋₂ alkyl, hydroxyl and C₁₋₄ alkoxy; and the remaining variables are as defined in any one of the first to twenty-first embodiments.

In a twenty-seventh embodiment, the invention provide a compound of any one of the first to twenty-first embodiments or a pharmaceutically acceptable salt thereof, wherein R¹ is a 5 to 8 membered bridged-heterocyclic ring system which contains 1 to 2 heteroatoms independently selected from nitrogen and oxygen, wherein the 5 to 8 membered bridged-heterocyclic ring system is optionally substituted with one or two substituents R^{1a} independently selected from C₁₋₄alkyl, halogen, halo-substituted C₁₋₄ alkyl, hydroxyl and C₁₋₄alkoxy; and the remaining variables are as defined in any one of the first to twenty-first embodiments. In one embodiment, le is a 5 to 8 membered bridged-heterocyclic ring system containing one oxygen atom, wherein the 5 to 8 membered bridged-heterocyclic ring is optionally substituted with one or two substituents R^{1a} independently selected from C₁₋₄alkyl, halogen, halo-substituted C₁₋₄ alkyl, hydroxyl and C₁₋₄alkoxy; and the remaining variables are as defined in the twenty-seventh embodiment. In one embodiment, le is a 5 to 8 membered bridged-heterocyclic ring system selected from the group consisting of 3-oxabicyclo[3.1.0]hexane, 2-oxabicyclo[2.1.1]hexane, 3-oxabicyclo[2.1.1]hexane, 3-oxabicyclo[4.1.0]heptane, 2-oxabicyclo[2.2.1]heptane, 2-oxabicyclo[2.2.1]heptane, 2-oxabicyclo[3.1.1]heptane, 2-oxabicyclo[2.2.2]octane, 8-oxabicyclo[3.2.1]octane, and 2,6-dioxabicyclo[3.2.1]octane, wherein the 5 to 8 membered bridged-heterocyclic ring is optionally substituted with one or two substituents R^{1a} independently selected from C₁₋₄alkyl, halogen, halo-substituted C₁₋₄ alkyl, hydroxyl and C₁₋₄alkoxy; and the remaining variables are as defined in the twenty-seventh embodiment.

In a twenty-eighth embodiment, the invention provides a compound of any one of the first to twenty-first embodiments or a pharmaceutically acceptable salt thereof, wherein R¹ is a 5 to 8 membered bridged-heterocyclic ring system represented by the following formula:

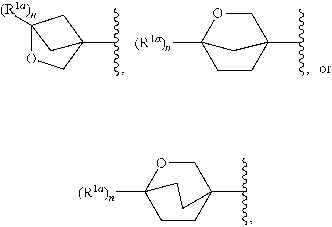

wherein R^{1a} is C₁₋₄ alkyl or halo-substituted C₁₋₄ alkyl; and n is 0 or 1; and the remaining variables are as defined in the twenty-seventh embodiment. In one embodiment, R^{1a} is CH₃ or CH₂F.

In a twenty-ninth embodiment, the invention provide a compound of any one of the first to twenty-first embodiments or a pharmaceutically acceptable salt thereof, wherein R¹ is selected from the group consisting of H, Cl, trifluoromethyl, 1,1-difluoroethyl, 1-cyano-1-methyl-ethyl, 2-cyanopropyl, 3-methoxypropyl, 1-cyano-2-methylpropan-2-yl, t-butyl, cyclopropyl, 1-methoxycyclopropyl, 2-fluorocyclopropyl, (1R,2S)-2-fluorocyclopropyl, (1S,2R)-2-fluorocyclopropyl, (1R,2R)-2-fluorocyclopropyl, (1S,2S)-2-fluorocyclopropyl, 2,2-difluorocyclopropyl, (1R)-2,2-difluorocyclopropyl, (1S)-2,2-difluorocyclopropyl, 3-methoxycyclobutyl, 3-methoxycyclopentyl, bicyclo[1.1.1]pentan-1-yl, 3-cyanobicyclo[1.1.1]pentan-1-yl, 3-methoxybicyclo[1.1.1]pentan-1-yl, 3-fluoro-1-bicyclo[1.1.1]pentanyl, 3-(difluoromethyl)bicyclo[1.1.1]pentan-1-yl, tetrahydrofuran-3-yl, tetrahydrofuran-3-yl, (S)-tetrahydrofuran-3-yl, (R)-tetrahydrofuran-3-yl, (tetrahydrofuran-3-yl)methyl, (S)-(tetrahydrofuran-3-yl)methyl, (R)-(tetrahydrofuran-3-yl)methyl, tetrahydro-2H-pyran-3-yl, (S)-tetrahydro-2H-pyran-3-yl, (R)-tetrahydro-2H-pyran-3-yl, tetrahydro-2H-pyran-4-yl, 2,2-dimethyltetrahydro-2H-pyran-4-yl, (tetrahydro-2H-pyran-4-yl)methyl, 1,4-dioxan-2-yl, (1,4-dioxan-2-yl)methyl, 3-oxabicyclo[3.1.0]hexan-6-yl, (I S,5R)-3-oxabicyclo[3.1.0]hexan-1-yl, 2-oxabicyclo[2.1.1]hexan-4-yl, 1-methyl-2-oxabicyclo[2.1.1]hexan-4-yl, 1-methyl-3-oxabicyclo[2.1.1]hexan-4-yl, 1-(fluoromethyl)-2-oxabicyclo[2.1.1]hexan-4-yl, 1,3,3-trimethyl-2-oxabicyclo[2.1.1]hexan-4-yl, 3-oxabicyclo[4.1.0]heptan-7-yl, 2-oxabicyclo[2.2.1]heptan-4-yl, 2-oxabicyclo[2.2.1]heptan-4-yl, 2-oxabicyclo[2.2.1]heptan-4-yl, (1S,4R)-2-oxabicyclo[2.2.1]heptan-4-yl, (1R,4S)-2-oxabicyclo[2.2.1]heptan-4-yl, 1-methyl-2-oxabicyclo[2.2.1]heptan-4-yl, (1R,4S)-1-methyl-2-oxabicyclo[2.2.1]heptan-4-yl, (1S,4R)-1-methyl-2-oxabicyclo[2.2.1]heptan-4-yl, 1-methyl-2-oxabicyclo[2.2.1]heptan-4-yl, 1-methyl-2-oxabicyclo[2.2.1]heptan-4-yl, 1-methyl-2-oxabicyclo[3.1.1]heptan-5-yl, 5-oxaspiro[2.4]heptan-1-yl, 1-methyl-2-oxabicyclo[2.2.2]octan-4-yl, 4-methyl-2-oxabicyclo[2.2.2]octan-1-yl, 8-oxabicyclo[3.2.1]octan-3-yl, 4-oxaspiro[2.5]octan-1-yl, 6-oxaspiro[2.5]octan-2-yl, 6-oxaspiro[3.4]octan-2-yl, 2,6-dioxabicyclo[3.2.1]octan-1-yl, (1R,5R)-2,6-dioxabicyclo[3.2.1]octan-1-yl, 4-methyl-3-oxaspiro[bicyclo[2.1.1]hexane-2,3′-oxetan]-1-yl, and 1-(2,2-difluoroethyl)azetidin-3-yl; and the remaining variables are as defined in any one of the first to twenty-first embodiments.

In a thirtieth embodiment of the invention provides a compound of formula (I′), (I), (Ia), (Ib), (Ic) or (Id) of any one of embodiments one to eight or a pharmaceutically acceptable salt thereof, wherein:

R¹ is a C₁₋₅ alkyl which is optionally substituted with 1 or 3 substituents independently selected from the group consisting of halogen, halo-substituted C₁₋₄ alkyl, hydroxyl, C₁₋₄alkoxy and C₃₋₆cycloalkyl, wherein said C₃₋₆cycloalkyl is optionally substituted with 1 or 2 substituents independently selected from the group consisting of halogen, halo-substituted C₁₋₄ alkyl, hydroxyl and C₁₋₄alkoxy; and

R³ is pyridinyl optionally substituted with 1 or 2 substituents independently selected from and C₁₋₄ alkyl and halo-substituted C₁₋₄ alkyl; and the remaining variables are as defined in the first, second, third, fourth, fifth, sixth, seventh or eighth embodiment.

In a thirty-first embodiment of the invention provides a compound of formula (I′), (I), (Ia), (Ib), (Ic) or (Id) of any of one of embodiments one to eight or a pharmaceutically acceptable salt thereof, wherein:

R¹ is a fully saturated C₄₋₇ heterocycle or a 5 to 8 membered bridged-heterocyclic ring system which contain 1 to 2 heteroatoms independently selected from nitrogen and oxygen, said C₄₋₇ heterocycle or a 5 to 8 membered bridged-heterocyclic ring system may be optionally substituted with 1 or 2 substituents independently selected from the group consisting of C₁₋₄alkyl, halogen, halo-substituted C₁₋₄ alkyl, hydroxyl and C₁₋₄alkoxy; and

R³ is pyridinyl optionally substituted with 1 or 2 substituents independently selected from and C₁₋₄ alkyl and halo-substituted C₁₋₄ alkyl; and the remaining variables are as defined in the first, second, third, fourth, fifth, sixth, seventh eighth embodiment.

In a thirty-second embodiment, the invention provides a compound of any one of the first to thirty-first embodiment, or a pharmaceutically acceptable salt thereof, wherein:

R⁶ is an optionally substituted C₁₋₅alkyl or an optionally substituted C₃₋₆cycloalkyl, wherein the C₁₋₅alkyl is optionally substituted with 1 to 3 substituents independently selected from halogen, hydroxyl and C₁₋₄alkoxy and the C₃₋₆cycloalkyl is optionally substituted with 1 to 3 substituents independently selected from halo, C₁₋₄alky, halo-substituted C₁₋₄ alkyl and C₁₋₄alkoxy; and the remaining variables are as defined in any one of the first to thirty-first embodiments.

In a thirty-third embodiment, the invention provides a compound of any one of the first to thirty-second embodiment, or a pharmaceutically acceptable salt thereof, wherein:

R⁶ is selected from the group consisting of methyl, ethyl, 2-(difluoromethoxy)ethyl, difluoromethyl, 2-fluoroethyl, 2,2-difluoroethyl, propyl, isopropyl, 1,1,1-trifluoropropan-2-yl), (R)-1,1,1-trifluoropropan-2-yl), (S)-1,1,1-trifluoropropan-2-yl), sec-butyl, (R)-sec-butyl, (S)-sec-butyl, isobutyl, cyclopropylmethyl, cyclobutyl, 3-methylcyclobutyl, 3-(difluoromethyl)cyclobutyl, 3,3-difluorocyclobutyl, 3,3-dimethylcyclobutyl, 2,2-dimethylcyclobutyl, 3-ethoxycyclobutyl, cyclopentyl, spiro[2.3]hexan-5-yl, oxetan-3-yl, 4-oxaspiro[2.4]heptan-6-yl, tetrahydrofuran-3-yl, (R)-tetrahydrofuran-3-yl, (S)-tetrahydrofuran-3-yl, 5,5-dimethyltetrahydrofuran-3-yl, tetrahydro-2H-pyran-3-yl, (R)-tetrahydro-2H-pyran-3-yl, (S)-tetrahydro-2H-pyran-3-yl, tetrahydro-2H-pyran-4-yl, (7-oxabicyclo[2.2.1]heptan-2-yl)methyl, (3-methyltetrahydrofuran-3-yl)methyl, (4-fluorotetrahydro-2H-pyran-4-yl)methyl, (3,3-difluorocyclobutyl)methyl, (2,2-difluorocyclopropyl)methyl, 1-methyl-2-oxopyrrolidin-3-yl, and 2-(tetrahydrofuran-3-yl)ethyl; and the remaining variables are as defined in any one of the first to thirty-second embodiments.

In a thirty-fourth embodiment, the invention provides a compound of the first or second embodiment, wherein the compound is represented by formula (Ia), (Ib), (Ic) or (Id) or a pharmaceutically acceptable salt thereof, wherein:

R¹ is a 5 to 8 membered bridged-heterocyclic ring system which contains 1 to 2 heteroatoms independently selected from nitrogen and oxygen, wherein the 5 to 8 membered bridged-heterocyclic ring system is optionally substituted with one or two substituents R^{1a};

R^{1a}, for each occurrence, is independently selected from C₁₋₄alkyl, halogen, halo-substituted C₁₋₄ alkyl, hydroxyl and C₁₋₄alkoxy;

R³ is a 5 or 6 membered monocyclic heteroaryl having 1 to 2 heteroatoms independently selected from nitrogen and oxygen, pyridinyl-2(1H)-one or a 9 to 10 membered bicyclic heteroaryl having 1 to 3 heteroatoms independently selected from nitrogen and oxygen, wherein the monocyclic heteroaryl, pyridinyl-2(1H)-one or the bicyclic heteroaryl are each optionally substituted with 1 or 2 R⁴;

R⁴, for each occurrence, is independently selected from hydroxyl, halo, halo-substituted C₁₋₄ alkyl, —NR⁸R⁹, and C₁₋₄ alkyl;

R⁵ is OR⁶; and

R⁶ is an optionally substituted C₁₋₅alkyl or an optionally substituted C₃₋₆cycloalkyl, wherein the C₁₋₅alkyl is optionally substituted with 1 to 3 substituents independently selected from halogen, hydroxyl and C₁₋₄alkoxy and the C₃₋₆cycloalkyl is optionally substituted with 1 to 3 substituents independently selected from halo, C₁₋₄alky, halo-substituted C₁₋₄ alkyl and C₁₋₄alkoxy.

In one embodiment, the compound is represented by formula (Ic) or (Id).

In a thirty-fifth embodiment, the invention provides a compound of the thirty-fourth embodiment, or a pharmaceutically acceptable salt thereof, wherein:

R¹ is a 5 to 8 membered bridged-heterocyclic ring system containing one oxygen atom, wherein the 5 to 8 membered bridged-heterocyclic ring system is optionally substituted with one substituent R^{1a};

R^{1a} is C₁₋₄alkyl or halo-substituted C₁₋₄ alkyl; R³ is a 5 or 6 membered monocyclic heteroaryl having 1 to 2 nitrogen atoms, pyridinyl-2(1H)-one or a 9 to 10 membered bicyclic heteroaryl having 2 to 3 nitrogen atoms, wherein the monocyclic heteroaryl, pyridinyl-2(1H)-one or the bicyclic heteroaryl are each optionally substituted with 1 or 2 R⁴;

R⁴, for each occurrence, is independently selected from hydroxyl, halo-substituted C₁₋₄ alkyl, and C₁₋₄ alkyl;

R⁵ is OR⁶; and

R⁶ is an optionally substituted C₁₋₅alkyl or an optionally substituted C₃₋₆cycloalkyl, wherein the C₁₋₅alkyl is optionally substituted with 1 to 3 substituents independently selected from halogen and the C₃₋₆cycloalkyl is optionally substituted with 1 to 3 substituents independently selected from C₁₋₄alkyl, halo-substituted C₁₋₄alkyl and halogen.

In one embodiment, for compounds of the thirty-fifth embodiment or a pharmaceutically acceptable salt thereof, the 5 to 8 membered bridged-heterocyclic ring system represented by R¹ is selected from the group consisting of 3-oxabicyclo[3.1.0]hexane, 2-oxabicyclo[2.1.1]hexane, 3-oxabicyclo[2.1.1]hexane, 3-oxabicyclo[4.1.0]heptane, 2-oxabicyclo[2.2.1]heptane, 2-oxabicyclo[2.2.1]heptane, 2-oxabicyclo[3.1.1]heptane, 2-oxabicyclo[2.2.2]octane, 8-oxabicyclo[3.2.1]octane, and 2,6-dioxabicyclo[3.2.1]octane, wherein the 5 to 8 membered bridged-heterocyclic ring is optionally substituted with one substituent R^{1a}; and the remaining variables are as defined in the thirty-fifth embodiment.

In a thirty-sixth embodiment, the invention provides a compound of the thirty-fifth embodiment, or a pharmaceutically acceptable salt thereof, wherein:

R¹ is

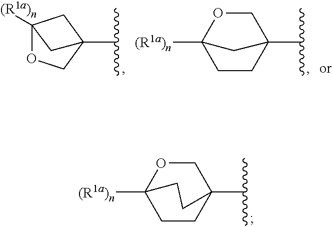

R^{1a} is C₁₋₄ alkyl or halo-substituted C₁₋₄ alkyl;

n is 0 or 1;

R³ is

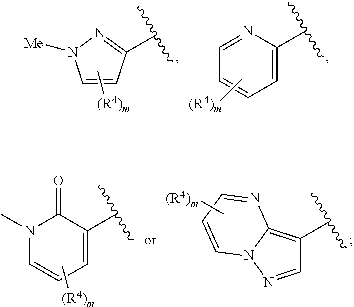

R⁴ is hydroxyl, C₁₋₄ alkyl or halo-substituted C₁₋₄ alkyl;

m is 0, 1 or 2;

R⁵ is OR⁶; and

R⁶ is C₁₋₄alkyl or C₄₋₆cycloalkyl.

In a thirty-seventh embodiment, the invention provides a compound of the thirty-sixth embodiment, or a pharmaceutically acceptable salt thereof, wherein:

R^{1a} is CH₃ or CH₂F; and R⁴ is CH₃, CHF₂ or OH; R⁶ is —CH(CH₃)₂, cyclobutyl, or cyclopentyl; and the remaining variables are as defined in the thirty-sixth embodiment.

In a thirty-eighth embodiment, the invention provides a compound of formula (I′), (I), (Ia), (Ib), (Ic) or (Id), or a pharmaceutically acceptable salt thereof, wherein:

R¹ is a fully saturated C₄₋₇ heterocycle or a fully saturated 5 to 8 membered bridged-heterocyclic ring system which contain 1 to 2 heteroatoms independently selected from nitrogen and oxygen, said C₄₋₇ heterocycle or said 5 to 8 membered bridged-heterocyclic ring system is optionally substituted with 1 or 2 substituents independently selected from the group consisting of C₁₋₄alkyl, halogen, halo-substituted C₁₋₄ alkyl, hydroxyl and C₁₋₄alkoxy;

R³ is phenyl, 5 or 6 membered monocyclic heteroaryl having 1 to 3 heteroatoms independently selected from nitrogen and oxygen, pyridinyl-2(1H)-one, pyrimidin-4(3H)-one or a 9 to 10 membered bicyclic heteroaryl having 1 to 3 heteroatoms independently selected from nitrogen and oxygen, wherein the monocyclic heteroaryl, pyridinyl-2(1H)-one, pyrimidin-4(3H)-one or the bicyclic heteroaryl are each optionally substituted with 1 or 2 R⁴;

R⁴, for each occurrence, is independently selected from hydroxyl, halo, halo-substituted C₁₋₄ alkyl, —NR⁸R⁹, C₁₋₄alkoxy, C₃₋₆cycloalkyl, and C₁₋₄ alkyl;

R⁵ is OR⁶; and

R⁶ is an optionally substituted C₁₋₅alkyl or an optionally substituted C₃₋₆cycloalkyl, wherein the C₁₋₅alkyl is optionally substituted with 1 to 3 substituents independently selected from halogen, hydroxyl and C₁₋₄alkoxy and the C₃₋₆cycloalkyl is optionally substituted with 1 to 3 substituents independently selected from halo, C₁₋₄alky, halo-substituted C₁₋₄ alkyl and C₁₋₄alkoxy.

In a thirty-ninth embodiment, the invention provides a compound of the thirty-eighth embodiment or a pharmaceutically acceptable salt thereof, wherein:

R¹ a fully saturated C₄₋₇ heterocycle selected from the group consisting of tetrahydrofuran, tetrahydropyran, and 1,4-dioxane or a fully saturated 5 to 8 membered bridged-heterocyclic ring system selected from the group consisting of 3-oxabicyclo[3.1.0]hexane, 2-oxabicyclo[2.1.1]hexane, 3-oxabicyclo[2.1.1]hexane, 3-oxabicyclo[4.1.0]heptane, 2-oxabicyclo[2.2.1]heptane, 2-oxabicyclo[2.2.1]heptane, 2-oxabicyclo[3.1.1]heptane, 2-oxabicyclo[2.2.2]octane, 8-oxabicyclo[3.2.1]octane, and 2,6-dioxabicyclo[3.2.1]octane, wherein the C₄₋₇ heterocycle or the 5 to 8 membered bridged-heterocyclic ring system is optionally substituted with 1 or 2 substituents independently selected from the group consisting of C₁₋₄alkyl, halogen, halo-substituted C₁₋₄ alkyl, hydroxyl and C₁₋₄alkoxy;

R³ is phenyl, 5 or 6 membered monocyclic heteroaryl selected from the group consisting of pyridine, pyrimidine, 2H-1,2,3-triazole, isoxazole, isothiazole, thiazole, pyrazole and thiophene, pyridinyl-2(1H)-one, pyrimidin-4(3H)-one, or a 9 to 10 membered bicyclic heteroaryl selected from pyrazolo[1,5-a]pyridine, [1,2,4]triazolo[4,3-a]pyridine, isothiazolo[4,3-b]pyridine, pyrazolo[1,5-a]pyrimidine, pyrido[3,2-d]pyrimidine, imidazo[1,2-b]pyridazine, thieno[2,3-b]pyrazine, 1H-benzo[d]imidazole, benzo[d]thiazole, 1,6-naphthyridine, 1,5-naphthyridine, and 2H-indazole, wherein the monocyclic heteroaryl, pyridinyl-2(1H)-one, pyrimidin-4(3H)-one or the bicyclic heteroaryl are each optionally substituted with 1 or 2 R⁴; and the remaining variables are as defined above in the thirty-eighth embodiment.

In a fortieth embodiment, the invention provides a compound described herein (e.g., a compound of any one of Examples 1-658) or a pharmaceutically acceptable salt thereof.

In a forty-first embodiment of the invention provides a compound according embodiment one, selected from the group consisting of:
7-methoxy-2-(1-methyl-2-oxabicyclo[2.1.1]hexan-4-yl)-N-(2-pyridyl)imidazo[1,2-a]pyridine-6-carboxamide;
7-methoxy-2-(3-methoxy-1-bicyclo[1.1.1]pentanyl)-N-(2-pyridyl)imidazo[1,2-a]pyridine-6-carboxamide;
2-(3-fluoro-1-bicyclo[1.1.1]pentanyl)-7-methoxy-N-(2-pyridyl)imidazo[1,2-a]pyridine-6-carboxamide;
7-methoxy-2-(1-methyl-3-oxabicyclo[2.1.1]hexan-4-yl)-N-(2-pyridyl)imidazo[1,2-a]pyridine-6-carboxamide;
2-[4-(fluoromethyl)-3-oxabicyclo[2.1.1]hexan-1-yl]-7-methoxy-N-(2-pyridyl)imidazo[1,2-a]pyridine-6-carboxamide;
7-methoxy-2-(8-oxaspiro[2.5]octan-2-yl)-N-(2-pyridyl)imidazo[1,2-a]pyridine-6-carboxamide;
2-(1-(fluoromethyl)-2-oxabicyclo[2.1.1]hexan-4-yl)-7-methoxy-N-(6-methoxypyridin-2-yl)imidazo[1,2-a]pyridine-6-carboxamide;
7-methoxy-2-(3-methoxycyclobutyl)-N-(6-methoxypyridin-2-yl)imidazo[1,2-a]pyridine-6-carboxamide;
7-methoxy-N-(6-methoxypyridin-2-yl)-2-(4-oxaspiro[2.5]octan-1-yl)imidazo[1,2-a]pyridine-6-carboxamide;
7-methoxy-N-(6-methoxypyridin-2-yl)-2-((tetrahydro-2H-pyran-4-yl)methyl)imidazo[1,2-a]pyridine-6-carboxamide;
7-methoxy-N-(6-methoxypyridin-2-yl)-2-(6-oxaspiro[3.4]octan-2-yl)imidazo[1,2-a]pyridine-6-carboxamide;
2-(2-cyanopropyl)-7-methoxy-N-(6-methoxypyridin-2-yl)imidazo[1,2-a]pyridine-6-carboxamide;
2-(1-cyano-2-methylpropan-2-yl)-7-methoxy-N-(6-methoxypyridin-2-yl)imidazo[1,2-a]pyridine-6-carboxamide;
7-methoxy-2-(1-methoxycyclopropyl)-N-(6-methoxypyridin-2-yl)imidazo[1,2-a]pyridine-6-carboxamide;
2-(1,4-dioxan-2-yl)methyl)-7-methoxy-N-(6-methoxypyridin-2-yl)imidazo[1,2-a]pyridine-6-carboxamide;
7-methoxy-N-(6-methoxypyridin-2-yl)-2-(5-oxaspiro[2.4]heptan-1-yl)imidazo[1,2-a]pyridine-6-carboxamide;
7-methoxy-2-(3-methoxycyclopentyl)-N-(6-methoxypyridin-2-yl)imidazo[1,2-a]pyridine-6-carboxamide;
2-(8-oxabicyclo[3.2.1]octan-3-yl)-7-methoxy-N-(6-methoxypyridin-2-yl)imidazo[1,2-a]pyridine-6-carboxamide;
2-(3-cyanobicyclo[1.1.1]pentan-1-yl)-7-methoxy-N-(6-methoxypyridin-2-yl)imidazo[1,2-a]pyridine-6-carboxamide;
2-(3-(difluoromethyl)bicyclo[1.1.1]pentan-1-yl)-7-methoxy-N-(6-methoxypyridin-2-yl)imidazo[1,2-a]pyridine-6-carboxamide;
7-methoxy-N-(6-methoxypyridin-2-yl)-2-(tetrahydro-2H-pyran-4-yl)imidazo[1,2-a]pyridine-6-carboxamide;
2-(3-oxabicyclo[3.1.0]hexan-6-yl)-7-methoxy-N-(6-methoxypyridin-2-yl)imidazo[1,2-a]pyridine-6-carboxamide;
7-methoxy-N-(6-methoxypyridin-2-yl)-2-(tetrahydrofuran-3-yl)imidazo[1,2-a]pyridine-6-carboxamide;
7-methoxy-N-(6-methoxypyridin-2-yl)-2-((tetrahydrofuran-3-yl)methyl)imidazo[1,2-a]pyridine-6-carboxamide;
2-(3-oxabicyclo[4.1.0]heptan-7-yl)-7-methoxy-N-(6-methoxypyridin-2-yl)imidazo[1,2-a]pyridine-6-carboxamide;
7-methoxy-N-(6-methoxypyridin-2-yl)-2-(4-methyl-2-oxabicyclo[2.1.1]hexan-1-yl)imidazo[1,2-a]pyridine-6-carboxamide;
7-methoxy-N-(6-methoxypyridin-2-yl)-2-(1-methyl-2-oxabicyclo[2.2.1]heptan-4-yl)imidazo[1,2-a]pyridine-6-carboxamide;
7-methoxy-N-(6-methoxypyridin-2-yl)-2-(4-methyl-3-oxaspiro[bicyclo[2.1.1]hexane-2,3′-oxetan]-1-yl)imidazo[1,2-a]pyridine-6-carboxamide;
7-methoxy-N-(6-methoxypyridin-2-yl)-2-(1-methyl-2-oxabicyclo[2.2.2]octan-4-yl)imidazo[1,2-a]pyridine-6-carboxamide;
Rac-2-((1S,5R)-3-oxabicyclo[3.1.0]hexan-1-yl)-7-methoxy-N-(6-methoxypyridin-2-yl)imidazo[1,2-a]pyridine-6-carboxamide;
7-methoxy-N-(6-methoxypyridin-2-yl)-2-(1-methyl-2-oxabicyclo[2.1.1]hexan-4-yl)imidazo[1,2-a]pyridine-6-carboxamide;
2-(3-oxabicyclo[3.1.0]hexan-6-yl)-7-methoxy-N-(6-(trifluoromethyl)pyridin-2-yl)imidazo[1,2-a]pyridine-6-carboxamide;
7-methoxy-2-(tetrahydrofuran-3-ylmethyl)-N-[6-(trifluoromethyl)-2-pyridyl]imidazo[1,2-a]pyridine-6-carboxamide;
7-methoxy-2-(1-methoxycyclopropyl)-N-(6-(trifluoromethyl)pyridin-2-yl)imidazo[1,2-a]pyridine-6-carboxamide;
7-methoxy-2-(tetrahydrofuran-3-yl)-N-(6-(trifluoromethyl)pyridin-2-yl)imidazo[1,2-a]pyridine-6-carboxamide;
7-methoxy-2-(3-methoxycyclobutyl)-N-(6-(trifluoromethyl)pyridin-2-yl)imidazo[1,2-a]pyridine-6-carboxamide;
2-(3-cyanobicyclo[1.1.1]pentan-1-yl)-7-methoxy-N-(6-(trifluoromethyl)pyridin-2-yl)imidazo[1,2-a]pyridine-6-carboxamide;
7-methoxy-2-(6-oxaspiro[3.4]octan-2-yl)-N-(6-(trifluoromethyl)pyridin-2-yl)imidazo[1,2-a]pyridine-6-carboxamide;
7-methoxy-2-(5-oxaspiro[2.4]heptan-1-yl)-N-(6-(trifluoromethyl)pyridin-2-yl)imidazo[1,2-a]pyridine-6-carboxamide;
7-methoxy-2-(6-oxaspiro[2.5]octan-2-yl)-n-[6-(trifluoromethyl)-2-pyridyl]imidazo[1,2-a]pyridine-6-carboxamide;
7-methoxy-2-(3-methoxycyclopentyl)-N-(6-(trifluoromethyl)pyridin-2-yl)imidazo[1,2-a]pyridine-6-carboxamide;
2-(2-cyanopropyl)-7-methoxy-N-(6-(trifluoromethyl)pyridin-2-yl)imidazo[1,2-a]pyridine-6-carboxamide;
2-(2,2-dimethyltetrahydro-2H-pyran-4-yl)-7-methoxy-N-(6-(trifluoromethyl)pyridin-2-yl)imidazo[1,2-a]pyridine-6-carboxamide;
2-(8-oxabicyclo[3.2.1]octan-3-yl)-7-methoxy-N-(6-(trifluoromethyl)pyridin-2-yl)imidazo[1,2-a]pyridine-6-carboxamide;
7-methoxy-2-((tetrahydro-2H-pyran-4-yl)methyl)-N-(6-(trifluoromethyl)pyridin-2-yl)imidazo[1,2-a]pyridine-6-carboxamide;
2-(1-cyano-2-methylpropan-2-yl)-7-methoxy-N-(6-(trifluoromethyl)pyridin-2-yl)imidazo[1,2-a]pyridine-6-carboxamide;
N-(6-methoxypyridin-2-yl)-2-(tetrahydro-2H-pyran-4-yl)imidazo[1,2-a]pyridine-6-carboxamide;
N-(1-(difluoromethyl)-1H-pyrazol-3-yl)-7-methoxy-2-(tetrahydro-2H-pyran-4-yl)imidazo[1,2-a]pyridine-6-carboxamide;
N-(1-(difluoromethyl)-1H-pyrazol-3-yl)-2-(tetrahydro-2H-pyran-4-yl)imidazo[1,2-a]pyridine-6-carboxamide;
(S)-7-methoxy-N-(6-methoxypyridin-2-yl)-2-((tetrahydrofuran-3-yl)methyl)imidazo[1,2-a]pyridine-6-carboxamide;
(R)-7-methoxy-N-(6-methoxypyridin-2-yl)-2-((tetrahydrofuran-3-yl)methyl)imidazo[1,2-a]pyridine-6-carboxamide;
N-(6-ethylpyridin-2-yl)-2-(1-(fluoromethyl)-2-oxabicyclo[2.1.1]hexan-4-yl)-7-methoxyimidazo[1,2-a]pyridine-6-carboxamide;
N-(6-ethylpyridin-2-yl)-7-methoxy-2-(tetrahydro-2H-pyran-4-yl)imidazo[1,2-a]pyridine-6-carboxamide;
N-(6-ethylpyridin-2-yl)-7-methoxy-2-(4-oxaspiro[2.5]octan-1-yl)imidazo[1,2-a]pyridine-6-carboxamide;
N-(6-ethylpyridin-2-yl)-7-methoxy-2-(3-methoxycyclobutyl)imidazo[1,2-a]pyridine-6-carboxamide;
N-(6-ethylpyridin-2-yl)-7-methoxy-2-(6-oxaspiro[3.4]octan-2-yl)imidazo[1,2-a]pyridine-6-carboxamide;
N-[6-(difluoromethyl)-2-pyridyl]-7-ethoxy-2-[[(3 S)-tetrahydrofuran-3-yl]methyl]imidazo[1,2-a]pyridine-6-carboxamide;
N-[6-(difluoromethyl)-2-pyridyl]-7-ethoxy-2-[[(3R)-tetrahydrofuran-3-yl]methyl]imidazo[1,2-a]pyridine-6-carboxamide;
7-ethoxy-2-[(1R,2 S)-2-fluorocyclopropyl]-N-(6-methoxy-2-pyridyl)imidazo[1,2-a]pyridine-6-carboxamide;
7-ethoxy-2-[(1 S,2R)-2-fluorocyclopropyl]-N-(6-methoxy-2-pyridyl)imidazo[1,2-a]pyridine-6-carboxamide;
2-[(1R)-2,2-difluorocyclopropyl]-7-ethoxy-N-(6-methoxy-2-pyridyl)imidazo[1,2-a]pyridine-6-carboxamide;
2-[(1 S)-2,2-difluorocyclopropyl]-7-ethoxy-N-(6-methoxy-2-pyridyl)imidazo[1,2-a]pyridine-6-carboxamide;
(R)-8-methoxy-2-((tetrahydrofuran-3-yl)methyl)-N-(6-(trifluoromethyl)pyridin-2-yl)imidazo[1,2-a]pyrazine-6-carboxamide;
(S)-8-methoxy-2-((tetrahydrofuran-3-yl)methyl)-N-(6-(trifluoromethyl)pyridin-2-yl)imidazo[1,2-a]pyrazine-6-carboxamide;
8-methoxy-2-((tetrahydrofuran-3-yl)methyl)-N-(6-(trifluoromethyl)pyridin-2-yl)imidazo[1,2-a]pyridine-6-carboxamide;
N-[6-(difluoromethyl)-2-pyridyl]-7-ethoxy-2-tetrahydropyran-4-yl-imidazo[1,2-a]pyridine-6-carboxamide;
2-(1-cyano-1-methyl-ethyl)-N-[6-(difluoromethyl)-2-pyridyl]-7-ethoxy-imidazo[1,2-a]pyridine-6-carboxamide;
8-methoxy-2-tetrahydropyran-4-yl-N-[6-(trifluoromethyl)-2-pyridyl]imidazo[1,2-a]pyridine-6-carboxamide;
8-methoxy-2-tetrahydropyran-4-yl-N-[6-(trifluoromethyl)-2-pyridyl]imidazo[1,2-a]pyrazine-6-carboxamide;
8-methoxy-N-(6-methoxy-2-pyridyl)-2-tetrahydropyran-4-yl-imidazo[1,2-a]pyrazine-6-carboxamide;
8-methoxy-N-(2-pyridyl)-2-tetrahydropyran-4-yl-imidazo[1,2-a]pyrazine-6-carboxamide;
7-ethoxy-2-[(1R,2R)-2-fluorocyclopropyl]-N-(6-methoxy-2-pyridyl)imidazo[1,2-a]pyridine-6-carboxamide;
7-ethoxy-2-[(1S,2S)-2-fluorocyclopropyl]-N-(6-methoxy-2-pyridyl)imidazo[1,2-a]pyridine-6-carboxamide;
N-(6-methoxy-2-pyridyl)-2-tetrahydropyran-4-yl-imidazo[1,2-a]pyrazine-6-carboxamide;
2-tetrahydropyran-4-yl-N-[6-(trifluoromethyl)-2-pyridyl]imidazo[1,2-a]pyrazine-6-carboxamide;
N-[6-(difluoromethyl)-2-pyridyl]-2-tetrahydropyran-4-yl-imidazo[1,2-a]pyrazine-6-carboxamide;
N-[1-(2-methoxyethyl)pyrazol-3-yl]-2-(3-oxabicyclo[3.1.0]hexan-6-yl)imidazo[1,2-a]pyrazine-6-carboxamide;
N-(6-ethyl-2-pyridyl)-8-methoxy-2-tetrahydropyran-4-yl-imidazo[1,2-a]pyrazine-6-carboxamide;
N-[1-(difluoromethyl)pyrazol-3-yl]-8-methoxy-2-tetrahydropyran-4-yl-imidazo[1,2-a]pyrazine-6-carboxamide;
8-methoxy-N-(1-methyl-1H-pyrazol-3-yl)-2-(tetrahydro-2H-pyran-4-yl)imidazo[1,2-a]pyrazine-6-carboxamide;
8-methoxy-N-(1-methyl-1H-pyrazol-5-yl)-2-(tetrahydro-2H-pyran-4-yl)imidazo[1,2-a]pyrazine-6-carboxamide;
2-[1-(2,2-difluoroethyl)azetidin-3-yl]-N-[6-(difluoromethyl)-2-pyridyl]-7-ethoxy-imidazo[1,2-a]pyridine-6-carboxamide;
N-(6-(difluoromethyl)pyridin-2-yl)-8-methoxy-2-(1-methoxycyclopropyl)imidazo[1,2-a]pyridine-6-carboxamide;
N-(6-(difluoromethyl)pyridin-2-yl)-7-isopropoxy-2-(tetrahydro-2H-pyran-4-yl)imidazo[1,2-a]pyridine-6-carboxamide;
7-isopropoxy-N-(6-methoxypyridin-2-yl)-2-(tetrahydro-2H-pyran-4-yl)imidazo[1,2-a]pyridine-6-carboxamide;
2-(8-oxabicyclo[3.2.1]octan-3-yl)-7-isopropoxy-N-(6-methoxypyridin-2-yl)imidazo[1,2-a]pyridine-6-carboxamide;
2-(difluoromethyl)-N-(6-(difluoromethyl)pyridin-2-yl)-7-ethoxyimidazo[1,2-a]pyridine-6-carboxamide;
N-(6-(difluoromethyl)pyridin-2-yl)-7-isopropoxy-2-(1-methyl-2-oxabicyclo[2.1.1]hexan-4-yl)imidazo[1,2-a]pyridine-6-carboxamide;
7-isopropoxy-N-(6-methoxypyridin-2-yl)-2-(1-methyl-2-oxabicyclo[2.1.1]hexan-4-yl)imidazo[1,2-a]pyridine-6-carboxamide;
2-(1,1-difluoroethyl)-N-(6-(difluoromethyl)pyridin-2-yl)-7-isopropoxyimidazo[1,2-a]pyridine-6-carboxamide;
2-(difluoromethyl)-N-(6-(difluoromethyl)pyridin-2-yl)-7-isopropoxyimidazo[1,2-a]pyridine-6-carboxamide;
2-(difluoromethyl)-7-isopropoxy-N-(6-methoxypyridin-2-yl)imidazo[1,2-a]pyridine-6-carboxamide;
2-(1,1-difluoroethyl)-7-isopropoxy-N-(6-methoxypyridin-2-yl)imidazo[1,2-a]pyridine-6-carboxamide;
N-(6-(difluoromethyl)pyridin-2-yl)-7-isopropoxy-2-(3-methoxypropyl)imidazo[1,2-a]pyridine-6-carboxamide;
7-isopropoxy-2-(3-methoxypropyl)-N-(6-methoxypyridin-2-yl)imidazo[1,2-a]pyridine-6-carboxamide;
N-(4-ethylthiazol-5-yl)-7-methoxy-2-(tetrahydro-2H-pyran-4-yl)imidazo[1,2-a]pyridine-6-carboxamide;
7-methoxy-N-(4-methylthiazol-5-yl)-2-(tetrahydro-2H-pyran-4-yl)imidazo[1,2-a]pyridine-6-carboxamide;
N-(5-fluoro-2-isopropoxyphenyl)-7-methoxy-2-(tetrahydro-2H-pyran-4-yl)imidazo[1,2-a]pyridine-6-carboxamide;
N-(2,3-dihydrobenzofuran-4-yl)-7-methoxy-2-(tetrahydro-2H-pyran-4-yl)imidazo[1,2-a]pyridine-6-carboxamide;
7-methoxy-N-(3-methylisothiazol-4-yl)-2-(tetrahydro-2H-pyran-4-yl)imidazo[1,2-a]pyridine-6-carboxamide;
N-(4-fluoro-2-isopropoxyphenyl)-7-methoxy-2-(tetrahydro-2H-pyran-4-yl)imidazo[1,2-a]pyridine-6-carboxamide;
N-(2-fluoro-3-methylphenyl)-7-methoxy-2-(tetrahydro-2H-pyran-4-yl)imidazo[1,2-a]pyridine-6-carboxamide;
N-(4-chlorothiophen-3-yl)-7-methoxy-2-(tetrahydro-2H-pyran-4-yl)imidazo[1,2-a]pyridine-6-carboxamide;
N-(5-chloro-2-methoxyphenyl)-7-methoxy-2-(tetrahydro-2H-pyran-4-yl)imidazo[1,2-a]pyridine-6-carboxamide;
N-(2,3-difluorophenyl)-7-methoxy-2-(tetrahydro-2H-pyran-4-yl)imidazo[1,2-a]pyridine-6-carboxamide;
N-(3-chloro-2-fluorophenyl)-7-methoxy-2-(tetrahydro-2H-pyran-4-yl)imidazo[1,2-a]pyridine-6-carboxamide;
N-(2-chloro-3-methylphenyl)-7-methoxy-2-(tetrahydro-2H-pyran-4-yl)imidazo[1,2-a]pyridine-6-carboxamide;
7-methoxy-N-(6-methylpyridin-2-yl)-2-(tetrahydro-2H-pyran-4-yl)imidazo[1,2-a]pyridine-6-carboxamide;
N-(7,7-difluorobicyclo[4.1.0]heptan-2-yl)-7-methoxy-2-(tetrahydro-2H-pyran-4-yl)imidazo[1,2-a]pyridine-6-carboxamide;
7-methoxy-2-(tetrahydro-2H-pyran-4-yl)-N-(5,6,7,8-tetrahydronaphthalen-1-yl)imidazo[1,2-a]pyridine-6-carboxamide;
N-(3,5-dichloro-4-fluorophenyl)-7-methoxy-2-(tetrahydro-2H-pyran-4-yl)imidazo[1,2-a]pyridine-6-carboxamide;
7-methoxy-2-(tetrahydro-2H-pyran-4-yl)-N-(2,3,5-trifluorophenyl)imidazo[1,2-a]pyridine-6-carboxamide;
N-(2,3-dihydro-1H-inden-4-yl)-7-methoxy-2-(tetrahydro-2H-pyran-4-yl)imidazo[1,2-a]pyridine-6-carboxamide;
7-methoxy-2-(tetrahydro-2H-pyran-4-yl)-N-(3-(1,1,2-trifluoroethyl)phenyl)imidazo[1,2-a]pyridine-6-carboxamide;
rac-N-((3R,4S)-4-fluorotetrahydrofuran-3-yl)-7-methoxy-2-(tetrahydro-2H-pyran-4-yl)imidazo[1,2-a]pyridine-6-carboxamide;
7-methoxy-N-(4-oxaspiro[bicyclo[3.2.0]heptane-6,1′-cyclobutan]-7-yl)-2-(tetrahydro-2H-pyran-4-yl)imidazo[1,2-a]pyridine-6-carboxamide;
N-(3-(difluoromethyl)phenyl)-7-methoxy-2-(tetrahydro-2H-pyran-4-yl)imidazo[1,2-a]pyridine-6-carboxamide;
7-methoxy-N-(spiro[2.5]octan-5-yl)-2-(tetrahydro-2H-pyran-4-yl)imidazo[1,2-a]pyridine-6-carboxamide;
N-(4,6-dimethylpyridin-2-yl)-7-methoxy-2-(tetrahydro-2H-pyran-4-yl)imidazo[1,2-a]pyridine-6-carboxamide;
N-(2-ethyl-5-fluoropyridin-3-yl)-7-methoxy-2-(tetrahydro-2H-pyran-4-yl)imidazo[1,2-a]pyridine-6-carboxamide;
N-(3-fluoro-2-methylphenyl)-7-methoxy-2-(tetrahydro-2H-pyran-4-yl)imidazo[1,2-a]pyridine-6-carboxamide;
7-methoxy-N-(1,2,3,4-tetrahydro-1,4-epoxynaphthalen-5-yl)-2-(tetrahydro-2H-pyran-4-yl)imidazo[1,2-a]pyridine-6-carboxamide;
7-methoxy-N-(2-methylpyridin-3-yl)-2-(tetrahydro-2H-pyran-4-yl)imidazo[1,2-a]pyridine-6-carboxamide;
N-(4-fluoropyridin-2-yl)-7-methoxy-2-(tetrahydro-2H-pyran-4-yl)imidazo[1,2-a]pyridine-6-carboxamide;
N-(3,5-dichlorophenyl)-7-methoxy-2-(tetrahydro-2H-pyran-4-yl)imidazo[1,2-a]pyridine-6-carboxamide;
7-methoxy-N-(3-methylcyclobutyl)-2-(tetrahydro-2H-pyran-4-yl)imidazo[1,2-a]pyridine-6-carboxamide;
7-methoxy-N-(4-methylthiophen-3-yl)-2-(tetrahydro-2H-pyran-4-yl)imidazo[1,2-a]pyridine-6-carboxamide;
7-methoxy-N-(3-methoxy-2,3-dihydro-1H-inden-1-yl)-2-(tetrahydro-2H-pyran-4-yl)imidazo[1,2-a]pyridine-6-carboxamide;
7-methoxy-2-tetrahydropyran-4-yl-N-(3,4,5-trifluorophenyl)imidazo[1,2-a]pyridine-6-carboxamide;
N-isothiazol-4-yl-7-methoxy-2-tetrahydropyran-4-yl-imidazo[1,2-a]pyridine-6-carboxamide;
N-(3-fluorocyclohexyl)-7-methoxy-2-tetrahydropyran-4-yl-imidazo[1,2-a]pyridine-6-carboxamide;
N-(2-isobutylphenyl)-7-methoxy-2-tetrahydropyran-4-yl-imidazo[1,2-a]pyridine-6-carboxamide;
N-[3-(fluoromethyl)phenyl]-7-methoxy-2-tetrahydropyran-4-yl-imidazo[1,2-a]pyridine-6-carboxamide;
7-methoxy-N-[2-methyl-3-(trifluoromethyl)phenyl]-2-tetrahydropyran-4-yl-imidazo[1,2-a]pyridine-6-carboxamide;
7-methoxy-2-(tetrahydro-2H-pyran-4-yl)-N-(m-tolyl)imidazo[1,2-a]pyridine-6-carboxamide;
N-(3-chlorophenyl)-7-methoxy-2-(tetrahydro-2H-pyran-4-yl)imidazo[1,2-a]pyridine-6-carboxamide;
N-(3-(1,1-difluoroethyl)phenyl)-7-methoxy-2-(tetrahydro-2H-pyran-4-yl)imidazo[1,2-a]pyridine-6-carboxamide;
7-methoxy-N-(pyridin-2-yl)-2-(tetrahydro-2H-pyran-4-yl)imidazo[1,2-a]pyridine-6-carboxamide;
N-(2-fluorophenyl)-7-methoxy-2-(tetrahydro-2H-pyran-4-yl)imidazo[1,2-a]pyridine-6-carboxamide;
N-(2-cyclopropylpyridin-3-yl)-7-methoxy-2-(tetrahydro-2H-pyran-4-yl)imidazo[1,2-a]pyridine-6-carboxamide;
rac-N-((1R,5R)-bicyclo[3.1.0]hexan-1-yl)-7-methoxy-2-(tetrahydro-2H-pyran-4-yl)imidazo[1,2-a]pyridine-6-carboxamide;
N-(4-isopropylthiazol-5-yl)-7-methoxy-2-(tetrahydro-2H-pyran-4-yl)imidazo[1,2-a]pyridine-6-carboxamide;
N-(3-fluoro-5-methoxyphenyl)-7-methoxy-2-(tetrahydro-2H-pyran-4-yl)imidazo[1,2-a]pyridine-6-carboxamide;
N-(3,5-difluorophenyl)-7-methoxy-2-(tetrahydro-2H-pyran-4-yl)imidazo[1,2-a]pyridine-6-carboxamide;
N-(2,3-dimethylphenyl)-7-methoxy-2-(tetrahydro-2H-pyran-4-yl)imidazo[1,2-a]pyridine-6-carboxamide;
rac-N-((1R,2S)-2-cyclobutylcyclopropyl)-7-methoxy-2-(tetrahydro-2H-pyran-4-yl)imidazo[1,2-a]pyridine-6-carboxamide;
rac-7-methoxy-N-((1R,2R)-2-methoxycyclohexyl)-2-(tetrahydro-2H-pyran-4-yl)imidazo[1,2-a]pyridine-6-carboxamide;
N-(isothiazol-5-yl)-7-methoxy-2-(tetrahydro-2H-pyran-4-yl)imidazo[1,2-a]pyridine-6-carboxamide;
7-methoxy-N-(3-methoxyphenyl)-2-(tetrahydro-2H-pyran-4-yl)imidazo[1,2-a]pyridine-6-carboxamide;
7-methoxy-2-(tetrahydro-2H-pyran-4-yl)-N-(3-(trifluoromethyl)phenyl)imidazo[1,2-a]pyridine-6-carboxamide;
N-(3-(difluoromethyl)-4-fluorophenyl)-7-methoxy-2-(tetrahydro-2H-pyran-4-yl)imidazo[1,2-a]pyridine-6-carboxamide;
7-methoxy-N-(3-methoxy-2-methylphenyl)-2-(tetrahydro-2H-pyran-4-yl)imidazo[1,2-a]pyridine-6-carboxamide;
7-methoxy-N-((1s,4s)-4-methoxycyclohexyl)-2-(tetrahydro-2H-pyran-4-yl)imidazo[1,2-a]pyridine-6-carboxamide;
N-(chroman-8-yl)-7-methoxy-2-(tetrahydro-2H-pyran-4-yl)imidazo[1,2-a]pyridine-6-carboxamide;
N-(3-cyclopropylphenyl)-7-methoxy-2-(tetrahydro-2H-pyran-4-yl)imidazo[1,2-a]pyridine-6-carboxamide;
N-(3-(difluoromethyl)-4,5-difluorophenyl)-7-methoxy-2-(tetrahydro-2H-pyran-4-yl)imidazo[1,2-a]pyridine-6-carboxamide;
N-(3-fluorophenyl)-7-methoxy-2-(tetrahydro-2H-pyran-4-yl)imidazo[1,2-a]pyridine-6-carboxamide;
7-methoxy-N-(2-methoxy-3,5-dimethylphenyl)-2-(tetrahydro-2H-pyran-4-yl)imidazo[1,2-a]pyridine-6-carboxamide;
N-(2-ethylphenyl)-7-methoxy-2-(tetrahydro-2H-pyran-4-yl)imidazo[1,2-a]pyridine-6-carboxamide;
N-(2-isopropoxyphenyl)-7-methoxy-2-(tetrahydro-2H-pyran-4-yl)imidazo[1,2-a]pyridine-6-carboxamide;
N-(3-(difluoromethyl)-5-fluorophenyl)-7-methoxy-2-(tetrahydro-2H-pyran-4-yl)imidazo[1,2-a]pyridine-6-carboxamide;
N-(3-chloro-2-methoxyphenyl)-7-methoxy-2-(tetrahydro-2H-pyran-4-yl)imidazo[1,2-a]pyridine-6-carboxamide;
N-(5-chloro-4-methylthiazol-2-yl)-7-methoxy-2-(tetrahydro-2H-pyran-4-yl)imidazo[1,2-a]pyridine-6-carboxamide;
N-(3-chloro-5-fluorophenyl)-7-methoxy-2-(tetrahydro-2H-pyran-4-yl)imidazo[1,2-a]pyridine-6-carboxamide;
N-(2-chloro-3-fluorophenyl)-7-methoxy-2-(tetrahydro-2H-pyran-4-yl)imidazo[1,2-a]pyridine-6-carboxamide;
N-(3-chloro-2-methylphenyl)-7-methoxy-2-(tetrahydro-2H-pyran-4-yl)imidazo[1,2-a]pyridine-6-carboxamide;
N-(2,3-dimethylcyclohexyl)-7-methoxy-2-(tetrahydro-2H-pyran-4-yl)imidazo[1,2-a]pyridine-6-carboxamide;
N-(3-fluoro-5-methylphenyl)-7-methoxy-2-(tetrahydro-2H-pyran-4-yl)imidazo[1,2-a]pyridine-6-carboxamide;
rac-N-((1R,3 S)-3-cyclopropylcyclohexyl)-7-methoxy-2-(tetrahydro-2H-pyran-4-yl)imidazo[1,2-a]pyridine-6-carboxamide;
N-(3,5-dimethylphenyl)-7-methoxy-2-(tetrahydro-2H-pyran-4-yl)imidazo[1,2-a]pyridine-6-carboxamide;
N-(2,3-dihydrobenzofuran-7-yl)-7-methoxy-2-(tetrahydro-2H-pyran-4-yl)imidazo[1,2-a]pyridine-6-carboxamide;
N-(isochroman-5-yl)-7-methoxy-2-(tetrahydro-2H-pyran-4-yl)imidazo[1,2-a]pyridine-6-carboxamide;
N-(3,4-difluoro-2-methylphenyl)-7-methoxy-2-(tetrahydro-2H-pyran-4-yl)imidazo[1,2-a]pyridine-6-carboxamide;
N-(1,6-dimethyl-2-oxo-1,2-dihydropyridin-3-yl)-7-methoxy-2-(tetrahydro-2H-pyran-4-yl)imidazo[1,2-a]pyridine-6-carboxamide;
7-methoxy-N-(1-methyl-2-oxo-1,2-dihydropyridin-3-yl)-2-(tetrahydro-2H-pyran-4-yl)imidazo[1,2-a]pyridine-6-carboxamide;
N-(1-(cyclopropylmethyl)-1H-pyrazol-3-yl)-7-methoxy-2-(tetrahydro-2H-pyran-4-yl)imidazo[1,2-a]pyridine-6-carboxamide;
N-(1-ethyl-2-oxo-1,2-dihydropyridin-3-yl)-7-methoxy-2-(tetrahydro-2H-pyran-4-yl)imidazo[1,2-a]pyridine-6-carboxamide;
N-(5-cyclopropyl-1-methyl-1H-pyrazol-4-yl)-7-methoxy-2-(tetrahydro-2H-pyran-4-yl)imidazo[1,2-a]pyridine-6-carboxamide;
N-(2-isopropylpyridin-3-yl)-7-methoxy-2-(tetrahydro-2H-pyran-4-yl)imidazo[1,2-a]pyridine-6-carboxamide;
N-(6-hydroxy-2-methoxypyridin-3-yl)-7-methoxy-2-(tetrahydro-2H-pyran-4-yl)imidazo[1,2-a]pyridine-6-carboxamide;
N-(2-hydroxypyridin-3-yl)-7-methoxy-2-(tetrahydro-2H-pyran-4-yl)imidazo[1,2-a]pyridine-6-carboxamide;
N-(1-(2-fluoroethyl)-1H-pyrazol-3-yl)-7-methoxy-2-(tetrahydro-2H-pyran-4-yl)imidazo[1,2-a]pyridine-6-carboxamide;
N-(1-cyclopentyl-1H-pyrazol-3-yl)-7-methoxy-2-(tetrahydro-2H-pyran-4-yl)imidazo[1,2-a]pyridine-6-carboxamide;
N-(1-isopropyl-2-oxo-1,2-dihydropyridin-3-yl)-7-methoxy-2-(tetrahydro-2H-pyran-4-yl)imidazo[1,2-a]pyridine-6-carboxamide;
7-methoxy-N-(2-methoxypyridin-3-yl)-2-(tetrahydro-2H-pyran-4-yl)imidazo[1,2-a]pyridine-6-carboxamide;
N-(1,5-dimethyl-1H-pyrazol-4-yl)-7-methoxy-2-(tetrahydro-2H-pyran-4-yl)imidazo[1,2-a]pyridine-6-carboxamide;
7-methoxy-N-(3-methoxypyridin-4-yl)-2-(tetrahydro-2H-pyran-4-yl)imidazo[1,2-a]pyridine-6-carboxamide;
N-(2-(2,2-difluoroethoxy)pyridin-3-yl)-7-methoxy-2-(tetrahydro-2H-pyran-4-yl)imidazo[1,2-a]pyridine-6-carboxamide;
N-(1-(2,2-difluorocyclopropyl)-1H-pyrazol-3-yl)-7-methoxy-2-(tetrahydro-2H-pyran-4-yl)imidazo[1,2-a]pyridine-6-carboxamide;
7-methoxy-N-(2-oxo-1-(2,2,2-trifluoroethyl)-1,2-dihydropyridin-3-yl)-2-(tetrahydro-2H-pyran-4-yl)imidazo[1,2-a]pyridine-6-carboxamide;
N-(5-ethyl-1-methyl-1H-pyrazol-4-yl)-7-methoxy-2-(tetrahydro-2H-pyran-4-yl)imidazo[1,2-a]pyridine-6-carboxamide;
N-(2-isopropoxypyridin-3-yl)-7-methoxy-2-(tetrahydro-2H-pyran-4-yl)imidazo[1,2-a]pyridine-6-carboxamide;
7-methoxy-N-(3-methoxy-2-methylpyridin-4-yl)-2-(tetrahydro-2H-pyran-4-yl)imidazo[1,2-a]pyridine-6-carboxamide;
N-(6-(hydroxymethyl)pyridin-2-yl)-7-methoxy-2-(tetrahydro-2H-pyran-4-yl)imidazo[1,2-a]pyridine-6-carboxamide;
7-methoxy-2-(tetrahydro-2H-pyran-4-yl)-N-(6-(trifluoromethyl)pyridin-2-yl)imidazo[1,2-a]pyridine-6-carboxamide;
7-methoxy-N-(pyrido[3,2-d]pyrimidin-4-yl)-2-(tetrahydro-2H-pyran-4-yl)imidazo[1,2-a]pyridine-6-carboxamide;
N-chroman-8-yl-8-methoxy-2-tetrahydropyran-4-yl-imidazo[1,2-a]pyrazine-6-carboxamide;
N-[6-(difluoromethyl)-2-pyridyl]-8-methoxy-2-tetrahydropyran-4-ylimidazo[1,2-a]pyrazine-6-carboxamide;
N-[6-(difluoromethyl)-2-pyridyl]-8-ethoxy-2-tetrahydropyran-4-ylimidazo[1,2-a]pyrazine-6-carboxamide;
8-methoxy-N-(2-methoxy-3-pyridyl)-2-tetrahydropyran-4-yl-imidazo[1,2-a]pyrazine-6-carboxamide;
8-ethoxy-N-(2-methoxy-3-pyridyl)-2-tetrahydropyran-4-yl-imidazo[1,2-a]pyrazine-6-carboxamide;
N-indan-4-yl-8-methoxy-2-tetrahydropyran-4-yl-imidazo[1,2-a]pyrazine-6-carboxamide; and
N-indan-4-yl-8-ethoxy-2-tetrahydropyran-4-yl-imidazo[1,2-a]pyrazine-6-carboxamide;

or a pharmaceutically acceptable salt thereof.

A forty-second embodiment of the invention provides a pharmaceutical composition comprising a compound according to any one of the preceding embodiments, or a pharmaceutically acceptable salt thereof.

A forty-third embodiment of the invention provides a pharmaceutical composition according to embodiment forty-two, or a pharmaceutically acceptable salt thereof and one or more pharmaceutically acceptable carriers, or diluents.

A forty-fourth embodiment of the invention provides a pharmaceutical composition according to embodiment forty-three, further comprising one or more additional pharmaceutical agent(s).

One embodiment of the invention includes a method of decreasing the expression or activity of IRAK4, or to otherwise affect the properties and/or behavior of IRAK4 polypeptides or polynucleotides comprising administering to said mammal an effective amount of at least one compound described herein, or a pharmaceutically acceptable salt thereof.

A forty-fifth embodiment of the invention is a method of treating an IRAK4 mediated disease in a subject comprising administering to the subject a compound or a pharmaceutically acceptable salt thereof of any one of embodiments one to forty-one or a pharmaceutical composition thereof of any one of embodiments forty-two to forty-four.

A forty-sixth embodiment, the invention provides the use of a compound according to any one of embodiments one to forty-one, for the treatment of a disorder or disease in a subject mediated by IRAK4.

A forty-seventh embodiment, the invention provides the use of a compound according to any one of embodiments one to forty-one in the manufacture of a medicament for the treatment of a disorder or disease in a subject mediated by IRAK4.

A forty-eighth embodiment of the invention comprises a method of treatment according to embodiment forty-five, wherein the IRAK4 mediated disease is selected from an autoimmune disease, an inflammatory disease, bone diseases, metabolic diseases, neurological and neurodegenerative diseases and/or disorders, cancer, cardiovascular diseases, allergies, asthma, Alzheimer's disease, hormone-related diseases, Ischemic stroke, Cerebral Ischemia, hypoxia, TBI (Traumatic Brain Injury), CTE (Chronic Traumatic Encephalopathy), epilepsy, Parkinson's disease (PD), Multiple Sclerosis (MS) and Amyotrophic Lateral Sclerosis (ALS).

A forty-ninth embodiment of the invention comprising a method of treatment according to embodiment forty-five, wherein the IRAK4 mediated disease is selected from disorders and/or conditions associated with inflammation and pain, proliferative diseases, hematopoietic disorders, hematological malignancies, bone disorders, fibrosis diseases and/or disorders, metabolic disorders, muscle diseases and/or disorders, respiratory diseases, pulmonary disorders, genetic development diseases, chronic inflammatory demyelinating neuropathies, vascular or heart diseases, ophthalmic diseases and ocular diseases.

A fiftieth embodiment of the invention comprising a use of a compound according to embodiment forty-seven, wherein the IRAK4 mediated disease is selected from an autoimmune disease, an inflammatory disease, bone diseases, metabolic diseases, neurological and neurodegenerative diseases and/or disorders, cancer, cardiovascular diseases, allergies, asthma, Alzheimer's disease, hormone-related diseases, Ischemic stroke, Cerebral Ischemia, hypoxia, TBI (Traumatic Brain Injury), CTE (Chronic Traumatic Encephalopathy), epilepsy, Parkinson's disease (PD), Multiple Sclerosis (MS) and Amyotrophic Lateral Sclerosis (ALS).

A fifty-first embodiment of the invention comprising a use of a compound according to embodiment forty-seven, wherein the IRAK4 mediated disease is selected from disorders and/or conditions associated with inflammation and pain, proliferative diseases, hematopoietic disorders, hematological malignancies, bone disorders, fibrosis diseases and/or disorders, metabolic disorders, muscle diseases and/or disorders, respiratory diseases, pulmonary disorders, genetic development diseases, chronic inflammatory demyelinating neuropathies, vascular or heart diseases ophthalmic diseases and ocular diseases.

The compounds, or pharmaceutically acceptable salts thereof described herein may be used to decrease the expression or activity of IRAK4, or to otherwise affect the properties and/or behavior of IRAK4 polypeptides or polynucleotides, e.g., stability, phosphorylation, kinase activity, interactions with other proteins, etc.

One embodiment of the invention includes a method of decreasing the expression or activity of IRAK1, or to otherwise affect the properties and/or behavior of IRAK1 polypeptides or polynucleotides comprising administering to said mammal an effective amount of at least one compound described herein, or a pharmaceutically acceptable salt thereof.

In one embodiment, R¹ is elected from the group consisting of

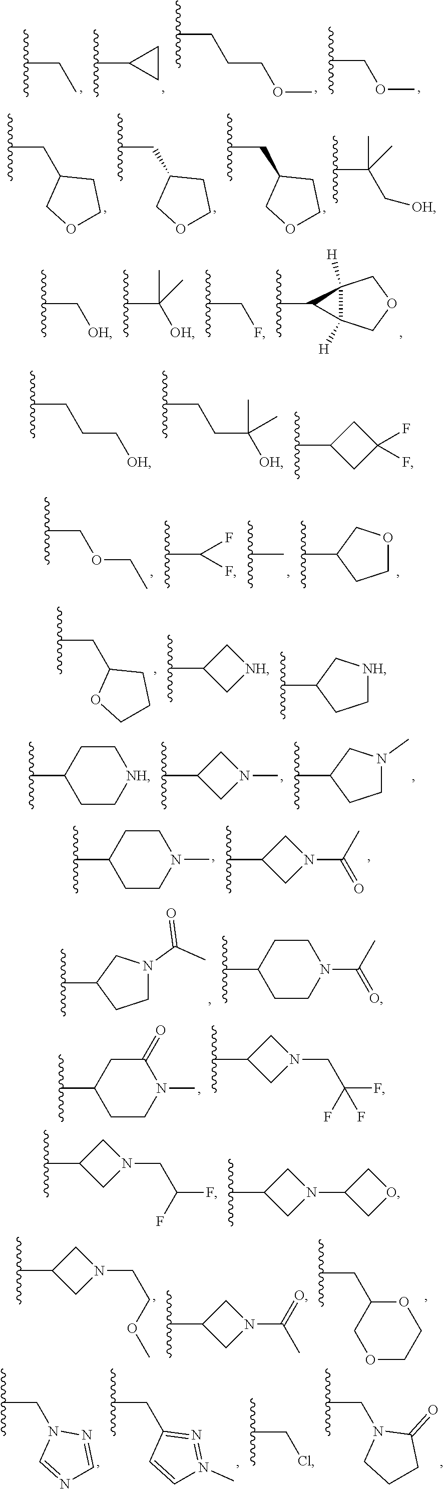

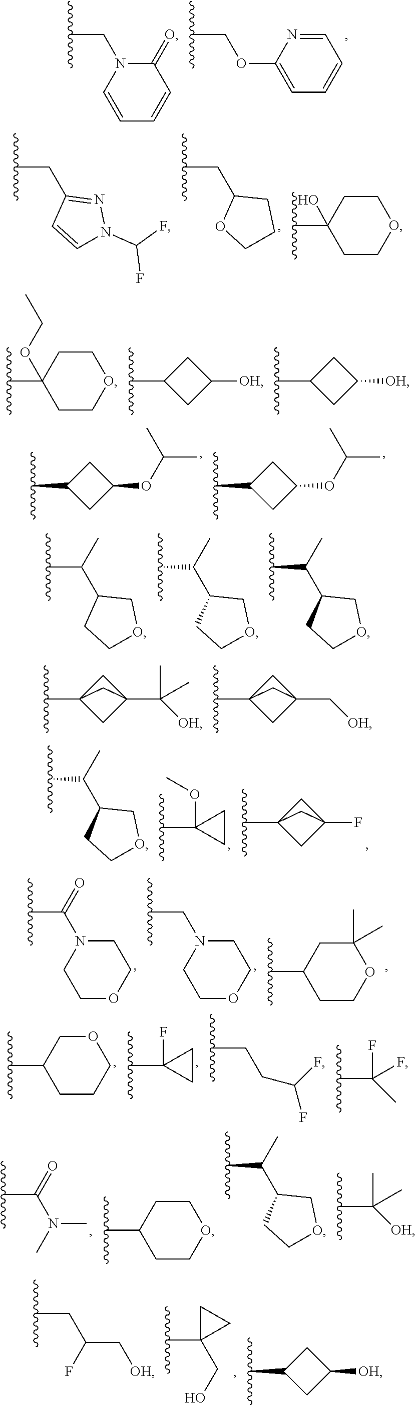

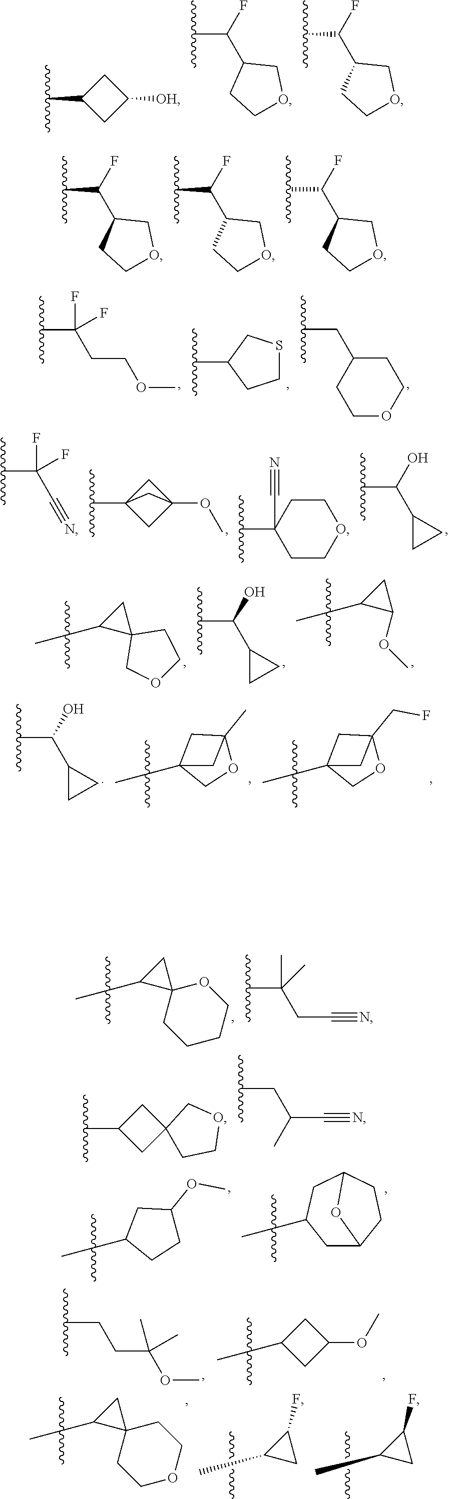

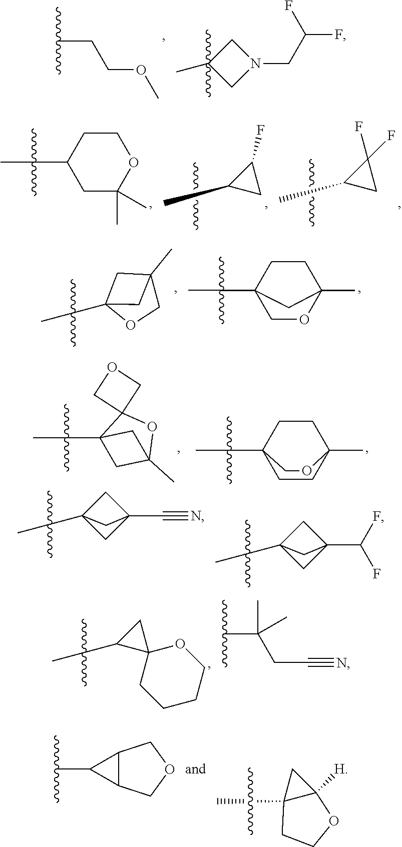

In one embodiment, R¹ is elected from the group consisting of

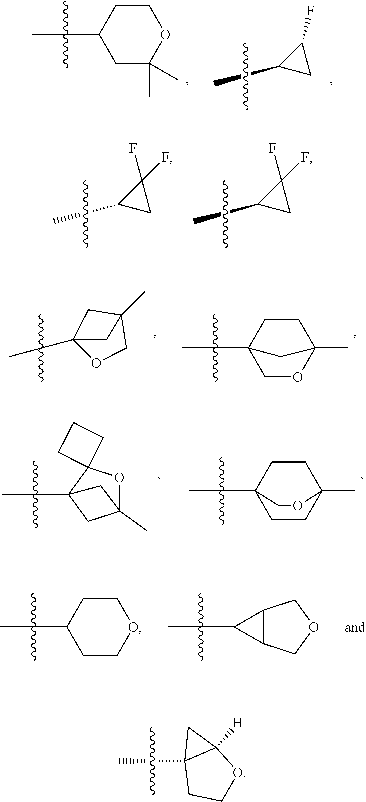

In one embodiment, R³ is elected from the group consisting of

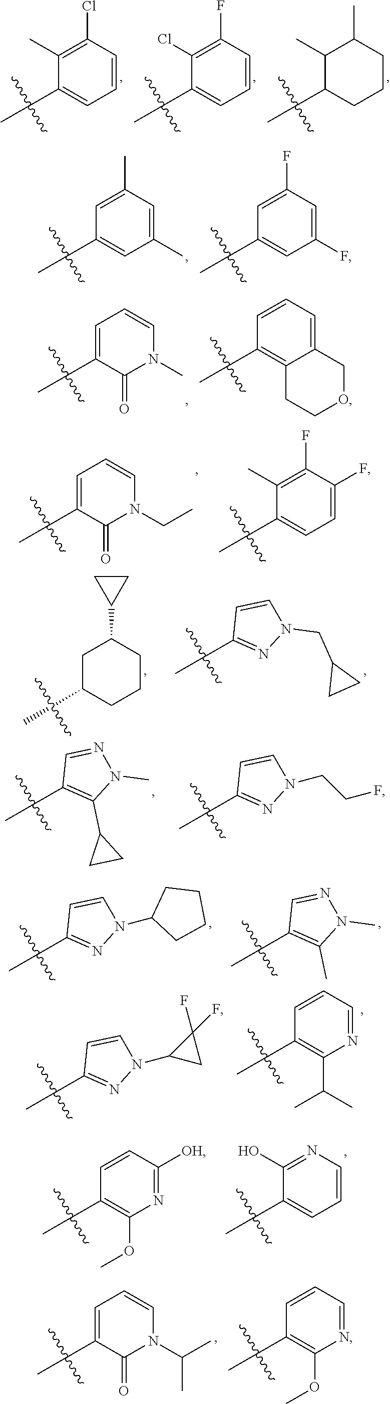

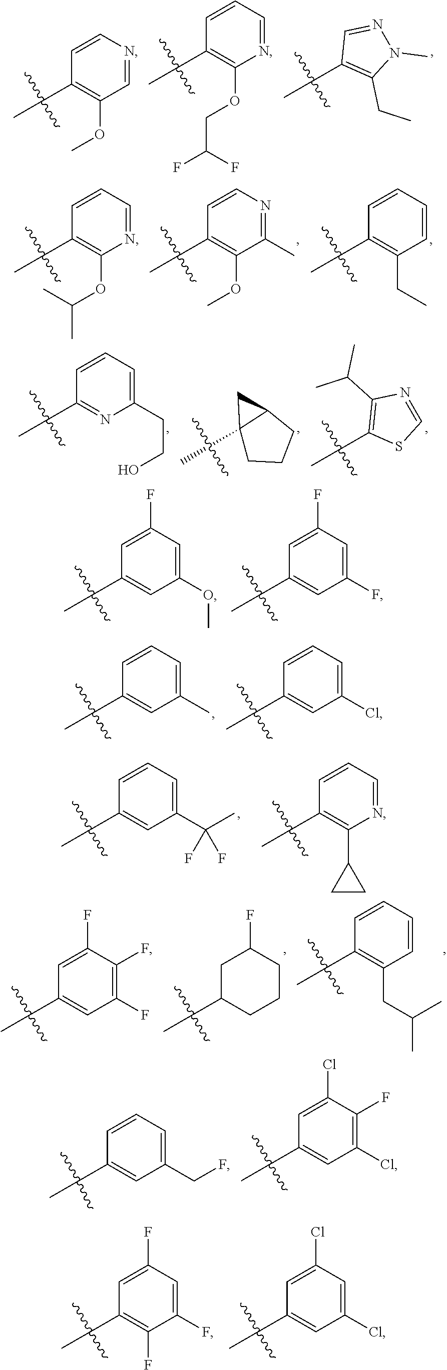

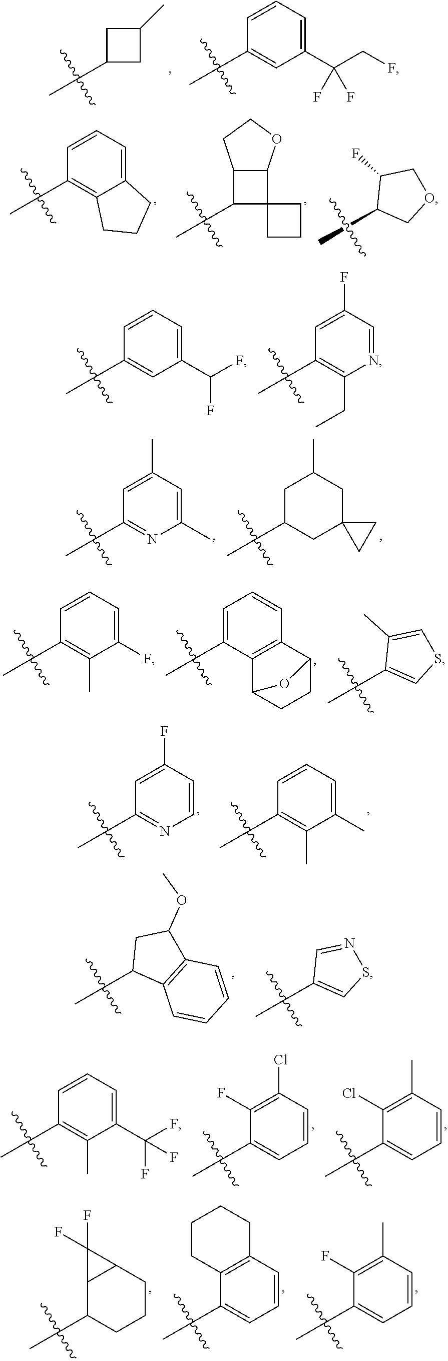

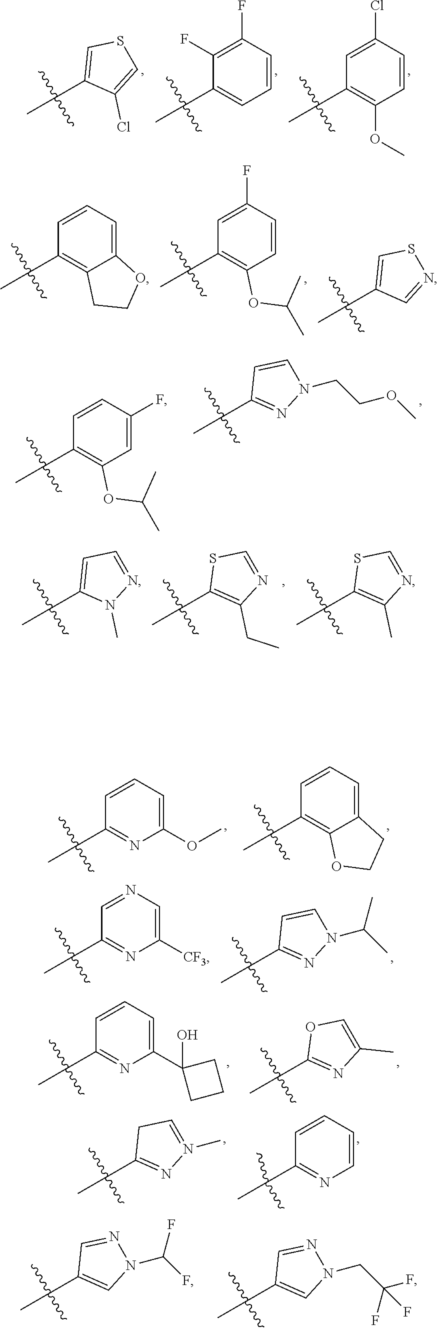

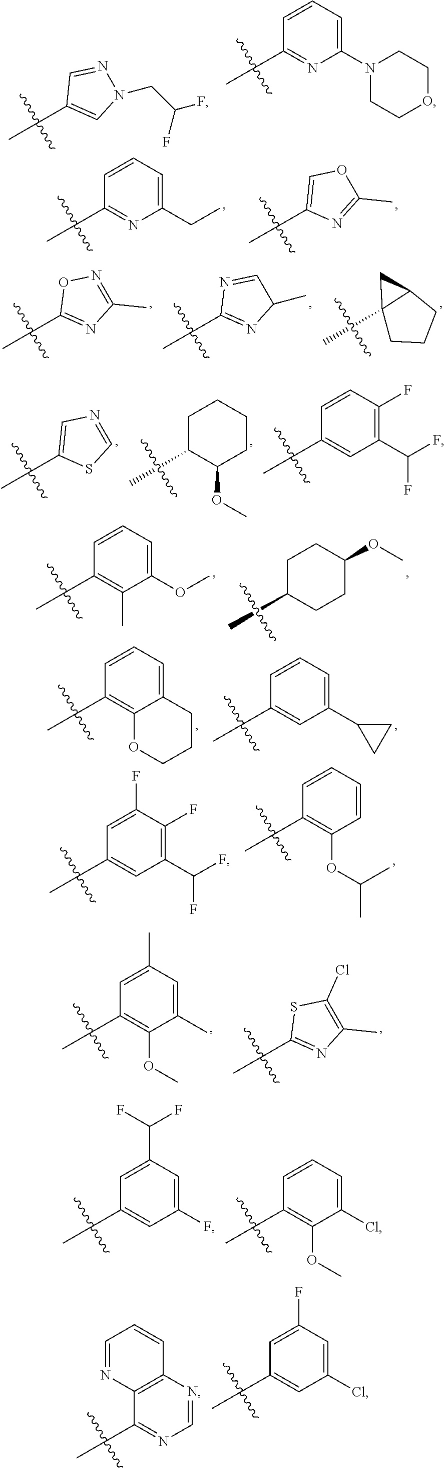

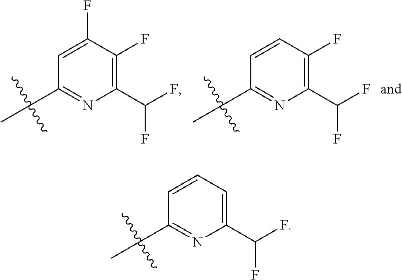 [SMILES: *c1cc(F)c(F)c(C(F)F)n1 ; *c1ccc(F)c(C(F)F)n1 ; "and" ; *c1cccc(C(F)F)n1]

In one embodiment, R³ is elected from the group consisting of

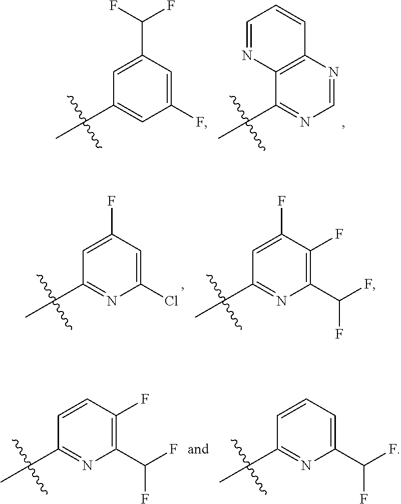 [SMILES: *c1cc(F)cc(C(F)F)c1 ; *c1ncnc2cccnc12 ; *c1cc(F)cc(Cl)n1 ; *c1cc(F)c(F)c(C(F)F)n1 ; *c1ccc(F)c(C(F)F)n1 ; *c1cccc(C(F)F)n1]

In one embodiment, R⁵ is elected from the group consisting of

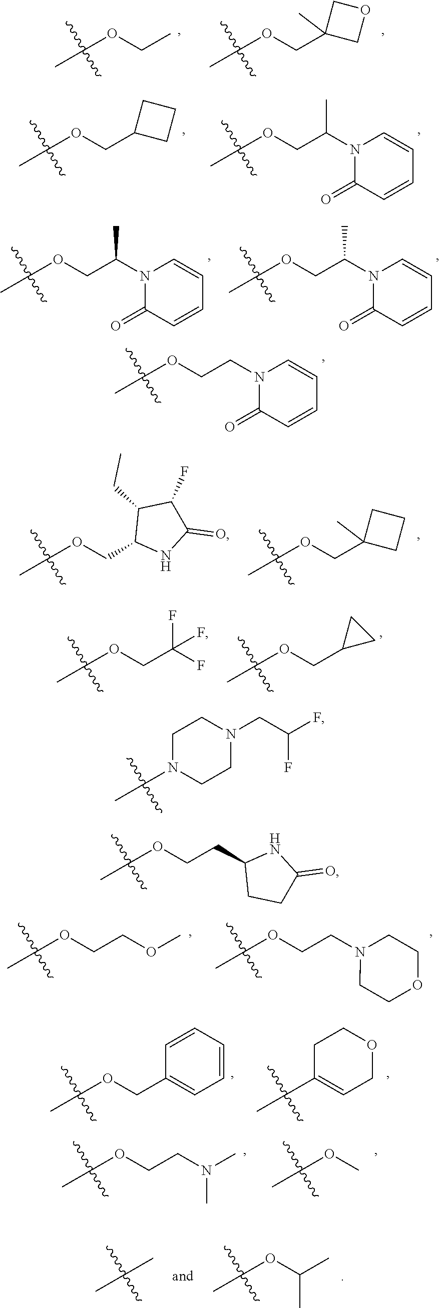

In one embodiment, R⁵ is elected from the group consisting of

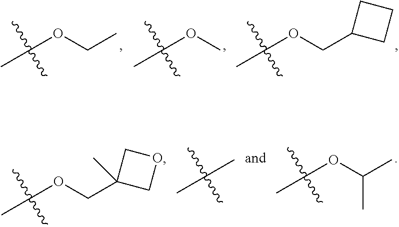

One embodiment of the invention includes a method of decreasing the expression or activity of IRAK4, or to otherwise affect the properties and/or behavior of IRAK4 polypeptides or polynucleotides comprising administering to said subject an effective amount of at least one compound described herein, or a pharmaceutically acceptable salt thereof.

One embodiment of the invention includes a method for treating an inflammatory disease in a subject, the method comprising administering to the patient a therapeutically effective amount of a compound described herein, or a pharmaceutically acceptable salt thereof, thereby treating the inflammatory disease in the subject.

In one embodiment, the inflammatory disease is a pulmonary disease or a disease of the airway.

In one embodiment, the pulmonary disease and disease of the airway is selected from Adult Respiratory Disease Syndrome (ARDS), Chronic Obstructive Pulmonary Disease (COPD), pulmonary fibrosis, interstitial lung disease, asthma, chronic cough, and allergic rhinitis.

In one embodiment, the inflammatory disease is selected from transplant rejection, CD14 mediated sepsis, non-CD14 mediated sepsis, inflammatory bowel disease, Behcet's syndrome, ankylosing spondylitis, sarcoidosis, and gout.

One embodiment of the invention includes a method for treating an autoimmune disease, cancer, cardiovascular disease, a disease of the central nervous system, a disease of the skin, an ophthalmic disease and condition, and bone disease in a subject, the method comprising administering to the patient a therapeutically effective amount of a compound disclosed herein, or a pharmaceutically acceptable salt thereof, thereby treating the autoimmune disease, cancer, cardiovascular disease, disease of the central nervous system, disease of the skin, ophthalmic disease and condition, and bone disease in the subject.

In one embodiment, the autoimmune disease is selected from rheumatoid arthritis, systemic lupus erythematosus, multiple sclerosis, diabetes, systemic sclerosis, and Sjogren's syndrome.

In one embodiment, the autoimmune disease is type 1 diabetes.

In one embodiment, the cancer is selected from Waldenstrim's macroglobulinemia, solid tumors, skin cancer, and lymphoma.

In one embodiment, the cardiovascular disease is selected from stroke and atherosclerosis.

In one embodiment, the disease of the central nervous system is a neurodegenerative disease.

In one embodiment, the disease of the skin is selected from rash, contact dermatitis, psoriasis, and atopic dermatitis.

In one embodiment, the bone disease is selected from osteoporosis and osteoarthritis.

In one embodiment, the inflammatory bowel disease is selected from Crohn's disease and ulcerative colitis.

One embodiment of the invention includes a method for treating an ischemic fibrotic disease, the method comprising administering to the patient a therapeutically effective amount of a compound described herein, or a pharmaceutically acceptable salt thereof, thereby treating the ischemic fibrotic disease in the subject. In one embodiment, the ischemic fibrotic disease is selected from stroke, acute lung injury, acute kidney injury, ischemic cardiac injury, acute liver injury, and ischemic skeletal muscle injury.

One embodiment of the invention includes a method for treating post-organ transplantation fibrosis, the method comprising administering to the patient a therapeutically effective amount of a compound described herein, or a pharmaceutically acceptable salt thereof, thereby treating post-organ transplantation fibrosis in the subject.

One embodiment of the invention includes a method for treating hypertensive or diabetic end organ disease, the method comprising administering to the patient a therapeutically effective amount of a compound described herein, or a pharmaceutically acceptable salt thereof, thereby treating hypertensive or diabetic end organ disease in the subject.

One embodiment of the invention includes a method for treating hypertensive kidney disease, the method comprising administering to the patient a therapeutically effective amount of a compound described herein, or a pharmaceutically acceptable salt thereof, thereby treating hypertensive kidney disease in the subject.

One embodiment of the invention includes a method for treating idiopathic pulmonary fibrosis (IPF), the method comprising administering to the patient a therapeutically effective amount of a compound described herein, or a pharmaceutically acceptable salt thereof, thereby treating IPF in the subject.

One embodiment of the invention includes a method for treating scleroderma or systemic sclerosis, the method comprising administering to the patient a therapeutically effective amount of a compound described herein, or a pharmaceutically acceptable salt thereof, thereby treating scleroderma or systemic sclerosis in the subject.

One embodiment of the invention includes a method for treating liver cirrhosis, the method comprising administering to the patient a therapeutically effective amount of a compound described herein, or a pharmaceutically acceptable salt thereof, thereby treating liver cirrhosis in the subject.

One embodiment of the invention includes a method for treating fibrotic diseases wherein tissue injury and/or inflammation are present, the method comprising administering to the patient a therapeutically effective amount of a compound described herein, or a pharmaceutically acceptable salt thereof, thereby treating fibrotic diseases where tissue injury and/or inflammation are present in the subject. The fibrotic diseases include, for example, pancreatitis, peritonitis, burns, glomerulonephritis, complications of drug toxicity, and scarring following infections.

Scarring of the internal organs is a major global health problem, which is the consequence of subclinical injury to the organ over a period of time or as the sequela of acute severe injury or inflammation. All organs may be affected by scarring and currently there are few therapies the specifically target the evolution of scarring. Increasing evidence indicates that scarring per se provokes further decline in organ function, inflammation and tissue ischemia. This may be directly due the deposition of the fibrotic matrix which impairs function such as in contractility and relaxation of the heart and vasculature or impaired inflation and deflation of lungs, or by increasing the space between microvasculature and vital cells of the organ that are deprived of nutrients and distorting normal tissue architecture. However recent studies have shown that myofibroblasts themselves are inflammatory cells, generating cytokines, chemokines and radicals that promote injury; and myofibroblasts appear as a result of a transition from cells that normally nurse and maintain the microvasculature, known as pericytes. The consequence of this transition of phenotype is an unstable microvasculature that leads to aberrant angiogenesis, or rarefaction.

The present disclosure relates to methods and compositions for treating, preventing, and/or reducing scarring in organs. More particularly, the present disclosure relates to methods and composition for treating, preventing, and/or reducing scarring in kidneys.

It is contemplated that the present disclosure, methods and compositions described herein can be used as an antifibrotic, or used to treat, prevent, and/or reduce the severity and damage from fibrosis.

It is additionally contemplated that the present disclosure, methods and compositions described herein can be used to treat, prevent, and/or reduce the severity and damage from fibrosis.

It is further contemplated that the present disclosure, methods and compositions described herein can used as an anti-inflammatory, used to treat inflammation.

Some non-limiting examples of organs include: kidney, hearts, lungs, stomach, liver, pancreas, hypothalamus, stomach, uterus, bladder, diaphragm, pancreas, intestines, colon, and so forth.

In certain embodiments, the present invention relates to the aforementioned methods, wherein said compound is administered parenterally.

In certain embodiments, the present invention relates to the aforementioned methods, wherein said compound is administered intramuscularly, intravenously, subcutaneously, orally, pulmonary, rectally, intrathecally, topically or intranasally.

In certain embodiments, the present invention relates to the aforementioned methods, wherein said compound is administered systemically.

In certain embodiments, the present invention relates to the aforementioned methods, wherein said subject is a mammal.

In certain embodiments, the present invention relates to the aforementioned methods, wherein said subject is a primate.

In certain embodiments, the present invention relates to the aforementioned methods, wherein said subject is a human.

The compounds and intermediates described herein may be isolated and used as the compound per se. Alternatively, when a moiety is present that is capable of forming a salt, the compound or intermediate may be isolated and used as its corresponding salt. As used herein, the terms “salt” or “salts” refers to an acid addition or base addition salt of a compound of the invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The salts can be synthesized by conventional chemical methods from a compound containing a basic or acidic moiety. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

It will be recognized by those skilled in the art that the compounds of the present invention may contain chiral centers and as such may exist in different stereoisomeric forms. As used herein, the term “an optical isomer” or “a stereoisomer” refers to any of the various stereo isomeric configurations which may exist for a given compound of the present invention. It is understood that a substituent may be attached at a chiral center of a carbon atom. Therefore, the invention includes enantiomers, diastereomers or racemates of the compound.

“Enantiomers” are a pair of stereoisomers that are non-superimposable mirror images of each other. A 1:1 mixture of a pair of enantiomers is a “racemic” mixture. The term is used to designate a racemic mixture where appropriate. When designating the stereochemistry for the compounds of the present invention, a single stereoisomer with known relative and absolute configuration of the two chiral centers is designated using the conventional RS system (e.g., (1S,2S)); a single stereoisomer with known relative configuration but unknown absolute configuration is designated with stars (e.g., (1R*,2R*)); and a racemate with two letters (e.g, (1RS,2RS) as a racemic mixture of (1R,2R) and (1S,2S); (1RS,2SR) as a racemic mixture of (1R,2S) and (1S,2R)). “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)- or (S)—.

Unless specified otherwise, the compounds of the present invention are meant to include all such possible stereoisomers, including racemic mixtures, optically pure forms and intermediate mixtures. Optically active (R)- and (S)-stereoisomers may be prepared using chiral synthons or chiral reagents, or resolved using conventional techniques (e.g., separated on chiral SFC or HPLC chromatography columns, such as CHIRALPAK^{RTM} and CHIRALCEL^{RTM} available from DAICEL Corp. using the appropriate solvent or mixture of solvents to achieve good separation). If the compound contains a double bond, the substituent may be E or Z configuration. If the compound contains a disubstituted cycloalkyl, the cycloalkyl substituent may have a cis- or trans-configuration. All tautomeric forms are also intended to be included.

### Pharmacology and Utility

Compounds of the present invention have been found to modulate IRAK4 activity and may be beneficial for the treatment of neurological, neurodegenerative and other additional diseases

Another aspect of the invention provides a method for treating or lessening the severity of a disease, disorder, or condition associated with the modulation of IRAK4 in a subject, which comprises administering to the subject a compound of Formula (I′) or (I) or a pharmaceutically acceptable salt thereof.

In certain embodiments, the present invention provides a method of treating a condition, disease or disorder implicated by a deficiency of IRAK4 activity, the method comprising administering a composition comprising a compound of formula (I′) or (I) to a subject, preferably a mammal, in need of treatment thereof.

According to the invention an “effective dose” or an “effective amount” of the compound or pharmaceutical composition is that amount effective for treating or lessening the severity of one or more of the diseases, disorders or conditions as recited above.

The compounds and compositions, according to the methods of the present invention, may be administered using any amount and any route of administration effective for treating or lessening the severity of one or more of the diseases, disorders or conditions recited above.

The compounds of the present invention are typically used as a pharmaceutical composition (e.g., a compound of the present invention and at least one pharmaceutically acceptable carrier). As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. For purposes of this invention, solvates and hydrates are considered pharmaceutical compositions comprising a compound of the present invention and a solvent (i.e., solvate) or water (i.e., hydrate).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present invention is generally formulated for use as a parenteral or oral administration or alternatively suppositories.

For example, the pharmaceutical oral compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with

a) diluents, e.g., lactose, dextrose, sucrose, mannitol, sorbitol, cellulose and/or glycine;

b) lubricants, e.g., silica, talcum, stearic acid, its magnesium or calcium salt and/or polyethylene glycol; for tablets also

c) binders, e.g., magnesium aluminum silicate, starch paste, gelatin, tragacanth, methylcellulose, sodium carboxymethylcellulose and/or polyvinylpyrrolidone; if desired

d) disintegrants, e.g., starches, agar, alginic acid or its sodium salt, or effervescent mixtures; and/or

e) absorbents, colorants, flavors and sweeteners.

Tablets may be either film coated or enteric coated according to methods known in the art.

Suitable compositions for oral administration include a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compound of the present invention or pharmaceutical composition thereof for use in a subject (e.g., human) is typically administered orally or parenterally at a therapeutic dose of less than or equal to about 100 mg/kg, 75 mg/kg, 50 mg/kg, 25 mg/kg, 10 mg/kg, 7.5 mg/kg, 5.0 mg/kg, 3.0 mg/kg, 1.0 mg/kg, 0.5 mg/kg, 0.05 mg/kg or 0.01 mg/kg, but preferably not less than about 0.0001 mg/kg. When administered intravenously via infusion, the dosage may depend upon the infusion rate at which an IV formulation is administered. In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations.

### Combination Therapy

The compounds of the present invention can be used, alone or in combination with other therapeutic agents, in the treatment of various conditions or disease states. The compound(s) of the present invention and other therapeutic agent(s) may be administered simultaneously (either in the same dosage form or in separate dosage forms) or sequentially.

Two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

The present invention includes the use of a combination of an IRAK inhibitor compound as provided in the compound of formula (I) and one or more additional pharmaceutically active agent(s). If a combination of active agents is administered, then they may be administered sequentially or simultaneously, in separate dosage forms or combined in a single dosage form. Accordingly, the present invention also includes pharmaceutical compositions comprising an amount of: (a) a first agent comprising a compound of formula (I) or a pharmaceutically acceptable salt of the compound; (b) a second pharmaceutically active agent; and (c) a pharmaceutically acceptable carrier, vehicle or diluent.

The compounds of the present invention can be administered alone or in combination with one or more additional therapeutic agents. By “administered in combination” or “combination therapy” it is meant that a compound of the present invention and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect. Thus, the methods of prevention and treatment described herein include use of combination agents.

The combination agents are administered to a mammal, including a human, in a therapeutically effective amount. By “therapeutically effective amount” it is meant an amount of a compound of the present invention that, when administered alone or in combination with an additional therapeutic agent to a mammal, is effective to treat the desired disease/condition e.g., inflammatory condition such as systemic lupus erythematosus. See also, T. Koutsokeras and T. Healy, Systemic lupus erythematosus and lupus nephritis, Nat Rev Drug Discov, 2014, 13(3), 173-174, for therapeutic agents useful treating lupus.

In particular, it is contemplated that the compounds of the invention may be administered with the following therapeutic agents: Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as Tecfidera® and beta interferon (e.g., Avonex® and Rebif®), Copaxone®, and mitoxantrone; treatments for asthma such as albuterol and Singulair®; agents for treating schizophrenia such as zyprexa, risperdal seroquel, and haloperidol; anti-inflammatory agents such as corticosteroids. T F blockers IL-1 RA, azathioprine, cyclophosphamide; and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3 A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

### Definitions

As used herein, a “patient,” “subject” or “individual” are used interchangeably and refer to either a human or non-human animal. The term includes mammals such as humans. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. Preferably, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder, refers to the management and care of a patient for the purpose of combating the disease, condition, or disorder and includes the administration of a compound of the present invention to prevent the onset of the symptoms or complications, alleviating the symptoms or complications, or eliminating the disease, condition or disorder.

As used herein the term “stroke” has the meaning normally accepted in the art. The term can broadly refer to the development of neurological deficits associated with the impaired blood flow regardless of cause. Potential causes include, but are not limited to, thrombosis, hemorrhage and embolism. The term “ischemic stroke” refers more specifically to a type of stroke that is of limited extent and caused due to a blockage of blood flow.

As used herein, a subject is “in need of” a treatment if such subject would benefit biologically, medically or in quality of life from such treatment (preferably, a human).

As used herein the term “co-administer” refers to the presence of two active agents in the blood of an individual. Active agents that are co-administered can be concurrently or sequentially delivered.

The term “combination therapy” or “in combination with” or “pharmaceutical combination” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent being administered prior to, concurrent with, or sequentially to each other with no specific time limits. In each case, the treatment regimen will provide beneficial effects of the drug combination in treating the conditions or disorders described herein.

As used herein, the phrase “optionally substituted” is used interchangeably with the phrase “substituted or unsubstituted.” In general the term “optionally substituted” refers to the replacement of hydrogen radicals in a given structure with the radical of a specified substituent. Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position.

As used herein, the term “C₁₋₅alkyl” refers to a fully saturated branched or unbranched hydrocarbon moiety having 1 to 5 carbon atoms. The terms “C₁₋₄alkyl”, “C₁₋₃alkyl” and “C₁₋₂alkyl” are to be construed accordingly. Representative examples of “C₁₋₅alkyl” include, but are not limited to, methyl, ethyl, n-propyl, iso-propyl, n-butyl, sec-butyl, iso-butyl, tert-butyl, n-pentyl, isopentyl and neopentyl. Similarly, the alkyl portion (i.e., alkyl moiety) of an alkoxy have the same definition as above. When indicated as being “optionally substituted”, the alkane radical or alkyl moiety may be unsubstituted or substituted with one or more substituents (generally, one to three substituents except in the case of halogen substituents such as perchloro or perfluoroalkyls). “Halo-substituted alkyl” refers to an alkyl group having at least one halogen substitution.

As used herein, the term “C₁₋₄ alkoxy” refers to a fully saturated branched or unbranched alkyl moiety attached through an oxygen bridge (i.e. a —O— C₁₋₄ alkyl group wherein C₁₋₄ alkyl is as defined herein). Representative examples of alkoxy include, but are not limited to, methoxy, ethoxy, propoxy, 2-propoxy, butoxy, tert-butoxy and the like. Preferably, alkoxy groups have about 1-4 carbons, more preferably about 1-2 carbons. The term “C₁₋₂ alkoxy” is to be construed accordingly.

As used herein, the term “C₁₋₄ alkoxy-C₁₋₄ alkyl” refers to a C₁₋₄ allkyl group as defined herein, wherein at least of the hydrogen atoms is replaced by an C₁₋₄ alkoxy. The C₁₋₄ alkoxy-C₁₋₄ alkyl group is connected through the rest of the molecule described herein through the alkyl group.

“Halogen” or “halo” may be fluorine, chlorine, bromine or iodine (preferred halogens as substituents are fluorine and chlorine).

As used herein, the term “halo-substituted-C₁₋₄alkyl” or “halo-C₁₋₄ alkyl” refers to a C₁₋₄ alkyl group as defined herein, wherein at least one of the hydrogen atoms is replaced by a halo atom. The halo-C₁₋₄alkyl group can be monohalo-C₁₋₄alkyl, dihalo-C₁₋₄alkyl or polyhalo-C₁₋₄ alkyl including perhalo-C₁₋₄alkyl. A monohalo-C₁₋₄alkyl can have one iodo, bromo, chloro or fluoro within the alkyl group. Dihalo-C₁₋₄alkyl and polyhalo-C₁₋₄alkyl groups can have two or more of the same halo atoms or a combination of different halo groups within the alkyl. Typically the polyhalo-C₁₋₄alkyl group contains up to 9, or 8, or 7, or 6, or 5, or 4, or 3, or 2 halo groups. Non-limiting examples of halo-C₁₋₄alkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-C₁₋₄alkyl group refers to a C₁₋₄alkyl group having all hydrogen atoms replaced with halo atoms.

As used herein, the term “halo-substituted-C₁₋₄alkoxy” or “halo-C₁₋₄alkoxy” refers to C₁₋₄ alkoxy group as defined herein above wherein at least one of the hydrogen atoms is replaced by a halo atom. Non-limiting examples of halo-substituted-C₁₋₄alkoxy include fluoromethoxy, difluoromethoxy, trifluoromethoxy, chloromethoxy, dichloromethoxy, trichloromethoxy, difluorochloromethoxy, dichlorofluoromethoxy, difluoroethoxy, difluoropropoxy, dichloroethoxy and dichloropropoxy and the like.

As used herein “Hydroxyl” or “Hydroxy” refers to the group —OH.

As used herein, the term “hydroxy-substituted-C₁₋₄ alkyl” refers to a C₁₋₄ alkyl group as defined herein, wherein at least one of the hydrogen atoms is replaced by a hydroxyl group. The hydroxy-substituted-C₁₋₄ alkyl group can be monohydroxy-C₁₋₄ alkyl, dihydroxy-C₁₋₄ alkyl or polyhydroxy-C₁₋₄ alkyl including perhydroxy-C₁₋₄ alkyl. A monohydroxy-C₁₋₄ alkyl can have one hydroxyl group within the alkyl group. Dihydroxy-C₁₋₄ alkyl and polyhydroxy-C₁₋₄ alkyl groups can have two or more of the same hydroxyl groups or a combination of different hydroxyl groups within the alkyl. Typically the polyhydroxy-C₁₋₄alkyl group contains up to 9, or 8, or 7, or 6, or 5, or 4, or 3, or 2 hydroxy groups. Non-limiting examples of hydroxy substituted-C₁₋₄ alkyl include hydroxy-methyl, dihydroxy-methyl, pentahydroxy-ethyl, dihydroxyethyl, and dihydroxypropyl. A perhydroxy-C₁₋₄ alkyl group refers to a C₁₋₄ alkyl group having all hydrogen atoms replaced with hydroxy atoms.

The term “oxo” (═O) refers to an oxygen atom connected to a carbon or sulfur atom by a double bond. Examples include carbonyl, sulfinyl, or sulfonyl groups (—C(O)—, —S(O)— or —S(O)₂—) such as, a ketone, aldehyde, or part of an acid, ester, amide, lactone, or lactam group and the like.

The term “aryl or C₆₋₁₀aryl” refers to 6- to 10-membered aromatic carbocyclic moieties having a single (e.g., phenyl) or a fused ring system (e.g., naphthalene). A typical aryl group is phenyl group.

The term “fully or partially saturated carbocyclic ring” refers to a nonaromatic hydrocarbon ring that is either partially or fully saturated and may exist as a single ring, bicyclic ring (including fused, spiral or bridged carbocyclic rings) or a spiral ring. Unless specified otherwise, the carbocyclic ring generally contains 4- to 7-ring members.

The term “C₃₋₆ cycloalkyl” refers to a carbocyclic ring which is fully saturated (e.g., cyclopropyl, cyclobutyl, cyclopentyl, and cyclohexyl).

The term “fully or partially saturated C₃₋₆ cycloalkyl” refers to a carbocyclic ring which is fully saturated (e.g., cyclopropyl, cyclobutyl, cyclopentyl, and cyclohexyl) or partially saturated (e.g. cyclopropenyl, cyclobutenyl, cyclopentenyl, and cyclohexenyl).

The term “4 to 7 membered heterocycle” or “C₄₋₇ heterocycle” refers to a monocyclic ring which is fully saturated which has 4 to 7 ring atoms which contains 1 to 2 heteroatoms, independently selected from sulfur, oxygen and/or nitrogen. A typical “C₄₋₇ heterocycle” group includes oxtanyl, tetrahydrofuranyl, dihydrofuranyl, 1,4-dioxanyl, morpholinyl, 1,4-dithianyl, piperazinyl, piperidinyl, 1,3-dioxolanyl, pyrrolinyl, pyrrolidinyl, tetrahydropyranyl, oxathiolanyl, dithiolanyl, 1,3-dioxanyl, 1,3-dithianyl, oxathianyl, thiomorpholinyl, thiomorpholinyl 1,1 dioxide, tetrahydro-thiopyran 1,1-dioxide, 1,4-diazepanyl. In some embodiments, a “C₄₋₇ heterocycle” group contains at least one oxygen ring atom. In some embodiments, a “C₄₋₇ heterocycle” group is selected from oxtanyl, tetrahydrofuranyl, 1,4-dioxanyl and tetrahydropyranyl.

The term “fully or partially saturated heterocycle” or “fully or partially saturated 4 to 7 membered heterocycle” refers to a nonaromatic ring that is either partially or fully saturated and may exist as a single ring, bicyclic ring (including fused heterocyclic rings) or a spiral ring. Unless specified otherwise, the heterocyclic ring is generally a 4 to 7-membered ring containing 1 to 3 heteroatoms (preferably 1, 2 or 3 heteroatoms) independently selected from sulfur, oxygen and/or nitrogen. A partially saturated heterocyclic ring also includes groups wherein the heterocyclic ring is fused to an aryl or heteroaryl ring (e.g., 2,3-dihydrobenzofuranyl, indolinyl (or 2,3-dihydroindolyl), 2,3-dihydrobenzothiophenyl, 2,3-dihydrobenzothiazolyl, 1,2,3,4-tetrahydroquinolinyl, 1,2,3,4-tetrahydroisoquinolinyl, 5,6,7,8-tetrahydropyrido[3,4-b]pyrazinyl).

As used herein the term “spiral” or “spiro 5 to 10 membered heterobicyclic ring system” means a two-ring system wherein both rings share one common atom. Examples of spiral rings include oxaspiro[2.4]heptanyl, 5-oxaspiro[2.4]heptanyl, 4-oxaspiro[2.4]heptane, 4-oxaspiro[2.5]octanyl, 6-oxaspiro[2.5]octanyl, oxaspiro[2.5]octanyl, oxaspiro[3.4]octanyl, oxaspiro[bicyclo[2.1.1]hexane-2,3′-oxetan]-1-yl, oxaspiro[bicyclo[3.2.0]heptane-6,1′-cyclobutan]-7-yl, 2,6-diazaspiro[3.3]heptanyl, -oxa-6-azaspiro[3.3]heptane, 2,2,6-diazaspiro[3.3]heptane, 3-azaspiro[5.5]undecanyl, 3,9-diazaspiro[5.5]undecanyl, 7-azaspiro[3.5]nonane, 2,6-diazaspiro[3.4]octane, 8-azaspiro[4.5]decane, 1,6-diazaspiro[3.3]heptane, 5-azaspiro[2.5]octane, 4,7-diazaspiro[2.5]octane, 5-oxa-2-azaspiro[3.4]octane, 6-oxa-1-azaspiro[3.3]heptane, 3-azaspiro[5.5]undecanyl, 3,9-diazaspiro[5.5]undecanyl, and the like.

As used herein the term “spiro 3-8 membered cycloalkyl” means a two-ring system wherein both rings share one common carbon atom. Examples of spiro 3-8 membered cycloalkyl rings include spiro[2.5]octane, spiro[2.3]hexane, spiro[2.4]heptane, spiro[3.4]octane and the like.

Partially saturated or fully saturated heterocyclic rings include groups such as epoxy, aziridinyl, azetidinyl, tetrahydrofuranyl, dihydrofuranyl, dihydropyridinyl, pyrrolidinyl, imidazolidinyl, imidazolinyl, 1H-dihydroimidazolyl, hexahydropyrimidinyl, piperidinyl, piperazinyl, pyrazolidinyl, 2H-pyranyl, 4H-pyranyl, oxazinyl, morpholino, thiomorpholino, tetrahydrothienyl, tetrahydrothienyl 1,1-dioxide, oxazolidinyl, thiazolidinyl, 7-oxabicyclo[2.2.1]heptane, and the like.

The term “Fused heterocycle” or “7 to 10 membered fused heterobicyclic ring system” or “5 to 10 membered fused heterobicyclic ring system” refers to two ring systems share two adjacent ring atoms ad at least one the ring systems contain a ring atom that is a heteroatom selected from O, N and S. Examples of fused heterocycles include fully or partially saturated groups and bicyclic heteroaryls, such as 1,3-dihydroisobenzofuran, 4-methyl-3,4-dihydro-2H-benzo[b][1,4]oxazine, pyrazolo[1,5-a]pyrimidine, 2-oxabicyclo[2.1.0]pentane, 5,6-dihydro-4H-pyrrolo[1,2-b]pyrazole, 6,7-dihydro-5H-cyclopenta[b]pyridine, indolin-2-one, 2,3-dihydrobenzofuran, 1-methyl-2-oxo-1,2,3,4-tetrahydroquinoline, 3,4-dihydroquinolin-2(1H)-one, chromane and isochromane, 4,5,6,7-tetrahydro-3H-imidazo[4,5-c]pyridine, 8-azabicyclo[3.2.1]octan-3-ol, octahydropyrrolo[1,2-a]pyrazine, 5,6,7,8-tetrahydroimidazo[1,2-a]pyrazine, 3,8 diazabicyclo[3.2.1]octane, 8-oxa-3-azabicyclo[3.2.1]octane, 7-oxabicyclo[2.2.1]heptane, 1H-pyrazole, 2,5-diazabicyclo[2.2.1]heptane, 5,6,7,8-tetrahydro-[1,2,4]triazolo[4,3-a]pyrazine, 3-oxabicyclo[3.1.0]hexane, or 3-azabicyclo[3.1.0]hexane. A partially saturated heterocyclic ring also includes groups wherein the heterocyclic ring is fused to an aryl or heteroaryl ring (e.g., 2,3-dihydrobenzofuranyl, indolinyl (or 2,3-dihydroindolyl), 2,3-dihydrobenzothiophenyl, 2,3-dihydrobenzothiazolyl, 1,2,3,4-tetrahydroquinolinyl, 1,2,3,4-tetrahydroisoquinolinyl, 5,6,7,8-tetrahydropyrido[3,4-b]pyrazinyl, 6,7-dihydro-5H-pyrazolo[5,1-b][1,3]oxazine, and the like). In some embodiments, the “7 to 10 membered fused heterobicyclic ring system” is a 9 to 10 membered bicyclic heteroaryl, such as pyrazolo[1,5-a]pyrimidine, pyrazolo[1,5-a]pyridine, [1,2,4]triazolo[4,3-a]pyridine, [1,2,4]triazolo[1,5-a]pyridine, isothiazolo[4,3-b]pyridine, pyrrolo[1,2-a]pyrimidine, pyrido[3,2-d]pyrimidine, imidazo[1,2-b]pyridazine, thieno[2,3-b]pyrazine, 1H-benzo[d]imidazole, benzo[d]thiazole, 1,6-naphthyridine and 1,5-naphthyridine.

As used herein the term “7 to 10 membered fused bicyclic ring system” refers to a 7 to 10 membered carbocyclic moiety connected at two non-adjacent ring atoms of the carbocycle (e.g. 1,2,3,4-tetrahydronaphthalene, (1S,5R)-1-methylbicyclo[3.1.0]hexane, bicyclo[3.1.0]hexane, bicyclo[4.1.0]heptane and 2,3-dihydro-1H-indene.

As used herein the term “bridged-carbocyclic ring” refers to a 5 to 10 membered cyclic moiety connected at two non-adjacent ring atoms of the carbocycle (e.g. bicyclo[1.1.1]pentane, bicyclo[2.2.1]heptane and bicyclo[3.2.1]octane).

As used herein the term “bridged-heterocyclic ring” refers to a 5 to 10 membered heterobicyclic moiety connected at two non-adjacent ring atoms of the heterocycle containing at least one heteroatom (e.g., oxygen, sulfur, nitrogen or combinations thereof) within a 5 to 10 membered cyclic ring system. Examples of the “bridged-heterocyclic ring” include, but are not limited to, 2-oxabicyclo[2.1.1]hexane, 3-oxabicyclo[4.1.0]heptane, 2-oxabicyclo[2.2.1]heptane, 2-oxabicyclo[2.2.2]octane, 8-oxabicyclo[3.2.1]octane, and 2,6-dioxabicyclo[3.2.1]octane.

The term “heteroaryl” refers to aromatic moieties containing at least one heteroatom (e.g., oxygen, sulfur, nitrogen or combinations thereof) within a 5- to 6-membered aromatic ring system (e.g., pyrrolyl, pyridyl, pyrazolyl, thienyl, furanyl, oxazolyl, imidazolyl, tetrazolyl, triazinyl, pyrimidyl, pyrazinyl, thiazolyl, and the like) or within a 9- to 10-membered aromatic ring system (e.g., indolyl, indazolyl, benzofuranyl, quinoxalinyl and the like).

The term “5 to 6 membered heteroaryl” or “C₅₋₆ heteroaryl” refers to an aromatic moieties containing at least one heteroatom (e.g., oxygen, sulfur, nitrogen or combinations thereof) within a 5- to 6-membered monocyclic aromatic ring system. In some embodiments, a 5 to 6 membered heteroaryl is selected from pyrrolyl, pyridyl, pyrazolyl, thienyl, furanyl, oxazolyl, isoxazolyl, isothiazolyl, thiazolyl, imidazolyl, tetrazolyl, triazinyl, pyrimidyl, pyrazinyl, and thiazolyl. In some embodiments, a 5 to 6 membered heteroaryl is selected from pyridinyl, pyrimidinyl, 2H-1,2,3-triazolyl, isoxazolyl, isothiazolyl, thiazolyl, pyrazolyl and thienyl.

The term “9 to 10 membered heteroaryl” or “C₉₋₁₀ heteroaryl” refers to aromatic moieties containing at least one heteroatom (e.g., oxygen, sulfur, nitrogen or combinations thereof) within a 9- to 10-membered fused aromatic ring system. In some embodiments, a “9 to 10 membered heteroaryl” is selected from indolyl, indazolyl, benzofuranyl, quinoxalinyl, pyrazolo[1,5-a]pyridinyl, [1,2,4]triazolo[4,3-a]pyridinyl, isothiazolo[4,3-b]pyridinyl, pyrazolo[1,5-a]pyrimidinyl, pyrido[3,2-d]pyrimidinyl, imidazo[1,2-b]pyridazinyl, thieno[2,3-b]pyrazinyl, 1H-benzo[d]imidazolyl, benzo[d]thiazolyl, 1,6-naphthyridinyl, and 1,5-naphthyridinyl. In some embodiments, a “9 to 10 membered heteroaryl” is selected from pyrazolo[1,5-a]pyridinyl, [1,2,4]triazolo[4,3-a]pyridinyl, isothiazolo[4,3-b]pyridinyl, pyrazolo[1,5-a]pyrimidinyl, pyrido[3,2-d]pyrimidinyl, imidazo[1,2-b]pyridazinyl, thieno[2,3-b]pyrazinyl, 1H-benzo[d]imidazolyl, benzo[d]thiazolyl, 1,6-naphthyridinyl, 1,5-naphthyridinyl, and 2H-indazolyl.

The phrase “pharmaceutically acceptable” indicates that the substance, composition or dosage form must be compatible chemically and/or toxicologically, with the other ingredients comprising a formulation, and/or the mammal being treated therewith.

Unless specified otherwise, the term “compounds of the present invention” refers to compounds of formula (I′) or (I), as well as all stereoisomers (including diastereoisomers and enantiomers), rotamers, tautomers, isotopically labeled compounds (including deuterium substitutions), and inherently formed moieties (e.g., polymorphs, solvates and/or hydrates). When a moiety is present that is capable of forming a salt, then salts are included as well, in particular pharmaceutically acceptable salts.

As used herein, the term “a,” “an,” “the” and similar terms used in the context of the present invention (especially in the context of the claims) are to be construed to cover both the singular and plural unless otherwise indicated herein or clearly contradicted by the context. The use of any and all examples, or exemplary language (e.g. “such as”) provided herein is intended merely to better illuminate the invention and does not pose a limitation on the scope of the invention otherwise claimed.

In one Embodiment, there is provided a compound of the Examples as an isolated stereoisomer wherein the compound has one stereocenter and the stereoisomer is in the R configuration.

In one Embodiment, there is provided a compound of the Examples as an isolated stereoisomer wherein the compound has one stereocenter and the stereoisomer is in the S configuration.

In one Embodiment, there is provided a compound of the Examples as an isolated stereoisomer wherein the compound has two stereocenters and the stereoisomer is in the R configuration.

In one Embodiment, there is provided a compound of the Examples as an isolated stereoisomer wherein the compound has two stereocenters and the stereoisomer is in the R S configuration.

In one Embodiment, there is provided a compound of the Examples as an isolated stereoisomer wherein the compound has two stereocenters and the stereoisomer is in the S R configuration.

In one Embodiment, there is provided a compound of the Examples as an isolated stereoisomer wherein the compound has two stereocenters and the stereoisomer is in the S configuration.

In one Embodiment, there is provided a compound of the Examples, wherein the compound has one or two stereocenters, as a racemic mixture.

It is also possible that the intermediates and compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

In one Embodiment, the invention relates to a compound of the formula (I′) or (I) as defined herein, in free form. In another Embodiment, the invention relates to a compound of the formula (I′) or (I) as defined herein, in salt form. In another Embodiment, the invention relates to a compound of the formula (I′) or (I) as defined herein, in acid addition salt form. In a further Embodiment, the invention relates to a compound of the formula (I′) or (I) as defined herein, in pharmaceutically acceptable salt form. In yet a further Embodiment, the invention relates to a compound of the formula (I′) or (I) as defined herein, in pharmaceutically acceptable acid addition salt form. In yet a further Embodiment, the invention relates to any one of the compounds of the Examples in free form. In yet a further Embodiment, the invention relates to any one of the compounds of the Examples in salt form. In yet a further Embodiment, the invention relates to any one of the compounds of the Examples in acid addition salt form. In yet a further Embodiment, the invention relates to any one of the compounds of the Examples in pharmaceutically acceptable salt form. In still another Embodiment, the invention relates to any one of the compounds of the Examples in pharmaceutically acceptable acid addition salt form.

Furthermore, the compounds of the present invention, including their salts, may also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

Compounds of the invention, i.e. compounds of formula (I′) or (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I′) or (I).

The compounds of the present invention, including salts, hydrates and solvates thereof, may inherently or by design form polymorphs.

Compounds of the present invention may be synthesized by synthetic routes that include processes analogous to those well-known in the chemical arts, particularly in light of the description contained herein. The starting materials are generally available from commercial sources such as Sigma-Aldrich or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

The further optional reduction, oxidation or other functionalization of compounds of formula (I) may be carried out according to methods well known to those skilled in the art. Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, and in H.-D. Jakubke and H. Jeschkeit, “Aminosauren, Peptide, Proteine” (Amino acids, Peptides, Proteins), Verlag Chemie, Weinheim, Deerfield Beach, and Basel 1982. A characteristic of protecting groups is that they can be removed readily (i.e. without the occurrence of undesired secondary reactions) for example by solvolysis, reduction, photolysis or alternatively under physiological conditions (e.g. by enzymatic cleavage).

Salts of compounds of the present invention having at least one salt-forming group may be prepared in a manner known to those skilled in the art. For example, acid addition salts of compounds of the present invention are obtained in customary manner, e.g. by treating the compounds with an acid or a suitable anion exchange reagent. Salts can be converted into the free compounds in accordance with methods known to those skilled in the art. Acid addition salts can be converted, for example, by treatment with a suitable basic agent.

Any resulting mixtures of isomers can be separated on the basis of the physicochemical differences of the constituents, into the pure or substantially pure geometric or optical isomers, diastereomers, racemates, for example, by chromatography and/or fractional crystallization.

For those compounds containing an asymmetric carbon atom, the compounds exist in individual optically active isomeric forms or as mixtures thereof, e.g. as racemic or diastereomeric mixtures. Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a commercially available chiral HPLC column.

The invention further includes any variant of the present processes, in which the reaction components are used in the form of their salts or optically pure material. Compounds of the invention and intermediates can also be converted into each other according to methods generally known to those skilled in the art.

For illustrative purposes, the reaction schemes depicted below provide potential routes for synthesizing the compounds of the present invention as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### General Methods

The compounds of the Examples were analyzed or purified according to one of the Purification Methods referred to below unless otherwise described.

Where preparative TLC or silica gel chromatography have been used, one skilled in the art may choose any combination of solvents to purify the desired compound. Silica gel column chromatography was performed using 20-40 μM (particle size), 250-400 mesh, or 400-632 mesh silica gel using either a Teledyne ISCO Combiflash RF or a Grace Reveleris X₂ with ELSD purification systems or using pressurized nitrogen (˜10-15 psi) to drive solvent through the column (“flash chromatography”).

Wherein an SCX column has been used, the eluant conditions are MeOH followed by methanolic ammonia.

Except where otherwise noted, reactions were run under an atmosphere of nitrogen. Where indicated, solutions and reaction mixtures were concentrated by rotary evaporation under vacuum.

### Anaytical Methods

ESI-MS data (also reported herein as simply MS) were recorded using Waters System (Acquity HPLC and a Micromass ZQ mass spectrometer); all masses reported are the m/z of the protonated parent ions unless recorded otherwise.

### LC/MS:

A sample is dissolved in a suitable solvent such as MeCN, dimethyl sulfoxide (DMSO), or MeOH and is injected directly into the column using an automated sample handler. The analysis used_one of the following methods: (1) acidic method (1.5, 2, 3.5, 4, or 7_min runs, see Acidic LCMS section for additional details vide infra: conducted on a Shimadzu 2010 Series, Shimadzu 2020 Series, or Waters Acquity UPLC BEH. (MS ionization: ESI) instrument equipped with a C18 column (2.1 mm×30 mm, 3.0 mm or 2.1 mm×50 mm, C18, 1.7 μm), eluting with 1.5 mL/4 L of trifluoroacetic acid (TFA) in water (solvent A) and 0.75 mL/4 L of TFA in MeCN (solvent B) or (2) basic method (3, 3.5, 7 min runs, see Basic LCMS section for additional details vide infra: conducted on a Shimadzu 2020 Series or Waters Acquity UPLC BEH (MS ionization: ESI) instrument equipped with)(Bridge Shield RP18, 5 um column (2.1 mm×30 mm, 3.0 mm i.d.) or 2.1 mm×50 mm, C18, 1.7 μm column, eluting with 2 mL/4 L NH₃.H₂O in water (solvent A) and MeCN (solvent B).

The invention further includes any variant of the present processes, in which the reaction components are used in the form of their salts or optically pure material. Compounds of the invention and intermediates can also be converted into each other according to methods generally known to those skilled in the art.

### Analytical HPLC

Acidic HPLC: Conducted on a Shimadza 20A instrument with an ultimate C18 3.0 x 50 mm, 3 μm column eluting with 2.75 mL/4 L TFA in water (solvent A) and 2.5 mL/4 L TFA in acetonitrile (solvent B) by the following methods:

Method A: using the following elution gradient 0%-60% (solvent B) over 6 minutes and holding at 60% for 2 minutes at a flow rate of 1.2 ml/minutes. Wavelength: UV 220 nm, 215 nm and 254 nm.

Method B: using the following elution gradient 10%-80% (solvent B) over 6 minutes and holding at 60% for 2 minutes at a flow rate of 1.2 ml/minutes. Wavelength: UV 220 nm, 215 nm and 254 nm.

Method C: using the following elution gradient 30%-90% (solvent B) over 6 minutes and holding at 60% for 2 minutes at a flow rate of 1.2 ml/minutes. Wavelength: UV 220 nm, 215 nm and 254 nm.

Basic HPLC: Conducted on a Shimadza 20A instrument with Xbrige Shield RP-18, 5 um, 2.1×50 mm column eluting with 2 mL/4 L NH3H2O in water (solvent A) and acetonitrile (solvent B), by the following methods:

Method D: using the following elution gradient 0%-60% (solvent B) over 4.0 minutes and holding at 60% for 2 minutes at a flow rate of 1.2 ml/minutes.

Method E: using the following elution gradient 10%-80% (solvent B) over 4.0 minutes and holding at 60% for 2 minutes at a flow rate of 1.2 ml/minutes.

Method F: using the following elution gradient 30%-90% (solvent B) over 4.0 minutes and holding at 60% for 2 minutes at a flow rate of 1.2 ml/minutes.

### Analytical LCMS

Acidic LCMS: Conducted on a Shimadza 2010 Series, Shimadza 2020 Series, or Waters Acquity UPLC BEH. (MS ionization: ESI) instrument equipped with a C18 column (2.1 mm×30 mm, 3.0 mm or 2.1 mm×50 mm, C18, 1.7 μm), eluting with 1.5 mL/4 L TFA in water (solvent A) and 0.75 mL/4 LTFA in acetonitrile (solvent B) using the methods below:

1.5 minute methods:

General method: using the following elution gradient 5%-95% (solvent B) over 0.7 minutes and holding at 95% for 0.4 minutes at a flow rate of 1.5 ml/minutes. Wavelength: UV 220 nm and 254 nm.

2 minute methods:

Method A: using the following elution gradient 0%-60% (solvent B) over 0.9 minutes and holding at 60% for 0.6 minutes at a flow rate of 1.2 ml/minutes. Wavelength: UV 220 nm and 254 nm.

Method B: using the following elution gradient 10%-80% (solvent B) over 0.9 minutes and holding at 60% for 0.6 minutes at a flow rate of 1.2 ml/minutes. Wavelength: UV 220 nm and 254 nm.

Method C: using the following elution gradient 30%-90% (solvent B) over 0.9 minutes and holding at 60% for 0.6 minutes at a flow rate of 1.2 ml/minutes. Wavelength: UV 220 nm and 254 nm.

3.5 minute method:

Initial conditions, solvent A-95%: solvent B-5%; hold at initial from 0.0-0.1 min; Linear Ramp to solvent A-5%: solvent B-95% between 0.1-3.25 min; hold at solvent A-5%:solvent B-95% between 3.25-3.5 min. Diode array/MS detection.

4 minute methods:

Method A: using the following elution gradient 0%-60% (solvent B) over 3 minutes and holding at 60% for 0.5 minutes at a flow rate of 0.8 ml/minutes. Wavelength: UV 220 nm and 254 nm.

Method B: using the following elution gradient 10%-80% (solvent B) over 3 minutes and holding at 60% for 0.5 minutes at a flow rate of 0.8 ml/minutes. Wavelength: UV 220 nm and 254 nm.

Method C: using the following elution gradient 30%-90% (solvent B) over 3 minutes and holding at 60% for 0.5 minutes at a flow rate of 0.8 ml/minutes. Wavelength: UV 220 nm and 254 nm.

7 minute methods:

Method A: using the following elution gradient 0%-60% (solvent B) over 6 minutes and holding at 60% for 0.5 minutes at a flow rate of 0.8 ml/minutes. Wavelength: UV 220 nm and 254 nm.

Method B: using the following elution gradient 10%-80% (solvent B) over 6 minutes and holding at 60% for 0.5 minutes at a flow rate of 0.8 ml/minutes. Wavelength: UV 220 nm and 254 nm.

Method C: using the following elution gradient 30%-900% (solvent B) over 6 minutes and holding at 60% for 0.5 minutes at a flow rate of 0.8 ml/minutes. Wavelength: UV 220 nm and 254 nm.

Basic LCMS: Conducted on a Shimadza 2020 Series or Waters Acquity UPLC BEH (MS ionization: ESI) instrument equipped with)(Bridge Shield RP18, Sum column (2.1 mm×30 mm, 3.0 mm i.d.) or 2.1 mm×50 mm, C18, 1.7 μm column, eluting with 2 mL/4 L NH3H2O in water (solvent A) and acetonitrile (solvent B) using the methods below:

3 minute methods:

Method A: using the following elution gradient 0%-60% (solvent B) over 2 minutes and holding at 60% for 0.48 minutes at a flow rate of 1 ml/minutes. Wavelength: UV 220 nm and 254 nm.

Method B: using the following elution gradient 10%-80% (solvent B) over 2 minutes and holding at 60% for 0.48 minutes at a flow rate of 1 ml/minutes. Wavelength: UV 220 nm and 254 nm.

Method C: using the following elution gradient 30%-90% (solvent B) over 2 minutes and holding at 60% for 0.48 minutes at a flow rate of 1 ml/minutes. Wavelength: UV 220 nm and 254 nm.

3.5 minute method:

Initial conditions, solvent A-95%: solvent B-5%; hold at initial from 0.0-0.1 min; Linear Ramp to solvent A-5%: solvent B-95% between 0.1-3.25 min; hold at solvent A-5%:solvent B-95% between 3.25-3.5 min. Diode array/MS detection.

7 minute methods:

Method A: using the following elution gradient 0%-60% (solvent B) over 6 minutes and holding at 60% for 0.5 minutes at a flow rate of 0.8 ml/minutes. Wavelength: UV 220 nm and 254 nm.

Method B: using the following elution gradient 10%-80% (solvent B) over 6 minutes and holding at 60% for 0.5 minutes at a flow rate of 0.8 ml/minutes. Wavelength: UV 220 nm and 254 nm.

Method C: using the following elution gradient 30%-90% (solvent B) over 6 minutes and holding at 60% for 0.5 minutes at a flow rate of 0.8 ml/minutes. Wavelength: UV 220 nm and 254 nm.

### SFC Analytical Separation

Instrument: Waters UPC2 analytical SFC (SFC—H). Column: ChiralCel OJ, 150×4.6 mm I.D., 3 μm. Mobile phase: A for CO2 and B for Ethanol (0.05% DEA). Gradient: B 40%. Flow rate: 2.5 mL/min. Back pressure: 100 bar. Column temperature: 35° C. Wavelength: 220 nm

### Preparative HPLC Purification

General Method: Preparative HPLC was performed on a Gilson UV/VIS-156 with UV detection at 220/254 nm Gilson 281 automatic collection.

Acidic condition: Two acid grading systems used: Hydrochloride acid and Formic acid.

Method A: Hydrochloride acid: YMC-Actus Triart C18 150×30 mm×5 um, Gradient used 0-100% acetonitrile with water and corresponding acid (0.05% HCl).

Method B: Formic acid: Phenomenex Synergi C18 150×30 mm×4 um, Gradient used 0-100% acetonitrile with water and corresponding acid (0.225% formic acid), the gradient shape was optimized for individual separations.

Neutral condition: Xtimate C18 150×25 mm×5 um, Gradient used 0-100% (water (10 mM NH₄HCO₃)-ACN), the gradient shape was optimized for individual separations.

Basic condition: Waters Xbridge Prep OBD C18 150×30 10 um, Gradient used 0-100% water (0.04% NH₃H₂O+10 mM NH₄HCO₃)-acetonitrile, the gradient shape was optimized for individual separations.

### Preparative HPLC Conditions

### Column: Phenomenex Synergi C18 150×30 mm; 4

Mobile phase A: MeCN
Mobile phase B: H₂O

### Modifier: 0.225% HCO₂H

Gradient (% organic): 0-100% optimised for each example

### Column: Sunfire C18 100×19 mm, 5 μm

Mobile phase A: MeCN
Mobile phase B: H₂O

### Modifier: 0.1% TFA

Gradient (% organic): 5-95% optimised for each example.

### Column: Sunfire C18 100×19 mm, 5 μm

Mobile phase A: MeCN
Mobile phase B: H₂O
Gradient (% organic): 5-95% optimised for each example.

### Column: XBridge C18 100×19 mm; 5 μm

Mobile phase A: MeCN
Mobile phase B: H₂O

### Modifier: 0.1% NH₄OH

Gradient (% organic): 0-100% optimised for each example.

### Column: XSelect C18 50×30 mm; 5 μm

Mobile phase A: MeCN
Mobile phase B: H₂O

### Modifier: 0.1% NH₄OH

Gradient (% organic): 0-100% optimised for each example.
Detectors: Gilson UV/VIS-156 with UV detection at 220/254 nm, Gilson 281 automatic collection, utilizing acidic, basic and neutral methods. For mass-directed peak collection, an ACQUITY QDa Mass Detector (Waters Corporation) was employed.

### Preparative SFC Purification

Instrument: MG III preparative SFC (SFC-1). Column: ChiralCel OJ, 250×30 mm I.D., 5 μm. Mobile phase: A for CO2 and B for Ethanol (0.1% NH3H2O). Gradient: B 50%. Flow rate: 40 mL/min. Back pressure: 100 bar. Column temperature: 38° C. Wavelength: 220 nm. Cycle time: ˜8 min.

Column: Chiralpak AD-H; 250 mm×30 mm, 5 μm; 40% (EtOH+0.1% DEA)/CO₂
Column: Chiralpak IA; 250 mm×30 mm, 5 μm; 40% (MeOH+0.1% DEA)/CO₂
Column: Chiralpak IB; 250 mm×30 mm, 5 μm; 40% (EtOH+0.1% DEA)/CO₂
Column: Chiralpak AD-H; 250 mm×30 mm, 5 μm; 40% (EtOH+0.1% NH₄OH)/CO₂
Column: Chiralpak OJ-H; 250 mm×30 mm, 5 μm; 30% (EtOH+0.1% NH₄OH)/CO₂
Column: Chiralpak OD; 250 mm×30 mm, 5 μm; 35% (EtOH+0.1% NH₄OH)/CO₂

### 1H-NMR

¹H nuclear magnetic resonance (NMR) spectra were in all cases consistent with the proposed structures. The 1H NMR spectra were recorded on a Bruker Avance III HD 500 MHz, Bruker Avance III 500 MHz, Bruker Avance III 400 MHz, Varian-400 VNMRS, or Varian-400 MR. Characteristic chemical shifts (6) are given in parts-per-million downfield from tetramethylsilane (for ¹H-NMR) using conventional abbreviations for designation of major peaks: e.g. s, singlet; d, doublet; t, triplet; q, quartet; dd, double doublet; dt, double triplet; m, multiplet; br, broad. The following abbreviations have been used for common solvents: CDCl₃, deuterochloroform; DMSO-d₆, hexadeuterodimethyl sulfoxide; and MeOH-d₄, deuteron-methanol. Where appropriate, tautomers may be recorded within the NMR data; and some exchangeable protons may not be visible.

Typically, the compounds of Formula (I) can be prepared according to the schemes provided below. The following examples serve to illustrate the invention without limiting the scope thereof. Methods for preparing such compounds are described hereinafter

Abbreviations:

Abbreviations used are those conventional in the art or the following:

| | |
|---|---|
| AcOH means Acetic acid; | Min(s): minute(s) |
| Aq. means aqueous; | m/z: mass to charge ratio |
| Ar means argon; | Bn means benzyl; |
| BINAP means (±)-2,2′- | Boc means tert-butoxy carbonyl; |
| Bis(diphenylphosphino)-1,1′- | |
| binaphthalene; | |
| LC and LCMS: liquid | MeOH: methanol |
| chromatography and liquid | |
| chromatography-mass spectrometry | |
| br means broad; | nBuOH means n-butanol; |
| tBuOH means tert butanol; | n-BuLi means n-butyl lithium; |
| HRMS: high resolution mass | Pd₂(dba)₃ means |
| spectrometry | Tris(dibenzylideneacetone)dipalladium(0) |
| ° C. means degrees Celsius; | CHCl₃ means chloroform; |
| CDCl₃ means deutero-chloroform; | CDI means 1,1′-carbonyldiimidazole; |
| ESI: electrospray ionization | MeCN: acetonitrile |
| CO means carbon monoxide; | (COCl)₂ means oxalyl chloride; |
| Cs₂CO₃ means cesium carbonate; | δ means chemical shift; |
| d means doublet; | dd means double doublet; |
| DABAL-Me₃ means | DMSO-d₆ means hexadeuterodimethyl |
| bis(trimethylaluminium)-1,4- | sulfoxide; |
| diazabicyclo[2.2.2]octane adduct; | |
| DCM: dichloromethane | DMAP means 4-(dimethylamino)pyridine; |
| Et means ethyl; | DMF: dimethylformamide |
| Et₂O means diethyl ether; | |
| EtOH: ethanol | EtOAc means ethyl acetate; |
| Equiv. means equivalent; | DMSO: dimethylsulfoxide |
| g means gram; | F-TEDA means N-Chloromethyl-N′- |
| | fluorotriethylenediammonium |
| | bis(tetrafluoroborate); |
| HATU means 1- | HBr means hydrogen bromide; |
| [bis(dimethylamino)methylene]-1H- | |
| 1,2,3-triazolo[4,5-b]pyridinium 3- | |
| oxid hexafluorophosphate; | |
| Na₂SO₃: sodium sulfite; | Pd(OAc)₂: Palladium(II) acetate |
| HCl means hydrochloric acid; | HCO₂H means formic acid; |
| Hex means hexane; | |
| ¹HNMR means proton nuclear | HOAt means 1-hydroxy-7-azabenzotriazole; |
| magnetic resonance; | |
| DIPEA: diisopropyl ethylamine | SCX: strong cation exchange sorbent, solid phase |
| | purification reagent |
| T3P ®: 2,4,6-Tripropyl-1,3,5,2,4,6- | N2 or N₂ means nitrogen |
| trioxatriphosphorinane-2,4,6-trioxide | |
| solution | |
| HPLC means high pressure liquid | hr means hour; |
| chromatography; | |
| K₂CO₃ means potassium carbonate; | mL means millilitres; |
| KHSO₄ means potassium bisulfate; | mins means minutes; |
| KI means potassium iodide; | mmol means millimole; |
| KOH means potassium hydroxide; | Mukaiyama's reagent means 2-chloro-1- |
| | methylpyridinium iodide; |
| K₂OsO₄ means potassium | MTBE means tert-butyl methyl ether; |
| osmate(VI); | |
| L means litre; | M/V means Mass volume ratio; |
| LCMS means liquid chromatography | |
| mass spectrometry; | |
| LiBr means lithium bromide; | |
| LiOH means lithium hydroxide; | NaBH₃CN means sodium cyanoborohydride; |
| m means multiplet | MsCl means methanesulfonyl chloride; |
| Na means sodium; | NCS means N-chlorosuccinimide; |
| NaOEt means sodium ethoxide; | |
| M means molar; | Na₂CO₃ means sodium carbonate; |
| Me means methyl; | NaH means sodium hydride; |
| MeCN means acetonitrile; | NaHCO₃ means sodium bicarbonate; |
| MeOH means methanol; | NaI means sodium iodide; |
| MeOH-d₄ means deutero-methanol; | NaOH means sodium hydroxide; |
| mg means milligram; | Na₂SO₄ means sodium sulfate; |
| MgSO₄ means magnesium sulfate; | NH₃ means ammonia; |
| MS m/z means mass spectrum peak; | NH₄Cl means ammonium chloride; |
| NH₄HCO₃ means ammonium | NH₄OH is ammonium hydroxide; |
| bicarbonate; | |
| OMs means mesylate; | PE means petroleum ether; |
| OTs means tosylate; | Pd(dppf)Cl₂ means [1,1′- |
| | bis(diphenylphosphino)ferrocene]dichloropalladium(II); |
| Pd(OAc)₂ means palladium acetate; | PrCN means butyronitrile; |
| Pd(PPh₃)₄ means | rt means room temperature; |
| tetrakis(triphenylphosphine)palladium(0); | |
| q means quartet; | sat. means saturated; |
| s means singlet; | soln. means solution; |
| SFC means supercritical fluid | t means triplet; |
| chromatography; | |
| STAB means sodium | |
| triacetoxyborohydride; | |
| TFA means trifluoroacetic acid; | t-BuONa means sodium tert-butoxide; |
| TEA means triethylamine; | TBDMS means tert-butyldimethylsilyl; |
| TBAF means tetrabutylammonium | T3P ® means propylphosphonic anhydride solution; |
| fluoride; | |
| TLC means thin layer | THF means tetrahydrofuran; |
| chromatography; | |
| TMSCHN₂ means | TMS means trimethylsilyl; |
| (trimethylsilyl)diazomethane; | |
| μmol means micromole; | μL means micro litres; |
| Xantphos means 4,5- | XPhos means 2-dicyclohexylphosphino-2′,4′,6′- |
| bis(diphenylphosphino)-9,9- | triisopropylbiphenyl; |
| dimethylxanthene; | |
| XantPhos-Pd-G3 means [(4,5- | D₂O means deuterated water; |
| Bis(diphenylphosphino)-9,9- | |
| dimethylxanthene)-2-(2′-amino-1,1′- | |
| biphenyl)]palladium(II) | |
| methanesulfonate; | |
| BOP: (Benzotriazol-1- | NBS: N-bromosuccinimide |
| yloxy)tris(dimethylamino)phosphonium | |
| hexafluorophosphate | |
| Zn(CN)₂ means zinc cyanide; | ABPR: Automated Back Pressure Regulator |
| MBPR: manual back pressure regular | DEA: diethylamine |
| PE: petroleum ether | MHz means mega Hertz; |
| NIS: N-Iodosuccinimide | TFA: 2,2,2-trifluoroacetic acid |
| NaHMDS: Sodium | t-BuOK: Potassium t-butoxide |
| bis(trimethylsilyl)amide | |

For illustrative purposes, the reaction schemes depicted below provide potential routes for synthesizing the compounds of the present invention as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

Schemes

Scheme 1,2,3,4, and 5 provide potential routes for making compounds of Formula (I).

Scheme 1:

According to a first process, compounds of Formula (I), may be prepared from compounds of Formulae (II) and (III) as illustrated by Scheme 1.

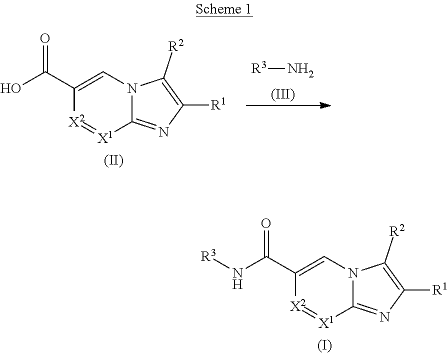

The compound of Formula (I) may be prepared by an amide bond formation of the acid of Formula (II) and the amine of Formula (III) in the presence of a suitable coupling agent and organic base in a suitable polar aprotic solvent. Preferred conditions, comprise reaction of the acid of Formula (II) with the amine of Formula (III) in the presence of a coupling agent preferably, T3P®, HATU, CDI, HOAt in the presence of EDC, Mukaiyama's reagent, or MsCl, optionally in the presence of N-methyl imidazole, in the presence of a suitable organic base such as TEA, DIPEA or pyridine, or strong base such as tBuONa, optionally in a suitable solvent, such as DMF, DMSO, EtOAc or MeCN at between rt and the reflux temperature of the reaction and optionally in the presence of microwave irradiation.

Scheme 2:

According to a second process, compounds of Formula (II), may be prepared from compounds of Formulae (IV), (V), (VI), (VII), (VIII) and (IX) as illustrated by Scheme 2.

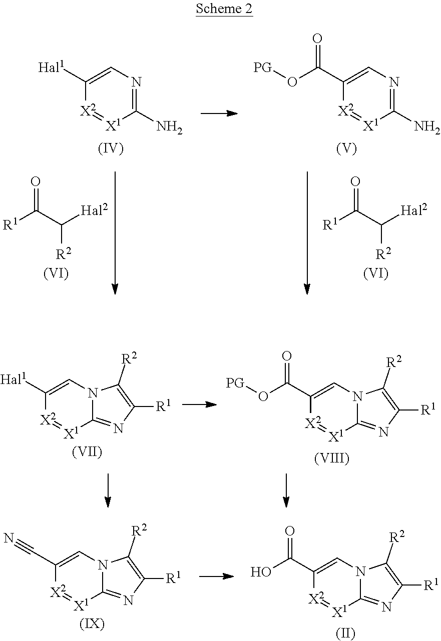

Hal¹ is halogen, preferably Br or I

Hal² is halogen, preferably Cl or Br

PG is a carboxylic acid protecting group, typically C₁-C₄ alkyl or phenyl and preferably Me, Et, isopropyl or phenyl.

Compounds of Formula (V) may be prepared from the bromide of Formula (IV) by a palladium catalysed carbonylation reaction, in the presence of a suitable palladium catalyst, organic base and suitable alcohol at elevated temperature under an atmosphere of CO. When PG is methyl or ethyl, preferred conditions comprise, reaction of the bromide of Formula (IV) under an atmosphere of CO in the presence of suitable palladium catalyst such as Pd(dppf)Cl₂ or Pd(OAc)₂ with a phosphine-based ligand such PPh₃, an organic base such as TEA in a solvent such as MeOH or EtOH at between 80 and 100° C. Alternatively, when PG is phenyl, compounds of Formula (V) may be prepared from the bromide of Formula (IV) by a palladium catalyzed reaction with phenyl formate, in the presence of a suitable palladium catalyst such as such as Xantphos Pd-G3, or a suitable palladium catalyst such as Pd(OAc)₂ with a phosphine-based ligand such as BINAP or XantPhos, an organic base such as TEA, in a solvent such as MeCN at between 80 and 100° C.

Compounds of Formula (VII) may be prepared from the amine of Formula (IV) and the haloketone of Formula (VI) by a condensation/cyclisation reaction. Preferred conditions comprise reaction of the amine of Formula (IV) with the haloketone of Formula (VI) optionally in the presence of a suitable inorganic base such as K₂CO₃ or NaHCO₃ and optionally in the presence of a catalyst such as KI, in a suitable protic solvent such as MeOH, EtOH, n-BuOH, t-BuOH, MeCN or MeCN/toluene at elevated temperature, typically between 60 to 100° C.

Compounds of Formula (VIII) may be prepared from the amine of Formula (V) and the haloketone of Formula (VI) by a condensation/cyclisation reaction as described above.

Alternatively, compounds of Formula (VIII) may be prepared from the bromide of Formula (VII) by a palladium catalysed carbonylation reaction as described above.

Compounds of Formula (IX) may be prepared from the bromide of Formula (VII) by a palladium catalysed cyanation reaction, in the presence of a suitable palladium catalyst, a suitable cyanide source in a polar aprotic solvent at elevated temperature. Preferred conditions comprise, reaction of the bromide of Formula (VII) with Zn(CN)₂, in the presence of Pd(PPh₃)₄, in DMF at about 120° C.

Compounds of Formula (II) may be prepared by the hydrolysis of the ester of Formula (VIII) under suitable acidic or basic conditions in a suitable aqueous solvent. Preferred conditions comprise the treatment of the ester of Formula (VIII) with an alkali metal base such as LiOH, NaOH, K₂CO₃ or Na₂CO₃ in aqueous MeOH and/or THF at between rt and the reflux temperature of the reaction.

Alternatively, compounds of Formula (II) may be prepared from the hydrolysis of the compounds of Formula (IX) under suitable acidic or basic conditions in a suitable aqueous solvent. Preferred conditions comprise treatment of the nitrile of Formula (XI) with an alkali metal hydroxide such as LiOH or NaOH in aqueous MeOH at the reflux temperature of the reaction.

Scheme 3:

According to a third process, compounds of Formula (I), may be prepared from compounds of Formulae (III), (VI), (X), (XI), (XII), (XIII) and (XIV) as illustrated by Scheme 3.

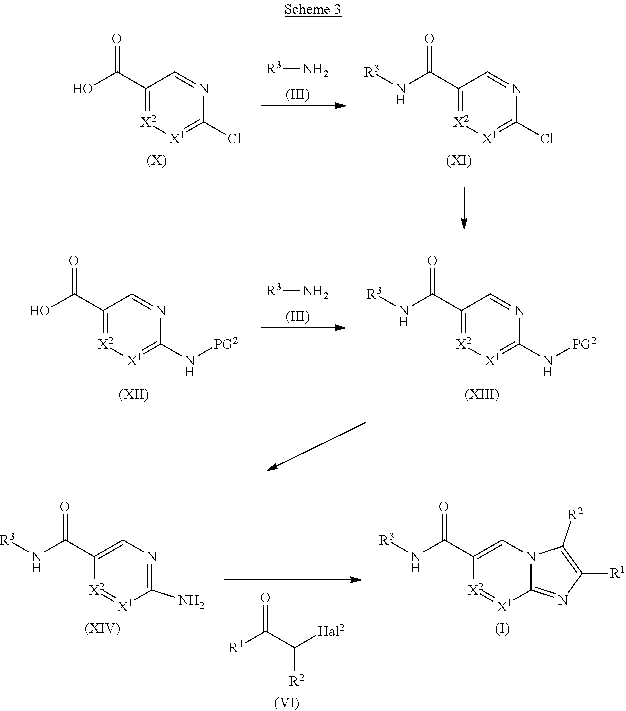

Hal² is halo, preferably Cl or Br

PG² is a NH protecting group, typically a carbamate and preferably Boc.

The compound of Formula (XI) may be prepared by an amide bond formation of the acid of Formula (X) and the amine of Formula (III) in the presence of a suitable coupling agent and organic base in a suitable polar aprotic solvent. Preferred conditions, comprise the reaction of the acid of Formula (X) with the amine of Formula (III) in the presence of HATU or T3P®, in the presence of a suitable organic base, typically DIPEA in a suitable solvent, such as DMF or EtOAc at rt.

Alternatively, this coupling may be achieved, via the in-situ formation of the acid chloride of the acid of Formula (X), typically using oxalyl chloride and DMF in DCM at rt and the subsequent reaction with the amine of Formula (III) in the presence of a suitable organic base, typically TEA at between 0° C. and rt.

The compound of Formula (XIII) may be prepared from the chloride of Formula (XI) and the compound of Formula NH₂PG² via an amination reaction under Buchwald-Hartwig cross-coupling conditions. Typical conditions comprise, reaction of the compound of Formula (XI) with NH₂PG² in the presence of a suitable palladium catalyst such as Pd(OAc)₂, a phosphine-based ligand such as BINAP or XantPhos and a suitable inorganic base such as Cs₂CO₃ in a suitable solvent such as dioxane at between rt and 110° C.

Alternatively, compounds of Formula (XIII) may be prepared from the acid of Formula (XII) and the amine of Formula (III) by an amide coupling reaction as previously described in Scheme 1.

The amine of Formula (XIV) may be prepared by a suitable deprotection reaction, typically involving treatment of the compound of Formula (XIII) with an acid such as HCl or TFA in a suitable aprotic solvent such as DCM or dioxane at between rt and reflux temperature. Preferred conditions comprise, reaction of the compound of Formula (XIII) with TFA in DCM at rt.

Compounds of Formula (I) may be prepared from the amine of Formula (XIV) and the haloketone of Formula (VI) in the presence of an inorganic base and a suitable polar solvent at elevated temperature. Preferred conditions comprise reaction of the amine of Formula (XIV) and the haloketone of Formula (VI) in the presence of Na₂CO₃ or NaHCO₃ in a suitable solvent such as EtOH, MeCN, PrCN and toluene or dioxane, at between 80 and 100° C.

Scheme 4:

According to a fourth process, compounds of Formula (I) may be prepared directly from compounds of Formula (VIII) as illustrated in Scheme 4.

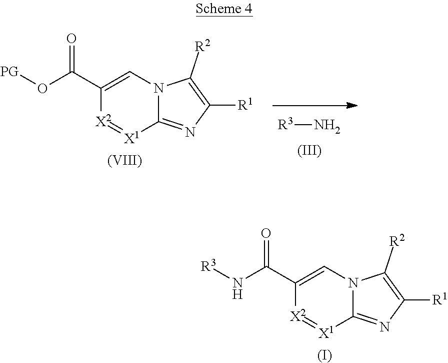

PG is a protecting group, as previously defined in Scheme 2

The compound of Formula (I) may be prepared from the ester of Formula (VIII) by reaction with a strong base in a suitable polar aprotic solvent to form the carboxylate ion in-situ, followed by reaction with the amine of Formula (III). Preferred conditions comprise treatment of the ester of Formula (VIII) with n-BuLi at low temperature (−80° C.) in a solvent, typically THF, followed by reaction of the amine of Formula (III) at between −80° C. and rt.

Alternatively, the compound of Formula (I) may be prepared from the ester of Formula (VIII) by reaction of the amine of Formula (III) in the presence of a suitable coupling agent, typically DABAL-Me₃ according to the method described by Novak et al (Tet. Lett. 2006, 47, 5767).

Scheme 5:

According to a fifth process, compounds of Formula (XIV), may be prepared from compounds of Formula (XV) as illustrated by Scheme 5.

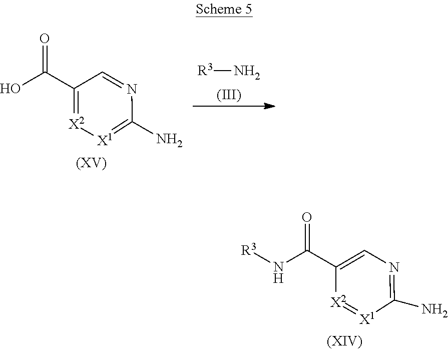

The compound of Formula (XIV) may be prepared from the acid of Formula (XV) and the amine of Formula (III) by an amide coupling reaction as previously described in Scheme 1.

Compounds of Formulae (I), (V), (VII), (VIII), (IX), (XI), (XIII) and (XIV) may be converted to alternative compounds of Formulae (I), (V), (VII), (VIII), (IX), (XI), (XIII) and (XIV) by standard chemical transformations such as for example, alkylation of a heteroatom such as N or O, halogenation, such as chlorination or fluorination, palladium catalysed cross-coupling reactions, transesterification reactions, using methods well known to those skilled in the art.

For example, see Preparation 62, Preparation 269, Examples 90, 207, 229, 435 to 478, or 640.

The compounds of Formulae (III), (IV), (V), (VI), (X), (XII) and (XV) are commercially available, may be prepared by analogy to methods known in the literature, or the methods described in the Experimental section below.

It will be appreciated by those skilled in the art that it may be necessary to utilize a suitable protecting group strategy for the preparation of compounds of Formula (I). Typical protecting groups may comprise, carbamate and preferably Boc for the protection of amines, a TBDMS or benzyl group for the protection of a primary alcohol, a C₁₋₄ alkyl, phenyl or benzyl group for the protection of carboxylic acids.

It will be appreciated by those skilled in the art that the experimental conditions set forth in the schemes that follow are illustrative of suitable conditions for effecting the transformations shown, and that it may be necessary or desirable to vary the precise conditions employed for the preparation of the compound of Formula (I). It will be further appreciated that it may be necessary or desirable to carry out the transformations in a different order from that described in the schemes, or to modify one or more of the transformations, to provide the desired compound of the invention

### PREPARATION OF INTERMEDIATES

### Preparation 1: 5-Bromo-4-isopropoxypyridin-2-amine

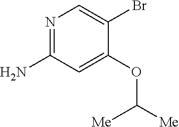 [SMILES: CC(C)Oc1cc(N)ncc1Br]

5-Bromo-4-chloro-pyridin-2-amine (50.0 g, 241.0 mmol) was added to a solution of Na (13.85 g, 602.5 mmol) in isopropanol (500 mL) and the reaction heated at 82° C. for 92 h. The reaction mixture was cooled to rt and poured into ice. The resulting precipitate was filtered off, washed with water and dried to afford the title compound as a yellow solid, 43.5 g, 76.5% yield. LCMS m/z=231 [M+H]⁺

### Preparation 2: Methyl 6-amino-4-isopropoxynicotinate

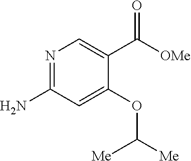 [SMILES: COC(=O)c1cnc(N)cc1OC(C)C]

A mixture of 5-bromo-4-isopropoxy-pyridin-2-amine (Preparation 1, 25.0 g, 108.2 mmol), TEA (18.0 mL, 129.8 mmol) and Pd(dppf)Cl₂ (2.37 g, 3.25 mmol) in MeOH (300 mL) was heated at 120° C. under a 40 atm. CO pressure for 48 h. The cooled mixture was concentrated in vacuo and the residue diluted with water (100 mL). The mixture was extracted with EtOAc (2×100 mL), the combined organic extracts dried over Na₂SO₄ and evaporated under reduced pressure to afford methyl 6-amino-4-isopropoxynicotinate (21.0 g, 89.5% yield) as a brown solid. LCMS m/z=211.1 [M+H]⁺. ¹H NMR (500 MHz, CDCl₃) δ: 1.38 (d, 6H), 3.81 (s, 3H), 4.55-4.59 (m, 1H), 4.97 (br s, 1H), 5.93 (s, 1H), 8.54 (s, 1H).

### Preparation 3: Methyl 6-amino-4-ethoxynicotinate

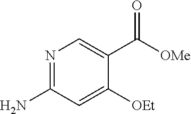 [SMILES: CCOc1cc(N)ncc1C(=O)OC]

was obtained as a light brown solid, 11.0 g, 79.3% yield, from 5-bromo-4-ethoxypyridin-2-amine, following the procedure described in Preparation 2. LCMS m/z=197.2 [M+H]^{+ 1}H NMR (400 MHz, CDCl₃): δ 1.35-1.56 (m, 3H), 3.81 (s, 3H), 4.06 (q, 2H), 4.81 (br s, 2H), 5.90 (s, 1H), 8.53 (s, 1H).

### Preparation 4: 1-Chloro-3,3-difluorobutan-2-one

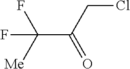

A mixture of 2,2-difluoropropanoic acid (5.0 g, 45.43 mmol) and phenylphosphonic dichloride (8.04 mL, 54.36 mmol) was stirred at 70° C. for 2 h with simultaneous distillation of the product. 2,2-Difluoropropanoyl chloride was obtained as a yellow oil, 5.10 g, 82.9% yield. TMSCHN₂ (2 M, 15 mL) was added to a solution of 2,2-difluoropropanoyl chloride (5.10 g, 39.7 mmol) in THF (25 mL) and MeCN (25 mL) at 0° C. and the reaction stirred for 1 h. HCl (12 M, 7.3 mL) was added and the reaction stirred at 30° C. for 3 h. The resulting mixture was diluted with cold water (100 mL), then basified with sat. aq. NaHCO₃ to pH=8˜9. The aqueous layer was extracted with Et₂O (3×100 mL), the combined organic layers washed with brine (100 mL), dried over Na₂SO₄, filtered and evaporated under reduced pressure to afford 1-chloro-3,3-difluorobutan-2-one, 3.10 g, 52.6% yield, as a yellow oil. ¹H NMR (500 MHz, CDCl₃) δ: 1.72-1.87 (m, 3H), 4.46-4.61 (m, 2H)

### Preparation 5: 2-Chloro-1-(2,2-difluorocyclopropyl)ethan-1-one

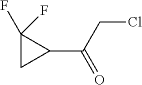

SOCl₂ (974.60 mg, 8.19 mmol) and five drops of DMF were added to a solution of 2,2-difluorocyclopropane-1-carboxylic acid (1.0 g, 8.19 mmol) in DCM (10.0 mL) at 0° C. and the reaction stirred at 0° C. for 14 h. The mixture was concentrated in vacuo, the residue diluted with THF (10.0 mL) and MeCN (6.0 mL) and the solution cooled to 0° C. TMSCHN₂ in THF (2 M, 4.10 mL) was added and the mixture stirred at 0° C. for 1 h. HCl in dioxane (4 M, 2.05 mL) was added and the reaction stirred at rt for 1 h. The reaction was quenched with saturated aq. NaHCO₃(70 mL) and the mixture extracted with EtOAc (150 mL×3). The combined organic layers were washed with brine (30 mL), dried over Na₂SO₄, filtered and evaporated under reduced pressure to afford 2-chloro-1-(2,2-difluorocyclopropyl)ethan-1-one as a yellow oil, 500 mg. ¹H NMR (400 MHz, CDCl₃) δ: 1.78-1.88 (m, 1H), 2.25-2.33 (m, 1H), 3.03-3.12 (m, 1H), 4.20 (d, 2H)

### Preparation 6: 2-Chloro-1-(1-methyl-2-oxabicyclo[2.1.1]hexan-4-yl)ethan-1-one

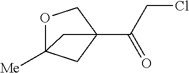 [SMILES: CC12CC(C(=O)CCl)(CO1)C2]

Oxalyl chloride (178.6 μL, 2.11 mmol) was added dropwise to a mixture of 1-methyl-2-oxabicyclo[2.1.1]hexane-4-carboxylic acid (250.0 mg, 1.76 mmol) in DCM (4.0 mL) containing one drop of DMF at 0° C., and the reaction stirred for 3 h. The mixture was concentrated in vacuo, the crude product dissolved in THF (4 mL) and the solution cooled to 0° C. TMSCHN₂ (2 M, 1.14 mL) was added dropwise, the mixture stirred at 0° C. for 1 h, then at rt for a further 14 h. The reaction was re-cooled to 0° C., HCl (12 M, 440.0 μL) added and the solution stirred for 1 h. The mixture was neutralized using sat. aq. NaHCO₃ then extracted with EtOAc (20 mL×3) and the combined organic layers washed with brine (50 mL), dried over Na₂SO₄ and filtered. The filtrate was evaporated under reduced pressure to afford 2-chloro-1-(1-methyl-2-oxabicyclo[2.1.1]hexan-4-yl)ethan-1-one. ¹H NMR (400 MHz, CDCl₃) δ: 1.47 (s, 3H), 1.90-1.94 (m, 2H), 2.02-2.06 (m, 2H), 3.98 (s, 2H), 4.23 (s, 2H)

### Preparations 7 to 33

The compounds in the following table were prepared from the appropriate acid, following the procedure described in Preparation 6.

| Prep. | | |
|---|---|---|
| No | Structure and Name | Starting Material |
| 7 | 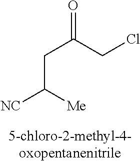 | 3-cyano-3- methylpropanoic acid |
| 8 | 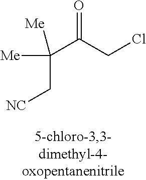 | 3-cyano-2,2- dimethylpropanoic acid |
| 9 | 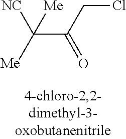 | 2-cyano-2-methylpropanoic acid |
| 10 | 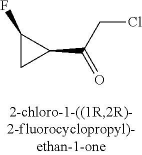 | (1R,2R)-2- fluorocyclopropane-1- carboxylic acid |
| 11 | 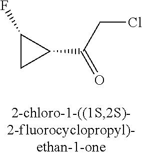 | (1S,2S)-2- fluorocyclopropane-1- carboxylic acid |
| 12 | 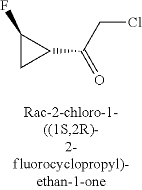 | Rac-(1S,2R)-2- fluorocyclopropane-1- carboxylic acid |
| 13 | 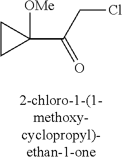 | 1-methoxycyclopropane-1- carboxylic acid |
| 14 | 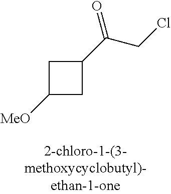 | 3-methoxycyclobutane-1- carboxylic acid |
| 15 | 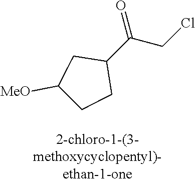 [SMILES: COC1CCC(C(=O)CCl)C1] | 3-methoxycyclopentane-1- carboxylic acid |
| 16 | 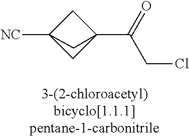 [SMILES: N#CC12CC(C(=O)CCl)(C1)C2] | 3-cyanobicyclo[1.1.1] pentane- 1-carboxylic acid |
| 17 | 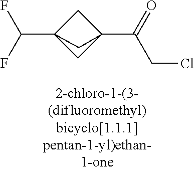 [SMILES: O=C(CCl)C12CC(C(F)F)(C1)C2] | 3-(difluoromethyl) bicyclo[1.1.1] lentane-1-carboxylic acid |
| 18 | 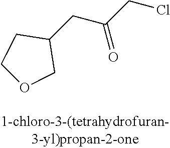 | 2-(oxolan-3-yl)acetic acid |
| 19 | 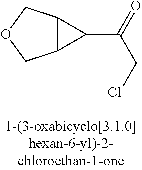 | 3-oxabicyclo[3.1.0]hexane- 6-carboxylic acid |
| 20 | 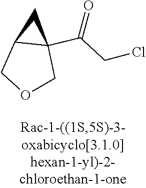 | Rac-(1S,5S)-3- oxabicyclo[3.1.0]hexane-1- carboxylic acid |
| 21 | 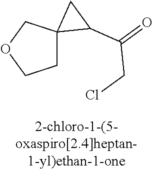 [SMILES: O=C(CCl)C1CC12CCOC2] | 5-oxaspiro[2.4]heptane-1- carboxylic acid |
| 22 | 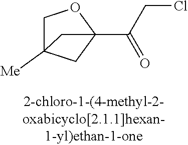 [SMILES: CC12COC(C(=O)CCl)(C1)C2] | 4-methyl-2- oxabicyclo[2.1.1]hexane-1- carboxylic acid |
| 23 | 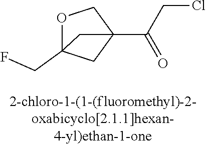 [SMILES: O=C(CCl)C12COC(CF)(C1)C2] | 1-(1-(fluoromethyl)-2- oxabicyclo[2.1.1]hexane-4- carboxylic acid |
| 24 | 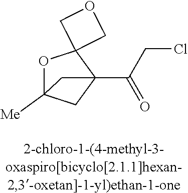 [SMILES: CC12CC(C(=O)CCl)(C1)C1(COC1)O2] | 4-methyl-3- oxaspiro[bicyclo[2.1.1] hexane- 2,3′-oxetane]-1- carboxylic acid |
| 25 | 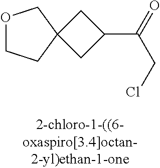 [SMILES: O=C(CCl)C1CC2(CCOC2)C1] | 6-oxaspiro[3.4]octane-2- carboxylic acid |
| 26 | 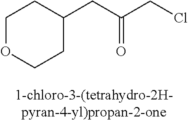 [SMILES: O=C(CCl)CC1CCOCC1] | 2-(oxan-4-yl)acetic acid |
| 27 | 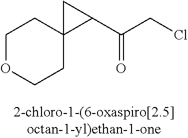 [SMILES: O=C(CCl)C1CC12CCOCC2] | 6-oxaspiro[2.5]octane-1- carboxylic acid |
| 28 | 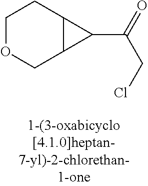 [SMILES: O=C(CCl)C1C2CCOCC21] | 3-oxabicyclo[4.1.0] heptane- 7-carboxylic acid |
| 29 | 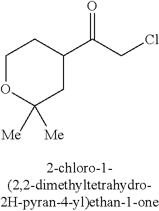 [SMILES: CC1(C)CC(C(=O)CCl)CCO1] | 2,2-dimethyloxane-4- carboxylic acid |
| 30 | 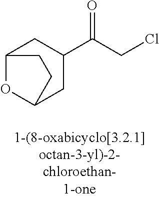 [SMILES: O=C(CCl)C1CC2CCC(C1)O2] | 8-oxabicyclo[3.2.1] octane-3- carboxylic acid |
| 31 | 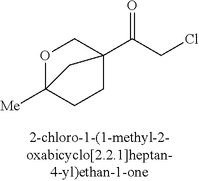 [SMILES: CC12CCC(C(=O)CCl)(CO1)C2] | 1-methyl-2- oxabicyclo[2.2.1] heptane-4- carboxylic acid |
| 32 | 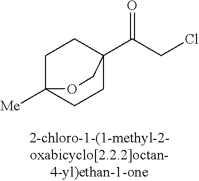 [SMILES: CC12CCC(C(=O)CCl)(CC1)CO2] | 1-methyl-2- oxabicyclo[2.2.2] octane-4- carboxylic acid |
| 33 | 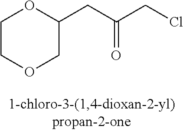 [SMILES: O=C(CCl)CC1COCCO1] | 2-(1,4-dioxan-2-yl)acetic acid |

### Preparation 34: 1-Bromo-3-(tetrahydrofuran-3-yl)propan-2-one

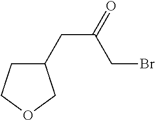

SOCl₂ (1.37 g, 11.52 mmol) was added drop wise to a solution of 2-(tetrahydrofuran-3-yl)acetic acid (1.00 g, 7.68 mmol) in DCM (10.0 mL) at 0° C. and the reaction stirred for 3 h. The mixture was concentrated in vacuo, the crude product dissolved in THF (10.0 mL) the solution cooled to 0° C., TMSCHN₂ (2 M, 7.68 mL, 15.36 mmol) added drop wise and the reaction stirred at 0° C. for 1 h and rt for a further 14 h. The reaction mixture was cooled to 0° C., 48% aq. HBr (2.60 mL, 23.04 mmol) added and the reaction stirred for 1 h. Sat. aq. NaHCO₃ was added to neutralize the solution and the mixture extracted with EtOAc (20 mL×3) and the combined organic layers washed with brine (50 mL) dried over Na₂SO₄ and filtered. The filtrate was concentrated in vacuo to afford 1-bromo-3-(tetrahydrofuran-3-yl)propan-2-one, 850 mg, 53.4% yield.

### Preparation 35: 1-(3-Oxabicyclo[3.1.0]hexan-6-yl)-2-bromoethan-1-one

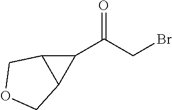

SOCl₂ (779.5 mg, 6.55 mmol) was added dropwise to a mixture of 3-oxabicyclo[3.1.0]hexane-6-carboxylic acid (700 mg, 5.46 mmol) in DCM (15.0 mL) containing one drop of DMF at 0° C. and the reaction stirred for 3 h. The solvent was removed in vacuo and the crude product dissolved in THF (15.0 mL) and the solution cooled to 0° C. TMSCHN₂ (2 M, 5.46 mL) was added dropwise, the reaction stirred at 0° C. for 1 h and at 25° C. for a further 14 h. The reaction mixture was cooled to 0° C., HBr (1.33 g, 48%, 16.38 mmol) added and the mixture stirred for 1 h. The reaction was quenched by the addition of sat. aq. NaHCO₃, then extracted with EtOAc (30 mL×3). The combined organic layers were washed with brine (50 mL), dried over Na₂SO₄ and filtered. The filtrate was evaporated under reduced pressure to afford 1-(3-oxabicyclo[3.1.0]hexan-6-yl)-2-bromoethan-1-one, 750.5 mg, 67.0% yield. ¹H NMR (500 MHz, MeOH-d₄) δ: 2.19-2.21 (m, 1H), 2.28 (d, 2H), 3.77 (d, 2H), 3.96 (d, 2H), 3.99 (s, 2H)

### Preparation 36: 2-Bromo-1-(1-methyl-2-oxabicyclo[2.1.1]hexan-4-yl)ethan-1-one

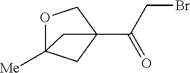 [SMILES: CC12CC(C(=O)CBr)(CO1)C2]

Oxalyl chloride (1.19 mL, 14.06 mmol) was added to 1-methyl-2-oxabicyclo[2.1.1]hexane-4-carboxylic acid (1.00 g, 7.03 mmol) in DCM (12.0 mL) at 0° C., and the reaction stirred at rt for 18 h. The solution was evaporated under reduced pressure to provide 1-methyl-2-oxabicyclo[2.1.1]hexane-4-carbonyl chloride.

TMSCHN₂ (2 M, 7.74 mL) was added to a solution of 1-methyl-2-oxabicyclo[2.1.1]hexane-4-carbonyl chloride (2.26 g, 14.07 mmol) in THF (12 mL) at 0° C. and the reaction stirred for 1.5 h at 0° C. HBr (4.78 mL, 48%, 42.21 mmol) was added drop wise and the reaction stirred for a further 1.5 h. The reaction was diluted with EtOAc and basified with aq. sat. NaHCO₃ to pH 9, and the layers separated. The aqueous phase was extracted with EtOAc (×3), the combined organic extracts were washed with brine, dried over MgSO₄, filtered and evaporated under reduced pressure to afford 2-bromo-1-(1-methyl-2-oxabicyclo[2.1.1]hexan-4-yl)ethan-1-one. ¹H NMR (500 MHz, CDCl₃) δ: 1.47 (s, 3H), 1.93 (d, 2H), 2.02 (d, 2H), 3.98-4.00 (m, 4H).

Alternative Synthesis

Part A: CDI (20.53 g, 126.6 mmol) was added portion wise to a solution of 1-methyl-2-oxabicyclo[2.1.1]hexane-4-carboxylic acid (15 g, 105.5 mmol) in DCM (300 mL) and the mixture was stirred for 5 h at rt. N-methoxymethanamine hydrochloride (10.19 g, 105.5 mmol) was added and the resulting mixture was stirred at rt overnight. The reaction was poured into a mixture of water and ice and extracted with DCM (2×100 mL). The combined organics were washed with brine, dried (Na₂SO₄) and evaporated to dryness under reduced pressure to give N-methoxy-N,1-dimethyl-2-oxabicyclo[2.1.1]hexane-4-carboxamide as a yellow oil (18.2 g). LCMS m/z=186.2 [M+H]⁺

Part B: A solution N-methoxy-N,1-dimethyl-2-oxabicyclo[2.1.1]hexane-4-carboxamide (18.20 g, 98.26 mmol) in Et₂O (150 mL) was cooled to −15° C. and 1.6 M MeLi in Et₂O (19.82 mL, 98.26 mmol) added dropwise. The reaction mixture was warmed to 0° C. for 1.5 h and then warmed to rt. The reaction was quenched with sat. aq. NH₄C1 and extracted with Et₂O (2×). The combined organics were washed with brine, dried (Na₂SO₄) and evaporated to dryness in vacuo to afford 1-(1-methyl-2-oxabicyclo[2.1.1]hexan-4-yl)ethan-1-one as a yellow oil (13.5 g, 98%) which was used without further purification.

Part C: A solution of 1-(1-methyl-2-oxabicyclo[2.1.1]hexan-4-yl)ethan-1-one (13.50 g, 96.30 mmol) in DCM (90 mL) and MeOH (15 mL) was cooled at 0° C. and a solution of Br₂ (15.39 g, 96.30 mmol) in DCM (25 mL) was added dropwise and the reaction was stirred from 0 to 15° C. in about 2 h. The reaction was washed (NaHCO₃×2) and extracted with DCM (2×50 mL). The combined organics were dried (Na₂SO₄) and evaporated at 30° C. to afford 2-bromo-1-(1-methyl-2-oxabicyclo[2.1.1]hexan-4-yl)ethan-1-one (19.50 g, crude) as a yellow oil.

### Preparation 37: 2-Bromo-1-(3-fluorobicyclo[1.1.1]pentan-1-yl)ethan-1-one

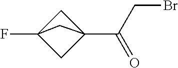

Oxalyl chloride (455.2 μL, 5.38 mmol) was added to 3-fluorobicyclo[1.1.1]pentane-1-carboxylic acid (350.0 mg, 2.69 mmol) in DCM (6.73 mL) at 0° C., the solution stirred for 18 h, then concentrated in vacuo. The residue was suspended in THF (6.73 mL), cooled to 0° C., TMSCHN₂ (2 M, 1.61 mL) added, and the mixture stirred for 1.5 h. HBr (912.8 μL, 8.07 mmol, 48% purity) was added and the reaction stirred for 1 h at 0° C. EtOAc was added to quench the reaction, then aq. sat. NaHCO₃ added until bubbling stopped. The mixture was extracted with EtOAc (×3), the combined organic extracts washed with brine, dried over MgSO₄, filtered, and evaporated under reduced pressure to afford the 2-bromo-1-(3-fluorobicyclo[1.1.1]pentan-1-yl)ethan-1-one, 150.0 mg, 26.9% yield. ¹H NMR (500 MHz, CDCl₃) δ: 1.98-2.20 (m, 6H) 3.83 (s, 2H)

### Preparation 38: 2-Bromo-1-(3-methoxybicyclo[1.1.1]pentan-1-yl)ethan-1-one

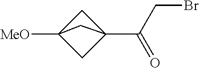 [SMILES: COC12CC(C(=O)CBr)(C1)C2]

Oxalyl chloride (595.3 μL, 7.04 mmol) was added to 3-methoxybicyclo[1.1.1]pentane-1-carboxylic acid (500.4 mg, 3.52 mmol) in DCM (5.87 mL) and the reaction stirred at rt for 18 h. The solution was concentrated in vacuo, the residue suspended in THF (5.83 mL), TMSCHN₂ (439.8 mg, 3.85 mmol) added and the solution stirred for 1 h. HBr (1.19 mL, 48% purity, 10.5 mmol) was added and the reaction stirred at rt for 24 h. The reaction mixture was evaporated under reduced pressure to afford 2-bromo-1-(3-methoxybicyclo[1.1.1]pentan-1-yl)ethan-1-one. ¹H NMR (500 MHz, CDCl₃) δ: 2.21-2.25 (m, 6H), 3.80 (s, 3H), 4.03 (s, 2H).

### Preparations 39 to 42

To a solution of the appropriate amine (1 equiv.) in EtOH was added NaHCO₃(2.0-3.0 equiv.) and the appropriate bromo or chloro ketone (1.1-2.0 equiv.) and the reaction stirred at 80° C. for 14 h. The cooled mixture was concentrated in vacuo and the residue was purified by column chromatography on silica gel eluting with DCM/EtOAc at an appropriate gradient to afford the desired compound.

| Prep. | | |
|---|---|---|
| no | Product, Name, Starting Materials | Yield, Data |
| 39 | 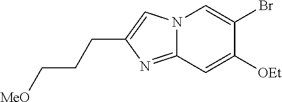 [SMILES: CCOc1cc2nc(CCCOC)cn2cc1Br] | 320.0 mg, 44.3%, as a yellow solid. LCMS m/z = 314.8 [M + H]⁺ ¹H NMR (500 MHz, CDCl₃) δ: 1.53 (t, 3H), 2.02-2.06 (m, 2H), 2.83 (t, 2H), 3.35 (s, 3H), 3.46 (t, 2H), 4.13-4.18 (m, 2H), 7.04 (s, 1H), 7.17 (s, 1H), 8.20 |
| | 6-bromo-7-ethoxy-2-(3- | (s, 1H). |
| | methoxypropyl)imidazo[1,2- | |
| | a]pyridine SM: 4-methoxybutanoyl | |
| | chloride (Chemical Science 2013, | |
| | 4(11), 4187) and 5-bromo-4- | |
| | ethoxypyridin-2-amine | |
| 40^{A} | 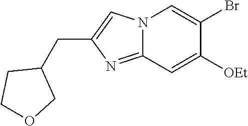 [SMILES: CCOc1cc2nc(CC3CCOC3)cn2cc1Br] | 300 mg, 36% yield, as a yellow oil. LCMS m/z = 327.0 [M + H]⁺ ¹H NMR: (500 MHz, CDCl₃) δ: 1.40-1.50 (m, 3H), 1.60-1.70 (m, 1H), 2.00-2.10 (m, 1H), 2.60-2.70 (m, 1H), 2.75-2.80 (m, 2H), 3.50-3.55 (m, 1H), 3.70-3.80 (m, 1H), 3.80-3.90 (m, 2H), 4.10-4.20 (m, 2H), 6.83 (s, 1H), 7.16 (s, |
| | 6-bromo-7-ethoxy-2- | 1H), 8.16 (s, 1H). |
| | ((tetrahydrofuran-3- | |
| | yl)methyl)imidazo[1,2-a]pyridine | |
| | SM: 1-bromo-3-(tetrahydrofuran-3- | |
| | yl)propan-2-one (Preparation 34) and | |
| | 5-bromo-4-ethoxypyridin-2-amine | |
| 41 | 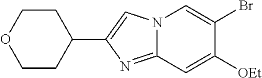 [SMILES: CCOc1cc2nc(C3CCOCC3)cn2cc1Br] | 370 mg, 49.6% yield as a yellow solid. LCMS m/z = 326.8 [M + H]⁺ ¹H NMR (500 MHz, CDCl₃) δ: 1.53 (t, 3H), 1.77-1.87 (m, 2H), 2.03-2.07 (m, 2H), 2.95-3.05 (m, 1H), 3.54-3.60 (m, |
| | 6-bromo-7-ethoxy-2-((tetrahydro- | 2H), 4.06-4.09 (m, 2H), 4.12-4.16 (m, |
| | 2H-pyran-4-yl)imidazo[1,2- | 2H), 6.95 (s, 1H), 7.14 (s, 1H), 8.20 (s, |
| | a]pyridine SM: 2-bromo-1- | 1H). |
| | (tetrahydro-2H-pyran-4-yl)ethan-1- | |
| | one and 5-bromo-4-ethoxypyridin-2- | |
| | amine | |
| 42 | 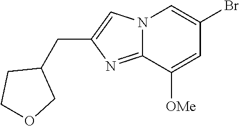 [SMILES: COc1cc(Br)cn2cc(CC3CCOC3)nc12] | 150 mg, 48.9% yield as brown oil. LCMS m/z = 312.8 [M + H]⁺ ¹H NMR (500 MHz, CDCl₃) δ: 1.64- 1.68 (m, 1H), 2.03-2.07 (m, 1H), 2.76- 2.84 (m, 3H), 3.48-3.52 (m, 1H), 3.75- 3.78 (m, 1H), 3.85-3.91 (m, 2H), 4.00 (s, 3H), 6.52 (d, 1H), 7.28 (s, 1H), 7.85 (d, |
| | 6-bromo-8-methoxy-2- | 1H). |
| | ((tetrahydrofuran-3- | |
| | yl)methyl)imidazo[1,2-a]pyridine | |
| | SM: 1-bromo-3-(tetrahydrofuran-3- | |
| | yl)propan-2-one (Preparation 34) and | |
| | 5-bromo-3-methoxypyridin-2-amine | |
| 43^{B} | 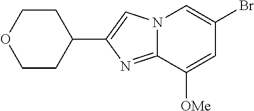 [SMILES: COc1cc(Br)cn2cc(C3CCOCC3)nc12] | 1.80 g, 58.7% yield LCMS m/z = 313.0 [M + H]⁺ |
| | 6-bromo-8-methoxy-2-(tetrahydro- | |
| | 2H-pyran-4-yl)imidazo[1,2- | |
| | a]pyridine SM: 5-bromo-3- | |
| | methoxypyridin-2-amine and 2- | |
| | bromo-1-(tetrahydro-2H-pyran-4- | |
| | yl)ethan-1-one | |

^{A}the reaction mixture was filtered, the filtrate concentrated in vacuo and the residue purified by formic acid modified reverse-phase HPLC.
^{B}EtOAc/EtOH (3:1)/heptane was used as the dry loaded silica gel column solvent

### Preparation 44: 6-Bromo-8-methoxy-2-(tetrahydro-2H-pyran-4-yl)imidazo[1,2-a]pyrazine

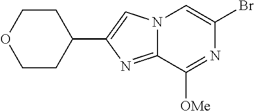 [SMILES: COc1nc(Br)cn2cc(C3CCOCC3)nc12]

A mixture of 5-bromo-3-methoxypyrazin-2-amine (1.0 g, 4.90 mmol), 2-bromo-1-(tetrahydro-2H-pyran-4-yl)ethan-1-one (1.01 g, 4.90 mmol) and NaHCO₃(1.23 g, 14.70 mmol) in EtOH (12 mL) was heated at 80° C. for 18 h. The cooled mixture was filtered through Celite® and the filtrate was concentrated in vacuo. The crude material was purified by column chromatography on silica gel using an Isco autopurification system eluting with EtOAc/heptane (0/100 to 100/0) to afford 6-bromo-8-methoxy-2-(tetrahydro-2H-pyran-4-yl)imidazo[1,2-a]pyrazine, 866 mg, 56.6%, as a white solid. LCMS m/z=311.9 [M+H]^{+ 1}H NMR (400 MHz, CDCl₃) δ: 1.86-1.92 (m, 2H), 2.00-2.05 (m, 2H), 3.23-3.32 (m, 1H), 3.57-3.63 (m, 2H), 4.04-4.09 (m, 2H), 4.29 (s, 3H), 8.14 (s, 1H), 8.61 (s, 1H).

### Preparation 45: 6-Bromo-7-methoxy-2-(tetrahydro-2H-pyran-4-yl)imidazo[1,2-a]pyridine

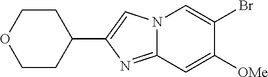 [SMILES: COc1cc2nc(C3CCOCC3)cn2cc1Br]

5-Bromo-4-methoxypyridin-2-amine (40.0 g, 197 mmol) and NaHCO₃(49.7 g, 591 mmol) were added to a solution of 2-bromo-1-(tetrahydro-2H-pyran-4-yl)ethan-1-one (44.9 g, 217 mmol) in EtOH (600 mL) and the reaction heated at reflux for 18 h under Ar(g). The cooled mixture was filtered and the filtrate evaporated under reduced pressure. The crude product was triturated with cold water (600 mL) for 2 h, the solid filtered off and dried to afford the 6-bromo-7-methoxy-2-(tetrahydro-2H-pyran-4-yl)imidazo[1,2-a]pyridine, 54.5 g, 76.5% yield, as light-yellow crystals. LCMS m/z=313.0 [M+H]⁺

### Preparation 46: 6-Bromo-2-(tetrahydro-2H-pyran-4-yl)imidazo[1,2-a]pyrazine

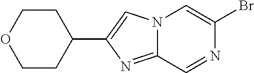 [SMILES: Brc1cn2cc(C3CCOCC3)nc2cn1]

To a solution of 5-bromopyrazine-2-amine (200 mg, 1.15 mmol) in tBuOH (10 mL) was added 2-bromo-1-(tetrahydro-2H-pyran-4-yl)ethan-1-one (952 mg, 4.60 mmol) and NaHCO₃(290 mg, 3.45 mmol) and the reaction stirred at 100° C. for 12 h. The cooled mixture was concentrated in vacuo and the residue purified by prep-HPLC using a Phenomenex Synergi C18 150×30 mm×4 um column, eluting with 16% to 36% of water (0.05% HCl-MeCN) to afford the 6-bromo-2-(tetrahydro-2H-pyran-4-yl)imidazo[1,2-a]pyrazine, 180 mg, 54.34% yield, as a yellow solid. LCMS m/z=282.0 [M+H]^{+ 1}H NMR (500 MHz, DMSO-d₆) δ: 1.71-1.79 (m, 2H), 1.95 (d, 2H), 3.13 (s, 1H), 3.48 (td, 2H), 3.95 (dd, 2H), 8.07 (d, 1H), 8.98-9.04 (m, 2H).

### Preparation 47: 2-(3-Oxabicyclo[3.1.0]hexan-6-yl)-6-bromoimidazo[1,2-a]pyrazine

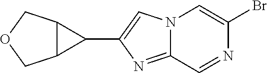 [SMILES: Brc1cn2cc(C3C4COCC43)nc2cn1]

was obtained as a yellow oil in 41.8% yield, 100 mg, from 5-bromopyrazine-2-amine and 1-(3-oxabicyclo[3.1.0]hexan-6-yl)-2-bromoethan-1-one (Preparation 35), following the procedure described in Preparation 46. LCMS m/z=279.9 [M+H]⁺

### Preparation 48: 6-Bromo-8-methoxy-2-((tetrahydrofuran-3-yl)methyl)imidazo[1,2-a]pyrazine

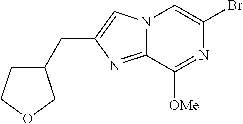 [SMILES: COc1nc(Br)cn2cc(CC3CCOC3)nc12]

NaHCO₃ (494.1 mg, 5.88 mmol) was added to a solution of 5-bromo-3-methoxypyrazin-2-amine (400.0 mg, 1.96 mmol) and 1-bromo-3-(tetrahydrofuran-3-yl)propan-2-one (Preparation 34, 811.7 mg, 3.92 mmol) in tBuOH (20 mL) and the reaction stirred at 100° C. for 72 h. The cooled mixture was concentrated in vacuo and the residue purified by column chromatography on silica gel eluting with MeOH/DCM=1/50 to 1/10. The crude product was purified by prep-HPLC on a Phenomenex Synergi C18 150×30 mm×4 um column, eluting with 22% to 42% of water (0.05% HCl-MeCN) to afford 6-bromo-8-methoxy-2-((tetrahydrofuran-3-yl)methyl)imidazo[1,2-a]pyrazine (70.0 mg, 11.4% yield) as a yellow solid. LCMS m/z=311.9 [M+H]^{+ 1}H NMR (500 MHz, MeOH-d₄) δ: 1.69-1.71 (m, 2H), 2.12-2.16 (m, 1H), 2.68-2.70 (m, 1H), 2.99-3.01 (m, 2H), 3.52-3.54 (m, 1H), 3.79-3.87 (m, 1H), 3.88-3.92 (m, 1H), 4.28 (s, 3H), 8.13 (s, 1H), 8.60 (s, 1H).

### Preparations 49 to 53

To a solution of methyl 6-amino-4-ethoxynicotinate (Preparation 3) (1.0 equiv.) in EtOH was added NaHCO₃ (2.0-3.0 equiv.), the appropriate bromo or chloro ketone (1.0 equiv.) and KI (0.1 equiv.) and the reaction stirred at 80° C. for 14 h. The cooled mixture was filtered and the filtrate concentrated in vacuo. The residue was purified by prep-TLC eluting with DCM/MeOH at an appropriate gradient to afford the title compound.

| Prep. No | Structure and name | Starting materials, Yield and Data |
|---|---|---|
| 49^{A} | 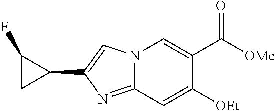 [SMILES: CCOc1cc2nc([C@H]3C[C@H]3F)cn2cc1C(=O)OC] | 2-chloro-1-((1R,2R)-2- fluorocyclopropyl)ethan-1-one (Preparation 10) 70 mg, 49% yield as a white solid. LCMS m/z = 279.1 [M + H]⁺ |
| | methyl 7-ethoxy-2-((1R,2R)-2- | |
| | fluorocyclopropyl)imidazo[1,2- | |
| | a]pyridine-6-carboxylate | |
| 50 | 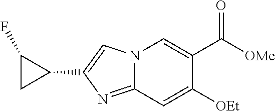 [SMILES: CCOc1cc2nc([C@@H]3C[C@@H]3F)cn2cc1C(=O)OC] | 2-chloro-1-((1S,2S)-2- fluorocyclopropyl)ethan-1-one (Preparation 11) 60 mg, 36.8% yield as a white solid LCMS m/z = 279.0 [M + H]⁺ |
| | methyl 7-ethoxy-2-((1S,2S)-2- | |
| | fluorocyclopropyl)imidazo[1,2- | |
| | a]pyridine-6-carboxylate | |
| 51 | 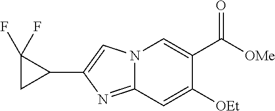 [SMILES: CCOc1cc2nc(C3CC3(F)F)cn2cc1C(=O)OC] | 2-chloro-1-(2,2- difluorocyclopropyl)ethan-1-one (Preparation 5) 50.0 mg, 31.8% yield, as a yellow solid. LCMS m/z = 297.1 [M + H]⁺ |
| | methyl 2-(2,2-difluorocyclopropyl)- | |
| | 7-ethoxyimidazo[1,2-a]pyridine-6- | |
| | carboxylate | |
| 52 | 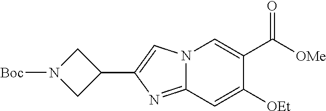 [SMILES: CCOc1cc2nc(C3CN(C(=O)OC(C)(C)C)C3)cn2cc1C(=O)OC] | tert-butyl 3-(2-chloroacetyl)azetidine- 1-carboxylate 40 mg, 66.9% yield, as a yellow solid. ¹H NMR (400 MHz, CDCl₃) δ: 1.40-1.50 (m, 9H), 1.53 (t, 3H), 3.92 (s, 3H), 4.10-4.16 (m, 4H), 4.31 (t, 2H), 4.35-4.40 (m, 1H), 6.87 (d, 1H), 7.33 (s, 1H), 8.62 (d, 1H) |
| | Methyl 2-(1-(tert- | |
| | butoxycarbonyl)azetidin-3-yl)-7- | |
| | ethoxyimidazolo[1,2-a]pyridine-6- | |
| | carboxylate | |
| 53 | 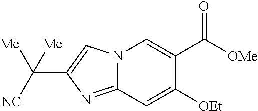 [SMILES: CCOc1cc2nc(C(C)(C)C#N)cn2cc1C(=O)OC] | 4-chloro-2,2-dimethyl-3- oxobutanenitrile (Preparation 9) 50.0 mg, 83.6% yield, as a white solid. LCMS m/z = 288.1 [M + H]⁺ ¹H NMR (400 MHz, CDCl₃) δ: 1.52 (t, 3H), 1.81 (s, 6H), 3.93 (s, 3H), 4.17-4.10 (m, 2H), 6.90 (s, 1H), 7.49 |
| | Methyl 2-(2-cyanopropan-2-yl)-7- | (s, 1H), 8.63 (s, 1H) |
| | ethoxyimidazo[1,2-a]pyridine-6- | |
| | carboxylate | |

^{A}only 0.7 equiv. amine was used in the reaction
^{B}2.0 equiv. of amine was used in the reaction

### Preparation 54: Rac-methyl 7-ethoxy-2-((1S,2R)-2-fluorocyclopropyl)imidazo[1,2-a]pyridine-6-carboxylate

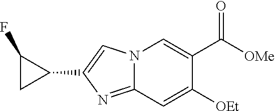 [SMILES: CCOc1cc2nc([C@@H]3C[C@H]3F)cn2cc1C(=O)OC]

To a solution of Rac-2-chloro-1-((1S,2R)-2-fluorocyclopropyl)ethan-1-one (Preparation 12, 90.0 mg, 0.66 mmol) in EtOH (1 mL) was added NaHCO₃ (110.7 mg, 1.32 mmol), methyl 6-amino-4-ethoxynicotinate (Preparation 3, 103.5 mg, 0.53 mmol) and KI (10.9 mg, 0.07 mmol) and the reaction stirred at 80° C. for 14 h. The cooled reaction was filtered and the filtrate concentrated in vacuo. The crude product was purified by column chromatography on silica gel using Combiflash®, eluting with DCM/EtOAc (50/50) to afford rac-methyl 7-ethoxy-2-((1S,2R)-2-fluorocyclopropyl)imidazo[1,2-a]pyridine-6-carboxylate in 41.4% yield, as a white solid. LCMS m/z=279.0 [M+H]⁺

### Preparation 55: Methyl 2-(difluoromethyl)-7-ethoxyimidazo[1,2-a]pyridine-6-carboxylate

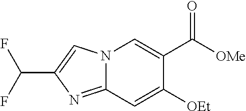 [SMILES: CCOc1cc2nc(C(F)F)cn2cc1C(=O)OC]

A solution of methyl 6-amino-4-ethoxynicotinate (Preparation 3, 500 mg, 2.55 mmol) and 3-bromo-1,1-difluoropropan-2-one (756 mg, 3.06 mmol) in EtOH (20 mL) was heated at reflux for 96 h. The cooled mixture was concentrated in vacuo, the residue suspended in water (10 mL) and NaHCO₃ (428 mg, 5.10 mmol) added. The solution was extracted with CHCl₃ (3×10 mL), the combined organic phases dried over Na₂SO₄, filtered and evaporated under reduced pressure, to afford methyl 2-(difluoromethyl)-7-ethoxyimidazo[1,2-a]pyridine-6-carboxylate, 640 mg, as a brown viscous oil. LCMS m/z=271.2 [M+H]^{+ 1}H NMR (400 MHz, CDCl₃): δ 1.49 (t, 3H), 3.90 (s, 3H), 4.12 (q, 2H), 6.57-6.98 (m, 2H), 7.65 (s, 1H), 8.64 (s, 1H).

### Preparation 56: Methyl 2-(difluoromethyl)-7-isopropoxyimidazo[1,2-a]pyridine-6-carboxylate

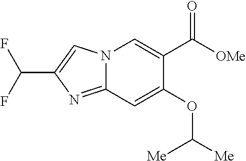 [SMILES: COC(=O)c1cn2cc(C(F)F)nc2cc1OC(C)C]

A mixture of methyl 6-amino-4-isopropoxynicotinate (Preparation 2, 1.0 g, 4.76 mmol), 3-bromo-1,1-difluoro-propan-2-one (1.65 g, 9.52 mmol) and NaHCO₃ (800 mg, 9.52 mmol) in EtOH (20 mL) was heated at 80° C. for 16 h. The cooled mixture was diluted with H₂O (25 mL) and extracted with DCM (3×50 mL). The combined organic layers were washed with brine, dried over Na₂SO₄, filtered and evaporated under reduced pressure to afford methyl 2-(difluoromethyl)-7-isopropoxyimidazo[1,2-a]pyridine-6-carboxylate as a brown solid, 1.20 g, 88.9% yield. LCMS m/z=285.2 [M+H]⁺

### Preparation 57: Methyl 2-(1,1-difluoroethyl)-7-isopropoxyimidazo[1,2-a]pyridine-6-carboxylate

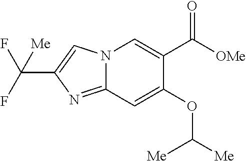 [SMILES: COC(=O)c1cn2cc(C(C)(F)F)nc2cc1OC(C)C]

was obtained as a brown solid, 1.2 g, 84.5%, from 1-chloro-3,3-difluorobutan-2-one (Preparation 4) and methyl 6-amino-4-isopropoxynicotinate (Preparation 2), following the procedure described in Preparation 56. LCMS m/z=299.0 [M+H]⁺

### Preparation 58: Methyl 8-hydroxy-2-(1-methoxycyclopropyl)imidazo[1,2-a]pyridine-6-carboxylate

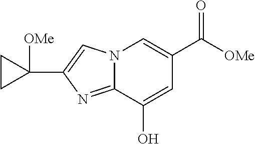 [SMILES: COC(=O)c1cc(O)c2nc(C3(OC)CC3)cn2c1]

A mixture of methyl 6-amino-5-hydroxynicotinate (310 mg, 1.84 mmol), 2-chloro-1-(1-methoxycyclopropyl)ethan-1-one (Preparation 13, 301 mg, 2.02 mmol) and LiBr (159.8 mg, 1.84 mmol) in EtOH (7 mL) was heated at reflux for 48 h. The cooled mixture was evaporated under reduced pressure, the residue dissolved in EtOAc (20 mL) and stirred with a solution of NaHCO₃ (195 mg, 1.84 mmol) in water (3 mL) for 1 h. The layers were separated and the organic phase evaporated under reduced pressure to afford methyl 8-hydroxy-2-(1-methoxycyclopropyl)imidazo[1,2-a]pyridine-6-carboxylate, 610.0 mg. LCMS m/z=263.0 [M+H]⁺

### Preparation 59: Methyl 7-isopropoxy-2-(tetrahydro-2H-pyran-4-yl)imidazo[1,2-a]pyridine-6-carboxylate

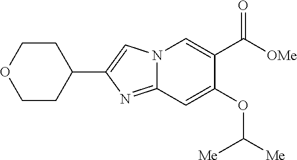 [SMILES: COC(=O)c1cn2cc(C3CCOCC3)nc2cc1OC(C)C]

A mixture of methyl 6-amino-4-isopropoxynicotinate (500 mg, 2.38 mmol) and 2-chloro-1-(tetrahydro-2H-pyran-4-yl)ethan-1-one (1.05 g, 6.10 mmol) in EtOH (10 mL) was heated at 90° C. for 48 h. The cooled mixture was diluted with sat. aq. NaHCO₃ (20 mL) and extracted with EtOAc (3×20 mL). The combined organic layers were dried over Na₂SO₄ and concentrated in vacuo and the crude product purified by HPLC to afford methyl 7-isopropoxy-2-(tetrahydro-2H-pyran-4-yl)imidazo[1,2-a]pyridine-6-carboxylate, 60 mg, 7.9% yield. LCMS m/z=319.2 [M+H]⁺

### Preparation 60: Methyl 7-isopropoxy-2-(1-methyl-2-oxabicyclo[2.1.1]hexan-4-yl)imidazo[1,2-a]pyridine-6-carboxylate

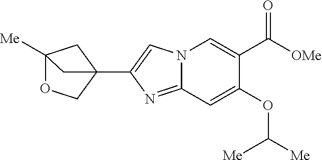 [SMILES: COC(=O)c1cn2cc(C34COC(C)(C3)C4)nc2cc1OC(C)C]

A mixture of methyl 6-amino-4-isopropoxynicotinate (Preparation 2, 450 mg, 2.14 mmol) and 2-chloro-1-(1-methyl-2-oxabicyclo[2.1.1]hexan-4-yl)ethan-1-one (Preparation 6, 374 mg, 2.14 mmol) in EtOH (10 mL) was heated at reflux for 48 h. The cooled mixture was diluted with water (5 mL), washed with EtOAc (5 mL), dried over Na₂SO₄ and concentrated in vacuo. The crude product was purified by HPLC to afford methyl 7-isopropoxy-2-(1-methyl-2-oxabicyclo[2.1.1]hexan-4-yl)imidazo[1,2-a]pyridine-6-carboxylate, 161 mg, 22.7% yield as a dark red solid. LCMS m/z=331.2 [M+H]⁺

### Preparation 61: Methyl 7-isopropoxy-2-(3-methoxypropyl)imidazo[1,2-a]pyridine-6-carboxylate

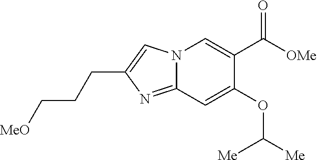 [SMILES: COCCCc1cn2cc(C(=O)OC)c(OC(C)C)cc2n1]

was obtained 72.8 mg, 9.87% yield, from methyl 6-amino-4-isopropoxynicotinate (Preparation 2) and 4-methoxybutanoyl chloride (Chemical Science 2013, 4(11), 4187) following the procedure described in Preparation 60. LCMS m/z=307.2 [M+H]⁺

### Preparation 62: Methyl 8-methoxy-2-(1-methoxycyclopropyl)imidazo[1,2-a]pyridine-6-carboxylate

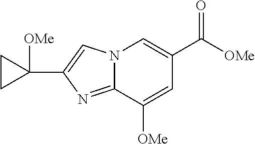 [SMILES: COC(=O)c1cc(OC)c2nc(C3(OC)CC3)cn2c1]

A solution of diazomethane in MTBE (7.30 mL, 6.06 mmol, 0.83 M) was added to a solution of methyl 8-hydroxy-2-(1-methoxycyclopropyl)imidazo[1,2-a]pyridine-6-carboxylate (Preparation 58, 530 mg, 2.02 mmol) in benzene (10 mL), and the reaction stirred at rt for 18 h. Acetic acid was added and the mixture concentrated in vacuo. The crude product was purified by column chromatography on silica gel to afford methyl 8-methoxy-2-(1-methoxycyclopropyl)imidazo[1,2-a]pyridine-6-carboxylate, 150 mg, 21.5%. LCMS m/z=277.2 [M+H]⁺

### Preparations 63 to 71

To a solution of the appropriate halide (1.0 equiv.) in MeOH was added TEA (10.0 equiv.) and Pd(dppf)Cl₂ (0.2 equiv.) at 15° C. under N₂. The mixture was stirred at 80° C. under CO at 50 psi for 24 h. The cooled reaction was filtered through Celite® and the filtrate concentrated in vacuo. The residue was purified by column chromatography on silica gel using Combiflash® eluting with DCM/EtOAc or PE/EtOAc, at an appropriate gradient to afford the title compound.

| Prep. No | Structure, Name, Starting Materials | Yield, Data |
|---|---|---|
| 63 | 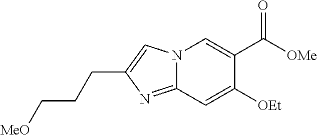 [SMILES: CCOc1cc2nc(CCCOC)cn2cc1C(=O)OC] | 170.0 mg, 60.7% yield as a yellow solid. LCMS m/z = 293.0 [M + H]⁺ ¹H NMR: (500 MHz, CDCl₃) δ: 1.51 (t, 3H), 2.01-2.05 (m, 2H), 2.81 (t, 2H), 3.36 (s, 3H), 3.46 (t, 2H), 3.91 (s, 3H), 4.10-4.15 (m, 2H), 6.84 (s, 1H), 7.22 (s, 1H), 8.62 (s, 1H) |
| | Methyl 7-ethoxy-2-(3- | |
| | methoxypropyl)imidazo[1,2- | |
| | a]pyridine-6-carboxylate | |
| | SM: 6-bromo-7-ethoxy-2-(3- | |
| | methoxypropyl)imidazo[1,2- | |
| | a]pyridine (Preparation 39) | |
| 64^{A} | 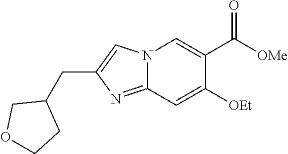 [SMILES: CCOc1cc2nc(CC3CCOC3)cn2cc1C(=O)OC] | 170 mg, 61% yield, as brown oil LCMS m/z = 305.0 [M + H]⁺ ¹H NMR (500 MHz, CDCl₃) δ: 1.51 (t, 3H), 1.60-1.70 (m, 1H), 2.10-2.20 (m, 1H), 2.65-2.75 (m, 1H), 2.75-2.80 (m, 2H), 3.50-3.55 (m, 1H), 3.70-3.80 (m, 1H), 3.90- 3.95 (m, 5H), 4.12 (q, 2H), 6.83 (s, 1H), 7.22 (s, 1H), 8.62 (s, 1H) |
| | Methyl 7-ethoxy-2- | |
| | ((tetrahydrofuran-3- | |
| | yl)methyl)imidazo[1,2-a]pyridine- | |
| | 6-carboxylate SM: 6-bromo-7- | |
| | ethoxy-2-((tetrahydrofuran-3- | |
| | yl)methyl)imidazo[1,2-a]pyridine | |
| | (Preparation 40) | |
| 65 | 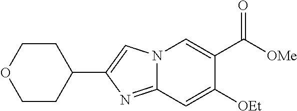 [SMILES: CCOc1cc2nc(C3CCOCC3)cn2cc1C(=O)OC] | 200 mg, 60.9% yield, as a yellow solid. LCMS m/z = 305.1 [M + H]⁺ ¹H NMR: (500 MHz, CDCl₃) δ: 1.52 (t, 3H), 1.82-1.88 (m, 2H), 2.02-2.05 (m, 2H), 2.93-3.03 (m, 1H), 3.55-3.61 (m, 2H), 3.92 (s, 3H), 4.06-4.10 (m, 2H), 4.10-4.15 |
| | Methyl 7-ethoxy-2-(tetrahydro-2H- | (m, 2H), 6.85 (s, 1H), 7.20 (s, 1H), |
| | pyran-4-yl)imidazo[1,2-a]pyridine- | 8.63 (s, 1H) |
| | 6-carboxylate SM: 6-bromo-7- | |
| | ethoxy-2-((tetrahydro-2H-pyran-4- | |
| | yl)imidazo[1,2-a]pyridine | |
| | (Preparation 41) | |
| 66^{B} | 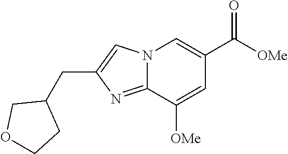 [SMILES: COC(=O)c1cc(OC)c2nc(CC3CCOC3)cn2c1] | 210 mg, 90.0% yield, as a yellow solid. LCMS m/z = 291.1 [M + H]⁺ ¹H NMR: (400 MHz, CDCl₃) δ: 1.67-1.71 (m, 1H), 2.04-2.10 (m, 1H), 2.81-2.88 (m, 3H), 3.52-3.54 (m, 1H), 3.76-3.79 (m, 1H), 3.86- 3.92 (m, 2H), 3.95 (s, 3H), 4.06 (s, 3H), 6.99 (s, 1H), 7.40 (s, 1H), 8.51 (s, 1H) |
| | Methyl 8-methoxy-2- | |
| | ((tetrahydrofuran-3- | |
| | yl)methyl)imidazo[1,2-a]pyridine- | |
| | 6-carboxylate SM: 6-bromo-8- | |
| | methoxy-2-((tetrahydrofuran-3- | |
| | yl)methyl)imidazo[1,2-a]pyridine | |
| | (Preparation 42) | |
| 67^{B} | 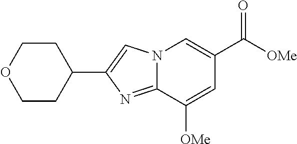 [SMILES: COC(=O)c1cc(OC)c2nc(C3CCOCC3)cn2c1] | 60 mg, 64.3% yield, as a yellow solid LCMS m/z = 291.2 [M + H]⁺ ¹H NMR: (400 MHz, CDCl₃) δ: 1.83-1.87 (m, 2H), 2.05-2.10 (m, 2H), 3.05-3.07 (m, 1H), 3.54-3.59 (m, 2H), 3.95 (s, 3H), 4.05-4.10 (m, 5H), 6.98 (s, 1H), 7.37 (s, 1H), 8.52 (s, 1H) |
| | methyl 8-methoxy-2-(tetrahydro- | |
| | 2H-pyran-4-yl)imidazo[1,2- | |
| | a]pyridine-6-carboxylate SM: 6- | |
| | bromo-8-methoxy-2-(tetrahydro- | |
| | 2H-pyran-4-yl)imidazo[1,2- | |
| | a]pyridine (Preparation 43) | |
| 68^{B} | 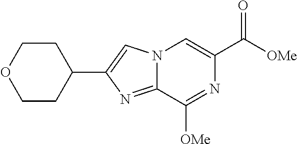 [SMILES: COC(=O)c1cn2cc(C3CCOCC3)nc2c(OC)n1] | 200 mg, 89.3% yield, as a yellow solid. LCMS m/z = 292.3 [M + H]⁺ ¹H NMR: (500 MHz, MeOH-d₄) δ: 1.78-1.81 (m, 2H), 1.83-1.87 (m, 2H), 3.02-3.06 (m, 1H), 3.57-3.62 (m, 2H), 3.96 (s, 3H), 4.02-4.20 (m, 2H), 4.19 (s, 3H), 7.85 (s, 1H), 8.87 (s, 1H) |
| | methyl 8-methoxy-2-(tetrahydro- | |
| | 2H-pyran-4-yl)imidazo[1,2- | |
| | a]pyrazine-6-carboxylate SM: 6- | |
| | bromo-8-methoxy-2-(tetrahydro- | |
| | 2H-pyran-4-yl)imidazo[1,2- | |
| | a]pyrazine (Preparation 44) | |
| 69^{B} | 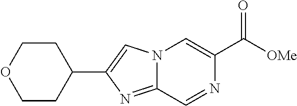 [SMILES: COC(=O)c1cn2cc(C3CCOCC3)nc2cn1] | 110 mg, 66.0% yield, as a brown solid. LCMS m/z = 262.1 [M + H]⁺ |
| | methyl 2-(tetrahydro-2H-pyran-4- | |
| | yl)imidazo[1,2-a]pyrazine-6- | |
| | carboxylate SM: 6-bromo-2- | |
| | (tetrahydro-2H-pyran-4- | |
| | yl)imidazo[1,2-a]pyrazine | |
| | (Preparation 46) | |
| 70 | 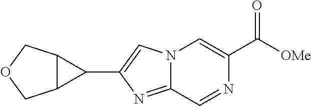 [SMILES: COC(=O)c1cn2cc(C3C4COCC43)nc2cn1] | 70.0 mg 75.6% yield, as a yellow solid. LCMS m/z = 259.9 [M + H]⁺ |
| | methyl 2-(3- | |
| | oxabicyclo[3.1.0]hexan-6- | |
| | yl)imidazo[1,2-a]pyrazine-6- | |
| | carboxylate SM: 2-(3- | |
| | oxabicyclo[3.1.0]hexan-6-yl)-6- | |
| | bromoimidazo[1,2-a]pyrazine | |
| | (Preparation 47) | |
| 71^{B} | 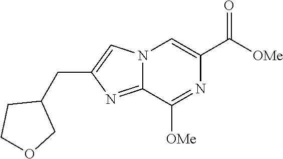 [SMILES: COC(=O)c1cn2cc(CC3CCOC3)nc2c(OC)n1] | Yellow solid, 60 mg, 91.9% LCMS m/z = 292.3 [M + H]⁺ |
| | methyl 8-methoxy-2- | |
| | ((tetrahydrofuran-3- | |
| | yl)methyl)imidazo[1,2-a]pyrazine- | |
| | 6-carboxylate SM: 6-bromo-8- | |
| | methoxy-2-((tetrahydrofuran-3- | |
| | yl)methyl)imidazo[1,2-a]pyrazine | |
| | (Preparation 48) | |

^{A}the crude product was purified by prep-TLC eluting with DCM/MeOH (91/9)
^{B}0.1 equiv. Pd(dppf)Cl₂ used

### Preparation 72: Methyl 7-methoxy-2-(tetrahydro-2H-pyran-4-yl)imidazo[1,2-a]pyridine-6-carboxylate

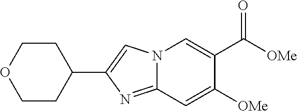 [SMILES: COC(=O)c1cn2cc(C3CCOCC3)nc2cc1OC]

A mixture of 6-bromo-7-methoxy-2-(tetrahydro-2H-pyran-4-yl)imidazo[1,2-a]pyridine (Preparation 45, 54.5 g, 175 mmol), TEA (21.3 g, 210 mmol) and Pd(dppf)Cl₂. DCM (1.43 g, 1.75 mmol) in MeOH (700 mL) were shaken under 40 bar of CO at 130° C. for 16 h. The cooled mixture was filtered and evaporated under reduced pressure. The crude material was taken up in water (250 mL) and extracted with EtOAc (3×200 mL). The combined organic extracts were dried over Na₂SO₄, filtered and concentrated in vacuo. The residue was triturated with a minimum volume of EtOAc, filtered and dried to afford methyl 7-methoxy-2-(tetrahydro-2H-pyran-4-yl)imidazo[1,2-a]pyridine-6-carboxylate, 22.5 g, 44.3% yield, as a pink solid. Additional product was obtained by evaporation of the filtrate, 14 g. 27.6% yield. LCMS m/z=291.0 [M+H]⁺

### Preparation 73: 8-Methoxy-2-(tetrahydro-2H-pyran-4-yl)imidazo[1,2-a]pyrazine-6-carbonitrile

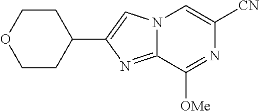 [SMILES: COc1nc(C#N)cn2cc(C3CCOCC3)nc12]

A mixture of 6-bromo-8-methoxy-2-(tetrahydro-2H-pyran-4-yl)imidazo[1,2-a]pyrazine (Preparation 44, 866 mg, 2.77 mmol), Zn(CN)₂ (652 mg, 5.55 mmol), and Pd(PPh₃)₄ (320 mg, 0.277 mmol) in DMF (7.0 mL) was purged with N₂ for 5 min, the reaction vessel sealed and heated at 120° C. for 16 h. The cooled reaction mixture was partitioned between EtOAc and brine and the layers separated. The aqueous solution was extracted with EtOAc (3×15 mL), the combined organic extracts washed with brine, dried over MgSO₄, filtered and the filtrate concentrated in vacuo. The crude product was purified by column chromatography on silica gel using an Isco autopurification system eluting with EtOAc/heptane (0/100 to 100/0) to afford 8-methoxy-2-(tetrahydro-2H-pyran-4-yl)imidazo[1,2-a]pyrazine-6-carbonitrile, 430 mg, 59.8% yield, as a white solid. LCMS m/z=259.1 [M+H]⁺

### Preparation 74 A and 74 B: 8-Methoxy-2-(tetrahydro-2H-pyran-4-yl)imidazo[1,2-a]pyrazine-6-carboxylic acid and 8-hydroxy-2-(tetrahydro-2H-pyran-4-yl)imidazo[1,2-a]pyrazine-6-carboxylic acid

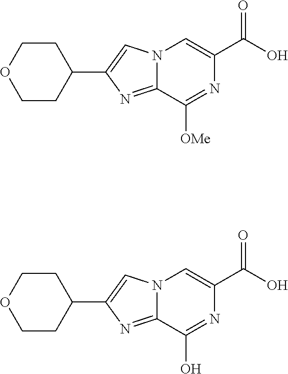 [SMILES: COc1nc(C(=O)O)cn2cc(C3CCOCC3)nc12 ; O=C(O)c1cn2cc(C3CCOCC3)nc2c(O)n1]

A mixture of 8-methoxy-2-(tetrahydro-2H-pyran-4-yl)imidazo[1,2-a]pyrazine-6-carbonitrile (Preparation 73, 430 mg, 1.66 mmol) and NaOH (332 mg, 8.30 mmol) in MeOH (5.0 mL) and water (6.0 mL) was stirred in a sealed vessel at 100° C. for 12 h. The pH of the cooled reaction was adjusted to 2 with aqueous HCl (10 M), and the resulting mixture was filtered. The filtered solid was dried to afford a mixture of 8-methoxy-2-(tetrahydro-2H-pyran-4-yl)imidazo[1,2-a]pyrazine-6-carboxylic acid and 8-hydroxy-2-(tetrahydro-2H-pyran-4-yl)imidazo[1,2-a]pyrazine-6-carboxylic acid, 246 mg as a light yellow solid. LCMS m/z=264.1 [M+H]⁺, 278.1 [M+H]⁺

### Preparation 75: 7-Methoxy-2-(tetrahydro-2H-pyran-4-yl)imidazo[1,2-a]pyridine-6-carboxylic acid

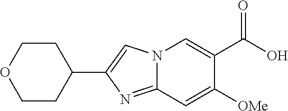 [SMILES: COc1cc2nc(C3CCOCC3)cn2cc1C(=O)O]

A solution of NaOH (8.4 g, 210 mmol) in water (30 mL) was added to a solution of methyl 7-methoxy-2-(tetrahydro-2H-pyran-4-yl)imidazo[1,2-a]pyridine-6-carboxylate (Preparation 72, 30.5 g, 105 mmol) in MeOH (350 mL) and the reaction heated at reflux for 2 h. The cooled reaction mixture was concentrated in vacuo, the residue taken up in water (250 mL) and extracted with MTBE (2×20 mL). The aqueous solution was acidified with 10 N HCl (˜10.5 mL) to pH 5, then concentrated in vacuo to a volume of approx. 70 mL and cooled to 5° C. The resulting solid was filtered off, washed with cold water (3×30 mL) and dried to afford 7-methoxy-2-(tetrahydro-2H-pyran-4-yl)imidazo[1,2-a]pyridine-6-carboxylic acid, 16.8 g, 57.8% yield, as a grey solid. LCMS m/z=277.2 [M+H]⁺

### Preparation 76: 2-(Difluoromethyl)-7-isopropoxyimidazo[1,2-a]pyridine-6-carboxylic acid

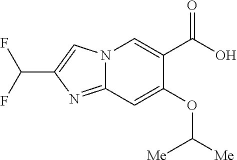 [SMILES: CC(C)Oc1cc2nc(C(F)F)cn2cc1C(=O)O]

A mixture of methyl 2-(difluoromethyl)-7-isopropoxyimidazo[1,2-a]pyridine-6-carboxylate (Preparation 56, 1.20 g, 4.22 mmol) and K₂CO₃ (1.46 g, 10.6 mmol) in H₂O (10.0 mL) and MeOH (3.0 mL) was stirred at rt for 24 h. The mixture was concentrated in vacuo, the residue dissolved in H₂O (15 mL) and acidified using HCl to pH 4-5. The resulting precipitate was filtered off, washed with water and air-dried to provide 2-(difluoromethyl)-7-isopropoxyimidazo[1,2-a]pyridine-6-carboxylic acid as a white solid, 1.00 g, 87.7% yield. LCMS m/z=271.2 [M+H]⁺

### Preparation 77: 2-(1,1-Difluoroethyl)-7-isopropoxyimidazo[1,2-a]pyridine-6-carboxylic acid

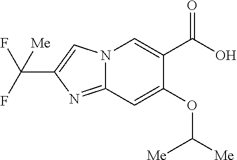 [SMILES: CC(C)Oc1cc2nc(C(C)(F)F)cn2cc1C(=O)O]

was obtained as a white solid, 700 mg, 61.5% yield, from methyl 2-(1,1-difluoroethyl)-7-isopropoxyimidazo[1,2-a]pyridine-6-carboxylate (Preparation 57), following the procedure described in Preparation 76. LCMS m/z=285.2 [M+H]⁺

### Preparation 78: 7-Isopropoxy-2-(1-methyl-2-oxabicyclo[2.1.1]hexan-4-yl)imidazo[1,2-a]pyridine-6-carboxylic acid

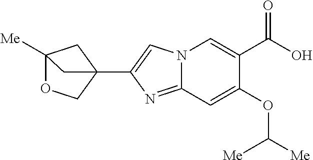 [SMILES: CC(C)Oc1cc2nc(C34COC(C)(C3)C4)cn2cc1C(=O)O]

A solution of methyl 7-isopropoxy-2-(1-methyl-2-oxabicyclo[2.1.1]hexan-4-yl)imidazo[1,2-a]pyridine-6-carboxylate (Preparation 60, 160.5 mg, 0.486 mmol) and NaOH (25.2 mg, 0.632 mmol) in H₂O (2 mL) and MeOH (3 mL) were stirred at rt for 24 h. HCl (10 M, 63.15 μL) was added and the mixture evaporated under reduced pressure to afford 7-isopropoxy-2-(1-methyl-2-oxabicyclo[2.1.1]hexan-4-yl)imidazo[1,2-a]pyridine-6-carboxylic acid, containing NaCl as a white solid. LCMS m/z=317.2 [M+H]⁺

### Preparation 79: 6-Chloro-4-methoxy-N-(pyridin-2-yl)nicotinamide

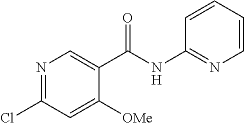 [SMILES: COc1cc(Cl)ncc1C(=O)Nc1ccccn1]

To a mixture of 6-chloro-4-methoxypyridine-3-carboxylic acid (2.0 g, 10.66 mmol), pyridin-2-amine (1.0 g, 10.66 mmol) and DIPEA (6.89 g, 53.30 mmol) in EtOAc (30.0 mL) was added T31³® (20.35 g, 32.0 mmol, 50% solution in EtOAc) and the reaction stirred at rt for 18 h. The mixture was partitioned between EtOAc and H₂O and the layers separated. The organic phase was washed with brine, dried over anhydrous MgSO₄, filtered and the filtrate evaporated in vacuo. The crude product was purified by column chromatography on silica gel using an ISCO autopurification system, eluting with EtOAc/heptane (0/100 to 100/0) to afford 6-chloro-4-methoxy-N-(pyridin-2-yl)nicotinamide, 1.10 g, 39.1% yield, as a yellow solid. LCMS m/z=264.0 [M+H]⁺

### Preparation 80: 6-Chloro-N-(6-ethylpyridin-2-yl)-4-methoxynicotinamide

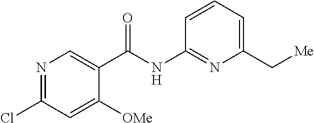 [SMILES: CCc1cccc(NC(=O)c2cnc(Cl)cc2OC)n1]

was obtained as a yellow solid, 1.91 g, 93.5% yield, from 6-chloro-4-methoxynicotinic acid and 6-ethylpyridin-2-amine, following the procedure described in Preparation 79. LCMS m/z=292.0 [M+H]⁺

### Preparation 81: 6-Chloro-4-methoxy-N-(6-methoxypyridin-2-yl)nicotinamide

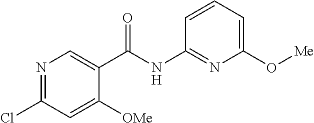 [SMILES: COc1cccc(NC(=O)c2cnc(Cl)cc2OC)n1]

was obtained as a yellow solid, 1.20 g, 58.4% yield, from 6-chloro-4-methoxynicotinic acid and 6-methoxypyridin-2-amine, following the procedure described in Preparation 79. LCMS m/z=294.0 [M+H]⁺

### Preparation 82: 6-Chloro-4-methoxy-N-(6-(trifluoromethyl)pyridin-2-yl)nicotinamide

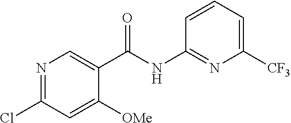 [SMILES: COc1cc(Cl)ncc1C(=O)Nc1cccc(C(F)(F)F)n1]

One drop of DMF was added to a solution of 6-chloro-4-methoxy-pyridine-3-carboxylic acid (375 mg, 2.0 mmol) in THF (6 mL) and the solution cooled to 0° C. (COCl)₂ (170 μL, 2.0 mmol) was slowly added and the reaction stirred for 1 h. TEA (416 μL, 3.0 mmol) and 6-(trifluoromethyl)pyridin-2-amine (324 mg, 2.0 mmol) were added at 0° C. and the reaction stirred at rt for 12 h. The reaction was quenched with sat. aq. NaHCO₃ solution and extracted with EtOAc (15 mL×3). The combined organic layers were dried over anhydrous MgSO₄, filtered and concentrated in vacuo. The residue was purified by column chromatography on silica gel using an Isco purification system eluting with EtOAc/heptane (0/100 to 100/0) to afford 6-chloro-4-methoxy-N-(6-(trifluoromethyl)pyridin-2-yl)nicotinamide (493.0 mg, 74.3% yield). LCMS m/z=332.0 [M+H]⁺

### Preparation 83: 6-Chloro-N-(1-(difluoromethyl)-1H-pyrazol-3-yl)-4-methoxynicotinamide

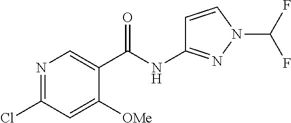 [SMILES: COc1cc(Cl)ncc1C(=O)Nc1ccn(C(F)F)n1]

was obtained as a light yellow solid, 190 mg, 31.3% yield, from 6-chloro-4-methoxynicotinic acid and 1-(difluoromethyl)pyrazol-3-amine, following the procedure described in Preparation 82. LCMS/z=303.0 [M+H]⁺

### Preparation 84: 6-Chloro-N-(6-methoxypyridin-2-yl)nicotinamide

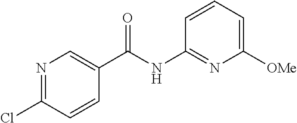 [SMILES: COc1cccc(NC(=O)c2ccc(Cl)nc2)n1]

HATU (838.7 mg, 2.20 mmol) was added to a mixture of 6-chloropyridine-3-carboxylic acid (315 mg, 2.0 mmol), 6-methoxypyridin-2-amine (248.3 mg, 2.0 mmol) and DIPEA (1.05 mL, 6.0 mmol) in DMF (4.0 mL) and the reaction stirred at rt for 18 h. The mixture was partitioned between EtOAc and water and the layers separated. The organic phase was washed with brine, dried over anhydrous MgSO₄ and filtered. The filtrate was concentrated in vacuo and the residue purified by Isco automatic purification system eluting with EtOAc/heptanes (40/60 to 0100/0) to afford 6-chloro-N-(6-methoxypyridin-2-yl)nicotinamide, 318 mg, 60.2% yield as a yellow solid. LCMS m/z=264.0 [M+H]⁺

### Preparation 85: tert-Butyl (4-methoxy-5-(pyridin-2-ylcarbamoyl)pyridin-2-yl)carbamate

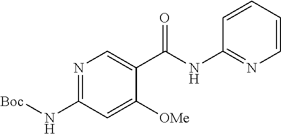 [SMILES: COc1cc(NC(=O)OC(C)(C)C)ncc1C(=O)Nc1ccccn1]

A vial containing a mixture of 6-chloro-4-methoxy-N-(pyridin-2-yl)nicotinamide (Preparation 79, 320.0 mg, 1.21 mmol), Pd(OAc)₂ (27.2 mg, 0.12 mmol), Xantphos (140.0 mg, 0.24 mmol), Cs₂CO₃ (788.5 mg, 2.42 mmol) and tert-butyl carbamate (708.8 mg, 6.05 mmol) was purged with N₂ and closed with a screw cap with septa. Dioxane (6.00 mL) was added, the vial sealed and the reaction heated at 100° C. for 18 h. The cooled reaction mixture was filtered through Celite® and the filtrate concentrated in vacuo. The crude product was purified using an Isco system eluting with EtOAc/Heptane (0/100 to 100/0) to provide tert-butyl (4-methoxy-5-(pyridin-2-ylcarbamoyl)pyridin-2-yl)carbamate, 100 mg, 24.0% yield, as a white solid. LCMS m/z=367.2 [M+H]⁺

### Preparations 86 to 90

The following compounds were prepared from the appropriate chloride and tert-butyl carbamate, following the procedure described in Preparation 85.

| | | Starting Material |
|---|---|---|
| Prep No | Structure and Name | Yield and data |
| 86 | 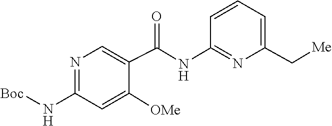 [SMILES: CCc1cccc(NC(=O)c2cnc(NC(=O)OC(C)(C)C)cc2OC)n1] | 6-chloro-N-(6-ethylpyridin-2-yl)-4- methoxynicotinamide (Preparation 80) white solid, 160 mg, 12.5% yield LCMS m/z = 373.2 [M + H]⁺ |
| | tert-butyl (5-((6-ethylpyridin-2- | |
| | yl)carbamoyl)-4-methoxypyridin- | |
| | 2-yl)carbamate | |
| 87 | 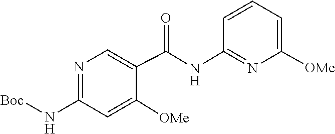 [SMILES: COc1cccc(NC(=O)c2cnc(NC(=O)OC(C)(C)C)cc2OC)n1] | 6-chloro-4-methoxy-N-(6- methoxypyridin-2-yl)nicotinamide (Preparation 81) white solid, 703.9 mg, 46% yield LCMS m/z = 397.3 [M + Na]⁺ |
| | tert-butyl (4-methoxy-5-((6- | |
| | methoxypyridin-2- | |
| | yl)carbamoyl)pyridin-2- | |
| | yl)carbamate | |
| 88^{A} | 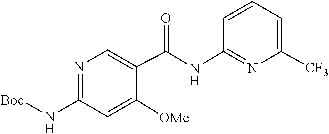 [SMILES: COc1cc(NC(=O)OC(C)(C)C)ncc1C(=O)Nc1cccc(C(F)(F)F)n1] | 6-chloro-4-methoxy-N-(6- (trifluoromethyl)pyridin-2- yl)nicotinamide (Preparation 82) light yellow solid. LCMS m/z = 357.1 [M − Bu]⁺ |
| | tert butyl (4-methoxy-5-((6- | |
| | (trifluoromethyl)pyridin-2- | |
| | yl)carbamoyl)pyridine-2- | |
| | yl)carbamate | |
| 89 | 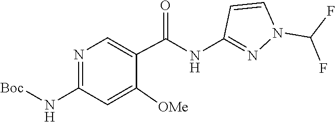 [SMILES: COc1cc(NC(=O)OC(C)(C)C)ncc1C(=O)Nc1ccn(C(F)F)n1] | SM: 6-chloro-N-(1-(difluoromethyl)- 1H-pyrazol-3-yl)-4- methoxynicotinamide (Preparation 83) |
| | tert butyl (5-((1-(difluoromethyl)- | |
| | 1H-pyrazol-3-yl)carbamoyl)-4- | |
| | methoxypyridin-2-yl)carbamate | |
| 90^{A} | 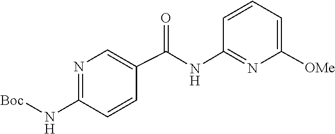 [SMILES: COc1cccc(NC(=O)c2ccc(NC(=O)OC(C)(C)C)nc2)n1] | 6-chloro-N-(6-methoxypyridin-2- yl)nicotinamide (Preparation 84) Light yellow solid, 61 mg, 46.8% yield LCMS m/z = 289.0 [M − Bu]⁺ |
| | tert-butyl (5-((6-methoxypyridin- | |
| | 2-yl)carbamoyl)pyridin-2- | |
| | yl)carbamate | |

^{A}0.2 equiv. Pd(OAc)₂ and 0.4 equiv. Xantphos used

### Preparation 91: tert-Butyl (5-((1-difluoromethyl)-1H-pyrazol-3-yl)carbamoyl)pyridin-2-yl)carbamate

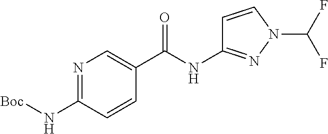 [SMILES: CC(C)(C)OC(=O)Nc1ccc(C(=O)Nc2ccn(C(F)F)n2)cn1]

HATU (838.7 mg, 2.20 mmol) was added to a mixture of 1-(difluoromethyl)pyrazol-3-amine hydrochloride (339 mg, 2.0 mmol), 6-(tert-butoxycarbonylamino)pyridine-3-carboxylic acid (476.5 mg, 2.0 mmol) and DIPEA (1.05 mL, 6.0 mmol) in DMF (5.0 mL) and the reaction mixture stirred at rt for 18 h. The mixture was partitioned between EtOAc and water and the layers separated. The organic phase was washed with brine, dried over anhydrous MgSO₄ and filtered. The filtrate was concentrated in vacuo and the residue purified by column chromatography on silica gel using an Isco automatic purification system eluting with EtOAc/heptanes (40/60 to 100/0) to afford tert-butyl (5-((1-difluoromethyl)-1H-pyrazol-3-yl)carbamoyl)pyridin-2-yl)carbamate, (375.0 mg, 53.0% yield) as a yellow solid. LCMS m/z=298.0 [M−Bu]⁺

### Preparation 92: 6-Amino-4-methoxy-N-(6-(trifluoromethyl)pyridin-2-yl)nicotinamide

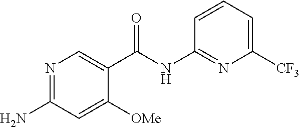 [SMILES: COc1cc(N)ncc1C(=O)Nc1cccc(C(F)(F)F)n1]

TFA (371 μL, 4.85 mmol) was added to a solution of tert butyl (4-methoxy-5-((6-(trifluoromethyl)pyridin-2-yl)carbamoyl)pyridine-2-yl)carbamate (Preparation 88, 200 mg, 0.485 mmol) in DCM (4 mL) and the reaction stirred at rt for 30 mins. The reaction was concentrated in vacuo and the crude material was purified by SCX ion exchange column eluting with MeOH/2N NH₃ in MeOH to afford 6-amino-4-methoxy-N-(6-(trifluoromethyl)pyridin-2-yl)nicotinamide (71.4 mg, 47.1% yield) as a white solid. LCMS m/z=313.0 [M+H]⁺

### Preparation 93: 6-Amino-4-methoxy-N-(pyridin-2-yl)nicotinamide trifluoroacetate

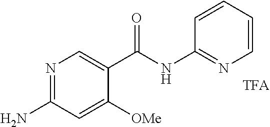 [SMILES: COc1cc(N)ncc1C(=O)Nc1ccccn1]

TFA (636 μL, 8.32 mmol) was added drop wise to a solution of tert-butyl (4-methoxy-5-(pyridin-2-ylcarbamoyl)pyridin-2-yl)carbamate (Preparation 85, 286 mg, 0.83 mmol) in DCM (2.0 mL) and the reaction stirred at rt for 30 mins. The mixture was evaporated under reduced pressure to afford 6-amino-4-methoxy-N-(pyridin-2-yl)nicotinamide trifluoroacetate, 629.0 mg, 92.9% yield. LCMS m/z=245.1 [M+H]⁺

### Preparations 94 to 98

The following compounds were prepared from the appropriate protected amine, according to the procedure described in Preparation 93.

| | | Starting Material |
|---|---|---|
| Prep No | Structure and Name | Yield and data |
| 94 | 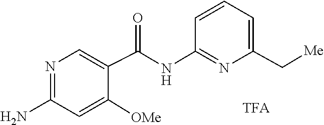 [SMILES: CCc1cccc(NC(=O)c2cnc(N)cc2OC)n1] | 400 mg, 97.8% yield SM: tert-butyl (5-((6-ethylpyridin-2- yl)carbamoyl)-4-methoxypyridin-2- yl)carbamate (Preparation 86) LCMS m/z = 273.1 [M + H]⁺ |
| | 6-amino-N-(6-ethylpyridin-2-yl)- | |
| | 4-methoxynicotinamide | |
| | trifluoroacetate | |
| 95 | 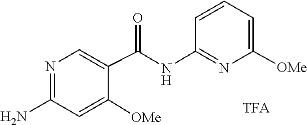 [SMILES: COc1cccc(NC(=O)c2cnc(N)cc2OC)n1] | 719 mg, 98.5% yield SM: tert-butyl (4-methoxy-5-((6- methoxypyridin-2- yl)carbamoyl)pyridin-2-yl)carbamate (Preparation 87) LCMS m/z = 297.0 [M + Na]⁺ |
| | 6-amino-4-methoxy-N-(6- | |
| | methoxypyridin-2- | |
| | yl)nicotinamide trifluoroacetate | |
| 96 | 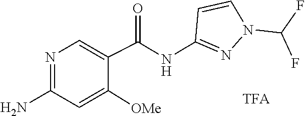 [SMILES: COc1cc(N)ncc1C(=O)Nc1ccn(C(F)F)n1] | SM: tert butyl (5-((1-(difluoromethyl)- 1H-pyrazol-3-yl)carbamoyl)-4- methoxypyridin-2-yl)carbamate (Preparation 89) |
| | 6-amino-N-(1-(difluoromethyl)- | |
| | 1H-pyrazol-3-yl)-4- | |
| | methoxynicotinamide | |
| | trifluoroacetate | |
| 97 | 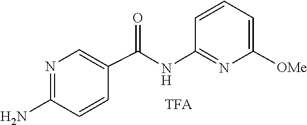 [SMILES: COc1cccc(NC(=O)c2ccc(N)nc2)n1] | white solid, 45.5 mg, 35.3% yield, SM: tert-butyl (5-((6-methoxypyridin- 2-yl)carbamoyl)pyridin-2-yl)carbamate (Preparation 90) LCMS m/z = 244.1 [M + H]⁺ |
| | 6-amino-N-(6-methoxypyridin-2- | |
| | yl)nicotinamide trifluoroacetate | |
| 98 | 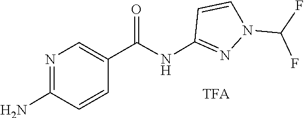 [SMILES: Nc1ccc(C(=O)Nc2ccn(C(F)F)n2)cn1] | SM: tert-butyl (5-((1-(difluoromethyl)- 1H-pyrazol-3-yl)carbamoyl)pyridin-2- yl)carbamate (Preparation 91) |
| | 6-amino-N-(1-(difluoromethyl)- | |
| | 1H-pyrazol-3-yl)nicotinamide | |
| | trifluoroacetate | |

### Preparation 99: tert-Butyl 3-(646-(difluoromethyl)pyridin-2-yl)carbamoyl)-7-ethoxyimidazo[1,2-a]pyridin-2-yl)azetidine-1-carboxylate

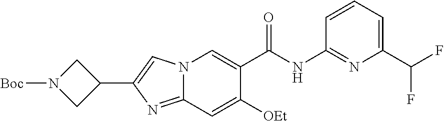 [SMILES: CCOc1cc2nc(C3CN(C(=O)OC(C)(C)C)C3)cn2cc1C(=O)Nc1cccc(C(F)F)n1]

To a solution of methyl 2-(1-(tert-butoxycarbonyl)azetidin-3-yl)-7-ethoxyimidazolo[1,2-a]pyridine-6-carboxylate (Preparation 52, 40 mg, 0.11 mmol) in MeOH (1 mL) and water (1 mL) was added NaOH (8.5 mg, 0.21 mmol) and the reaction stirred at 15° C. for 2 h. The mixture was concentrated in vacuo to remove MeOH and aqueous KHSO₄ was added to neutralise the solution. The mixture was evaporated under reduced pressure to give a white solid. To a solution of this compound (30 mg, 0.08 mmol), 6-(difluoromethyl)pyridin-2-amine (24 mg, 0.17 mmol) in pyridine (1 mL), was added T3P® (1 mL, 50% w/w in EtOAc) and the reaction stirred at rt for 14 h. The mixture was concentrated in vacuo, the residue diluted with aqueous NaHCO₃ (10 mL), extracted with EtOAc (30 mL×2), dried over Na₂SO₄, filtered and concentrated in vacuo. The residue was purified by prep-TLC eluting with DCM/MeOH (95/5) to afford tert-butyl 3-(6-((6-(difluoromethyl)pyridin-2-yl)carbamoyl)-7-ethoxyimidazo[1,2-a]pyridin-2-yl)azetidine-1-carboxylate, 93.3% as a yellow solid. LCMS m/z=488.2 [M+H]^{+ 1}H NMR (400 MHz, CDCl₃) δ: 1.47 (s, 9H), 1.70-1.74 (m, 3H), 3.85-3.95 (m, 1H), 4.12-4.19 (m, 2H), 4.31-4.37 (m, 4H), 6.37-6.65 (m, 1H), 6.98 (s, 1H), 7.40-7.44 (m, 2H), 7.88-7.93 (m, 1H), 8.47 (d, 1H), 9.02 (s, 1H), 10.60 (s, 1H)

### Preparation 100: 2-(Azetidin-3-yl)-N-(6-(difluoromethyl)pyridin-2-yl)-7-ethoxyimidazo[1,2-a]pyridine-6-carboxamide

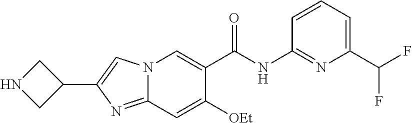 [SMILES: CCOc1cc2nc(C3CNC3)cn2cc1C(=O)Nc1cccc(C(F)F)n1]

To a solution of tert-butyl 3-(6-(((6-(difluoromethyl)pyridin-2-yl)carbamoyl)-7-ethoxyimidazo[1,2-a]pyridin-2-yl)azetidine-1-carboxylate (Preparation 99, 40 mg, 0.08 mmol) in DCM (1 mL) was added TFA (1 mL) and the reaction stirred at rt for 1 h. The mixture was concentrated in vacuo, the residue diluted with water (10 mL), neutralized using aq. NaHCO₃ and extracted with DCM (30 mL×3). The combined organic layers were dried over Na₂SO₄, filtered and evaporated under reduced pressure to afford 2-(azetidin-3-yl)-N-(6-(difluoromethyl)pyridin-2-yl)-7-ethoxyimidazo[1,2-a]pyridine-6-carboxamide 30 mg, 85% yield, as a white solid. ¹H NMR (500 MHz, CDCl₃) δ: 1.73 (t, 3H), 4.20-4.22 (m, 5H), 4.34-4.39 (m, 2H), 6.40-6.63 (m, 1H), 7.01 (s, 1H), 7.41-7.46 (m, 2H), 7.89-7.93 (m, 1H), 8.47 (d, 1H), 9.02 (s, 1H), 10.60 (s, 1H).

### Preparation 101: 3-Methoxy-3-methylbutyl 4-methylbenzenesulfonate

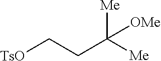 [SMILES: COC(C)(C)CCOS(=O)(=O)c1ccc(C)cc1]

TEA (513 mg, 5.07 mmol) and p-TsCl (483 mg, 2.54 mmol) were added to a solution of 3-methoxy-3-methyl-1-butanol (200 mg, 1.69 mmol) in DCM (10 mL) and the reaction stirred at 15° C. for 14 h. The reaction was washed with NaHCO₃ (15 mL×2), extracted with DCM (30 mL×2) and the combined organic layers dried over Na₂SO₄, filtered and concentrated in vacuo. The residue was purified by column chromatography on silica gel using a Combiflash® system, eluting with PE/EtOAc (75/25) to afford 3-methoxy-3-methylbutyl 4-methylbenzenesulfonate (320 mg, 66.0% yield) as yellow oil. ¹H NMR (400 MHz, CDCl₃) δ: 1.14 (s, 6H), 1.88 (t, 2H), 2.46 (s, 3H), 3.11 (s, 3H), 4.14 (t, 2H), 7.36 (d, 2H), 7.80 (d, 2H).

### Preparation 102: Methyl 6-amino-5-fluoro-4-isopropoxynicotinate

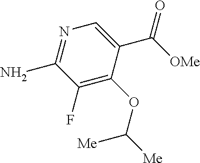 [SMILES: COC(=O)c1cnc(N)c(F)c1OC(C)C]

1-Chloromethyl-4-fluoro-1,4-diazoniabicyclo[2.2.2]octane bis(tetrafluoroborate) (2.53 g, 7.14 mmol) was added to a solution of methyl 6-amino-4-isopropoxynicotinate (500 mg, 2.38 mmol) in CHCl₃ (12 mL) and water (12 mL) and the reaction stirred for 18 h. The layers were separated, the organic phase dried, concentrated in vacuo, and purified by column chromatography to afford methyl 6-amino-5-fluoro-4-isopropoxynicotinate (118 mg, 21.7% yield). LCMS m/z=229.0 [M+H]^{+ 1}H NMR (500 MHz, CDCl₃) δ: 1.37 (dd, 6H) 3.85 (s, 3H) 4.71 (td, 1H) 5.26 (br s, 2H) 8.39 (s, 1H)

### Preparation 103: 5-Bromo-4-isopropoxypyrimidin-2-amine

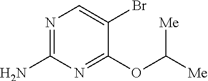 [SMILES: CC(C)Oc1nc(N)ncc1Br]

A mixture of 4-isopropoxypyrimidin-2-amine (5.90 g, 38.5 mmol) and NBS (6.86 g, 38.5 mmol) in CHCl₃ (257 mL) was stirred at rt for 18 h. The mixture was washed with aq. NaHCO₃ soln. and the organic layer evaporated under reduced pressure to afford 5-bromo-4-isopropoxypyrimidin-2-amine. LCMS m/z=232.0 [M+H]^{+ 1}H NMR (500 MHz, MeOH-d₄) δ: 1.35 (d, 6H), 5.39 (dq, 1H), 8.00 (s, 1H).

Preparation 104: Methyl 7-isopropoxyimidazo[1,2-a]pyridine-6-carboxylate

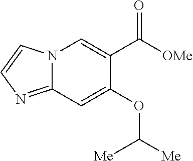 [SMILES: COC(=O)c1cn2ccnc2cc1OC(C)C]

A solution of methyl 6-amino-4-isopropoxynicotinamide (Preparation 2, 100 mg, 0.475 mmol) in MeCN (3 mL) was treated with 2-chloroacetaldehyde (157 μL, 1.24 mmol) and the reaction stirred at reflux for 1 h. The cooled mixture was acidified using 4 N HCl in dioxane (0.1 mL) then concentrated in vacuo. The crude product was dissolved in MeOH/H2O and purified by HPLC using a Hypersep™ SCX column, eluting with 2N NH₃/MeOH to provide methyl 7-isopropoxyimidazo[1,2-a]pyridine-6-carboxylate. LCMS m/z=235.0 [M+H]⁺

### Preparation 105: 2-Bromo-1-(bicyclo[1.1.1]pentan-1-yl)ethan-1-one

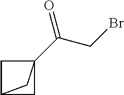

was obtained, from bicyclo[1.1.1]pentane-1-carboxylic acid, following the procedure described in Preparation 38.

### Preparation 106: 6-Bromo-8-ethoxy-2-(tetrahydro-2H-pyran-3-yl)imidazo[1,2-a]pyridine

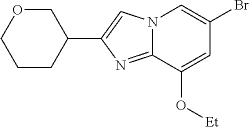 [SMILES: CCOc1cc(Br)cn2cc(C3CCCOC3)nc12]

A mixture of 5-bromo-3-ethoxypyridin-2-amine (1.0 g, 4.61 mmol), 2-bromo-1-(tetrahydro-2H-pyran-3-yl)ethan-1-one (954.5 mg, 4.61 mmol) and NaHCO₃ (1.16 g, 13.8 mmol) in MeCN (9.2 mL) was stirred at 80° C. for 18 h. The cooled reaction was filtered and the filtrate concentrated in vacuo. The crude product was purified by column chromatography on silica gel eluting with EtOAc/Heptanes (0/100 to 30/70) to obtain 6-bromo-8-ethoxy-2-(tetrahydro-2H-pyran-3-yl)imidazo[1,2-a]pyridine. LCMS m/z=324.9 [M+H]⁺

### Preparation 107: Methyl 2-(bicyclo[1.1.1]pentan-1-yl)-7-isopropoxyimidazo[1,2-a]pyridine-6-carboxylate

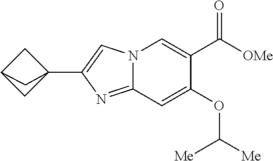 [SMILES: COC(=O)c1cn2cc(C34CC(C3)C4)nc2cc1OC(C)C]

To methyl 6-amino-4-isopropoxynicotinamide (Preparation 2, 111 mg, 0.529 mmol), 2-bromo-1-(bicyclo[1.1.1]pentan-1-yl)ethan-1-one (Preparation 105, 100 mg, 0.529 mmol), and NaHCO₃ (222 mg, 2.64 mmol) was added MeCN/toluene (V/V 1/1), (4 mL) at 100° C. The vial was sealed and heated at 100° C. for 18 h. The cooled reaction was filtered through a pad of Celite® and the filtrate concentrated in vacuo. The crude material was purified by column chromatography on silica gel using an Isco automated system, eluting with EtOAc/heptanes (0/100 to 100/0) to afford methyl 2-(bicyclo[1.1.1]pentan-1-yl)-7-isopropoxyimidazo[1,2-a]pyridine-6-carboxylate, (89.0 mg, 56.0% yield) as an off-white solid. LCMS m/z=301.2 [M+H]⁺.

### Preparations 108 to 114

The compounds in the following table were prepared from the appropriate amine and bromomethyl ketone, following the procedure described in Preparation 107.

| Prep. No | Structure and Name | Yield/Starting Materials/Data |
|---|---|---|
| 108 | 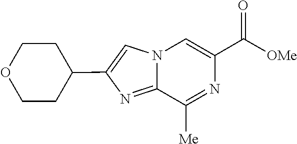 [SMILES: COC(=O)c1cn2cc(C3CCOCC3)nc2c(C)n1] | 300 mg, 52% from methyl 5-amino-6- methylpyrazine-2-carboxylate and 2- bromo-1-(tetrahydro-2H-pyran-4- yl)ethan-1-one LCMS m/z = 276.2 [M + H]⁺. |
| | Methyl 8-methyl-2-(tetrahydro- | |
| | 2H-pyran-4-yl)imidazo[1,2- | |
| | a]pyrazine-6-carboxylate | |
| 109 | 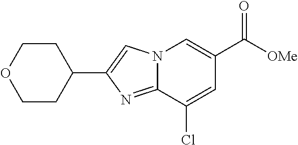 [SMILES: COC(=O)c1cc(Cl)c2nc(C3CCOCC3)cn2c1] | 280 mg, 50% from methyl 6-amino-5- chloronicotinate and 2-bromo-1- (tetrahydro-2H-pyran-4-yl)ethan-1-one LCMS m/z = 295.1 [M + H]⁺ |
| | methyl 8-chloro-2-(tetrahydro- | |
| | 2H-pyran-4-yl)imidazo[1,2- | |
| | a]pyridine-6-carboxylate | |
| 110 | 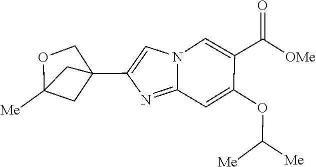 [SMILES: COC(=O)c1cn2cc(C34COC(C)(C3)C4)nc2cc1OC(C)C] | 1.43 g, 79.0% yield, from 6-amino-4- isopropoxynicotinamide (Preparation 2) and 2-bromo-1-(1-methyl-2- oxabicyclo[2.1.1]hexan-4-yl)ethan-1-one (Preparation 36) LCMS m/z = 331.1 [M + H]⁺ |
| | Methyl 7-isopropoxy-2-(1- | |
| | methyl-2- | |
| | oxabicyclo[2.1.1]hexan-4- | |
| | yl)imidazo[1,2-a]pyridine-6- | |
| | carboxylate | |
| 111 | 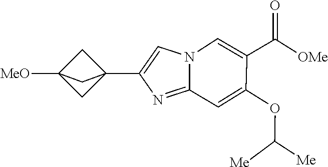 [SMILES: COC(=O)c1cn2cc(C34CC(OC)(C3)C4)nc2cc1OC(C)C] | 131.7 mg, 49.2% yield, from 6-amino-4- isopropoxynicotinamide (Preparation 2) and 2-bromo-1-(3- methoxybicyclo[1.1.1]pentan-1-yl)ethan- 1-one (Preparation 38) LCMS m/z = 331.2 [M + H]⁺ |
| | Methyl 7-isopropoxy-2-(3- | |
| | methoxybicyclo[1.1.1]pentan-1- | |
| | yl)imidazo[1,2-a]pyridine-6- | |
| | carboxylate | |
| 112 | 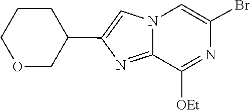 [SMILES: CCOc1nc(Br)cn2cc(C3CCCOC3)nc12] | 1.4 g, 93.5% as light yellow solid from 5-bromo-3-ethoxypyrazin-2-amine and 2-bromo-1-(tetrahydro-2H-pyran-3- yl)ethan-1-one LCMS m/z = 326.0 [M + H]⁺ |
| | 6-bromo-8-ethoxy-2-(tetrahydro- | |
| | 2H-pyran-3-yl)imidazo[1,2- | |
| | a]pyrazine | |
| 113 | 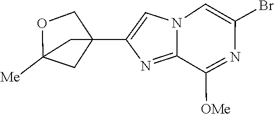 [SMILES: COc1nc(Br)cn2cc(C34COC(C)(C3)C4)nc12] | 230 mg, 56.3% yield as a brown oil from 5-bromo-3-methoxypyrazin-2-amine and 2-bromo-1-(1-methyl-2- oxabicyclo[2.1.1]hexan-4-yl)ethan-1-one (Preparation 36) LCMS m/z = 326.0 [M + H]⁺ |
| | 6-bromo-8-methoxy-2-(1-methyl- | |
| | 2-oxabicyclo[2.1.1]hexan-4- | |
| | yl)imidazo[1,2-a]pyrazine | |
| 114 | 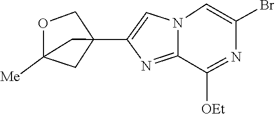 [SMILES: CCOc1nc(Br)cn2cc(C34COC(C)(C3)C4)nc12] | 283.4 mg, 91.4% yield, from 5-bromo-3- ethoxypyrazin-2-amine and 2-bromo-1- (1-methyl-2-oxabicyclo[2.1.1]hexan-4- yl)ethan-1-one (Preparation 36) LCMS m/z = 340.0 [M + H]⁺ |
| | 6-bromo-8-ethoxy-2-(1-methyl-2- | |
| | oxabicyclo[2.1.1]hexan-4- | |
| | yl)imidazo[1,2-a]pyrazine | |

### Preparation 115: Methyl 8-fluoro-7-isopropoxy-2-(1-methyl-2-oxabicyclo[2.1.1]hexan-4-yl)imidazo[1,2-a]pyridine-6-carboxylate

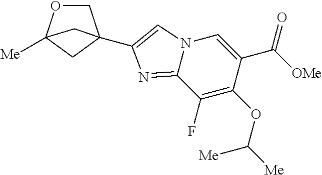 [SMILES: COC(=O)c1cn2cc(C34COC(C)(C3)C4)nc2c(F)c1OC(C)C]

A mixture of methyl 6-amino-5-fluoro-4-isopropoxynicotinate (Preparation 102, 140 mg, 0.613 mmol), 2-bromo-1-(1-methyl-2-oxabicyclo[2.1.1]hexan-4-yl)ethan-1-one (Preparation 36, 134 mg, 0.613 mmol) and NaHCO₃ (155 mg, 1.84 mmol) in EtOH (1.5 mL) was heated at 80° C. for 18 h. The cooled mixture was dry loaded onto silica gel and purified by column chromatography on silica gel to afford methyl 8-fluoro-7-isopropoxy-2-(1-methyl-2-oxabicyclo[2.1.1]hexan-4-yl)imidazo[1,2-a]pyridine-6-carboxylate (100 mg, 46.8% yield). LCMS m/z=349.0 [M+H]⁺

### Preparation 116: 6-Bromo-7-isopropoxy-2-(1-methyl-2-oxabicyclo[2.1.1]hexan-4-yl)imidazo[1,2-a]pyrimidine

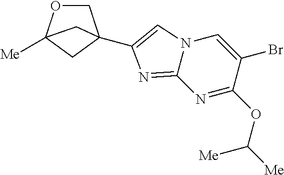 [SMILES: CC(C)Oc1nc2nc(C34COC(C)(C3)C4)cn2cc1Br]

To a solution of 2-bromo-1-(1-methyl-2-oxabicyclo[2.1.1]hexan-4-yl)ethan-1-one (Preparation 36, 25.6 g, 117 mmol) in toluene (140 mL) and MeCN (140 mL), 5-bromo-4-isopropoxypyrimidin-2-amine (27.1 g, 117 mmol) and NaHCO₃ (29.4 g, 350 mmol) were added and the reaction stirred at 95° C. (external) overnight. The cooled reaction mixture was filtered through Celite® and the filtrate concentrated in vacuo. The residue was purified by silica gel chromatography (heptane/EtOAc 100/0 to 20/80) to afford 6-bromo-7-isopropoxy-2-(1-methyl-2-oxabicyclo[2.1.1]hexan-4-yl)imidazo[1,2-a]pyrimidine as an orange solid (19.7 g, 48%). ¹H NMR (500 MHz, CDCl₃) δ: 1.44 (d, 6H) 1.53 (s, 3H) 1.93 (dd, 2H) 2.07 (s, 2H) 4.05 (s, 2H), 5.40-5.58 (m, 1H), 7.10 (s, 1H) 8.35 (s, 1H)

### Preparation 117: Methyl 2-(8-oxabicyclo[3.2.1]octan-3-yl)-7-isopropoxyimidazo[1,2-a]pyridine-6-carboxylate

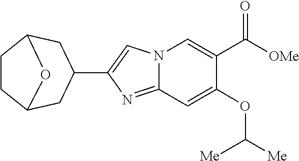 [SMILES: COC(=O)c1cn2cc(C3CC4CCC(C3)O4)nc2cc1OC(C)C]

A suspension of methyl 6-amino-4-isopropoxynicotinate (Preparation 2, 800 mg, 3.81 mmol), 1-(8-oxabicyclo[3.2.1]octan-3-yl)-2-chloroethan-1-one (Preparation 30, 1.08 g, 5.72 mmol) and NaHCO₃ (320 mg, 3.81 mmol) in MeOH (40 mL) was heated at 80° C. in a capped vial for 86 h. The cooled mixture was filtered and the filtrate concentrated in vacuo. The residue was purified by HPLC to afford methyl 2-(8-oxabicyclo[3.2.1]octan-3-yl)-7-isopropoxyimidazo[1,2-a]pyridine-6-carboxylate, 185 mg, 14.1% yield. LCMS m/z=345.4 [M+H]⁺

### Preparation 118: Phenyl 7-isopropoxy-2-(1-methyl-2-oxabicyclo[2.1.1]hexan-4-yl)imidazo[1,2-a]pyrimidine-6-carboxylate

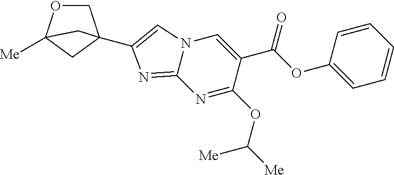 [SMILES: CC(C)Oc1nc2nc(C34COC(C)(C3)C4)cn2cc1C(=O)Oc1ccccc1]

TEA (22.0 mL, 0.16 mol) was added to a mixture of 6-bromo-7-isopropoxy-2-(1-methyl-2-oxabicyclo[2.1.1]hexan-4-yl)imidazo[1,2-a]pyrimidine (Preparation 116, 20.4 g, 57.9 mmol), Pd(OAc)₂ (1.30 g, 5.79 mmol), Xantphos (4.00 g, 6.91 mmol) and phenyl formate (18.0 g, 0.15 mol) in MeCN (120 mL) at rt and the reaction stirred at reflux overnight. The cooled mixture was filtered through Celite® and the filtrate concentrated in vacuo. The crude material was purified by silica gel chromatography (DCM/MeOH 100/0 to 95/5) to afford phenyl 7-isopropoxy-2-(1-methyl-2-oxabicyclo[2.1.1]hexan-4-yl)imidazo[1,2-a]pyrimidine-6-carboxylate as a brown oil (20.0 g, 88% yield). LCMS m/z=394.0 [M+H]^{+ 1}H NMR (500 MHz, CDCl₃) δ: 1.46 (d, 6H), 1.54 (s, 3H), 1.96 (dd, 2H), 2.07-2.17 (m, 2H), 4.08 (s, 2H), 5.63-5.65 (m, 1H), 7.18-7.51 (m, 6H), 9.04 (s, 1H)

### Preparation 119: Phenyl 8-ethoxy-2-(tetrahydro-2H-pyran-3-yl)imidazo[1,2-a]pyridine-6-carboxylate

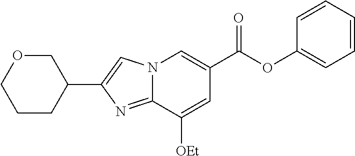 [SMILES: CCOc1cc(C(=O)Oc2ccccc2)cn2cc(C3CCCOC3)nc12]

Phenyl formate (269 mg, 2.20 mmol) followed by XantPhos-Pd-G3 (56.8 mg, 0.055 mmol) were added to a solution of 6-bromo-8-ethoxy-2-(tetrahydro-2H-pyran-3-yl)imidazo[1,2-a]pyridine (Preparation 106, 358 mg, 1.10 mmol) in MeCN (2.8 mL). TEA (223 mg, 2.20 mmol) was added and the reaction stirred under N₂ at 80° C. for 2 h. The cooled reaction was diluted with water, extracted with EtOAc, the phases separated and the organic layer washed with brine and dried over Na₂SO₄. The filtrate was concentrated in vacuo and the crude product purified by column chromatography on silica gel eluting with EtOAc/Heptanes (0/100 to 100/0) to afford phenyl 8-ethoxy-2-(tetrahydro-2H-pyran-3-yl)imidazo[1,2-a]pyridine-6-carboxylate. LCMS m/z=367.2 [M+H]⁺

### Preparation 120: Phenyl 8-ethoxy-2-(tetrahydro-2H-pyran-3-yl)imidazo[1,2-a]pyrazine-6-carboxylate

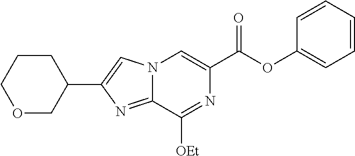 [SMILES: CCOc1nc(C(=O)Oc2ccccc2)cn2cc(C3CCCOC3)nc12]

TEA (1.49 mL, 10.7 mmol) was added to a mixture of 6-bromo-8-ethoxy-2-(tetrahydro-2H-pyran-3-yl)imidazo[1,2-a]pyrazine (Preparation 112, 1.40 g, 4.29 mmol), Pd(OAc)₂ (28.9 mg, 0.129 mmol), Xantphos (149 mg, 0.257 mmol) and phenyl formate (1.31 g, 10.7 mmol) in MeCN (12 mL) at rt and the sealed vial was heated at 80° C. for 18 h under N₂. The cooled reaction was filtered through a pad of Celite® and the filtrate concentrated in vacuo. The crude product was purified by column chromatography on silica gel eluting with (3:1 EtOAc:EtOH)/heptanes (0/100 to 50/50) to afford phenyl 8-ethoxy-2-(tetrahydro-2H-pyran-3-yl)imidazo[1,2-a]pyrazine-6-carboxylate (482 mg, 30.6% yield) as a white solid. LCMS m/z=368.3 [M+H]⁺

### Preparation 121: Phenyl 8-ethoxy-2-(1-methyl-2-oxabicyclo[2.1.1]hexan-4-yl)imidazo[1,2-a]pyrazine-6-carboxylate

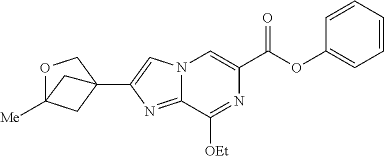 [SMILES: CCOc1nc(C(=O)Oc2ccccc2)cn2cc(C34COC(C)(C3)C4)nc12]

was obtained as a light yellow solid, 137 mg, 43.1% yield, from 6-bromo-8-ethoxy-2-(1-methyl-2-oxabicyclo[2.1.1]hexan-4-yl)imidazo[1,2-a]pyrazine (Preparation 114) and phenyl formate following the method described in Preparation 120. LCMS m/z=380.2 [M+H]⁺

### Preparation 122: Phenyl 8-methoxy-2-(1-methyl-2-oxabicyclo[2.1.1]hexan-4-yl)imidazo[1,2-a]pyrazine-6-carboxylate

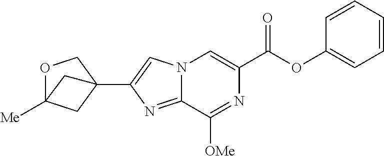 [SMILES: COc1nc(C(=O)Oc2ccccc2)cn2cc(C34COC(C)(C3)C4)nc12]

TEA (153 mg, 1.51 mmol) was added to a mixture of 6-bromo-8-methoxy-2-(1-methyl-2-oxabicyclo[2.1.1]hexan-4-yl)imidazo[1,2-a]pyrazine (Preparation 113, 196 mg, 0.605 mmol), Pd(OAc)₂ (9.50 mg, 0.042 mmol), Xantphos (28.0 mg, 0.048 mmol) and phenyl formate (184 mg, 1.51 mmol) in MeCN (2 mL) and the reaction heated at 80° C. for 5 h. The cooled mixture was partitioned between EtOAc and water and the layers separated. The organic extract was concentrated in vacuo and the residue purified by column chromatography on silica gel eluting with EtOAc/heptanes (50/50 to 70/30) to afford phenyl 8-methoxy-2-(1-methyl-2-oxabicyclo[2.1.1]hexan-4-yl)imidazo[1,2-a]pyrazine-6-carboxylate as an off-white solid, 151 mg. LCMS m/z=366.3 [M+H]⁺

### Preparation 123: 7-Isopropoxyimidazo[1,2-a]pyridine-6-carboxylic acid

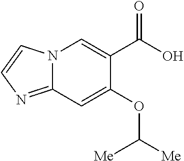 [SMILES: CC(C)Oc1cc2nccn2cc1C(=O)O]

LiOH (54 mg, 2.28 mmol) was added to a solution of methyl 7-isopropoxyimidazo[1,2-a]pyridine-6-carboxylate (Preparation 104, 107 mg, 0.457 mmol) in THF (3 mL), MeOH (0.5 mL) and water (1.3 mL) and the reaction stirred at rt for 18 h. The mixture was concentrated in vacuo, the residue acidified using 4N HCl in dioxane and then concentrated in vacuo. The crude product was purified by ion exchange chromatography using an SCX column, eluting with 2N NH₃/MeOH to afford 7-isopropoxyimidazo[1,2-a]pyridine-6-carboxylic acid. LCMS m/z=221.0 [M+H]⁺

### Preparation 124: 8-Ethoxy-2-(tetrahydro-2H-pyran-3-yl)imidazo[1,2-a]pyridine-6-carboxylic acid

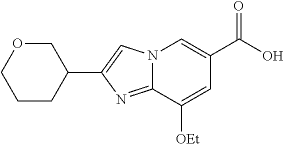 [SMILES: CCOc1cc(C(=O)O)cn2cc(C3CCCOC3)nc12]

was obtained from phenyl 8-ethoxy-2-(tetrahydro-2H-pyran-3-yl)imidazo[1,2-a]pyridine-6-carboxylate (Preparation 119), following a similar procedure to that described in Preparation 123. LCMS m/z=291.0 [M+H]⁺

### Preparation 125: 2-(Bicyclo[1.1.1]pentan-1-yl)-7-isopropoxyimidazo[1,2-a]pyridine-6-carboxylic acid

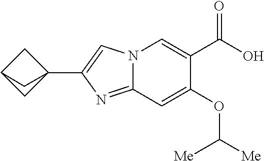 [SMILES: CC(C)Oc1cc2nc(C34CC(C3)C4)cn2cc1C(=O)O]

A mixture of methyl 2-(bicyclo[1.1.1]pentan-1-yl)-7-isopropoxyimidazo[1,2-a]pyridine-6-carboxylate (Preparation 107, 89 mg, 0.296 mmol) and NaOH (279 mg, 6.98 mmol) in MeOH (2 mL) and water (2 mL) was stirred at rt for 16 h in a sealed vessel. The mixture was neutralised using 1N aq. HCl and then concentrated in vacuo. The crude product was purified by prep-HPLC eluting with MeCN/0.1% aq. TFA (10/90 to 70/30) to provide 2-(bicyclo[1.1.1]pentan-1-yl)-7-isopropoxyimidazo[1,2-a]pyridine-6-carboxylic acid as a colourless oil. LCMS m/z=287.2 [M+H]⁺.

### Preparation 126: 7-Isopropoxy-2-(3-methoxybicyclo[1.1.1]pentan-1-yl)imidazo[1,2-a]pyridine-6-carboxylic acid

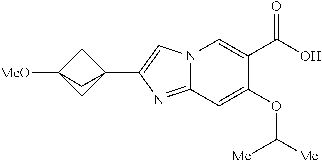 [SMILES: COC12CC(c3cn4cc(C(=O)O)c(OC(C)C)cc4n3)(C1)C2]

was obtained as a colorless oil, 112 mg, 89% yield from methyl 7-isopropoxy-2-(3-methoxybicyclo[1.1.1]pentan-1-yl)imidazo[1,2-a]pyridine-6-carboxylate (Preparation 111) following the procedure described in Preparation 125. LCMS m/z=317.1 [M+H]⁺

### Preparation 127: 8-Fluoro-7-isopropoxy-2-(1-methyl-2-oxabicyclo[2.1.1]hexan-4-yl)imidazo[1,2-a]pyridine-6-carboxylic acid

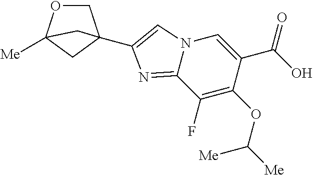 [SMILES: CC(C)Oc1c(C(=O)O)cn2cc(C34COC(C)(C3)C4)nc2c1F]

A mixture of methyl 8-fluoro-7-isopropoxy-2-(1-methyl-2-oxabicyclo[2.1.1]hexan-4-yl)imidazo[1,2-a]pyridine-6-carboxylate (Preparation 115, 110 mg, 0.280 mmol) in MeOH (932 H₂O (932 μL) and THF (932 μL) was treated with LiOH (20.1 mg, 0.839 mmol) and the reaction stirred for 2 h. The solution was acidified and evaporated under reduced pressure to afford 8-fluoro-7-isopropoxy-2-(1-methyl-2-oxabicyclo[2.1.1]hexan-4-yl)imidazo[1,2-a]pyridine-6-carboxylic acid. LCMS m/z=335.0 [M+H]⁺

### Preparation 128: 7-Isopropoxy-2-(1-methyl-2-oxabicyclo[2.1.1]hexan-4-yl)imidazo[1,2-a]pyrimidine-6-carboxylic acid

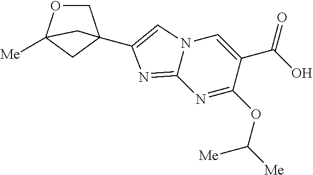 [SMILES: CC(C)Oc1nc2nc(C34COC(C)(C3)C4)cn2cc1C(=O)O]

LiOH.H₂O (2.55 g, 60.8 mmol) was added to a solution of phenyl 7-isopropoxy-2-(1-methyl-2-oxabicyclo[2.1.1]hexan-4-yl)imidazo[1,2-a]pyrimidine-6-carboxylate (Preparation 118, 20.0 g, 50.8 mmol) in THF (80 mL) and water (6 mL) and the reaction was stirred at rt overnight. 4 M HCl in dioxane (2.0 mL, 65.8 mmol) was added, the organic solvents were removed and the aqueous residue was stirred with heptanes: Et₂O 1:1 (100 mL) and then decanted. Et₂O (150 mL) and MeCN (50 mL) were added, the suspension was stirred for 2 h and the phases separated. The resulting precipitate was filtered off and washed with Et₂O to give 7-isopropoxy-2-(1-methyl-2-oxabicyclo[2.1.1]hexan-4-yl)imidazo[1,2-a]pyrimidine-6-carboxylic acid as an off-white solid (10.7 g, 57%). LCMS m/z=318.2 [M+H]⁺

### Preparation 129: 8-Methyl-2-(tetrahydro-2H-pyran-4-yl)imidazo[1,2-a]pyrazine-6-carboxylic acid

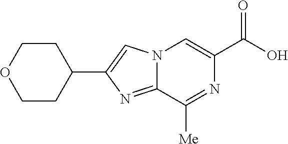 [SMILES: Cc1nc(C(=O)O)cn2cc(C3CCOCC3)nc12]

A mixture of methyl 8-methyl-2-(tetrahydro-2H-pyran-4-yl)imidazo[1,2-a]pyrazine-6-carboxylate (Preparation 108, 150 mg, 0.545 mmol) and LiOH.H₂O (45.7 mg, 1.09 mmol) in MeOH (0.4 mL), THF (3.3 mL) and water (0.8 mL) was stirred at 16 h at rt. The mixture was diluted with water and the pH adjusted to 2 using 4 M HCl. The aq. layer was extracted with EtOAc (3×), the combined organic extracts dried over MgSO₄, filtered, and evaporated under reduced pressure to afford 8-methyl-2-(tetrahydro-2H-pyran-4-yl)imidazo[1,2-a]pyrazine-6-carboxylic acid, 75 mg, 52% yield. LCMS m/z=262.2 [M+H]⁺

### Preparation 130: 8-Chloro-2-(tetrahydro-2H-pyran-4-yl)imidazo[1,2-a]pyridine-6-carboxylic acid

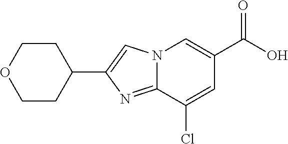 [SMILES: O=C(O)c1cc(Cl)c2nc(C3CCOCC3)cn2c1]

was obtained, 70 mg, 49% yield, from methyl 8-chloro-2-(tetrahydro-2H-pyran-4-yl)imidazo[1,2-a]pyridine-6-carboxylate (Preparation 109) following the procedure described in Preparation 129. LCMS m/z=281.1 [M+H]⁺

### Preparation 131: 8-Ethoxy-2-(tetrahydro-2H-pyran-3-yl)imidazo[1,2-a]pyrazine-6-carboxylic acid

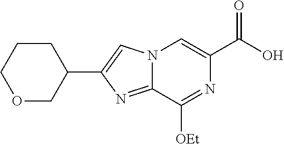 [SMILES: CCOc1nc(C(=O)O)cn2cc(C3CCCOC3)nc12]

was obtained as a yellow oil, in 96% yield, from phenyl 8-ethoxy-2-tetrahydropyran-3-yl-imidazo[1,2-a]pyrazine-6-carboxylate (Preparation 120) following a similar procedure to that described in Preparation 130. LCMS m/z=292.1 [M+H]⁺

### Preparation 132: 8-Methoxy-2-(1-methyl-2-oxabicyclo[2.1.1]hexan-4-yl)imidazo[1,2-a]pyrazine-6-carboxylic acid

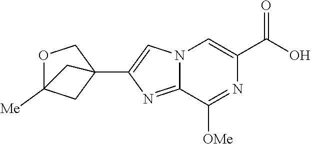 [SMILES: COc1nc(C(=O)O)cn2cc(C34COC(C)(C3)C4)nc12]

NaOH (1 M, 1 mL) was added to a solution of phenyl 8-methoxy-2-(1-methyl-2-oxabicyclo[2.1.1]hexan-4-yl)imidazo[1,2-a]pyrazine-6-carboxylate (Preparation 122, 148 mg, 0.405 mmol) in MeOH (2 mL) and THF (2 mL) and the reaction heated at 70° C. for 2 min, and then stirred at rt for 1.5 h. The mixture was acidified to pH 5 using 2N HCl, extracted with EtOAc (×3), and the combined organic extracts evaporated under reduced pressure to afford 8-methoxy-2-(1-methyl-2-oxabicyclo[2.1.1]hexan-4-yl)imidazo[1,2-a]pyrazine-6-carboxylic acid, 122 mg, as a white powder. LCMS m/z=290.1 [M+H]⁺

### Preparation 133: 8-Ethoxy-2-(1-methyl-2-oxabicyclo[2.1.1]hexan-4-yl)imidazo[1,2-a]pyrazine-6-carboxylic acid

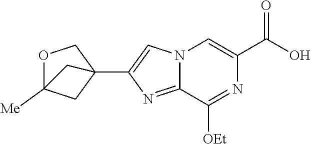 [SMILES: CCOc1nc(C(=O)O)cn2cc(C34COC(C)(C3)C4)nc12]

LiOH.H₂O (45.5 mg, 1.08 mmol) was added to a solution of phenyl 8-ethoxy-2-(1-methyl-2-oxabicyclo[2.1.1]hexan-4-yl)imidazo[1,2-a]pyrazine-6-carboxylate (Preparation 121, 137 mg, 0.361 mmol) in MeOH (2 mL) and H₂O (2 mL) and the reaction stirred at 22° C. for 16 h. The mixture was neutralised using 1M HCl and then concentrated in vacuo. The aqueous layer was extracted with EtOAc (10 mL×3), the combined organic layers were washed with brine (30 mL), dried over Na₂SO₄, and filtered. The filtrate was evaporated under reduced pressure to afford 8-ethoxy-2-(1-methyl-2-oxabicyclo[2.1.1]hexan-4-yl)imidazo[1,2-a]pyrazine-6-carboxylic acid, (108 mg, 98.5% yield) as a colorless oil. LCMS m/z=304.1 [M+H]⁺

### Preparation 134: tert-Butyl (5-bromo-(3-(difluoromethoxy)pyridin-2-yl)(tert-butoxycarbonyl)carbamate

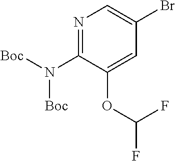 [SMILES: CC(C)(C)OC(=O)N(C(=O)OC(C)(C)C)c1ncc(Br)cc1OC(F)F]

To a solution of 5-bromo-3-(difluoromethoxy)pyridin-2-amine (3.00 g, 12.6 mmol) in DCM (31 mL) were added DMAP (1.53 g, 12.6 mmol), TEA (37.6 mmol, 5.2 mL) and Boc₂O (11.5 mL, 50.2 mmol) and the reaction stirred at rt for 18 h. The mixture was concentrated in vacuo and purified by silica gel column chromatography eluting with (3:1 EtOAC/EtOH)/heptanes (0/100 to 50/50) to afford tert-butyl (5-bromo-(3-(difluoromethoxy)pyridin-2-yl)(tert-butoxycarbonyl)carbamate (4.2 g, 76% yield). LCMS m/z=284.9 [M−Boc−tBu+H]⁺.

### Preparation 135: Phenyl 6-(bis(tert-butoxycarbonyl)amino)-5-(difluoromethoxy)nicotinate

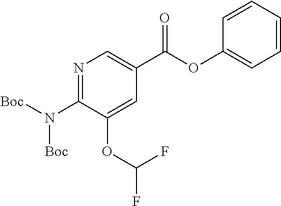 [SMILES: CC(C)(C)OC(=O)N(C(=O)OC(C)(C)C)c1ncc(C(=O)Oc2ccccc2)cc1OC(F)F]

TEA (790 μL, 5.70 mmol) was added to a mixture of tert-butyl (5-bromo-(3-(difluoromethoxy)pyridin-2-yl)(tert-butoxycarbonyl)carbamate (Preparation 134, 1.0 g, 2.28 mmol), Pd(OAc)₂ (15 mg, 0.068 mmol), Xantphos (79.1 mg, 0.137 mmol) and phenyl formate (621 μL, 5.70 mmol) in MeCN (6.5 mL) and the reaction was stirred at 80° C. for 16 h. The cooled mixture was evaporated under reduced pressure. The crude product was purified by silica gel column chromatography eluting with (3:1 EtOAC/EtOH)/heptanes (0/100 to 50/50) to afford phenyl 6-(bis(tert-butoxycarbonyl)amino)-5-(difluoromethoxy)nicotinate (1.00 g, 91% yield). LCMS m/z=325.2 [M−Boc−tBu+H]⁺

### Preparation 136: 6-((tert-Butoxycarbonyl)amino)-5-(difluoromethoxy)nicotinic acid

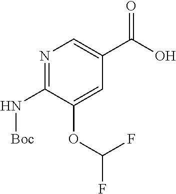 [SMILES: CC(C)(C)OC(=O)Nc1ncc(C(=O)O)cc1OC(F)F]

was obtained in 79% yield from phenyl 6-(bis(tert-butoxycarbonyl)amino)-5-(difluoromethoxy)nicotinate (Preparation 135) following the procedure described in Preparation 129. LCMS m/z=249.1 [M−tBu+H]⁺

### Preparation 137: tert-Butyl (3-(difluoromethoxy)-5-((6-(difluoromethyl)pyridin-2-yl)carbamoyl)pyridin-2-yl)carbamate

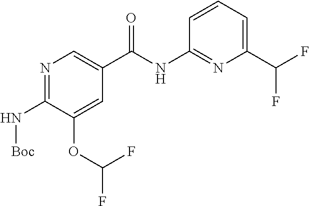 [SMILES: CC(C)(C)OC(=O)Nc1ncc(C(=O)Nc2cccc(C(F)F)n2)cc1OC(F)F]

To a mixture of 6-(difluoromethyl)pyridin-2-amine hydrochloride (373 mg, 2.07 mmol) and 6-((tert-butoxycarbonyl)amino)-5-(difluoromethoxy)nicotinic acid (Preparation 136, 420 mg, 1.38 mmol) in pyridine (4.6 mL) was added T3P® (50% EtOAc solution, 4.1 mL, 6.9 mmol) and the reaction stirred at rt for 2 h. The mixture was diluted with water, extracted with EtOAc (3×), the combined organic extracts washed with brine, dried over MgSO₄, filtered, and evaporated under reduced pressure to afford tert-butyl (3-(difluoromethoxy)-5-((6-(difluoromethyl)pyridin-2-yl)carbamoyl)pyridin-2-yl)carbamate. LCMS m/z=375.1 [M−Boc+H]⁺

### Preparation 138: 6-Amino-5-(difluoromethoxy)-N-(6-(difluoromethyl)pyridin-2-yl)nicotinamide

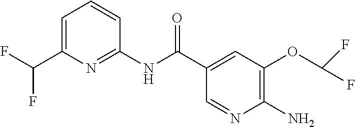 [SMILES: Nc1ncc(C(=O)Nc2cccc(C(F)F)n2)cc1OC(F)F]

TFA (10.5 mmol, 0.8 mL) was added to a solution of tert-butyl (3-(difluoromethoxy)-5-((6-(difluoromethyl)pyridin-2-yl)carbamoyl)pyridin-2-yl)carbamate (Preparation 137, 451 mg, 1.05 mmol) in DCM (4.2 mL) and the reaction stirred for 16 h. The reaction mixture was concentrated in vacuo, partitioned between EtOAc and NaHCO₃ and the layers separated. The aqueous layer was extracted with EtOAc (3×), the combined organic extracts washed with brine, dried over MgSO₄, filtered, and evaporated under reduced pressure to afford 6-amino-5-(difluoromethoxy)-N-(6-(difluoromethyl)pyridin-2-yl)nicotinamide (300 mg, 86% yield). LCMS m/z=331.1 [M+H]⁺

### Preparation 139: 3-Amino-5-fluoro-1-methylpyridin-2(1H)-one

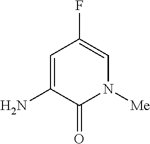

Zinc (2.97 g, 45.5 mmol) was added to a mixture of 5-fluoro-1-methyl-3-nitro-pyridin-2-one (559 mg, 3.25 mmol) and NH₄Cl (2.43 g, 45.47 mmol) in MeOH (24 mL) and THF (8 mL) and the reaction stirred at rt for 30 min. The reaction was diluted with EtOAc (20 mL), filtered through Celite®, and the filtrate evaporated under reduced pressure. Water (10 mL) was added, the mixture extracted with DCM (3×20 mL) and the combined extracts were dried over MgSO₄ and filtered. The filtrate was evaporated under reduced pressure to afford 3-amino-5-fluoro-1-methylpyridin-2(1H)-one as a brown solid (436.0 mg, 94.4% yield). LCMS m/z=143.0 [M+H]⁺

### Preparation 140: 2-Chloro-1-(4-oxaspiro[2.5]octan-1-yl)ethan-1-one

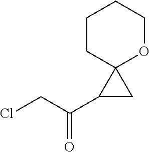 [SMILES: O=C(CCl)C1CC12CCCCO2]

was prepared from 4-oxaspiro[2.5]octane-1-carboxylic acid following the procedure described in Preparation 6.

### Preparation 141: 2-Chloro-1-(4-oxaspiro[2.5]octan-1-yl)ethan-1-one

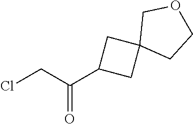 [SMILES: O=C(CCl)C1CC2(CCOC2)C1]

was prepared from 6-oxaspiro[3.4]octane-2-carboxylic acid, following the procedure described in Preparation 6.

### Preparation 142: 2-Bromo-6-(1,2-difluoroethyl)pyridine

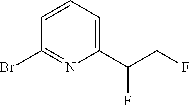 [SMILES: FCC(F)c1cccc(Br)n1]

To a solution of 1-(6-bromopyridin-2-yl)ethane-1,2-diol (1.60 g, 7.39 mmol) in DCM (30 mL) was added DAST (2.84 g, 12.8 mmol) at 0° C. and the reaction stirred at 15-20° C. for 16 h. The reaction was quenched with saturated aq. NaHCO₃ (30 mL) and extracted with DCM (30 mL×3). The combined organic layers were washed with brine (50 mL), dried over Na₂SO₄ and filtered. The filtrate was concentrated in vacuo and the residue purified by silica gel column chromatography using a Combiflash® system, eluting with (PE/EtOAc=10/1 to 3/1) to afford 2-bromo-6-(1,2-difluoroethyl)pyridine (500 mg, 44% yield) as yellow oil. ¹H NMR (500 MHz, CDCl₃) δ: 4.70-5.00 (m, 2H), 5.70-5.80 (m, 1H), 7.40-7.50 (m, 1H), 7.50-7.60 (m, 1H), 7.60-7.70 (m, 1H).

### Preparation 143: tert-Butyl (6-(1,2-difluoroethyl)pyridin-2-yl)carbamate

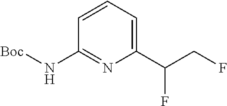 [SMILES: CC(C)(C)OC(=O)Nc1cccc(C(F)CF)n1]

To a solution of 2-bromo-6-(1,2-difluoroethyl)pyridine (Preparation 142, 50 mg, 0.23 mmol) and tert-butyl carbamate (40 mg, 0.34 mmol) in toluene (3 mL) were added Pd₂(dba)₃ (21 mg, 0.023 mmol), Xantphos (26 mg, 0.045 mmol) and Cs₂CO₃ (147 mg, 0.450 mmol), the mixture degassed with N₂ and the reaction stirred at 100° C. for 16 h. The cooled reaction mixture was concentrated in vacuo and the residue purified by Combiflash® (PE/EtOAc=20/1 to 10/1) to afford the title compound, 100 mg, as a yellow solid. LCMS m/z=202.8 [M−Boc+H]⁺

### Preparation 144: tert-Butyl (6-(oxazol-5-yl)pyridin-2-yl)carbamate

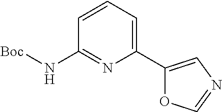 [SMILES: CC(C)(C)OC(=O)Nc1cccc(-c2cnco2)n1]

was obtained as a yellow solid, 560 mg, 86.9% yield, from 5-(6-bromopyridin-2-yl)oxazole, following a similar procedure to that described in Preparation 143. LCMS m/z=205.9 [M−tBu+H]⁺

### Preparation 145: tert-Butyl (6-vinylpyrazolo[1,5-a]pyrimidin-3-yl)carbamate

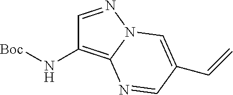 [SMILES: C=Cc1cnc2c(NC(=O)OC(C)(C)C)cnn2c1]

To a solution of tert-butyl (6-bromopyrazolo[1,5-a]pyrimidin-3-yl)carbamate (800 mg, 2.55 mmol) and 4,4,5,5-tetramethyl-2-vinyl-1,3,2-dioxaborolane (590 mg, 3.83 mmol) in dioxane (5 mL) and water (2 mL) was added Pd(dppf)Cl₂ (187 mg, 0.255 mmol) and K₂CO₃ (1.06 g, 7.66 mmol), the mixture degassed with N₂ and the reaction stirred at 90° C. for 16 h. The reaction was diluted with water (20 mL) and extracted with EtOAc (30 mL×3). The combined organic layers were washed with brine (50 mL), dried over Na₂SO₄ and filtered. The filtrate was concentrated in vacuo and the residue purified by Combiflash® (PE/EtOAc=10/1 to 3/1) to afford tert-butyl (6-vinylpyrazolo[1,5-a]pyrimidin-3-yl)carbamate (600 mg, 90.4% yield) as a yellow solid. ¹H NMR (500 MHz, CDCl₃) δ: 1.55 (s, 9H), 5.44 (d, 1H), 5.86 (d, 1H), 6.60-6.70 (m, 1H), 6.82 (br s, 1H), 8.46 (s, 1H), 8.50-8.60 (m, 1H).

### Preparation 146: tert-Butyl (6-formylpyrazolo[1,5-a]pyrimidin-3-yl)carbamate

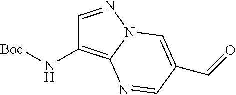 [SMILES: CC(C)(C)OC(=O)Nc1cnn2cc(C=O)cnc12]

To a solution of tert-butyl (6-vinylpyrazolo[1,5-a]pyrimidin-3-yl)carbamate (Preparation 145, 200 mg, 0.768 mmol) in dioxane (3 mL) and water (1 mL) was added K₂OsO₄ (28 mg, 0.077 mmol) and NaIO₄ (575 mg, 2.69 mmol) and the reaction stirred at 15° C. for 2 h. The reaction was quenched with water (50 mL) and extracted with EtOAc (50 mL×3). The combined organic layers were washed with brine (50 mL), dried over Na₂SO₄ and filtered. The filtrate was concentrated in vacuo and the residue purified by Combiflash® (PE/EtOAc=10/1 to 1/1) to afford tert-butyl (6-formylpyrazolo[1,5-a]pyrimidin-3-yl)carbamate (200 mg, 99.2% yield) as a yellow solid. ¹H NMR (400 MHz, CDCl₃) δ: 1.56 (s, 9H), 6.92 (s, 1H), 8.67 (s, 1H), 8.83 (s, 1H), 8.97 (s, 1H), 9.98 (s, 1H).

### Preparation 147: tert-Butyl (6-(difluoromethyl)pyrazolo[1,5-a]pyrimidin-3-yl)carbamate

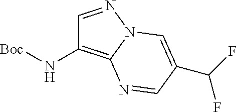 [SMILES: CC(C)(C)OC(=O)Nc1cnn2cc(C(F)F)cnc12]

was obtained as a yellow solid, 100 mg, 46.1% yield, from tert-butyl (6-formylpyrazolo[1,5-a]pyrimidin-3-yl)carbamate (Preparation 146), following the procedure described in Preparation 142. ¹H NMR (500 MHz, CDCl₃) δ: 1.56 (s, 9H), 6.70-6.80 (m, 2H), 8.44 (s, 1H), 8.70-8.80 (m, 2H)

### Preparation 148: 6-(Difluoromethyl)pyrazolo[1,5-a]pyrimidin-3-amine 2,2,2 trifluoroacetate

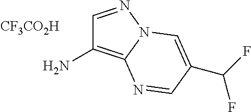 [SMILES: "CF3CO2H" ; Nc1cnn2cc(C(F)F)cnc12]

To a solution of tert-butyl (6-(difluoromethyl)pyrazolo[1,5-a]pyrimidin-3-yl)carbamate (Preparation 147, 50 mg, 0.176 mmol) in DCM (1 mL) was added TFA (0.5 mL) and the reaction stirred at 15° C. for 1 h. The mixture was evaporated under reduced pressure to afford 6-(difluoromethyl)pyrazolo[1,5-a]pyrimidin-3-amine 2,2,2 trifluoroacetate (30 mg, 92.6% yield) as a yellow solid. LCMS m/z=184.9 [M+H]⁺

### Preparation 149: 6-(1,2-Difluoroethyl)pyridin-2-amine

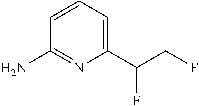 [SMILES: Nc1cccc(C(F)CF)n1]

To a solution of tert-butyl (6-(1,2-difluoroethyl)pyridin-2-yl)carbamate (Preparation 143, 100 mg) in DCM (2 mL) was added TFA (10.0 mg, 0.085 mmol) and the reaction stirred at 20° C. for 1 h. The reaction mixture was concentrated in vacuo, the residue diluted with saturated aq. NaHCO₃ (20 mL) and extracted with EtOAc (20 mL×3). The combined organic layers were washed with brine (30 mL), dried over Na₂SO₄ and filtered. The filtrate was evaporated under reduced pressure to afford 6-(1,2-difluoroethyl)pyridin-2-amine, 20.0 mg as a yellow oil. LCMS m/z=159.1 (M+H)⁺

### Preparation 150: 6-(Oxazol-5-yl)pyridin-2-amine 2,2,2-trifluoroacetate

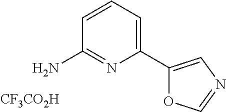 [SMILES: "CF3CO2H" ; Nc1cccc(-c2cnco2)n1]

A solution of tert-butyl (6-(oxazol-5-yl)pyridin-2-yl)carbamate (Preparation 144, 50 mg, 0.191 mmol) in TFA (1 mL) and DCM (2 mL) was stirred at 25° C. for 4 h. The mixture was concentrated in vacuo and the residue purified by Combiflash® (PE/EtOAc=3/1) to afford 6-(oxazol-5-yl)pyridin-2-amine 2,2,2-trifluoroacetate (30 mg, 87.5% yield) as a white solid. LCMS m/z=162.1 [M+H]⁺

### Preparation 151: tert-Butyl (6-fluoropyrazolo[1,5-a]pyrimidin-3-yl)carbamate

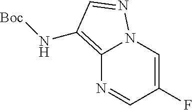 [SMILES: CC(C)(C)OC(=O)Nc1cnn2cc(F)cnc12]

To a solution of 6-fluoropyrazolo[1,5-a]pyrimidine-3-carboxylic acid (100 mg, 0.442 mmol) in t-BuOH (5 mL) was added DPPA (145.8 mg, 0.530 mmol) and TEA (89.4 mg, 0.883 mmol) and the reaction stirred at 100° C. for 16 h. The reaction mixture was diluted with water (30 mL) and extracted with EtOAc (30 mL×3). The combined organic layers were washed with brine (50 mL), dried over Na₂SO₄ and filtered. The filtrate was concentrated in vacuo and the residue purified by silica gel column chromatography using a Combiflash® system and eluting with (PE/EtOAc=10/1 to 1/1) to afford tert-butyl (6-fluoropyrazolo[1,5-a]pyrimidin-3-yl)carbamate (30 mg, 26.9% yield) as a yellow solid. LCMS m/z=252.9 [M+H]⁺

### Preparation 152: tert-Butyl (6-methoxypyrazolo[1,5-a]pyrimidin-3-yl)carbamate

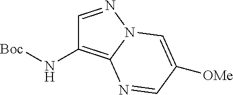 [SMILES: COc1cnc2c(NC(=O)OC(C)(C)C)cnn2c1]

was obtained as a brown solid, 150 mg, 13.3% yield, from 6-methoxypyrazolo[1,5-a]pyrimidine-3-carboxylic acid, following the procedure described in Preparation 151. LCMS m/z=208.8 [M−Boc+H]⁺

### Preparation 153: tert-Butyl pyrrolo[2,1-f][1,2,4]triazin-7-ylcarbamate

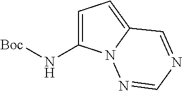 [SMILES: CC(C)(C)OC(=O)Nc1ccc2cncnn12]

was prepared as a white solid, from pyrrolo[2,1-f][1,2,4]triazine-7-carboxylic acid, 120 mg, 37.9% yield, following a similar procedure to that described in Preparation 151. LCMS m/z=234.9 [M+H]⁺

### Preparation 154: 6-Fluoropyrazolo[1,5-a]pyrimidin-3-amine hydrochloride

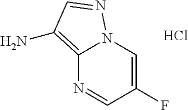 [SMILES: Nc1cnn2cc(F)cnc12 ; Cl]

To a solution of tert-butyl (6-fluoropyrazolo[1,5-a]pyrimidin-3-yl)carbamate (Preparation 151, 30 mg, 0.119 mmol) in EtOAc (2 mL) was added HCl/EtOAc (4 M, 2 mL) and the solution stirred at 15° C. for 1 h. The mixture was evaporated under reduced pressure to afford 6-fluoropyrazolo[1,5-a]pyrimidin-3-amine hydrochloride as a yellow solid (22.0 mg). LCMS m/z=152.9 [M+H]⁺

### Preparation 155: Pyrrolo[2,1-f][1,2,4]triazin-7-amine hydrochloride

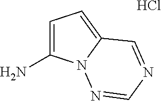

was obtained as a yellow solid, from tert-butyl pyrrolo[2,1-f][1,2,4]triazin-7-ylcarbamate (Preparation 153) following the procedure described in Preparation 154. LCMS m/z=135.1 [M+H]⁺

### Preparation 156: 6-Methoxypyrazolo[1,5-a]pyrimidin-3-amine

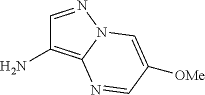 [SMILES: COc1cnc2c(N)cnn2c1]

A solution of tert-butyl (6-methoxypyrazolo[1,5-a]pyrimidin-3-yl)carbamate (Preparation 152, 130 mg, 0.517 mmol) in EtOAc/HCl (5 mL) was stirred at 20° C. for 16 h. The mixture was concentrated in vacuo, the residue neutralised using aq. NaHCO₃ and the mixture extracted with EtOAc (20 mL×3). The combined organic layers were washed with brine (20 mL), dried over Na₂SO₄, filtered and concentrated in vacuo. The residue was purified by column chromatography on silica gel (from PE/EtOAc=5/1 to 1/1) to afford 6-methoxypyrazolo[1,5-a]pyrimidin-3-amine (70 mg, 76.1% yield) as a brown solid. LCMS m/z=165.2 [M+H]⁺

### Preparation 157: 6-Methoxy-3-nitroimidazo[1,2-b]pyridazine

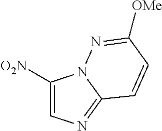 [SMILES: COc1ccc2ncc([N+](=O)[O-])n2n1]

A solution of 6-chloro-3-nitro-imidazo[1,2-b]pyridazine (1.0 g, 5.04 mmol) in NaOMe (4.37 M in MeOH, 4.61 mL) was stirred at rt. The solution was diluted with sat NH₄C1, extracted with EtOAc, and the combined organic extracts evaporated under reduced pressure to afford 6-methoxy-3-nitroimidazo[1,2-b]pyridazine. LCMS m/z=194.9 [M+H]⁺

### Preparation 158: 5-Methoxy-3-nitropyrazolo[1,5-a]pyrimidine

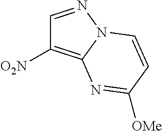 [SMILES: COc1ccn2ncc([N+](=O)[O-])c2n1]

obtained from 5-chloro-3-nitro-pyrazolo[1,5-a]pyrimidine following the procedure described in Preparation 157. LCMS m/z=194.9 [M+H]⁺

Preparation 159: 6-Methoxyimidazo[1,2-b]pyridazin-3-amine

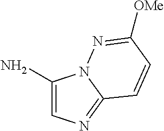 [SMILES: COc1ccc2ncc(N)n2n1]

Fe (2.88 g, 51.50 mmol) and NH₄Cl (2.75 g, 51.50 mmol) were added to a solution of 6-methoxy-3-nitroimidazo[1,2-b]pyridazine (Preparation 157, 999.8 mg, 5.15 mmol) in EtOH (58.52 mL) and H₂O (5.85 mL) and the reaction stirred at 80° C. for 4 h. The cooled mixture was filtered through Celite®, and the filtrate extracted with EtOAc (50 mL×3). The combined organic layers were dried over Na₂SO₄ and evaporated under reduced pressure to afford the 6-methoxyimidazo[1,2-b]pyridazin-3-amine. LCMS m/z=165.0 [M+H]⁺

### Preparation 160: 5-Methoxypyrazolo[1,5-a]pyrimidin-3-amine

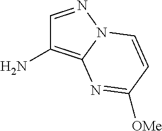 [SMILES: COc1ccn2ncc(N)c2n1]

was obtained from 5-methoxy-3-nitropyrazolo[1,5-a]pyrimidine (Preparation 158) following the procedure described in Preparation 159. LCMS m/z=165.0 [M+H]⁺

### Preparation 161: 5-(Difluoromethyl)pyrazolo[1,5-a]pyrimidin-3-amine

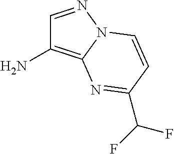 [SMILES: Nc1cnn2ccc(C(F)F)nc12]

was obtained from 5-(difluoromethyl)-3-nitro-pyrazolo[1,5-a]pyrimidine, following the procedure described in Preparation 159.

### Preparation 162: Benzyl (4-methoxypyrazolo[1,5-a]pyridin-3-yl)carbamate

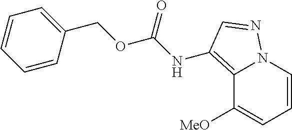 [SMILES: COc1cccn2ncc(NC(=O)OCc3ccccc3)c12]

To a solution of 4-methoxypyrazolo[1,5-a]pyridine-3-carboxylic acid (500 mg, 2.60 mmol) in THF (4 mL) and DIPEA (739.2 mg, 5.72 mmol) under an N₂ atmosphere, was added DPPA (787.1 mg, 2.86 mmol) and the reaction stirred at 20° C. for 16 h. The reaction was evaporated under reduced pressure to give 3-isocyanato-4-methoxy-pyrazolo[1,5-a]pyridine. A solution of 3-isocyanato-4-methoxy-pyrazolo[1,5-a]pyridine (490 mg, 2.59 mmol) in benzyl alcohol (232.3 mg, 5.18 mmol) was stirred at reflux for 16 h. The cooled reaction mixture was diluted with water (50 mL) and extracted with EtOAc (100 mL×3). The combined organic extracts were washed with saturated aq. NaHCO₃ (10 mL), dried over anhydrous Na₂SO₄, filtered and concentrated in vacuo. The residue was purified by column chromatography on silica gel (PE/EtOAc=1/1) to afford benzyl (4-methoxypyrazolo[1,5-a]pyridin-3-yl)carbamate (520 mg, 60.8% yield) as a white solid. LCMS m/z=297.2 [M+H]⁺

### Preparation 163: 4-Methoxypyrazolo[1,5-a]pyridin-3-amine

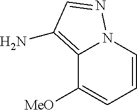 [SMILES: COc1cccn2ncc(N)c12]

To solution of benzyl (4-methoxypyrazolo[1,5-a]pyridin-3-yl)carbamate (Preparation 162, 520 mg, 1.75 mmol) in MeOH (3.50 mL) and EtOAc (3.50 mL) was added Pd/C (186.1 mg, 1.75 mmol) at 20° C. under Ar. The mixture was stirred at 20° C. under 15 psi of H₂ for 4 h. The reaction mixture was filtered through Celite®, the filtrate was evaporated under reduced pressure to afford 4-methoxypyrazolo[1,5-a]pyridin-3-amine (180 mg, crude) as a white solid. LCMS m/z=164.2 [M+H]⁺

### Preparation 164: 1-(Fluoromethyl)-N-methoxy-N-methyl-2-oxabicyclo[2.1.1]hexane-4-carboxamide

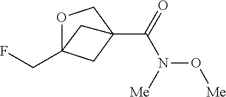 [SMILES: CON(C)C(=O)C12COC(CF)(C1)C2]

CDI (1.21 g, 7.49 mmol) was added to 4-(fluoromethyl)-3-oxabicyclo[2.1.1]hexane-1-carboxylic acid (1.00 g, 6.24 mmol) in DCM (10.4 mL), the solution stirred at rt for 2 h, then N,O-dimethylhydroxylamine hydrochloride (609.1 mg, 6.24 mmol) added and the reaction stirred at rt overnight. The mixture was poured into ice water, extracted with DCM, the combined organic extracts washed with brine, dried over MgSO₄, filtered and concentrated in vacuo. The residue was purified by silica gel column chromatography (0-100% 3:1 EtOAc:EtOH in heptanes) to obtain 1-(fluoromethyl)-N-methoxy-N-methyl-2-oxabicyclo[2.1.1]hexane-4-carboxamide (900 mg, 71.0% yield) as a white solid. LCMS m/z=204.1 [M+H]⁺

### Preparation 165: 1-(1-(Fluoromethyl)-2-oxabicyclo[2.1.1]hexan-4-yl)ethan-1-one

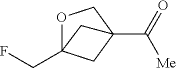 [SMILES: CC(=O)C12COC(CF)(C1)C2]

MeLi (1.6 M, 3.46 mL) was added to a solution of 4-(fluoromethyl)-N-methoxy-N-methyl-3-oxabicyclo[2.1.1]hexane-1-carboxamide (Preparation 164, 900 mg, 4.43 mmol) in THF (8.86 mL) at −78° C. under N₂, the reaction stirred for 30 min at 0° C., then allowed to warm to rt over 2 h. The reaction was quenched with NH₄Cl, extracted with EtOAc (3×), the combined organic extracts washed with brine, dried over MgSO₄, filtered and evaporated under reduced pressure to afford 1-(1-(fluoromethyl)-2-oxabicyclo[2.1.1]hexan-4-yl)ethan-1-one (700 mg, quantitative yield) as a yellow oil.

### Preparation 166: 2-Bromo-1-(1-(fluoromethyl)-2-oxabicyclo[2.1.1]hexan-4-yl)ethan-1-one

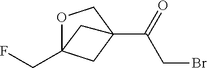 [SMILES: O=C(CBr)C12COC(CF)(C1)C2]

Copper(II) bromide (1.39 g, 6.20 mmol) was added to 1-(1-(fluoromethyl)-2-oxabicyclo[2.1.1]hexan-4-yl)ethan-1-one (Preparation 165, 700 mg, 4.43 mmol) in EtOH (12.66 mL) and the reaction stirred at 70° C. for 20 min. The reaction was quenched with ice and partitioned between EtOAc and water/brine and the layers separated. The aqueous phase was extracted with EtOAc (3×), the combined organic layers washed with NaHCO₃, brine, then dried over MgSO₄, filtered, and evaporated under reduced pressure to afford 2-bromo-1-(1-(fluoromethyl)-2-oxabicyclo[2.1.1]hexan-4-yl)ethan-1-one (900 mg, 85.7% yield) as a yellow oil.

### Preparation 167: 2-Bromo-1-(1-methyl-2-oxabicyclo[2.2.2]octan-4-yl)ethan-1-one

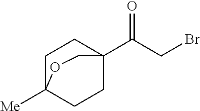 [SMILES: CC12CCC(C(=O)CBr)(CC1)CO2]

DMF (few drops), followed by oxalyl dichloride (5.43 mL, 63 mmol) were added dropwise to a solution of 1-methyl-2-oxabicyclo[2.2.2]octane-4-carboxylic acid (7.15 g, 42 mmol) in DCM (150 mL) under Ar and the reaction stirred at rt overnight. The mixture was evaporated under reduced pressure to provide 1-methyl-2-oxabicyclo[2.2.2]octane-4-carbonyl chloride. This was dissolved in DCM (50 mL), cooled to 0° C., ethereal diazomethane (3 eq. in 1 L Et₂O) was added and the reaction stirred for 30 mins. A stream of Ar was passed through the solution to remove excess diazomethane and the solution evaporated under reduced pressure. The crude was purified by silica gel column chromatography eluting with (EtOAc:Hex 30:70%) to provide 3-diazo-1-(1-methyl-2-oxabicyclo[2.2.2]octan-4-yl)propan-1-one, 5.0 g. This product (5.0 g, 26 mmol) was dissolved in DCM (200 mL), the solution cooled to 0° C., excess 40% aq HBr added and the reaction stirred for 1 h. The layers were separated, the organic layer was washed with sat. Na₂CO₃ soln. and dried over Na₂SO₄. The filtrate was evaporated under reduced pressure to afford 2-bromo-1-(1-methyl-2-oxabicyclo[2.2.2]octan-4-yl)ethan-1-one (5.3 g, 83%) as a brown crystalline powder.

### Preparation 168: 1-(2-Oxabicyclo[2.1.1]hexan-4-yl)-2-bromoethan-1-one

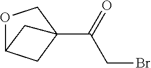

was obtained as a yellow solid, 2.70 g, 83% yield, from 2-oxabicyclo[2.1.1]hexane-4-carboxylic acid, following the procedure described in Preparation 167.

### Preparation 169: 2-Bromo-1-(1,3,3-trimethyl-2-oxabicyclo[2.1.1]hexan-4-yl)ethan-1-one

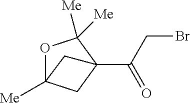 [SMILES: CC12CC(C(=O)CBr)(C1)C(C)(C)O2]

was obtained as a yellow crystalline powder from 1,3,3-trimethyl-2-oxabicyclo[2.1.1]hexane-4-carboxylic acid, following the procedure described in Preparation 167.

### Preparation 170: 1-(2-Oxabicyclo[2.2.1]heptan-4-yl)-2-bromoethan-1-one

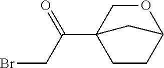 [SMILES: O=C(CBr)C12CCC(C1)OC2]

was obtained as a yellow oil, 5.2 g, from 2-oxabicyclo[2.2.1]heptane-4-carboxylic acid, following a similar procedure to that described in Preparation 167.

### Preparation 171: 2-Bromo-1-(1-methyl-2-oxabicyclo[2.2.1]heptan-4-yl)ethan-1-one

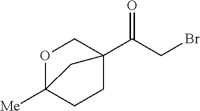 [SMILES: CC12CCC(C(=O)CBr)(CO1)C2]

was obtained as a yellow oil, 5.0 g, from 1-methyl-2-oxabicyclo[2.2.1]heptane-4-carboxylic acid following a similar procedure to that described in Preparation 167.

### Preparation 172: 3-(2-Bromoacetyl)bicyclo[1.1.1]pentane-1-carbonitrile

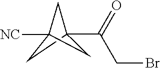 [SMILES: N#CC12CC(C(=O)CBr)(C1)C2]

To a solution of 1-cyanobicyclo[1.1.1]pentane-3-carboxylic acid (200 mg, 1.46 mmol) in DCM (10 mL) was added oxalyl dichloride (370.6 mg, 2.92 mmol) and the reaction stirred at 25° C. for 2 h. The mixture was concentrated in vacuo, the residue was dissolved in a mixture of MeCN (5 mL) and HBr (738.3 mg, 4.38 mmol, 48% purity) and diazomethyl(trimethyl)silane (2 M, 1.10 mL) added at 0° C. The reaction was stirred at 0° C. for 1 h and additional HBr (738.3 mg, 4.38 mmol, 48% purity) was added. The reaction was stirred at 0° C. for 30 mins, then the mixture was basified with aqueous NaHCO₃ to pH>7. The mixture was diluted with EtOAc (20 mL) and washed with water (10 mL×2). The organic layer was dried over Na₂SO₄, filtered and evaporated under reduced pressure to afford 3-(2-bromoacetyl)bicyclo[1.1.1]pentane-1-carbonitrile (250 mg, 72.0% yield) as yellow liquid. ¹HNMR (500 MHz, CDCl₃) δ: 1.58 (s, 2H), 2.61 (s, 2H), 3.89 (s, 2H), 4.13 (s, 2H).

### Preparation 173: 2-Bromo-1-(1-methoxycyclopropyl)ethan-1-one

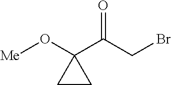

To a solution of 1-methoxycyclopropane-1-carboxylic acid (1.20 g, 10.34 mmol) in DCM (15 mL) was added SOCl₂ (2.46 g, 20.68 mmol) and DMF (1 drop), the reaction stirred at 20° C. for 1 h then concentrated in vacuo. The residue was diluted with MeCN (10 mL), THF (20 mL), the solution cooled to 0° C. and TMSCHN₂ (2 M, 10.34 mL) added. The mixture was stirred at 0° C. for 30 min, HBr (3.49 g, 20.68 mmol, 48% purity) added and the reaction stirred for a further 30 min. The reaction was quenched with saturated NaHCO₃aq. (30 mL) and extracted with EtOAc (30 mL×3). The combined organic extracts were washed with brine (30 mL), dried over Na₂SO₄ and filtered. The filtrate was evaporated under reduced pressure to afford 2-bromo-1-(1-methoxycyclopropyl)ethan-1-one (1.0 g, 50.1% yield) as a yellow oil. ¹H NMR (400 MHz, CDCl₃) δ: 1.28-1.31 (m, 2H), 1.39-1.41 (m, 2H), 3.41 (s, 3H), 4.34 (s, 2H)

### Preparation 174: 4-(Cyclopropylmethoxy)pyrimidin-2-amine

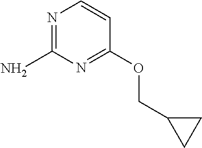 [SMILES: Nc1nccc(OCC2CC2)n1]

To a solution of cyclopropanemethanol (16.70 g, 231.6 mmol) in THF (100 mL) was added NaH (2.78 g, 69.48 mmol, 60% purity) at 0° C. under N₂ and the mixture stirred at 0° C. for 30 min. To the reaction mixture was added 4-chloropyrimidin-2-amine (3.0 g, 23.16 mmol), the reaction warmed to 15° C. and stirred for 14 h. The reaction mixture was quenched with water (50 mL), concentrated in vacuo and the residue extracted with EtOAc (100 mL×3). The combined organic layers were washed with brine (100 mL), dried over Na₂SO₄, filtered and concentrated. The residue was purified by silica gel chromatography (PE: EtOAc=1:1) to afford 4-(cyclopropylmethoxy)pyrimidin-2-amine (2.80 g, 69.5% yield) as yellow oil. ¹H NMR (500 MHz, CDCl₃) δ: 0.28-0.34 (m, 2H), 0.53-0.65 (m, 2H), 1.18-1.23 (m, 1H), 4.06 (d, 2H), 5.15 (br s, 2H), 6.07 (d, 1H), 7.98 (d, 1H)

### Preparation 175: 4-Cyclobutoxypyrimidin-2-amine

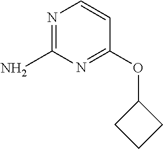 [SMILES: Nc1nccc(OC2CCC2)n1]

was obtained as a white solid, 3.83 g, 90.11% yield, from 4-chloropyrimidin-2-amine and cyclobutanol, following the procedure described in Preparation 174. LCMS m/z=166.0 [M+H]⁺

### Preparation 176: 2-Chloro-4-cyclobutoxy-3-fluoropyridine

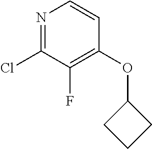 [SMILES: Fc1c(OC2CCC2)ccnc1Cl]

To a solution of 2-chloro-4-hydroxy-3-fluoropyridine (2.0 g, 13.56 mmol) in DMF (20 mL) was added K₂CO₃ (5.62 g, 40.68 mmol) and the reaction stirred at 25° C. for 2 h. Cyclobutanol (2.75 g, 20.34 mmol) was added and the reaction stirred at 60° C. for 16 h. The cooled reaction mixture was concentrated in vacuo and the residue purified by column chromatography on silica gel eluting with PE/EtOAc (3/1) to afford 2-chloro-4-cyclobutoxy-3-fluoropyridine (2.30 g, 71.5% yield) as a white solid. LCMS m/z=202.2 [M+H]⁺

### Preparation 177: 3-Fluoro-4-isopropoxypyridin-2-amine

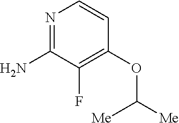 [SMILES: CC(C)Oc1ccnc(N)c1F]

A mixture of 2-chloro-3-fluoro-4-(propan-2-yloxy)pyridine (3.90 g, 20.57 mmol), t-butyl carbamate (3.37 g, 28.8 mmol), XantPhos-Pd-G3 (975.4 mg, 1.03 mmol) and Cs₂CO₃ (13.40 g, 41.14 mmol) were degassed in toluene (102.9 mL) and the reaction stirred at 90° C. overnight. The cooled mixture was diluted with water, extracted with EtOAc and the combined organic extracts concentrated in vacuo. The crude was purified by column chromatography on silica gel eluting with EtOAc/heptanes (0/100 to 100/0) to afford 3-fluoro-4-isopropoxypyridin-2-amine. LCMS m/z=171.0 [M+H]⁺

### Preparation 178: 4-Cyclobutoxy-3-fluoropyridin-2-amine

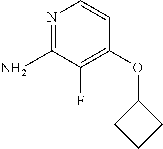 [SMILES: Nc1nccc(OC2CCC2)c1F]

Pd₂(dba)₃ (249.8 mg, 0.273 mmol), Xantphos (315.7 mg, 0.546 mmol) and Cs₂CO₃ (2.67 g, 8.18 mmol) were added to a solution of 2-chloro-4-cyclobutoxy-3-fluoropyridine (Preparation 176, 550 mg, 2.73 mmol) and diphenylmethanimine (1.48 g, 8.18 mmol) in toluene (20 mL), the mixture purged with N₂ then stirred at 110° C. for 12 h. The cooled mixture was concentrated in vacuo and the residue purified by column chromatography on silica gel (PE/EtOAc=3/1) to give N-(4-cyclobutoxy-3-fluoropyridin-2-yl)-1,1-diphenylmethanimine (880 mg, 79.1% yield) as a white solid. A solution of this compound in EtOAc/HCl (4M, 20 mL) was stirred at 20° C. for 16 h and the mixture concentrated in vacuo. The residue was neutralized using aq. NaHCO₃ (10 mL) and extracted with EtOAc (20 mL×3). The combined organic layers were washed with brine (20 mL), dried over Na₂SO₄, filtered and concentrated in vacuo. The residue was purified by column chromatography on silica gel eluting with PE/EtOAc (5/1 to 1/1) to afford 4-cyclobutoxy-3-fluoropyridin-2-amine (430 mg, 83.6% yield) as a yellow solid. LCMS m/z=183.0 [M+H]⁺

### Preparation 179: Isopropyl 4,6-dichloronicotinate

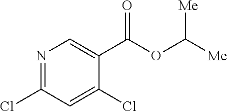 [SMILES: CC(C)OC(=O)c1cnc(Cl)cc1Cl]

To a mixture of 4,6-dichloronicotinoyl chloride (5.48 g, 26.04 mmol) in THF (50 mL) was added TEA (3.95 g, 39.06 mmol) and propan-2-ol (2.35 g, 39.06 mmol) and the reaction stirred at 20° C. for 1 h. The mixture was diluted with water (50 mL), extracted with EtOAc (50 mL×3) and the combined organic extracts washed with brine and dried over Na₂SO₄. The mixture was filtered, the filtrate concentrated in vacuo and the residue purified by silica gel chromatography (PE: EtOAc=5/1) to give isopropyl 4,6-dichloronicotinate (4.70 g, 73.2% yield) as a yellow liquid. LCMS m/z=233.9 [M+H]⁺

### Preparation 180: Isopropyl 6-chloro-4-isopropoxynicotinate

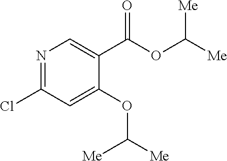 [SMILES: CC(C)OC(=O)c1cnc(Cl)cc1OC(C)C]

NaH (481.9 mg, 60%, 20.1 mmol) was added to propan-2-ol (45.82 mL, 602.4 mmol) at 0° C. and the solution stirred at 0° C. for 1 h. A solution of isopropyl 4,6-dichloronicotinate (Preparation 179, 4.70 g, 20.1 mmol) in THF (50 mL) was added and the reaction stirred at 10-15° C. for 16 h. The reaction was quenched with water (100 mL) and extracted with EtOAc (100 mL×3). The combined organic layers were washed with brine (30 mL), dried over Na₂SO₄ and filtered. The filtrate was concentrated in vacuo and the residue purified by Combiflash® (PE/EtOAc=3/1) to afford isopropyl 6-chloro-4-isopropoxynicotinate (3.00 g, 52.2% yield) as colorless oil. LCMS m/z=257.9 [M+H]⁺

### Preparation 181: Isopropyl 6-((tert-butoxycarbonyl)amino)-4-isopropoxynicotinate

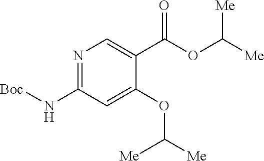 [SMILES: CC(C)OC(=O)c1cnc(NC(=O)OC(C)(C)C)cc1OC(C)C]

To a solution of isopropyl 6-chloro-4-isopropoxynicotinate (Preparation 180, 1.50 g, 5.82 mmol) and tert-butyl carbamate (818.3 mg, 6.98 mmol) in toluene (30 mL) was added Pd₂(dba)₃ (266.5 mg, 0.291 mmol), Xantphos (336.8 mg, 0.582 mmol) and Cs₂CO₃ (3.79 g, 11.64 mmol), the mixture degassed with N₂ and the reaction stirred at 100° C. for 16 h. The cooled mixture was concentrated in vacuo and the residue purified by Combiflash® (PE/EtOAc=10/1 to 1/1) to afford isopropyl 6-((tert-butoxycarbonyl)amino)-4-isopropoxynicotinate (3.60 g, crude) as yellow oil. ¹HNMR (500 MHz, CDCl₃) δ: 1.34 (d, 6H), 1.43 (d, 6H), 1.55 (s, 9H), 4.79-4.84 (m, 1H), 5.21-5.27 (m, 1H), 7.66 (s, 1H), 8.64 (s, 1H), 8.75 (br s, 1H).

### Preparation 182: Isopropyl 6-amino-4-isopropoxynicotinate hydrochloride

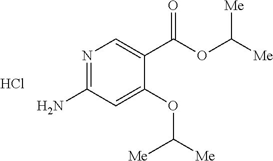 [SMILES: Cl ; CC(C)OC(=O)c1cnc(N)cc1OC(C)C]

To a solution of isopropyl 6-((tert-butoxycarbonyl)amino)-4-isopropoxynicotinate (Preparation 181, 3.60 g, 10.64 mmol) was added HCl/EtOAc (20 mL) and the reaction stirred at 20° C. for 16 h. The mixture was evaporated under reduced pressure to afford isopropyl 6-amino-4-isopropoxynicotinate hydrochloride (3.0 g, crude) as yellow oil. ¹HNMR (500 MHz, MeOH-d₄) δ: 1.33 (d, 6H), 1.43 (d, 6H), 4.80-4.82 (m, 1H), 5.14-5.19 (m, 1H), 6.40 (s, 1H), 8.28 (s, 1H).

### Preparation 183: Isopropyl 6-amino-5-chloro-4-isopropoxynicotinate

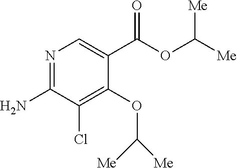 [SMILES: CC(C)OC(=O)c1cnc(N)c(Cl)c1OC(C)C]

To a solution of isopropyl 6-amino-4-isopropoxynicotinate hydrochloride (Preparation 182, 500 mg, 2.10 mmol) in MeCN (10 mL) was added NCS (280.4 mg, 2.10 mmol) and the reaction stirred at 25° C. for 16 h. The mixture was diluted with water (100 mL) and extracted with EtOAc (100 mL×3). The combined organic extracts were washed with Na₂SO₃ aq. (50 mL), dried over Na₂SO₄ and filtered. The filtrate was concentrated in vacuo and the residue purified by Combiflash® (PE/EtOAc=3/1) to afford isopropyl 6-amino-5-chloro-4-isopropoxynicotinate, 240 mg, 37.7% yield, as brown oil. LCMS m/z=273.1 [M+H]⁺

### Preparation 184: 5-Bromo-4-(difluoromethoxy)pyridin-2-amine

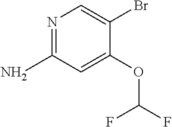 [SMILES: Nc1cc(OC(F)F)c(Br)cn1]

1-Bromopyrrolidine-2,5-dione (1.11 g, 6.25 mmol) was added to a mixture of 4-(difluoromethoxy)pyridin-2-amine (1.00 g, 6.25 mmol) in MeCN (15.63 mL) at 0° C. and the reaction stirred at rt for 2 h. The reaction was quenched with aq. sat. NaHCO₃, extracted with EtOAc (3×), the combined organic extracts dried over MgSO₄, filtered and concentrated in vacuo. The crude product was purified by column chromatography on silica gel eluting with heptanes/(3:1 EtOAc:EtOH) (100/0 to 50/50), to afford 5-bromo-4-(difluoromethoxy)pyridin-2-amine (1.10 g, 73.6% yield). LCMS m/z=238.9 [M+H]⁺

### Preparations 185 to 192

The compounds in the following table were prepared from the appropriate amine (RNH₂) and 1-bromopyrrolidine-2,5-dione, following the procedure described in Preparation 184.

| Prep no | Structure/Name/RNH₂/Yield/Data |
|---|---|
| 185 | 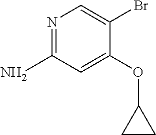 [SMILES: Nc1cc(OC2CC2)c(Br)cn1] |
| | 5-bromo-4-cyclopropoxypyridin-2-amine, RNH₂: 4-cyclopropoxypyridin-2- |
| | amine 900 mg, 88.2% yield. LCMS m/z = 229.0 [M + H]⁺ |
| 186 | 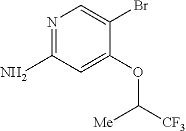 [SMILES: CC(Oc1cc(N)ncc1Br)C(F)(F)F] |
| | 5-bromo-4-((1,1,1-trifluoropropan-2-yl)oxy)pyridin-2-amine |
| | RNH₂: 4-((1,1,1-trifluoropropan-2-yl)oxy)pyridin-2-amine |
| | 850 mg, 87.7% yield LCMS m/z = 284.9 [M + H]⁺ |
| 187 | 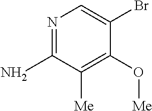 [SMILES: COc1c(Br)cnc(N)c1C] |
| | 5-bromo-4-methoxy-3-methylpyridin-2-amine, RNH₂: 4-methoxy-3- |
| | methylpyridin-2-amine 1.26 g, crude. LCMS m/z = 217.1 [M + H]⁺ |
| 188 | 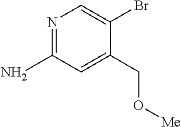 [SMILES: COCc1cc(N)ncc1Br] |
| | 5-bromo-4-(methoxymethyl)pyridin-2-amine, RNH₂: 4- |
| | (methoxymethyl)pyridine-2-amine 1.20 g, 76.4% yield. LCMS m/z = 217.0 |
| | [M + H]⁺ |
| 189 | 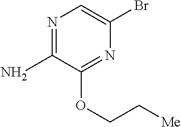 [SMILES: CCCOc1nc(Br)cnc1N] |
| | 5-bromo-3-propoxypyrazin-2-amine, RNH₂: 3-propoxypyrazin-2-amine |
| | 620.8 mg, 40.9% yield as an orange solid. LCMS m/z = 232.1, 234.1 [M + H]⁺ |
| 190 | 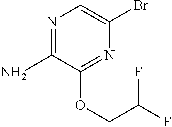 [SMILES: Nc1ncc(Br)nc1OCC(F)F] |
| | 5-bromo-3-(2,2-difluoroethoxy)pyrazin-2-amine, RNH₂: 3-(2,2- |
| | difluoroethoxy)pyrazin-2-amine 1.0 g, 68.9% yield. LCMS m/z = 256.0 [M + H]⁺ |
| 191 | 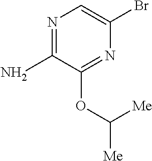 [SMILES: CC(C)Oc1nc(Br)cnc1N] |
| | 5-bromo-3-isopropoxypyrazin-2-amine, RNH₂: 3-isopropoxypyrazin-2-amine |
| | 1.40 g, 36.9% yield. LCMS m/z = 234.1 [M + H]⁺ |
| 192 | 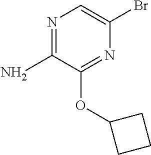 [SMILES: Nc1ncc(Br)nc1OC1CCC1] |
| | 5-bromo-3-cyclobutoxypyrazin-2-amine |
| | RNH₂: 3-(cyclobutoxy)pyrazin-2-amine 950 mg, 64.3% yield |

### Preparation 193: 5-Bromo-4-(methoxymethyl)pyrimidin-2-amine

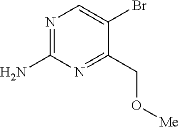 [SMILES: COCc1nc(N)ncc1Br]

1-Bromopyrrolidine-2,5-dione (1.28 g, 7.19 mmol) was added to a mixture of 4-(methoxymethyl)pyrimidin-2-amine (1.00 g, 7.19 mmol) in MeCN (10.34 mL) at 0° C. and the reaction stirred at rt for 2 h. The resulting suspension was filtered and the solid dried in vacuo to afford 5-bromo-4-(methoxymethyl)pyrimidin-2-amine (1.10 g, 70.1% yield) as a white solid. LCMS m/z=218.0 [M+H]⁺

### Preparation 194: 5-Bromo-3-fluoro-4-isopropoxypyridin-2-amine

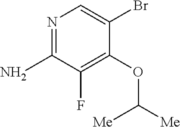 [SMILES: CC(C)Oc1c(Br)cnc(N)c1F]

3-Fluoro-4-isopropoxypyridin-2-amine (Preparation 177, 1.10 g, 6.46 mmol) and S (1.15 g, 6.46 mmol) were stirred in MeCN (64.6 mL) at rt for 1 h. The reaction was diluted with water, extracted with EtOAc, the combined organic extracts dried over Na₂SO₄ and then concentrated in vacuo. The crude was purified by column chromatography on silica gel eluting with Et₀H/EtOAc (0/100 to 10/90) to afford 5-bromo-3-fluoro-4-isopropoxypyridin-2-amine. LCMS m/z=248.9 [M+H]⁺

### Preparation 195: 5-Bromo-4-cyclobutoxy-3-fluoropyridin-2-amine

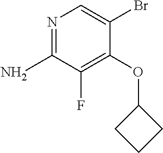 [SMILES: Nc1ncc(Br)c(OC2CCC2)c1F]

was obtained as a yellow solid, from 4-cyclobutoxy-3-fluoropyridin-2-amine (Preparation 178) following a similar procedure to that described in Preparation 194, 430 mg, 59.3% yield. LCMS m/z=263.1 [M+H]⁺

### Preparation 196: 5-Iodo-4-isopropoxypyrimidin-2-amine

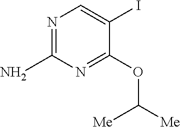 [SMILES: CC(C)Oc1nc(N)ncc1I]

To a solution of 4-isopropoxypyrimidin-2-amine (9.60 g, 62.67 mmol) in DCM (200 mL) was added NIS (14.10 g, 62.67 mmol) at 0° C. and the reaction stirred at 15° C. for 14 h. The mixture was quenched with saturated aq. Na₂SO₃ (150 mL) and the layers separated. The organic layer was washed with brine (100 mL×2), dried over Na₂SO₄, filtered and concentrated in vacuo. The residue was purified by Combiflash® (PE/EtOAc=1/1) to afford 5-iodo-4-isopropoxypyrimidin-2-amine (9.10 g, 49.4% yield) as a yellow solid. ¹HNMR (500 MHz, CDCl₃) δ:1.37 (d, 6H), 4.87 (br s, 2H), 5.28-5.31 (m, 1H), 8.24 (s, 1H).

### Preparation 197: 4-Cyclobutoxy-5-iodopyrimidin-2-amine

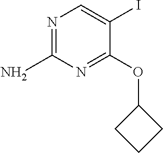 [SMILES: Nc1ncc(I)c(OC2CCC2)n1]

To a solution of 4-cyclobutoxypyrimidin-2-amine (Preparation 175, 4.20 g, 25.43 mmol) in DCM (100 mL) was added NIS (5.72 g, 25.43 mmol) at 0° C. under N₂ and the reaction stirred at 25° C. for 16 h. The mixture was quenched with saturated Na₂SO₃ aq. (200 mL) and the layers separated. The organic layer was washed with brine (200 mL) and dried over Na₂SO₄, filtered and concentrated in vacuo. The residue was purified by Combiflash® eluting with PE/EtOAc (0 to 1/1) to afford 4-cyclobutoxy-5-iodopyrimidin-2-amine (5.50 g, 66.8% yield) as a yellow solid. LCMS m/z=292.5 [M+H]⁺

### Preparation 198: 4-(Cyclopropylmethoxy)-5-iodopyrimidin-2-amine

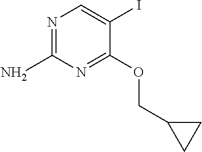 [SMILES: Nc1ncc(I)c(OCC2CC2)n1]

To a solution of 4-(cyclopropylmethoxy)pyrimidin-2-amine (Preparation 174, 2.80 g, 16.95 mmol) in DCM (100 mL) was added 1-iodopyrrolidine-2,5-dione (7.63 g, 33.9 mmol) at 0° C. under N₂ and the reaction stirred at 25° C. for 16 h. The mixture was quenched with saturated aq. Na₂SO₃ (50 mL) and extracted with EtOAc (100 mL). The organic layers were washed with brine (100 mL×2), dried over Na₂SO₄, filtered and concentrated in vacuo. The residue was purified by Combiflash® eluting with PE/EtOAc (1/1) to afford 4-(cyclopropylmethoxy)-5-iodopyrimidin-2-amine (2.90 g, 55.8% yield) as yellow solid. LCMS m/z=291.8 [M+H]⁺

### Preparation 199: 4-(Benzyloxy)-5-bromopyridin-2-amine

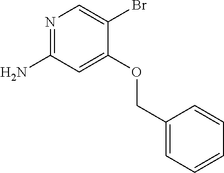 [SMILES: Nc1cc(OCc2ccccc2)c(Br)cn1]

NaH (28.92 g, 0.723 mmol, 60% purity) was added to a suspension of 5-bromo-4-chloropyridin-2-amine (100 g, 0.482 mmol) in DMF (800 mL) at −5° C. under Ar and the mixture stirred for 30 min. Benzyl alcohol (78.19 g, 0.723 mmol) was added dropwise at 0° C. and the resulting mixture stirred at rt under Ar for 48 h. The mixture was diluted with H₂O (1000 mL), extracted with EtOAc (3×250 mL) and the combined organics washed with brine, dried (Na₂SO₄) and evaporated to dryness in vacuo. The residue was crystallized from hexane:DCM (600 mL:200 mL) and the precipitate collected by filtration, washed with hexane and air-dried to give 4-(benzyloxy)-5-bromopyridin-2-amine as a yellow solid (87 g, 62%).

### Preparation 199A: 4-(Benzyloxy)-5-bromopyridin-2-amine hydrobromide

4-(Benzyloxy)pyridin-2-amine (18.2 g, 90.9 mmol) was suspended in AcOH (70 mL) under N₂, the mixture was cooled on an ice-water bath, bromine (4.69 mL, 90.9 mmol) was added slowly over a period of 10 min and the reaction stirred at rt for 10 mins. The resulting precipitate was filtered off and washed with AcOH. The solids were dried in vacuo then stirred in a mixture of DCM (100 mL) and MeOH (2.5 mL) for 4 h. The solids were collected by filtration, washed with DCM (2×5 mL) and dried in vacuo to afford 4-(benzyloxy)-5-bromopyridin-2-amine hydrobromide (22.7 g, 69%) as an off-white solid.

### Preparation 200: 5-Bromo-3-isopropoxypyridin-2-amine

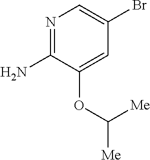 [SMILES: CC(C)Oc1cc(Br)cnc1N]

To a solution of 2-amino-5-bromopyridin-3-ol (1.00 g, 5.29 mmol) in DCM (15 mL) was added 2-iodopropane (1.80 g, 10.58 mmol) and 40% NaOH solution (10 mL). N-methyl-N,N-dioctyloctan-1-aminium chloride (373.2 mg, 1.06 mmol) was added and the reaction stirred at 25° C. for 16 h. The reaction was concentrated in vacuo and the residue partitioned between EtOAc (30 mL) and water (30 mL) and the layers separated. The organic phase was evaporated under reduced pressure and the crude was purified by silica gel column chromatography eluting with PE/EtOAc, (86/14) to afford 5-bromo-3-isopropoxypyridin-2-amine, (460 mg, 35.75% yield) as white solid. ¹H NMR (400 MHz, DMSO-d₆) δ: 1.26 (d, 6H), 4.57-4.63 (m, 1H), 5.82 (s, 2H), 7.20 (d, 1H), 7.56 (d, 1H)

### Preparation 201: 5-Bromo-4-cyclopropoxypyrimidin-2-amine

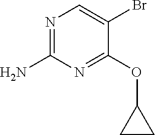 [SMILES: Nc1ncc(Br)c(OC2CC2)n1]

Cyclopropanol (1.67 g, 28.79 mmol) and Cs₂CO₃ (12.51 g, 38.38 mmol) were added to a solution of 5-bromo-4-chloropyrimidin-2-amine (4.0 g, 19.19 mmol) in DMF (48.0 mL) and the reaction heated at 70° C. for 2 h. The cooled solution was diluted with brine, extracted with EtOAc, the combined organic extracts dried and concentrated in vacuo. The residue was purified by column chromatography on silica gel to afford 5-bromo-4-cyclopropoxypyrimidin-2-amine (2.0 g, 45.3% yield). LCMS m/z=231.9 [M+H]⁺

### Preparation 202: tert-Butyl (5-bromo-3-fluoro-4-isopropoxypyridin-2-yl)carbamate

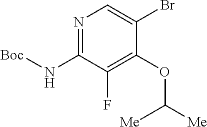 [SMILES: CC(C)Oc1c(Br)cnc(NC(=O)OC(C)(C)C)c1F]

(Boc)₂O (482.5 mg, 2.21 mmol) and DMAP (24.5 mg, 0.20 mmol) were added to a solution of 5-bromo-3-fluoro-4-isopropoxypyridin-2-amine (Preparation 194, 500.6 mg, 2.01 mmol) in DCM (20.1 mL) and the reaction stirred at rt for 2 h. The reaction was diluted with water and extracted with EtOAc. The combined organic extracts were dried over Na₂SO₄ and evaporated under reduced pressure to afford tert-butyl (5-bromo-3-fluoro-4-isopropoxypyridin-2-yl)carbamate. LCMS m/z=294.8 [M-tBu+H]⁺

### Preparation 203: Phenyl 6-amino-5-fluoro-4-isopropoxynicotinate

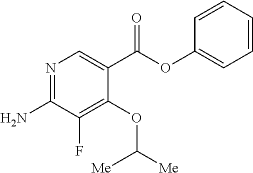 [SMILES: CC(C)Oc1c(C(=O)Oc2ccccc2)cnc(N)c1F]

Phenyl formate (158.6 mg, 1.30 mmol), XantPhos-Pd-G3 (67.2 mg, 0.065 mmol) and TEA (131.5 mg, 1.30 mmol) were added sequentially to a solution of tert-butyl (5-bromo-3-fluoro-4-isopropoxypyridin-2-yl)carbamate (Preparation 202, 370 mg, 1.06 mmol) in MeCN (6.49 mL), the flask purged with N₂, sealed and heated at 80° C. for 2 h. The cooled reaction was diluted with water and extracted with EtOAc. The combined organic extracts were washed with brine, dried over Na₂SO₄ and concentrated in vacuo. The crude was purified by column chromatography on silica gel eluting with 0 to 100/0 EtOAc-heptanes to afford phenyl 6-amino-5-fluoro-4-isopropoxynicotinate. LCMS m/z=290.0 [M+H]⁺

### Preparation 204: Methyl 6-amino-4-cyclobutoxynicotinate

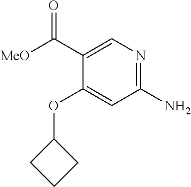 [SMILES: COC(=O)c1cnc(N)cc1OC1CCC1]

Part A: NaH (60% dispersion in mineral oil, 39.15 g, 979 mmol) was added in portions to a solution of cyclobutanol (70.47 g, 979 mmol) in DMF (1 L) at rt and stirred until H₂ evolution ceased. 5-Bromo-4-chloropyridin-2-amine (193.4 g, 932 mmol) was added and the resulting solution stirred at 100° C. for 24 h. The cooled reaction mixture was diluted with water (4 L) and extracted with EtOAc (2×500 mL). The combined organics were washed with H₂O (4×300 mL), dried (Na₂SO₄) and evaporated to dryness in vacuo. The solid residue was crystallized from benzene to give 5-bromo-4-cyclobutoxypyridin-2-amine (142.7 g, 63%).

Part B. 5-Bromo-4-cyclobutoxypyridin-2-amine (142.7 g, 587 mmol), TEA (65.2 g, 646 mmol) and Pd(dppf)Cl₂.DCM (14.38 g, 17.6 mmol) were dissolved in dry MeOH (800 mL) and the reaction heated to 140° C. at 40 bar of CO for 12 h. The cooled mixture was concentrated in vacuo, the mixture poured into water (1 L) and extracted with EtOAc (3×200 mL). The combined organics were dried (Na₂SO₄) and evaporated to dryness in vacuo. The residue was recrystallised from isopropanol to afford methyl 6-amino-4-cyclobutoxynicotinate (79.5 g, 61%).

### Preparation 205: Methyl 6-amino-4-(benzyloxy)nicotinate

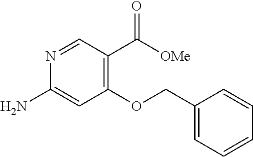 [SMILES: COC(=O)c1cnc(N)cc1OCc1ccccc1]

was obtained from 4-benzyloxy-5-bromopyridin-2-amine, 48 g, 59%, following a similar procedure to that described in Preparation 204, except the compound was crystallized from DCM:hexane (1:3 V/V). LCMS m/z=259.2 [M+H]⁺

### Preparation 206: Isopropyl 2-amino-4-isopropoxypyrimidine-5-carboxylate

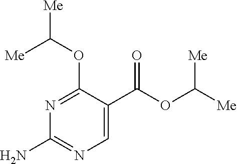 [SMILES: CC(C)OC(=O)c1cnc(N)nc1OC(C)C]

Part A: To a suspension of ethyl 2-amino-6-oxo-1,6-dihydropyrimidine-5-carboxylate (71 g, 387 mmol) in AcOH (1.5 L) was added acetic anhydride (73 mL, 773 mmol) and the reaction stirredat reflux for 18 h. The cooled mixture was filtered and the solid washed with hexane and dried at 60° C. for 24 h to afford ethyl 2-acetamido-6-oxo-1,6-dihydropyrimidine-5-carboxylate (80 g, 92% yield).

Part B: Ethyl 2-acetamido-6-oxo-1,6-dihydropyrimidine-5-carboxylate (80 g, 356 mmol) was dissolved in POCl₃ (800 mL) and the reaction mixture heated at 60° C. for 16 h. Excess POCl₃ was removed in vacuo and the residue poured into ice. The mixture was extracted with DCM and the combined organic layers were dried over Na₂SO₄, filtered and the filtrate evaporated under reduced pressure to provide ethyl 2-acetamido-4-chloropyrimidine-5-carboxylate, 87 g.

Part C: To a solution of Na (9.9 g, 420 mmol) in isopropanol (1.5 L) was added portion wise, ethyl 2-acetamido-4-chloropyrimidine-5-carboxylate (30 g, 123 mmol) and the reaction stirred for 12 h at rt. The mixture was concentrated in vacuo, the residue dissolved in water, and extracted with EtOAc. The combined organic layers were dried over Na₂SO₄ and evaporated under reduced pressure. The crude residue was crystallized from EtOAc/hexane to afford isopropyl 2-amino-4-isopropoxypyrimidine-5-carboxylate, 4.4 g.

### Preparation 207: Ethyl 2-amino-4-ethoxypyrimidine-5-carboxylate

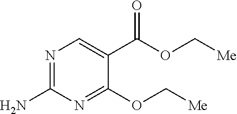 [SMILES: CCOC(=O)c1cnc(N)nc1OCC]

was prepared from ethyl 2-acetamido-4-chloropyrimidine-5-carboxylate (Preparation 206, Part B) and EtOH, following the procedure described in Preparation 206, 13.9 g, 55% yield.

### Preparation 208: Methyl 5-amino-6-ethoxypyrazine-2-carboxylate

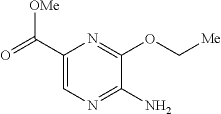 [SMILES: CCOc1nc(C(=O)OC)cnc1N]

Part A: A solution of 3,5-dibromopyrazin-2-amine (47.4 g, 190 mmol) and NaOEt (14 g, 206 mmol) in EtOH (500 mL) was heated at reflux for 8 h. The reaction mixture was evaporated to dryness in vacuo and the residue partitioned between H₂O (400 mL) and EtOAc (500 mL). The combined organics were dried (MgSO₄) and evaporated to dryness in vacuo to afford 5-bromo-3-ethoxypyrazin-2-amine (36.8 g, 90%).

Part B. 5-Bromo-3-ethoxypyrazin-2-amine (36.8 g, 169 mmol), PdCl₂(dppf)₂ (0.7 g) and TEA (27.6 mL, 200 mmol) were added to MeOH (600 mL) in an autoclave. The reactor was charged with 40 bar CO (gas) and heated at 100° C. overnight. The cooled reaction mixture was concentrated in vacuo and the residue partitioned between EtOAc and H₂O and the layers separated. The combined organics were washed with brine, dried (Na₂SO₄) and evaporated to dryness. The residue was purified by silica gel column chromatography to give methyl 5-amino-6-ethoxypyrazine-2-carboxylate (25 g, 75%). LCMS m/z=198.0 [M+H]⁺.

### Preparation 209: 3-(Benzyloxy)-5-bromopyrazin-2-amine

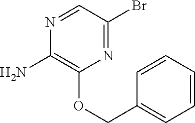 [SMILES: Nc1ncc(Br)nc1OCc1ccccc1]

A suspension of NaH (6.31 g, 158 mmol) in dry THF (500 mL) at 0° C. was stirred for 10 mins, then benzyl alcohol (16.4 mL, 158 mmol) was added and the solution stirred for 30 mins. 3,5-Dibromopyrazin-2-amine (26.6 g, 105 mmol) was added and the reaction was warmed to reflux and stirred for 10 h. The cooled mixture was poured into ice water (1 L) and the aqueous solution extracted with EtOAc (3×500 mL). The combined organic phases were washed with brine (2×300 mL), dried over anhydrous Na₂SO₄, Filtered, and concentrated under reduced pressure. The crude residue was purified by silica gel chromatography to afford 3-(benzyloxy)-5-bromopyrazin-2-amine (25 g, 85% yield) as a yellow solid.

### Preparation 210: Methyl 5-amino-6-(benzyloxy)pyrazine-2-carboxylate

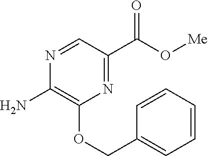 [SMILES: COC(=O)c1cnc(N)c(OCc2ccccc2)n1]

3-(Benzyloxy)-5-bromopyrazin-2-amine (Preparation 209, 34 g, 120 mmol), PdCl₂(dppf)₂ (0.7 g) and TEA (19.9 mL, 145 mmol) were added to MeOH (600 mL) in an autoclave. The reactor was charged with 40 bar CO (gas) and heated at 100° C. overnight. The reaction mixture was concentrated in vacuo and the residue partitioned between EtOAc and water. The layers were separated and the organic layer was washed with brine and dried over Na₂SO₄. The mixture was concentrated in vacuo and purified by silica gel column chromatography to afford methyl 5-amino-6-(benzyloxy)pyrazine-2-carboxylate (25 g, 80% yield).

### Preparation 211: Ethyl 2-amino-4-(cyclopentyloxy)pyrimidine-5-carboxylate

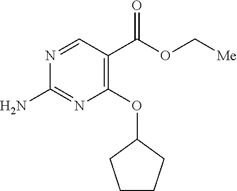 [SMILES: CCOC(=O)c1cnc(N)nc1OC1CCCC1]

NaH (97.9 mg, 2.45 mmol, 60% purity) was added portion wise to a mixture of cyclopentanol (210.8 mg, 2.45 mmol) in THF (9.32 mL) at 0° C. and the solution stirred for 30 min at rt. Ethyl 2-amino-4-chloropyrimidine-5-carboxylate (470 mg, 2.33 mmol) was added and the reaction stirred at rt. The reaction was quenched with aq. sat. NaCl, extracted with EtOAc (3×), the combined organic extracts dried over MgSO₄, filtered, and evaporated under reduced pressure to afford ethyl 2-amino-4-(cyclopentyloxy)pyrimidine-5-carboxylate. LCMS m/z=252.1 [M+H]⁺

### Preparation 212: 2-(5-Bromo-2-imino-4-isopropoxypyridin-1(2H)-yl)acetic acid

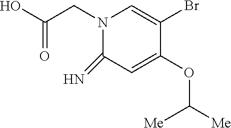 [SMILES: CC(C)Oc1cc(=N)n(CC(=O)O)cc1Br]

TEA (403.1 mg, 3.98 mmol) was added dropwise to a mixture of 2-chloroacetic acid (339.6 mg, 3.59 mmol) and water (1 mL) and the solution stirred for 10 mins. 5-Bromo-4-isopropoxypyridin-2-amine (Preparation 1, 1.0 g, 4.33 mmol) was added and the reaction stirred at 90° C. for 2 h. The reaction was cooled to 0° C., EtOH was added and the mixture stirred at 0° C. for 30 mins. The resulting mixture was filtered and the solid dried to afford 2-(5-bromo-2-imino-4-isopropoxypyridin-1(2H)-yl)acetic acid. LCMS m/z=290.0 [M+H]⁺

### Preparation 213: 6-Bromo-2-chloro-7-isopropoxyimidazo[1,2-a]pyridine

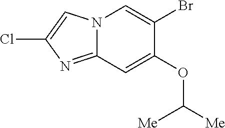 [SMILES: CC(C)Oc1cc2nc(Cl)cn2cc1Br]

Phosphorus(V) oxide chloride (594.9 mg, 3.88 mmol) was added to a suspension of 2-(5-bromo-2-imino-4-isopropoxypyridin-1(2H)-yl)acetic acid (Preparation 212, 560.9 mg, 1.94 mmol) in toluene (19.4 mL) and the reaction was warmed to 120° C. for 2 h under microwave irradiation. The cooled reaction was slowly poured into ice water and the mixture stirred for 10 mins. The phases were separated and the aqueous phase neutralised with 1N NaOH. This was extracted with EtOAc and the combined organic extracts dried over Na₂SO₄ and concentrated in vacuo. The crude was purified by column chromatography on silica gel eluting with EtOAc/heptanes (0/100 to 100/0) to afford 6-bromo-2-chloro-7-isopropoxyimidazo[1,2-a]pyridine. LCMS m/z=290.8 [M+H]⁺

### Preparation 214:7-Cyclobutoxy-6-iodo-2-(1-methyl-2-oxabicyclo[2.1.1]hexan-4-yl)imidazo[1,2-a]pyrimidine

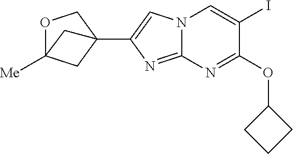 [SMILES: CC12CC(c3cn4cc(I)c(OC5CCC5)nc4n3)(CO1)C2]

To a solution of 4-cyclobutoxy-5-iodopyrimidin-2-amine (Preparation 197, 600 mg, 2.06 mmol) and 2-bromo-1-(1-methyl-2-oxabicyclo[2.1.1]hexan-4-yl)ethan-1-one (Preparation 36, 600 mg, 2.74 mmol) in t-BuOH (10 mL) was added NaHCO₃ (346.1 mg, 4.12 mmol) and the reaction stirred at 100° C. for 16 h. The mixture was concentrated in vacuo and the residue was purified by CombiFlash® eluting with PE/EtOAc (0 to 1/1) to afford 7-cyclobutoxy-6-iodo-2-(1-methyl-2-oxabicyclo[2.1.1]hexan-4-yl)imidazo[1,2-a]pyrimidine (431 mg, 50.9% yield) as a yellow solid. LCMS m/z=412.1 [M+H]⁺

### Preparations 215 to 219

The following compounds were prepared from the appropriate amine and halo ketone, following a similar procedure to that described in Preparation 214.

| Prep. No | Structure/Name/Starting Materials (SM)/Yield/Data |
|---|---|
| 215 | 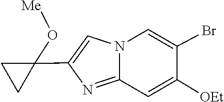 [SMILES: CCOc1cc2nc(C3(OC)CC3)cn2cc1Br] |
| | 6-bromo-7-ethoxy-2-(1-methoxycyclopropyl)imidazo[1,2-a]pyridine |
| | SM: 2-bromo-1-(1-methoxycyclopropyl)ethan-1-one (Preparation 173) and |
| | 5-bromo-4-ethoxypyridin-2-amine yellow oil, 600 mg, 52.3% yield |
| | ¹H NMR (400 MHz, CDCl₃) δ: 1.22 (s, 2H), 1.23 (s, 2H), 1.51 (t, 3H), 3.42 |
| | (s, 3H), 4.10-4.13 (m, 2H), 6.84 (s, 1H), 7.41 (s, 1H), 8.19 (s, 1H). |
| 216 | 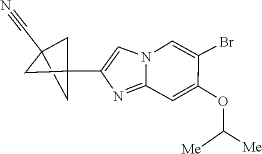 [SMILES: CC(C)Oc1cc2nc(C34CC(C#N)(C3)C4)cn2cc1Br] |
| | 3-(6-bromo-7-isopropoxyimidazo[1,2-a]pyridin-2-yl)bicyclo[1.1.1]pentane- |
| | 1-carbonitrile SM: 3-(2-bromoacetyl)bicyclo[1.1.1]pentane-1-carbonitrile |
| | (Preparation 172) and 5-bromo-4-isopropoxypyridin-2-amine (Preparation |
| | 1) yellow liquid 300 mg, 66.6% yield. LCMS m/z = 347.9 [M + H]⁺ |
| 217 | 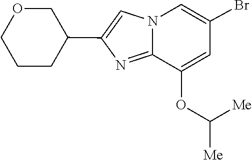 [SMILES: CC(C)Oc1cc(Br)cn2cc(C3CCCOC3)nc12] |
| | 6-bromo-8-isopropoxy-2-(tetrahydro-2H-pyran-3-yl)imidazo[1,2-a]pyridine |
| | SM: 2-bromo-1-(tetrahydro-2H-pyran-3-yl)ethan-1-one and 5-bromo-3- |
| | isopropoxypyridin-2-amine (Preparation 200), |
| | Black oil, 1.0 g, 41.9% yield. LCMS m/z = 340.8 [M + H]⁺ |
| 218 | 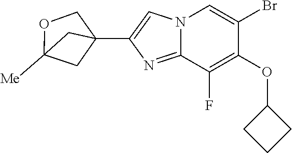 [SMILES: CC12CC(c3cn4cc(Br)c(OC5CCC5)c(F)c4n3)(CO1)C2] |
| | 6-bromo-7-cyclobutoxy-8-fluoro-2-(1-methyl-2-oxabicyclo[2.1.1]hexan-4- |
| | yl)imidazo[1,2-a]pyridine SM: 2-bromo-1-(1-methyl-2- |
| | oxabicyclo[2.1.1]hexan-4-yl)ethan-1-one (Preparation 36) and 5-bromo-4- |
| | cyclobutoxy-3-fluoropyridin-2-amine (Preparation 195), |
| | yellow solid, 340 mg, 63.7% yield. LCMS m/z = 383.0 [M + H]⁺ |
| 219 | 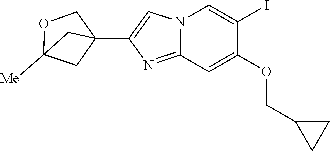 [SMILES: CC12CC(c3cn4cc(I)c(OCC5CC5)cc4n3)(CO1)C2] |
| | 7-(cyclopropylmethoxy)-6-iodo-2-(1-methyl-2-oxabicyclo[2.1.1]hexan-4- |
| | yl)imidazo[1,2-a]pyrimidine SM: 4-(cyclopropylmethoxy)-5-iodopyrimidin- |
| | 2-amine (Preparation 198) and 2-bromo-1-(1-methyl-2- |
| | oxabicyclo[2.1.1]hexan-4-yl)ethan-1-one (Preparation 36), |
| | yellow solid, 670 mg, 32.2% yield. LCMS m/z = 412.0 [M + H]⁺ |

### Preparation 220: 6-Bromo-8-isopropoxy-2-(tetrahydro-2H-pyran-4-yl)imidazo[1,2-a]pyridine

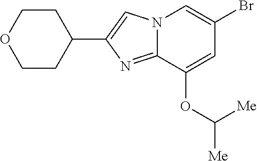 [SMILES: CC(C)Oc1cc(Br)cn2cc(C3CCOCC3)nc12]

was obtained as a yellow solid, 580 mg, 32.9% yield, from 2-bromo-1-(tetrahydro-2H-pyran-4-yl)ethan-1-one and 5-bromo-3-isopropoxypyridin-2-amine (Preparation 200) following the procedure described in Preparations 39 to 42. ¹H NMR (500 MHz, CDCl₃) δ: 1.49 (d, 6H), 1.75-1.79 (m, 2H), 2.04-2.08 (m, 2H), 3.07-3.11 (m, 1H), 3.51-3.57 (m, 2H), 4.04-4.08 (m, 2H), 4.75-4.80 (m, 1H), 6.51 (s, 1H), 7.25 (s, 1H), 7.83 (d, 1H)

### Preparation 221: 2-(3-Oxabicyclo[3.1.0]hexan-6-yl)-6-iodo-7-isopropoxyimidazo[1,2-a]pyridine

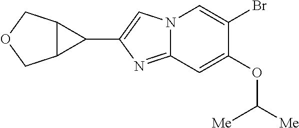 [SMILES: CC(C)Oc1cc2nc(C3C4COCC43)cn2cc1Br]

was obtained as a yellow oil, from 1-(3-oxabicyclo[3.1.0]hexan-6-yl)-2-bromoethan-1-one (Preparation 35) and 5-bromo-4-isopropoxypyridin-2-amine (Preparation 1) following the procedure described in Preparations 39 to 42, 130 mg, 59.4% yield, as a brown oil. LCMS m/z=337.0 [M+H]⁺

### Preparation 222: 2-(3-Oxabicyclo[3.1.0]hexan-6-yl)-6-iodo-7-isopropoxyimidazo[1,2-a]pyrimidine

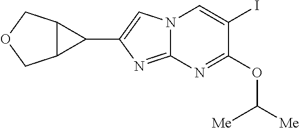 [SMILES: CC(C)Oc1nc2nc(C3C4COCC43)cn2cc1I]

was obtained as a yellow oil, from 1-(3-oxabicyclo[3.1.0]hexan-6-yl)-2-bromoethan-1-one (Preparation 35) and 5-iodo-4-isopropoxypyrimidin-2-amine (Preparation 196), following the procedure described in Preparations 39 to 42, 190 mg, 19.2% yield, as a yellow oil. LCMS m/z=385.9 [M+H]⁺

### Preparation 223: 6-Bromo-7-cyclopropoxy-2-(1-methyl-2-oxabicyclo[2.1.1]hexan-4-yl)imidazo[1,2-a]pyrimidine

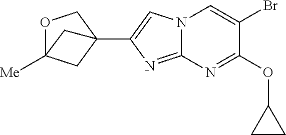 [SMILES: CC12CC(c3cn4cc(Br)c(OC5CC5)nc4n3)(CO1)C2]

was obtained from 5-bromo-4-cyclopropoxypyrimidin-2-amine (Preparation 201) and 2-bromo-1-(1-methyl-2-oxabicyclo[2.1.1]hexan-4-yl)ethan-1-one (Preparation 36), following a similar procedure to that described in Preparations 39 to 42, 74 mg, 12.1% yield. LCMS m/z=351.9 [M+H]⁺

### Preparation 224: 6-Bromo-7-cyclobutoxy-2-(1-methyl-2-oxabicyclo[2.2.1]heptan-4-yl)imidazo[1,2-a]pyridine

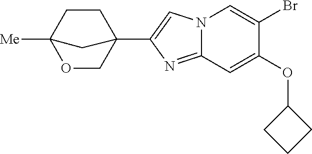 [SMILES: CC12CCC(c3cn4cc(Br)c(OC5CCC5)cc4n3)(CO1)C2]

A mixture of 2-bromo-1-(1-methyl-2-oxabicyclo[2.2.1]heptan-4-yl)ethan-1-one (Preparation 171, 575 mg, 2.47 mmol), 5-bromo-4-cyclobutoxypyridin-2-amine (Preparation 204, Part A, 500 mg, 2.06 mmol) and NaHCO₃ (518 mg, 6.17 mmol) in MeCN (6 mL) and toluene (4 mL) was heated at 90° C. overnight. The reaction mixture was partitioned between EtOAc and brine and the aqueous layer was extracted with EtOAc (×2). The combined organics were dried (Na₂SO₄) and evaporated to dryness in vacuo. The residue was purified by column chromatography on silica gel eluting with EtOAc to afford 6-bromo-7-cyclobutoxy-2-(1-methyl-2-oxabicyclo[2.2.1]heptan-4-yl)imidazo[1,2-a]pyridine as a pale brown oil (636 mg, 81%). ¹H NMR (400 MHz, MeOH-d₄) δ: 1.47 (s, 3H), 1.70-2.00 (m, 6H), 2.10-2.30 (m, 4H), 2.50-2.70 (m, 2H), 3.91 (d, 1H), 4.03 (dd, 1H), 4.84 (d, 1H), 6.71 (s, 1H), 7.50 (s, 1H), 8.60 (s, 1H)

### Preparation 225: 6-Bromo-7-cyclobutoxy-2-(1-methyl-2-oxabicyclo[2.2.2]octan-4-yl)imidazo[1,2-a]pyridine

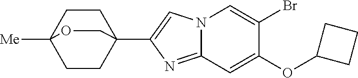 [SMILES: CC12CCC(c3cn4cc(Br)c(OC5CCC5)cc4n3)(CC1)CO2]

A mixture of 2-bromo-1-(1-methyl-2-oxabicyclo[2.2.2]octan-4-yl)ethan-1-one (Preparation 167, 593 mg, 2.40 mmol), 5-bromo-4-cyclobutoxypyridin-2-amine (Preparation 204, Part A, 486 mg, 2.0 mmol) and NaHCO₃ (504 mg, 6.0 mmol) in MeCN (6 mL) and toluene (4 mL) was heated at 90° C. overnight. The reaction was partitioned between EtOAc and brine and the aqueous layer extracted with EtOAc (×2). The combined organics were dried and evaporated to dryness and the residue purified by column chromatography on silica gel eluting with EtOAc to afford 6-bromo-7-cyclobutoxy-2-(1-methyl-2-oxabicyclo[2.2.2]octan-4-yl)imidazo[1,2-a]pyridine as a brown oil (688 mg, 88%). ¹H NMR (400 MHz, MeOH-d₄) δ: 1.15 (s, 3H), 1.73-2.02 (m, 8H), 2.09-2.32 (m, 4H), 2.51-2.66 (m, 2H), 4.04 (s, 2H), 4.79-4.85 (m, 1H), 6.69 (s, 1H), 7.41 (s, 1H), 8.59 (s, 1H)

### Preparation 226: 6-Bromo-7-(cyclopentyloxy)-2-(1-methyl-2-oxabicyclo[2.1.1]hexan-4-yl)imidazo[1,2-a]pyridine

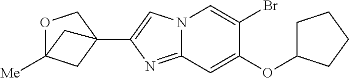 [SMILES: CC12CC(c3cn4cc(Br)c(OC5CCCC5)cc4n3)(CO1)C2]

2-Bromo-1-(1-methyl-2-oxabicyclo[2.1.1]hexan-4-yl)ethan-1-one (Preparation 36, 460 mg, 2.10 mmol) and NaHCO₃ (441 mg, 5.25 mmol) was added to a suspension of 5-bromo-4-(cyclopentoxy)pyridin-2-amine (450 mg, 1.75 mmol) in MeCN (2.10 mL) and toluene (1.40 mL) and the reaction heated at 90° C. in a sealed tube for 1 h. The reaction mixture was evaporated to dryness and the residue purified by column chromatography on silica gel (0-100% EtOAc/heptanes) to afford 6-bromo-7-(cyclopentyloxy)-2-(1-methyl-2-oxabicyclo[2.1.1]hexan-4-yl)imidazo[1,2-a]pyridine, 660 mg, 42.7%. LCMS m/z=377.1 [M+H]⁺

### Preparation 227 to 238

The compounds in the following table were prepared from the appropriate amine and appropriate bromo ketone, following a similar procedure to that described in Preparation 226.

| Prep. no | Structure/Name/Starting materials (SM)/Yield/Data |
|---|---|
| 227 | 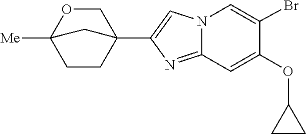 [SMILES: CC12CCC(c3cn4cc(Br)c(OC5CC5)cc4n3)(CO1)C2] |
| | 6-bromo-7-cyclopropoxy-2-(1-methyl-2-oxabicyclo[2.2.1]heptan-4- |
| | yl)imidazo[1,2-a]pyridine SM: 5-bromo-4-cyclopropoxypyridin-2-amine |
| | (Preparation 185) and 2-bromo-1-(1-methyl-2-oxabicyclo[2.2.1]heptan-4- |
| | yl)ethan-1-one (Preparation 171) LCMS m/z = 365.0 [M + H]⁺ |
| 228 | 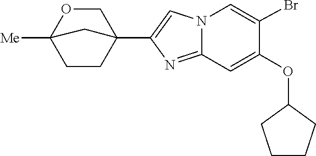 [SMILES: CC12CCC(c3cn4cc(Br)c(OC5CCCC5)cc4n3)(CO1)C2] |
| | 6-bromo-7-(cyclopentyloxy)-2-(1-methyl-2-oxabicyclo[2.2.1]heptan-4- |
| | yl)imidazo[1,2-a]pyridine SM: 5-bromo-4-(cyclopentoxy)pyridin-2-amine |
| | and 2-bromo-1-(1-methyl-2-oxabicyclo[2.2.1]heptan-4-yl)ethan-1-one |
| | (Preparation 171) LCMS m/z = 393 [M + H]⁺ |
| 229 | 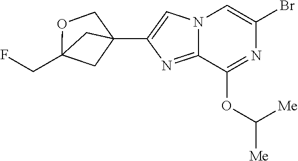 [SMILES: CC(C)Oc1nc(Br)cn2cc(C34COC(CF)(C3)C4)nc12] |
| | 6-bromo-2-(1-(fluoromethyl)-2-oxabicyclo[2.1.1]hexan-4-yl)-8- |
| | isopropoxyimidazo[1,2-a]pyrazine SM: 5-bromo-3-isopropoxypyrazin-2- |
| | amine (Preparation 191) and 2-bromo-1-(1-(fluoromethyl)-2- |
| | oxabicyclo[2.1.1]hexan-4-yl)ethan-1-one (Preparation 166) |
| | 400 mg, 62.8% yield. LCMS m/z = 372.1 [M + H]⁺ |

Using 2-bromo-1-(1-methyl-2-oxabicyclo[2.1.1]hexan-4-yl)ethan-1-one as the appropriate
bromo ketone (Preparation 36)

| | |
|---|---|
| 230 | 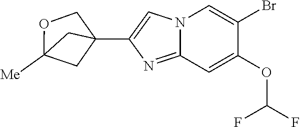 [SMILES: CC12CC(c3cn4cc(Br)c(OC(F)F)cc4n3)(CO1)C2] |
| | 6-bromo-7-(difluoromethoxy)-2-(1-methyl-2-oxabicyclo[2.1.1]hexan-4- |
| | yl)imidazo[1,2-a]pyridine SM: 5-bromo-4-(difluoromethoxy)pyridin-2-amine |
| | (Preparation 184) 400 mg, 53.3% yield. LCMS m/z = 361.0 [M + H]⁺ |
| 231 | 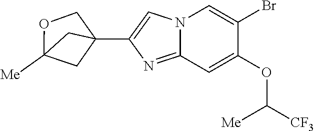 [SMILES: CC(Oc1cc2nc(C34COC(C)(C3)C4)cn2cc1Br)C(F)(F)F] |
| | 6-bromo-2-(1-methyl-2-oxabicyclo[2.1.1]hexan-4-yl)-7-((1,1,1- |
| | trifluoropropan-2-yl)oxy)imidazo[1,2-a]pyridine |
| | SM: 5-bromo-4-((1,1,1-trifluoropropan-2-yl)oxy)pyridin-2-amine |
| | (Preparation 186) 500 mg, 70.5% yield. LCMS m/z = 405.1 [M + H]⁺ |
| 232 | 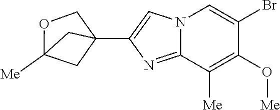 [SMILES: COc1c(Br)cn2cc(C34COC(C)(C3)C4)nc2c1C] |
| | 6-bromo-7-methoxy-8-methyl-2-(1-methyl-2-oxabicyclo[2.1.1]hexan-4- |
| | yl)imidazo[1,2-a]pyridine SM: 5-bromo-4-methoxy-3-methylpyridin-2-amine |
| | (Preparation 187) 330 mg, 70.9% yield LCMS m/z = 339.1 [M + H]⁺ |
| 233 | |
| | 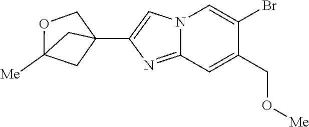 [SMILES: COCc1cc2nc(C34COC(C)(C3)C4)cn2cc1Br] |
| | 6-bromo-7-(methoxymethyl)-2-(1-methyl-2-oxabicyclo[2.1.1]hexan-4- |
| | yl)imidazo[1,2-a]pyridine SM: 5-bromo-4-(methoxymethyl)pyridin-2-amine |
| | (Preparation 188) 520 mg, 60.9% yield. LCMS m/z = 337.0 [M + H]⁺ |
| 234 | 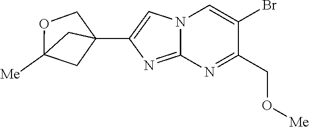 [SMILES: COCc1nc2nc(C34COC(C)(C3)C4)cn2cc1Br] |
| | 6-bromo-7-(methoxymethyl)-2-(1-methyl-2-oxabicyclo[2.1.1]hexan-4- |
| | yl)imidazo[1,2-a]pyrimidine SM: 5-bromo-4-(methoxymethyl)pyrimidin-2- |
| | amine (Preparation 193) 130 mg, 12.0% yield. LCMS m/z = 338.0 [M + H]⁺ |
| 235 | 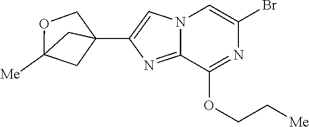 [SMILES: CCCOc1nc(Br)cn2cc(C34COC(C)(C3)C4)nc12] |
| | 6-bromo-2-(1-methyl-2-oxabicyclo[2.1.1]hexan-4-yl)-8-propoxyimidazo[1,2- |
| | a]pyrazine SM: 5-bromo-3-propoxypyrazin-2-amine (Preparation 189) |
| | 300 mg, 66.0% yield. LCMS m/z = 354.1 [M + H]⁺ |
| 236 | 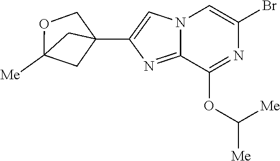 [SMILES: CC(C)Oc1nc(Br)cn2cc(C34COC(C)(C3)C4)nc12] |
| | 6-bromo-8-isopropoxy-2-(1-methyl-2-oxabicyclo[2.1.1]hexan-4- |
| | yl)imidazo[1,2-a]pyrazine SM: 5-bromo-3-isopropoxypyrazin-2-amine |
| | (Preparation 191) 350 mg, 57.8% yield. LCMS m/z = 354.1 [M + H]⁺ |
| 237 | 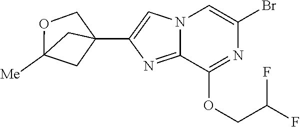 [SMILES: CC12CC(c3cn4cc(Br)nc(OCC(F)F)c4n3)(CO1)C2] |
| | 6-bromo-8-(2,2-difluoroethoxy)-2-(1-methyl-2-oxabicyclo[2.1.1]hexan-4- |
| | yl)imidazo[1,2-a]pyrazine SM: 5-bromo-3-(2,2-difluoroethoxy)pyrazin-2- |
| | amine (Preparation 190) 220 mg, 42.6% yield. LCMS m/z = 376.1 [M + H]⁺ |
| 238 | 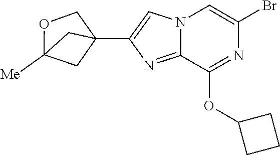 [SMILES: CC12CC(c3cn4cc(Br)nc(OC5CCC5)c4n3)(CO1)C2] |
| | 6-bromo-8-cyclobutoxy-2-(1-methyl-2-oxabicyclo[2.1.1]hexan-4- |
| | yl)imidazo[1,2-a]pyrazine SM: 5-bromo-3-cyclobutoxypyrazin-2-amine |
| | (Preparation 192) 340 mg, 65.3% yield. LCMS m/z = 364.1 [M + H]⁺ |

### Preparation 239: 7-(Benzyloxy)-6-bromo-2-(tert-butyl)imidazo[1,2-a]pyridine

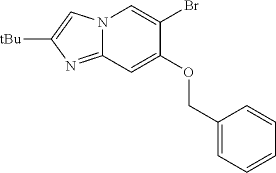 [SMILES: CC(C)(C)c1cn2cc(Br)c(OCc3ccccc3)cc2n1]

K₂CO₃ (7.92 g, 57.32 mmol) was added to a solution of 4-(benzyloxy)-5-bromopyridin-2-amine (Preparation 199, 8.0 g, 28.66 mmol) and 1-bromo-3,3-dimethyl-butan-2-one (6.41 g, 35.83 mmol) in MeCN (50 mL) and the reaction stirred at 80° C. overnight. The cooled reaction mixture was filtered, the filtrate concentrated in vacuo and the residue purified by column chromatography on silica gel eluting with 0-100% EtOAc-heptanes to afford 7-(benzyloxy)-6-bromo-2-(tert-butyl)imidazo[1,2-a]pyridine. LCMS m/z=360.0 [M+H]⁺

### Preparation 240: 7-(Benzyloxy)-6-bromo-2-(1-methyl-2-oxabicyclo[2.1.1]hexan-4-yl)imidazo[1,2-a]pyridine

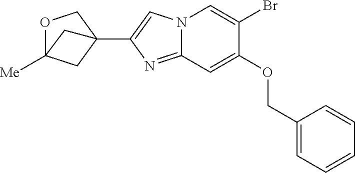 [SMILES: CC12CC(c3cn4cc(Br)c(OCc5ccccc5)cc4n3)(CO1)C2]

was obtained from 4-(benzyloxy)-5-bromopyridin-2-amine (Preparation 199) and 2-bromo-1-(1-methyl-2-oxabicyclo[2.1.1]hexan-4-yl)ethan-1-one (Preparation 36) following the procedure described in Preparation 239. LCMS m/z=401.0 [M+H]⁺

### Preparation 241: 6-Bromo-8-fluoro-2-(1-(fluoromethyl)-2-oxabicyclo[2.1.1]hexan-4-yl)-7-isopropoxyimidazo[1,2-a]pyridine

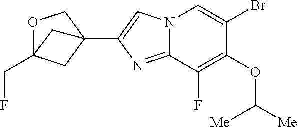 [SMILES: CC(C)Oc1c(Br)cn2cc(C34COC(CF)(C3)C4)nc2c1F]

was obtained from 5-bromo-3-fluoro-4-isopropoxypyridin-2-amine (Preparation 194) and 2-bromo-1-(1-(fluoromethyl)-2-oxabicyclo[2.1.1]hexan-4-yl)ethan-1-one (Preparation 166), following a similar procedure to that described in Preparation 239. LCMS m/z=388.9 [M+H]⁺

### Preparation 242: Methyl 8-bromo-2-cyclopropylimidazo[1,2-a]pyrazine-6-carboxylate

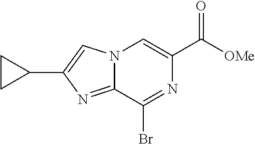 [SMILES: COC(=O)c1cn2cc(C3CC3)nc2c(Br)n1]

A mixture of methyl 5-amino-6-bromopyrazine-2-carboxylate (1.0 g, 4.31 mmol), NaHCO₃ (1.09 g, 12.93 mmol) and 2-bromo-1-cyclopropylethan-1-one (878.1 mg, 5.39 mmol) in MeCN:toluene (10 mL) was stirred at 90° C. for 17 h. The cooled reaction mixture was filtered through a pad of Celite® and the filtrate concentrated in vacuo. The residue was purified by Isco purification system (0-30% 3:1 EtOAc:EtOH in heptanes) to afford methyl 8-bromo-2-cyclopropylimidazo[1,2-a]pyrazine-6-carboxylate (437 mg, 34.2% yield) as a brown solid. LCMS m/z=295.9 [M+H]⁺

### Preparation 243: Methyl 7-cyclobutoxy-2-(1-methyl-2-oxabicyclo[2.1.1]hexan-4-yl)imidazo[1,2-a]pyrimidine-6-carboxylate

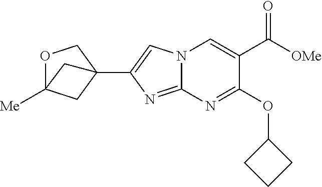 [SMILES: COC(=O)c1cn2cc(C34COC(C)(C3)C4)nc2nc1OC1CCC1]

To a solution of 7-cyclobutoxy-6-iodo-2-(1-methyl-2-oxabicyclo[2.1.1]hexan-4-yl)imidazo[1,2-a]pyrimidine (Preparation 214, 431 mg, 1.05 mmol) in MeOH (20 mL) was added Pd(dppf)Cl₂ (76.8 mg, 0.105 mmol) and TEA (1.06 g, 10.50 mmol) and the mixture was degassed with CO, then stirred at 80° C. under CO (50 psi) for 16 h. The cooled mixture was concentrated in vacuo and the residue purified by CombiFlash® (PE/EtOAc=0 to 1/1) to afford methyl 7-cyclobutoxy-2-(1-methyl-2-oxabicyclo[2.1.1]hexan-4-yl)imidazo[1,2-a]pyrimidine-6-carboxylate (283 mg, 76.1% yield) as a brown solid. LCMS m/z=344.2 [M+H]⁺

### Preparations 244 to 252

To a solution of the appropriate halide (1.0 equiv.) in MeOH was added TEA (10.0 equiv.) and Pd(dppf)Cl₂ (0.1 equiv. to 0.2 equiv.) at 15° C. under N₂. The mixture was stirred at 80° C. under CO at 50 psi for 24 h. The cooled reaction was filtered through Celite® and the filtrate concentrated in vacuo. The residue was purified by column chromatography on silica gel using Combiflash® eluting with DCM/EtOAc or PE/EtOAc, at an appropriate gradient, to afford the title compound.

| Prep. No | Structure/Name/Starting Material (SM)/Yield/Data |
|---|---|
| 244 | 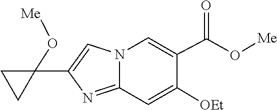 [SMILES: CCOc1cc2nc(C3(OC)CC3)cn2cc1C(=O)OC] |
| | methyl 7-ethoxy-2-(1-methoxycyclopropyl)imidazo[1,2-a]pyridine-6- |
| | carboxylate SM: 6-bromo-7-ethoxy-2-(1-methoxycyclopropyl)imidazo[1,2- |
| | a]pyridine (Preparation 215) (500 mg, 89.2% yield) as a red oil. LCMS m/z = |
| | 290.9 [M + H]⁺ |
| 245 | 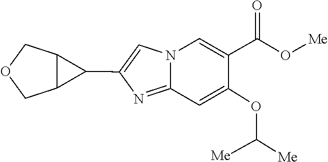 [SMILES: COC(=O)c1cn2cc(C3C4COCC43)nc2cc1OC(C)C] |
| | methyl 2-(3-oxabicyclo[3.1.0]hexan-6-yl)-7-isopropoxyimidazo[1,2- |
| | a]pyridine-6-carboxylate SM: 2-(3-oxabicyclo[3.1.0]hexan-6-yl)-6-iodo-7- |
| | isopropoxyimidazo[1,2-a]pyridine (Preparation 221) 60 mg, 53.3% yield. |
| | LCMS m/z = 317.1 [M + H]⁺ |
| 246 | 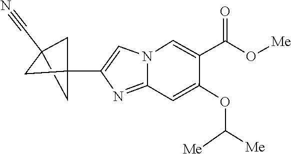 [SMILES: COC(=O)c1cn2cc(C34CC(C#N)(C3)C4)nc2cc1OC(C)C] |
| | methyl 2-(3-cyanobicyclo[1.1.1]pentan-1-yl)-7-isopropoxyimidazo[1,2- |
| | a]pyridine-6-carboxylate SM: 3-(6-bromo-7-isopropoxyimidazo[1,2-a]pyridin- |
| | 2-yl)bicyclo[1.1.1]pentane-1-carbonitrile (Preparation 216) |
| | 130 mg, 65.7% yield, as a yellow solid. LCMS m/z = 326.0 [M + H]⁺ |
| 247 | 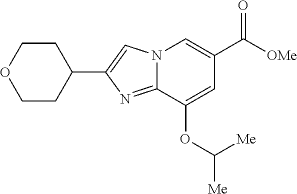 [SMILES: COC(=O)c1cc(OC(C)C)c2nc(C3CCOCC3)cn2c1] |
| | Methyl 8-isopropoxy-2-(tetrahydro-2H-pyran-4-yl)imidazo[1,2-a]pyridine-6- |
| | carboxylate SM: 6-bromo-8-isopropoxy-2-(tetrahydro-2H-pyran-4- |
| | yl)imidazo[1,2-a]pyridine (Preparation 220) |
| | 430 mg, 76.3% yield as a yellow solid. LCMS m/z = 319.0 [M + H]⁺ |
| 248 | 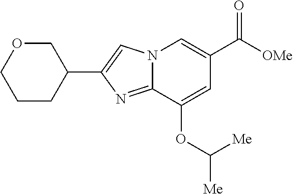 [SMILES: COC(=O)c1cc(OC(C)C)c2nc(C3CCCOC3)cn2c1] |
| | Methyl 8-isopropoxy-2-(tetrahydro-2H-pyran-3-yl)imidazo[1,2-a]pyridine-6- |
| | carboxylate SM: 6-bromo-8-isopropoxy-2-(tetrahydro-2H-pyran-3- |
| | yl)imidazo[1,2-a]pyridine (Preparation 217) 1.0 g, crude, as black oil. LCMS |
| | m/z = 319.2 [M + H]⁺ |
| 249 | 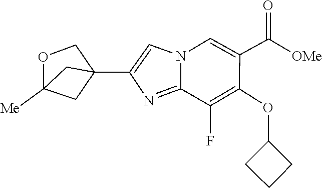 [SMILES: COC(=O)c1cn2cc(C34COC(C)(C3)C4)nc2c(F)c1OC1CCC1] |
| | methyl 7-cyclobutoxy-8-fluoro-2-(1-methyl-2-oxabicyclo[2.1.1]hexan-4- |
| | yl)imidazo[1,2-a]pyridine-6-carboxylate SM: 6-bromo-7-cyclobutoxy-8-fluoro- |
| | 2-(1-methyl-2-oxabicyclo[2.1.1]hexan-4-yl)imidazo[1,2-a]pyridine |
| | (Preparation 218) 270 mg, 78.3% yield. LCMS m/z = 361.0 [M + H]⁺ |
| 250 | 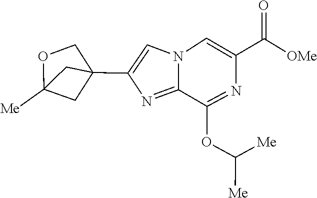 [SMILES: COC(=O)c1cn2cc(C34COC(C)(C3)C4)nc2c(OC(C)C)n1] |
| | Methyl 8-isopropoxy-2-(1-methyl-2-oxabicyclo[2.1.1]hexan-4-yl)imidazo[1,2- |
| | a]pyrazine-6-carboxylate SM: 6-bromo-8-isopropoxy-2-(1-methyl-2- |
| | oxabicyclo[2.1.1]hexan-4-yl)imidazo[1,2-a]pyrazine (Preparation 236) |
| | 280 mg, 89.3% yield as a white solid. ¹H NMR (400 MHz, CDCl₃) δ: 1.52 (d, |
| | 6H), 1.53 (s, 3H), 1.97-1.99 (m, 2H), 2.10-2.12 (m, 2H), 3.96 (s, 3H), 4.09 (s, |
| | 2H), 5.72-5.79 (m, 1H), 7.50 (s, 1H), 8.52 (s, 1H) |
| 251 | 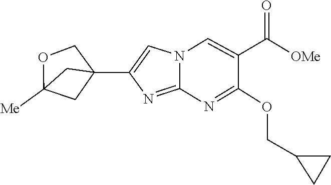 [SMILES: COC(=O)c1cn2cc(C34COC(C)(C3)C4)nc2nc1OCC1CC1] |
| | methyl 7-(cyclopropylmethoxy)-2-(1-methyl-2-oxabicyclo[2.1.1]hexan-4- |
| | yl)imidazo[1,2-a]pyrimidine-6-carboxylate SM: 7-(cyclopropylmethoxy)-6- |
| | iodo-2-(1-methyl-2-oxabicyclo[2.1.1]hexan-4-yl)imidazo[1,2-a]pyrimidine |
| | (Preparation 219) 532.0 mg, 96.6% yield LCMS m/z = 343.9 [M + H]⁺ |
| 252 | 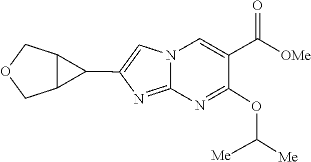 [SMILES: COC(=O)c1cn2cc(C3C4COCC43)nc2nc1OC(C)C] |
| | methyl 2-(3-oxabicyclo[3.1.0]hexan-6-yl)-7-isopropoxyimidazo[1,2- |
| | a]pyrimidine-6-carboxylate SM: 2-(3-oxabicyclo[3.1.0]hexan-6-yl)-6-iodo-7- |
| | isopropoxyimidazo[1,2-a]pyrimidine (Preparation 222) |
| | 130 mg, 77.2% yield as a yellow solid. LCMS m/z = 318.1 [M + H]⁺ |

### Preparation 253: Methyl 8-fluoro-7-isopropoxy-2-(tetrahydro-2H-pyran-4-yl)imidazo[1,2-a]pyridine-6-carboxylate

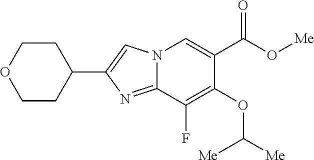 [SMILES: COC(=O)c1cn2cc(C3CCOCC3)nc2c(F)c1OC(C)C]

A mixture of phenyl 6-amino-5-fluoro-4-isopropoxynicotinate (Preparation 203, 120 mg, 0.413 mmol), 2-bromo-1-tetrahydropyran-4-yl-ethanone (85.6 mg, 0.413 mmol) and NaHCO₃ (104.2 mg, 1.24 mmol) in EtOH (1.03 mL) was heated at 80° C. overnight. The cooled mixture was adsorbed onto silica gel and purified by column chromatography to provide phenyl 8-fluoro-7-isopropoxy-2-(tetrahydro-2H-pyran-4-yl)imidazo[1,2-a]pyridine-6-carboxylate (90 mg, 54.6% yield). This product was dissolved in MeOH, NaHCO₃ (189.7 mg) added and the solution heated at 45° C. overnight. The cooled solution was filtered, the filtrate concentrated in vacuo, and the residue purified by column chromatography on silica gel to afford methyl 8-fluoro-7-isopropoxy-2-(tetrahydro-2H-pyran-4-yl)imidazo[1,2-a]pyridine-6-carboxylate (60 mg, 79.0% yield). LCMS m/z=337.0 [M+H]⁺

### Preparation 254: Methyl 2-cyclopropyl-7-isopropoxyimidazo[1,2-a]pyridine-6-carboxylate

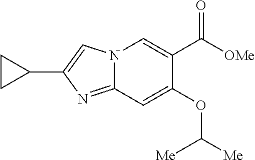 [SMILES: COC(=O)c1cn2cc(C3CC3)nc2cc1OC(C)C]

A mixture of methyl 6-amino-4-isopropoxynicotinate (Preparation 2, 20 g, 95.1 mmol), 2-bromo-1-cyclopropyl-ethan-1-one (18.61 g, 114.2 mmol) and NaHCO₃ (7.99 g, 95.1 mmol) in EtOH (200 mL) was heated at 80° C. for 96 h in a sealed vessel. The mixture was cooled to rt, diluted with H₂O (100 mL) and extracted with DCM (3×100 mL). The combined organics were washed with brine, dried (Na₂SO₄) and evaporated to dryness in vacuo. The residue was crystallized from hexane/MeCN (200 mL/50 mL), the solid collected and washed with hexane and air-dried to give methyl 2-cyclopropyl-7-isopropoxyimidazo[1,2-a]pyridine-6-carboxylate as a yellow solid (14 g, 41.5%). LCMS m/z=275.2 [M+H]⁺

### Preparation 255: Methyl 7-(benzyloxy)-2-cyclopropylimidazo[1,2-a]pyridine-6-carboxylate

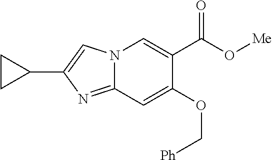 [SMILES: COC(=O)c1cn2cc(C3CC3)nc2cc1OCc1ccccc1]

was obtained as a yellow solid from methyl 6-amino-4-(benzyloxy)nicotinate (Preparation 205) and 2-chloro-1-cyclopropyl-ethan-1-one, following a similar procedure to that described in Preparation 254, 8.5 g, 30% yield. LCMS m/z=323.2 [M+H]⁺.

### Preparation 256: Methyl 7-isopropoxy-2-(1-methyl-2-oxabicyclo[2.2.1]heptan-4-yl)imidazo[1,2-a]pyridine-6-carboxylate

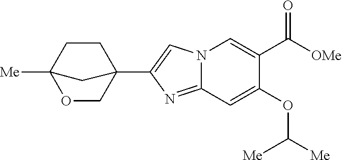 [SMILES: COC(=O)c1cn2cc(C34CCC(C)(C3)OC4)nc2cc1OC(C)C]

A mixture of 2-bromo-1-(1-methyl-2-oxabicyclo[2.2.1]heptan-4-yl)ethan-1-one (Preparation 171, 489.5 mg, 2.10 mmol), methyl 6-amino-4-isopropoxynicotinate (Preparation 2, 420.5 mg, 2.0 mmol) and NaHCO₃ (504 mg, 6.0 mmol) in MeCN (6 mL) and toluene (4 mL) was heated at 90° C. overnight. The cooled reaction mixture was partitioned between EtOAc and brine and the aqueous layer extracted with EtOAc (2×). The combined organics were dried (Na₂SO₄) and evaporated to dryness in vacuo. The residue was purified by silica gel column chromatography (EtOAc) to afford methyl 7-isopropoxy-2-(1-methyl-2-oxabicyclo[2.2.1]heptan-4-yl)imidazo[1,2-a]pyridine-6-carboxylate as a pale-yellow oil (542 mg, 78%). LCMS m/z=345.2 [M+H]⁺

### Preparation 257: Methyl 7-isopropoxy-2-(1-methyl-2-oxabicyclo[2.2.2]octan-4-yl)imidazo[1,2-a]pyridine-6-carboxylate

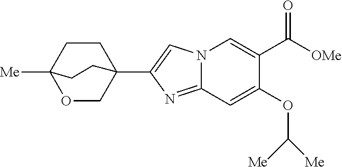 [SMILES: COC(=O)c1cn2cc(C34CCC(C)(CC3)OC4)nc2cc1OC(C)C]

A mixture of NaHCO₃ (718 mg, 8.55 mmol), methyl 6-amino-4-isopropoxynicotinate (Preparation 2, 650 mg, 2.85 mmol) and 2-bromo-1-(1-methyl-2-oxabicyclo[2.2.2]octan-4-yl)ethan-1-one (Preparation 167, 704 mg, 2.85 mmol) in MeCN (4 mL) and toluene (4 mL) was stirred at 90° C. overnight. MeOH and SiO₂ were added and the mixture was evaporated to dryness. The residue was purified by column chromatography on silica gel (0-50% 3/1 EtOAc/EtOH in heptanes) to afford methyl 7-isopropoxy-2-(1-methyl-2-oxabicyclo[2.2.2]octan-4-yl)imidazo[1,2-a]pyridine-6-carboxylate, 900 mg, 88% yield. LCMS m/z=359.2 [M+H]⁺.

### Preparation 258: Ethyl 7-(cyclopentyloxy)-2-(1-methyl-2-oxabicyclo[2.2.1]heptan-4-yl)imidazo[1,2-a]pyrimidine-6-carboxylate

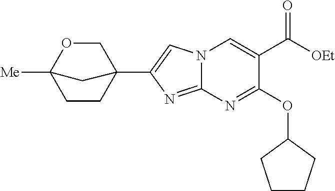 [SMILES: CCOC(=O)c1cn2cc(C34CCC(C)(C3)OC4)nc2nc1OC1CCCC1]

was obtained, 300 mg, 33.4% yield, from 2-bromo-1-(1-methyl-2-oxabicyclo[2.2.1]heptan-4-yl)ethan-1-one (Preparation 171) and ethyl 2-amino-4-(cyclopentyloxy)pyrimidine-5-carboxylate (Preparation 211) following the procedure described in Preparation 257. LCMS m/z=386.3 [M+H]⁺

### Preparation 259: Methyl 2-cyclopropyl-8-ethoxyimidazo[1,2-a]pyrazine-6-carboxylate

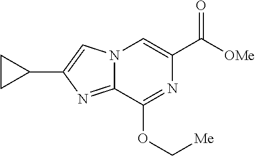 [SMILES: CCOc1nc(C(=O)OC)cn2cc(C3CC3)nc12]

was obtained as a light yellow solid, 381 mg, 57.4% yield, from 2-bromo-1-cyclopropylethan-1-one and methyl 5-amino-6-ethoxypyrazine-2-carboxylate (Preparation 208), following the procedure described in Preparation 257. LCMS m/z=262.1 [M+H]⁺

### Preparation 260: Methyl 8-(benzyloxy)-2-cyclopropylimidazo[1,2-a]pyrazine-6-carboxylate

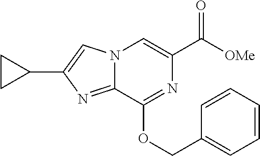 [SMILES: COC(=O)c1cn2cc(C3CC3)nc2c(OCc2ccccc2)n1]

was obtained, 991 mg, 79.4% yield, from 2-bromo-1-cyclopropylethan-1-one and methyl 5-amino-6-(benzyloxy)pyrazine-2-carboxylate (Preparation 210), following the procedure described in Preparation 257. LCMS m/z=324.1 [M+H]⁺

### Preparation 261: Methyl 2-(2-oxabicyclo[2.1.1]hexan-4-yl)-7-isopropoxyimidazo[1,2-a]pyridine-6-carboxylate

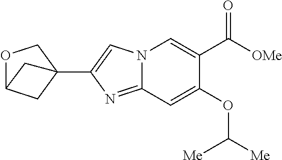 [SMILES: COC(=O)c1cn2cc(C34COC(C3)C4)nc2cc1OC(C)C]

A mixture of methyl 6-amino-4-isopropoxynicotinate (Preparation 2, 1.30 g, 6.18 mmol), 1-(2-oxabicyclo[2.1.1]hexan-4-yl)-2-bromoethan-1-one (Preparation 168, 1.52 g, 7.42 mmol) and NaHCO₃ (623 mg, 7.42 mmol) in MeCN (25 mL) and toluene (25 mL) was heated in a sealed tube at 90° C. for 14 h. The mixture was cooled to rt, diluted with H₂O (100 mL) and extracted with DCM (3×50 mL). The combined organics were washed with brine, dried (Na₂SO₄) and evaporated to dryness in vacuo to afford methyl 2-(2-oxabicyclo[2.1.1]hexan-4-yl)-7-isopropoxyimidazo[1,2-a]pyridine-6-carboxylate as a yellow oil (1.65 g). LCMS m/z=317.2 [M+H]⁺

### Preparation 262: Methyl 7-cyclobutoxy-2-(1-methyl-2-oxabicyclo[2.1.1]hexan-4-yl)imidazo[1,2-a]pyridine-6-carboxylate

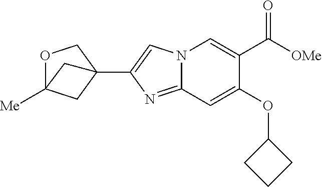 [SMILES: COC(=O)c1cn2cc(C34COC(C)(C3)C4)nc2cc1OC1CCC1]

was obtained as a yellow oil, 4.5 g, crude, from methyl 6-amino-4-cyclobutoxynicotinate (Preparation 204) and 2-bromo-1-(1-methyl-2-oxabicyclo[2.1.1]hexan-4-yl)ethan-1-one (Preparation 36) following the procedure described in Preparation 261, LCMS m/z=343.0 [M+H]⁺.

### Preparation 263: Methyl 7-(benzyloxy)-2-(1-methyl-2-oxabicyclo[2.1.1]hexan-4-yl)imidazo[1,2-a]pyridine-6-carboxylate

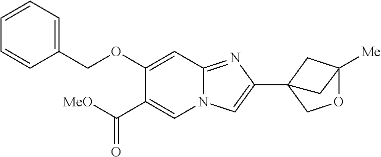 [SMILES: COC(=O)c1cn2cc(C34COC(C)(C3)C4)nc2cc1OCc1ccccc1]

was obtained as a brown solid, 16.2 g, from methyl 6-amino-4-(benzyloxy)nicotinate (Preparation 205) and 2-bromo-1-(1-methyl-2-oxabicyclo[2.1.1]hexan-4-yl)ethan-1-one (Preparation 36), following the procedure described in Preparation 261. LCMS m/z=379.2 [M+H]⁺

### Preparation 264: Methyl 8-ethoxy-2-(1-methyl-2-oxabicyclo[2.2.1]heptan-4-yl)imidazo[1,2-a]pyrazine-6-carboxylate

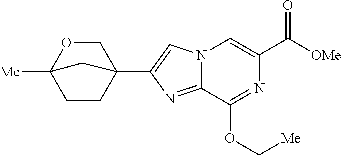 [SMILES: CCOc1nc(C(=O)OC)cn2cc(C34CCC(C)(C3)OC4)nc12]

was obtained as a yellow solid from methyl 5-amino-6-ethoxypyrazine-2-carboxylate (Preparation 208) and 2-bromo-1-(1-methyl-2-oxabicyclo[2.2.1]heptan-4-yl)ethan-1-one (Preparation 171) following a similar procedure to that described in Preparation 107 (1.41 g, 83.9% yield). LCMS m/z=332.2 [M+H]⁺

### Preparation 265: Isopropyl 7-isopropoxy-2-(1,3,3-trimethyl-2-oxabicyclo[2.1.1]hexan-4-yl)imidazo[1,2-a]pyrimidine-6-carboxylate

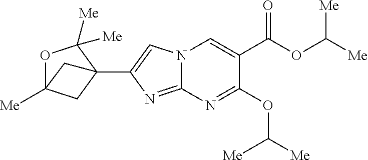 [SMILES: CC(C)OC(=O)c1cn2cc(C34CC(C)(C3)OC4(C)C)nc2nc1OC(C)C]

was obtained, 330 mg, 68.1% yield, from isopropyl 2-amino-4-isopropoxypyrimidine-5-carboxylate (Preparation 206) and 2-bromo-1-(1,3,3-trimethyl-2-oxabicyclo[2.1.1]hexan-4-yl)ethan-1-one (Preparation 169) following the procedure described in Preparation 107. LCMS m/z=388.2 [M+H]⁺

### Preparation 266: Isopropyl 8-chloro-7-isopropoxy-2-(1-methyl-2-oxabicyclo[2.1.1]hexan-4-yl)imidazo[1,2-a]pyridine-6-carboxylate

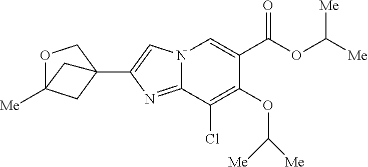 [SMILES: CC(C)OC(=O)c1cn2cc(C34COC(C)(C3)C4)nc2c(Cl)c1OC(C)C]

To a solution of isopropyl 6-amino-5-chloro-4-isopropoxynicotinate (Preparation 183, 240 mg, 0.880 mmol) in t-BuOH (20 mL) was added 2-bromo-1-(1-methyl-2-oxabicyclo[2.1.1]hexan-4-yl)ethan-1-one (Preparation 36, 300 mg, 1.37 mmol) and NaHCO₃ (147.8 mg, 1.76 mmol) and the reaction stirred at 90° C. for 16 h. The cooled mixture was concentrated in vacuo and the residue purified by CombiFlash® (PE/EtOAc=3/1) to afford isopropyl 8-chloro-7-isopropoxy-2-(1-methyl-2-oxabicyclo[2.1.1]hexan-4-yl)imidazo[1,2-a]pyridine-6-carboxylate (310 mg, 80.7% yield) as a brown oil. LCMS m/z=393.0 [M+H]⁺

### Preparation 267: Isopropyl 2-(1-(fluoromethyl)-2-oxabicyclo[2.1.1]hexan-4-yl)-7-isopropoxyimidazo[1,2-a]pyrimidine-6-carboxylate

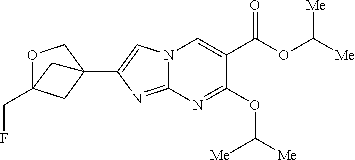 [SMILES: CC(C)OC(=O)c1cn2cc(C34COC(CF)(C3)C4)nc2nc1OC(C)C]

A mixture of NaHCO₃ (315 mg, 3.75 mmol), isopropyl 2-amino-4-isopropoxypyrimidine-5-carboxylate (Preparation 206, 299 mg, 1.25 mmol) and 2-bromo-1-(1-(fluoromethyl)-2-oxabicyclo[2.1.1]hexan-4-yl)ethan-1-one (Preparation 166, 370.4 mg, 1.56 mmol) in MeCN (3 mL) and toluene (3 mL) was stirred at 90° C. overnight. MeOH and SiO₂ were added and the mixture was evaporated to dryness. The residue was purified by dry load silica gel column chromatography (0-40%, 3/1 EtOAc/EtOH in heptanes) to afford isopropyl 241-(fluoromethyl)-2-oxabicyclo[2.1.1]hexan-4-yl)-7-isopropoxyimidazo[1,2-a]pyrimidine-6-carboxylate (170 mg, 36.0%). LCMS m/z=378.2 [M+H]⁺

### Preparation 268: Isopropyl 7-isopropoxy-2-(4-methyl-2-oxabicyclo[2.2.2]octan-1-yl)imidazo[1,2-a]pyrimidine-6-carboxylate

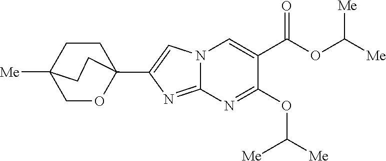 [SMILES: CC(C)OC(=O)c1cn2cc(C34CCC(C)(CC3)CO4)nc2nc1OC(C)C]

A mixture of NaHCO₃ (685 mg, 8.15 mmol), isopropyl 2-amino-4-isopropoxypyrimidine-5-carboxylate (Preparation 206, 650 mg, 2.72 mmol) and 2-bromo-1-(1-methyl-2-oxabicyclo[2.2.2]octan-4-yl)ethan-1-one (Preparation 167, 839 mg, 3.40 mmol) in MeCN (3.8 mL) and toluene (3 mL) was stirred at 90° C. overnight. MeOH and SiO₂ were added and the mixture was evaporated to dryness. The residue was purified by dry load silica gel column chromatography (0-50%, 3/1 EtOAc/EtOH in heptanes) to afford isopropyl 7-isopropoxy-2-(4-methyl-2-oxabicyclo[2.2.2]octan-1-yl)imidazo[1,2-a]pyrimidine-6-carboxylate (700 mg, 66.0%). LCMS m/z=388.5 [M+H]⁺

### Preparation 269: Methyl 3-fluoro-7-isopropoxy-2-(1-methyl-2-oxabicyclo[2.1.1]hexan-4-yl)imidazo[1,2-a]pyridine-6-carboxylate

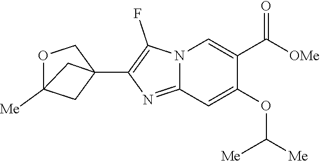 [SMILES: COC(=O)c1cn2c(F)c(C34COC(C)(C3)C4)nc2cc1OC(C)C]

F-TEDA (167.6 mg, 0.473 mmol) was added to a mixture of methyl 7-isopropoxy-2-(1-methyl-2-oxabicyclo[2.1.1]hexan-4-yl)imidazo[1,2-a]pyridine-6-carboxylate (Preparation 60, 499 mg, 1.51 mmol) and N,N-dimethylpyridin-4-amine (369 mg, 3.02 mmol) in CHCl₃ (5.44 mL) and water (604.4 μL) at 0° C. and the reaction stirred at rt overnight. The reaction was quenched with NaHCO₃, extracted with EtOAc and the combined organic extracts dried over MgSO₄, filtered and concentrated in vacuo. The residue was purified by silica gel column chromatography (0-40%, EtOAc/EtOH 3:1 in heptanes) to afford methyl 3-fluoro-7-isopropoxy-2-(1-methyl-2-oxabicyclo[2.1.1]hexan-4-yl)imidazo[1,2-a]pyridine-6-carboxylate, 170 mg, 32.3% yield, LCMS m/z=349.2 [M+H]⁺

### Preparation 270: Phenyl 7-cyclobutoxy-2-(1-methyl-2-oxabicyclo[2.2.1]heptan-4-yl)imidazo[1,2-a]pyridine-6-carboxylate

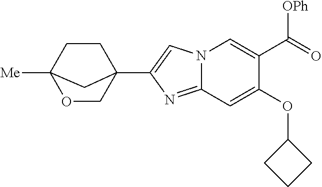 [SMILES: CC12CCC(c3cn4cc(C(=O)Oc5ccccc5)c(OC5CCC5)cc4n3)(CO1)C2]

TEA (344 mg, 3.40 mmol) was added to a mixture of 6-bromo-7-cyclobutoxy-2-(1-methyl-2-oxabicyclo[2.2.1]heptan-4-yl)imidazo[1,2-a]pyridine (Preparation 224, 512 mg, 1.36 mmol), Pd(OAc)₂ (21.4 mg, 0.095 mmol), Xantphos (63.0 mg, 0.109 mmol) and phenyl formate (415 mg, 3.40 mmol) in MeCN (6 mL) and the mixture heated at 80° C. for 4.5 h. The cooled reaction was partitioned between EtOAc and brine, the aqueous layer extracted with EtOAc and the combined organics were evaporated to dryness in vacuo. The residue was purified by column chromatography on silica gel eluting with EtOAc to afford phenyl 7-cyclobutoxy-2-(1-methyl-2-oxabicyclo[2.2.1]heptan-4-yl)imidazo[1,2-a]pyridine-6-carboxylate as a pale-yellow oil (499 mg, 87.0%). LCMS m/z=419.3 [M+H]⁺

### Preparation 271: Phenyl 7-cyclobutoxy-2-(1-methyl-2-oxabicyclo[2.2.2]octan-4-yl)imidazo[1,2-a]pyridine-6-carboxylate

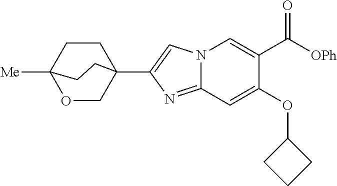 [SMILES: CC12CCC(c3cn4cc(C(=O)Oc5ccccc5)c(OC5CCC5)cc4n3)(CC1)CO2]

TEA (445.2 mg, 4.40 mmol) was added to a mixture of 6-bromo-7-cyclobutoxy-2-(1-methyl-2-oxabicyclo[2.2.2]octan-4-yl)imidazo[1,2-a]pyridine (Preparation 225, 688 mg, 1.76 mmol), Pd(OAc)₂ (19.7 mg, 0.088 mmol), Xantphos (81.5 mg, 0.142 mmol) and phenyl formate (496 mg, 4.07 mmol) in MeCN (8 mL) at rt and the reaction heated at 80° C. for 5 h. The cooled reaction was partitioned between EtOAc and brine, the aqueous layer extracted with EtOAc and the combined organics were evaporated to dryness in vacuo. The residue was purified by column chromatography on silica gel eluting with EtOAc/heptanes (50/50 to 90/10) to afford phenyl 7-cyclobutoxy-2-(1-methyl-2-oxabicyclo[2.2.2]octan-4-yl)imidazo[1,2-a]pyridine-6-carboxylate as a pale-yellow oil, 615 mg 81.0% yield. LCMS m/z=433.2 [M+H]⁺

### Preparation 272: Phenyl 7-(difluoromethoxy)-2-(1-methyl-2-oxabicyclo[2.1.1]hexan-4-yl)imidazo[1,2-a]pyridine-6-carboxylate

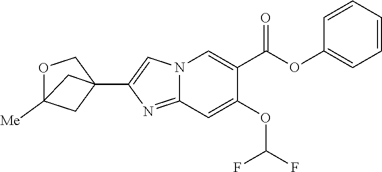 [SMILES: CC12CC(c3cn4cc(C(=O)Oc5ccccc5)c(OC(F)F)cc4n3)(CO1)C2]

TEA (176.1 mg, 1.74 mmol) was added to a mixture of 6-bromo-7-(difluoromethoxy)-2-(1-methyl-2-oxabicyclo[2.1.1]hexan-4-yl)imidazo[1,2-a]pyridine (Preparation 230, 250 mg, 0.696 mmol), Pd(OAc)₂ (4.7 mg, 0.021 mmol), Xantphos (24.1 mg, 0.042 mmol) and phenyl formate (212.5 mg, 1.74 mmol) in MeCN (2.78 mL) at rt in a closed vial, and the reaction heated at 80° C. overnight. The cooled mixture was dry loaded onto silica gel and purified by column chromatography eluting with (0-40% heptanes/3:1 EtOAc:EtOH), to afford phenyl 7-(difluoromethoxy)-2-(1-methyl-2-oxabicyclo[2.1.1]hexan-4-yl)imidazo[1,2-a]pyridine-6-carboxylate (210 mg, 75.3% yield). LCMS m/z=401.2 [M+H]⁺

### Preparations 273 to 280

The compounds in the following table were prepared from the appropriate bromide and phenyl formate following a similar procedure to that described in Preparation 272.

| Prep. no | Structure/Name/Starting materials (SM)/Yield/Data |
|---|---|
| 273 | 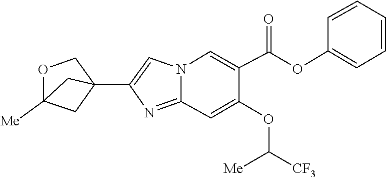 [SMILES: CC(Oc1cc2nc(C34COC(C)(C3)C4)cn2cc1C(=O)Oc1ccccc1)C(F)(F)F] |
| | Phenyl 2-(1-methyl-2-oxabicyclo[2.1.1]hexan-4-yl)-7-((1,1,1-trifluoropropan- |
| | 2-yl)oxy)imidazo[1,2-a]pyridine-6-carboxylate SM: 6-bromo-2-(1-methyl-2- |
| | oxabicyclo[2.1.1]hexan-4-yl)-7-((1,1,1-trifluoropropan-2-yl)oxy)imidazo[1,2- |
| | a]pyridine (Preparation 231) 300 mg, 77.8% yield. LCMS m/z = 447.1 |
| | [M + H]⁺ |
| 274 | 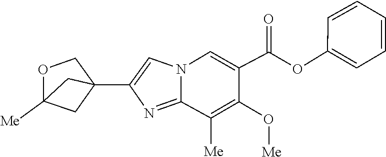 [SMILES: COc1c(C(=O)Oc2ccccc2)cn2cc(C34COC(C)(C3)C4)nc2c1C] |
| | phenyl 7-methoxy-8-methyl-2-(1-methyl-2-oxabicyclo[2.1.1]hexan-4- |
| | yl)imidazo[1,2-a]pyridine-6-carboxylate SM: 6-bromo-7-methoxy-8-methyl- |
| | 2-(1-methyl-2-oxabicyclo[2.1.1]hexan-4-yl)imidazo[1,2-a]pyridine |
| | (Preparation 232) 350 mg, 94.5% yield. LCMS m/z = 379.2 [M + H]⁺ |
| 275 | 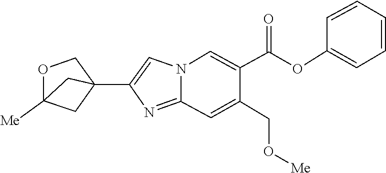 [SMILES: COCc1cc2nc(C34COC(C)(C3)C4)cn2cc1C(=O)Oc1ccccc1] |
| | phenyl 7-(methoxymethyl)-2-(1-methyl-2-oxabicyclo[2.1.1]hexan-4- |
| | yl)imidazo[1,2-a]pyridine-6-carboxylate SM: 6-bromo-7-(methoxymethyl)-2- |
| | (1-methyl-2-oxabicyclo[2.1.1]hexan-4-yl)imidazo[1,2-a]pyridine (Preparation |
| | 233) 480 mg, 82.4% yield. LCMS m/z = 379.2 [M + H]⁺ |
| 276 | 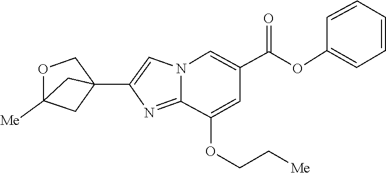 [SMILES: CCCOc1cc(C(=O)Oc2ccccc2)cn2cc(C34COC(C)(C3)C4)nc12] |
| | phenyl 2-(1-methyl-2-oxabicyclo[2.1.1]hexan-4-yl)-8-propoxyimidazo[1,2- |
| | a]pyrazine-6-carboxylate SM: 6-bromo-2-(1-methyl-2- |
| | oxabicyclo[2.1.1]hexan-4-yl)-8-propoxyimidazo[1,2-a]pyrazine (Preparation |
| | 235) 350 mg, 62.6% yield. LCMS m/z = 394.3 [M + H]⁺ |
| 277 | 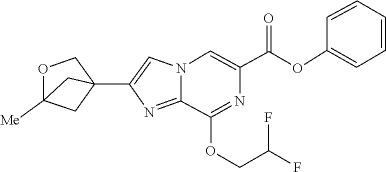 [SMILES: CC12CC(c3cn4cc(C(=O)Oc5ccccc5)nc(OCC(F)F)c4n3)(CO1)C2] |
| | phenyl 8-(2,2-difluoroethoxy)-2-(1-methyl-2-oxabicyclo[2.1.1]hexan-4- |
| | yl)imidazo[1,2-a]pyrazine-6-carboxylate SM: 6-bromo-8-(2,2- |
| | difluoroethoxy)-2-(1-methyl-2-oxabicyclo[2.1.1]hexan-4-yl)imidazo[1,2- |
| | a]pyrazine (Preparation 237) 180 mg, 73.7% yield. LCMS m/z = 416.3 |
| | [M + H]⁺ |
| 278 | 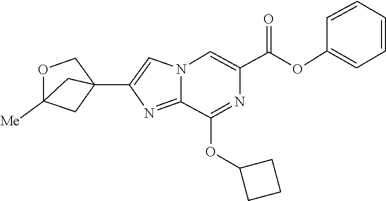 [SMILES: CC12CC(c3cn4cc(C(=O)Oc5ccccc5)nc(OC5CCC5)c4n3)(CO1)C2] |
| | Phenyl 8-cyclobutoxy-2-(1-methyl-2-oxabicyclo[2.1.1]hexan-4- |
| | yl)imidazo[1,2-a]pyrazine-6-carboxylate SM: 6-bromo-8-cyclobutoxy-2-(1- |
| | methyl-2-oxabicyclo[2.1.1]hexan-4-yl)imidazo[1,2-a]pyrazine (Preparation |
| | 238) 170 mg, 76.3% yield. LCMS m/z = 406.2 [M + H]⁺ |
| 279 | 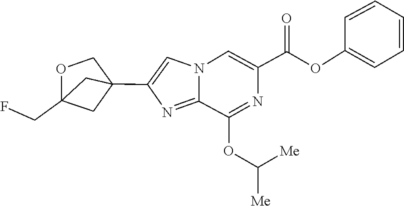 [SMILES: CC(C)Oc1nc(C(=O)Oc2ccccc2)cn2cc(C34COC(CF)(C3)C4)nc12] |
| | phenyl 2-(1-(fluoromethyl)-2-oxabicyclo[2.1.1]hexan-4-yl)-8- |
| | isopropoxyimidazo[1,2-a]pyrazine-6-carboxylate SM: 6-bromo-2-(1- |
| | (fluoromethyl)-2-oxabicyclo[2.1.1]hexan-4-yl)-8-isopropoxyimidazo[1,2- |
| | a]pyrazine (Preparation 229) 270 mg, 81.0% yield. LCMS m/z = 412.2 |
| | [M + H]⁺ |
| 280 | 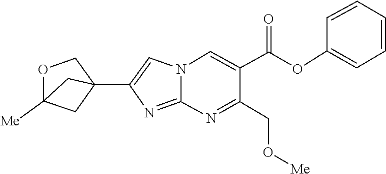 [SMILES: COCc1nc2nc(C34COC(C)(C3)C4)cn2cc1C(=O)Oc1ccccc1] |
| | phenyl 7-(methoxymethyl)-2-(1-methyl-2-oxabicyclo[2.1.1]hexan-4- |
| | yl)imidazo[1,2-a]pyrimidine-6-carboxylate SM: 6-bromo-7-(methoxymethyl)- |
| | 2-(1-methyl-2-oxabicyclo[2.1.1]hexan-4-yl)imidazo[1,2-a]pyrimidine |
| | (Preparation 234) 100 mg, 68.5% yield. LCMS m/z = 380.2 [M + H]⁺ |

### Preparation 281: Phenyl 7-(cyclopentyloxy)-2-(1-methyl-2-oxabicyclo[2.1.1]hexan-4-yl)imidazo[1,2-a]pyridine-6-carboxylate

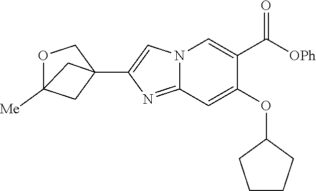 [SMILES: CC12CC(c3cn4cc(C(=O)Oc5ccccc5)c(OC5CCCC5)cc4n3)(CO1)C2]

XantPhos-Pd-G3 (23.2 mg, 0.022 mmol) was added to a mixture of 6-bromo-7-(cyclopentyloxy)-2-(1-methyl-2-oxabicyclo[2.1.1]hexan-4-yl)imidazo[1,2-a]pyridine (Preparation 226, 282 mg, 0.747 mmol), phenyl formate (469 mg, 3.84 mmol) and TEA (151 mg, 1.49 mmol) in MeCN (7.5 mL), the mixture degassed with N₂ and warmed to 90° C. overnight. The cooled reaction was diluted with water and extracted with EtOAc. The combined organic extracts were dried and evaporated under reduced pressure to afford phenyl 7-(cyclopentyloxy)-2-(1-methyl-2-oxabicyclo[2.1.1]hexan-4-yl)imidazo[1,2-a]pyridine-6-carboxylate (250 mg, 80%). LCMS m/z=419.2 [M+H]⁺.

### Preparation 282 to 285

The following compounds were prepared from the appropriate bromide and phenyl formate following a similar procedure to that described in Preparation 281.

| Prep. No | Structure/Name/Starting Material (SM)/Data |
|---|---|
| 282 | 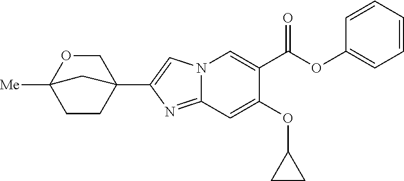 [SMILES: CC12CCC(c3cn4cc(C(=O)Oc5ccccc5)c(OC5CC5)cc4n3)(CO1)C2] |
| | phenyl 7-cyclopropoxy-2-(1-methyl-2-oxabicyclo[2.2.1]heptan-4- |
| | yl)imidazo[1,2-a]pyridine-6-carboxylate SM: 6-bromo-7-cyclopropoxy-2- |
| | (1-methyl-2-oxabicyclo[2.2.1]heptan-4-yl)imidazo[1,2-a]pyridine |
| | (Preparation 227) LCMS m/z = 405.2 [M + H]⁺ |
| 283 | 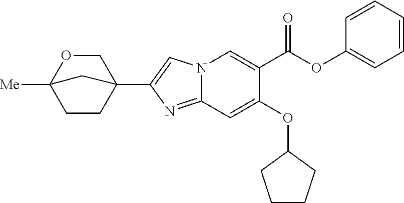 [SMILES: CC12CCC(c3cn4cc(C(=O)Oc5ccccc5)c(OC5CCCC5)cc4n3)(CO1)C2] |
| | phenyl 7-cyclopentyloxy-2-(1-methyl-2-oxabicyclo[2.2.1]heptan-4- |
| | yl)imidazo[1,2-a]pyridine-6-carboxylate SM: 6-bromo-7-(cyclopentyloxy)- |
| | 2-(1-methyl-2-oxabicyclo[2.2.1]heptan-4-yl)imidazo[1,2-a]pyridine |
| | (Preparation 228) LCMS m/z = 433.0 [M + H]⁺ |
| 284 | 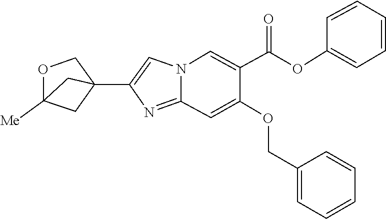 [SMILES: CC12CC(c3cn4cc(C(=O)Oc5ccccc5)c(OCc5ccccc5)cc4n3)(CO1)C2] |
| | phenyl 7-(benzyloxy)-2-(1-methyl-2-oxabicyclo[2.1.1]hexan-4- |
| | yl)imidazo[1,2-a]pyridine-6-carboxylate SM: 7-(benzyloxy)-6-bromo-2-(1- |
| | methyl-2-oxabicyclo[2.1.1]hexan-4-yl)imidazo[1,2-a]pyridine (Preparation |
| | 240) LCMS m/z = 441.1 [M + H]⁺ |
| 285 | 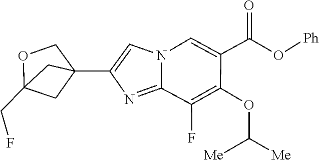 [SMILES: CC(C)Oc1c(C(=O)Oc2ccccc2)cn2cc(C34COC(CF)(C3)C4)nc2c1F] |
| | phenyl 8-fluoro-2-(1-(fluoromethyl)-2-oxabicyclo[2.1.1]hexan-4-yl)-7- |
| | isopropoxyimidazo[1,2-a]pyridine-6-carboxylate SM: 6-bromo-8-fluoro-2- |
| | (1-(fluoromethyl)-2-oxabicyclo[2.1.1]hexan-4-yl)-7-isopropoxyimidazo[1,2- |
| | a]pyridine (Preparation 241) LCMS m/z = 429.0 [M + H]⁺ |

### Preparation 286: Methyl 7-cyclopropoxy-2-(1-methyl-2-oxabicyclo[2.1.1]hexan-4-yl)imidazo[1,2-a]pyrimidine-6-carboxylate

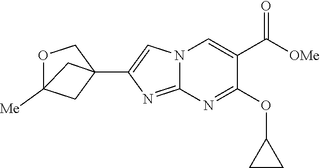 [SMILES: COC(=O)c1cn2cc(C34COC(C)(C3)C4)nc2nc1OC1CC1]

m-Tolyl formate (51.6 mg, 0.423 mmol) was added to a mixture of 6-bromo-7-cyclopropoxy-2-(1-methyl-2-oxabicyclo[2.1.1]hexan-4-yl)imidazo[1,2-a]pyrimidine (Preparation 223, 74 mg, 0.211 mmol), Xantphos Pd G3 (10.9 mg, 10.57 μmol) and TEA (42.7 mg, 0.423 mmol) in MeCN (528 μL) and the reaction heated at 80° C. overnight. The cooled mixture was concentrated in vacuo and the residue dissolved in MeOH (2.09 mL) and NaHCO₃ (176 mg, 2.09 mmol) added. The mixture was heated at 45° C. overnight, cooled to rt, filtered and concentrated in vacuo. The crude was purified by column chromatography on silica gel to afford methyl 7-cyclopropoxy-2-(1-methyl-2-oxabicyclo[2.1.1]hexan-4-yl)imidazo[1,2-a]pyrimidine-6-carboxylate, (26.0 mg, 37.7% yield). LCMS m/z=330.0 [M+H]⁺

### Preparation 287: Methyl 7-(benzyloxy)-2-(tert-butyl)imidazo[1,2-a]pyridine-6-carboxylate

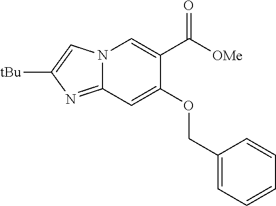 [SMILES: COC(=O)c1cn2cc(C(C)(C)C)nc2cc1OCc1ccccc1]

7-(Benzyloxy)-6-bromo-2-(tert-butyl)imidazo[1,2-a]pyridine (Preparation 239, 8.73 g, 24.30 mmol) was dissolved in MeCN (243 mL), phenyl formate (5.94 g, 48.60 mmol), followed by XantPhos-Pd-G3 (1.00 g, 0.97 mmol) and TEA (4.92 g, 48.60 mmol) were added, the mixture purged with N₂, sealed and heated at 80° C. for 2 h. The cooled reaction was diluted with water and extracted with EtOAc, the organic phase was washed with brine, dried over Na₂SO₄ and filtered. The filtrate was concentrated in vacuo and the crude was purified by silica gel column chromatography (0-100% EtOAc:Heptanes) to give phenyl 7-(benzyloxy)-2-(tert-butyl)imidazo[1,2-a]pyridine-6-carboxylate. This was treated with MeOH and K₂CO₃ and the mixture stirred at 50° C. overnight. The mixture was filtered and the filtrate evaporated under reduced pressure to afford methyl 7-(benzyloxy)-2-(tert-butyl)imidazo[1,2-a]pyridine-6-carboxylate. LCMS m/z=339.0 [M+H]⁺

### Preparation 288: Methyl 2-(tert-butyl)-7-hydroxyimidazo[1,2-a]pyridine-6-carboxylate

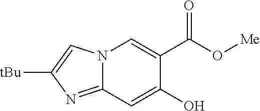 [SMILES: COC(=O)c1cn2cc(C(C)(C)C)nc2cc1O]

Pd/C (1.92 g, 1.80 mmol, 10% purity) followed by ammonium formate (11.37 g, 180.3 mmol) were added to a solution of methyl 7-(benzyloxy)-2-(tert-butyl)imidazo[1,2-a]pyridine-6-carboxylate (Preparation 287, 6.10 g, 18.03 mmol) in MeOH (180.3 mL) and the reaction stirred at 50° C. under N₂ for 10 mins. The cooled reaction mixture was filtered through Celite®, and the filtrate evaporated under reduced pressure to afford methyl 2-(tert-butyl)-7-hydroxyimidazo[1,2-a]pyridine-6-carboxylate. LCMS m/z=249.0 [M+H]⁺

### Preparation 289: Methyl 7-hydroxy-2-(1-methyl-2-oxabicyclo[2.1.1]hexan-4-yl)imidazo[1,2-a]pyridine-6-carboxylate

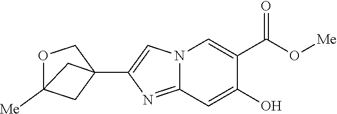 [SMILES: COC(=O)c1cn2cc(C34COC(C)(C3)C4)nc2cc1O]

was obtained from phenyl 7-(benzyloxy)-2-(1-methyl-2-oxabicyclo[2.1.1]hexan-4-yl)imidazo[1,2-a]pyridine-6-carboxylate (Preparation 284) following the procedure described in Preparation 288. LCMS m/z=289.1 [M+H]⁺

### Preparation 290: Methyl 2-(tert-butyl)-7-cyclobutoxyimidazo[1,2-a]pyridine-6-carboxylate

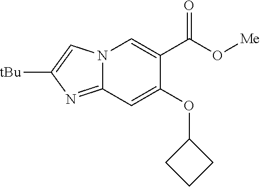 [SMILES: COC(=O)c1cn2cc(C(C)(C)C)nc2cc1OC1CCC1]

PPh₃ (on solid support, 403.1 mg, 1.54 mmol), cyclobutanol (111.5 mg, 1.55 mmol) and DIAD (293.2 mg, 1.45 mmol) were added sequentially to a solution of methyl 2-(tert-butyl)-7-hydroxyimidazo[1,2-a]pyridine-6-carboxylate (Preparation 288, 240 mg, 0.97 mmol) in THF (10 mL) and the reaction stirred for 2 h at rt. The mixture was filtered and concentrated in vacuo. The crude was purified by column chromatography on silica gel eluting with 0-65% EtOAc/heptanes to afford methyl 2-(tert-butyl)-7-cyclobutoxyimidazo[1,2-a]pyridine-6-carboxylate. LCMS m/z=303.0 [M+H]⁺

### Preparation 291: Methyl 7-((4-oxaspiro[2.4]heptan-6-yl)oxy)-2-(tert-butyl)imidazo[1,2-a]pyridine-6-carboxylate

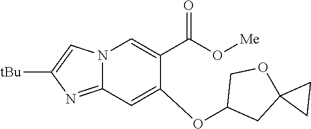 [SMILES: COC(=O)c1cn2cc(C(C)(C)C)nc2cc1OC1COC2(CC2)C1]

was obtained from methyl 2-(tert-butyl)-7-hydroxyimidazo[1,2-a]pyridine-6-carboxylate (Preparation 288) and 4-oxaspiro[2.4]heptan-6-ol, following the procedure described in Preparation 290. LCMS m/z=345.0 [M+H]⁺

### Preparation 292: Methyl 2-(1-methyl-2-oxabicyclo[2.1.1]hexan-4-yl)-7-(3-methylcyclobutoxy)imidazo[1,2-a]pyridine-6-carboxylate

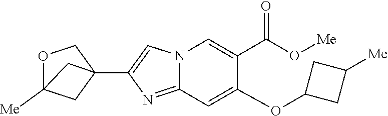 [SMILES: COC(=O)c1cn2cc(C34COC(C)(C3)C4)nc2cc1OC1CC(C)C1]

3-Methylcyclobutanol (32 μL, 0.693 mmol) was added to a solution of PPh₃ (146 mg, 0.554 mmol) and DIAD (112 mg, 0.554 mmol) in THF (3.47 mL) and the solution stirred for 10 mins at rt. Methyl 7-hydroxy-2-(1-methyl-2-oxabicyclo[2.1.1]hexan-4-yl)imidazo[1,2-a]pyridine-6-carboxylate (Preparation 289, 100 mg, 0.347 mmol) was added and the reaction stirred at rt overnight. The reaction was diluted with water, extracted with EtOAc and the combined organic extracts evaporated under reduced pressure. The crude was purified by column chromatography on silica gel eluting with EtOAc/heptanes (0/100 to 100/0) to afford methyl 2-(1-methyl-2-oxabicyclo[2.1.1]hexan-4-yl)-7-(3-methylcyclobutoxy)imidazo[1,2-a]pyridine-6-carboxylate. LCMS m/z=357.0 [M+H]⁺

### Preparation 293: Methyl 2-(1-methyl-2-oxabicyclo[2.1.1]hexan-4-yl)-7-(spiro[2.3]hexan-5-yloxy)imidazo[1,2-a]pyridine-6-carboxylate

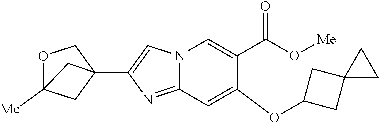 [SMILES: COC(=O)c1cn2cc(C34COC(C)(C3)C4)nc2cc1OC1CC2(CC2)C1]

was obtained from spiro[2.3]hexan-5-ol and methyl 7-hydroxy-2-(1-methyl-2-oxabicyclo[2.1.1]hexan-4-yl)imidazo[1,2-a]pyridine-6-carboxylate (Preparation 289) following the procedure described in Preparation 292. LCMS m/z=369.2 [M+H]⁺

### Preparation 294: Methyl (S)-7-(sec-butoxy)-2-(1-methyl-2-oxabicyclo[2.1.1]hexan-4-yl)imidazo[1,2-a]pyridine-6-carboxylate

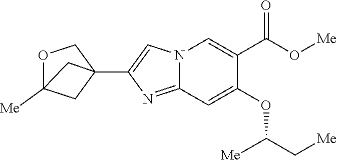 [SMILES: CC[C@H](C)Oc1cc2nc(C34COC(C)(C3)C4)cn2cc1C(=O)OC]

was obtained from (2R)-butan-2-ol and methyl 7-hydroxy-2-(1-methyl-2-oxabicyclo[2.1.1]hexan-4-yl)imidazo[1,2-a]pyridine-6-carboxylate (Preparation 289), following the procedure described in Preparation 292. LCMS m/z=345.2 [M+H]⁺

### Preparation 295: Methyl (R)-7-(sec-butoxy)-2-(1-methyl-2-oxabicyclo[2.1.1]hexan-4-yl)imidazo[1,2-a]pyridine-6-carboxylate

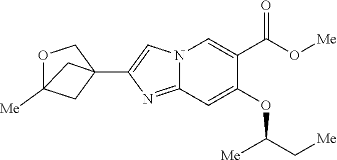 [SMILES: CC[C@@H](C)Oc1cc2nc(C34COC(C)(C3)C4)cn2cc1C(=O)OC]

was obtained from (2S)-butan-2-ol and methyl 7-hydroxy-2-(1-methyl-2-oxabicyclo[2.1.1]hexan-4-yl)imidazo[1,2-a]pyridine-6-carboxylate (Preparation 289) following the procedure described in Preparation 292. LCMS m/z=345.2 [M+H]⁺

### Preparation 296: 7-Cyclobutoxy-2-(1-methyl-2-oxabicyclo[2.2.1]heptan-4-yl)imidazo[1,2-a]pyridine-6-carboxylic acid

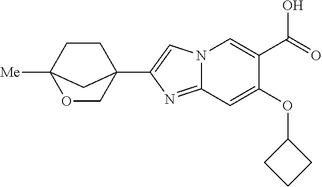 [SMILES: CC12CCC(c3cn4cc(C(=O)O)c(OC5CCC5)cc4n3)(CO1)C2]

NaOH (1 M, 2.36 mL) was added to a solution of phenyl 7-cyclobutoxy-2-(1-methyl-2-oxabicyclo[2.2.1]heptan-4-yl)imidazo[1,2-a]pyridine-6-carboxylate (Preparation 270, 493 mg, 1.18 mmol) in MeOH (2 mL) and THF (2 mL) and the mixture stirred at 40° C. for 4.5 h. The reaction mixture was neutralised by the addition of 1N HCl (2.36 mL) and the resulting clear solution concentrated and lyophilized over 72 h to afford 7-cyclobutoxy-2-(1-methyl-2-oxabicyclo[2.2.1]heptan-4-yl)imidazo[1,2-a]pyridine-6-carboxylic acid as an off-white solid. LCMS m/z=343.1 [M+H]⁺

### Preparation 297: 7-Cyclobutoxy-2-(1-methyl-2-oxabicyclo[2.2.2]octan-4-yl)imidazo[1,2-a]pyridine-6-carboxylic acid

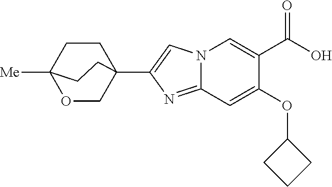 [SMILES: CC12CCC(c3cn4cc(C(=O)O)c(OC5CCC5)cc4n3)(CC1)CO2]

To a solution of phenyl 7-cyclobutoxy-2-(1-methyl-2-oxabicyclo[2.2.2]octan-4-yl)imidazo[1,2-a]pyridine-6-carboxylate (Preparation 271, 587 mg, 1.36 mmol) in MeOH (2 mL) and THF (2 mL) was added NaOH (1 M, 2.80 mL) and the reaction stirred at rt for 4.5 h. The reaction was neutralized using 1N HCl (2.8 mL), the resulting mixture was concentrated and lyophilized overnight to afford 7-cyclobutoxy-2-(1-methyl-2-oxabicyclo[2.2.2]octan-4-yl)imidazo[1,2-a]pyridine-6-carboxylic acid, 668 mg as an off-white solid. LCMS m/z=357.2 [M+H]⁺

### Preparation 298: 7-Isopropoxy-2-(1-methyl-2-oxabicyclo[2.2.1]heptan-4-yl)imidazo[1,2-a]pyridine-6-carboxylic acid

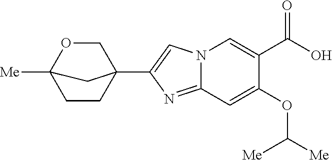 [SMILES: CC(C)Oc1cc2nc(C34CCC(C)(C3)OC4)cn2cc1C(=O)O]

A mixture of methyl 7-isopropoxy-2-(1-methyl-2-oxabicyclo[2.2.1]heptan-4-yl)imidazo[1,2-a]pyridine-6-carboxylate (Preparation 256, 542 mg, 1.57 mmol) and 1M NaOH (3.15 mL) in THF (3 mL) and MeOH (3 mL) was stirred at rt for 1.5 h. The mixture was acidified to pH 3 using 1N HCl, concentrated in vacuo and the residue lyophilised to afford 7-isopropoxy-2-(1-methyl-2-oxabicyclo[2.2.1]heptan-4-yl)imidazo[1,2-a]pyridine-6-carboxylic acid, 680 mg. LCMS m/z=331.1 [M+H]⁺

### Preparation 298AL Lithium 7-isopropoxy-2-(1-methyl-2-oxabicyclo[2.2.1]heptan-4-yl)imidazo[1,2-a]pyridine-6-carboxylate

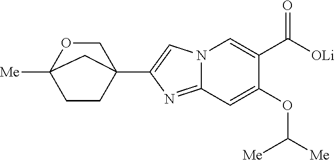

A mixture of methyl 7-isopropoxy-2-(1-methyl-2-oxabicyclo[2.2.1]heptan-4-yl)imidazo[1,2-a]pyridine-6-carboxylate (Preparation 256, 8.10 g, 23.52 mmol), LiOH.H₂O (987 mg, 23.52 mmol), THF (100 mL) and H₂O (10 mL) was stirred at rt for 16 h. The solvent was evaporated in vacuo and the residue crystallized from THF/hexane (20/100 mL) to afford lithium 7-isopropoxy-2-(1-methyl-2-oxabicyclo[2.2.1]heptan-4-yl)imidazo[1,2-a]pyridine-6-carboxylate (7.20 g, 85%). LCMS m/z=331.0 [M-Li+H]⁺.

### Preparation 299: 2-Cyclopropyl-7-isopropoxyimidazo[1,2-a]pyridine-6-carboxylic acid

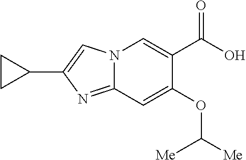 [SMILES: CC(C)Oc1cc2nc(C3CC3)cn2cc1C(=O)O]

A mixture of methyl 2-cyclopropyl-7-isopropoxyimidazo[1,2-a]pyridine-6-carboxylate (Preparation 254, 14 g, 51.04 mmol), NaOH (3.06 g, 76.56 mmol), MeOH (50 mL) and H₂O (100 mL) was stirred at 40° C. for 16 h. The mixture was concentrated in vacuo, the residue diluted with H₂O (100 mL), the mixture treated with activated carbon (2 g) and then filtered. The filtrate was acidified with c. HCl to pH 4-5 and evaporated to dryness in vacuo and the residue azeotroped with EtOH (100 mL). The residue was crystallized from MeCN (150 mL) to afford 2-cyclopropyl-7-isopropoxyimidazo[1,2-a]pyridine-6-carboxylic acid as a yellow solid (6.30 g, 43%). LCMS m/z=261.4 [M+H]⁺

### Preparation 300: 7-(Benzyloxy)-2-cyclopropylimidazo[1,2-a]pyridine-6-carboxylic acid

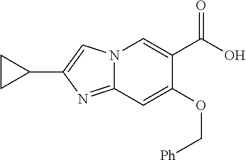 [SMILES: O=C(O)c1cn2cc(C3CC3)nc2cc1OCc1ccccc1]

A mixture of methyl 7-(benzyloxy)-2-cyclopropylimidazo[1,2-a]pyridine-6-carboxylate (Preparation 255, 8.5 g, 26.37 mmol), NaOH (1.58 g, 39.55 mmol), H₂O (100 mL) and MeOH (50 mL) was stirred at 40° C. for 14 h. The volatiles were removed by evaporation in vacuo and H₂O (50 mL) and activated carbon (2 g) were added and the mixture immediately filtered. The filtrate was acidified with c.HCl to pH 3-4 and the precipitate collected by filtration to afford 7-(benzyloxy)-2-cyclopropylimidazo[1,2-a]pyridine-6-carboxylic acid as a white solid (7.0 g, 86%). LCMS m/z=309.0 [M+H]⁺.

### Preparations 301 to 305

To a solution of the appropriate methyl ester (1.0 equiv.) in water/MeOH (1/1 V/V) was added NaOH (3.0 equiv.) and the reaction stirred at rt for 16 h. The reaction mixture was concentrated in vacuo, the residue was diluted with water and the pH adjusted to 3 using 1M aq. HCl. The mixture was lyophilized to give the title compound.

| Prep. No | Structure/Name/ Starting Material (SM)/Yield/Data |
|---|---|
| 301 | 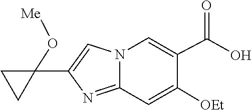 [SMILES: CCOc1cc2nc(C3(OC)CC3)cn2cc1C(=O)O] |
| | 7-ethoxy-2-(1-methoxycyclopropyl)imidazo[1,2-a]pyridine-6-carboxylic acid |
| | SM: methyl 7-ethoxy-2-(1-methoxycyclopropyl)imidazo[1,2-a]pyridine-6- |
| | carboxylate (Preparation 244) |
| | 800 mg, crude as a yellow solid. LCMS m/z = 276.9 [M + H]⁺ |
| 302 | 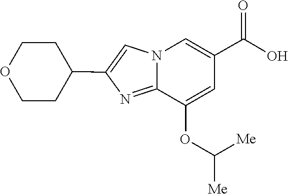 [SMILES: CC(C)Oc1cc(C(=O)O)cn2cc(C3CCOCC3)nc12] |
| | 8-isopropoxy-2-(tetrahydro-2H-pyran-4-yl)imidazo[1,2-a]pyridine-6-carboxylic |
| | acid SM: methyl 8-isopropoxy-2-(tetrahydro-2H-pyran-4-yl)imidazo[1,2- |
| | a]pyridine-6-carboxylate (Preparation 247) 600 mg, crude as a brown solid |
| 303 | 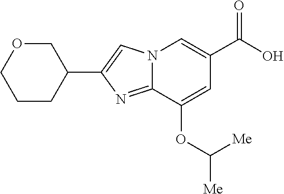 [SMILES: CC(C)Oc1cc(C(=O)O)cn2cc(C3CCCOC3)nc12] |
| | 8-isopropoxy-2-(tetrahydro-2H-pyran-3-yl)imidazo[1,2-a]pyridine-6-carboxylic |
| | acid SM: methyl 8-isopropoxy-2-(tetrahydro-2H-pyran-3-yl)imidazo[1,2- |
| | a]pyridine-6-carboxylate (Preparation 248) 1.00 g, crude, as brown solid. LCMS |
| | m/z = 305.2 [M + H]⁺ |
| 304^{A} | 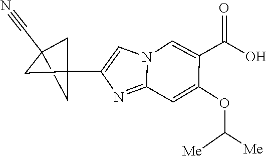 [SMILES: CC(C)Oc1cc2nc(C34CC(C#N)(C3)C4)cn2cc1C(=O)O] |
| | 2-(3-cyanobicyclo[1.1.1]pentan-1-yl)-7-isopropoxyimidazo[1,2-a]pyridine-6- |
| | carboxylic acid SM: methyl 2-(3-cyanobicyclo[1.1.1]pentan-1-yl)-7- |
| | isopropoxyimidazo[1,2-a]pyridine-6-carboxylate (Preparation 246) |
| | 120 mg, 86.8% yield as a white solid. ¹H NMR (400 MHz, MeOH-d₄) δ: 1.45 (d, |
| | 6H), 2.70 (s, 6H), 4.88 (s, 1H), 7.12 (s, 1H), 7.79 (s, 1H), 8.92 (s, 1H) |
| 305 | 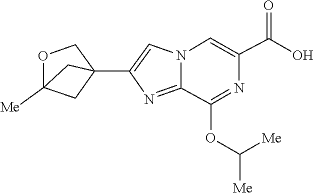 [SMILES: CC(C)Oc1nc(C(=O)O)cn2cc(C34COC(C)(C3)C4)nc12] |
| | 8-isopropoxy-2-(1-methyl-2-oxabicyclo[2.1.1]hexan-4-yl)imidazo[1,2-a]pyrazine- |
| | 6-carboxylic acid SM: methyl 8-isopropoxy-2-(1-methyl-2- |
| | oxabicyclo[2.1.1]hexan-4-yl)imidazo[1,2-a]pyrazine-6-carboxylate (Preparation |
| | 250) a brown solid, 260 mg, 87.3% yield. |
| | ¹H NMR (400 MHz, MeOH-d₄) δ: 1.45 (d, 6H), 1.50 (s, 3H), 1.88-1.90 (m, 2H), |
| | 2.11-2.16 (m, 2H), 4.04 (s, 2H), 5.74-5.91 (m, 1H), 7.85 (s, 1H), 8.55 (s, 1H). |

^{A}Solution neutralized with aq. KHSO₄, instead of HCl

### Preparation 306: 2-Chloro-7-isopropoxyimidazo[1,2-a]pyridine-6-carboxylic acid

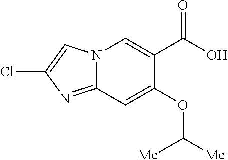 [SMILES: CC(C)Oc1cc2nc(Cl)cn2cc1C(=O)O]

Phenyl formate (158.6 mg, 1.30 mmol), XantPhos-Pd-G3 (67.1 mg, 0.065 mmol) and TEA (131.4 mg, 1.30 mmol) were added to a solution of 6-bromo-2-chloro-7-isopropoxyimidazo[1,2-a]pyridine (Preparation 213, 188 mg, 0.649 mmol) in MeCN (6.49 mL), the mixture purged with N₂ and heated at 80° C. for 2 h. The cooled reaction was diluted with water, extracted with EtOAc, the combined organic extracts washed with brine and dried over Na₂SO₄. The filtrate was evaporated under reduced pressure and the residue treated with a large excess of Na₂CO₃ in MeOH at 50° C. The mixture was filtered, the filtrate concentrated in vacuo and the crude purified by silica gel column chromatography eluting with 0-100% EtOAc-heptanes. The product (130 mg, 0.484 mmol) was dissolved in MeOH (1.0 mL), THF (4.0 mL) and water (2.0 mL), LiOH.H₂O (57.9 mg, 2.42 mmol) added and the reaction stirred at rt overnight. The mixture was acidified to pH 2 using 1N HCl aq, extracted with EtOAc and the combined organic extracts dried over Na₂SO₄, filtered and evaporated under reduced pressure. The crude was purified by reverse phase chromatography on an SCX resin column, washing with MeOH (3× column volume) and eluting with 2N NH₃ in MeOH to afford 2-chloro-7-isopropoxyimidazo[1,2-a]pyridine-6-carboxylic acid. LCMS m/z=254.9 [M+H]⁺

### Preparation 307: 2-(2-Oxabicyclo[2.1.1]hexan-4-yl)-7-isopropoxyimidazo[1,2-a]pyridine-6-carboxylic acid

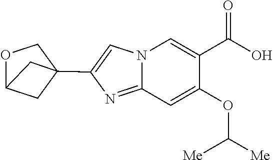 [SMILES: CC(C)Oc1cc2nc(C34COC(C3)C4)cn2cc1C(=O)O]

A mixture of methyl 2-(2-oxabicyclo[2.1.1]hexan-4-yl)-7-isopropoxyimidazo[1,2-a]pyridine-6-carboxylate (Preparation 261, 1.65 g, 5.22 mmol), LiOH.H₂O (218.8 mg, 5.22 mmol) in THF (50 mL) and water (5 mL) was stirred at rt for 16 h. The THF was removed in vacuo, H₂O (50 mL) and activated carbon (1 g) were added and the mixture filtered. The filtrate was acidified with conc. HCl to pH 3-4 and the precipitate was filtered, washed with water and air-dried to afford 2-(2-oxabicyclo[2.1.1]hexan-4-yl)-7-isopropoxyimidazo[1,2-a]pyridine-6-carboxylic acid (1.30 g, 76.4% yield) as a yellow solid. LCMS m/z=303.0 [M+H]⁺

Preparation 308: 7-(Benzyloxy)-2-(1-methyl-2-oxabicyclo[2.1.1]hexan-4-yl)imidazo[1,2-a]pyridine-6-carboxylic acid

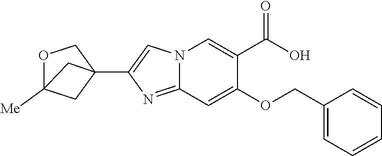 [SMILES: CC12CC(c3cn4cc(C(=O)O)c(OCc5ccccc5)cc4n3)(CO1)C2]

was obtained as a yellow solid from methyl 7-(benzyloxy)-2-(1-methyl-2-oxabicyclo[2.1.1]hexan-4-yl)imidazo[1,2-a]pyridine-6-carboxylate (Preparation 263), 11.2 g, 69% yield, following the procedure described in Preparation 307. LCMS m/z=365.0 [M+H]⁺

### Preparation 309: 7-Isopropoxy-2-(1-methyl-2-oxabicyclo[2.2.2]octan-4-yl)imidazo[1,2-a]pyridine-6-carboxylic acid

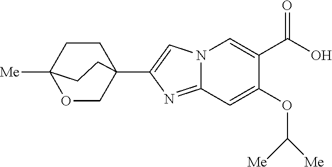 [SMILES: CC(C)Oc1cc2nc(C34CCC(C)(CC3)OC4)cn2cc1C(=O)O]

A mixture of methyl 7-isopropoxy-2-(1-methyl-2-oxabicyclo[2.2.2]octan-4-yl)imidazo[1,2-a]pyridine-6-carboxylate (Preparation 257, 400 mg, 1.12 mmol) and LiOH.H₂O (94 mg, 2.23 mmol) in MeOH (0.55 mL), THF (4 mL) and water (1 mL) was stirred at rt overnight. The mixture was neutralized with 4 M HCl in dioxane and evaporated to dryness in vacuo and dried under high vacuum to afford 7-isopropoxy-2-(1-methyl-2-oxabicyclo[2.2.2]octan-4-yl)imidazo[1,2-a]pyridine-6-carboxylic acid (384 mg, crude). LCMS m/z=345.2 [M+H]⁺

### Preparation 310: 7-(Cyclopentyloxy)-2-(1-methyl-2-oxabicyclo[2.1.1]hexan-4-yl)imidazo[1,2-a]pyridine-6-carboxylic acid

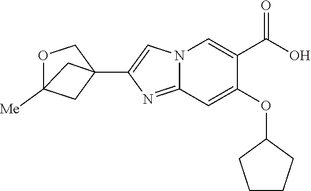 [SMILES: CC12CC(c3cn4cc(C(=O)O)c(OC5CCCC5)cc4n3)(CO1)C2]

LiOH.H₂O (71.5 mg, 2.99 mmol) was added to a mixture of phenyl 7-(cyclopentyloxy)-2-(1-methyl-2-oxabicyclo[2.1.1]hexan-4-yl)imidazo[1,2-a]pyridine-6-carboxylate (Preparation 281, 250 mg, 0.597 mmol) in THF (4 mL), MeOH (1 mL) and water (1 mL) and the mixture was stirred at rt overnight. The reaction mixture was acidified to approx. pH=2 with 1N HCl and evaporated to dryness. The residue was purified by SCX ion exchange column to afford 7-(cyclopentyloxy)-2-(1-methyl-2-oxabicyclo[2.1.1]hexan-4-yl)imidazo[1,2-a]pyridine-6-carboxylic acid (205 mg, 100%). LCMS m/z=343.2 [M+H]⁺

### Preparation 311: 2-(1-(Fluoromethyl)-2-oxabicyclo[2.1.1]hexan-4-yl)-7-isopropoxyimidazo[1,2-a]pyrimidine-6-carboxylic acid

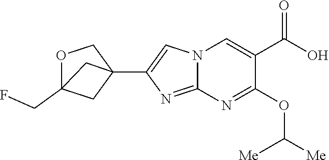 [SMILES: CC(C)Oc1nc2nc(C34COC(CF)(C3)C4)cn2cc1C(=O)O]

A mixture of isopropyl 2-(1-(fluoromethyl)-2-oxabicyclo[2.1.1]hexan-4-yl)-7-isopropoxyimidazo[1,2-a]pyrimidine-6-carboxylate (Preparation 267, 170 mg, 0.450 mmol) and LiOH.H₂O (37.8 mg, 0.901 mmol) in MeOH (0.45 mL), THF (3.2 mL) and water (0.8 mL) was stirred at rt overnight. The mixture was neutralized with 4 M HCl in dioxane and evaporated to dryness in vacuo and dried under high vacuum to afford 2-(1-(fluoromethyl)-2-oxabicyclo[2.1.1]hexan-4-yl)-7-isopropoxyimidazo[1,2-a]pyrimidine-6-carboxylic acid (151 mg, assumed 100%). LCMS m/z=336.1 [M+H]⁺

### Preparation 312: 7-Isopropoxy-2-(4-methyl-2-oxabicyclo[2.2.2]octan-1-yl)imidazo[1,2-a]pyrimidine-6-carboxylic acid

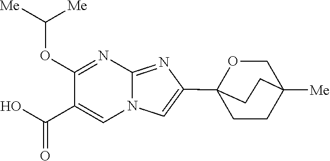 [SMILES: CC(C)Oc1nc2nc(C34CCC(C)(CC3)CO4)cn2cc1C(=O)O]

A mixture of isopropyl 7-isopropoxy-2-(4-methyl-2-oxabicyclo[2.2.2]octan-1-yl)imidazo[1,2-a]pyrimidine-6-carboxylate (Preparation 268, 700 mg, 1.81 mmol) and LiOH.H₂O (152 mg, 3.61 mmol) in MeOH (0.3 mL), THF (2 mL) and H₂O (0.5 mL) was stirred at rt overnight. The mixture was neutralized with 4 M HCl in dioxane and evaporated to dryness in vacuo to afford 7-isopropoxy-2-(4-methyl-2-oxabicyclo[2.2.2]octan-1-yl)imidazo[1,2-a]pyrimidine-6-carboxylic acid (634 mg, assumed 100%). LCMS m/z=346.1 [M+H]t

Preparation 313: 7-Cyclobutoxy-2-(1-methyl-2-oxabicyclo[2.1.1]hexan-4-yl)imidazo[1,2-a]pyrimidine-6-carboxylic acid

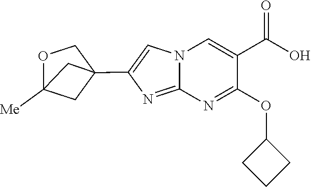 [SMILES: CC12CC(c3cn4cc(C(=O)O)c(OC5CCC5)nc4n3)(CO1)C2]

LiOH.H₂O (59.2 mg, 2.47 mmol) was added to a solution of methyl 7-cyclobutoxy-2-(1-methyl-2-oxabicyclo[2.1.1]hexan-4-yl)imidazo[1,2-a]pyrimidine-6-carboxylate (Preparation 243, 283 mg, 0.824 mmol) in MeOH (3 mL) and water (3 mL) and the reaction stirred at 25° C. for 16 h. The mixture was diluted with saturated HCl aq. to pH=7, then concentrated in vacuo. The residue was co-evaporated with toluene to afford 7-cyclobutoxy-2-(1-methyl-2-oxabicyclo[2.1.1]hexan-4-yl)imidazo[1,2-a]pyrimidine-6-carboxylic acid (200 mg, 67.0% yield) as black oil. LCMS m/z=330.2 [M+H]⁺

### Preparation 314: 7-(Cyclopropylmethoxy)-2-(1-methyl-2-oxabicyclo[2.1.1]hexan-4-yl)imidazo[1,2-a]pyrimidine-6-carboxylic acid

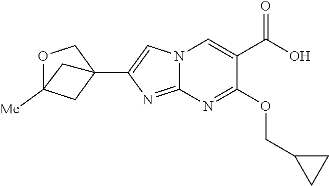 [SMILES: CC12CC(c3cn4cc(C(=O)O)c(OCC5CC5)nc4n3)(CO1)C2]

was obtained as a brown solid, 450 mg, 89.8% yield, from methyl 7-(cyclopropylmethoxy)-2-(1-methyl-2-oxabicyclo[2.1.1]hexan-4-yl)imidazo[1,2-a]pyrimidine-6-carboxylate (Preparation 251) following a similar procedure to that described in Preparation 313, except the residue was recrystallized from water. LCMS m/z=329.9 [M+H]⁺

### Preparation 315: 8-Chloro-7-isopropoxy-2-(1-methyl-2-oxabicyclo[2.1.1]hexan-4-yl)imidazo[1,2-a]pyridine-6-carboxylic acid

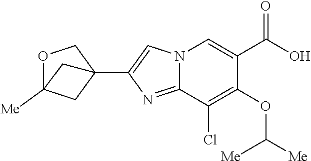 [SMILES: CC(C)Oc1c(C(=O)O)cn2cc(C34COC(C)(C3)C4)nc2c1Cl]

was obtained, 410 mg, as a brown solid, from isopropyl 8-chloro-7-isopropoxy-2-(1-methyl-2-oxabicyclo[2.1.1]hexan-4-yl)imidazo[1,2-a]pyridine-6-carboxylate (Preparation 266) following the procedure described in Preparation 313. LCMS m/z=350.9 [M+H]⁺

### Preparations 316 to 340

LiOH.H₂O (2 to 10 equiv.) was added to a solution of the appropriate ester (1 equiv.) in MeOH/THF/H₂O (1/1 to 8/1 to 2, V/V/V) and the reaction stirred at rt for 16 h. The mixture was neutralized using 4M HCl and the solution evaporated under reduced pressure to afford the desired compound.

| Prep. No | Structure/Name/Starting Material (SM)/Yield/Data |
|---|---|
| 316 | 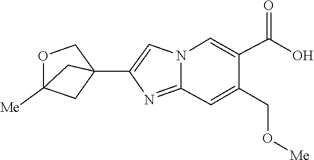 [SMILES: COCc1cc2nc(C34COC(C)(C3)C4)cn2cc1C(=O)O] |
| | 7-(methoxymethyl)-2-(1-methyl-2-oxabicyclo[2.1.1]hexan-4-yl)imidazo[1,2- |
| | a]pyridine-6-carboxylic acid SM: phenyl 7-(methoxymethyl)-2-(1-methyl-2- |
| | oxabicyclo[2.1.1]hexan-4-yl)imidazo[1,2-a]pyridine-6-carboxylate (Preparation |
| | 275) LCMS m/z = 303.1 [M + H]⁺ |
| 317 | 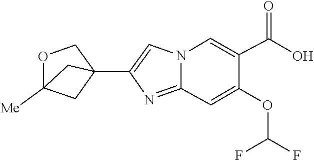 [SMILES: CC12CC(c3cn4cc(C(=O)O)c(OC(F)F)cc4n3)(CO1)C2] |
| | 7-(difluoromethoxy)-2-(1-methyl-2-oxabicyclo[2.1.1]hexan-4-yl)imidazo[1,2- |
| | a]pyridine-6-carboxylic acid SM: phenyl 7-(difluoromethoxy)-2-(1-methyl-2- |
| | oxabicyclo[2.1.1]hexan-4-yl)imidazo[1,2-a]pyridine-6-carboxylate (Preparation |
| | 272). LCMS m/z = 325.0 [M + H]⁺ |
| 318 | 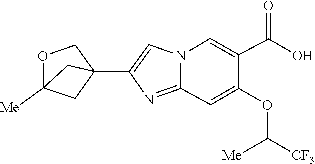 [SMILES: CC(Oc1cc2nc(C34COC(C)(C3)C4)cn2cc1C(=O)O)C(F)(F)F] |
| | 2-(1-methyl-2-oxabicyclo[2.1.1]hexan-4-yl)-7-((1,1,1-trifluoropropan-2- |
| | yl)oxy)imidazo[1,2-a]pyridine-6-carboxylic acid SM: phenyl 2-(1-methyl-2- |
| | oxabicyclo[2.1.1]hexan-4-yl)-7-((1,1,1-trifluoropropan-2-yl)oxy)imidazo[1,2- |
| | a]pyridine-6-carboxylate (Preparation 273) LCMS m/z = 371.1 [M + H]⁺ |
| 319^{A} | 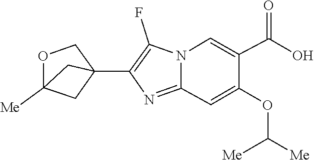 [SMILES: CC(C)Oc1cc2nc(C34COC(C)(C3)C4)c(F)n2cc1C(=O)O] |
| | 3-fluoro-7-isopropoxy-2-(1-methyl-2-oxabicyclo[2.1.1]hexan-4-yl)imidazo[1,2- |
| | a]pyridine-6-carboxylic acid SM: methyl 3-fluoro-7-isopropoxy-2-(1-methyl-2- |
| | oxabicyclo[2.1.1]hexan-4-yl)imidazo[1,2-a]pyridine-6-carboxylate (Preparation |
| | 269) LCMS m/z = 335.2 [M + H]⁺ |
| 320^{A} | 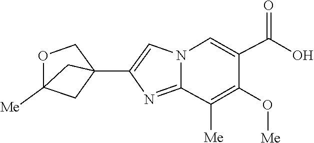 [SMILES: COc1c(C(=O)O)cn2cc(C34COC(C)(C3)C4)nc2c1C] |
| | 7-methoxy-8-methyl-2-(1-methyl-2-oxabicyclo[2.1.1]hexan-4-yl)imidazo[1,2- |
| | a]pyridine-6-carboxylic acid SM: phenyl 7-methoxy-8-methyl-2-(1-methyl-2- |
| | oxabicyclo[2.1.1]hexan-4-yl)imidazo[1,2-a]pyridine-6-carboxylate (Preparation |
| | 274) LCMS m/z = 303.1 [M + H]⁺ |
| 321 | 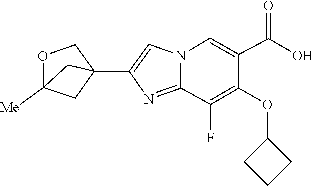 [SMILES: CC12CC(c3cn4cc(C(=O)O)c(OC5CCC5)c(F)c4n3)(CO1)C2] |
| | 7-cyclobutoxy-8-fluoro-2-(1-methyl-2-oxabicyclo[2.1.1]hexan-4-yl)imidazo[1,2- |
| | a]pyridine-6-carboxylic acid SM: methyl 7-cyclobutoxy-8-fluoro-2-(1-methyl-2- |
| | oxabicyclo[2.1.1]hexan-4-yl)imidazo[1,2-a]pyridine-6-carboxylate (Preparation |
| | 249) 259 mg, crude as a brown solid. |
| 322^{B} | 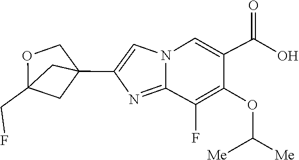 [SMILES: CC(C)Oc1c(C(=O)O)cn2cc(C34COC(CF)(C3)C4)nc2c1F] |
| | 8-fluoro-2-(1-(fluoromethyl)-2-oxabicyclo[2.1.1]hexan-4-yl)-7- |
| | isopropoxyimidazo[1,2-a]pyridine-6-carboxylic acid SM: phenyl 8-fluoro-2-(1- |
| | (fluoromethyl)-2-oxabicyclo[2.1.1]hexan-4-yl)-7-isopropoxyimidazo[1,2- |
| | a]pyridine-6-carboxylate (Preparation 285). LCMS m/z = 353.0 [M + H]⁺ |
| 323^{B} | 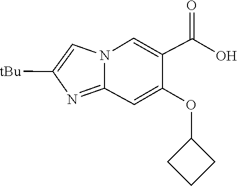 [SMILES: CC(C)(C)c1cn2cc(C(=O)O)c(OC3CCC3)cc2n1] |
| | 2-(tert-butyl)-7-cyclobutoxyimidazo[1,2-a]pyridine-6-carboxylic acid |
| | SM: methyl 2-(tert-butyl)-7-cyclobutoxyimidazo[1,2-a]pyridine-6-carboxylate |
| | (Preparation 290) LCMS m/z = 289.0 [M + H]⁺ |
| 324^{B} | 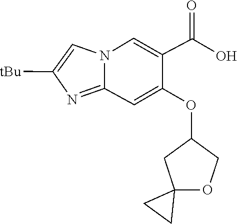 [SMILES: CC(C)(C)c1cn2cc(C(=O)O)c(OC3COC4(CC4)C3)cc2n1] |
| | 7-((4-oxaspiro[2.4]heptan-6-yl)oxy)-2-(tert-butyl)imidazo[1,2-a]pyridine-6- |
| | carboxylic acid SM: methyl 7-((4-oxaspiro[2.4]heptan-6-yl)oxy)-2-(tert- |
| | butyl)imidazo[1,2-a]pyridine-6-carboxylate (Preparation 291) LCMS m/z = 331.0 |
| | [M + H]⁺ |
| 325^{B} | 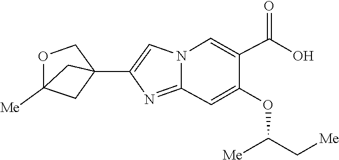 [SMILES: CC[C@H](C)Oc1cc2nc(C34COC(C)(C3)C4)cn2cc1C(=O)O] |
| | (S)-7-(sec-butoxy)-2-(1-methyl-2-oxabicyclo[2.1.1]hexan-4-yl)imidazo[1,2- |
| | a]pyridine-6-carboxylic acid SM: methyl (S)-7-(sec-butoxy)-2-(1-methyl-2- |
| | oxabicyclo[2.1.1]hexan-4-yl)imidazo[1,2-a]pyridine-6-carboxylate (Preparation |
| | 294) LCMS m/z = 331.2 [M + H]⁺ |
| 326^{B} | 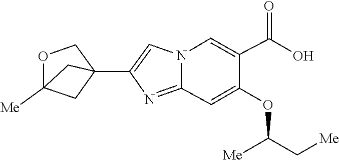 [SMILES: CC[C@@H](C)Oc1cc2nc(C34COC(C)(C3)C4)cn2cc1C(=O)O] |
| | (R)-7-(sec-butoxy)-2-(1-methyl-2-oxabicyclo[2.1.1]hexan-4-yl)imidazo[1,2- |
| | a]pyridine-6-carboxylic acid SM: methyl (R)-7-(sec-butoxy)-2-(1-methyl-2- |
| | oxabicyclo[2.1.1]hexan-4-yl)imidazo[1,2-a]pyridine-6-carboxylate (Preparation |
| | 295). LCMS m/z = 331.2 [M + H]⁺ |
| 327^{C} | 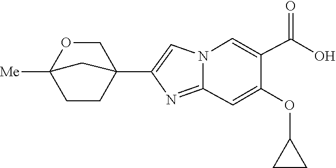 [SMILES: CC12CCC(c3cn4cc(C(=O)O)c(OC5CC5)cc4n3)(CO1)C2] |
| | 7-cyclopropoxy-2-(1-methyl-2-oxabicyclo[2.2.1]heptan-4-yl)imidazo[1,2- |
| | a]pyridine-6-carboxylic acid SM: phenyl 7-cyclopropoxy-2-(1-methyl-2- |
| | oxabicyclo[2.2.1]heptan-4-yl)imidazo[1,2-a]pyridine-6-carboxylate (Preparation |
| | 282) LCMS m/z = 329.2 [M + H]⁺ |
| 328 | 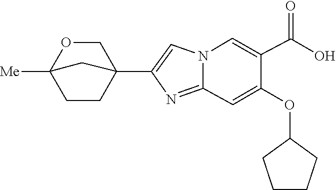 [SMILES: CC12CCC(c3cn4cc(C(=O)O)c(OC5CCCC5)cc4n3)(CO1)C2] |
| | 7-cyclopentyloxy-2-(1-methyl-2-oxabicyclo[2.2.1]heptan-4-yl)imidazo[1,2- |
| | a]pyridine-6-carboxylic acid SM: phenyl 7-cyclopentyloxy-2-(1-methyl-2- |
| | oxabicyclo[2.2.1]heptan-4-yl)imidazo[1,2-a]pyridine-6-carboxylate (Preparation |
| | 283) LCMS m/z = 357.2 [M + H]⁺ |
| 329 | 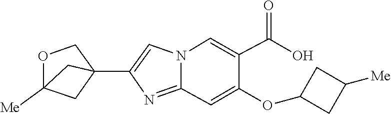 [SMILES: CC1CC(Oc2cc3nc(C45COC(C)(C4)C5)cn3cc2C(=O)O)C1] |
| | 2-(1-methyl-2-oxabicyclo[2.1.1]hexan-4-yl)-7-(3- |
| | methylcyclobutoxy)imidazo[1,2-a]pyridine-6-carboxylic acid SM: methyl 2-(1- |
| | methyl-2-oxabicyclo[2.1.1]hexan-4-yl)-7-(3-methylcyclobutoxy)imidazo[1,2- |
| | a]pyridine-6-carboxylate (Preparation 292) LCMS m/z = 343.2 [M + H]⁺ |
| 330 | 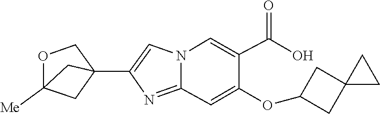 [SMILES: CC12CC(c3cn4cc(C(=O)O)c(OC5CC6(CC6)C5)cc4n3)(CO1)C2] |
| | 2-(1-methyl-2-oxabicyclo[2.1.1]hexan-4-yl)-7-(spiro[2.3]hexan-5- |
| | yloxy)imidazo[1,2-a]pyridine-6-carboxylic acid SM: methyl 2-(1-methyl-2- |
| | oxabicyclo[2.1.1]hexan-4-yl)-7-(spiro[2.3]hexan-5-yloxy)imidazo[1,2-a]pyridine- |
| | 6-carboxylate (Preparation 293) LCMS m/z = 355.2 [M + H]⁺ |
| 331 | 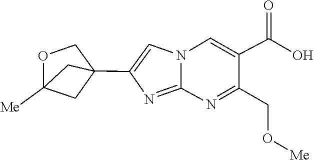 [SMILES: COCc1nc2nc(C34COC(C)(C3)C4)cn2cc1C(=O)O] |
| | 7-(methoxymethyl)-2-(1-methyl-2-oxabicyclo[2.1.1]hexan-4-yl)imidazo[1,2- |
| | a]pyrimidine-6-carboxylic acid SM: phenyl 7-(methoxymethyl)-2-(1-methyl-2- |
| | oxabicyclo[2.1.1]hexan-4-yl)imidazo[1,2-a]pyrimidine-6-carboxylate (Preparation |
| | 280) LCMS m/z = 304.1 [M + H]⁺ |
| 332^{B} | 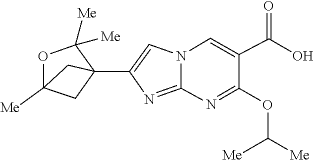 [SMILES: CC(C)Oc1nc2nc(C34CC(C)(C3)OC4(C)C)cn2cc1C(=O)O] |
| | 7-isopropoxy-2-(1,3,3-trimethyl-2-oxabicyclo[2.1.1]hexan-4-yl)imidazo[1,2- |
| | a]pyrimidine-6-carboxylic acid SM: isopropyl 7-isopropoxy-2-(1,3,3-trimethyl-2- |
| | oxabicyclo[2.1.1]hexan-4-yl)imidazo[1,2-a]pyrimidine-6-carboxylate (Prep. 265) |
| 333 | 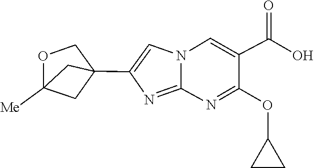 [SMILES: CC12CC(c3cn4cc(C(=O)O)c(OC5CC5)nc4n3)(CO1)C2] |
| | 7-cyclopropoxy-2-(1-methyl-2-oxabicyclo[2.1.1]hexan-4-yl)imidazo[1,2- |
| | a]pyrimidine-6-carboxylic acid SM: methyl 7-cyclopropoxy-2-(1-methyl-2- |
| | oxabicyclo[2.1.1]hexan-4-yl)imidazo[1,2-a]pyrimidine-6-carboxylate (Preparation |
| | 286) LCMS m/z = 316.0 [M + H]⁺ |
| 334 | 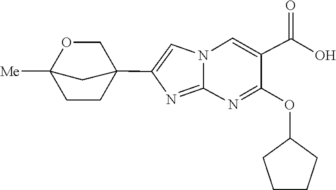 [SMILES: CC12CCC(c3cn4cc(C(=O)O)c(OC5CCCC5)nc4n3)(CO1)C2] |
| | 7-(cyclopentyloxy)-2-(1-methyl-2-oxabicyclo[2.2.1]heptan-4-yl)imidazo[1,2- |
| | a]pyrimidine-6-carboxylic acid SM: ethyl 7-(cyclopentyloxy)-2-(1-methyl-2- |
| | oxabicyclo[2.2.1]heptan-4-yl)imidazo[1,2-a]pyrimidine-6-carboxylate |
| | (Preparation 258) LCMS m/z = 358.2 [M + H]⁺ |
| 335^{A} | 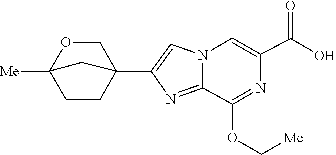 [SMILES: CCOc1nc(C(=O)O)cn2cc(C34CCC(C)(C3)OC4)nc12] |
| | 8-ethoxy-2-(1-methyl-2-oxabicyclo[2.2.1]heptan-4-yl)imidazo[1,2-a]pyrazine-6- |
| | carboxylic acid SM: methyl 8-ethoxy-2-(1-methyl-2-oxabicyclo[2.2.1]heptan-4- |
| | yl)imidazo[1,2-a]pyrazine-6-carboxylate (Preparation 264) |
| | 970 mg, 71.7%. LCMS m/z = 318.1 [M + H]⁺ |
| 336^{A} | 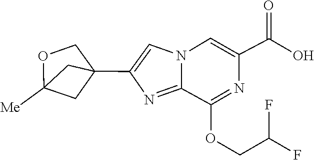 [SMILES: CC12CC(c3cn4cc(C(=O)O)nc(OCC(F)F)c4n3)(CO1)C2] |
| | 8-(2,2-difluoroethoxy)-2-(1-methyl-2-oxabicyclo[2.1.1]hexan-4-yl)imidazo[1,2- |
| | a]pyrazine-6-carboxylic acid SM: phenyl 8-(2,2-difluoroethoxy)-2-(1-methyl-2- |
| | oxabicyclo[2.1.1]hexan-4-yl)imidazo[1,2-a]pyrazine-6-carboxylate (Preparation |
| | 277) LCMS m/z = 340.2 [M + H]⁺ |
| 337^{A} | 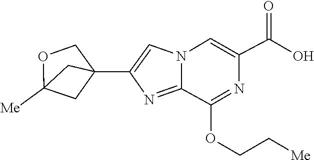 [SMILES: CCCOc1nc(C(=O)O)cn2cc(C34COC(C)(C3)C4)nc12] |
| | 2-(1-methyl-2-oxabicyclo[2.1.1]hexan-4-yl)-8-propoxyimidazo[1,2-a]pyrazine-6- |
| | carboxylic acid SM: phenyl 2-(1-methyl-2-oxabicyclo[2.1.1]hexan-4-yl)-8- |
| | propoxyimidazo[1,2-a]pyrazine-6-carboxylate (Preparation 276) LCMS m/z = |
| | 318.1 [M + H]⁺ |
| 338^{A} | 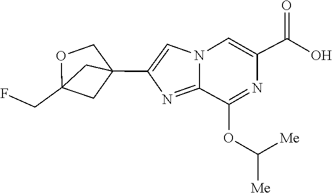 [SMILES: CC(C)Oc1nc(C(=O)O)cn2cc(C34COC(CF)(C3)C4)nc12] |
| | 2-(1-(fluoromethyl)-2-oxabicyclo[2.1.1]hexan-4-yl)-8-isopropoxyimidazo[1,2- |
| | a]pyrazine-6-carboxylic acid SM: phenyl 2-(1-(fluoromethyl)-2- |
| | oxabicyclo[2.1.1]hexan-4-yl)-8-isopropoxyimidazo[1,2-a]pyrazine-6-carboxylate |
| | (Preparation 279) LCMS m/z = 336.1 [M + H]⁺ |
| 339^{A} | 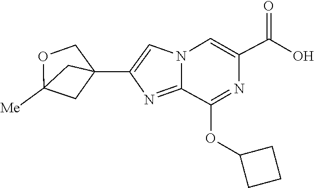 [SMILES: CC12CC(c3cn4cc(C(=O)O)nc(OC5CCC5)c4n3)(CO1)C2] |
| | 8-cyclobutoxy-2-(1-methyl-2-oxabicyclo[2.1.1]hexan-4-yl)imidazo[1,2- |
| | a]pyrazine-6-carboxylic acid SM: phenyl 8-cyclobutoxy-2-(1-methyl-2- |
| | oxabicyclo[2.1.1]hexan-4-yl)imidazo[1,2-a]pyrazine-6-carboxylate (Preparation |
| | 278) LCMS m/z = 330.1 [M + H]⁺ |
| 340^{A} | 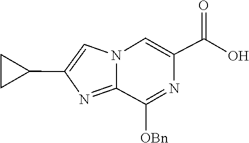 [SMILES: O=C(O)c1cn2cc(C3CC3)nc2c(OCc2ccccc2)n1] |
| | 8-(benzyloxy)-2-cyclopropylimidazo[1,2-a]pyrazine-6-carboxylic acid |
| | SM: methyl 8-(benzyloxy)-2-cyclopropylimidazo[1,2-a]pyrazine-6-carboxylate |
| | (Preparation 260) LCMS m/z = 310.1 [M + H]⁺ |

^{A}Aqueous solution extracted with EtOAc, combined organic extracts dried over MgSO₄, filtered and evaporated under reduced pressure to afford the title compound
^{B}crude product was purified by SCX ion exchange chromatography
^{C}crude product was purified by reverse phase HPLC eluting with MeCN in water at an appropriate gradient.

### Preparation 341: 2-Cyclopropyl-8-ethoxyimidazo[1,2-a]pyrazine-6-carboxylic acid

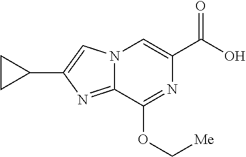 [SMILES: CCOc1nc(C(=O)O)cn2cc(C3CC3)nc12]

To a solution of methyl 2-cyclopropyl-8-ethoxyimidazo[1,2-a]pyrazine-6-carboxylate (Preparation 259, 381 mg, 1.46 mmol) in MeOH (2 mL), THF (2 mL) and H₂O (2 mL) was added LiOH.H₂O (306.3 mg, 7.30 mmol) and the reaction stirred at 22° C. for 16 h. The mixture was neutralized using 1M HCl then concentrated in vacuo to give an aqueous layer. This was extracted with EtOAc (20 mL×3), the combined organic layer was dried over MgSO₄, filtered and the filtrate evaporated under reduced pressure to afford 2-cyclopropyl-8-ethoxyimidazo[1,2-a]pyrazine-6-carboxylic acid (353 mg, 97.9% yield) as an off white solid. LCMS m/z=248.1 [M+H]⁺

### Preparation 342: Lithium 8-fluoro-7-isopropoxy-2-(tetrahydro-2H-pyran-4-yl)imidazo[1,2-a]pyridine-6-carboxylate

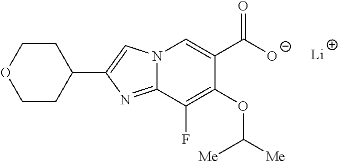 [SMILES: CC(C)Oc1c(C(=O)[O-])cn2cc(C3CCOCC3)nc2c1F ; [Li+]]

A mixture of methyl 8-fluoro-7-isopropoxy-2-(tetrahydro-2H-pyran-4-yl)imidazo[1,2-a]pyridine-6-carboxylate (Preparation 253, 60 mg, 178.38 μmol) and LiOH.H₂O (12.8 mg, 0.535 mmol) in MeOH (595 H₂O (595 μL) and THF (595 μL) was stirred overnight. The solution was evaporated under reduced pressure to afford lithium 8-fluoro-7-isopropoxy-2-(tetrahydro-2H-pyran-4-yl)imidazo[1,2-a]pyridine-6-carboxylate. LCMS m/z=323.0 [M-Li+H]⁺

### Preparation 343: 7-Cyclobutoxy-2-(1-methyl-2-oxabicyclo[2.1.1]hexan-4-yl)imidazo[1,2-a]pyridine-6-carboxylic acid

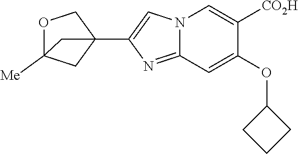 [SMILES: CC12CC(c3cn4cc(C(=O)O)c(OC5CCC5)cc4n3)(CO1)C2]

A mixture methyl 7-cyclobutoxy-2-(1-methyl-2-oxabicyclo[2.1.1]hexan-4-yl)imidazo[1,2-a]pyridine-6-carboxylate (Preparation 262, 4.50 g, 13.14 mmol) and LiOH.H₂O (606 mg, 14.45 mmol) in THF (90 mL) and H₂O (10 mL) was stirred at rt for 14 h. The THF was removed by evaporation and H₂O (50 mL) and activated carbon (1 g) were added and the mixture immediately filtered. The filtrate was acidified to pH 5-6 with c.HCl and precipitate collected by filtration, washed with water and air-dried. The residue was crystallized from MeCN (50 mL) to give 7-cyclobutoxy-2-(1-methyl-2-oxabicyclo[2.1.1]hexan-4-yl)imidazo[1,2-a]pyridine-6-carboxylic acid (3.20 g, 63%). LCMS m/z=329.2 [M+H]⁺

### Preparation 344: 8-Cyclopropoxy-2-cyclopropylimidazo[1,2-a]pyrazine-6-carboxylic acid

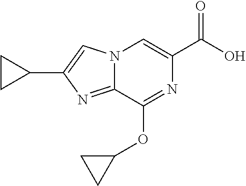 [SMILES: O=C(O)c1cn2cc(C3CC3)nc2c(OC2CC2)n1]

To a solution of methyl 8-bromo-2-cyclopropylimidazo[1,2-a]pyrazine-6-carboxylate (Preparation 242, 437.0 mg, 1.48 mmol) and cyclopropanol (784.0 mg, 13.50 mmol) in H₂O (2 mL) and THF (5 mL) was added LiOH.H₂O (186.3 mg, 4.44 mmol) and the reaction stirred at 22° C. for 16 h. The mixture was neutralized using 1M HCl (1M) and concentrated in vacuo to give an aqueous layer. This was extracted with EtOAc (20 mL×3), the combined organic layer washed with brine (30 mL), dried over MgSO₄ and filtered. The filtrate was concentrated in vacuo and the crude was purified by column chromatography (0-100% 3:1 EtOAc: EtOH in heptanes) to afford 8-cyclopropoxy-2-cyclopropylimidazo[1,2-a]pyrazine-6-carboxylic acid (74 mg, 19.3% yield) as a light yellow solid. LCMS m/z=260.0 [M+H]⁺

### Preparation 345: 7-isopropoxy-2-(1-methyl-2-oxabicyclo[2.2.1]heptan-4-yl)imidazo[1,2-a]pyrimidine-6-carboxylic acid

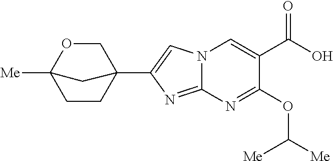 [SMILES: CC(C)Oc1nc2nc(C34CCC(C)(C3)OC4)cn2cc1C(=O)O]

Part A: A mixture of 2-bromo-1-(1-methyl-2-oxabicyclo[2.2.1]heptan-4-yl)ethan-1-one (Preparation 171, 559.4 mg, 2.40 mmol), isopropyl 2-amino-4-isopropoxypyrimidine-5-carboxylate (Preparation 206, 478.5 mg, 2.0 mmol) and NaHCO₃ (504.1 mg, 6.0 mmol) in MeCN (6.0 mL) and toluene (4.0 mL) was heated at 90° C. overnight. The cooled mixture was partitioned between EtOAc and brine, the layers separated and the aqueous layer was extracted with EtOAc. The combined organic phases were dried, filtered and concentrated in vacuo. The crude was purified by silica gel column chromatography eluting with EtOAc/heptanes (50/50 to 100/0) to give isopropyl 7-isopropoxy-2-(1-methyl-2-oxabicyclo[2.2.1]heptan-4-yl)imidazo[1,2-a]pyrimidine-6-carboxylate, as pale yellow oil. ¹H NMR (400 MHz, MeOH-d₄) δ: 1.39 (d, 6H), 1.45 (d, 6H), 1.47 (s, 3H), 1.76-2.21 (m, 6H), 3.91 (d, 1H), 4.04 (dd, 1H), 5.23 (td, 1H), 5.52 (quin, 1H), 7.48 (s, 1H), 9.15 (s, 1H)

Part B: A solution of isopropyl 7-isopropoxy-2-(1-methyl-2-oxabicyclo[2.2.1]heptan-4-yl)imidazo[1,2-a]pyrimidine-6-carboxylate (275 mg, 0.736 mmol) in 1M NaOH (736 THF (2.0 mL) and MeOH (2.0 mL) was stirred at rt for 2 h. The mixture was acidified to pH 3 using 1N HCl, the solution evaporated under reduced pressure and the solid lyophilised to provide 7-isopropoxy-2-(1-methyl-2-oxabicyclo[2.2.1]heptan-4-yl)imidazo[1,2-a]pyrimidine-6-carboxylic acid, as a white powder. LCMS m/z=332.2 [M+H]⁺

The following carboxylic acids were prepared by analogy with the procedure described for Preparation 298, or as described in Scheme II, via compounds of Formulae (V) and (VIII), wherein PG is Me.

| Prep. | |
|---|---|
| No | Structure/Name |
| 346 | 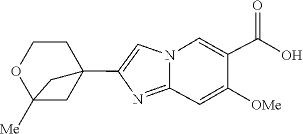 [SMILES: COc1cc2nc(C34CCOC(C)(C3)C4)cn2cc1C(=O)O] |
| | 7-methoxy-2-(1-methyl-2-oxabicyclo[3.1.1]heptan-5-yl)imidazo[1,2- |
| | a]pyridine-6-carboxylic acid LCMS m/z = 303.1 [M + H]⁺ |
| 347 | 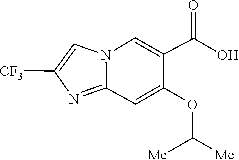 [SMILES: CC(C)Oc1cc2nc(C(F)(F)F)cn2cc1C(=O)O] |
| | 7-isopropoxy-2-(trifluoromethyl)imidazo[1,2-a]pyridine-6- |
| | carboxylic acid LCMS m/z = 289.0 [M + H]⁺ |
| 348 | 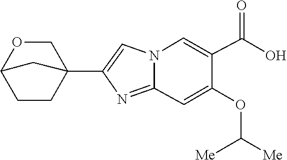 [SMILES: CC(C)Oc1cc2nc(C34CCC(C3)OC4)cn2cc1C(=O)O] |
| | 2-(2-oxabicyclo[2.2.1]heptan-4-yl)-7-isopropoxyimidazo[1,2-a] |
| | pyridine-6-carboxylic acid LCMS m/z = 317.1 [M + H]⁺ |
| 349 | 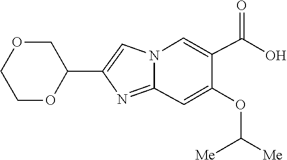 [SMILES: CC(C)Oc1cc2nc(C3COCCO3)cn2cc1C(=O)O] |
| | 2-(1,4-dioxan-2-yl)-7-isopropoxyimidazo[1,2-a]pyridine-6- |
| | carboxylic acid LCMS m/z = 307.1 [M + H]⁺ |
| 350 | 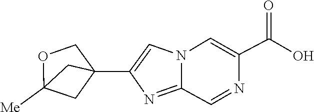 [SMILES: CC12CC(c3cn4cc(C(=O)O)ncc4n3)(CO1)C2] |
| | 2-(1-methyl-2-oxabicyclo[2.1.1]hexan-4-yl)imidazo[1,2-a]pyrazine- |
| | 6-carboxylic acid LCMS m/z = 260.0 [M + H]⁺ |
| 351 | 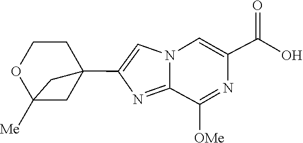 [SMILES: COc1nc(C(=O)O)cn2cc(C34CCOC(C)(C3)C4)nc12] |
| | 8-methoxy-2-(1-methyl-2-oxabicyclo[3.1.1]heptan-5-yl)imidazo[1,2- |
| | a]pyrazine-6-carboxylic acid LCMS m/z = 304.1 [M + H]⁺ |
| 352 | 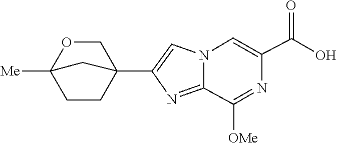 [SMILES: COc1nc(C(=O)O)cn2cc(C34CCC(C)(C3)OC4)nc12] |
| | 8-methoxy-2-(1-methyl-2-oxabicyclo[2.2.1]heptan-4-yl)imidazo |
| | [1,2-a]pyrazine-6-carboxylic acid |
| | ¹H NMR (400 MHz, CDCl₃) δ: 1.48 (s, 3H), 1.78-2.25 (m, 6H), |
| | 3.91-4.16 (m, 2H), 4.24 (s, 3H), 7.54 (br d, 1H), 8.62 (br s, 1H) |
| 353 | 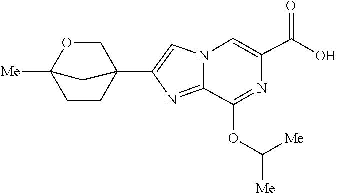 [SMILES: CC(C)Oc1nc(C(=O)O)cn2cc(C34CCC(C)(C3)OC4)nc12] |
| | 8-isopropoxy-2-(1-methyl-2-oxabicyclo[2.2.1]heptan-4-yl)imidazo |
| | [1,2-a]pyrazine-6-carboxylic acid LCMS m/z = 332.2 [M + H]⁺ |
| 354 | 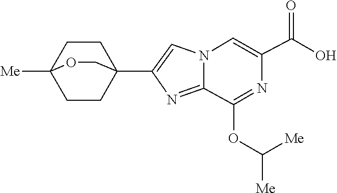 [SMILES: CC(C)Oc1nc(C(=O)O)cn2cc(C34CCC(C)(CC3)OC4)nc12] |
| | 8-isopropoxy-2-(1-methyl-2-oxabicyclo[2.2.2]octan-4-yl)imidazo |
| | [1,2-a]pyrazine-6-carboxylic acid LCMS m/z = 346.2 [M + H]⁺ |
| 355 | 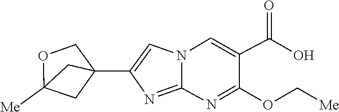 [SMILES: CCOc1nc2nc(C34COC(C)(C3)C4)cn2cc1C(=O)O] |
| | 7-ethoxy-2-(1-methyl-2-oxabicyclo[2.1.1]hexan-4-yl)imidazo |
| | [1,2-a]pyrimidine-6-carboxylic acid LCMS m/z = 304.1 [M + H]⁺ |
| 356 | 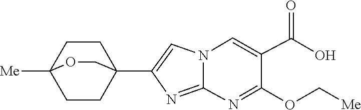 [SMILES: CCOc1nc2nc(C34CCC(C)(CC3)OC4)cn2cc1C(=O)O] |
| | 7-ethoxy-2-(1-methyl-2-oxabicyclo[2.2.2]octan-4-yl)imidazo[1,2- |
| | a]pyrimidine-6-carboxylic acid LCMS m/z = 332.1 [M + H]⁺ |

The following carboxylic acids were prepared by analogy with the procedure described for Preparation 312, or as described in Scheme II, via compounds of formulae (V) and (VIII), wherein PG is isopropyl.

| Prep. No | Structure/Name/Data |
|---|---|
| 357 | 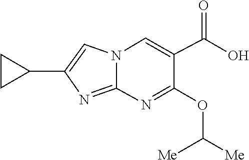 [SMILES: CC(C)Oc1nc2nc(C3CC3)cn2cc1C(=O)O] |
| | 2-cyclopropyl-7-isopropoxyimidazo[1,2-a]pyrimidine-6- |
| | carboxylic acid LCMS m/z = 262.1 [M + H]⁺ |
| 358 | 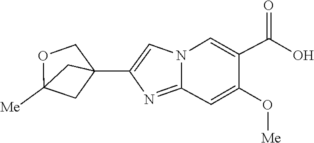 [SMILES: COc1cc2nc(C34COC(C)(C3)C4)cn2cc1C(=O)O] |
| | 7-methoxy-2-(1-methyl-2-oxabicyclo[2.1.1]hexan-4-yl)imidazo |
| | [1,2-a]pyridine-6-carboxylic acid LCMS m/z = 289.1 [M + H]⁺ |
| 359 | 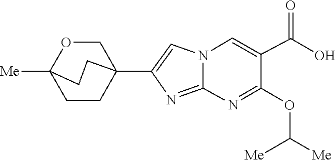 [SMILES: CC(C)Oc1nc2nc(C34CCC(C)(CC3)OC4)cn2cc1C(=O)O] |
| | 7-isopropoxy-2-(1-methyl-2-oxabicyclo[2.2.2]octan-4-yl)imidazo |
| | [1,2-a]pyrimidine-6-carboxylic acid LCMS m/z = 346.1 [M + H]⁺ |

The following carboxylic acids were prepared by analogy with the procedure described for Preparation 313, or as described in Scheme II, via compounds of Formulae (IV), (VII) and (VIII), wherein PG is methyl.

| Prep. No | Structure/Name/Data |
|---|---|
| 360 | 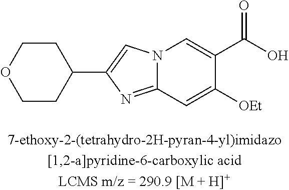 [SMILES: CCOc1cc2nc(C3CCOCC3)cn2cc1C(=O)O] |
| 361 | 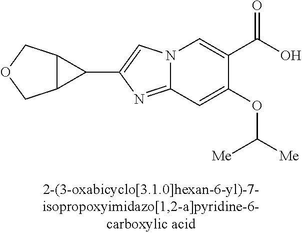 [SMILES: CC(C)Oc1cc2nc(C3C4COCC43)cn2cc1C(=O)O] |
| 362 | 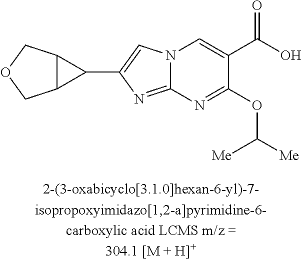 [SMILES: CC(C)Oc1nc2nc(C3C4COCC43)cn2cc1C(=O)O] |

The following carboxylic acids were prepared by analogy with the procedure described for Preparation 310, or as described in Scheme II, via compounds of Formulae (IV), (VII) and (VIII), wherein PG is phenyl.

| Prep. No | Structure/Name/Data |
|---|---|
| 363 | 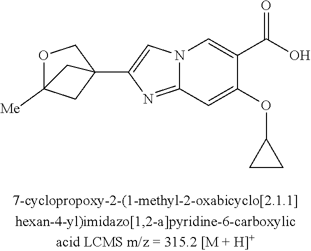 [SMILES: CC12CC(c3cn4cc(C(=O)O)c(OC5CC5)cc4n3)(CO1)C2] |
| 364 | 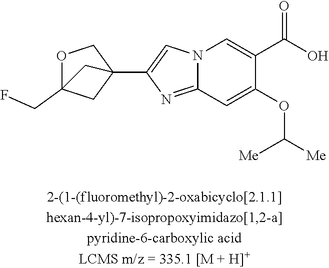 [SMILES: CC(C)Oc1cc2nc(C34COC(CF)(C3)C4)cn2cc1C(=O)O] |
| 365 | 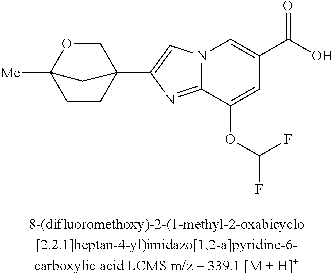 [SMILES: CC12CCC(c3cn4cc(C(=O)O)cc(OC(F)F)c4n3)(CO1)C2] |
| 366 | 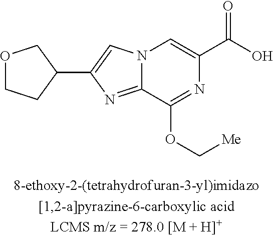 [SMILES: CCOc1nc(C(=O)O)cn2cc(C3CCOC3)nc12] |
| 367 | 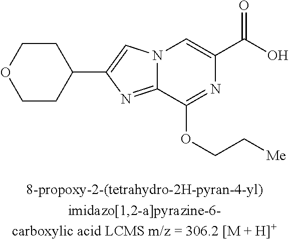 [SMILES: CCCOc1nc(C(=O)O)cn2cc(C3CCOCC3)nc12] |
| 368 | 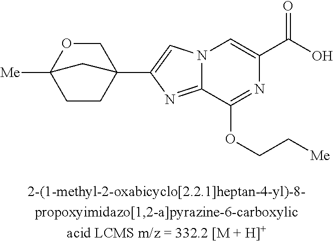 [SMILES: CCCOc1nc(C(=O)O)cn2cc(C34CCC(C)(C3)OC4)nc12] |
| 369 | 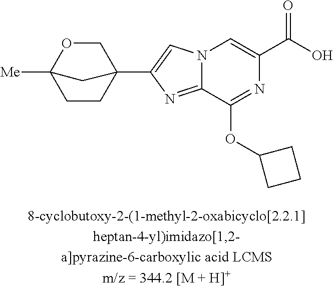 [SMILES: CC12CCC(c3cn4cc(C(=O)O)nc(OC5CCC5)c4n3)(CO1)C2] |
| 370 | 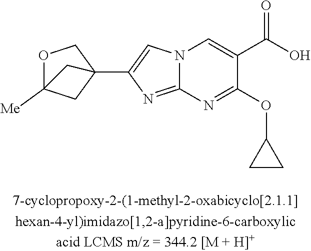 [SMILES: CC12CC(c3cn4cc(C(=O)O)c(OC5CC5)nc4n3)(CO1)C2] |
| 371 | 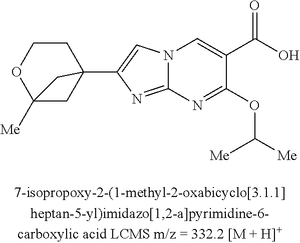 [SMILES: CC(C)Oc1nc2nc(C34CCOC(C)(C3)C4)cn2cc1C(=O)O] |

### Preparation 372: 2-Amino-4-isopropoxypyrimidine-5-carboxylic acid

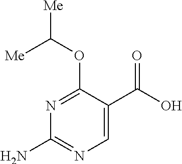 [SMILES: CC(C)Oc1nc(N)ncc1C(=O)O]

To a solution of isopropyl 2-amino-4-isopropoxypyrimidine-5-carboxylate (Preparation 206, 239 mg, 1.0 mmol) in THF (2 mL) and MeOH (2 mL) was added NaOH (1 M, 2 mL) and the mixture stirred at rt for 2 days. The reaction was acidified to pH 3˜4 by the addition of 1N HCl, evaporated to dryness and lyophilized to afford 2-amino-4-isopropoxypyrimidine-5-carboxylic acid as a pale brown powder (314 mg, 100%). ¹H NMR (400 MHz, MeOH-d₄) δ: 1.39 (d, 6H), 5.51 (quin, 1H), 8.60 (s, 1H).

### Preparation 373: 2-Amino-4-isopropoxy-N-(pyrazolo[1,5-a]pyrimidin-3-yl)pyrimidine-5-carboxamide

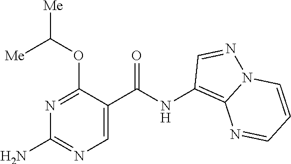 [SMILES: CC(C)Oc1nc(N)ncc1C(=O)Nc1cnn2cccnc12]

HATU (100 mg, 0.263 mmol) and DIPEA (129 mg, 1.0 mmol) were added to 2-amino-4-isopropoxypyrimidine-5-carboxylic acid (Preparation 372, 78.5 mg, 0.250 mmol) and pyrazolo[1,5-a]pyrimidin-3-amine (35.2 mg, 0.263 mmol) in DMF (1.5 mL) and the mixture stirred at rt overnight. The reaction was evaporated to dryness and the residue triturated with MeCN/EtOAc/water. The solid was collected by filtration and washed with water, MeCN and EtOAc to afford 2-amino-4-isopropoxy-N-(pyrazolo[1,5-a]pyrimidin-3-yl)pyrimidine-5-carboxamide as a yellow solid (58 mg, 74%). LCMS m/z=314.1 [M+H]⁺.

### Preparation 374: 2-Amino-4-isopropoxy-N-(6-methylpyrazolo[1,5-a]pyrimidin-3-yl)pyrimidine-5-carboxamide

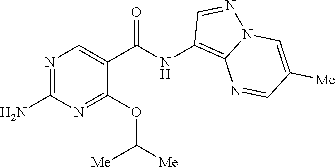 [SMILES: Cc1cnc2c(NC(=O)c3cnc(N)nc3OC(C)C)cnn2c1]

was obtained as a brown solid, 173 mg, 70.5% yield, from 2-amino-4-isopropoxypyrimidine-5-carboxylic acid (Preparation 372) and 6-methylpyrazolo[1,5-a]pyrimidin-3-amine following a similar procedure to that described in Preparation 373. LCMS m/z=328.1 [M+H]⁺

### Preparation 375: 7-(Benzyloxy)-N-(1-methyl-1H-pyrazol-3-yl)-2-(1-methyl-2-oxabicyclo[2.1.1]hexan-4-yl)imidazo[1,2-a]pyridine-6-carboxamide

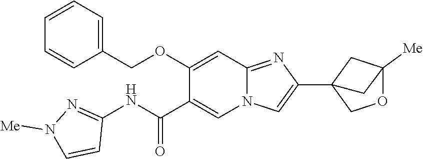 [SMILES: Cn1ccc(NC(=O)c2cn3cc(C45COC(C)(C4)C5)nc3cc2OCc2ccccc2)n1]

To a solution of 7-(benzyloxy)-2-(1-methyl-2-oxabicyclo[2.1.1]hexan-4-yl)imidazo[1,2-a]pyridine-6-carboxylic acid (Preparation 308, 3.0 g, 8.23 mmol) in DMF (20 mL) was added HATU (3.77 g, 9.88 mmol), DIPEA (3.19 g, 24.69 mmol) and 1-methylpyrazol-3-amine (879.2 mg, 9.05 mmol) and the mixture stirred at rt for 48 h. The reaction was diluted with H₂O (100 mL) and extracted with EtOAc (2×50 mL). The combined organics were washed with H₂O (50 mL), brine (50 mL), dried (Na₂SO₄) and evaporated to dryness in vacuo to afford a brown solid. The solid was crystallized from H₂O (50 mL) and the precipitate collected by filtration, washed with H₂O and air-dried to give 7-(benzyloxy)-N-(1-methyl-1H-pyrazol-3-yl)-2-(1-methyl-2-oxabicyclo[2.1.1]hexan-4-yl)imidazo[1,2-a]pyridine-6-carboxamideas a white solid (2.90 g, 77.0%). LCMS m/z=444.2 [M+H]⁺

### Preparation 376: 7-(Benzyl oxy)-N-(1-(difluoromethyl)-2-oxo-1,2-dihydropyridin-3-yl)-2-(1-methyl-2-oxabicyclo[2.1.1]hexan-4-yl)imidazo[1,2-a]pyridine-6-carboxamide

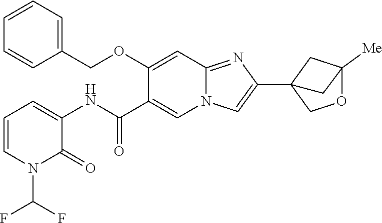 [SMILES: CC12CC(c3cn4cc(C(=O)Nc5cccn(C(F)F)c5=O)c(OCc5ccccc5)cc4n3)(CO1)C2]

was prepared as a white solid (3.0 g, 72%) from 7-(benzyloxy)-2-(1-methyl-2-oxabicyclo[2.1.1]hexan-4-yl)imidazo[1,2-a]pyridine-6-carboxylic acid (Preparation 308) and 3-amino-1-(difluoromethyl)pyridin-2-one using an analogous method to that described for Preparation 375. LCMS m/z=507.2 [M+H]⁺

Preparation 377: 7-(Benzyloxy)-2-cyclopropyl-N-(1-(difluoromethyl)-2-oxo-1,2-dihydropyridin-3-yl)imidazo[1,2-a]pyridine-6-carboxamide

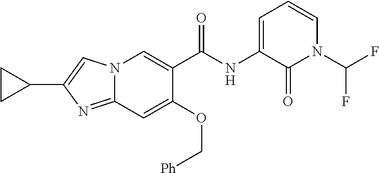 [SMILES: O=C(Nc1cccn(C(F)F)c1=O)c1cn2cc(C3CC3)nc2cc1OCc1ccccc1]

was obtained as a white solid, 2.65 g, 60% yield, from 3-amino-1-(difluoromethyl)pyridin-2-one and 7-(benzyloxy)-2-cyclopropylimidazo[1,2-a]pyridine-6-carboxylic acid (Preparation 300) following the procedure described in Preparation 375. LCMS m/z=451.2 [M+H]⁺.

### Preparation 378: 7-(Benzyloxy)-2-cyclopropyl-N-(1-methyl-1H-pyrazol-3-yl)imidazo[1,2-a]pyridine-6-carboxamide

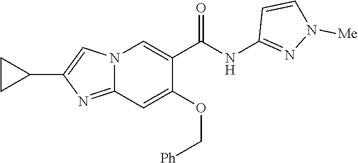 [SMILES: Cn1ccc(NC(=O)c2cn3cc(C4CC4)nc3cc2OCc2ccccc2)n1]

To a solution of 7-(benzyloxy)-2-cyclopropylimidazo[1,2-a]pyridine-6-carboxylic acid (Preparation 300, 3.0 g, 9.73 mmol) in dioxane (100 mL) was added CDI (1.89 g, 11.68 mmol) and TEA (1.08 g, 10.7 mmol) and the resulting reaction mixture stirred for 4 h at 90° C. 1-Methylpyrazol-3-amine (1.04 g, 10.7 mmol) was added and the mixture stirred at 100° C. for 72 h. The reaction mixture was evaporated to dryness in vacuo and the residue treated with H₂O (50 mL) with cooling. The resulting precipitate was collected by filtration and washed with hexane to afford 7-(benzyloxy)-2-cyclopropyl-N-(1-methyl-1H-pyrazol-3-yl)imidazo[1,2-a]pyridine-6-carboxamide (2.30 g, 56%). LCMS m/z=388.0 [M+H]⁺.

### Preparation 379: 7-(Benzyloxy)-2-cyclopropyl-N-(2-methoxypyridin-3-yl)imidazo[1,2-a]pyridine-6-carboxamide

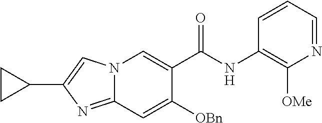 [SMILES: COc1ncccc1NC(=O)c1cn2cc(C3CC3)nc2cc1OCc1ccccc1]

Methanesulfonyl chloride (40.8 mg, 0.357 mmol) was added dropwise to a solution 7-(benzyloxy)-2-cyclopropylimidazo[1,2-a]pyridine-6-carboxylic acid (Preparation 300, 100 mg, 0.324 mmol) and TEA (37.7 mg, 0.373 mmol) in MeCN (5 mL) at −15° C. The reaction mixture was warmed to 0° C. for 0.5 h and 2-methoxypyridin-3-amine (60.4 mg, 0.487 mmol) added and stirred at rt for 48 h. The reaction mixture was evaporated to dryness in vacuo to afford 7-(benzyloxy)-2-cyclopropyl-N-(2-methoxypyridin-3-yl)imidazo[1,2-a]pyridine-6-carboxamide as a yellow oil (80 mg, crude). LCMS m/z=415.2 [M+H]⁺

### Preparation 380: 7-(Benzyloxy)-2-cyclopropyl-N-(6-methoxypyridin-2-yl)imidazo[1,2-a]pyridine-6-carboxamide

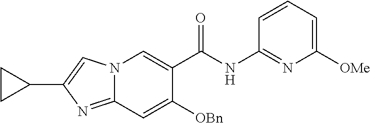 [SMILES: COc1cccc(NC(=O)c2cn3cc(C4CC4)nc3cc2OCc2ccccc2)n1]

Methanesulfonyl chloride (1.23 g, 10.70 mmol) was added dropwise to a solution 7-(benzyloxy)-2-cyclopropylimidazo[1,2-a]pyridine-6-carboxylic acid (Preparation 300, 3.0 g, 9.73 mmol) and TEA (1.13 g, 11.19 mmol) in MeCN (50 mL) at −15° C. The reaction mixture was warmed to 0° C. for 0.5 h and then 6-methoxypyridin-2-amine (2.42 g, 19.46 mmol) was added. The reaction was stirred at rt for 72 h, diluted with H₂O (50 mL) and extracted with DCM (3×50 mL). The combined organics were washed with brine, dried (Na₂SO₄) and evaporated to dryness in vacuo. The residue was crystallized from EtOH/H₂O (20 mL/60 mL) and the solid collected by filtration and washed with water (20 mL) to afford 7-(benzyloxy)-2-cyclopropyl-N-(6-methoxypyridin-2-yl)imidazo[1,2-a]pyridine-6-carboxamide as a white solid (2.10 g, 52.0% yield). LCMS m/z=415.0 [M+H]t

### Preparation 381: 7-(Benzyloxy)-2-cyclopropyl-N-(6-(difluoromethyl)pyridin-2-yl)imidazo[1,2-a]pyridine-6-carboxamide

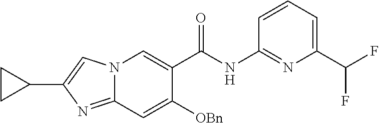 [SMILES: O=C(Nc1cccc(C(F)F)n1)c1cn2cc(C3CC3)nc2cc1OCc1ccccc1]

was prepared from 7-(benzyloxy)-2-cyclopropyl-imidazo[1,2-a]pyridine-6-carboxylic acid (Preparation 300) and 6-(difluoromethyl)pyridin-2-amine, using an analogous method to that described for Preparation 380. LCMS m/z=435.2 [M+H]⁺.

### Preparation 382: 8-(Benzyloxy)-2-cyclopropyl-N-(1-methyl-2-oxo-1,2-dihydropyridin-3-yl)imidazo[1,2-a]pyrazine-6-carboxamide

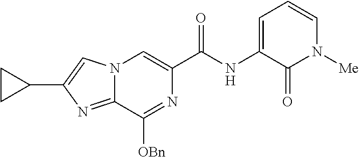 [SMILES: Cn1cccc(NC(=O)c2cn3cc(C4CC4)nc3c(OCc3ccccc3)n2)c1=O]

To a mixture of 3-amino-1-methylpyridin-2-one (516.7 mg, 4.16 mmol), 8-(benzyloxy)-2-cyclopropylimidazo[1,2-a]pyrazine-6-carboxylic acid (Preparation 340, 1.03 g, 3.33 mmol) in pyridine (11 mL) was added T3P® (6.36 g, 9.99 mmol, 50% EtOAc solution) and the reaction was capped and stirred at 22° C. overnight. The mixture was diluted with EtOAc and water and the layers separated. The aqueous phase was extracted with EtOAc (5 mL×3), the combined organic layers dried over MgSO₄, and filtered. The filtrate was evaporated in vacuo and the residue purified by Isco automatic purification system (0-50% 3:1 EtOAc: EtOH in heptanes) to afford 8-(benzyloxy)-2-cyclopropyl-N-(1-methyl-2-oxo-1,2-dihydropyridin-3-yl)imidazo[1,2-a]pyrazine-6-carboxamide (712 mg, 51.4% yield) as an off-white solid. LCMS m/z=416.2 [M+H]⁺

### Preparation 383: 7-(Benzyloxy)-6-bromo-2-(tetrahydro-2H-pyran-4-yl)imidazo[1,2-a]pyridine

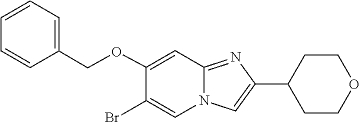 [SMILES: Brc1cn2cc(C3CCOCC3)nc2cc1OCc1ccccc1]

To a solution of 4-(benzyloxy)-5-bromopyridin-2-amine hydrobromide (Preparation 199A, 20.0 g, 55.6 mmol) and NaHCO₃ (14.5 g, 172 mmol) in EtOH (280 mL), was added 2-bromo-1-(tetrahydro-2H-pyran-4-yl)ethan-1-one (11.5 g, 55.6 mmol) and the reaction heated at reflux for 16 h. Further 2-bromo-1-(tetrahydro-2H-pyran-4-yl)ethan-1-one (5.75 g, 27.8 mmol) was added and the reaction heated at reflux for another 24 h. The cooled mixture was filtered through Celite®, the filtrate was poured over heptanes (3.5 L) and the resulting suspension stirred for 1 h at rt. The mixture was filtered through Celite® and the filtrate concentrated in vacuo. The crude product was stirred in TBME (70 mL), the solid was filtered off, washed with small amounts of TBME and dried in vacuo, to provide 7-(benzyloxy)-6-bromo-2-(tetrahydro-2H-pyran-4-yl)imidazo[1,2-a]pyridine (16.4 g, 76%) as a yellow solid.

### Preparation 384: Methyl 7-(benzyloxy)-2-(tetrahydro-2H-pyran-4-yl)imidazo[1,2-a]pyridine-6-carboxylate

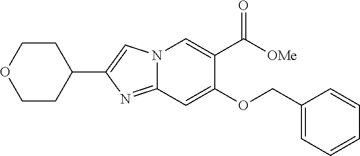 [SMILES: COC(=O)c1cn2cc(C3CCOCC3)nc2cc1OCc1ccccc1]

A mixture of 7-(benzyloxy)-6-bromo-2-(tetrahydro-2H-pyran-4-yl)imidazo[1,2-a]pyridine (Preparation 383, 11.0 g, 28.4 mmol), PPh₃ (1.07 g, 4.06 mmol), Pd(OAc)₂ (740 mg, 3.29 mmol), TEA (11.0 mL, 78.7 mmol) in MeOH (160 mL) was flushed with CO (5×10 bar) in an autoclave. The autoclave was charged with CO (10 bar) and heated at 120° C. for 3 h. The autoclave was cooled to 50° C. and recharged with CO (10.0 bar) and stirred further at 120° C. for 17 h. The cooled mixture was concentrated in vacuo, the residue was suspended in DCM (250 mL) and filtered through Celite®, washing through with DCM (2×20 mL). The filtrates were washed with sat. aq. NH₄C1 (150 mL), the aqueous layer was extracted with DCM (3×50 mL), the organic layers were combined and dried over Na₂SO₄. The residue was purified by silica gel column chromatography using an automated purification system eluting with DCM/MeOH to afford methyl 7-(benzyloxy)-2-(tetrahydro-2H-pyran-4-yl)imidazo[1,2-a]pyridine-6-carboxylate (6.24 g, 47.0%). LCMS m/z=367.2 [M+H]⁺

### Preparation 385: 7-Hydroxy-N-(6-methoxypyridin-2-yl)-2-(tetrahydro-2H-pyran-4-yl)imidazo[1,2-a]pyridine-6-carboxamide

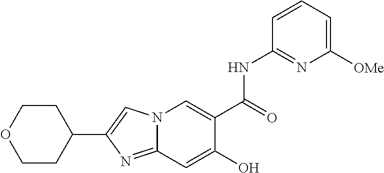 [SMILES: COc1cccc(NC(=O)c2cn3cc(C4CCOCC4)nc3cc2O)n1]

Part A: nBuLi (5.8 mL, 2.5 M in Hexanes) was added to a solution of 6-methoxypyridin-2-amine (2.03 g, 16 mmol) in THF (100 mL) at −78° C. under Ar and the resulting dark-brown solution stirred at the same temperature for 30 min. A solution of methyl 7-(benzyloxy)-2-(tetrahydro-2H-pyran-4-yl)imidazo[1,2-a]pyridine-6-carboxylate (Preparation 384, 2.40 g, 6.5 mmol) in THF (25 mL) was added via syringe and the solution stirred at rt overnight. The reaction was quenched with NH₄C1 solution (50 mL) and the organic phase separated and evaporated to dryness in in vacuo. The residue was recrystallized from EtOAc/Hex to afford 7-(benzyloxy)-N-(6-methoxypyridin-2-yl)-2-(tetrahydro-2H-pyran-4-yl)imidazo[1,2-a]pyridine-6-carboxamide (2.2 g, 73%).

Part B: Pd/C (5% on carbon, 72 mg) was added to a solution of 7-(benzyloxy)-N-(6-methoxypyridin-2-yl)-2-(tetrahydro-2H-pyran-4-yl)imidazo[1,2-a]pyridine-6-carboxamide (2.2 g, 4.8 mmol) in MeOH (100 mL). The reaction mixture was deoxygenated and saturated with H2 and then stirred at rt for 4 h. The reaction mixture was evaporated to dryness in vacuo and the residue taken up in hot DMF (100 mL). The catalyst was removed by filtration and the filtrate evaporated to dryness in vacuo. The residue was washed with MeOH (50 mL), water (150 mL) and dried to afford 7-hydroxy-N-(6-methoxypyridin-2-yl)-2-(tetrahydro-2H-pyran-4-yl)imidazo[1,2-a]pyridine-6-carboxamide (0.56 g, 31.6%). LCMS m/z=369.0 [M+H]⁺

### Preparation 386: N-(6-(difluoromethyl)pyridin-2-yl)-7-hydroxy-2-(tetrahydro-2H-pyran-4-yl)imidazo[1,2-a]pyridine-6-carboxamide

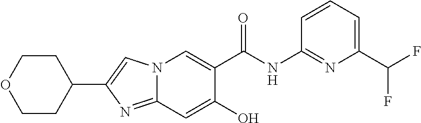 [SMILES: O=C(Nc1cccc(C(F)F)n1)c1cn2cc(C3CCOCC3)nc2cc1O]

was prepared from methyl 7-(benzyloxy)-2-(tetrahydro-2H-pyran-4-yl)imidazo[1,2-a]pyridine-6-carboxylate (Preparation 384) and 6-(difluoromethyl)pyridin-2-amine using an analogous 2-step method to that described for Preparation 385. LCMS m/z=389.2 [M+H]⁺.

### Preparation 387: 7-Hydroxy-N-(1-methyl-1H-pyrazol-3-yl)-2-(1-methyl-2-oxabicyclo[2.1.1]hexan-4-yl)imidazo[1,2-a]pyridine-6-carboxamide

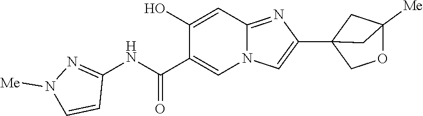 [SMILES: Cn1ccc(NC(=O)c2cn3cc(C45COC(C)(C4)C5)nc3cc2O)n1]

To a solution of 7-(benzyloxy)-N-(1-methyl-1H-pyrazol-3-yl)-2-(1-methyl-2-oxabicyclo[2.1.1]hexan-4-yl)imidazo[1,2-a]pyridine-6-carboxamide (Preparation 375, 2.90 g, 6.54 mmol) in MeOH (300 mL) was added Pd/C (348 mg, 10% purity) and the reaction mixture was stirred at rt under an atmosphere of H2 for 6 h. The solids were removed by filtration and the filtrate evaporated to dryness in vacuo to afford 7-hydroxy-N-(1-methyl-1H-pyrazol-3-yl)-2-(1-methyl-2-oxabicyclo[2.1.1]hexan-4-yl)imidazo[1,2-a]pyridine-6-carboxamide as a yellow solid (2.25 g, 97%). LCMS m/z=354.2 [M+H]⁺

### Preparations 388 to 393

The following compounds were prepared from the appropriate benzyl ether, following a similar procedure to that described in Preparation 384.

| Prep. No | Name/Structure/Starting Material (SM)/Yield/Data |
|---|---|
| 388 | 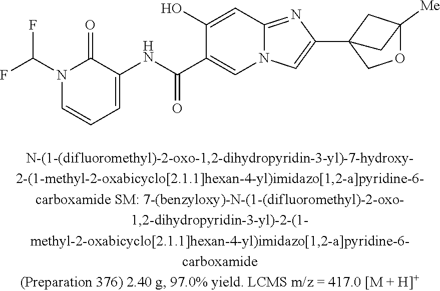 [SMILES: CC12CC(c3cn4cc(C(=O)Nc5cccn(C(F)F)c5=O)c(O)cc4n3)(CO1)C2] |
| 389 | 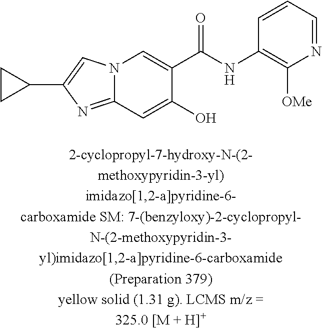 [SMILES: COc1ncccc1NC(=O)c1cn2cc(C3CC3)nc2cc1O] |
| 390 | 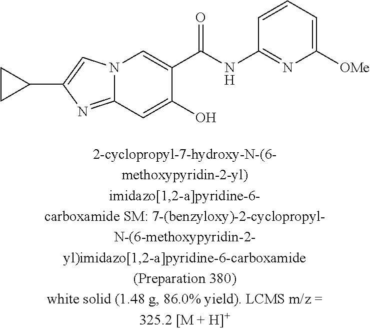 [SMILES: COc1cccc(NC(=O)c2cn3cc(C4CC4)nc3cc2O)n1] |
| 391 | 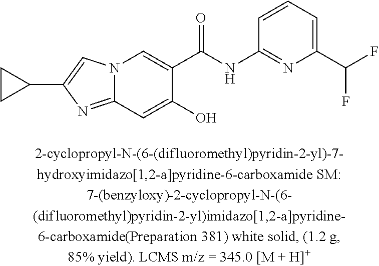 [SMILES: O=C(Nc1cccc(C(F)F)n1)c1cn2cc(C3CC3)nc2cc1O] |
| 392 | 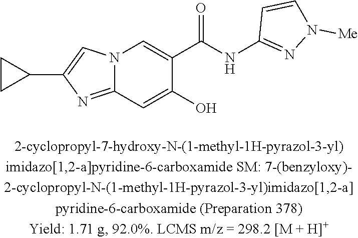 [SMILES: Cn1ccc(NC(=O)c2cn3cc(C4CC4)nc3cc2O)n1] |
| 393 | 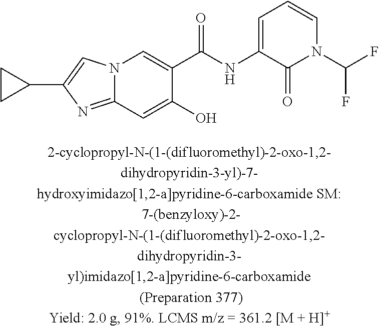 [SMILES: O=C(Nc1cccn(C(F)F)c1=O)c1cn2cc(C3CC3)nc2cc1O] |

### Preparation 394: 2-Cyclopropyl-8-hydroxy-N-(1-methyl-2-oxo-1,2-dihydropyridin-3-yl)imidazo[1,2-a]pyrazine-6-carboxamide

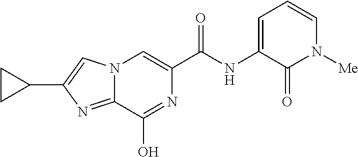 [SMILES: Cn1cccc(NC(=O)c2cn3cc(C4CC4)nc3c(O)n2)c1=O]

A solution of 8-(benzyloxy)-2-cyclopropyl-N-(1-methyl-2-oxo-1,2-dihydropyridin-3-yl)imidazo[1,2-a]pyrazine-6-carboxamide (Preparation 382, 712 mg, 1.71 mmol) in THF (8 mL) and MeOH (8 mL) in a pressure vessel charged with Pd/C (182.0 mg, 0.171 mmol) was stirred at rt under 15 psi of H2 for 3 h. The mixture was filtered, dried, re-dissolved in MeOH/THF, further Pd/C added and the reaction stirred under 30 psi of H2 overnight. The mixture was filtered washing through with MeOH. The filtrate was evaporated under reduced pressure to afford 2-cyclopropyl-8-hydroxy-N-(1-methyl-2-oxo-1,2-dihydropyridin-3-yl)imidazo[1,2-a]pyrazine-6-carboxamide (486.8 mg, 87.5% yield) as an off-white solid. LCMS m/z=326.1 [M+H]⁺

### Preparation 395: rac-(R)-4-(sec-butoxy)pyrimidin-2-amine

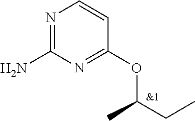

To a solution of rac-(R)-butan-2-ol (6.87 g, 92.64 mmol, 8.48 mL, 4.0 eq.) in THF (100.00 mL) was added sodium hydride (3.71 g, 92.64 mmol, 60% purity, 4.0 eq.) at 0° C. under N₂. The mixture was stirred at 0° C. for 30 min. To the reaction mixture was added 4-chloropyrimidin-2-amine (3.00 g, 23.16 mmol, 1.0 eq.). The mixture was stirred at 60° C. for 14 hours. The reaction was quenched with water (40 mL). THF was evaporated under vacuum to give the residue. The residue was diluted with water (80 mL), extracted with EtOAc (70 mL×3). The combined organic layer was washed with brine (60 mL×2), dried over Na₂SO₄; filtered and evaporated under vacuum. The residue was purified by Combi-Flash (PE: EA from 3:1 to 0:1) to give rac-(R)-4-(sec-butoxy)pyrimidin-2-amine (2.90 g, 67.40% yield) as a white solid. LCMS: m/z=168.3 [M+H]⁺. ¹H NMR: (400 MHz, CDCl₃) δ: 0.94 (t, J=7.6 Hz, 3H), 1.29 (d, J=6.0 Hz, 3H), 1.65-1.58 (m, 1H), 1.76-1.66 (m, 1H), 4.88 (brs, 2H), 5.12-5.07 (m, 1H), 6.04 (d, J=6.0 Hz, 1H), 7.99 (d, J=5.6 Hz, 1H).

### Preparation 396: rac-(R)-4-(sec-butoxy)-5-iodopyrimidin-2-amine

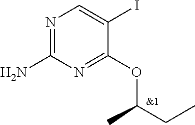

To a solution of rac-(R)-4-(sec-butoxy)pyrimidin-2-amine (2.90 g, 17.34 mmol, 1.0 eq.) in DCM (80.00 mL) was added NIS (4.71 g, 20.93 mmol, 1.0 eq.) at 0° C. The mixture was stirred at 20° C. for 14 hours. LCMS showed 48.5% of the desired product was obtained and 50.0% of the starting material remained. To the reaction was added NIS (1.95 g, 8.67 mmol, 0.5 eq.) at 0° C. The reaction was stirred at 20° C. for another 5 hours. The reaction was quenched with saturate aq.Na₂SO₃ (30 mL) and it was extracted with EtOAc (40 mL×2), dried over Na₂SO₄, filtered; evaporated under vacuum. The residue was purified by Combi-Flash (PE: EA from 3:1 to 0:1) to give rac-(R)-4-(sec-butoxy)-5-iodopyrimidin-2-amine (3.00 g, 53.11% yield) as a yellow solid. LCMS: m/z=294.2 [M+H]t ¹H NMR: (500 MHz, CDCl₃) δ: 0.97 (t, J=7.5 Hz, 3H), 1.32 (d, J=6.5 Hz, 3H), 1.71-1.63 (m, 1H), 1.78-1.71 (m, 1H), 4.91 (brs, 2H), 5.15-5.10 (m, 1H), 8.24 (s, 1H).

### Preparation 397: rac-(R)-7-(sec-butoxy)-6-iodo-2-(1-methyl-2-oxabicyclo[2.1.1]hexan-4-yl)imidazo[1,2-a]pyrimidine

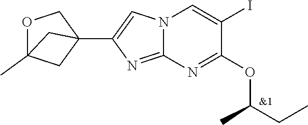 [SMILES: CC[C@@H](C)Oc1nc2nc(C34COC(C)(C3)C4)cn2cc1I]

To a solution of rac-(R)-4-(sec-butoxy)-5-iodopyrimidin-2-amine (300.0 mg, 1.02 mmol, 1.0 eq.) in tert-Butanol (10.00 mL) was added NaHCO₃ (171.4 mg, 2.04 mmol, 2.0 eq.) and 2-bromo-1-(1-methyl-2-oxabicyclo[2.1.1]hexan-4-yl)ethan-1-one (300 mg, 1.37 mmol, 1.34 eq.) at 20° C. The reaction was stirred at 90° C. for 14 hours. The reaction was evaporated under vacuum. The residue was purified by Combi-Flash (PE: EA from 3:1 to 1:1) to give rac-(R)-7-(sec-butoxy)-6-iodo-2-(1-methyl-2-oxabicyclo[2.1.1]hexan-4-yl)imidazo[1,2-a]pyrimidine (270 mg, 57.6% yield) as a yellow solid. LCMS: m/z=414.1 [M+H]⁺. ¹H NMR: (500 MHz, CDCl₃) δ: 0.99 (t, J=7.5 Hz, 3H), 1.39 (d, J=6.5 Hz, 3H), 1.52 (s, 3H), 1.76-1.69 (m, 1H), 1.84-1.76 (m, 1H), 1.93-1.91 (m, 2H), 2.05-2.10 (m, 2H), 4.04 (s, 2H), 5.38-5.33 (m, 1H), 7.07 (s, 1H), 8.46 (s, 1H).

### Preparation 398: rac-methyl (R)-7-(sec-butoxy)-2-(1-methyl-2-oxabicyclo[2.1.1]hexan-4-yl)imidazo[1,2-a]pyrimidine-6-carboxylate

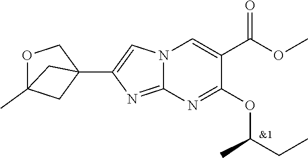

To a solution of rac-(R)-7-(sec-butoxy)-6-iodo-2-(1-methyl-2-oxabicyclo[2.1.1]hexan-4-yl)imidazo[1,2-a]pyrimidine (270 mg, 653 μmol, 1.0 eq.) in MeOH (15.00 mL) was added TEA (661.1 mg, 6.53 mmol, 910.6 μL, 10.0 eq.) and Pd(dppf)Cl₂ (47.8 mg, 65.3 μmol, 0.1 eq.) at 20° C. under Argon. The mixture was stirred at 80° C. under carbon monoxide (50 psi) for 14 hours. The reaction was evaporated under vacuum to give the residue. The residue was purified by Combi-Flash (PE: EtOAc from 3:1 to 1:1) to give rac-methyl (R)-7-(sec-butoxy)-2-(1-methyl-2-oxabicyclo[2.1.1]hexan-4-yl)imidazo[1,2-a]pyrimidine-6-carboxylate (170 mg, 67.8% yield) as a yellow solid. LCMS: m/z=346.3 [M+H]⁺. ¹H NMR: (400 MHz, CDCl₃) δ: 0.99 (t, J=7.2 Hz, 3H), 1.40 (d, J=6.4 Hz, 3H), 1.53 (s, 3H), 1.78-1.69 (m, 1H), 1.87-1.79 (m, 1H), 1.94-1.92 (m, 2H), 2.11-2.08 (m, 2H), 3.92 (s, 3H), 4.05 (s, 2H), 5.49-5.44 (m, 1H), 7.16 (s, 1H), 8.84 (s, 1H).

### Preparation 399: rac-(R)-7-(sec-butoxy)-2-(1-methyl-2-oxabicyclo[2.1.1]hexan-4-yl)imidazo[1,2-a]pyrimidine-6-carboxylic acid

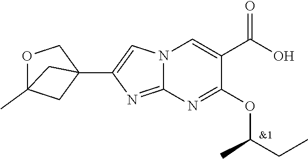 [SMILES: CC[C@@H](C)Oc1nc2nc(C34COC(C)(C3)C4)cn2cc1C(=O)O]

To a solution of rac-methyl (R)-7-(sec-butoxy)-2-(1-methyl-2-oxabicyclo[2.1.1]hexan-4-yl)imidazo[1,2-a]pyrimidine-6-carboxylate (170 mg, 492 μmol, 1.0 eq.) in MeOH (2 mL) and water (2 mL) was added NaOH (39.4 mg, 984 μmol, 2.0 eq.) at 20° C. The reaction was stirred at 20° C. for 14 hours. MeOH was evaporated under vacuum. The mixture was acidfied with aqueous KHSO₄ to pH<7 and evaporated under vacuum to give rac-(R)-7-(sec-butoxy)-2-(1-methyl-2-oxabicyclo[2.1.1]hexan-4-yl)imidazo[1,2-a]pyrimidine-6-carboxylic acid (150 mg, 82.8% yield) as a white solid. LCMS: m/z=332.3 [M+H]⁺. ¹H NMR: (400 MHz, DMSO-d₆) δ: 0.93 (t, J=7.6 Hz, 3H), 1.29 (d, J=6.0 Hz, 3H), 1.41 (s, 3H), 1.69-1.61 (m, 2H), 1.75-1.70 (m, 2H), 1.96-1.95 (m, 2H), 3.84 (s, 2H), 5.19-5.13 (m, 1H), 7.50 (s, 1H), 8.94 (s, 1H).

### Preparation 400: rac-(R)-7-(sec-butoxy)-2-(1-(fluoromethyl)-2-oxabicyclo[2.1.1]hexan-4-yl)-6-iodoimidazo[1,2-a]pyrimidine

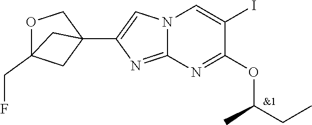 [SMILES: CC[C@@H](C)Oc1nc2nc(C34COC(CF)(C3)C4)cn2cc1I]

To a solution of rac-(R)-4-(sec-butoxy)-5-iodopyrimidin-2-amine (preparation 396; 200 mg, 682 μmol, 1.0 eq.) in tert-Butanol (10.00 mL) was added NaHCO₃ (114.65 mg, 1.36 mmol, 2.0 eq.) and 2-bromo-1-(1-(fluoromethyl)-2-oxabicyclo[2.1.1]hexan-4-yl)ethan-1-one (preparation 166; 200 mg, 844 μmol, 1.24 eq.) at 20° C. The reaction was stirred at 90° C. for 14 hours. The reaction was evaporated under vacuum. The residue was purified by Comb-Flash (PE: EA from 3:1 to 1:1) to give rac-(R)-7-(sec-butoxy)-2-(1-(fluoromethyl)-2-oxabicyclo[2.1.1]hexan-4-yl)-6-iodoimidazo[1,2-a]pyrimidine (160 mg, 48.9% yield) as a yellow solid. LCMS: m/z=432.1 [M+H]⁺. ¹H NMR: (400 MHz, CDCl₃) δ: 0.99 (t, J=7.6 Hz, 3H), 1.39 (d, J=6.4 Hz, 3H), 1.76-1.68 (m, 1H), 1.85-1.78 (m, 1H), 2.04-2.02 (m, 2H), 2.24-2.22 (m, 2H), 4.11 (s, 2H), 4.75-4.63 (m, 2H), 5.38-5.33 (m, 1H), 7.11 (s, 1H), 8.48 (s, 1H).

### Preparation 401: rac-Methyl (R)-7-(sec-butoxy)-2-(1-(fluoromethyl)-2-oxabicyclo[2.1.1]hexan-4-yl)imidazo[1,2-a]pyrimidine-6-carboxylate

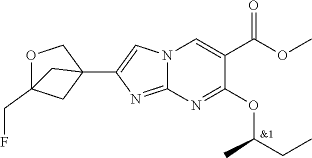 [SMILES: CC[C@@H](C)Oc1nc2nc(C34COC(CF)(C3)C4)cn2cc1C(=O)OC]

To a solution of rac-(R)-7-(sec-butoxy)-2-(1-(fluoromethyl)-2-oxabicyclo[2.1.1]hexan-4-yl)-6-iodoimidazo[1,2-a]pyrimidine (preparation 400, 160 mg, 371 μmol, 1.0 eq.) in MeOH (10.00 mL) was added TEA (375.4 mg, 3.71 mmol, 517.1 μL, 10.0 eq.) and Pd(dppf)Cl₂ (27.1 mg, 37.1 μmol, 0.1 eq.) at 20° C. under Argon. The mixture was stirred at 80° C. under carbon monoxide (50 psi) for 14 hours. The reaction was evaporated under vacuum to give the residue. The residue was purified by Combi-Flash (PE: EtOAc from 3:1 to 1:1) to give rac-methyl (R)-7-(sec-butoxy)-2-(1-(fluoromethyl)-2-oxabicyclo[2.1.1]hexan-4-yl)imidazo[1,2-a]pyrimidine-6-carboxylate (110 mg, 73.4% yield) as a yellow solid. LCMS: m/z=364.2 [M+H]⁺. ¹H NMR: (400 MHz, CDCl₃) δ: 1.00 (t, J=7.6 Hz, 3H), 1.41 (d, J=6.0 Hz, 3H), 1.78-1.69 (m, 1H), 1.87-1.78 (m, 1H), 2.04-2.02 (m, 2H), 2.26-2.24 (m, 2H), 3.93 (s, 3H), 4.13 (s, 2H), 4.76-4.63 (m, 2H), 5.50-5.44 (m, 1H), 7.20 (s, 1H), 8.85 (s, 1H).

### Preparation 402: rac-(R)-7-(sec-butoxy)-2-(1-(fluoromethyl)-2-oxabicyclo[2.1.1]hexan-4-yl)imidazo[1,2-a]pyrimidine-6-carboxylic acid

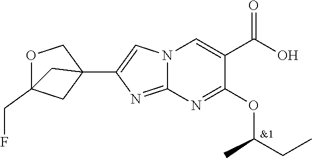 [SMILES: CC[C@@H](C)Oc1nc2nc(C34COC(CF)(C3)C4)cn2cc1C(=O)O]

To a solution of rac-methyl (R)-7-(sec-butoxy)-2-(1-(fluoromethyl)-2-oxabicyclo[2.1.1]hexan-4-yl)imidazo[1,2-a]pyrimidine-6-carboxylate (110.0 mg, 302.7 μmol, 1.0 eq.) in MeOH (3 mL) and water (3 mL) was added NaOH (24.2 mg, 605 μmol, 2.0 eq.) at 20° C. The reaction was stirred at 20° C. for 14 hours. MeOH was evaporated under vacuum. The mixture was added with aqueous KHSO₄ to pH<7 and evaporated under vacuum to give rac-(R)-7-(sec-butoxy)-2-(1-(fluoromethyl)-2-oxabicyclo[2.1.1]hexan-4-yl)imidazo[1,2-a]pyrimidine-6-carboxylic acid (90 mg, 76% yield, 90%) as a white solid. LCMS: m/z=350.2 [M+H]t ¹H NMR: (400 MHz, DMSO-d₆) δ: 0.93 (t, J=7.2 Hz, 3H), 1.29 (d, J=6.4 Hz, 3H), 1.72-1.60 (m, 2H), 1.82-1.80 (m, 2H), 2.11-2.09 (m, 2H), 3.92 (s, 2H), 4.75-4.62 (m, 2H), 5.18-5.12 (m, 1H), 7.53 (s, 1H), 8.84 (s, 1H).

### Preparation 403: rac-Isopropyl 2-((1R,5R)-2,6-dioxabicyclo[3.2.1]octan-1-yl)-′7-isopropoxyimidazo[1,2-a]pyridine-6-carboxylate

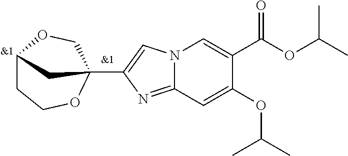 [SMILES: CC(C)OC(=O)c1cn2cc([C@@]34CO[C@@H](CCO3)C4)nc2cc1OC(C)C]

To a solution of isopropyl 6-amino-4-isopropoxynicotinate (preparation 182; 100.0 mg, 0.4197 mmol, 1.0 eq.) and rac-1-((1R,5S)-2,6-dioxabicyclo[3.2.1]octan-1-yl)-2-bromoethan-1-one (118.4 mg, 0.5036 mmol, 1.2 eq.) in t-BuOH (5.00 mL) was added NaHCO₃ (70.5 mg, 0.839 mmol, 2.0 eq.). The mixture was stirred at 90° C. for 16 h. The reaction mixture concentrated to give the residue. The residue was purified by combi-flash (PE/EA=1/1) to give rac-isopropyl 2-((1R,5R)-2,6-dioxabicyclo[3.2.1]octan-1-yl)-7-isopropoxyimidazo[1,2-a]pyridine-6-carboxylate (220.0 mg, crude) as yellow oil. ¹H NMR: (500 MHz, CDCl₃) δ: 1.37 (d, J=6.0 Hz, 6H), 1.42 (d, J=6.5 Hz, 6H), 1.65-1.53 (m, 2H), 1.80-1.73 (m, 1H), 1.89-1.80 (m, 1H), 4.13 (q, J=7.0 Hz, 1H), 4.26-4.18 (m, 2H), 4.37 (d, J=9.5 Hz, 1H), 4.67-4.57 (m, 1H), 4.75 (t, J=6.0 Hz, 1H), 5.25 (t, J=6.0 Hz, 1H), 6.87 (s, 1H), 7.43 (s, 1H), 8.53 (s, 1H).

### Preparation 404: rac-2-((1R,5R)-2,6-dioxabicyclo[3.2.1]octan-1-yl)-′7-isopropoxyimidazo[1,2-a]pyridine-6-carboxylic acid

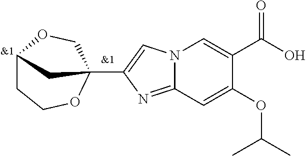 [SMILES: CC(C)Oc1cc2nc([C@@]34CO[C@@H](CCO3)C4)cn2cc1C(=O)O]

To a solution of rac-isopropyl 2-((1R,5R)-2,6-dioxabicyclo[3.2.1]octan-1-yl)-7-isopropoxyimidazo[1,2-a]pyridine-6-carboxylate (220.0 mg, crude) in MeOH (1.00 mL) and water (1.00 mL) was added NaOH (70.5 mg, 1.76 mmol, 3.0 eq.). The mixture was stirred at 20° C. for 16 h. The mixture was adjusted by HCl aq. (1 M) to pH=3 and concentrated in vacuo to give rac-2-((1R,5R)-2,6-dioxabicyclo[3.2.1]octan-1-yl)-7-isopropoxyimidazo[1,2-a]pyridine-6-carboxylic acid (320.0 mg, crude) as a yellow solid. LCMS: m/z=332.9 [M+H]⁺.

### Preparation 405: rac-2-((1R,5R)-2,6-dioxabicyclo[3.2.1]octan-1-yl)-6-iodo-7-isopropoxyimidazo[1,2-a]pyrimidine

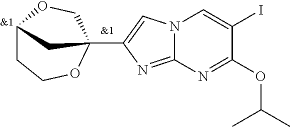 [SMILES: CC(C)Oc1nc2nc([C@@]34CO[C@@H](CCO3)C4)cn2cc1I]

To a mixture of 5-iodo-4-isopropoxy-pyrimidin-2-amine (preparation 196; 200.0 mg, 716.6 μmol) and 2-bromo-1-(4,7-dioxabicyclo[3.2.1]octan-5-yl)ethanone (200.5 mg, 852.8 μmol) in t-BuOH (10.00 mL) was added NaHCO₃ (120.4 mg, 1.43 mmol, 55.8 μL). The mixture was stirred at 100° C. for 16 h. The mixture was concentrated in vacuo to give the residue, which was purified by Combi Flash (PE/EtOAc=1/1) to give 2-(4,7-dioxabicyclo[3.2.1]octan-5-yl)-6-iodo-7-isopropoxy-imidazo[1,2-a]pyrimidine (270 mg, 88.9% yield) as a white solid. LCMS: m/z=416.1 [M+H]⁺. ¹H NMR: (400 MHz, CDCl₃) δ: 1.43 (d, J=6.0 Hz, 6H), 1.85-1.77 (m, 2H), 1.97 (d, J=11.6 Hz, 1H), 2.64-2.62 (m, 1H), 4.28-4.05 (m, 4H), 4.74-4.72 (m, 1H), 5.54-5.49 (m, 1H), 7.26 (s, 1H), 8.47 (s, 1H).

### Preparation 406: rac-Methyl 2-((1R,5R)-2,6-dioxabicyclo[3.2.1]octan-1-yl)-′7-isopropoxyimidazo[1,2-a]pyrimidine-6-carboxylate

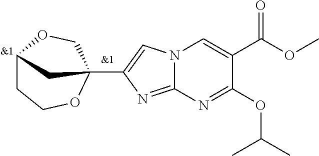 [SMILES: COC(=O)c1cn2cc([C@@]34CO[C@@H](CCO3)C4)nc2nc1OC(C)C]

To a solution of 2-(4,7-dioxabicyclo[3.2.1]octan-5-yl)-6-iodo-7-isopropoxy-imidazo[1,2-a]pyrimidine (270.0 mg, 650.2 μmol) in MeOH (10.00 mL) was added TEA (658.0 mg, 6.50 mmol, 901.3 μL) and Pd(dppf)Cl₂ (47.6 mg, 65.0 μmol). The mixture was degassed with CO for 3 times and it was stirred at 80° C. under CO (50 psi) for 16 h. The mixture was concentrated in vacuo to give the residue, which was purified by Combi Flash (PE/EtOAc=1/1) to give methyl 2-(4,7-dioxabicyclo[3.2.1]octan-5-yl)-7-isopropoxy-imidazo[1,2-a]pyrimidine-6-carboxylate (175.0 mg, 75.9% yield) as a brown solid. LCMS: m/z=348.3 [M+H]t ¹H NMR: (400 MHz, CDCl₃) δ: 1.43 (d, J=6.0 Hz, 6H), 1.83-1.77 (m, 2H), 1.96 (d, J=11.6 Hz, 1H), 2.65-2.61 (m, 1H), 3.91 (s, 3H), 4.09-4.07 (m, 1H), 4.31-4.20 (m, 3H), 4.75-4.72 (m, 1H), 5.63-5.59 (m, 1H), 7.35 (s, 1H), 8.84 (s, 1H).

### Preparation 407: rac-2-((1R,5R)-2,6-dioxabicyclo[3.2.1]octan-1-yl)-′7-isopropoxyimidazo[1,2-a]pyrimidine-6-carboxylic acid

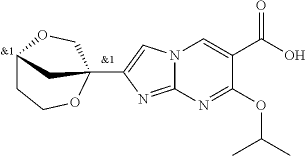 [SMILES: CC(C)Oc1nc2nc([C@@]34CO[C@@H](CCO3)C4)cn2cc1C(=O)O]

To a solution of rac-methyl 24(1R,5R)-2,6-dioxabicyclo[3.2.1]octan-1-yl)-′7-isopropoxyimidazo[1,2-a]pyrimidine-6-carboxylate (175.0 mg, 503.8 μmol) in MeOH (3.00 mL) and water (3.00 mL) was added NaOH (60.5 mg, 1.51 mmol, 3.0 eq.). The mixture was stirred at 20° C. for 16 h. The mixture was adjusted by HCl aq. (1 M) to pH=3 and concentrated in vacuo to give a residual, which was recrystallized from water, dried by lyophilization to afford rac-2-((1R,5R)-2,6-dioxabicyclo[3.2.1]octan-1-yl)-7-isopropoxyimidazo[1,2-a]pyrimidine-6-carboxylic acid_(160.0 mg, 92.4% yield) as a brown solid. LCMS: m/z=333.9 [M+H]⁺.

### Preparation 408: 5-bromo-4-cyclobutoxypyrimidin-2-amine

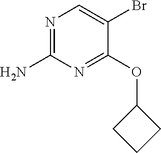 [SMILES: Nc1ncc(Br)c(OC2CCC2)n1]

To a solution of 4-cyclobutoxypyrimidin-2-amine (preparation 175; 28.0 g, 170 mmol) in CHCl₃ (300 mL) was added NBS (30.3 g, 170 mmol) in portions at 10° C. The resulting mixture was stirred at r.t. for 2 h and diluted with water. The organic layer was washed with water, brine, dried over Na₂SO₄ and evaporated in vacuo to afford 5-bromo-4-cyclobutoxypyrimidin-2-amine (37.2 g, 90% yield).

### Preparation 409: methyl 2-amino-4-cyclobutoxypyrimidine-5-carboxylate

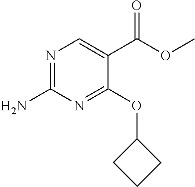 [SMILES: COC(=O)c1cnc(N)nc1OC1CCC1]

To a stirred mixture of 5-bromo-4-cyclobutoxypyrimidin-2-amine (37.2 g, 152 mmol) in MeOH (600 mL) in a steel bomb were added Pd(dppf)Cl₂ (2.49 g, 0.3 mmol), triethylamine (18.5 g, 183 mmol) at room temperature and then the steel vessel was closed tightly. Then CO gas was purged into the steel bomb and the stirring was continued at 120° C. for 18 hours. The reaction mixture was allowed to warm up to room temperature, filtered through a pad of celite. The celite pad was washed with excess of methanol and the filtrate was concentrated under vacuum. The residue was washed with water, rinsed with MeOH and dried to afford methyl 2-amino-4-cyclobutoxypyrimidine-5-carboxylate (27.1 g, 80% yield).

### Preparation 410: 2-amino-4-cyclobutoxypyrimidine-5-carboxylic acid

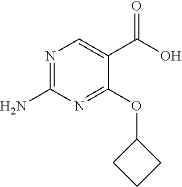 [SMILES: Nc1ncc(C(=O)O)c(OC2CCC2)n1]

To a suspension of methyl 2-amino-4-cyclobutoxypyrimidine-5-carboxylate (10.7 g, 47.8 mmol) in MeOH was added aq. solution of NaOH (2.87 g, 71.7 mmol in 50 mL of water). The mixture was heated to 50° C. and stirred for 5 h. Upon completion of the reaction, the mixture was concentrated. The residue was diluted with water and acidified with citric acid. The precipitated solid was collected, washed with water, rinsed with MeOH and dried to afford 2-amino-4-cyclobutoxypyrimidine-5-carboxylic acid (6.3 g, 63%).

### Preparation 411: 2-amino-4-cyclobutoxy-N-(1-methyl-1H-pyrazol-3-yl)pyrimidine-5-carboxamide

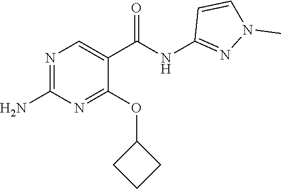 [SMILES: Cn1ccc(NC(=O)c2cnc(N)nc2OC2CCC2)n1]

2-Amino-4-cyclobutoxypyrimidine-5-carboxylic acid (preparation 410; 0.599 g, 2.90 mmol), 1-methyl-1H-pyrazol-3-amine (0.253 g, 2.60 mmol) and 3H-[1,2,3]triazolo[4,5-b]pyridine-3-ol (0.389 g, 2.90 mmol) were mixed in DMA (4 mL) and the reaction mixture was stirred at −10° C. for 10 min. Then EDCE (0.485 g, 3.10 mmol) was added and the resulting mixture was stirred at r.t. overnight. Upon completion the mixture was poured into water. The precipitated solid was collected by filtration, washed with water and dried in vacuo to give amino-4-cyclobutoxy-N-(1-methyl-1H-pyrazol-3-yl)pyrimidine-5-carboxamide (0.355 g, 47.0% yield). LCMS: m/z=289.0 [M+H]⁺.

### PREPARATION OF EXAMPLES

### Example 1: 7-Methoxy-2-(1-methyl-2-oxabicyclo[2.1.1]hexan-4-yl)-N-(2-pyridyl)imidazo[1,2-a]pyridine-6-carboxamide

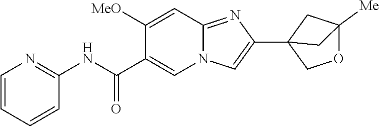 [SMILES: COc1cc2nc(C34COC(C)(C3)C4)cn2cc1C(=O)Nc1ccccn1]

A 1:1 mixture of PrCN/toluene (2 mL) was added to a vial containing 6-amino-4-methoxy-N-(pyridin-2-yl)nicotinamide trifluoroacetate (Preparation 93, 104 mg, 0.128 mmol), 2-chloro-1-(1-methyl-2-oxabicyclo[2.1.1]hexan-4-yl)ethan-1-one (Preparation 6, 33.5 mg, 0.192 mmol) and NaHCO₃ (107 mg, 1.28 mmol). The vial was sealed and heated at 100° C. for 18 h. The cooled reaction mixture was filtered through a pad of Celite® and the filtrate evaporated to dryness in vacuo. The residue was purified by prep HPLC (SunFire C18 column, 60 mL/min flow rate, MeCN/H₂O/0.1% TFA; Gradient (% organic): 10-70) to afford 7-methoxy-2-(1-methyl-2-oxabicyclo[2.1.1]hexan-4-yl)-N-(2-pyridyl)imidazo[1,2-a]pyridine-6-carboxamideas a white solid (8.2 mg, 17% yield). LCMS m/z=365 [M+H]⁺; ¹H NMR (400 MHz, MeOH-d₄) δ: 1.45-1.59 (m, 3H), 1.90 (dd, 2H), 2.09-2.18 (m, 2H), 3.96-4.07 (m, 2H), 4.18 (s, 3H), 7.02 (s, 1H), 7.14-7.25 (m, 1H), 7.72 (s, 1H), 7.88 (ddd, 1H), 8.31-8.41 (m, 2H), 9.11 (s, 1H).

### Examples 2-49

The title compounds were prepared in an analogous manner to that described for Example 1 using either 6-amino-4-methoxy-N-(pyridin-2-yl)nicotinamide trifluoroacetate (Preparation 93) in PrCN/Toluene (1:1) (Amine A), 6-amino-4-methoxy-N-(6-methoxypyridin-2-yl)nicotinamide trifluoroacetate (Preparation 95) in PrCN/dioxane (Amine B), 6-amino-4-methoxy-N-(6-(trifluoromethyl)pyridin-2-yl)nicotinamide (Preparation 92) in EtOH (Amine C) or 6-amino-N-(6-methoxypyridin-2-yl)nicotinamide trifluoroacetate (Preparation 97) in EtOH (Amine D), 6-amino-N-(1-(difluoromethyl)-1H-pyrazol-3-yl)-4-methoxynicotinamide trifluoroacetate (Preparation 96, Amine E), 6-amino-N-(1-(difluoromethyl)-1H-pyrazol-3-yl)nicotinamide trifluoroacetate (Preparation 98, Amine F) and the appropriate ketone as shown in the following table. Compounds purified by prep-HPLC (SunFire C18 column, 60 mL/min flow rate, MeCN/H₂O/0.1% TFA; Gradient (% organic): 0-100% optimized for individual separations)

| Example | Name/Structure/Amine/Ketone | QC Data |
|---|---|---|
| 2 | 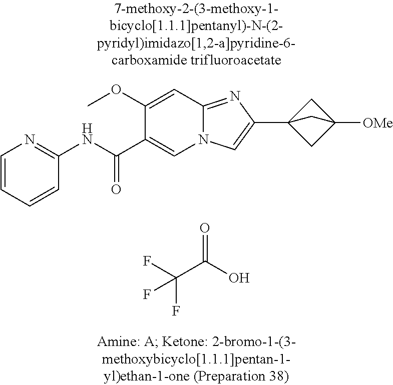 [SMILES: COc1cc2nc(C34CC(OC)(C3)C4)cn2cc1C(=O)Nc1ccccn1 ; O=C(O)C(F)(F)F] | White solid (12.3 mg, 26%). LCMS m/z = 365 [M + H]⁺ ¹H NMR (500 MHz, DMSO-d₆) δ: 2.24-2.36 (m, 7H), 3.24-3.39 (m, 2H), 4.07 (s, 3H), 7.18-7.27 (m, 2H), 7.86-7.93 (m, 1H), 7.93-8.03 (m, 1H), 8.21 (br d, 1H), 8.33-8.43 (m, 1H), 9.11 (s, 1H), 10.80 (br s, 1H) |
| 3 | 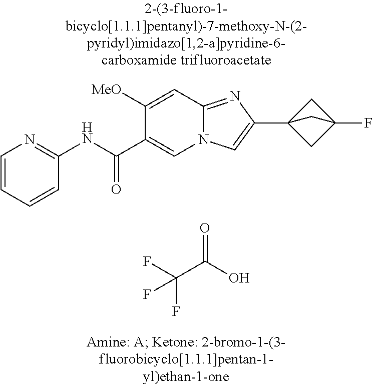 [SMILES: COc1cc2nc(C34CC(F)(C3)C4)cn2cc1C(=O)Nc1ccccn1 ; O=C(O)C(F)(F)F] | White solid (12.1 mg, 27%). LCMS m/z = 353 [M + H]⁺ ¹H NMR (500 MHz, DMSO-d₆) δ: 2.51-2.58 (m, 6H), 3.97-4.13 (m, 3H), 7.17-7.28 (m, 2H), 7.81-7.93 (m, 1H), 7.98 (br s, 1H), 8.21 (br d, 1H), 8.39 (br d, 1H), 9.11 (s, 1H), 10.77 (br s, 1H) |
| 4 | 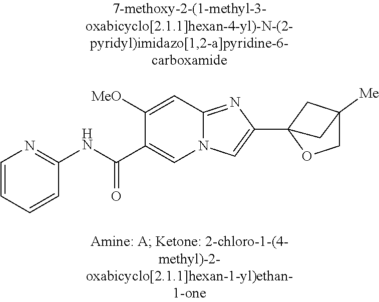 [SMILES: COc1cc2nc(C34CC(C)(CO3)C4)cn2cc1C(=O)Nc1ccccn1] | White solid (7 mg, 15%). LCMS m/z = 365 [M + H]⁺ ¹H NMR (500 MHz, DMSO-d₆) δ: 1.32-1.40 (m, 4H), 1.66-1.77 (m, 3H), 1.93-2.04 (m, 3H), 3.56-3.67 (m, 2H), 3.94-4.04 (m, 4H), 7.08 (s, 1H), 7.12-7.23 (m, 1H), 7.77-7.92 (m, 2H), 8.11-8.26 (m, 1H), 8.29- 8.44 (m, 1H), 9.05 (s, 1H), 10.52 (br s, 1H) |
| 5 | 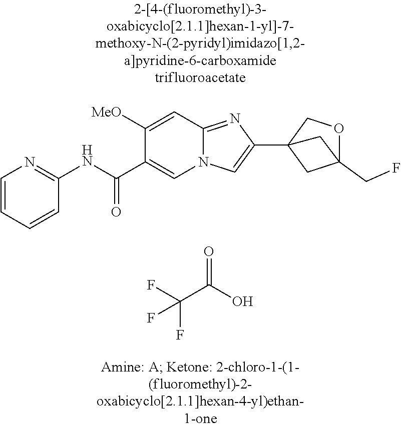 [SMILES: COc1cc2nc(C34COC(CF)(C3)C4)cn2cc1C(=O)Nc1ccccn1 ; O=C(O)C(F)(F)F] | White solid (7.9 mg, 16%). LCMS m/z = 383 [M + H]⁺ ¹H NMR (500 MHz, DMSO-d₆) δ: 1.91-2.03 (m, 2H), 2.28 (br d, 2H), 3.98-4.16 (m, 4H), 4.64-4.84 (m, 2H), 7.19-7.29 (m, 2H), 7.86-7.96 (m, 1H), 8.06 (br s, 1H), 8.22 (br d, 1H), 8.34-8.46 (m, 1H), 9.13 (s, 1H), 10.80 (br s, 1H) |
| 6 | 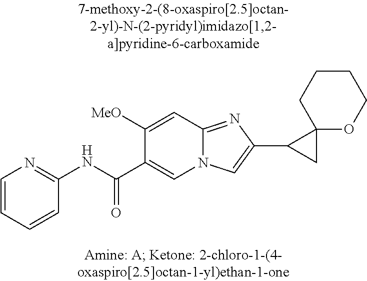 [SMILES: COc1cc2nc(C3CC34CCCCO4)cn2cc1C(=O)Nc1ccccn1] | White solid (4.2 mg, 8.7%). LCMS m/z = 379 [M + H]⁺ ¹H NMR (500 MHz, DMSO-d₆) δ: 0.90-1.00 (m, 1H), 1.00-1.11 (m, 1H), 1.33- 1.41 (m, 2H), 1.44-1.59 (m, 4H), 1.99 (dd, 1H), 3.62-3.74 (m, 4H), 3.97-4.09 (m, 3H), 7.10 (s, 1H), 7.19 (dd, 1H), 7.76 (s, 1H), 7.84-7.93 (m, 1H), 8.23 (br d, 1H), 8.38 (br d, 1H), 8.99 (s, 1H), 10.50 (br s, 1H) |
| 7 | 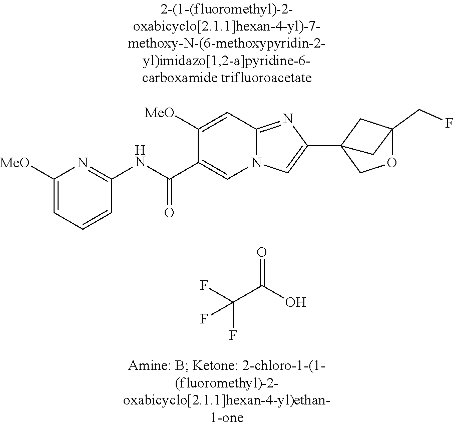 [SMILES: COc1cccc(NC(=O)c2cn3cc(C45COC(CF)(C4)C5)nc3cc2OC)n1 ; O=C(O)C(F)(F)F] | White solid (1.6 mg, 2%). LCMS m/z = 413 [M + H]⁺ |
| 8 | 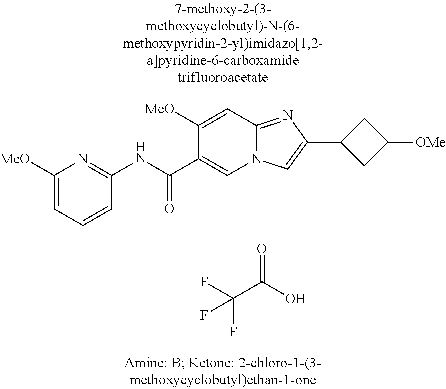 [SMILES: COc1cccc(NC(=O)c2cn3cc(C4CC(OC)C4)nc3cc2OC)n1 ; O=C(O)C(F)(F)F] | Yellow oil (1.7 mg, 2%). LCMS m/z = 383 [M + H]⁺ |
| 9 | 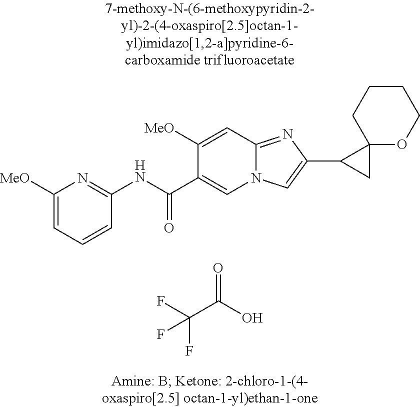 [SMILES: COc1cccc(NC(=O)c2cn3cc(C4CC45CCCCO5)nc3cc2OC)n1 ; O=C(O)C(F)(F)F] | Yellow oil (1.8 mg, 2%). LCMS m/z = 409 [M + H]⁺ |
| 10 | 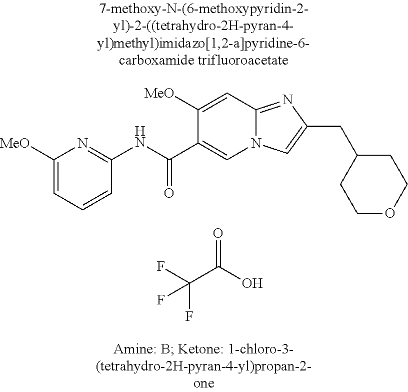 [SMILES: COc1cccc(NC(=O)c2cn3cc(CC4CCOCC4)nc3cc2OC)n1 ; O=C(O)C(F)(F)F] | White solid (0.9 mg, 1%). LCMS m/z = 397 [M + H]⁺ |
| 11 | 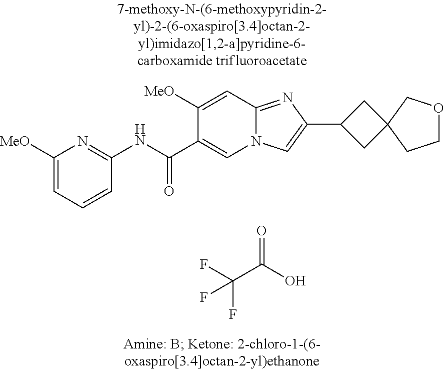 [SMILES: COc1cccc(NC(=O)c2cn3cc(C4CC5(CCOC5)C4)nc3cc2OC)n1 ; O=C(O)C(F)(F)F] | Yellow oil (1.4 mg, 1.5%). LCMS m/z = 409 [M + H]⁺ |
| 12 | 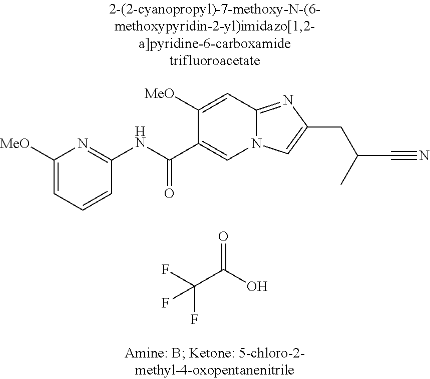 [SMILES: COc1cccc(NC(=O)c2cn3cc(CC(C)C#N)nc3cc2OC)n1 ; O=C(O)C(F)(F)F] | Yellow oil (1.5 mg, 2%). LCMS m/z = 366 [M + H]⁺ |
| 13 | 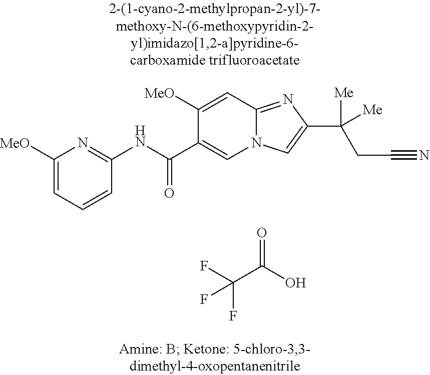 [SMILES: COc1cccc(NC(=O)c2cn3cc(C(C)(C)CC#N)nc3cc2OC)n1 ; O=C(O)C(F)(F)F] | Yellow oil (0.5 mg, 0.6%). LCMS m/z = 380 [M + H]⁺ |
| 14 | 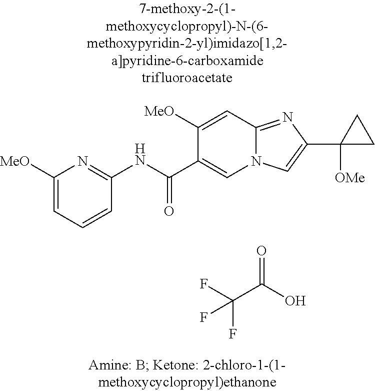 [SMILES: COc1cccc(NC(=O)c2cn3cc(C4(OC)CC4)nc3cc2OC)n1 ; O=C(O)C(F)(F)F] | White solid (2.7 mg, 4%). LCMS m/z = 369 [M + H]⁺ ¹H NMR (500 MHz, DMSO-d₆) δ: 1.02-1.19 (m, 3H), 1.21-1.34 (m, 3H), 3.17-3.60 (m, 2H), 3.61-3.91 (m, 2H), 3.94-4.12 (m, 4H), 6.56- 6.66 (m, 1H), 7.14 (s, 1H) 7.69-7.85 (m, 2H), 8.03 (s, 1H), 9.07 (s, 1H), 10.57 (br s, 1H). |
| 15 | 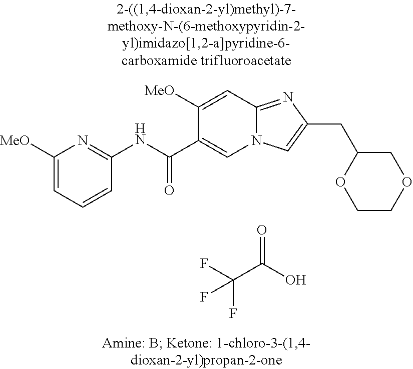 [SMILES: COc1cccc(NC(=O)c2cn3cc(CC4COCCO4)nc3cc2OC)n1 ; O=C(O)C(F)(F)F] | White solid (5.8 mg, 8%). LCMS m/z = 399 [M + H]⁺ ¹H NMR (500 MHz, DMSO-d₆) δ: 2.79-2.92 (m, 1H), 2.92-3.02 (m, 1H), 3.25-3.92 (m, 9H), 4.01-4.14 (m, 3H), 6.55-6.70 (m, 1H), 7.29 (s, 1H), 7.78 (br d, 2H), 7.98 (s, 1H), 9.15 (s, 1H), 10.67 (br s, 1H). |
| 16 | 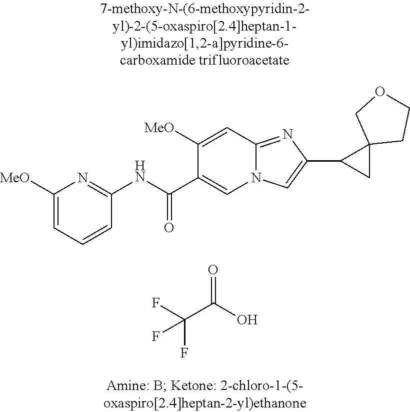 [SMILES: COc1cccc(NC(=O)c2cn3cc(C4CC45CCOC5)nc3cc2OC)n1 ; O=C(O)C(F)(F)F] | Colourless oil (12.9 mg, 18%). LCMS m/z = 395 [M + H]⁺ ¹H NMR (500 MHz, DMSO-d₆) δ: 1.27-1.50 (m, 2H), 1.50-1.88 (m, 1H), 2.03 (t, 2H), 2.27-2.43 (m, 1H), 3.21-3.75 (m, 3H), 3.75-3.91 (m, 3H), 3.98-4.14 (m, 3H), 6.57-6.68 (m, 1H), 7.25 (s, 1H), 7.71-7.81 (m, 2H), 7.83-7.96 (m, 1H), 8.99-9.12 (m, 1H), 10.65 (br s, 1H). |
| 17 | 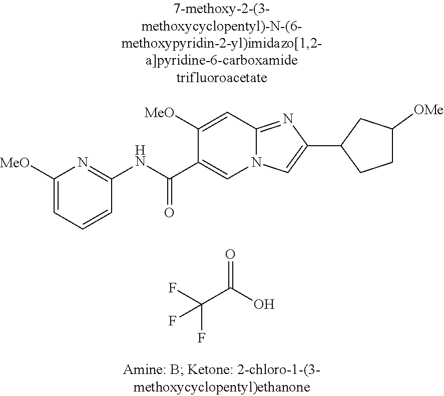 [SMILES: COc1cccc(NC(=O)c2cn3cc(C4CCC(OC)C4)nc3cc2OC)n1 ; O=C(O)C(F)(F)F] | Pale yellow oil (8.8 mg, 12%). LCMS m/z = 397 [M + H]⁺ ¹H NMR (500 MHz, DMSO-d₆) δ: 1.57-1.87 (m, 4H), 1.94-2.27 (m, 3H), 2.33- 2.49 (m, 1H), 3.17-3.27 (m, 2H), 3.33 (s, 2H), 3.39-3.73 (m, 1H), 3.74 (br s, 1H), 3.75-3.88 (m, 1H), 3.88- 4.02 (m, 1H), 4.07 (s, 3H), 6.52-6.71 (m, 1H), 7.24 (s, 1H), 7.78 (br d, 2H), 7.94 (s, 1H), 9.09 (s, 1H), 10.63 (br s, 1H). |
| 18 | 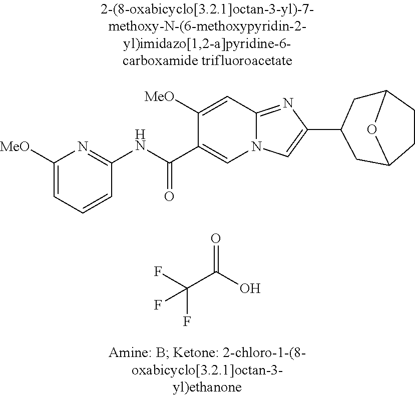 [SMILES: COc1cccc(NC(=O)c2cn3cc(C4CC5CCC(C4)O5)nc3cc2OC)n1 ; O=C(O)C(F)(F)F] | White solid (14.2 mg, 19%). LCMS m/z = 409 [M + H]+ ¹H NMR (500 MHz, DMSO-d₆) δ: 1.68-2.02 (m, 10H), 3.27-3.90 (m, 2H), 3.99- 4.16 (m, 3H), 4.43 (br s, 2H), 6.53- 6.72 (m, 1H), 7.13-7.33 (m, 1H), 7.78 (br d, 2H), 7.91 (s, 1H), 9.03- 9.20 (m, 1H), 10.64 (br s, 1H). |
| 19 | 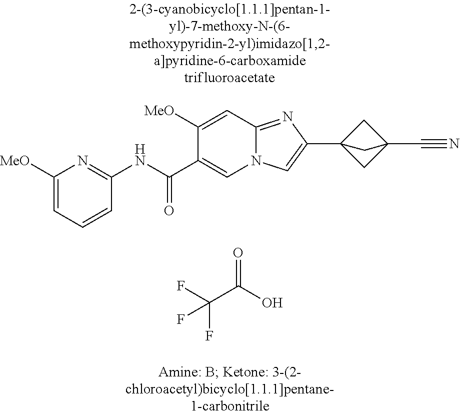 [SMILES: COc1cccc(NC(=O)c2cn3cc(C45CC(C#N)(C4)C5)nc3cc2OC)n1 ; O=C(O)C(F)(F)F] | White solid (13.2 mg, 18%). LCMS m/z = 390 [M + H]⁺ ¹H NMR (500 MHz, DMSO-d₆) δ: 2.65 (s, 6H), 3.27-3.88 (m, 2H), 4.04 (s, 3H), 6.50-6.73 (m, 1H), 7.18 (s, 1H), 7.77 (br d, 2H), 7.94 (br s, 1H), 9.07 (s, 1H), 10.58 (br s, 1H). |
| 20 | 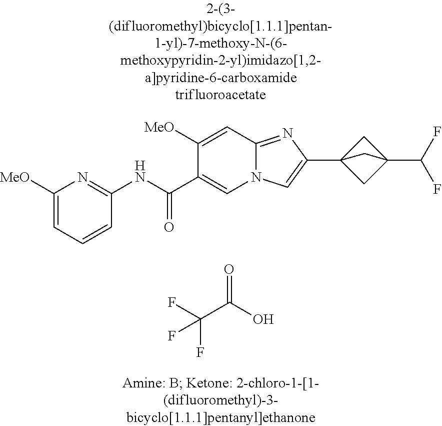 [SMILES: COc1cccc(NC(=O)c2cn3cc(C45CC(C(F)F)(C4)C5)nc3cc2OC)n1 ; O=C(O)C(F)(F)F] | Yellow oil (9.7 mg, 13%). LCMS m/z = 415 [M + H]⁺ ¹H NMR (500 MHz, DMSO-d₆) δ: 2.25 (s, 6H), 3.33-3.89 (m, 3H), 3.97-4.15 (m, 3H), 5.91-6.39 (m, 1H), 6.52-6.72 (m, 1H), 7.21 (s, 1H), 7.78 (br d, 2H), 7.96 (br s, 1H), 9.08 (s, 1H), 10.61 (br s, 1H). |
| 21 | 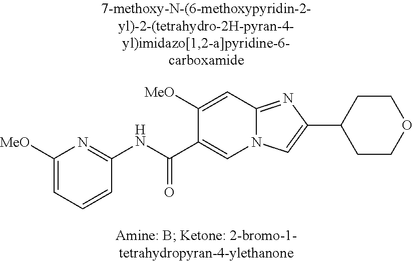 [SMILES: COc1cccc(NC(=O)c2cn3cc(C4CCOCC4)nc3cc2OC)n1] | Yellow oil (45.6 mg, 47%). LCMS m/z = 383 [M + H]⁺ ¹H NMR (400 MHz, MeOH-d₄) δ: 1.79-1.96 (m, 2H), 2.00-2.14 (m, 2H), 3.15-3.27 (m, 1H), 3.63 (td, 2H), 3.92 (s, 3H), 4.04-4.14 (m, 2H), 4.23 (s, 3H), 6.61 (d, 1H), 7.36 (s, 1H), 7.73 (t, 1H), 7.81-7.90 (m, 1H), 7.92 (s, 1H), 9.15 (s, 1H). |
| 22 | 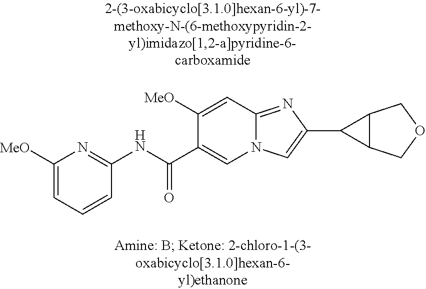 [SMILES: COc1cccc(NC(=O)c2cn3cc(C4C5COCC54)nc3cc2OC)n1] | White solid (1.8 mg, 1%). LCMS m/z = 381 [M + H]⁺ ¹H NMR (400 MHz, MeOH-d₄) δ: 1.87-1.96 (m, 1H), 2.10-2.16 (m, 2H), 3.83 (d, 2H), 3.92 (s, 3H), 4.01 (d, 2H), 4.09-4.17 (m, 3H), 6.58 (d, 1H), 6.96 (s, 1H), 7.59 (s, 1H), 7.67- 7.76 (m, 1H), 7.85 (br d, 1H), 8.98 (s, 1H). |
| 23 | 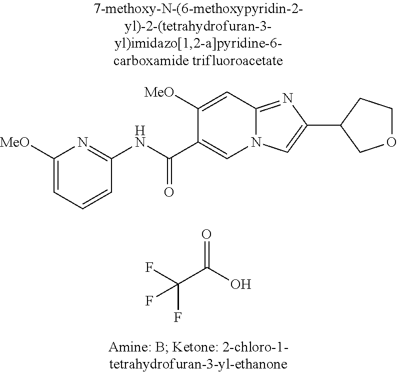 [SMILES: COc1cccc(NC(=O)c2cn3cc(C4CCOC4)nc3cc2OC)n1 ; O=C(O)C(F)(F)F] | White solid (9.6 mg, 13%). LCMS m/z = 369 [M + H]⁺ ¹H NMR (500 MHz, DMSO-d₆) δ: 1.98-2.17 (m, 1H), 2.28-2.41 (m, 1H), 2.85- 3.00 (m, 1H), 3.36 (br s, 1H), 3.58- 3.75 (m, 1H), 3.75-3.97 (m, 5H), 3.98-4.14 (m, 4H), 6.54-6.73 (m, 1H), 6.93-7.20 (m, 1H), 7.23 (s, 1H), 7.78 (br d, 2H), 7.98 (br s, 1H), 9.08 (s, 1H), 10.61 (br s, 1H). |
| 24 | 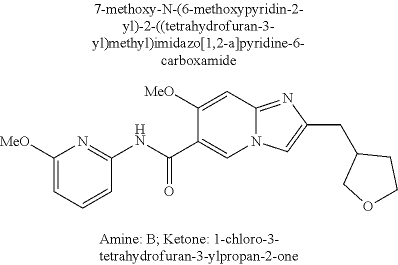 [SMILES: COc1cccc(NC(=O)c2cn3cc(CC4CCOC4)nc3cc2OC)n1] | White solid (9.3 mg, 11%). LCMS m/z = 383 [M + H]⁺ ¹H NMR (400 MHz, MeOH-d₄) δ: 1.66-1.81 (m, 1H), 2.15 (dtd, 1H), 2.65-2.78 (m, 1H), 2.89 (d, 2H), 3.55 (dd, 1H), 3.76-3.86 (m, 1H), 3.86-3.98 (m, 5H), 4.11-4.22 (m, 3H), 6.59 (d, 1H), 7.17 (s, 1H), 7.72 (t, 1H), 7.78 (s, 1H), 7.85 (br d, 1H), 9.08 (s, 1H). |
| 25 | 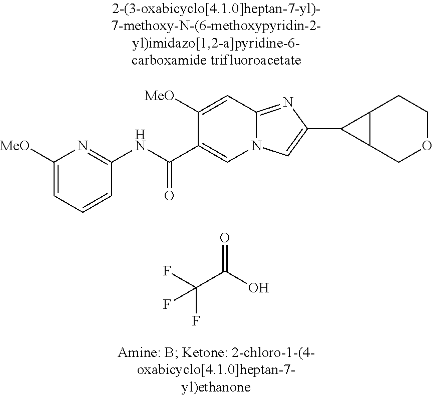 [SMILES: COc1cccc(NC(=O)c2cn3cc(C4C5CCOCC54)nc3cc2OC)n1 ; O=C(O)C(F)(F)F] | White solid (2.1 mg, 3%). LCMS m/z = 395 [M + H]⁺ ¹H NMR (500 MHz, DMSO-d₆) δ: 1.53-1.62 (m, 1H), 1.67 (td, 1H), 1.78-1.87 (m, 1H), 1.94-2.04 (m, 1H), 2.04-2.14 (m, 1H), 3.19-3.61 (m, 2H), 3.78- 4.02 (m, 4H), 4.06 (s, 3H), 6.60-6.66 (m, 1H), 7.19 (s, 1H), 7.78 (br d, 2H), 7.86 (s, 1H), 9.07 (s, 1H), 10.63 (br s, 1H). |
| 26 | 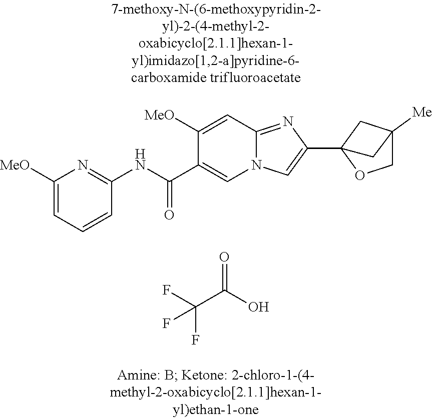 [SMILES: COc1cccc(NC(=O)c2cn3cc(C45CC(C)(CO4)C5)nc3cc2OC)n1 ; O=C(O)C(F)(F)F] | White solid (5.3 mg, 7%). LCMS m/z = 395 [M + H]⁺ ¹H NMR (400 MHz, MeOH-d₄) δ: 1.48 (s, 3H), 1.96-2.00 (m, 2H), 2.14 (d, 2H), 3.80 (s, 3H), 3.92 (s, 3H), 4.22 (s, 3H), 6.62 (d, 1H), 7.27 (s, 1H), 7.70-7.77 (m, 1H), 7.86 (br d, 1H), 8.05 (s, 1H), 9.16 (s, 1H). |
| 27 | 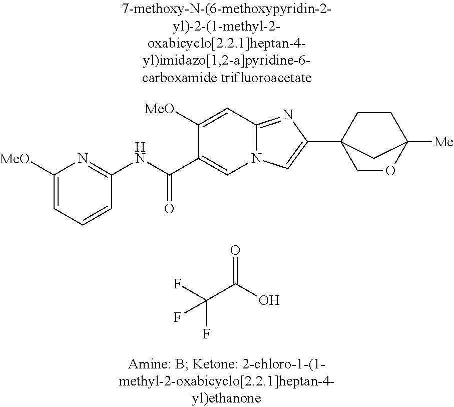 [SMILES: COc1cccc(NC(=O)c2cn3cc(C45CCC(C)(C4)OC5)nc3cc2OC)n1 ; O=C(O)C(F)(F)F] | White solid (1.8 mg, 2%). LCMS m/z = 409 [M + H]⁺ ¹H NMR (400 MHz, MeOH-d₄) δ: 1.40 (s, 4H), 1.81-1.87 (m, 2H), 1.97 (s, 2H), 2.08 (br s, 2H), 3.81 (s, 4H), 3.89 (d, 2H), 3.92-3.96 (m, 2H), 4.13 (s, 3H), 6.51 (d, 1H), 7.19 (s, 1H), 7.63 (t, 1H), 7.74 (s, 1H), 7.87 (s, 1H), 9.03 (s, 1H). |
| 28 | 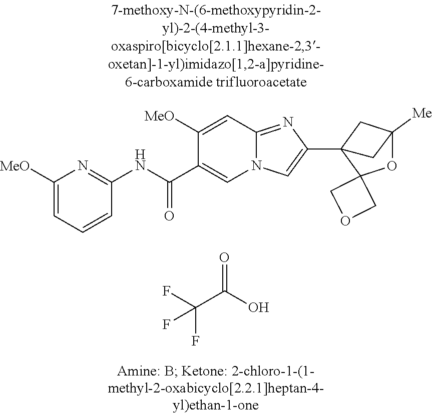 [SMILES: COc1cccc(NC(=O)c2cn3cc(C45CC(C)(C4)OC54COC4)nc3cc2OC)n1 ; O=C(O)C(F)(F)F] | White solid (2.3 mg, 3%). LCMS m/z = 437 [M + H]⁺ ¹H NMR (400 MHz, MeOH-d₄) δ : 1.40 (s, 3H), 1.84-1.89 (m, 2H), 2.14-2.18 (m, 3H), 3.35-3.40 (m, 1H), 3.63 (s, 1H), 3.72 (s, 1H), 3.81 (s, 3H), 3.89 (s, 1H), 4.14 (s, 3H), 6.52 (d, Hz, 1H), 7.24 (s, 1H), 7.63 (t, 1H), 7.76 (br d, 1H), 8.20 (s, 1H), 9.10 (s, 1H). |
| 29 | 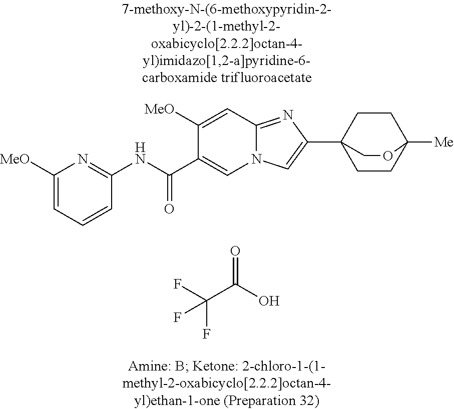 [SMILES: COc1cccc(NC(=O)c2cn3cc(C45CCC(C)(CC4)OC5)nc3cc2OC)n1 ; O=C(O)C(F)(F)F] | White solid (1.8 mg, 2%). LCMS m/z = 423 [M + H]⁺ ¹H NMR (400 MHz, MeOH-d₄) δ: 1.19 (s, 3H), 1.87-1.96 (m, 2H), 1.96-2.04 (m, 2H), 2.09-2.26 (m, 4H), 3.89-3.94 (m, 3H), 4.06- 4.11 (m, 2H), 4.23 (s, 3H), 6.62 (d, 1H), 7.26 (s, 1H), 7.69-7.79 (m, 1H), 7.86 (br d, 1H), 7.90 (s, 1H), 9.13 (s, 1H). |
| 30 | 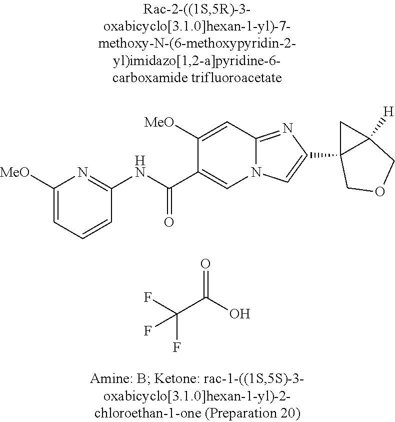 [SMILES: COc1cccc(NC(=O)c2cn3cc([C@@]45COC[C@@H]4C5)nc3cc2OC)n1 ; O=C(O)C(F)(F)F] | White solid (1.0 mg, 1.3%). LCMS m/z = 381 [M + H]⁺ ¹H NMR (400 MHz, MeOH-d₄) δ: 1.17 (q, 1H), 1.29-1.35 (m, 1H), 2.11-2.18 (m, 1H), 3.85 (s, 3H), 3.90-3.96 (m, 3H), 4.07 (d, 1H), 4.16 (s, 3H), 6.55 (d, 1H), 7.22 (s, 1H), 7.64-7.69 (m, 1H), 7.79 (br d, 1H), 7.92 (s, 1H), 9.06 (s, 1H). |
| 31 | 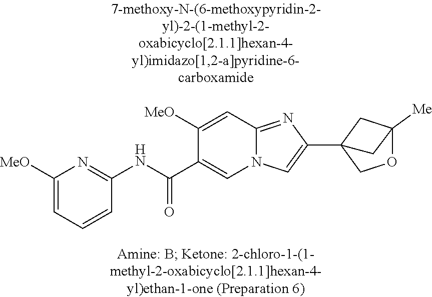 [SMILES: COc1cccc(NC(=O)c2cn3cc(C45COC(C)(C4)C5)nc3cc2OC)n1] | White solid (2.7 mg, 4%). LCMS m/z = 395 [M + H]⁺ ¹H NMR (400 MHz, MeOH-d₄) δ: 1.33 (s, 4H), 1.71-1.75 (m, 2H), 1.93-1.98 (m, 2H), 3.72 (s, 4H), 3.84 (s, 2H), 3.97 (s, 3H), 6.40 (d, 1H), 6.88 (s, 1H), 7.52 (t, 1H), 7.58 (s, 1H), 7.66 (br d, 1H), 8.86 (s, 1 H). |
| 32 | 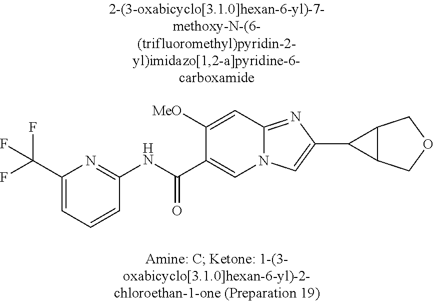 [SMILES: COc1cc2nc(C3C4COCC43)cn2cc1C(=O)Nc1cccc(C(F)(F)F)n1] | White solid (1.5 mg, 0,6%). LCMS m/z = 419 [M + H]⁺ ¹H NMR (400 MHz, MeOH-d₄) δ: 1.80-1.87 (m, 1H), 2.03 (td, 2H), 3.73 (t, 3H), 3.91 (d, 2H), 4.00-4.05 (m, 4H), 6.87 (s, 1H), 7.45-7.51 (m, 2H), 7.97 (t, 2H), 8.49 (d, 1H), 8.91 (s, 1H). |
| 33 | 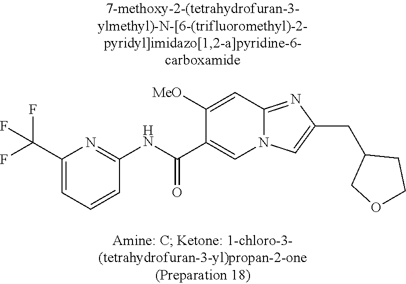 [SMILES: COc1cc2nc(CC3CCOC3)cn2cc1C(=O)Nc1cccc(C(F)(F)F)n1] | White solid (6.9 mg, 7%). LCMS m/z = 421 [M + H]⁺ ¹H NMR (400 MHz, MeOH-d₄) δ: 1.67-1.78 (m, 1H), 2.08-2.16 (m, 1H), 2.66-2.79 (m, 1H), 2.80-2.86 (m, 2H), 3.50-3.58 (m, 1H), 3.74- 3.84 (m, 2H), 3.87-3.95 (m, 3H), 4.12-4.19 (m, 4H), 7.01 (s, 1H), 7.58 (d, 1H), 7.65 (s, 1H), 8.08 (t, 1H), 8.60 (d, 1H), 9.05 (s, 1H). |
| 34 | 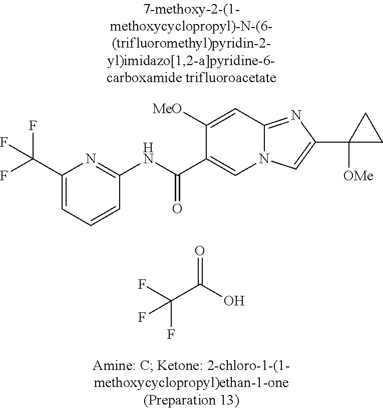 [SMILES: COc1cc2nc(C3(OC)CC3)cn2cc1C(=O)Nc1cccc(C(F)(F)F)n1 ; O=C(O)C(F)(F)F] | White solid (0.5 mg, 1%). LCMS m/z = 407 [M + H]⁺ |
| 35 | 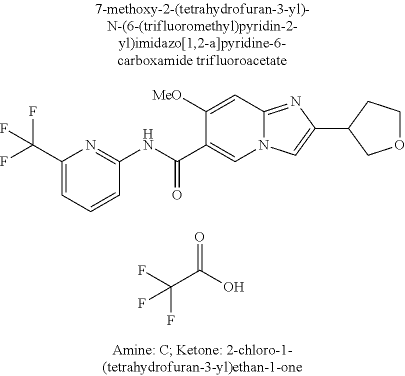 [SMILES: COc1cc2nc(C3CCOC3)cn2cc1C(=O)Nc1cccc(C(F)(F)F)n1 ; O=C(O)C(F)(F)F] | White solid (4.3 mg, 6%). LCMS m/z = 407 [M + H]⁺ ¹H NMR (500 MHz, DMSO-d₆) δ: 2.06-2.11 (m, 1H), 2.37 (dtd, 1H), 3.59-3.73 (m, 1H), 3.77-3.84 (m, 2H), 3.93 (td, 1H), 4.02-4.05 (m, 5H), 7.23 (s, 1H), 7.71 (d, 1H), 7.95-8.03 (m, 1H), 8.19 (t, 1H), 8.41-8.54 (m, 1H), 9.07 (s, 1H), 11.33 (br s, 1H). |
| 36 | 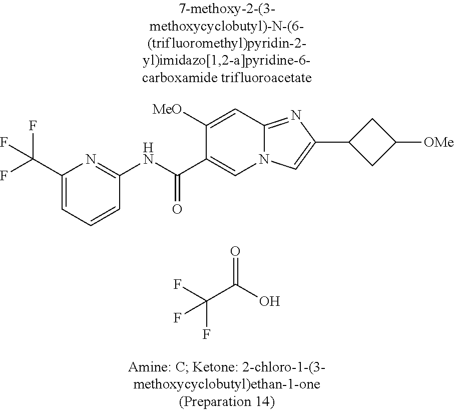 [SMILES: COc1cc2nc(C3CC(OC)C3)cn2cc1C(=O)Nc1cccc(C(F)(F)F)n1 ; O=C(O)C(F)(F)F] | White solid (0.4 mg, 0.4%) LCMS m/z = 421 [M + H]⁺ |
| 37 | 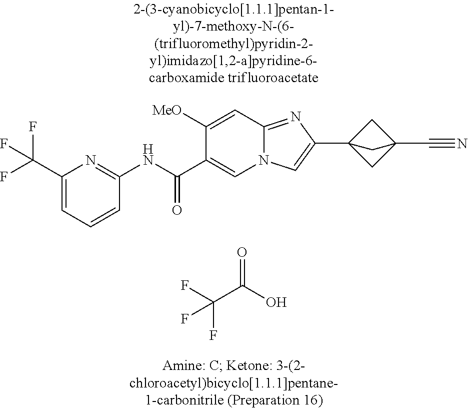 [SMILES: COc1cc2nc(C34CC(C#N)(C3)C4)cn2cc1C(=O)Nc1cccc(C(F)(F)F)n1 ; O=C(O)C(F)(F)F] | White solid (0.6 mg, 0.6%). LCMS m/z = 428 [M + H]⁺ |
| 38 | 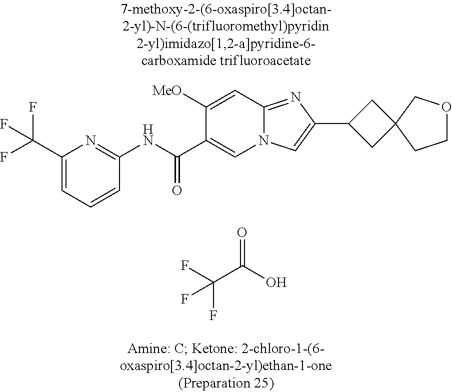 [SMILES: COc1cc2nc(C3CC4(CCOC4)C3)cn2cc1C(=O)Nc1cccc(C(F)(F)F)n1 ; O=C(O)C(F)(F)F] | White solid (0.7 mg, 0.6%). LCMS m/z = 447 [M + H]⁺ |
| 39 | 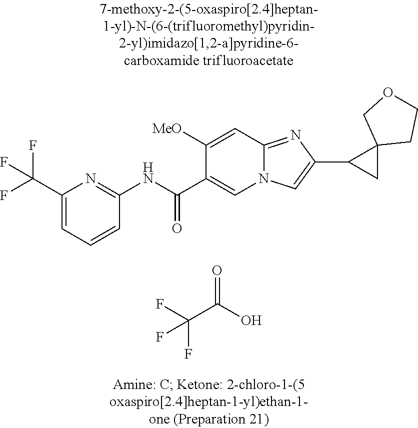 [SMILES: COc1cc2nc(C3CC34CCOC4)cn2cc1C(=O)Nc1cccc(C(F)(F)F)n1 ; O=C(O)C(F)(F)F] | White solid (0.8 mg, 0.7%). LCMS m/z = 433 [M + H]⁺ |
| 40 | 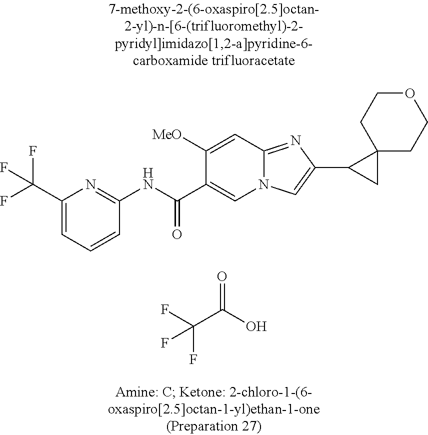 [SMILES: COc1cc2nc(C3CC34CCOCC4)cn2cc1C(=O)Nc1cccc(C(F)(F)F)n1 ; O=C(O)C(F)(F)F] | White solid (0.9 mg, 0.8%). LCMS m/z = 447 [M + H]⁺ |
| 41 | 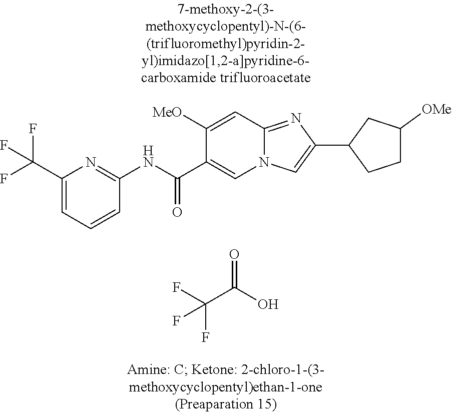 [SMILES: COc1cc2nc(C3CCC(OC)C3)cn2cc1C(=O)Nc1cccc(C(F)(F)F)n1 ; O=C(O)C(F)(F)F] | White solid (0.4 mg, 0.36%). LCMS m/z = 435 [M + H]⁺ |
| 42 | 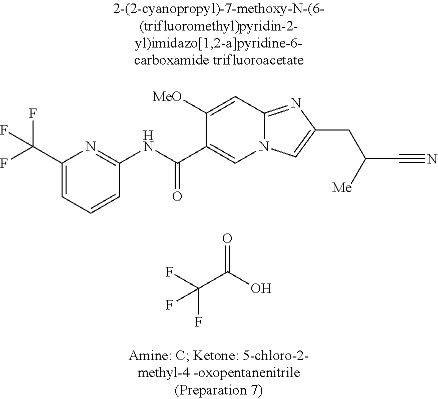 [SMILES: COc1cc2nc(CC(C)C#N)cn2cc1C(=O)Nc1cccc(C(F)(F)F)n1 ; O=C(O)C(F)(F)F] | White solid (1.3 mg, 1.3%). LCMS m/z = 404 [M + H]⁺ |
| 43 | 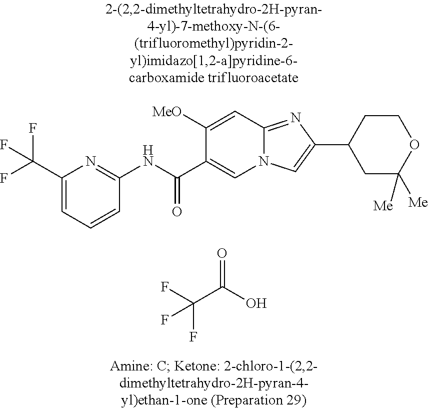 [SMILES: COc1cc2nc(C3CCOC(C)(C)C3)cn2cc1C(=O)Nc1cccc(C(F)(F)F)n1 ; O=C(O)C(F)(F)F] | White solid (1.1 mg, 1%). LCMS m/z = 449 [M + H]⁺ |
| 44 | 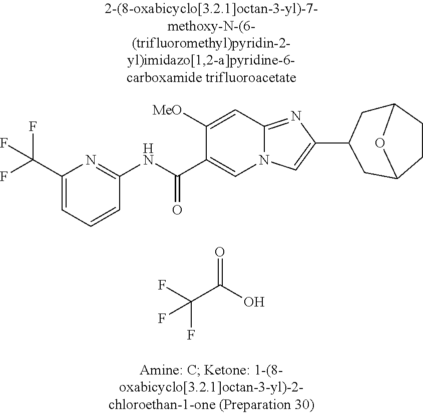 [SMILES: COc1cc2nc(C3CC4CCC(C3)O4)cn2cc1C(=O)Nc1cccc(C(F)(F)F)n1 ; O=C(O)C(F)(F)F] | White solid (0.3 mg, 0.3%). LCMS m/z = 446 [M + H]⁺ |
| 45 | 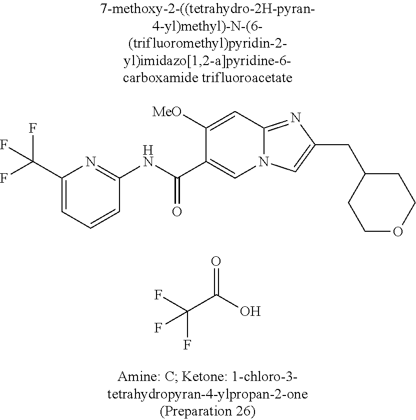 [SMILES: COc1cc2nc(CC3CCOCC3)cn2cc1C(=O)Nc1cccc(C(F)(F)F)n1 ; O=C(O)C(F)(F)F] | White solid (0.3 mg, 0.3%). LCMS m/z = 435 [M + H]⁺ |
| 46 | 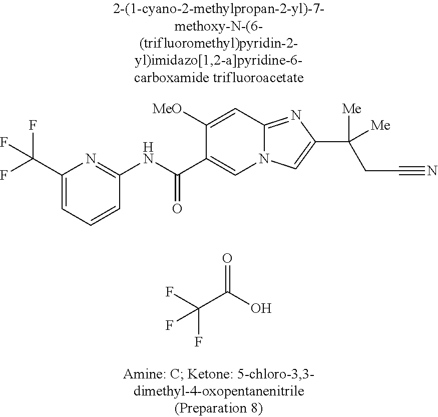 [SMILES: COc1cc2nc(C(C)(C)CC#N)cn2cc1C(=O)Nc1cccc(C(F)(F)F)n1 ; O=C(O)C(F)(F)F] | White solid (0.5 mg, 0.5%). LCMS m/z = 418 [M + H]⁺ |
| 47 | 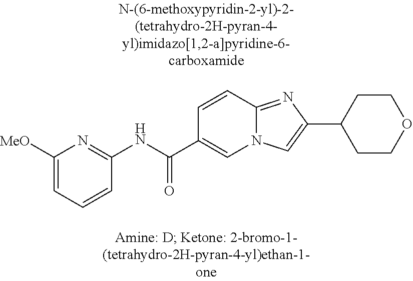 [SMILES: COc1cccc(NC(=O)c2ccc3nc(C4CCOCC4)cn3c2)n1] | White solid (37 mg, 57%). LCMS m/z = 353 [M + H]⁺ ¹H NMR (400 MHz, MeOH-d₄) δ: 1.85-1.98 (m, 2H), 2.06-2.14 (m, 2H), 3.22-3.30 (m, 1H), 3.65 (td, 2H), 3.86-4.00 (m, 3H), 4.11 (dd, 2H), 6.62 (dd, 1H), 7.73 (t, 1H), 7.84 (dd, 1H), 7.93-8.03 (m, 1H), 8.17 (s, 1H), 8.43 (dd, 1H), 9.40 (dd, 1H). |
| 48 | 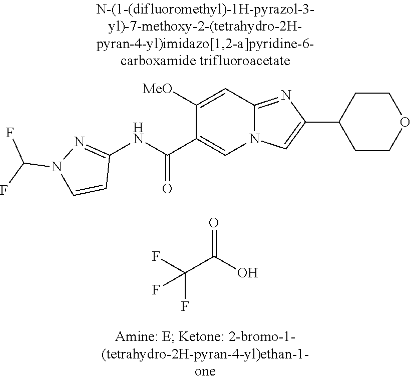 [SMILES: COc1cc2nc(C3CCOCC3)cn2cc1C(=O)Nc1ccn(C(F)F)n1 ; O=C(O)C(F)(F)F] | White solid (15 mg, 27%). LCMS m/z = 392 [M + H]⁺ ¹H NMR (400 MHz, MeOH-d₄) δ: 1.79-1.92 (m, 2H), 2.03-2.10 (m, 3H), 3.13-3.27 (m, 1H), 3.63 (td, 2H), 4.09 (dt, 2H), 4.16-4.26 (m, 3H), 7.01 (d, 1H), 7.24-7.61 (m, 2H), 7.92 (s, 1H), 8.04 (d, 1H), 9.15 (s, 1H). |
| 49 | 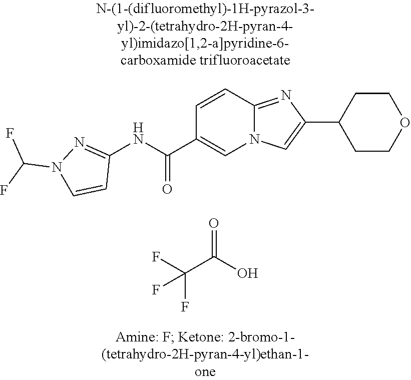 [SMILES: O=C(Nc1ccn(C(F)F)n1)c1ccc2nc(C3CCOCC3)cn2c1 ; O=C(O)C(F)(F)F] | White solid (87.5 mg, 33%). LCMS m/z = 362 [M + H]⁺ ¹H NMR (400 MHz, MeOH-d₄) δ: 1.75-1.92 (m, 2H), 1.96-2.08 (m, 2H), 3.05 (tt, 1H), 3.54-3.65 (m, 2H), 4.05(dt, 2H), 6.96 (d, 1H), 7.25-7.58 (m, 2H), 7.79 (s, 1H), 7.83 (dd, 1H), 7.99 (d, 1H), 9.09(dd, 1H). |

### Examples 50 and 51; chiral SFC: (S)-7-methoxy-N-(6-methoxypyridin-2-yl)-2-((tetrahydrofuran-3-yl)methyl)imidazo[1,2-a]pyridine-6-carboxamide and (R)-7-methoxy-N-(6-methoxypyridin-2-yl)-2-((tetrahydrofuran-3-yl)methyl)imidazo[1,2-a]pyridine-6-carboxamide

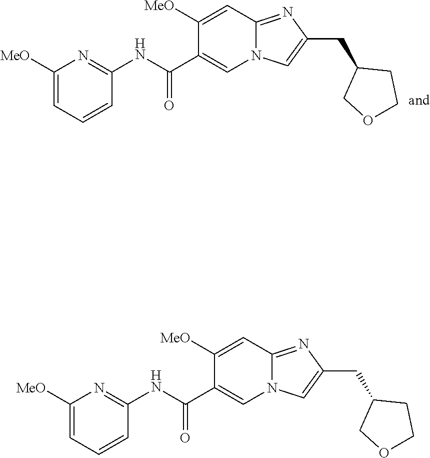 [SMILES: COc1cccc(NC(=O)c2cn3cc(C[C@H]4CCOC4)nc3cc2OC)n1 ; COc1cccc(NC(=O)c2cn3cc(C[C@@H]4CCOC4)nc3cc2OC)n1]

(S)-7-methoxy-N-(6-methoxypyridin-2-yl)-2-((tetrahydrofuran-3-yl)methyl)imidazo[1,2-a]pyridine-6-carboxamide and (R)-7-methoxy-N-(6-methoxypyridin-2-yl)-2-((tetrahydrofuran-3-yl)methyl)imidazo[1,2-a]pyridine-6-carboxamide were obtained from chiral SFC (CHIRALPAK IA 30×250 mm, 5 μm; 40% MeOH w/0.1% DEA in CO₂) purification of Example 24.

Example 50; Peak 1: (S)-7-methoxy-N-(6-methoxypyridin-2-yl)-2-((tetrahydrofuran-3-yl)methyl)imidazo[1,2-a]pyridine-6-carboxamide, 3.0 mg. LCMS m/z=383 [M+H]⁺; ¹H NMR (400 MHz, MeOH-d₄) δ: 1.65-1.79 (m, 1H), 2.05-2.20 (m, 1H), 2.65-2.78 (m, 1H), 2.78-2.86 (m, 2H), 3.54 (dd, 1H), 3.74-3.84 (m, 1H), 3.84-3.97 (m, 5H), 4.14 (s, 3H), 6.58 (d, 1H), 7.00 (s, 1H), 7.57-7.75 (m, 2H), 7.85 (d, 1H), 9.03 (s, 1H).

Example 51; Peak 2: (S)-7-methoxy-N-(6-methoxypyridin-2-yl)-2-((tetrahydrofuran-3-yl)methyl)imidazo[1,2-a]pyridine-6-carboxamide, 3.6 mg. LCMS m/z=383 [M+H]⁺; ¹H NMR (400 MHz, MeOH-d₄) δ: 1.68-1.79 (m, 1H), 2.04-2.19 (m, 1H), 2.67-2.78 (m, 1H), 2.78-2.86 (m, 2H), 3.48-3.60 (m, 1H), 3.75-3.84 (m, 1H), 3.87-3.98 (m, 5H), 4.15 (s, 3H), 6.51-6.63 (m, 1H), 7.00 (s, 1H), 7.57-7.74 (m, 2H), 7.86 (d, 1H), 9.03 (s, 1H).

### Example 52: N-(6-(difluoromethyl)pyridin-2-yl)-8-ethoxy-2-(tetrahydro-2H-pyran-3-yl)imidazo[1,2-a]pyridine-6-carboxamide

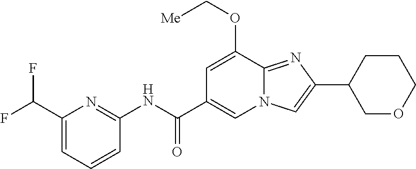 [SMILES: CCOc1cc(C(=O)Nc2cccc(C(F)F)n2)cn2cc(C3CCCOC3)nc12]

T3P® (50 wt. % in EtOAc)® (770 mg, 1.21 mmol, 50% purity) and TEA (203.91 mg, 2.02 mmol) were added to a mixture of 8-ethoxy-2-(tetrahydro-2H-pyran-3-yl)imidazo[1,2-a]pyridine-6-carboxylic acid (Preparation 124, 117 mg, 0.403 mmol) and 6-(difluoromethyl)pyridin-2-amine (116.2 mg, 0.806 mmol) in DMF (1 mL) and stirred at rt for 16 h. The reaction was diluted with water, extracted with EtOAc, dried (Na₂SO₄) and evaporated to dryness in vacuo. The residue was purified by flash chromatography (EtOH/EtOAc; 0-30%) to afford N-(6-(difluoromethyl)pyridin-2-yl)-8-ethoxy-2-(tetrahydro-2H-pyran-3-yl)imidazo[1,2-a]pyridine-6-carboxamide (85 mg, 51%). LCMS 417 [M+H]⁺; ¹H NMR (500 MHz, DMSO-d₆) δ: 1.46 (t, 3H), 1.61-1.69 (m, 2H), 1.74-1.85 (m, 1H), 2.05-2.12 (m, 1H), 2.91-2.99 (m, 1H), 3.37-3.47 (m, 3H), 3.82-3.88 (m, 1H), 3.99-4.04 (m, 1H), 4.29 (q, 2H), 6.90 (t, 1H), 7.18 (d, 1H), 7.48 (d, 1H), 7.84 (s, 1H), 7.83-7.85 (m, 1H), 8.06 (t, 1H), 8.33 (d, 1H), 8.93 (d, 1H), 11.12 (s, 1H),

### Example 53: 8-Ethoxy-N-(6-methoxypyridin-2-yl)-2-(tetrahydro-2H-pyran-3-yl)imidazo[1,2-a]pyridine-6-carboxamide

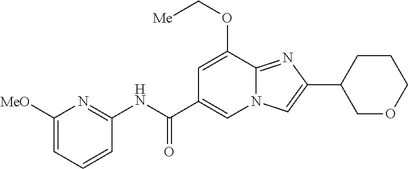 [SMILES: CCOc1cc(C(=O)Nc2cccc(OC)n2)cn2cc(C3CCCOC3)nc12]

The title compound was prepared from 8-ethoxy-2-(tetrahydro-2H-pyran-3-yl)imidazo[1,2-a]pyridine-6-carboxylic acid (Preparation 124) and 6-methoxypyridin-2-amine in an analogous manner to that described for Example 52. The residue after work-up was purified by HPLC (Waters XSelect CSH Prep C18 5 μm OBD 30×50 mm; 5-55% MeCN/H₂O+NH₄OH) to afford 8-ethoxy-N-(6-methoxypyridin-2-yl)-2-(tetrahydro-2H-pyran-3-yl)imidazo[1,2-a]pyridine-6-carboxamideas a colourless glass (2 mg, 1.5%). LCMS=397 [M+H]⁺; ¹H NMR (500 MHz, DMSO-d₆) δ: 1.46 (t, 3H), 1.60-1.71 (m, 2H), 1.74-1.85 (m, 1H), 2.05-2.12 (m, 1H), 2.90-3.00 (m, 1H), 3.39-3.49 (m, 2H), 3.82-3.89 (m, 1H), 3.90 (s, 3H), 3.98-4.06 (m, 1H), 4.28 (q, 2H), 6.60 (dd, 1H), 7.10 (d, 1H), 7.72-7.79 (m, 2H), 7.84 (s, 1H), 8.89 (d, 1H), 10.55 (s, 1H).

### Example 54 and 55: (S)—N-(6-(difluoromethyl)pyridin-2-yl)-8-ethoxy-2-(tetrahydro-2H-pyran-3-yl)imidazo[1,2-a]pyridine-6-carboxamide and (R)—N-(6-(difluoromethyl)pyridin-2-yl)-8-ethoxy-2-(tetrahydro-2H-pyran-3-yl)imidazo[1,2-a]pyridine-6-carboxamide

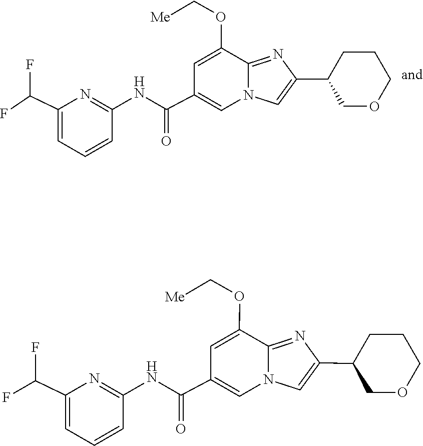 [SMILES: CCOc1cc(C(=O)Nc2cccc(C(F)F)n2)cn2cc([C@@H]3CCCOC3)nc12 ; CCOc1cc(C(=O)Nc2cccc(C(F)F)n2)cn2cc([C@H]3CCCOC3)nc12]

[absolute stereochemistry arbitrarily assigned]

(S)—N-(6-(difluoromethyl)pyridin-2-yl)-8-ethoxy-2-(tetrahydro-2H-pyran-3-yl)imidazo[1,2-a]pyridine-6-carboxamide and (R)—N-(6-(difluoromethyl)pyridin-2-yl)-8-ethoxy-2-(tetrahydro-2H-pyran-3-yl)imidazo[1,2-a]pyridine-6-carboxamide were prepared by SFC separation (CHIRALPAK IB 30×250 mm, 5 μm; 30% EtOH+0.1% DEA in CO₂) of N-(6-(difluoromethyl)pyridin-2-yl)-8-ethoxy-2-(tetrahydro-2H-pyran-3-yl)imidazo[1,2-a]pyridine-6-carboxamide (Example 52).

Peak 1. LCMS=417 [M+H]⁺; ¹H NMR (500 MHz, CDCl₃) δ: 1.45 (t, 3H), 1.61-1.69 (m, 2H), 1.73-1.85 (m, 1H), 2.04-2.11 (m, 1H), 2.52-2.55 (m, 1H), 2.89-2.99 (m, 1H), 3.15-3.19 (m, 1H), 3.40-3.47 (m, 2H), 3.82-3.89 (m, 1H), 3.97-4.05 (m, 1H), 4.29 (q, 2H), 6.93 (t, 1H), 7.18 (d, 1H), 7.47 (d, 1H), 7.84 (s, 1H), 8.05 (t, 1H), 8.33 (d, 1H), 8.93 (d, 1H), 11.12 (s, 1H),

Peak 2. LCMS=417 [M+H]⁺; ¹H NMR (500 MHz, CDCl₃) δ: 1.45 (t, 3H), 1.59-1.71 (m, 2H), 1.72-1.89 (m, 1H), 2.03-2.13 (m, 1H), 2.52-2.55 (m, 1H), 2.90-3.00 (m, 1H), 3.17 (d, 1H), 3.37-3.50 (m, 3H), 3.80-3.90 (m, 1H), 3.97-4.06 (m, 1H), 4.29 (q, 2H), 6.93 (t, 1H), 7.18 (s, 1H), 7.47 (d, 1H), 7.84 (s, 1H), 8.05 (t, 1H), 8.33 (d, 1H), 8.93 (d, 1H), 11.12 (s, 1H).

### Example 56: 8-Methoxy-N-(2-methoxypyridin-3-yl)-2-(1-methyl-2-oxabicyclo[2.1.1]hexan-4-yl)imidazo[1,2-a]pyrazine-6-carboxamide

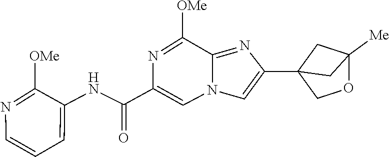 [SMILES: COc1ncccc1NC(=O)c1cn2cc(C34COC(C)(C3)C4)nc2c(OC)n1]

To a mixture of 8-methoxy-2-(1-methyl-2-oxabicyclo[2.1.1]hexan-4-yl)imidazo[1,2-a]pyrazine-6-carboxylic acid (Preparation 132, 15.5 mg, 0.041 mmol) and 2-methoxypyridin-3-amine (5.6 mg, 0.045 mmol) in a microwave reaction vial was added TEA (0.35 mL, 2.52 mmol) followed by T3P® (50 wt. % in EtOAc)® (0.35 mL, >0.77 mmol; 50% purity) and the mixture heated with microwave irradiation at 100° C. for 30 mins. The reaction mixture was quenched by addition of MeOH followed by partitioning between EtOAc and H₂O. The aqueous was extracted (EtOAc) and the combined organics were evaporated to dryness in vacuo. The residue was purified by silica gel chromatography eluting with EtOAc to afford 8-methoxy-N-(2-methoxypyridin-3-yl)-2-(1-methyl-2-oxabicyclo[2.1.1]hexan-4-yl)imidazo[1,2-a]pyrazine-6-carboxamide as an off-white solid (8 mg, 40%). LCMS=396 [M+H]⁺; ¹H NMR (400 MHz, MeOH-d₆) δ: 1.17-1.33 (m, 1H), 1.53 (s, 3H), 1.86-1.96 (m, 2H), 2.13-2.24 (m, 2H), 4.06 (s, 2H), 4.11 (s, 3H), 4.33 (s, 3H), 7.02 (dd, 1H), 7.91 (dd, 1H), 8.03 (s, 1H), 8.71 (dd, 1H), 8.86 (s, 1H).

### Example 57: 7-Methoxy-N-(pyrazolo[1,5-a]pyridin-7-yl)-2-(tetrahydro-2H-pyran-4-yl)imidazo[1,2-a]pyridine-6-carboxamide

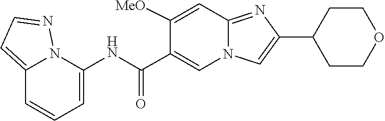 [SMILES: COc1cc2nc(C3CCOCC3)cn2cc1C(=O)Nc1cccc2ccnn12]

A mixture of 7-methoxy-2-(tetrahydro-2H-pyran-4-yl)imidazo[1,2-a]pyridine-6-carboxylic acid (Preparation 75, 50 mg, 0.181 mmol), CDI (29.3 mg, 0.181 mmol) and DMF (1 mL) was stirred at 60° C. for 1 h. Pyrazolo[1,5-a]pyridin-7-amine (22 mg, 0.165 mmol) and tBuONa (47.4 mg, 0.494 mmol) were added under Ar and the vial sealed and stirred at 60° C. for 4 h. The reaction was evaporated to dryness in vacuo and the residue dissolved in DMSO (0.5 mL) and neutralized with AcOH (50 μL) and purified by prep-HPLC (Waters SunFire C18 19×100 5 μm; H₂O/MeOH; Gradient (% organic) 50-100) to afford 7-methoxy-N-(pyrazolo[1,5-a]pyridin-7-yl)-2-(tetrahydro-2H-pyran-4-yl)imidazo[1,2-a]pyridine-6-carboxamide (8 mg, 11.4%). LCMS m/z=392 [M+H]⁺; ¹H NMR (500 MHz, DMSO-d₆) δ: 1.68-1.80 (m, 2H), 1.91-1.97 (m, 2H), 2.86-2.95 (m, 1H), 3.43-3.51 (m, 2H), 3.92-3.98 (m, 2H), 4.23-4.27 (m, 3H), 6.56-6.61 (m, 1H), 7.07-7.11 (m, 1H), 7.21-7.29 (m, 1H), 7.34-7.40 (m, 1H), 7.61-7.66 (m, 1H), 7.82-7.88 (m, 1H), 7.94-8.00 (m, 1H), 9.24-9.29 (m, 1H), 12.01-12.06 (m, 1H).

### Example 58: N-(6-ethylpyridin-2-yl)-2-(1-(fluoromethyl)-2-oxabicyclo[2.1.1]hexan-4-yl)-7-methoxyimidazo[1,2-a]pyridine-6-carboxamide trifluoroacetate

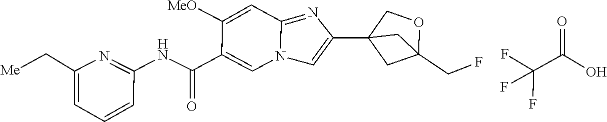

Part A: TFA (327 μL, 4.27 mmol,) was added in a single portion to a vial charged with tert-butyl (5-((6-ethylpyridin-2-yl)carbamoyl)-4-methoxypyridin-2-yl)carbamate (Preparation 86, 159 mg, 0.427 mmol) in DCM (4 mL) at rt. The vial was capped and stirred at rt for 30 min and evaporated to dryness in vacuo to afford 6-amino-N-(6-ethyl-2-pyridyl)-4-methoxy-pyridine-3-carboxamide (400 mg, 98% yield, 6 TFA) which was used in Part B without further purification.

Part B: To a vial charged with the compound of Part A (70 mg, 0.181 mmol, 6 TFA), 2-chloro-1-(1-(fluoromethyl)-2-oxabicyclo[2.1.1]hexan-4-yl)ethan-1-one (Preparation 23, 52.4 mg, 0.272 mmol), and NaHCO₃ (152 mg, 1.81 mmol) was added a 1:1 mixture of PrCN/toluene (2 mL) at rt. The vial was sealed and heated at 100° C. for 18 h, cooled, filtered through a pad of Celite® and evaporated to dryness in vacuo. The residue was purified by prep HPLC (SunFire C18 column, 60 mL/min flow rate, MeCN/H₂O/0.1% TFA; Gradient (% organic): 10-70) to afford N-(6-ethylpyridin-2-yl)-2-(1-(fluoromethyl)-2-oxabicyclo[2.1.1]hexan-4-yl)-7-methoxyimidazo[1,2-a]pyridine-6-carboxamide trifluoroacetate as a white solid (14.5 mg, 15%). LCMS m/z=411 [M+H]⁺; ¹H NMR (500 MHz, DMSO-d₄) δ: 1.15-1.31 (m, 3H), 1.97 (br d, 2H), 2.27-2.33 (m, 2H), 2.71 (q, 2H), 4.00-4.14 (m, 5H), 4.64-4.73 (m, 1H), 4.78 (s, 1H), 7.10 (d, 1H), 7.24 (s, 1H), 7.80 (t, 1H), 7.99-8.18 (m, 2H), 9.11 (s, 1H), 10.79 (br s, 1H).

### Examples 59-62

The title compounds were prepared in an analogous manner to that described for Example 58 using the appropriate halomethyl ketone as shown in the following table:

Purified by prep-HPLC: (SunFire C18 column, 60 mL/min flow rate, MeCN/H₂O/0.1% TFA; Gradient (% organic): 10-70)

| Example | Name/Structure/RCOCH₂Hal | QC Data |
|---|---|---|
| 59 | 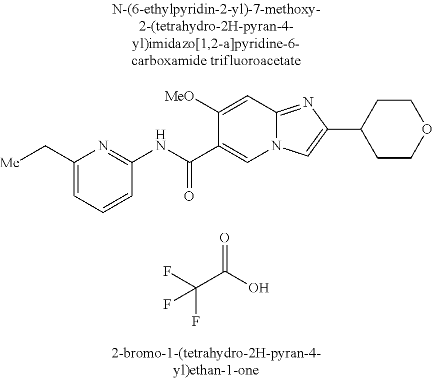 [SMILES: CCc1cccc(NC(=O)c2cn3cc(C4CCOCC4)nc3cc2OC)n1 ; O=C(O)C(F)(F)F] | White solid (6.5 mg, 10%). LCMS m/z = 381 [M + H]⁺ ¹HNMR (500 MHz, DMSO-d₆) δ: 1.23 (br t, 3H), 1.68-1.75 (m, 3H), 1.96 (br dd, 2H), 2.68-2.76 (m, 3H), 3.49 (td, 1H), 3.79-3.89 (m, 2H), 3.95-4.02 (m, 2H), 4.08 (s, 3H), 7.10 (d, 1H), 7.25 (s, 1H), 7.72-7.86 (m, 1H), 7.91-8.10 (m, 2H), 9.11 (s, 1H), 10.75-10.86 (m, 1H). |
| 60 | 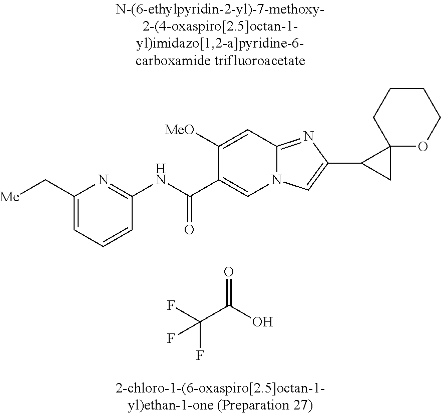 [SMILES: CCc1cccc(NC(=O)c2cn3cc(C4CC45CCCCO5)nc3cc2OC)n1 ; O=C(O)C(F)(F)F] | White solid (2.8 mg, 3%). LCMS m/z = 381 [M + H]⁺ ¹HNMR (500 MHz, DMSO-d₆) δ: 1.03-1.16 (m, 3H), 1.17-1.32 (m, 5H), 1.33- 1.50 (m, 3H), 1.50-1.64 (m, 4H), 2.07 (br t, 1H), 2.14-2.22 (m, 1H), 2.63-2.80 (m, 3H), 3.35 (br s, 1H), 3.46-3.82 (m, 4H), 4.06 (s, 3H), 4.55-4.75 (m, 1H), 7.09 (br d, 1H), 7.22 (s, 1H), 7.79 (t, 1H), 7.89 (s, 1H), 8.03 (br d, 1H), 9.03 (s, 1H), 10.73 (br s, 1H). |
| 61 | 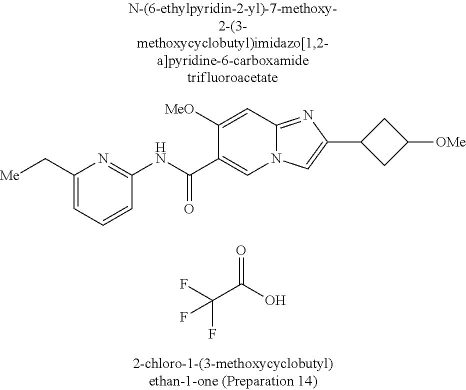 [SMILES: CCc1cccc(NC(=O)c2cn3cc(C4CC(OC)C4)nc3cc2OC)n1 ; O=C(O)C(F)(F)F] | White solid (9.1 mg, 10%). LCMS m/z = 381 [M + H]⁺ ¹HNMR (500 MHz, DMSO-d₆) δ: 1.23 (br t, 3H), 1.99-2.17 (m, 2H), 2.39-2.48 (m, 1H), 2.66-2.78 (m, 4H), 3.17- 3.24 (m, 2H), 3.85-3.98 (m, 1H), 4.05-4.13 (m, 3H), 7.10 (br d, 1H), 7.18-7.30 (m, 1H), 7.80 (t, 1H), 7.93- 8.14 (m, 2H), 9.03-9.15 (m, 1H), 9.12-9.14 (m, 1H), 10.81 (br s, 1H). |
| 62 | 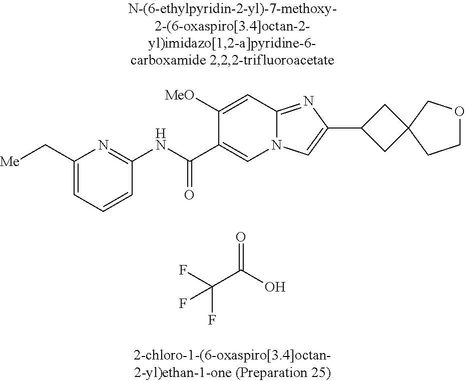 [SMILES: CCc1cccc(NC(=O)c2cn3cc(C4CC5(CCOC5)C4)nc3cc2OC)n1 ; O=C(O)C(F)(F)F] | White solid (4 mg, 4%). LCMS m/z = 407 [M + H]⁺ ¹H NMR (500 MHz, DMSO-d₆) δ: 1.23 (br t, 3H), 1.90- 1.99 (m, 1H), 2.08 (t, 1H), 2.28-2.36 (m, 2H), 2.37-2.48 (m, 2H), 2.67- 2.79 (m, 3H), 3.58-3.63 (m, 1H), 3.66-3.80 (m, 4H), 4.02-4.10 (m, 3H), 7.10 (d, 1H), 7.23 (s, 1H), 7.72- 7.86 (m, 1H), 7.94-8.10 (m, 2H), 9.08 (s, 1H), 10.70-10.83 (m, 1H). |

### Example 63: 8-(Difluoromethoxy)-N-(6-(difluoromethyl)pyridin-2-yl)-2-(tetrahydro-2H-pyran-4-yl)imidazo[1,2-a]pyridine-6-carboxamide trifluoroacetate

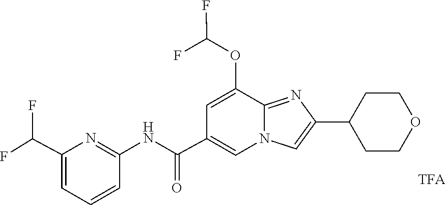 [SMILES: O=C(Nc1cccc(C(F)F)n1)c1cc(OC(F)F)c2nc(C3CCOCC3)cn2c1]

A mixture of 6-amino-5-(difluoromethoxy)-N-(6-(difluoromethyl)pyridin-2-yl)nicotinamide (Preparation 138, 100 mg, 0.302 mmol), 2-bromo-1-tetrahydropyran-4-yl-ethan-1-one (37 mg, 0.181 mmol) and NaHCO₃ (76 mg, 0.908 mmol) in MeCN (0.8 mL) and toluene (0.5 mL) was heated at 90° C. for 16 h. After the addition of silica and MeOH, the mixture was concentrated and purified by reverse-phase HPLC (Waters SunFire Prep C18 5 μm OBD 19×100 mm; MeCN/H₂O+0.1% TFA: gradient (% organic) 5-95) to provide 8-(difluoromethoxy)-N-(6-(difluoromethyl)pyridin-2-yl)-2-(tetrahydro-2H-pyran-4-yl)imidazo[1,2-a]pyridine-6-carboxamide trifluoroacetate (29.9 mg, 2%). LCMS m/z=439 [M+H]⁺; ¹H NMR (500 MHz, DMSO-d₆) δ: 1.68-1.78 (m, 2H), 1.96 (br dd, 2H), 3.01 (tt, 1H), 3.49 (td, 2H), 3.95 (dt, 2H), 6.82-7.07 (m, 1H), 7.47-7.67 (m, 3H), 7.97 (s, 1H), 8.08 (t, 1H), 8.34 (s, 1H), 9.23 (d, 1H), 11.24 (s, 1H).

### Example 64 and Example 65: N-[6-(difluoromethyl)-2-pyridyl]-7-ethoxy-2-[[(3S)-tetrahydrofuran-3-yl]methyl]imidazo[1,2-a]pyridine-6-carboxamide and N-[6-(difluoromethyl)-2-pyridyl]-7-ethoxy-2-[[(3R)-tetrahydrofuran-3-yl]methyl]imidazo[1,2-a]pyridine-6-carboxamide

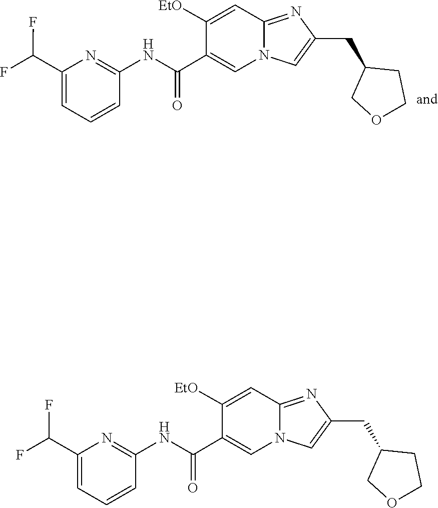 [SMILES: CCOc1cc2nc(C[C@H]3CCOC3)cn2cc1C(=O)Nc1cccc(C(F)F)n1 ; CCOc1cc2nc(C[C@@H]3CCOC3)cn2cc1C(=O)Nc1cccc(C(F)F)n1]

Part 1.

To a solution of methyl 7-ethoxy-2-((tetrahydrofuran-3-yl)methyl)imidazo[1,2-a]pyridine-6-carboxylate (Preparation 64, 170 mg, 0.559 mmol) in MeOH (10 mL) and water (3 mL) was added NaOH (67 mg, 1.68 mmol). The mixture was stirred at 10-15° C. for 12 h. MeOH was removed under reduced pressure and the aqueous layer acidified to pH 3 with aq. HCl (1 M). The mixture was lyophilized to give 7-ethoxy-2-((tetrahydrofuran-3-yl)methyl)imidazo[1,2-a]pyridine-6-carboxylic acid as a yellow solid which was used without further purification in the Part 2

Part 2.

A solution of 7-ethoxy-2-((tetrahydrofuran-3-yl)methyl)imidazo[1,2-a]pyridine-6-carboxylic acid (Part A) and 6-(difluoromethyl)pyridin-2-amine (130 mg, 0.899 mmol) in T3P® (50 wt. % in EtOAc)® (2 mL, 50% in EtOAc) and TEA (14.4 mmol, 2.00 mL) was stirred at 85° C. for 1 h. The mixture was diluted with saturated aq. NaHCO₃ (50 mL) and extracted with EtOAc (3×50 mL). The combined extracts were washed with brine (100 mL), dried (Na₂SO₄) and evaporated to dryness in vacuo and the residue purified by prep-HPLC (Phenomenex Synergi C18 150×30 mm×4 μm, MeCN/H₂O+0.225% HCO₂H; gradient (% organic): 0-100; gradient shape optimized for individual separations). The residue was purified by chiral-SFC chromatography (Diacel Chiralpak AD-H; 250 mm×30 mm×5 μm; 0.1% NH₄OH in EtOH; Flow Rate: 60 mL/min) to afford N-[6-(difluoromethyl)-2-pyridyl]-7-ethoxy-2-[[(3S)-tetrahydrofuran-3-yl]methyl]imidazo[1,2-a]pyridine-6-carboxamide and N-[6-(difluoromethyl)-2-pyridyl]-7-ethoxy-2-[[(3R)-tetrahydrofuran-3-yl]methyl]imidazo[1,2-a]pyridine-6-carboxamide:

Peak 1: Example 64, N-[6-(difluoromethyl)-2-pyridyl]-7-ethoxy-2-[[(3S)-tetrahydrofuran-3-yl]methyl]imidazo[1,2-a]pyridine-6-carboxamide as a white solid (24 mg, 10%). LCMS m/z=417 [M+H]⁺; ¹H NMR (500 MHz, MeOH-d₄) δ: 1.65 (t, 3H), 1.35-1.40 (m, 1H), 2.10-2.20 (m, 1H), 2.60-2.70 (m, 1H), 2.75-2.80 (m, 2H), 3.50-3.55 (m, 1H), 3.70-3.78 (m, 1H), 3.80-3.90 (m, 2H), 4.36 (q, 2H), 6.50-6.80 (m, 2H), 6.72 (s, 1H), 7.44 (d, 1H) 7.64 (s, 1H), 7.99 (t, 1H), 8.45 (d, 1H), 9.08 (s, 1H).

and

Peak 2: Example 65, N-[6-(difluoromethyl)-2-pyridyl]-7-ethoxy-2-[[(3R)-tetrahydrofuran-3-yl]methyl]imidazo[1,2-a]pyridine-6-carboxamide as a white solid (27 mg, 11%). LCMS m/z=417 [M+H]⁺; ¹H NMR (500 MHz, MeOH-d₄) δ: 1.65 (d, 3H), 1.35-1.45 (m, 1H), 2.10-2.20 (m, 1H), 2.60-2.70 (m, 1H), 2.75-2.80 (m, 2H), 3.50-3.55 (m, 1H), 3.70-3.78 (m, 1H), 3.80-3.90 (m, 2H), 4.36 (q, 2H), 6.50-6.80 (m, 2H), 6.72 (s, 1H), 7.44 (d, 1H), 7.64 (s, 1H), 7.99 (t, 1H), 8.45 (d, 1H), 9.08 (s, 1H).

### Examples 66-71

The title compounds were prepared in an analogous method to Example 65 using the appropriate ester and the appropriate amine and preparative SFC using the conditions shown in the following table.

| Example | SFC Conditions/Name/Yield/Structure | Ester, Amine, Data |
|---|---|---|
| 66 and 67 | 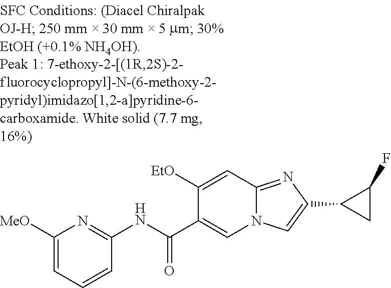 [SMILES: CCOc1cc2nc([C@H]3C[C@@H]3F)cn2cc1C(=O)Nc1cccc(OC)n1] | Ester: Rac-methyl 7-ethoxy-2- ((1S,2R)-2- fluorocyclopropyl)imidazo[1,2- a]pyridine-6-carboxylate (Preparation 54), Amine: 6- methoxypyridin-2-amine Peak 1: LCMS m/z = 371 [M + H]⁺ ¹H NMR (500 MHz, DMSO-d₆) δ: 1.19-1.23 (m, 2H), 1.50-1.57 (m, 3H), 2.56-2.59 (m, 1H), 3.83 (s, 3H), 4.26-4.30 (m, 2H), 4.84-4.99 (m, 1H), 6.60 (d, 1H), 7.05 (s, 1H), 7.74-7.82 (m, 3H), 9.12 (s, 1H), 10.54 (s, 1H). |
| | 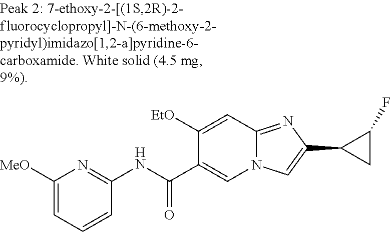 [SMILES: CCOc1cc2nc([C@@H]3C[C@H]3F)cn2cc1C(=O)Nc1cccc(OC)n1] | Peak 2: LCMS m/z = 371 [M + H]⁺ ¹H NMR (500 MHz, DMSO-d₆) δ: 1.19-1.23 (m, 2H), 1.50-1.57 (m, 3H), 2.56-2.59 (m, 1H), 3.83 (s, 3H), 4.26-4.30 (m, 2H), 4.84-4.98 (m, 1H), 6.60 (d, 1H), 7.05 (s, 1H), 7.74-7.82 (m, 3H), 9.12 (s, 1H), 10.54 (s, 1H). |
| 68 And 69 | 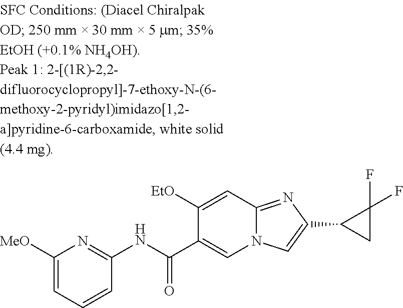 [SMILES: CCOc1cc2nc([C@H]3CC3(F)F)cn2cc1C(=O)Nc1cccc(OC)n1] | Ester: methyl 2-(2,2- difluorocyclopropyl)-7- ethoxyimidazo[1,2-a]pyridine-6- carboxylate (Preparation 51) Amine: 6-methoxypyridin-2-amine Peak 1: LCMS m/z = 389 [M + H]⁺ ¹H NMR (500 MHz, DMSO-d₆) δ: 1.56 (t, 3H), 1.93-2.04 (m, 2H), 3.01-3.09 (m, 1H), 3.83 (s, 3H), 4.27-4.32 (m, 2H), 6.60 (d, 1H), 7.11 (s, 1H), 7.75-7.82 (m, 2H), 7.89 (m, 1H), 9.14 (s, 1H), 10.56 (s, 1H). |
| | 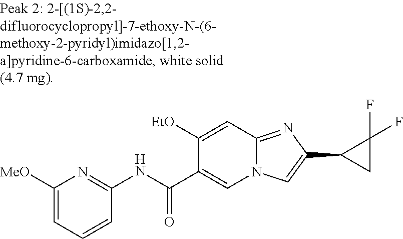 [SMILES: CCOc1cc2nc([C@@H]3CC3(F)F)cn2cc1C(=O)Nc1cccc(OC)n1] | Peak 2. LCMS m/z = 389 [M + H]⁺ ¹H NMR (500 MHz, DMSO-d₆) δ: 1.56 (t, 3H), 1.93-2.04 (m, 2H), 3.01-3.09 (m, 1H), 3.83 (s, 3H), 4.27-4.32 (m, 2H), 6.60 (d, 1H), 7.10 (s, 1H), 7.74-7.82 (m, 2H), 7.89 (m, 1H), 9.14 (s, 1H), 10.56 (s, 1H). |
| 70 And 71 | 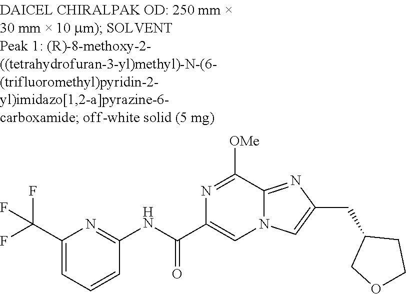 [SMILES: COc1nc(C(=O)Nc2cccc(C(F)(F)F)n2)cn2cc(C[C@@H]3CCOC3)nc12] | Ester: methyl 8-methoxy-2- ((tetrahydrofuran-3- yl)methyl)imidazo[1,2-a]pyrazine- 6-carboxylate (Preparation 71) Amine: 2-amino-6- (trifluoromethyl)pyridine Peak 1. LCMS m/z = 422 [M + H]⁺ ¹H NMR (400 MHz, MeOH-d₄) δ: 1.66-1.75 (m, 1H), 2.03-2.14 (m, 1H), 2.87-2.79 (m, 1H), 2.89 (d, 2H), 3.50-3.54 (m, 1H), 3.74-3.81 (m, 1H), 3.85-3.94 (m, 2H), 4.40 (s, 3H), 7.60 (d, 1H), 7.95 (s, 1H), 8.10 (t, 1H), 8.64 (d, 1H), 8.92 (s, 1H). |
| | 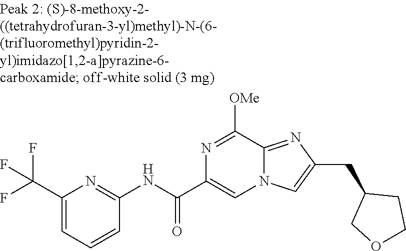 [SMILES: COc1nc(C(=O)Nc2cccc(C(F)(F)F)n2)cn2cc(C[C@H]3CCOC3)nc12] | Peak 2. LCMS m/z = 422 [M + H]⁺ ¹H NMR (400 MHz, MeOH-d₄) δ: 1.66-1.75 (m, 1H), 2.05-2.14 (m, 1H), 2.67-2.78 (m, 1H), 2.89 (d, 2H), 3.49-3.54 (m, 1H), 3.74-3.81 (m, 1H), 3.85-3.93 (m, 2H), 4.34 (s, 3H), 7.59 (d, 1H), 7.94 (s, 1H), 8.09 (t, 1H), 8.64 (d, 1H), 8.91 (s, 1H). |

### Examples 72-85

The title compounds were prepared in an analogous method to Example 66 using the appropriate esters and amines and preparative HPLC without additional SFC separation using the conditions shown in the following table.

| Example | Structure/Name/Reactants/HPLC Conditions | Yield/Data |
|---|---|---|
| 72 | 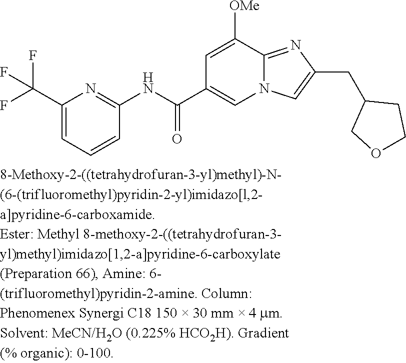 [SMILES: COc1cc(C(=O)Nc2cccc(C(F)(F)F)n2)cn2cc(CC3CCOC3)nc12] | White solid (6 mg, 8%) LCMS m/z = 421 [M + H]⁺ ¹H NMR (500 MHz, MeOH-d₄) δ: 1.68-1.74 (m, 1H), 2.07-2.16 (m, 1H), 2.73-2.76 (m, 1H), 2.91 (d, 2H), 3.53-3.54 (m, 1H), 3.78-3.80 (m, 1H), 3.87-3.93 (m, 2H), 4.17 (s, 3H), 7.50 (s, 1H), 7.59 (d, 1H), 7.94 (s, 1H), 8.08 (t, 1H), 8.54 (d, 1H), 8.87 (s, 1H). |
| 73 | 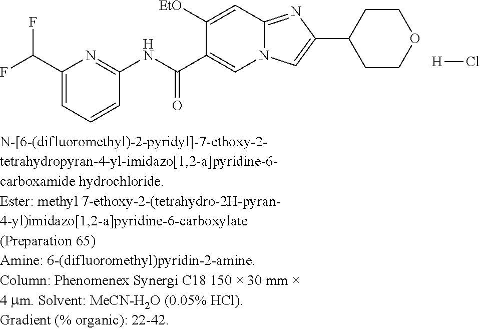 | Brown solid (9.7 mg, 22%). LCMS m/z = 417 [M + H]⁺ ¹H NMR (400 MHz, MeOH-d₄) δ: 1.63 (t, 3H), 1.80-1.91 (m, 2H), 2.02-2.06 (m, 2H), 3.15-3.22 (m, 1H), 3.58- 3.65 (m, 2H), 4.05-4.10 (m, 2H), 4.44-4.50 (m, 2H), 6.48-6.77 (m, 1H), 7.29 (s, 1H), 7.48 (d, 1H), 7.92 (s, 1H), 8.00- 8.05 (m, 1H), 8.44 (d, 1H), 9.20 (s, 1H). |
| 74 | 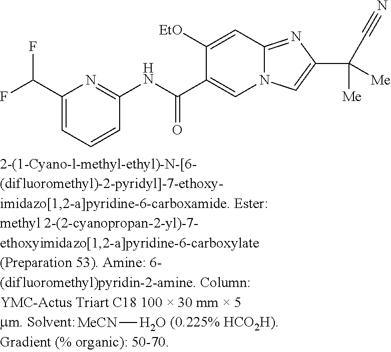 | Brown solid (2.5 mg, 4%). LCMS m/z = 400 [M + H]⁺ ¹H NMR (500 MHz, MeOH-d₄) δ: 1.65 (t, 1H), 1.80 (s, 6H), 4.36- 4.41 (m, 2H), 6.50-6.73 (m, 1H), 7.02 (s, 1H), 7.45 (d, 1H), 7.86 (s, 1H), 7.98-8.02 (m, 1H), 8.44 (d, 1H), 9.11 (s, 1H), |
| 75 | 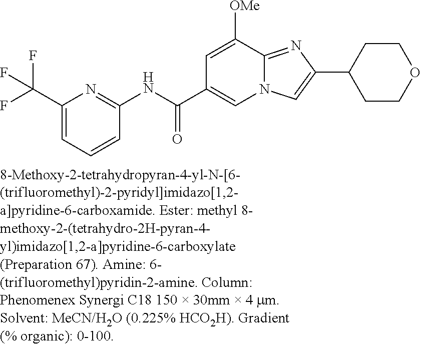 [SMILES: COc1cc(C(=O)Nc2cccc(C(F)(F)F)n2)cn2cc(C3CCOCC3)nc12] | Brown solid (90 mg). LCMS m/z = 421 [M + H]⁺ ¹H NMR (500 MHz, MeOH-d₄) δ: 1.83-1.88 (m, 2H), 2.03-2.07 (m, 2H), 3.04-3.11 (m, 1H), 3.60-3.65 (m, 2H), 4.05- 4.09 (m, 2H), 4.14 (s, 3H), 7.31 (s, 1H), 7.59 (d, 1H), 7.81 (s, 1H), 8.06-8.12 (m, 1H), 8.55 (d, 1H), 8.82 (d, 1H). |
| 76 | 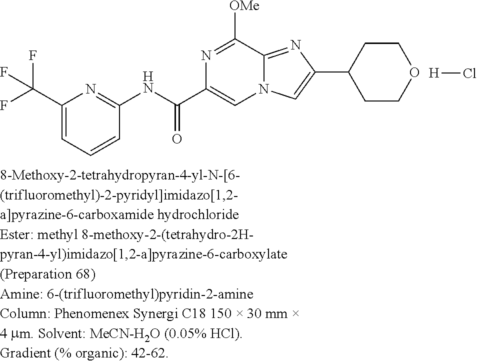 | White solid (15 mg, 20%). LCMS m/z = 422 [M + H]⁺ 1H NMR (500 MHz, MeOH-d₄) δ: 1.86-1.91 (m, 2H), 2.03-2.06 (m, 2H), 3.19-3.31 (m, 1H), 3.59-3.64 (m, 2H), 4.04- 4.09 (m, 2H), 4.44 (s, 3H), 7.63 (d, 1H), 8.13 (t, 1H), 8.19 (s, 1H), 8.66 (d, 1H), 9.08 (s, 1H). |
| 77 | 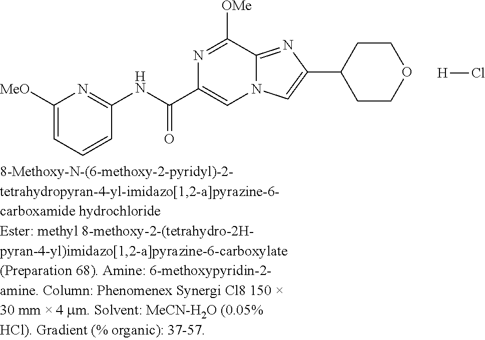 | Yellow solid (15 mg, 22%). LCMS m/z = 384 [M + H]⁺ ¹H NMR (400 MHz, MeOH-d₄) δ: 1.88-1.93 (m, 2H), 2.04-2.08 (m, 2H), 3.22-3.32 (m, 1H), 3.59-3.65 (m, 2H), 3.93 (s, 3H), 4.06-4.11 (m, 2H), 4.46 (s, 3H), 6.63 (d, 1H), 7.76 (t, 1H), 7.90 (d, 1H), 8.30 (s, 1H), 9.10 (s, 1H). |
| 78 | 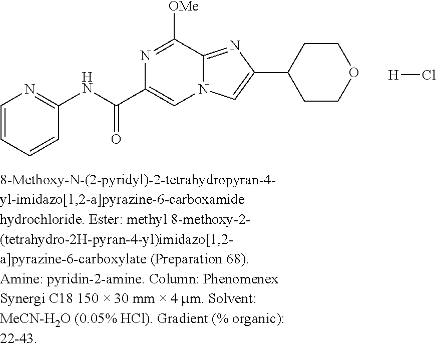 | Yield: 5 mg, 8% LCMS m/z = 353 [M + H]⁺ ¹H NMR (400 MHz, MeOH-d₄) δ: 1.88-1.92 (m, 2H), 2.05-2.08 (m, 2H), 3.24-3.32 (m, 1H), 3.60-3.65 (m, 2H), 4.06- 4.10 (m, 2H), 4.51 (s, 3H), 7.71 (t, 1H), 8.33 (s, 1H), 8.35 (d, 1H), 8.50- 8.57 (m, 1H), 8.57 (d, 1H), 9.26 (s, 1H). |
| 79 | 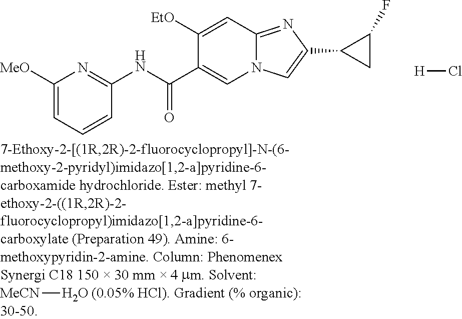 | White solid (35.5 mg, 63%). LCMS m/z = 371 [M + H]⁺ ¹H NMR (500 MHz, DMSO-d6) δ: 1.45- 1.49 (m, 1H), 1.48-1.53 (m, 3H), 1.53-1.60 (m, 1H), 2.36-2.42 (m, 1H), 3.84 (s, 3H), 4.39 (d, 2H), 5.07-5.22 (m, 1H), 6.62-6.65 (m, 1H), 7.28 (s, 1H), 7.79 (d, 2H), 8.04 (s, 1H), 9.25 (s, 1H), 10.61 (s, 1H). |
| 80 | 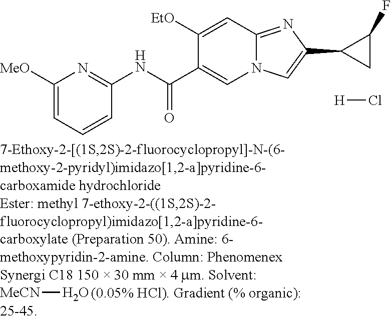 | White solid (23.2 mg, 55%). LCMS m/z = 371 [M + H]⁺ ¹H NMR (400 MHz, DMSO-d₆) δ: 1.44- 1.48 (m, 1H), 1.48-1.53 (m, 3H), 1.54-1.59 (m, 1H), 2.37-2.42 (m, 1H), 3.87 (s, 3H), 4.40 (d, 2H), 5.08-5.24 (m, 1H), 6.62-6.65 (m, 1H), 7.28 (s, 1H), 7.79 (d, 2H), 8.05 (s, 1H), 9.25 (s, 1H), 10.62 (s, 1H). |
| 81 | 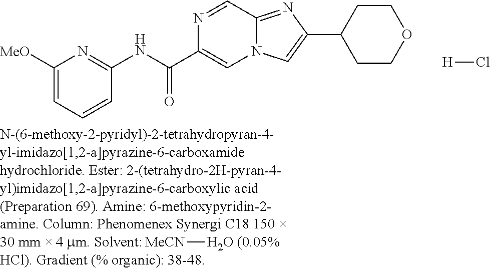 | Yellow solid (1.90 mg, 1.3%). LCMS m/z = 354 [M + H]⁺ ¹H NMR (500 MHz, MeOH-d₄) δ: 2.12 (dd, 2H), 3.38-3.41 (m, 1H), 3.64-3.70 (m, 2H), 3.97 (s, 3H),4.12(dd, 2H), 6.67 (d, 1H), 7.79 (t, 1H), 7.94 (d, 1H), 8.44 (s, 1H), ), 9.42 (s, 1H), 9.57 (d, 1H). |
| 82 | 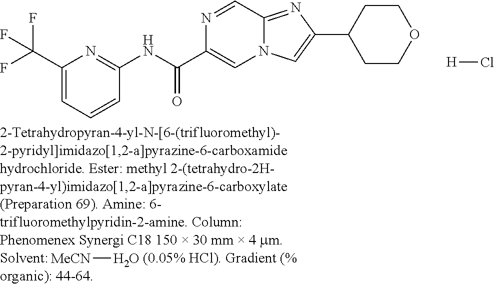 | Yellow solid (1.80 mg, 2.3%). LCMS m/z = 392 [M + H]⁺ ¹H NMR (500 MHz, MeOH-d₄) δ: 1.90- 1.99 (m, 2H), 2.09-2.12 (m, 2H), 3.31 (d, 1H), 3.66 (dd, 2H), 4.41 (dd, 2H), 7.65 (d, 1H), 8.15 (t, 2H), 8.34 (s, 1H), 8.67 (d, 1H), 9.32 (s, 1H), 9.53 (s, 1H), |
| 83 | 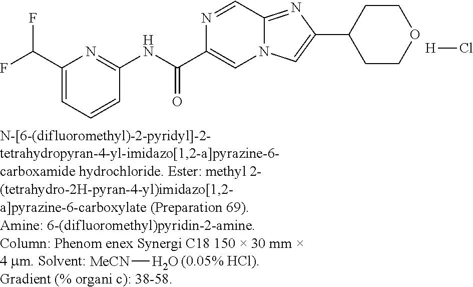 | Yellow solid (3.1 mg, 2%). LCMS m/z = 374 [M + H]^{+ 1}H NMR (500 MHz, MeOH-d₄) δ: 1.91- 2.00 (m, 2H), 2.13 (dd, 2H), 3.4 (tt, 1H), 3.67 (td, 2H), 4.12 (dd, 2H), 6.61-6.84 (m, 1H), 7.54 (d, 1H), 8.47 (s, 1H), 8.65 (d, 1H), 9.46 (s, 1H), 9.62 (d, 1H). |
| 84 | 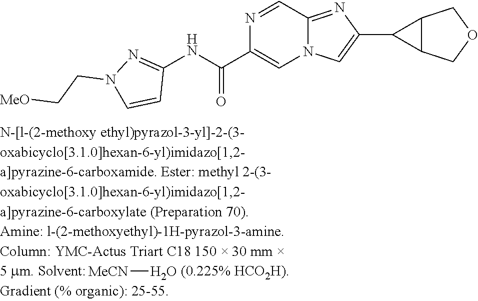 | White solid (1.2 mg, 2.7%). LCMS m/z = 391 [M + Na]^{+ 1}H NMR (400 MHz, MeOH-d₄) δ: 2.07 (t, 1H), 2.22-2.23 (m, 2H), 3.33 (s, 3H), 3.75 (t, 2H), 3.83 (d, 2H), 4.02 (d, 2H), 4.25 (t, 2H), 6.71 (d, 1H), 7.59 (d, 1H), 8.01 (s, 1H), 8.90 (d, 1H), 9.17 (d, 1H). |
| 85 | 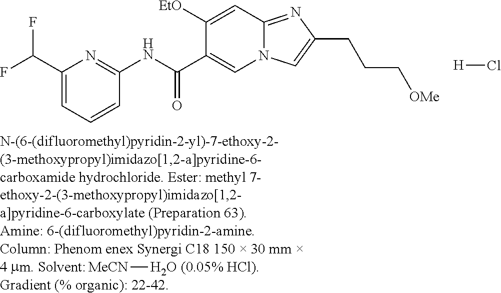 | White solid (3.6 mg, 8.2%). LCMS m/z = 405 [M + H]^{+ 1}H NMR (400 MHz, MeOH-d₄) δ: 1.63 (t, 3H), 1.99-2.05 (m, 2H), 2.92 (t, 2H), 3.35 (s, 3H), 3.50 (t, 2H), 4.42- 4.49 (m, 2H), 6.49-6.76 (m, 1H), 7.27 (s, 1H), 7.47 (d, 1H), 7.86 (s, 1H), 7.99-8.04 (m, 1H), 8.43 (d, 1H), 9.19 (s, 1H). |
| 86 | 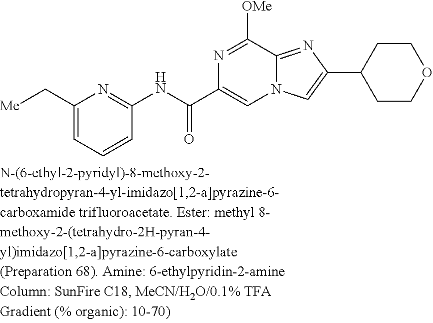 [SMILES: CCc1cccc(NC(=O)c2cn3cc(C4CCOCC4)nc3c(OC)n2)n1] | White solid (11.9 mg, 12%). LCMS m/z = 404 [M + H]⁺ ¹H NMR (500 MHz, CDCl) δ: 1.44 (t, 3H), 1.87 (qd, 2H), 2.10 (br dd, 2H), 3.08, (q, 2H), 3.25 (tt, 1H), 3.64 (td, 2H), 4.15 (dd, 2H), 4.26-4.36 (m, 3H), 7.33 (d, 1H), 7.59 (s, 1H), 8.25 (dd, 1H), 8.71 (d, 1H), 8.74 (s, 1H). |
| 87 | 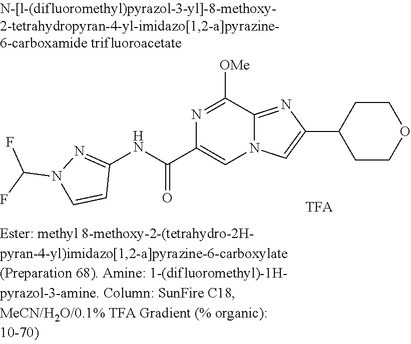 [SMILES: COc1nc(C(=O)Nc2ccn(C(F)F)n2)cn2cc(C3CCOCC3)nc12] | White solid (11 mg, 11%). LCMS m/z = 393 [M + H]⁺ ¹H NMR (500 MHz, MeOH-d₄) δ: 1.81- 1.96 (m, 2H), 1.98-2.11 (m, 3H), 3.13-3.23 (m, 1H), 3.63 (td, 2H), 4.08 (br dd, 2H), 4.34-4.44 (m, 3H), 7.05 (d, 1H), 7.31-7.59 (m, 1H), 8.06 (d, 1H), 8.13 (s, 1H), 8.95-9.02 (m, 1H). |
| 88 | 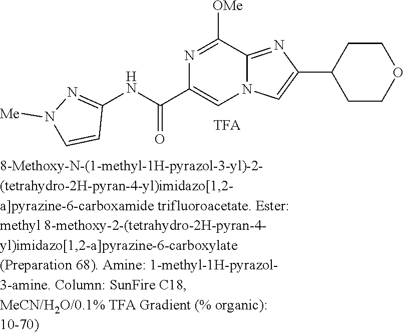 [SMILES: COc1nc(C(=O)Nc2ccn(C)n2)cn2cc(C3CCOCC3)nc12] | White solid (13 mg, 15%). LCMS m/z = 357 [M + H]^{+ 1}H NMR (400 MHz, MeOH-d₄) δ: 1.79- 1.98 (m, 2H), 2.01-2.09 (m, 3H), 3.19 (tt, 1H), 3.62 (td, 11.80, 2H), 3.87 (s, 3H), 4.03-4.15 (m, 2H), 4.39 (s, 3H), 6.71 (d, 1H), 7.56 (d, 1H), 8.14 (s, 1H), 8.96 (s, 1H). |
| 89 | 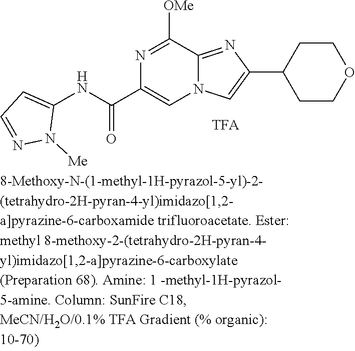 [SMILES: COc1nc(C(=O)Nc2ccnn2C)cn2cc(C3CCOCC3)nc12] | White solid (17 mg, 17%). ¹H NMR (400 MHz, MeOH-d₄) δ: 1.81-1.97 (m, 2H), 2.02-2.11 (m, 2H), 3.19 (tt, 1H), 3.63 (td, 2H), 3.84 (s, 3H), 4.04-4.12 (m, 2H), 4.38 (s, 3H), 6.41 (d, 1H), 7.53 (d, 1H), 8.13 (d, 1H), 8.99 (s, 1H). |

### Example 90: 241-(2,2-Difluoroethyl)azetidin-3-yl]-N-[6-(difluoromethyl)-2-pyridyl]-7-ethoxy-imidazo[1,2-a]pyridine-6-carboxamide

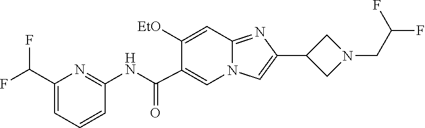 [SMILES: CCOc1cc2nc(C3CN(CC(F)F)C3)cn2cc1C(=O)Nc1cccc(C(F)F)n1]

1,1-difluoro-2-iodoethane (19.8 mg, 0.103 mmol) was added to a solution of 2-(azetidin-3-yl)-N-(6-(difluoromethyl)pyridin-2-yl)-7-ethoxyimidazo[1,2-a]pyridine-6-carboxamide (Preparation 100, 20 mg, 0.051 mmol) and K₂CO₃ (14.3 mg, 0.103 mmol) in MeCN (1 mL) at 15° C. and the mixture stirred at 80° C. for 1 h. The solids were removed by filtration and the filtrate evaporated to dryness in vacuo. The residue was purified by prep-TLC (DCM/MeOH=10/1) and the resulting residue purified by prep-HPLC (Column: Welch Xtimate C18 150×30 mm×5 μm; Solvent:MeCN—H₂O (+10 mM NH₄HCO₃); Gradient (% organic) 35-65) to afford 241-(2,2-difluoroethyl)azetidin-3-yl]-N-[6-(difluoromethyl)-2-pyridyl]-7-ethoxy-imidazo[1,2-a]pyridine-6-carboxamide as a white solid (4.4 mg, 19%). LCMS m/z=474 [M+Na]⁺; ¹H NMR (400 MHz, MeOH-d₄) δ: 1.65 (t, 3H), 2.92-3.02 (m, 2H), 3.48-3.56 (m, 2H), 3.82-3.92 (m, 3H), 4.34-4.40 (m, 2H), 5.72-6.02 (m, 1H), 6.46-6.75 (m, 1H), 6.97 (s, 1H), 7.43 (d, 1H), 7.72 (s, 1H), 7.96-8.01 (m, 1H), 8.43 (d, 1H), 9.07 (s, 1H).

### Example 91: N-(6-(difluoromethyl)pyridin-2-yl)-8-methoxy-2-(1-methoxycyclopropyl)imidazo[1,2-a]pyridine-6-carboxamide

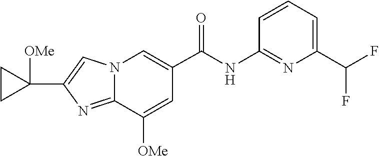 [SMILES: COc1cc(C(=O)Nc2cccc(C(F)F)n2)cn2cc(C3(OC)CC3)nc12]

n-BuLi (0.652 mL, 2.5M in hexane) was added to 6-(difluoromethyl)pyridine-2-amine (235 mg, 1.63 mmol) in THF (15 mL) at −80° C. The mixture was allowed to warm to −30° C., methyl 8-methoxy-2-(1-methoxycyclopropyl)imidazo[1,2-a]pyridine-6-carboxylate (Preparation 62, 187.5 mg, 0.542 mmol) in THF (4 mL) added and the mixture stirred at −30° C. for 20 min and then at rt overnight. The reaction was evaporated to dryness in vacuo and the residue diluted with water (10 mL) containing acetic acid (98 mg, 1.63 mmol) and extracted with EtOAc (2×15 mL). The combined extracts were evaporated to dryness in vacuo and the residue purified by preparative HPLC (SunFire 100*19 mm 5 μm; H₂O-MeOH; 58%) to afford N-(6-(difluoromethyl)pyridin-2-yl)-8-methoxy-2-(1-methoxycyclopropyl)imidazo[1,2-a]pyridine-6-carboxamide (29.8 mg, 14% yield). LCMS m/z=389 (M+H)⁺; ¹H NMR (400 MHz, MeOH-d₄) δ: 1.21 (s, 4H), 3.40 (s, 3H), 4.09 (s, 3H), 6.66 (t, 1H), 7.21 (s, 1H), 7.44 (d, 1H), 7.93 (s, 1H), 7.99 (t, 1H), 8.39 (d, 1H), 8.78 (s, 1H).

### Example 92: N-(6-(difluoromethyl)pyridin-2-yl)-7-isopropoxy-2-(tetrahydro-2H-pyran-4-yl)imidazo[1,2-a]pyridine-6-carboxamide

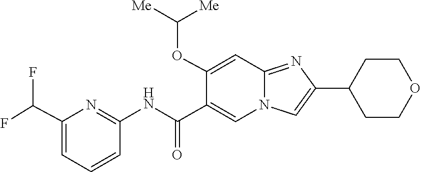 [SMILES: CC(C)Oc1cc2nc(C3CCOCC3)cn2cc1C(=O)Nc1cccc(C(F)F)n1]

n-BuLi (0.6 mL, 2.5M in hexane) was added to 6-(difluoromethyl)pyridine-2-amine (100 mg, 0.7 mmol) in THF (3 mL) at −78° C. and the mixture was stirred for 30 min before methyl 7-isopropoxy-2-(tetrahydro-2H-pyran-4-yl)imidazo[1,2-a]pyridine-6-carboxylate (Preparation 59, 53 mg, 0.2 mmol) in THF (4 mL) added and the mixture stirred at rt overnight. The reaction was evaporated to dryness in vacuo and the purified by preparative HPLC (SunFire 100*19 mm 5 μm; H₂O-MeOH; 58%) to afford N-(6-(difluoromethyl)pyridin-2-yl)-7-isopropoxy-2-(tetrahydro-2H-pyran-4-yl)imidazo[1,2-a]pyridine-6-carboxamide (10.4 mg, 12% yield). LCMS m/z=431 (M+H)⁺; ¹H NMR (400 MHz, CDCl₃) δ: 1.39-1.63 (m, 6H), 1.75-1.89 (m, 2H), 2.02 (d, 2H), 2.87-3.00 (m, 1H), 3.55 (t, 2H), 4.06 (d, 2H), 4.62-4.95 (m, 1H), 6.47 (t, 1H), 6.92 (s, 1H), 7.28 (s, 1H), 7.37 (d, 1H), 7.86 (t, 1H), 8.43 (d, 1H), 9.00 (s, 1H), 10.72 (s, 1H).

### Example 93: 7-isopropoxy-N-(6-methoxypyridin-2-yl)-2-(tetrahydro-2H-pyran-4-yl)imidazo[1,2-a]pyridine-6-carboxamide

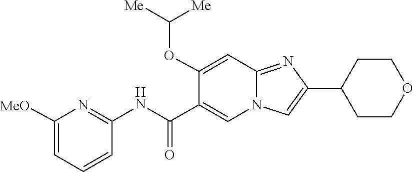 [SMILES: COc1cccc(NC(=O)c2cn3cc(C4CCOCC4)nc3cc2OC(C)C)n1]

n-BuLi (0.4 mL, 2.5M in hexane) was added to 6-methoxypyridine-2-amine (156 mg, 0.491 mmol) in THF (5 mL) at −78° C. and the mixture was stirred for 30 min before methyl 7-isopropoxy-2-(tetrahydro-2H-pyran-4-yl)imidazo[1,2-a]pyridine-6-carboxylate (Preparation 59, 100 mg, 0.3 mmol) in THF (4 mL) added and the mixture stirred at rt overnight. The reaction was evaporated to dryness in vacuo and the purified by preparative HPLC (XBridge C18 100×19 mm, 5 μm, MeOH/H₂O+0.1% NH₄OH modifier; gradient (% organic) 0-100) to afford 7-isopropoxy-N-(6-methoxypyridin-2-yl)-2-(tetrahydro-2H-pyran-4-yl)imidazo[1,2-a]pyridine-6-carboxamide (24.4 mg, 12% yield). LCMS m/z=411 [M+H]⁺; ¹H NMR (400 MHz, CDCl₃) δ: 1.49 (d, 6H), 1.59-1.73 (m, 2H), 1.90 (d, 2H), 2.82-2.97 (m, 1H), 3.46 (td, 2H), 3.83 (s, 3H), 3.87-3.96 (m, 2H), 4.89-5.15 (m, 1H), 6.60 (d, 1H), 7.17 (s, 1H), 7.60-7.85 (m, 3H), 9.17 (s, 1H), 10.63 (s, 1H),

### Example 94: 2-(8-oxabicyclo[3.2.1]octan-3-yl)-7-isopropoxy-N-(6-methoxypyridin-2-yl)imidazo[1,2-a]pyridine-6-carboxamide

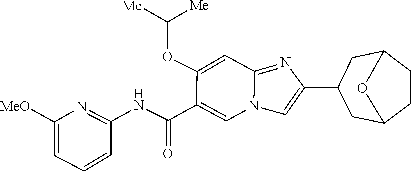 [SMILES: COc1cccc(NC(=O)c2cn3cc(C4CC5CCC(C4)O5)nc3cc2OC(C)C)n1]

n-BuLi (0.28 mL, 2.5M in hexane) was added to 6-methoxypyridine-2-amine (103 mg, 0.83 mmol) in THF (40 mL) at −78° C. under Ar. The mixture was stirred at −78° C. for 30 min and a solution of methyl 2-(8-oxabicyclo[3.2.1]octan-3-yl)-7-isopropoxyimidazo[1,2-a]pyridine-6-carboxylate (Preparation 117, 95 mg, 0.276 mmol) in THF (10 mL) added and the mixture stirred at rt overnight. The reaction mixture was diluted with NH₄C1 and the organic phase evaporated to dryness in vacuo and the residue purified by preparative HPLC (XBridge C18 100×19 mm, 5 μm, gradient 0-100% MeOH with H₂O and 0.1% NH₄OH modifier) to afford 2-(8-oxabicyclo[3.2.1]octan-3-yl)-7-isopropoxy-N-(6-methoxypyridin-2-yl)imidazo[1,2-a]pyridine-6-carboxamide (86 mg, 71% yield): LCMS m/z=437 [M+H]⁺; ¹H NMR (400 MHz, DMSO-d₆) δ: 1.57 (d, 6H), 1.64-1.96 (m, 6H), 1.97-2.04 (m, 2H), 3.15-3.25 (m, 1H), 3.85 (d, 3H), 4.48 (br s, 2H), 4.74-4.86 (m, 1H), 6.49 (d, 1H), 6.88 (s, 1H), 7.23 (s, 1H), 7.59 (dd, 1H), 7.85 (d, 1H), 8.98 (s, 1H), 10.50 (s, 1H).

### Example 95: 2-(Difluoromethyl)-N-(6-(difluoromethyl)pyridin-2-yl)-7-ethoxyimidazo[1,2-a]pyridine-6-carboxamide

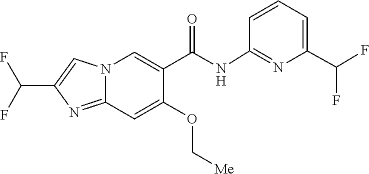 [SMILES: CCOc1cc2nc(C(F)F)cn2cc1C(=O)Nc1cccc(C(F)F)n1]

Part A. A mixture of methyl 2-(difluoromethyl)-7-ethoxyimidazo[1,2-a]pyridine-6-carboxylate (Preparation 55, 100 mg, 370 μmol) and LiOH (17.7 mg, 0.74 mmol) in THF (2 mL) and H₂O (2 mL) was stirred at 60° C. overnight. The reaction mixture was evaporated to dryness in vacuo and the residue dissolved in H₂O (5 mL) and washed with DCM (5 mL). The aqueous layer was acidified to pH 4-5 and evaporated to dryness afford 2-(difluoromethyl)-7-ethoxyimidazo[1,2-a]pyridine-6-carboxylic acid as a mixture with LiCl which was used in Part B without further purification. LCMS m/z=257 [M+H]⁺

Part B. A mixture of 2-(difluoromethyl)-7-ethoxyimidazo[1,2-a]pyridine-6-carboxylic acid (Part A; 120 mg, 0.468 mmol), 6-(difluoromethyl)pyridine-2-amine (67.5 mg, 0.468 mmol), 2-chloro-1-methyl-pyridin-1-ium iodide (239 mg, 0.937 mmol), TEA (142 mg, 1.41 mmol) and MeCN (10 mL) was stirred at 70° C. overnight. The reaction mixture was evaporated to dryness in vacuo, diluted with H₂O and extracted with CHCl₃ (3×10 mL). The combined organics were dried (Na₂SO₄) and evaporated to dryness in vacuo and the residue purified by preparative HPLC (XBridge C18 100*19 mm 5 μm; NH₄OH-MeOH/NH₃; % organic: 50-100) to afford 2-(difluoromethyl)-N-(6-(difluoromethyl)pyridin-2-yl)-7-ethoxyimidazo[1,2-a]pyridine-6-carboxamide (8.4 mg 4.7%): LCMS=m/z=383 [M+H]⁺; ¹H NMR (400 MHz, MeOH-d₄) δ: 1.63 (t, 3H), 4.27-4.47 (m, 2H), 6.44-7.00 (m, 2H), 7.06 (s, 1H), 7.42 (d, 1H), 7.98 (t, 1H), 8.05 (s, 1H), 8.40 (d, 1H), 9.12 (s, 1H).

### Example 96: N-(6-(difluoromethyl)pyridin-2-yl)-7-isopropoxy-2-(1-methyl-2-oxabicyclo[2.1.1]hexan-4-yl)imidazo[1,2-a]pyridine-6-carboxamide

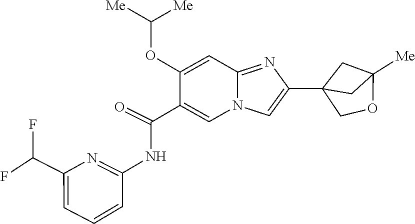 [SMILES: CC(C)Oc1cc2nc(C34COC(C)(C3)C4)cn2cc1C(=O)Nc1cccc(C(F)F)n1]

A mixture of 7-isopropoxy-2-(1-methyl-2-oxabicyclo[2.1.1]hexan-4-yl)imidazo[1,2-a]pyridine-6-carboxylic acid (Preparation 78, 85 mg, 0.269 mmol), 6-(difluoromethyl)pyridin-2-amine (38.7 mg, 0.269 mmol), 2-chloro-1-methyl-pyridin-1-ium iodide (137.3 mg, 0.538 mmol) and TEA (81.57 mg, 0.806 mmol) in MeCN (2 mL) was heated at 70° C. overnight. The reaction mixture was evaporated to dryness in vacuo and the residue dissolved in EtOAc (2 mL) and washed (NaHCO₃, 5 mL). The combined organics were evaporated to dryness in vacuo and purified by HPLC (XBridge C18 100×19 mm, 5 μm, gradient 0-100% MeOH with H₂O and 0.1% NH₄OH modifier) to afford N-(6-(difluoromethyl)pyridin-2-yl)-7-isopropoxy-2-(1-methyl-2-oxabicyclo[2.1.1]hexan-4-yl)imidazo[1,2-a]pyridine-6-carboxamide (45.1 mg, 38% yield). LCMS m/z=443 [M+H]⁺; ¹H NMR (400 MHz, DMSO-d₆) δ: 1.16-1.58 (m, 9H), 1.75 (dd, 2H), 1.99 (dd, 2H), 3.88 (s, 2H), 4.82-5.04 (m, 1H), 6.89 (t, 1H), 7.16 (s, 1H), 7.47 (d, 1H), 7.79 (s, 1H), 8.07 (t, 1H), 8.36 (d, 1H), 9.10 (s, 1H), 10.86 (s, 1H).

### Example 97: 7-Isopropoxy-N-(6-methoxypyridin-2-yl)-2-(1-methyl-2-oxabicyclo[2.1.1]hexan-4-yl)imidazo[1,2-a]pyridine-6-carboxamide

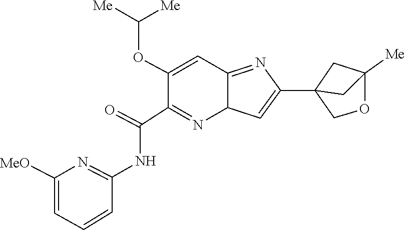 [SMILES: COc1cccc(NC(=O)C2=NC3C=C(C45COC(C)(C4)C5)N=C3C=C2OC(C)C)n1]

A mixture of 7-isopropoxy-2-(1-methyl-2-oxabicyclo[2.1.1]hexan-4-yl)imidazo[1,2-a]pyridine-6-carboxylic acid (Preparation 78, 85 mg, 0.269 mmol), 6-methoxypyridin-2-amine (33.4 mg, 0.269 mmol), 2-chloro-1-methyl-pyridin-1-ium iodide (137.3 mg, 0.538 mmol) and TEA (81.6 mg, 0.806 mmol) in MeCN (2 mL) was heated at 70° C. overnight. The reaction mixture was evaporated to dryness in vacuo and the residue dissolved in EtOAc (5 mL) and washed (NaHCO₃, 3 mL). The combined organics were evaporated to dryness in vacuo and purified by HPLC (XBridge C18 100×19 mm, 5 μm, gradient 0-100% MeOH with H₂O and 0.1% NH₄OH modifier) to afford 7-isopropoxy-N-(6-methoxypyridin-2-yl)-2-(1-methyl-2-oxabicyclo[2.1.1]hexan-4-yl)imidazo[1,2-a]pyridine-6-carboxamide (37 mg, 31% yield). LCMS m/z=423 [M+H]⁺; ¹H NMR (400 MHz, DMSO-d₆) δ: 1.42 (s, 3H), 1.49 (d, 6H), 1.73-1.78 (m, 2H), 1.96-2.02 (m, 2H), 3.83 (s, 3H), 3.88 (s, 2H), 4.93-5.06 (m, 1H), 6.60 (d, 1H), 7.19 (s, 1H), 7.58-7.88 (m, 3H), 9.17 (s, 1H), 10.61 (s, 1H).

### Example 98: 2-(1,1-Difluoroethyl)-N-(6-(difluoromethyl)pyridin-2-yl)-7-isopropoxyimidazo[1,2-a]pyridine-6-carboxamide

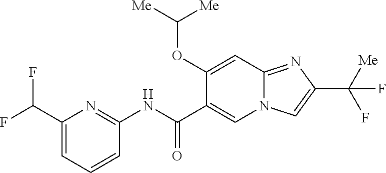 [SMILES: CC(C)Oc1cc2nc(C(C)(F)F)cn2cc1C(=O)Nc1cccc(C(F)F)n1]

A mixture of 2-(1,1-difluoroethyl)-7-isopropoxyimidazo[1,2-a]pyridine-6-carboxylic acid (Preparation 77, 350 mg, 1.23 mmol), 6-(difluoromethyl)pyridin-2-amine (177 mg, 1.23 mmol), 2-chloro-1-methyl-pyridin-1-ium iodide (628 mg, 2.46 mmol) and TEA (373 mg, 3.69 mmol) in MeCN (10 mL) was heated at 70° C. overnight. The reaction mixture was diluted with H₂O (20 mL) and extracted with EtOAc (4×25 mL). The combined extracts were washed (H₂O, 50 mL and brine, 50 mL), dried (Na₂SO₄), evaporated to dryness in vacuo and the residue purified by HPLC (XBridge C18 100×19 mm, 5 μm, gradient 0-100% MeOH with H₂O and 0.1% NH₄OH modifier) to afford 2-(1,1-difluoroethyl)-N-(6-(difluoromethyl)pyridin-2-yl)-7-isopropoxyimidazo[1,2-a]pyridine-6-carboxamide as a white solid (77 mg, 15%). LCMS m/z=411 [M+H]⁺; ¹H NMR (400 MHz, DMSO-d₆) δ: 1.42 (d, 6H), 2.01 (t, 3H), 4.92-4.95 (m, 1H), 6.88 (t, 1H), 7.21 (s, 1H), 7.46 (d, 1H), 8.04-8.09 (m, 1H), 8.15 (s, 1H), 8.35 (br. s, 1H), 9.13 (s, 1H), 10.88 (s, 1H).

### Examples 99-101

The title compounds were prepared in an analogous manner to that described for Example 98 using the appropriate carboxylic acid and amine as shown in the following table:

| Example | Name/Structure/Acid/Amine | QC Data/Yield |
|---|---|---|
| 99 | 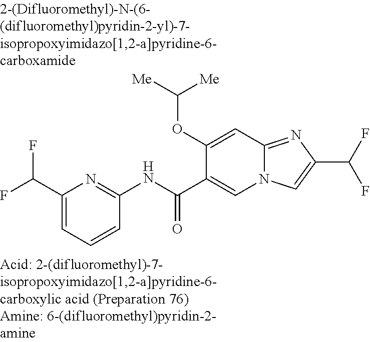 [SMILES: CC(C)Oc1cc2nc(C(F)F)cn2cc1C(=O)Nc1cccc(C(F)F)n1] | XBridge C18 100 × 19 mm, 5 μm, gradient 0-100% MeOH with H₂O and 0.1% NH₄OH modifier. White solid (29 mg, 6%). LCMS m/z = 397 [M + H]⁺ ¹H NMR (400 MHz, DMSO-d₆) δ: 1.43 (d, 6H), 4.87-4.98 (m, 1H), 6.69-7.32 (m, 3H), 7.48 (d, 1H), 8.08 (t, 1H), 8.21 (s, 1H), 8.36 (d, 1H), 9.17 (s, 1H), 10.91 (s, 1H). |
| 100 | 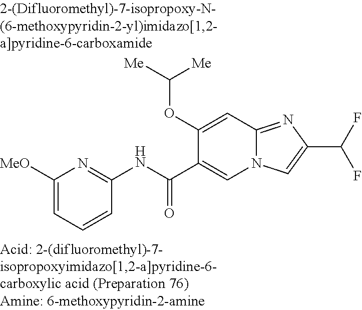 [SMILES: COc1cccc(NC(=O)c2cn3cc(C(F)F)nc3cc2OC(C)C)n1] | XBridge C18 100 × 19 mm, 5 μm, gradient 0-100% MeOH with H₂O and 0.1% NH₄OH modifier. White solid (44 mg, 11%). LCMS m/z = 377 [M + H]⁺ ¹H NMR (400 MHz, DMSO-d₆) δ: 1.49 (d, 6H), 3.84 (s, 3H), 4.86-5.15 (m, 1H), 6.61 (d, 1H), 6.92-7.26 (m, 2H), 7.71-7.90 (m, 2H), 8.25 (s, 1H), 9.28 (s, 1H), 10.61 (s, 1H). |
| 101 | 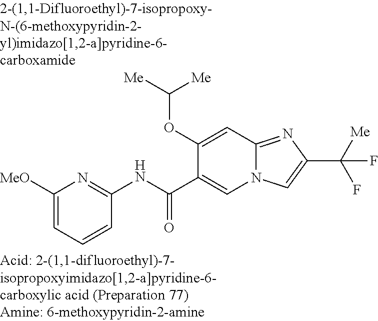 [SMILES: COc1cccc(NC(=O)c2cn3cc(C(C)(F)F)nc3cc2OC(C)C)n1] | XBridge C18 100 × 19 mm, 5 μm, gradient 0-100% MeOH with H₂O and 0.1% NH₄OH modifier. White solid (61 mg, 13%). LCMS m/z = 391 [M + H]⁺ ¹H NMR (400 MHz, DMSO-d₆) δ: 1.49 (d, 6H), 2.02 (t, 3H), 3.84 (s, 3H), 4.87-5.13 (m, 1H), 6.60 (d, 1H), 7.27 (s, 1H), 7.65-7.90 (m, 2H), 8.20 (s, 1H), 9.25 (s, 1H), 10.60 (s, 1H). |

### Example 102: N-(6-(difluoromethyl)pyridin-2-yl)-7-isopropoxy-2-(3-methoxypropyl)imidazo[1,2-a]pyridine-6-carboxamide

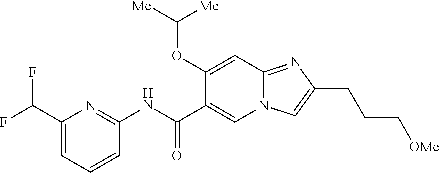 [SMILES: COCCCc1cn2cc(C(=O)Nc3cccc(C(F)F)n3)c(OC(C)C)cc2n1]

Part A. A solution of NaOH (12.7 mg, 0.309 mmol) in H₂O (2 mL) was added to methyl 7-isopropoxy-2-(3-methoxypropyl)imidazo[1,2-a]pyridine-6-carboxylate (Preparation 61, 73 mg, 0.237 mmol) MeOH (3 mL) and the mixture stirred at rt for 24 h. HCl (10 M, 0.31 mL, 0.31 mmol) was added and the mixture evaporated to dryness and used in Part B without any purification.

Part B. 6-(difluoromethyl)pyridin-2-amine (17.3 mg, 0.12 mmol), 2-chloro-1-methyl-pyridin-1-ium iodide (61.2 mg, 0.239 mmol) and TEA (36.4 mg, 0.359 mmol) were added to the residue from Part A in MeCN (2 mL) and the mixture stirred at 70° C. overnight. The cooled mixture was evaporated to dryness, dissolved in EtOAc (5 mL), washed (aq NaHCO₃, 3 mL), dried (Na₂SO₄) and evaporated to dryness in vacuo. The residue was purified by HPLC (XBridge C18 100×19 mm, 5 μm, gradient 0-100% MeOH with H₂O and 0.1% NH₄OH modifier) to afford N-(6-(difluoromethyl)pyridin-2-yl)-7-isopropoxy-2-(3-methoxypropyl)imidazo[1,2-a]pyridine-6-carboxamide (15 mg, 30% yield). LCMS m/z=419 [M+H]⁺; ¹H NMR (400 MHz, DMSO-d₆) δ: 1.44 (d, 6H), 1.73-2.00 (m, 2H), 2.66 (t, 2H), 3.24 (s, 3H), 3.37 (t, 2H), 4.91-5.01 (m, 1H), 6.89 (t, 1H), 7.10 (s, 1H), 7.47 (d, 1H), 7.69 (s, 1H), 8.07 (t, 1H), 8.37 (d, 1H), 9.10 (s, 1H), 10.86 (s, 1H).

### Example 103: 7-Isopropoxy-2-(3-methoxypropyl)-N-(6-methoxypyridin-2-yl)imidazo[1,2-a]pyridine-6-carboxamide

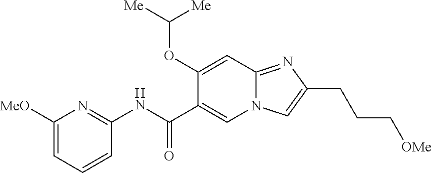 [SMILES: COCCCc1cn2cc(C(=O)Nc3cccc(OC)n3)c(OC(C)C)cc2n1]

7-isopropoxy-2-(3-methoxypropyl)-N-(6-methoxypyridin-2-yl)imidazo[1,2-a]pyridine-6-carboxamide was prepared in an analogous way to Example 107 using methyl 7-isopropoxy-2-(3-methoxypropyl)imidazo[1,2-a]pyridine-6-carboxylate (Preparation 61) and 6-methoxypyridin-2-amine. LCMS m/z=399 [M+H]⁺; ¹H NMR (400 MHz, DMSO-d₆) δ: 1.49 (d, 6H), 1.79-2.00 (m, 2H), 2.67 (t, 2H), 3.24 (s, 3H), 3.37 (t, 2H), 3.84 (s, 3H), 4.87-5.09 (m, 1H), 6.60 (d, 1H), 7.13 (s, 1H), 7.50-7.92 (m, 3H), 9.16 (s, 1H), 10.63 (s, 1H).

### Examples 104-116

The title compounds were prepared in parallel using the following protocol performed on a 100 mg scale (product). The appropriate amine (1.0 equiv.) and DIPEA (2.5 equiv. +1.0 equiv. per each acid equiv. for amine salts) was added to a mixture of 7-methoxy-2-(tetrahydro-2H-pyran-4-yl)imidazo[1,2-a]pyridine-6-carboxylic acid (Preparation 75) (1.0 eq) in anhydrous MeCN (0.5 mL). The resulting mixture was stirred for 5 min followed by the addition of Mukaiyama's reagent (1.0 eq) and the reaction stirred at 100° C. for 6 h. The resulting mixture was diluted with MeOH (1.0 mL) and stirred until a clear solution was observed, filtered and the filtrate purified by prep-HPLC (Waters SunFire C18 19×100 mm 5 μm; gradient mixture H₂O-MeOH or H₂O-MeCN as a mobile phase as stated in the following table).

| Example | Structure/Name/HPLC | Amine/Yield/Data |
|---|---|---|
| 104 | 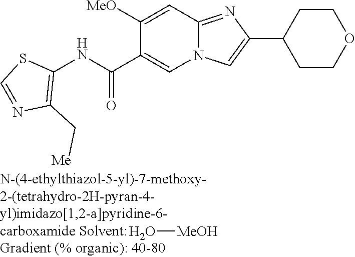 | 4-ethylthiazol-5-amine Yield: 12.7 mg LCMS m/z = 387 [M + H]⁺ ¹H NMR (400 MHz, CDCl₃) δ: 1.37 (t, 3H), 2.91-3.08 (m, 1H), 1.82 (qd, 2H), 2.03 (d, 2H), 2.81 (q, 2H), 3.50-3.60 (m, 2H), 4.02-4.10 (m, 2H), 4.13 (s, 3H), 7.13 (s, 1H), 7.30 (s, 1H), 8.43 (s, 1H), 9.03 (s, 1H), 10.22 (s, 1H). |
| 105 | 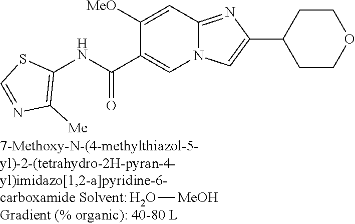 | 4-methylthiazol-5-amine Yield: 42.8 mg LCMS m/z = 373 [M + H]⁺ ¹H NMR (400 MHz, DMSO-d₆ + CCl₄) δ: 1.70-1.77 (m, 2H), 1.90-1.98 (m, 2H), 2.44 (s, 3H), 2.88-2.93 (m, 1H), 3.47 (t, 2H), 3.91- 3.98 (m, 2H), 4.09 (s, 3H), 7.04 (s, 1H), 7.58 (s, 1H), 8.43 (s, 1H), 9.08 (s, 1H), 10.34 (s, 1H), |
| 106 | 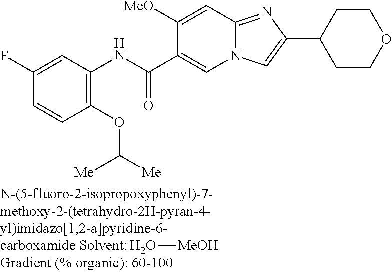 | 5-fluoro-2-isopropoxyaniline Yield: 9.0 mg LCMS m/z = 428 [M + H]⁺ ¹H NMR (500 MHz, DMSO-d₆) δ: 1.42 (d, 6H), 1.74 (qd, 2H), 1.91-1.97 (m, 2H), 2.86-2.94 (m, 1H), 3.43-3.52 (m, 2H), 3.91- 3.97 (m, 2H), 4.13 (s, 3H), 4.68 (hept, 1H), 6.74 (td, 1H), 6.98 (dd, 1H), 7.07 (s, 1H), 7.61 (s, 1H), 8.37 (dd, 1H), 9.18 (s, 1H), 10.29 (s, 1H). |
| 107 | 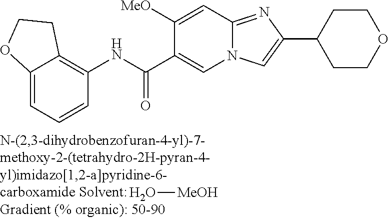 | 2,3-dihydrobenzofuran-4-amine Yield: 71.9 mg LCMS m/z = 394 [M + H]⁺ ¹H NMR (400 MHz, DMSO-d₆ + CCl₄) δ: 1.71-1.80 (m, 2H), 1.91-1.99 (m, 2H), 2.86-2.97 (m, 1H), 3.24 (t, 2H), 3.42-3.53 (m, 2H), 3.91-3.99 (m, 2H), 4.09 (s, 3H), 4.60 (t, 2H), 6.51 (d, 1H), 7.01-7.10 (m, 2H), 7.54 (d, 1H), 7.60 (s, 1H), 9.09 (s, 1H), 9.69 (s, 1H). |
| 108 | 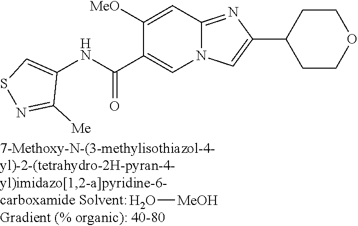 | 3-methylisothiazol-4-amine Yield: 15.9 mg LCMS m/z = 373 [M + H]⁺ ¹H NMR (400 MHz, DMSO-d₆) δ: 1.63-1.71 (m, 2H), 1.87-1.95 (m, 2H), 2.47 (s, 3H), 2.88-2.93 (m, 1H), 3.41-3.52 (m, 2H), 3.89-3.96 (m, 2H), 4.01 (s, 3H), 7.10 (s, 1H), 7.69 (s, 1H), 9.04 (s, 1H), 9.10 (s, 1H), 10.10 (s, 1H). |
| 109 | 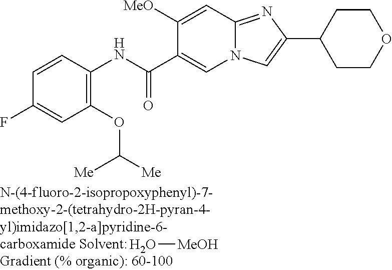 | 4-fluoro-2-isopropoxyaniline Yield: 21.8 mg LCMS m/z = 428 [M + H]⁺ ¹H NMR (400 MHz, DMSO-d₆ + CCl₄) δ: 1.45 (d, 6H), 1.68-1.81 (m, 2H), 1.91-1.99 (m, 2H), 2.89-2.94 (m, 1H), 3.48 (t, 2H), 3.91-3.98 (m, 2H), 4.14 (s, 3H), 4.75 (hept, 1H), 6.60-6.69 (m, 1H), 6.82- 6.90 (m, 1H), 7.09 (s, 1H), 7.62 (s, 1H), 8.48-8.56 (m, 1H), 9.18 (s, 1H), 10.10 (s, 1H). |
| 110 | 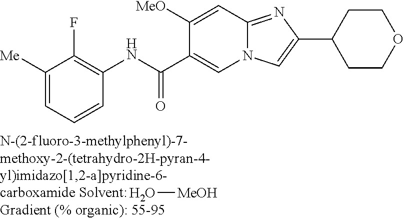 | 2-fluoro-3-methylaniline Yield: 35.6 mg LCMS m/z = 384 [M + H]⁺ ¹H NMR (400 MHz, DMSO-d₆ + CCl₄) δ: 1.67-1.81 (m, 2H), 1.90-1.98 (m, 2H), 2.32 (s, 3H), 2.84-2.92 (m, 1H), 3.42-3.52 (m, 2H), 3.91-3.98 (m, 2H), 4.12 (s, 3H), 6.93 (t, 1H), 6.99-7.07 (m, 2H), 7.59 (s, 1H), 8.25 (t, 1H), 9.15 (s, 1H), 10.10-10.15 (m, 1H). |
| 111 | 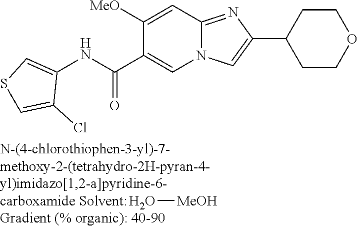 | 4-chlorothiophen-3-amine Yield: 2.4 mg LCMS m/z = 392 [M + H]⁺ |
| 112 | 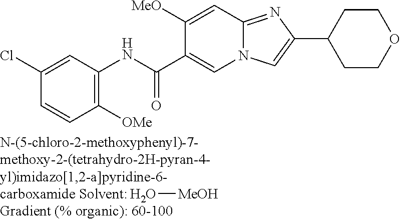 | 5-chloro-2-methoxyaniline Yield: 14.2 mg LCMS m/z = 416 [M + H]⁺ ¹H NMR (400 MHz, DMSO-d₆ + CCl₄) δ: 1.67-1.80 (m, 2H), 1.90-1.98 (m, 2H), 2.91 (s, 1H), 3.48 (t, 2H), 3.91-3.97 (m, 2H), 3.99 (s, 3H), 4.13 (s, 3H), 6.96-7.05 (m, 2H), 7.06 (s, 1H), 7.63 (s, 1H), 8.51- 8.56 (m, 1H), 9.17 (s, 1H), 10.52 (s, 1H). |
| 113 | 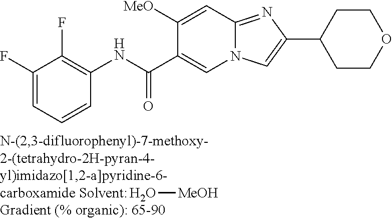 | 2,3-difluoroaniline Yield: 13.9 mg LCMS m/z = 388 [M + H]⁺ ¹H NMR (400 MHz, DMSO-d₆ + CCl₄) δ: 1.67-1.80 (m, 2H), 1.94 (d, 2H), 2.87-2.92 (m, 1H), 3.42-3.52 (m, 2H), 3.91-3.98 (m, 2H), 4.11 (s, 3H), 6.96-7.04 (m, 1H), 7.06 (s, 1H), 7.12-7.17 (m, 1H), 7.59 (s, 1H), 8.18 (t, 1H), 9.14 (s, 1H), 10.20 (s, 1H), |
| 114 | 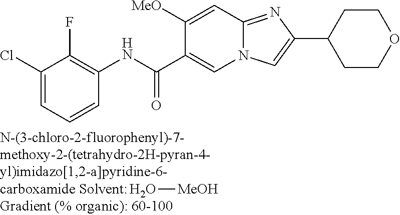 | 3-chloro-2-fluoroaniline Yield: 65.1 mg LCMS m/z = 404 [M + H]⁺ ¹H NMR (400 MHz, DMSO-d₆) δ: 1.61-1.73 (m, 2H), 1.87-1.95 (m, 2H), 2.88-2.93 (m, 1H), 3.41-3.51 (m, 2H), 3.92 (d, 2H), 4.01 (s, 3H), 7.10 (s, 1H), 7.27 (t, 1H), 7.35-7.44 (m, 1H), 7.70 (s, 1H), 8.03-8.11 (m, 1H), 9.06 (s, 1H), 10.21 (s, 1H). |
| 115 | 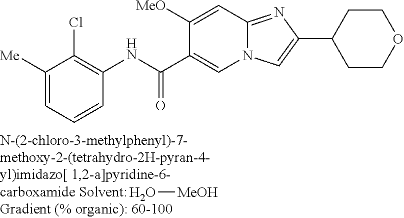 | 2-chloro-3-methylaniline Yield: 10.8 mg LCMS m/z = 400 [M + H]⁺ ¹H NMR (500 MHz, DMSO-d₆ + CCl₄) δ: 1.74 (qd, 2H), 1.90-1.97 (m, 2H), 2.43 (s, 3H), 2.86-2.94 (m, 1H), 3.43-3.51 (m, 2H), 3.92-3.98 (m, 2H), 4.15 (s, 3H), 7.04 (d, 1H), 7.07 (s, 1H), 7.21 (t, 1H), 7.61 (s, 1H), 8.43 (d, 1H), 9.19 (s, 1H), 10.47 (s, 1H). |
| 116 | 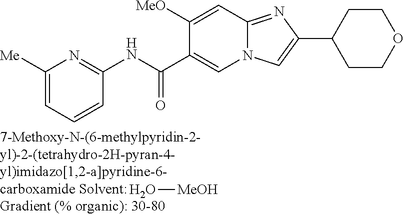 | 6-methylaniline Yield: 19 mg; 19% LCMS m/z = 367 [M + H]⁺ ¹H NMR (400 MHz, DMSO-d₆ + CCl₄) δ: 1.66-1.81 (m, 2H), 1.90-1.97 (m, 2H), 2.46 (s, 3H), 2.87-2.92 (m, 1H), 3.42-3.56 (m, 2H), 3.91-3.98 (m, 2H), 4.14 (s, 3H), 6.93 (d, 1H), 7.03 (s, 1H), 7.58 (s, 1H), 7.64 (t, 1H), 8.10 (d, 1H), 9.12 (s, 1H), 10.12 (s, 1H). |

### Examples 117-203

The title compounds were prepared in parallel using the following protocol performed on a 100 mg-scale (product). The appropriate amine (1.0 equiv.) was added to a mixture of 7-methoxy-2-(tetrahydro-2H-pyran-4-yl)imidazo[1,2-a]pyridine-6-carboxylic acid (Preparation 75, 1.0 equiv.), EDC (1 equiv.) and HOAt (1 equiv.) in DMSO (0.5 mL). The resulting mixture was stirred for 5 min followed by the addition of TEA (1.1 equiv. +1 equiv. per each acid equivalent for amine salts). The reaction mixture was stirred at rt for 24 h. After all starting materials were consumed, as was shown by LCMS, the resulting mixture was filtered and the filtrate purified by preparative HPLC (Waters SunFire C18 19×100 mm 5 μm; gradient mixture H₂O-MeOH or H₂O-MeCN as a mobile phase as stated in the following table) to afford the title compounds.

| Example | Structure/Name/HPLC | Amine/Yield/Data |
|---|---|---|
| 117 | 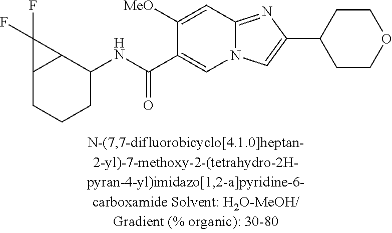 [SMILES: COc1cc2nc(C3CCOCC3)cn2cc1C(=O)NC1CCCC2C1C2(F)F] | 7,7-difluorobicyclo[4.1.0]heptan-2- amine Yield: 7.7 mg, 7% LCMS m/z = 406 [M + H]⁺ |
| 118 | 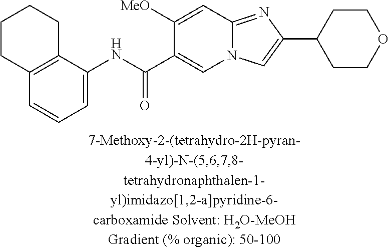 [SMILES: COc1cc2nc(C3CCOCC3)cn2cc1C(=O)Nc1cccc2c1CCCC2] | 5,6,7,8-tetrahydronaphthalen-1- amine Yield: 7.6 mg, 8% LCMS m/z = 406 [M + H]⁺ |
| 119 | 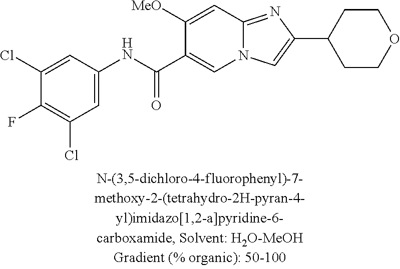 [SMILES: COc1cc2nc(C3CCOCC3)cn2cc1C(=O)Nc1cc(Cl)c(F)c(Cl)c1] | 3,5-dichloro-4-methylaniline Yield: 36 mg, 34% LCMS m/z = 438 [M + H]⁺ ¹H NMR (500 MHz, DMSO-d₆ + CCl₄) δ: 1.66-1.77 (m, 2H), 1.89-1.97 (m, 2H), 2.83-2.91 (m, 1H), 3.42-3.52 (m, 2H), 3.90- 3.98 (m, 2H), 4.02 (s, 3H), 6.95 (s, 1H), 7.54 (s, 1H), 7.96 (d, 2H), 8.89 (s, 1H), 10.10 (s, 1H). |
| 120 | 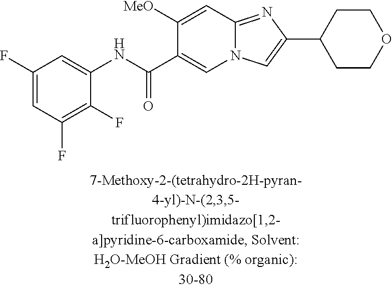 [SMILES: COc1cc2nc(C3CCOCC3)cn2cc1C(=O)Nc1cc(F)cc(F)c1F] | 2,3,5-trifluorolaniline Yield: 5.5 mg, 5% LCMS m/z = 406 [M + H]⁺ |
| 121 | 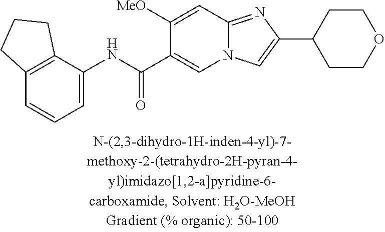 [SMILES: COc1cc2nc(C3CCOCC3)cn2cc1C(=O)Nc1cccc2c1CCC2] | 2,3-dihydro-1H-inden-4-amine Yield: 23 mg, 23% LCMS m/z = 392 [M + H]⁺ |
| 122 | 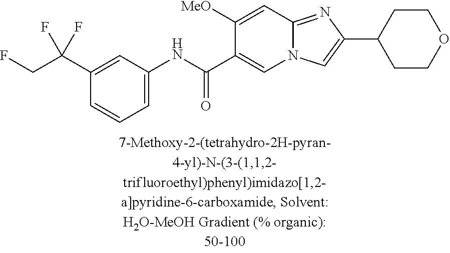 [SMILES: COc1cc2nc(C3CCOCC3)cn2cc1C(=O)Nc1cccc(C(F)(F)CF)c1] | 3-(1,1,2-trifluoroethyl)aniline Yield: 19 mg, 19% LCMS m/z = 434 [M + H]⁺ ¹H NMR (400 MHz, DMSO-d₆ + CCl₄) δ: 1.69-1.77 (m, 2H), 1.90-1.98 (m, 2H), 2.87-2.91 (m, 1H), 3.42-3.52 (m, 2H), 3.91- 3.98 (m, 2H), 4.04 (s, 3H), 4.72 (t, 1H), 4.84 (t, 1H), 6.96 (s, 1H), 7.27 (d, 1H), 7.46 (t, 1H), 7.53 (s, 1H), 7.91 (d, 1H), 7.99 (s, 1H), 8.91 (s, 1H), 10.11 (s, 1H). |
| 123 | 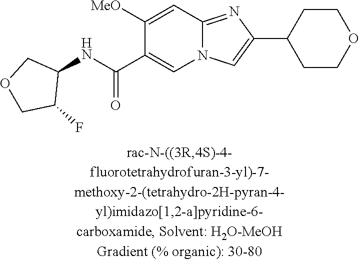 [SMILES: COc1cc2nc(C3CCOCC3)cn2cc1C(=O)N[C@@H]1COC[C@H]1F] | rac-(3R,4S)-4-fluorotetrahydrofuran- 3-amine Yield: 30%, 29% LCMS m/z = 364 [M + H]⁺ ¹H NMR (500 MHz, DMSO-d₆) δ: 1.66-1.78 (m, 2H), 1.89-1.95 (m, 2H), 2.82-2.91 (m, 1H), 3.42-3.52 (m, 2H), 3.52-3.59 (m, 1H), 3.90-3.97 (m, 2H), 3.97- 4.06 (m, 4H), 4.06-4.16 (m, 2H), 4.59-4.74 (m, 1H), 5.15-5.31 (m, 1H), 6.95 (s, 1H), 7.53 (s, 1H), 8.21 (d, 1H), 8.99 (s, 1H). |
| 124 | 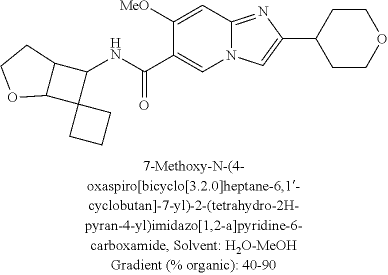 [SMILES: COc1cc2nc(C3CCOCC3)cn2cc1C(=O)NC1C2CCOC2C12CCC2] | 4-oxaspiro[bicyclo[3.2.0]heptane- 6,1′-cyclobutan]-7-amine Yield: 35 mg, 33% LCMS m/z = 412 [M + H]⁺ ¹H NMR (500 MHz, DMSO-d₆) δ: 1.66-1.78 (m, 5H), 1.78-1.88 (m, 3H), 1.90-1.95 (m, 2H), 2.05-2.21 (m, 2H), 2.78-2.84 (m, 1H), 2.84-2.91 (m, 1H), 3.19- 3.24 (m, 1H), 3.42-3.50 (m, 2H), 3.60-3.78 (m, 1H), 3.90-3.98 (m, 2H), 3.98-4.03 (m, 3H), 4.03-4.27 (m, 2H), 6.92-7.00 (m, 1H), 7.49- 7.56 (m, 1H), 7.83-8.04 (m, 1H), 8.89-9.00 (m, 1H). |
| 125 | 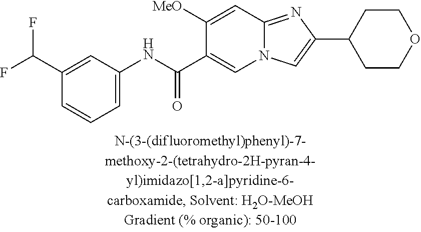 [SMILES: COc1cc2nc(C3CCOCC3)cn2cc1C(=O)Nc1cccc(C(F)F)c1] | 3-difluoromethylaniline Yield: 6.3 mg, 6.3% LCMS m/z = 402 [M + H]⁺ |
| 126 | 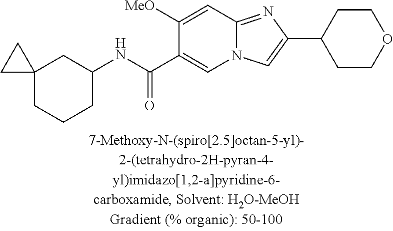 [SMILES: COc1cc2nc(C3CCOCC3)cn2cc1C(=O)NC1CCCC2(CC2)C1] | spiro[2.5]octan-5-amine Yield: 7.1 mg, 7% LCMS m/z = 384 [M + H]⁺ |
| 127 | 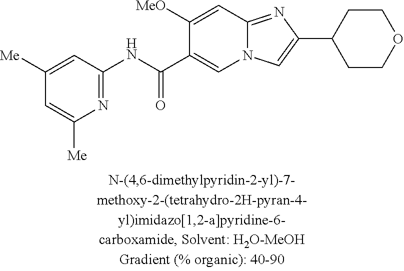 [SMILES: COc1cc2nc(C3CCOCC3)cn2cc1C(=O)Nc1cc(C)cc(C)n1] | 4,6-dimethylpyridin-2-amine Yield: 10.2 mg, 10% LCMS m/z = 381 [M + H]⁺ ¹H NMR (500 MHz, DMSO-d₆) δ: 1.67-1.80 (m, 2H), 1.90-1.97 (m, 2H), 2.36 (s, 3H), 2.40 (s, 3H), 2.89 (tt, 1H), 3.43-3.51 (m, 2H), 3.91-3.98 (m, 2H), 4.14 (s, 3H), 6.76 (s, 1H), 7.02 (s, 1H), 7.58 (s, 1H), 7.93 (s, 1H), 9.08 (s, 1H), 10.05 (s, 1H). |
| 128 | 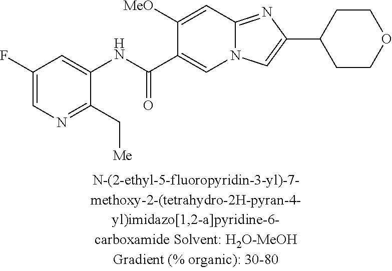 [SMILES: CCc1ncc(F)cc1NC(=O)c1cn2cc(C3CCOCC3)nc2cc1OC] | 2-ethyl-5-fluoropyridin-3-amine Yield: 10.6 mg, 11.7% LCMS m/z = 399 [M + H]⁺ |
| 129 | 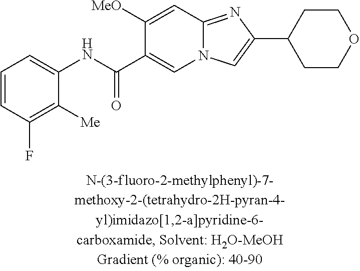 [SMILES: COc1cc2nc(C3CCOCC3)cn2cc1C(=O)Nc1cccc(F)c1C] | 3-fluoro-2-methylaniline Yield: 7.1 mg, 6.7% LCMS m/z = 384 [M + H]⁺ |
| 130 | 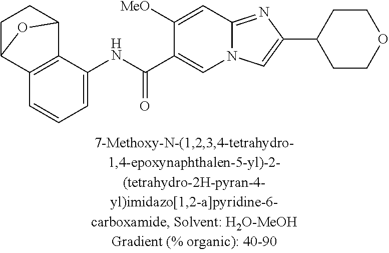 [SMILES: COc1cc2nc(C3CCOCC3)cn2cc1C(=O)Nc1cccc2c1C1CCC2O1] | 1,2,3,4-tetrahydro-1,4- epoxynaphthalen-5-amine Yield: 8.1 mg, 8.5% LCMS m/z = 420 [M + H]⁺ |
| 131 | 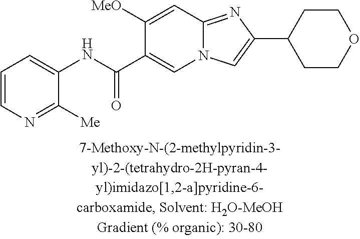 [SMILES: COc1cc2nc(C3CCOCC3)cn2cc1C(=O)Nc1cccnc1C] | 2-methylpyridin-3-amine Yield: 36.4 mg, 33% LCMS m/z = 367 [M + H]⁺ ¹H NMR (400 MHz, DMSO-d₆ + CCl₄) δ: 1.67-1.81 (m, 2H), 1.90-1.98 (m, 2H), 2.56 (s, 3H), 2.81-2.94 (m, 1H), 3.42-3.53 (m, 2H), 3.91-3.98 (m, 2H), 4.11 (s, 3H), 7.04 (s, 1H), 7.19 (dd, 1H), 7.58 (s, 1H), 8.19 (dd, 1H), 8.39 (d, 1H), 9.11 (s, 1H), 9.81 (s, 1H). |
| 132 | 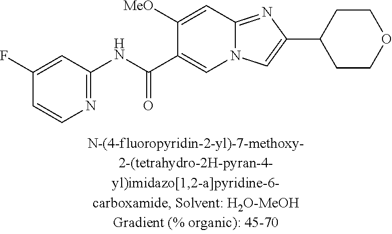 [SMILES: COc1cc2nc(C3CCOCC3)cn2cc1C(=O)Nc1cc(F)ccn1] | 4-fluoropyridin-2-amine Yield: 8.3 mg, 8% LCMS m/z = 371 [M + H]⁺ |
| 133 | 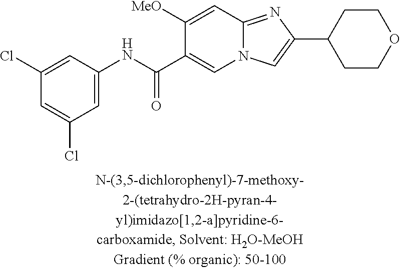 [SMILES: COc1cc2nc(C3CCOCC3)cn2cc1C(=O)Nc1cc(Cl)cc(Cl)c1] | 3,5-dichloroaniline Yield: 18 mg, 18% LCMS m/z = 420 [M + H]⁺ ¹H NMR (500 MHz, DMSO-d₆) δ: 1.73 (qd, 2H), 1.90- 1.96 (m, 2H), 2.88 (tt, 1H), 3.43-3.51 (m, 2H), 3.90-3.98 (m, 2H), 4.02 (s, 3H), 6.95 (s, 1H), 7.07-7.12 (m, 1H), 7.53 (s, 1H), 7.81-7.85 (m, 2H), 8.88 (s, 1H), 10.13 (s, 1H). |
| 134 | 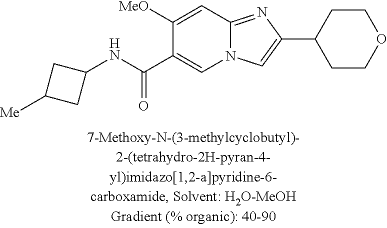 [SMILES: COc1cc2nc(C3CCOCC3)cn2cc1C(=O)NC1CC(C)C1] | 3-methylcyclobutylamine Yield: 12.3 mg, 13% LCMS m/z = 344 [M + H]⁺ ¹H NMR (500 MHz, DMSO-d₆) δ: 1.10-1.24 (m, 3H), 1.54-1.65 (m, 1H), 1.65-1.77 (m, 2H), 1.88-1.95 (m, 2H), 1.96-2.12 (m, 2H), 2.14-2.43 (m, 2H), 2.81- 2.91 (m, 1H), 3.41-3.51 (m, 2H), 3.89-3.96 (m, 2H), 4.00 (s, 3H), 4.19-4.59 (m, 1H), 6.90 (s, 1H), 7.48 (s, 1H), 7.89-8.03(m, 1H), 8.83-8.88 (m, 1H). |
| 135 | 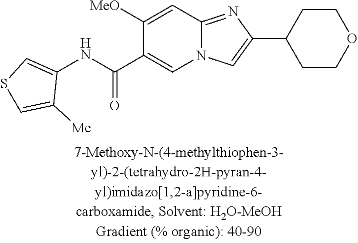 [SMILES: COc1cc2nc(C3CCOCC3)cn2cc1C(=O)Nc1cscc1C] | 4-methylthiophen-3-amine Yield: 6 mg, 6% LCMS m/z = 372 [M + H]⁺ |
| 136 | 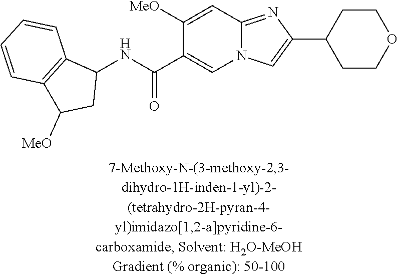 [SMILES: COc1cc2nc(C3CCOCC3)cn2cc1C(=O)NC1CC(OC)c2ccccc21] | 3-methoxy-2,3-dihydro-1H-inden-1- amine Yield: 14.5 mg, 15% LCMS m/z = 422 [M + H]⁺ ¹H NMR (400 MHz, CDCl₃) δ: 1.74-1.89 (m, 2H), 1.92- 2.02 (m, 3H), 2.82-2.93 (m, 1H), 2.93-3.00 (m, 1H), 3.47 (s, 3H), 3.49-3.59 (m, 2H), 3.85 (s, 3H), 4.01-4.09 (m, 2H), 4.70-4.77 (m, 1H), 5.57-5.65 (m, 1H), 6.83 (s, 1H), 7.24 (s, 1H), 7.28-7.35 (m, 2H), 7.40-7.47 (m, 2H), 8.24 (d, 1H), 8.95 (s, 1H). |
| 137 | 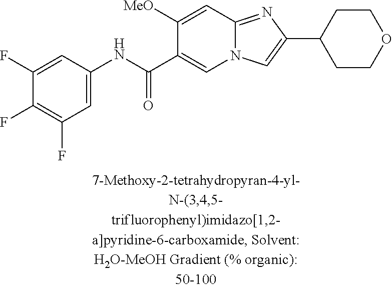 [SMILES: COc1cc2nc(C3CCOCC3)cn2cc1C(=O)Nc1cc(F)c(F)c(F)c1] | 3,4,5-trifluoroaniline Yield: 28.6 mg, 26% LCMS m/z = 406 [M + H]⁺ ¹H NMR (400 MHz, DMSO-d₆ + CCl₄) δ: 1.73 (qd, 2H), 1.89-1.97 (m, 2H), 3.90-3.98 (m, 2H), 2.81-2.95 (m, 1H), 3.42-3.52 (m, 2H), 4.02 (s, 3H), 6.95 (s, 1H), 7.53 (s, 1H), 7.64- 7.73 (m, 2H), 8.90 (s, 1H), 10.14 (s, 1H). |
| 138 | 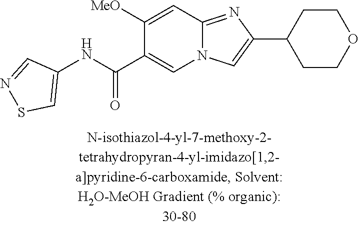 [SMILES: COc1cc2nc(C3CCOCC3)cn2cc1C(=O)Nc1cnsc1] | isothiazolamine Yield: 36 mg, 35% LCMS m/z = 359 [M + H]⁺ ¹H NMR (400 MHz, DMSO-d₆ + CCl₄) δ: 1.66-1.81 (m, 2H), 1.90-1.97 (m, 2H), 2.83-2.95 (m, 1H), 3.47 (td, 2H), 3.91-3.98 (m, 2H), 4.03 (s, 3H), 6.96 (s, 1H), 7.53 (s, 1H), 8.74 (s, 1H), 8.95 (s, 1H), 9.01 (s, 1H), 10.48 (s, 1H). |
| 139 | 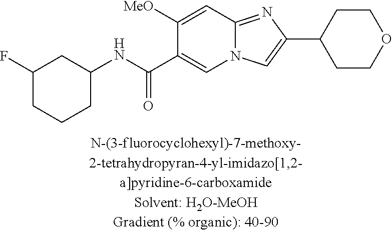 [SMILES: COc1cc2nc(C3CCOCC3)cn2cc1C(=O)NC1CCCC(F)C1] | 3-fluorocyclohexylamine Yield: 29 mg, 28% LCMS m/z = 376 [M + H]⁺ ¹H NMR (500 MHz, DMSO-d₆) δ: 1.35-1.53 (m, 2H), 1.53-1.68 (m, 2H), 1.69-1.79 (m, 3H), 1.80-1.98 (m, 4H), 2.14-2.17 (m, 1H), 2.82-2.91 (m, 1H), 3.41- 3.50 (m, 2H), 3.89-3.96 (m, 2H), 3.98 (s, 3H), 4.03-4.17 (m, 1H), 4.67-5.02 (m, 1H), 6.90 (s, 1H), 7.50 (s, 1H), 7.74-8.24 (m, 1H), 8.85-8.94 (m, 1H). |
| 140 | 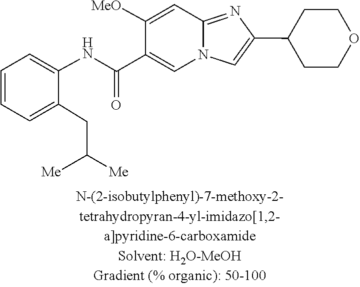 [SMILES: COc1cc2nc(C3CCOCC3)cn2cc1C(=O)Nc1ccccc1CC(C)C] | 2-isobutylaniline Yield: 15.5 mg, 15% LCMS m/z = 408 [M + H]⁺ ¹H NMR (400 MHz, CDCl₃) δ: 0.93 (d, 6H), 1.76-1.95 (m, 3H), 1.98-2.06 (m, 2H), 2.51 (d, 2H), 2.95-3.00 (m, 1H), 3.55 (t, 2H), 3.99-4.16 (m, 5H), 6.98 (s, 1H), 7.07-7.14 (m, 1H), 7.14-7.20 (m, 1H), 7.21-7.26 (m, 1H), 7.26-7.30 (m, 1H), 8.04 (d, 1H), 9.03 (s, 1H), 9.52 (s, 1H). |
| 141 | 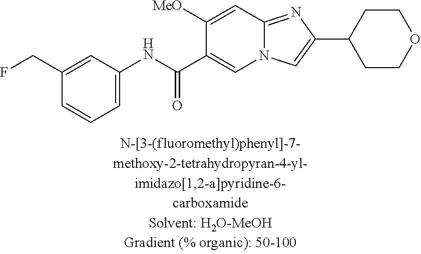 [SMILES: COc1cc2nc(C3CCOCC3)cn2cc1C(=O)Nc1cccc(CF)c1] | 3-fluoromethylaniline Yield: 18.4 mg, 19% LCMS m/z = 384 [M + H]⁺ ¹H NMR (500 MHz, DMSO-d₆) δ: 1.73 (qd, 2H), 1.90- 1.97 (m, 2H), 2.89 (tt, 1H), 3.43-3.51 (m, 2H), 3.90-3.98 (m, 2H), 4.04 (s, 3H), 5.38 (d, 2H), 6.96 (s, 1H), 7.10 (d, 1H), 7.35 (t, 1H), 7.53 (s, 1H), 7.71 (d, 1H), 7.77-7.81 (m, 1H), 8.92 (s, 1H), 9.98 (s, 1H). |
| 142 | 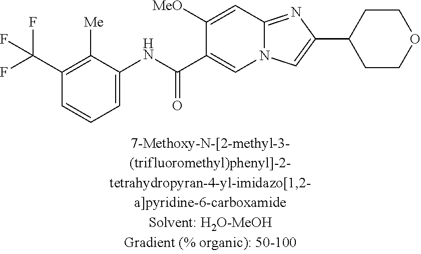 [SMILES: COc1cc2nc(C3CCOCC3)cn2cc1C(=O)Nc1cccc(C(F)(F)F)c1C] | 2-methyl-3-trifluoromethylaniline Yield: 19.5 mg, 19% LCMS m/z = 434 [M + H]⁺ ¹H NMR (400 MHz, DMSO-d₆ + CCl₄) δ: 1.67-1.81 (m, 2H), 1.90-1.98 (m, 2H), 2.44 (s, 3H), 2.87-2.91 (m, 1H), 3.42-3.52 (m, 2H), 3.91-3.98 (m, 2H), 4.09 (s, 3H), 7.03 (s, 1H), 7.38 (t, 1H), 7.48 (d, 1H), 7.57 (s, 1H), 8.16 (d, 1H), 9.09 (s, 1H), 9.84 (s, 1H). |
| 143 | 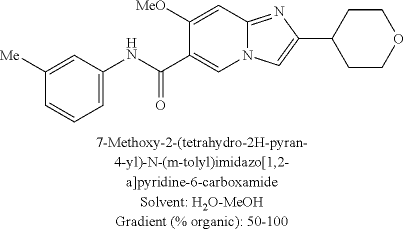 [SMILES: COc1cc2nc(C3CCOCC3)cn2cc1C(=O)Nc1cccc(C)c1] | 3-methylaniline Yield: 50 mg, 47% LCMS m/z = 366 [M + H]⁺ ¹H NMR (500 MHz, DMSO-d₆) δ: 1.67-1.79 (m, 2H), 1.90-1.97 (m, 2H), 2.37 (s, 3H), 2.84-2.93 (m, 1H), 3.43-3.51 (m, 2H), 3.90-3.98 (m, 2H), 4.05 (s, 3H), 6.87 (d, 1H), 6.96 (s, 1H), 7.17 (t, 1H), 7.47-7.55 (m, 3H), 8.92 (s, 1H), 9.80 (s, 1H). |
| 144 | 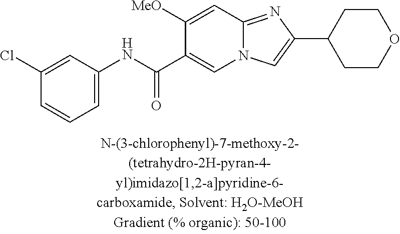 [SMILES: COc1cc2nc(C3CCOCC3)cn2cc1C(=O)Nc1cccc(Cl)c1] | 3-chloroaniline Yield: 49 mg, 47% LCMS m/z = 386 [M + H]⁺ ¹H NMR (400 MHz, DMSO-d₆ + CCl₄) δ: 1.67-1.80 (m, 2H), 1.94 (d, 2H), 2.86-2.91 (m, 1H), 3.47 (t, 2H), 3.94 (d, 2H), 4.03 (s, 3H), 6.95 (s, 1H), 7.06 (d, 1H), 7.29 (t, 1H), 7.53 (s, 1H), 7.62 (d, 1H), 7.91 (s, 1H), 8.91 (s, 1H), 10.03 (s, 1H). |
| 145 | 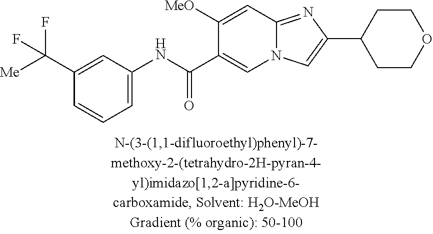 [SMILES: COc1cc2nc(C3CCOCC3)cn2cc1C(=O)Nc1cccc(C(C)(F)F)c1] | 3-(1,1-difluoroethyl)aniline Yield: 7.8 mg, 8% LCMS m/z = 416 [M + H]⁺ |
| 146 | 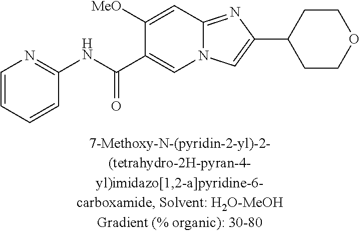 [SMILES: COc1cc2nc(C3CCOCC3)cn2cc1C(=O)Nc1ccccn1] | 2-aminopyridine Yield: 9.3 mg, 9.8% LCMS m/z = 353 [M + H]⁺ |
| 147 | 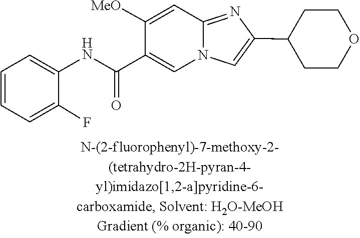 [SMILES: COc1cc2nc(C3CCOCC3)cn2cc1C(=O)Nc1ccccc1F] | 2-fluoroaniline Yield: 25.4 mg, 26.8% LCMS m/z = 370 [M + H]⁺ ¹H NMR (400 MHz, DMSO-d₆ + CCl₄) δ: 1.67-1.80 (m, 2H), 1.90-1.98 (m, 2H), 2.84-2.95 (m, 1H), 3.47 (t, 2H), 3.95 (d, 2H), 4.09-4.14 (m, 3H), 7.05 (s, 1H), 7.08-7.12 (m, 1H), 7.13-7.23 (m, 2H), 7.59 (s, 1H), 8.43 (t, 1H), 9.15 (s, 1H), 10.14 (s, 1H). |
| 148 | 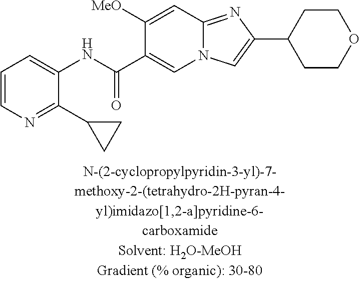 [SMILES: COc1cc2nc(C3CCOCC3)cn2cc1C(=O)Nc1cccnc1C1CC1] | 2-cyclopropylpyridin-3-amine Yield: 24 mg, 26% LCMS m/z = 393 [M + H]⁺ ¹H NMR (400 MHz, DMSO-d₆ + CCl₄) δ: 0.97-1.04 (m, 2H), 1.05-1.10 (m, 2H), 1.70-1.80 (m, 2H), 1.90-1.98 (m, 2H), 2.10- 2.19 (m, 1H), 2.85-2.92 (m, 1H), 3.42-3.53 (m, 2H), 3.91-3.98 (m, 2H), 4.08 (s, 3H), 7.04 (s, 1H), 7.12 (dd, 1H), 7.58 (s, 1H), 8.15-8.21 (m, 1H), 8.31 (d, 1H), 9.12 (s, 1H), 10.02 (s, 1H). |
| 149 | 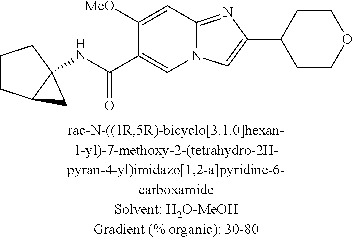 [SMILES: COc1cc2nc(C3CCOCC3)cn2cc1C(=O)N[C@@]12CCC[C@@H]1C2] | rac-(1R,5R)-bicyclo[3.1.0]hexan-1- amine hydrochloride Yield: 7.5 mg, 7% LCMS m/z = 356 [M + H]⁺ ¹H NMR (500 MHz, DMSO-d₆) δ: 0.73-0.79 (m, 2H), 1.17-1.29 (m, 1H), 1.40-1.47 (m, 1H), 1.61-1.77 (m, 4H), 1.88-1.95 (m, 2H), 1.97-2.02 (m, 2H), 1.99- 2.08 (m, 1H), 2.86 (tt, 1H), 3.41-3.50 (m, 2H), 3.89-3.96 (m, 2H), 3.98 (s, 3H), 6.89 (s, 1H), 7.49 (s, 1H), 8.09 (s, 1H), 8.86 (s, 1H). |
| 150 | 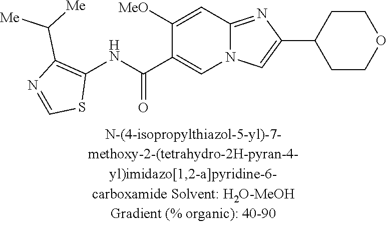 [SMILES: COc1cc2nc(C3CCOCC3)cn2cc1C(=O)Nc1scnc1C(C)C] | 4-isopropylthiazol-5-amine Yield: 9.3 mg, 9% LCMS m/z = 401 [M + H]⁺ ¹H NMR (500 MHz, DMSO-d₆) δ: 1.33 (d, 6H), 1.74 (qd, 2H), 1.91-1.97 (m, 2H), 2.85-2.94 (m, 1H), 3.15 (hept, 1H), 3.43-3.51 (m, 2H), 3.91-3.98 (m, 2H), 4.09 (s, 3H), 7.05 (s, 1H), 7.59 (s, 1H), 8.44 (s, 1H), 9.09 (s, 1H), 10.31 (s, 1H). |
| 151 | 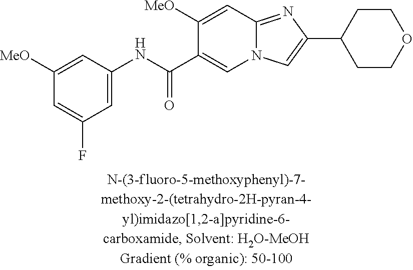 [SMILES: COc1cc(F)cc(NC(=O)c2cn3cc(C4CCOCC4)nc3cc2OC)c1] | 3-fluoro-5-methoxy aniline Yield: 9.5 mg, 10% LCMS m/z = 400 [M + H]⁺ |
| 152 | 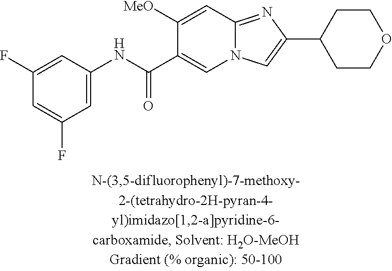 [SMILES: COc1cc2nc(C3CCOCC3)cn2cc1C(=O)Nc1cc(F)cc(F)c1] | 3,5-difluoroaniline Yield: 19.5 mg, 21% LCMS m/z = 388 [M + H]⁺ ¹H NMR (400 MHz, DMSO-d₆ + CCl4) δ: 1.73 (qd, 2H), 1.90-1.97 (m, 2H), 2.86-2.91 (m, 1H), 3.42-3.52 (m, 2H), 3.91-3.98 (m, 2H), 4.02 (s, 3H), 6.59-6.68 (m, 1H), 6.95 (s, 1H), 7.45-7.51 (m, 2H), 7.53 (s, 1H), 8.90 (s, 1H), 10.18 (s, 1H). |
| 153 | 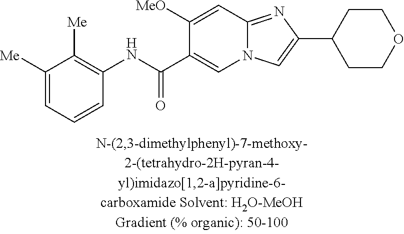 [SMILES: COc1cc2nc(C3CCOCC3)cn2cc1C(=O)Nc1cccc(C)c1C] | 2,3-dimethylaniline Yield: 9.7 mg, 9% LCMS m/z = 380 [M + H]⁺ ¹H NMR (500 MHz, DMSO-d₆) δ: 1.68-1.80 (m, 2H), 1.90-1.98 (m, 2H), 2.23 (s, 3H), 2.33 (s, 3H), 2.89 (tt, 1H), 3.43-3.51 (m, 2H), 3.91-3.98 (m, 2H), 4.09 (s, 3H), 6.96 (d, 1H), 7.02 (s, 1H), 7.06 (t, 1H), 7.56 (s, 1H), 7.70 (d, 1H), 9.08 (s, 1H), 9.64 (s, 1H). |
| 154 | 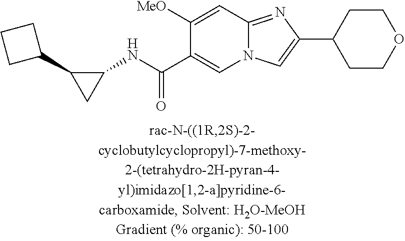 [SMILES: COc1cc2nc(C3CCOCC3)cn2cc1C(=O)N[C@@H]1C[C@H]1C1CCC1] | rac-(1R,2S)-2- cyclobutylcyclopropan-1-amine Yield: 45.1 mg, 46% LCMS m/z = 370 [M + H]⁺ ¹H NMR (400 MHz, DMSO-d₆ + CCl₄) δ: 0.56-0.65 (m, 1H), 0.65-0.74 (m, 1H), 1.01-1.08 (m, 1H), 1.64-1.87 (m, 6H), 1.88- 1.95 (m, 2H), 1.97-2.07 (m, 2H), 2.15-2.25 (m, 1H), 2.57-2.66 (m, 1H), 2.80-2.92 (m, 1H), 3.41-3.51 (m, 2H), 7.49 (s, 1H), 3.90-3.94 (m, 2H), 3.96 (s, 3H), 6.88 (s, 1H), 7.80 (d, 1H), 8.87 (s, 1H). |
| 155 | 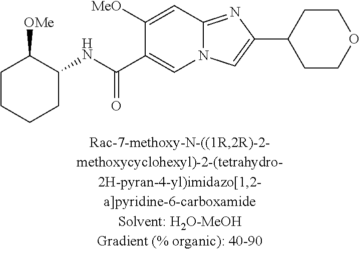 [SMILES: COc1cc2nc(C3CCOCC3)cn2cc1C(=O)N[C@@H]1CCCC[C@H]1OC] | rac-(1R,2R)-2-methoxycyclohexan- 1-amine Yield: 32.7 mg, 32% LCMS m/z = 388 [M + H]⁺ ¹H NMR (400 MHz, DMSO-d₆ + CCl₄) δ: 1.21-1.46 (m, 4H), 1.60-1.64 (m, 1H), 1.65-1.79 (m, 3H), 1.88-1.96 (m, 2H), 2.00- 2.05 (m, 1H), 2.08-2.15 (m, 1H), 2.82-2.92 (m, 1H), 3.16-3.22 (m, 1H), 3.34 (s, 3H), 3.46 (td, 2H), 3.75-3.79 (m, 1H), 3.89-3.97 (m, 2H), 4.00 (s, 3H), 6.92 (s, 1H), 7.50 (s, 1H), 7.87 (d, 1H), 8.90 (s, 1H). |
| 156 | 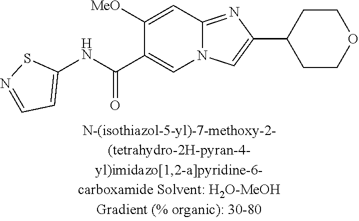 [SMILES: COc1cc2nc(C3CCOCC3)cn2cc1C(=O)Nc1ccns1] | isothiazol-5-amine Yield: 11.8 mg, 12.8% LCMS m/z = 359 [M + H]⁺ ¹H NMR (400 MHz, CDCl₃) δ: 1.75-1.89 (m, 2H), 1.97- 2.00 (m, 2H), 2.94-2.99 (m, 1H), 3.54 (td, 2H), 4.01-4.09 (m, 2H), 4.10 (s, 3H), 6.87-6.93 (m, 1H), 6.98 (s, 1H), 7.29 (s, 1H), 8.23-8.28 (m, 1H), 9.02 (s, 1H), 10.62 (s, 1H). |
| 157 | 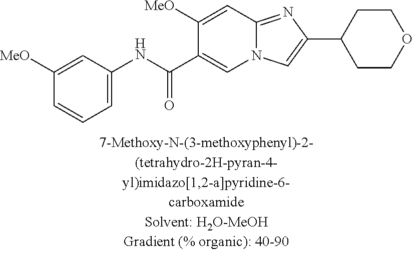 [SMILES: COc1cccc(NC(=O)c2cn3cc(C4CCOCC4)nc3cc2OC)c1] | 3-methoxy aniline Yield: 35.3 mg, 35% LCMS m/z = 382 [M + H]⁺ ¹H NMR (400 MHz, CDCl₃) δ: 1.75-1.90 (m, 2H), 1.97- 2.05 (m, 2H), 2.90-3.03 (m, 1H), 3.49-3.60 (m, 2H), 3.82 (s, 3H), 4.02-4.06 (m, 2H), 4.04-4.08 (m, 3H), 6.69 (dd, 1H), 6.94 (s, 1H), 7.03-7.10 (m, 1H), 7.22-7.29 (m, 2H), 7.38-7.44 (m, 1H), 8.98 (s, 1H), 9.62 (s, 1H). |
| 158 | 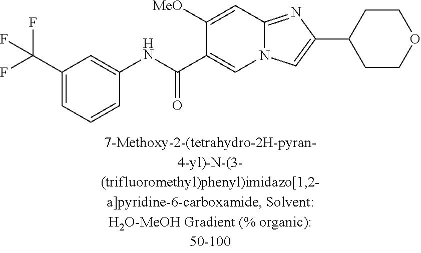 [SMILES: COc1cc2nc(C3CCOCC3)cn2cc1C(=O)Nc1cccc(C(F)(F)F)c1] | 3-trifluoromethylaniline Yield: 32.9 mg, 31% LCMS m/z = 420 [M + H]⁺ ¹H NMR (400 MHz, DMSO-d₆ + CCl₄) δ: 1.70-1.80 (m, 2H), 1.94 (d, 2H), 2.86-2.91 (m, 1H), 3.47 (t, 2H), 3.94 (d, 2H), 4.03 (s, 3H), 6.96 (s, 1H), 7.35 (d, 1H), 7.46- 7.56 (m, 2H), 7.95 (d, 1H), 8.19 (s, 1H), 8.91 (s, 1H), 10.18 (s, 1H). |
| 159 | 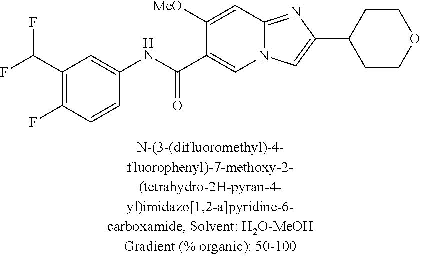 [SMILES: COc1cc2nc(C3CCOCC3)cn2cc1C(=O)Nc1ccc(F)c(C(F)F)c1] | 3-difluoromethyl-4-fluoroaniline Yield: 8.0 mg LCMS m/z = 420 [M + H]⁺ |
| 160 | 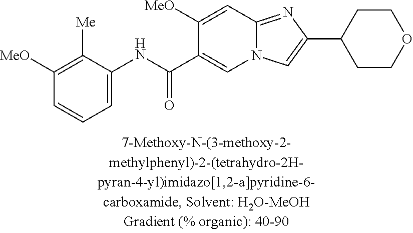 [SMILES: COc1cc2nc(C3CCOCC3)cn2cc1C(=O)Nc1cccc(OC)c1C] | 2-methyl-3-methoxy aniline Yield: 5.5 mg, 5% LCMS m/z = 396 [M + H]⁺ ¹H NMR (400 MHz, DMSO-d₆ + CCl₄) δ: 1.71-1.80 (m, 2H), 1.90-1.98 (m, 2H), 2.18 (s, 3H), 2.87-2.92 (m, 1H), 3.42-3.52 (m, 2H), 3.84 (s, 3H), 3.91-3.98 (m, 2H), 4.10 (s, 3H), 6.73 (d, 1H), 7.02 (s, 1H), 7.13 (t, 1H), 7.57 (s, 1H), 7.62 (d, 1H), 9.10 (s, 1H), 9.67 (s, 1H). |
| 161 | 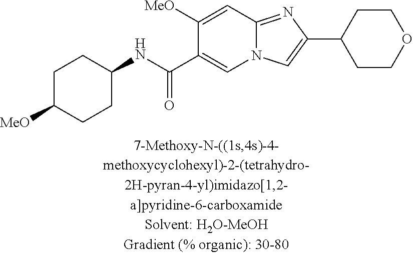 [SMILES: COc1cc2nc(C3CCOCC3)cn2cc1C(=O)N[C@H]1CC[C@@H](OC)CC1] | (1s,4s)-4-methoxycyclohexan-1- amine Yield: 9.6 mg LCMS m/z = 388 [M + H]⁺ ¹H NMR (500 MHz, DMSO-d₆) δ: 1.29-1.39 (m, 4H), 1.65-1.77 (m, 2H), 1.88-1.95 (m, 2H), 1.97-2.02 (m, 4H), 2.82-2.90 (m, 1H), 3.09-3.17 (m, 1H), 3.27 (s, 3H), 3.41-3.49 (m, 2H), 3.77-3.85 (m, 1H), 3.90-3.95 (m, 2H), 3.97 (s, 3H), 6.90 (s, 1H), 7.49 (s, 1H), 7.71 (d, 1H), 8.87 (s, 1H). |
| 162 | 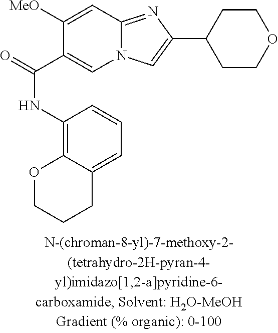 [SMILES: COc1cc2nc(C3CCOCC3)cn2cc1C(=O)Nc1cccc2c1OCCC2] | chroman-8-amine Yield: 32.8 mg, 32% LCMS m/z = 408 [M + H]⁺ ¹H NMR (400 MHz, DMSO-d₆ + CCl₄) δ: 1.67-1.81 (m, 2H), 1.90-1.97 (m, 2H), 2.04-2.09 (m, 2H), 2.78-2.85 (m, 2H), 2.85- 2.91 (m, 1H), 3.47 (t, 2H), 3.91-3.98 (m, 2H), 4.11 (s, 3H), 4.33-4.40 (m, 2H), 6.71-6.82 (m, 2H), 7.01 (s, 1H), 7.58 (s, 1H), 8.26 (d, 1H), 9.14 (s, 1H), 10.39 (s, 1H). |
| 163 | 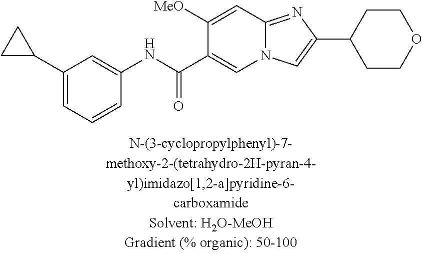 [SMILES: COc1cc2nc(C3CCOCC3)cn2cc1C(=O)Nc1cccc(C2CC2)c1] | 3-cyclopropylaniline Yield: 18.1 mg, 17% LCMS m/z = 392 [M + H]⁺ ¹H NMR (400 MHz, CDCl₃) δ: 0.67-0.75 (m, 2H), 0.91- 1.00 (m, 2H), 1.75-1.88 (m, 2H), 1.88-1.96 (m, 1H), 1.97-2.05 (m, 2H), 2.94-2.99 (m, 1H), 3.55 (td, 2H), 4.02-4.06 (m, 2H), 4.06 (s, 3H), 6.84 (d, 1H), 6.94 (s, 1H), 7.20-7.28 (m, 2H), 7.35 (d, 1H), 7.40 (d, 1H), 8.98 (s, 1H), 9.55 (s, 1H). |
| 164 | 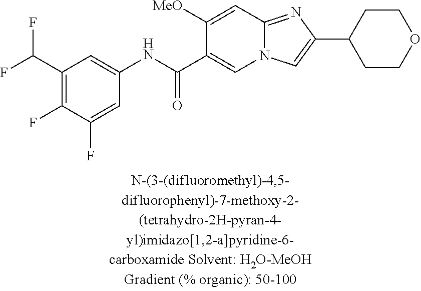 [SMILES: COc1cc2nc(C3CCOCC3)cn2cc1C(=O)Nc1cc(F)c(F)c(C(F)F)c1] | 3-(difluoromethyl)-4,5- difluoroaniline Yield: 20.7 mg, 21% LCMS m/z = 438 [M + H]⁺ ¹H NMR (400 MHz, DMSO-d₆ + CCl₄) δ: 1.69-1.80 (m, 2H), 1.90-1.97 (m, 2H), 2.86-2.91 (m, 1H), 3.42-3.52 (m, 2H), 3.91- 3.98 (m, 2H), 4.02 (s, 3H), 6.91-7.23 (m, 2H), 7.53 (s, 1H), 7.75-7.80 (8.89 (s, 1H), m, 1H), 8.06-8.11 (m, 1H), 10.21 (s, 1H). |
| 165 | 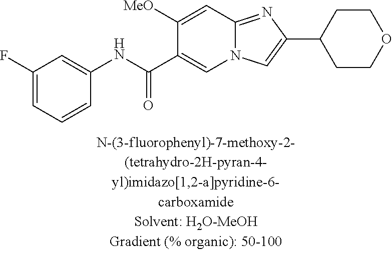 [SMILES: COc1cc2nc(C3CCOCC3)cn2cc1C(=O)Nc1cccc(F)c1] | 3-fluoroaniline Yield: 32 mg LCMS m/z = 370 [M + H]⁺ ¹H NMR (400 MHz, DMSO-d₆ + CCl₄) δ: 1.67-1.81 (m, 2H), 1.89-1.97 (m, 2H), 2.86-2.91 (m, 1H), 3.42-3.52 (m, 2H), 3.90- 3.98 (m, 2H), 4.03 (s, 3H), 6.80 (t, 1H), 6.96 (s, 1H), 7.25-7.35 (m, 1H), 7.43 (d, 1H), 7.53 (s, 1H), 7.73 (d, 1H), 8.92 (s, 1H), 10.05 (s, 1H). |
| 166 | 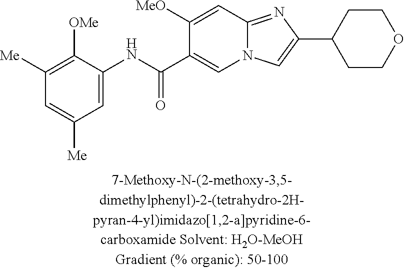 [SMILES: COc1cc2nc(C3CCOCC3)cn2cc1C(=O)Nc1cc(C)cc(C)c1OC] | 2-methoxy-3,5-dimethylaniline Yield: 7.5 mg, 7% LCMS m/z = 410 [M + H]⁺ ¹H NMR (500 MHz, DMSO-d₆) δ: 1.74 (qd, 2H), 1.90- 1.97 (m, 2H), 2.27 (s, 3H), 2.30 (s, 3H), 2.85-2.94 (m, 1H), 3.47 (td, 2H), 3.78 (s, 3H), 3.91-3.98 (m, 2H), 4.15 (s, 3H), 6.69 (s, 1H), 7.05 (s, 1H), 7.60 (s, 1H), 8.13 (d, 1H), 9.13 (s, 1H), 10.34 (s, 1H). |
| 167 | 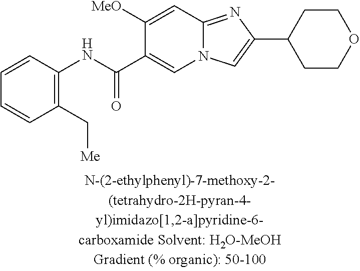 [SMILES: CCc1ccccc1NC(=O)c1cn2cc(C3CCOCC3)nc2cc1OC] | 2-ethylaniline Yield: 14.8 mg, 15% LCMS m/z = 380 [M + H]⁺ ¹H NMR (400 MHz, CDCl₃)δ: 1.28 (t, 3H), 1.83 (qd, 2H), 1.98-2.06 (m, 2H), 2.68 (q, 2H), 2.90-3.03 (m, 1H), 3.50-3.60 (m, 2H), 4.01-4.09 (m, 5H), 6.97 (s, 1H), 7.12 (t, 1H), 7.19-7.27 (m, 2H), 7.28 (s, 1H), 8.16 (d, 1H), 9.03 (s, 1H), 9.63 (s, 1H). |
| 168 | 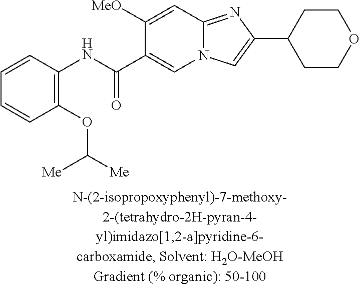 [SMILES: COc1cc2nc(C3CCOCC3)cn2cc1C(=O)Nc1ccccc1OC(C)C] | 2-isopropoxy aniline Yield: 6.0 mg, 6% LCMS m/z = 410 [M + H]⁺ |
| 169 | 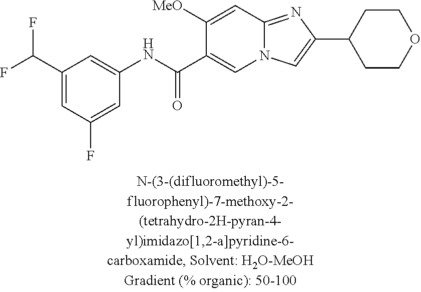 [SMILES: COc1cc2nc(C3CCOCC3)cn2cc1C(=O)Nc1cc(F)cc(C(F)F)c1] | 3-(difluoromethyl)-5-fluoroaniline hydrochloride Yield: 16.2 mg, 17% LCMS m/z = 420 [M + H]⁺ ¹H NMR (400 MHz, DMSO-d₆ + CCl₄) δ: 1.67-1.80 (m, 2H), 1.90-1.98 (m, 2H), 2.83-2.95 (m, 1H), 3.42-3.52 (m, 2H), 3.91- 3.98 (m, 2H), 4.03 (s, 3H), 6.71-7.04 (m, 3H), 7.53 (s, 1H), 7.74 (s, 1H), 7.85 (d, 1H), 8.90 (s, 1H), 10.23 (s, 1H). |
| 170 | 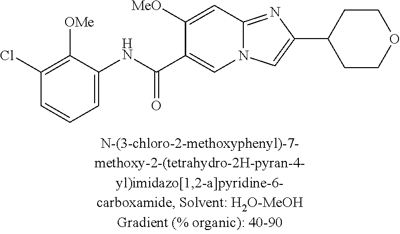 [SMILES: COc1cc2nc(C3CCOCC3)cn2cc1C(=O)Nc1cccc(Cl)c1OC] | 3-chloro-2-methoxy aniline Yield: 13.3 mg, 12% LCMS m/z = 416 [M + H]⁺ ¹H NMR (400 MHz, CDCl₃) δ: 1.82 (qd, 2H), 1.98-2.06 (m, 2H), 2.95-3.00 (m, 1H), 3.50- 3.60 (m, 2H), 3.94 (s, 3H), 4.02-4.09 (m, 2H), 4.11 (s, 3H), 6.97 (s, 1H), 7.03-7.13 (m, 2H), 7.28 (s, 1H), 8.43-8.50 (m, 1H), 9.00 (s, 1H), 10.49 (s, 1H). |
| 171 | 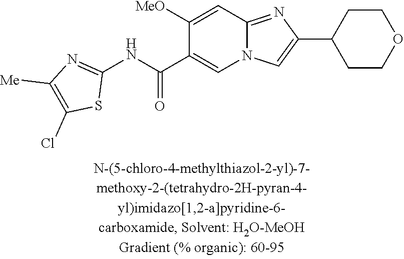 [SMILES: COc1cc2nc(C3CCOCC3)cn2cc1C(=O)Nc1nc(C)c(Cl)s1] | 5-chloro-4-methylthiazol-2-amine Yield: 16.5 mg, 16% LCMS m/z = 407 [M + H]⁺ ¹H NMR (500 MHz, DMSO-d₆) δ: 1.67-1.79 (m, 2H), 1.89-1.96 (m, 2H), 2.28 (s, 3H), 2.88 (tt, 1H), 3.42-3.51 (m, 2H), 3.90-3.97 (m, 2H), 4.05 (s, 3H), 6.99 (s, 1H), 7.54 (s, 1H), 9.05 (s, 1H), 11.41 (s, 1H). |
| 172 | 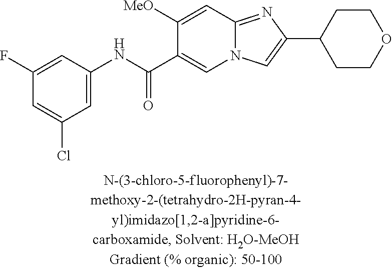 [SMILES: COc1cc2nc(C3CCOCC3)cn2cc1C(=O)Nc1cc(F)cc(Cl)c1] | 3-chloro-5-fluoroaniline Yield: 14.1 mg, 16% LCMS m/z = 404 [M + H]⁺ ¹H NMR (400 MHz, DMSO-d₆ + CCl₄) δ: 1.67-1.79 (m, 2H), 1.90-1.97 (m, 2H), 2.81-2.93 (m, 1H), 3.42-3.52 (m, 2H), 3.91- 3.98 (m, 2H), 4.02 (s, 3H), 6.84-6.91 (m, 1H), 6.95 (s, 1H), 7.53 (s, 1H), 7.62-7.68 (m, 2H), 8.90 (s, 1H), 10.16 (s, 1H). |
| 173 | 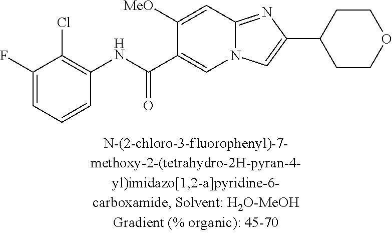 [SMILES: COc1cc2nc(C3CCOCC3)cn2cc1C(=O)Nc1cccc(F)c1Cl] | 2-chloro-3-fluoroaniline Yield: 9.4 mg, 9% LCMS m/z = 404 [M + H]⁺ |
| 174 | 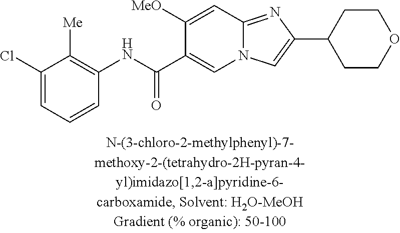 [SMILES: COc1cc2nc(C3CCOCC3)cn2cc1C(=O)Nc1cccc(Cl)c1C] | 3-chloro-3-methylaniline Yield: 8.4 mg, 8% LCMS m/z = 400 [M + H]⁺ ¹H NMR (500 MHz, DMSO-d₆) δ: 1.68-1.80 (m, 2H), 1.90-1.97 (m, 2H), 2.39 (s, 3H), 2.89 (tt, 1H), 3.43-3.51 (m, 2H), 3.91-3.98 (m, 2H), 4.09 (s, 3H), 7.02 (s, 1H), 7.15-7.23 (m, 2H), 7.57 (s, 1H), 7.84-7.91 (m, 1H), 9.08 (s, 1H), 9.78 (s, 1H). |
| 175 | 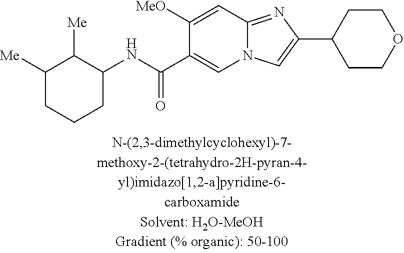 [SMILES: COc1cc2nc(C3CCOCC3)cn2cc1C(=O)NC1CCCC(C)C1C] | 2,3-dimethylcyclohexylamine Yield: 8.8 mg, 9% LCMS m/z = 386 [M + H]⁺ ¹H NMR (400 MHz, DMSO-d₆ + CCl₄) δ: 0.89-1.01 (m, 6H), 1.02-1.08 (m, 1H), 1.19-1.27 (m, 1H), 1.32-1.57 (m, 2H), 1.66- 1.76 (m, 4H), 1.88-1.96 (m, 3H), 2.80-2.93 (m, 1H), 3.41-3.51 (m, 2H), 3.91-3.50 (m, 1H), 3.90-3.96 (m, 2H), 3.96-4.06 (m, 3H), 6.89- 6.98 (m, 1H), 7.47-7.53 (m, 1H), 7.54-7.80 (m, 1H), 8.84-8.92 (m, 1H). |
| 176 | 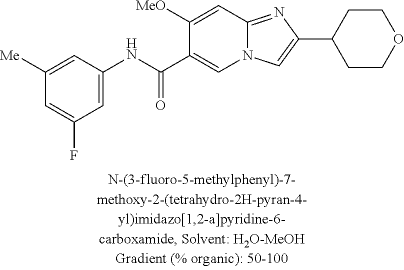 [SMILES: COc1cc2nc(C3CCOCC3)cn2cc1C(=O)Nc1cc(C)cc(F)c1] | 3-fluoro-5-methylaniline Yield: 29.6 mg, 32% LCMS m/z = 384 [M + H]⁺ ¹H NMR (400 MHz, DMSO-d₆ + CCl₄) δ: 1.73 (qd, 2H), 1.93 (d, 2H), 2.37 (s, 3H), 2.80-2.96 (m, 1H), 3.42-3.52 (m, 2H), 3.91- 3.98 (m, 2H), 4.04 (s, 3H), 6.63 (d, 1H), 6.96 (s, 1H), 7.23 (s, 1H), 7.48- 7.55 (m, 2H), 8.91 (s, 1H), 9.95 (s, 1H). |
| 177 | 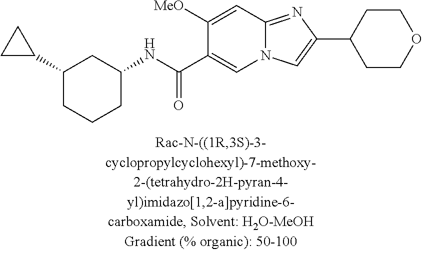 [SMILES: COc1cc2nc(C3CCOCC3)cn2cc1C(=O)N[C@@H]1CCC[C@H](C2CC2)C1] | rel-(1R,3S)-3- cyclopropylcyclohexan-1-amine hydrochloride Yield: 5.6 mg, 6% LCMS m/z = 398 [M + H]⁺ |
| 178 | 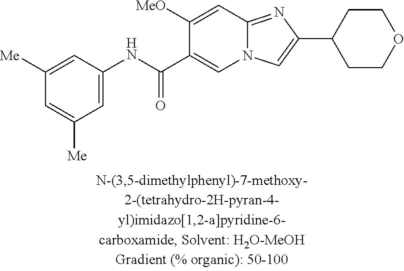 [SMILES: COc1cc2nc(C3CCOCC3)cn2cc1C(=O)Nc1cc(C)cc(C)c1] | 3,5-dimethylaniline Yield: 45.7 mg, 41% LCMS m/z = 380 [M + H]⁺ ¹H NMR (400 MHz, DMSO-d₆ + CCl₄) δ: 1.69-1.80 (m, 2H), 1.94 (d, 2H), 2.32 (s, 6H), 2.83- 2.93 (m, 1H), 3.47 (t, 2H), 3.94 (d, 2H), 4.06 (s, 3H), 6.70 (s, 1H), 6.96 (s, 1H), 7.30 (s, 2H), 7.54 (s, 1H), 8.92 (s, 1H), 9.72 (s, 1H). |
| 179 | 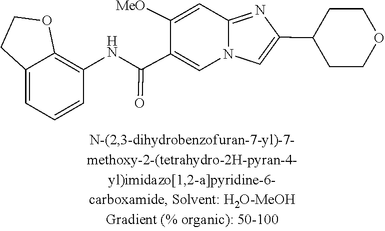 [SMILES: COc1cc2nc(C3CCOCC3)cn2cc1C(=O)Nc1cccc2c1OCC2] | 2,3-dihydrobenzofuran-7-amine Yield: 27.4 mg, 28% LCMS m/z = 394 [M + H]⁺ ¹H NMR (400 MHz, DMSO-d₆ + CCl₄) δ: 1.70-1.77 (m, 2H), 1.90-1.98 (m, 2H), 2.87-2.91 (m, 1H), 3.30 (t, 2H), 3.42-3.52 (m, 2H), 3.91-3.98 (m, 2H), 4.11 (s, 3H), 4.69 (t, 2H), 6.80 (t, 1H), 6.93 (d, 1H), 7.02 (s, 1H), 7.58 (s, 1H), 8.12 (d, 1H), 9.14 (s, 1H), 9.95 (s, 1H). |
| 180 | 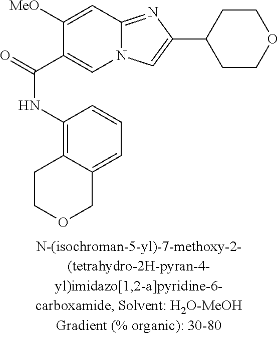 [SMILES: COc1cc2nc(C3CCOCC3)cn2cc1C(=O)Nc1cccc2c1CCOC2] | isochroman-5-amine hydrochloride Yield: 6.8 mg LCMS m/z = 408 [M + H]⁺ ¹H NMR (500 MHz, DMSO-d₆) δ: 1.73 (qd, 2H), 1.90- 1.97 (m, 2H), 2.75 (t, 2H), 2.85-2.94 (m, 1H), 3.43-3.51 (m, 2H), 3.91- 3.96 (m, 2H), 3.98 (t, 2H), 4.10 (s, 3H), 4.70 (s, 2H), 6.81 (d, 1H), 7.01 (s, 1H), 7.16 (t, 1H), 7.57 (s, 1H), 7.92 (d, 1H), 9.10 (s, 1H), 9.66 (s, 1H). |
| 181 | 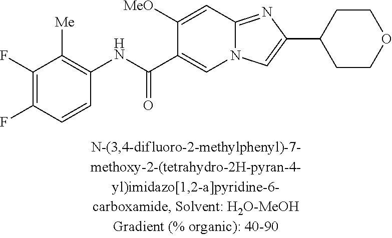 [SMILES: COc1cc2nc(C3CCOCC3)cn2cc1C(=O)Nc1ccc(F)c(F)c1C] | 3,4-difluoro-2-methylaniline Yield: 5.7 mg, 5% LCMS m/z = 402 [M + H]⁺ |
| 182 | 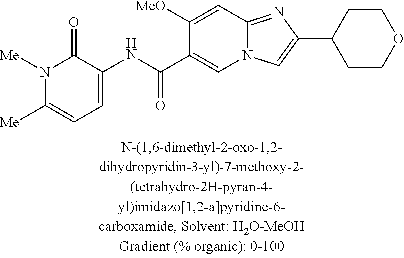 [SMILES: COc1cc2nc(C3CCOCC3)cn2cc1C(=O)Nc1ccc(C)n(C)c1=O] | 3-amino-1,6-dimethylpyridin-2(1H)- one Yield: 94.4 mg; 94% LCMS m/z = 397 [M + H] ¹H NMR (400 MHz, DMSO-d₆) δ: 1.67-1.79 (m, 2H), 1.89-1.97 (m, 2H), 2.39 (s, 3H), 2.82-2.96 (m, 1H), 3.42-3.52 (m, 2H), 3.57 (s, 3H), 3.91-3.98 (m, 2H), 4.14 (s, 3H), 6.11 (d, 1H), 6.99 (s, 1H), 7.57 (s, 1H), 8.31 (d, 1H), 9.11 (s, 1H), 10.72 (s, 1H). |
| 183 | 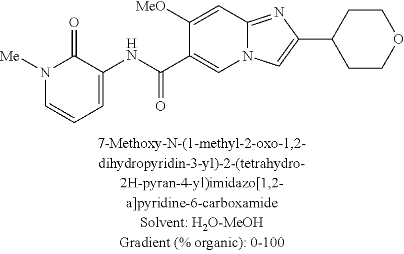 [SMILES: COc1cc2nc(C3CCOCC3)cn2cc1C(=O)Nc1cccn(C)c1=O] | 1-methyl-2-oxo-1,2-dihydropyridin- 3-amine Yield: 75.2 mg; 75% LCMS m/z = 383 [M + H]⁺ ¹H NMR (400 MHz, DMSO-d₆) δ: 1.67-1.79 (m, 2H), 1.89-1.97 (m, 2H), 2.86-2.91 (m, 1H), 3.41-3.52 (m, 2H), 3.58 (s, 3H), 3.91-3.98 (m, 2H), 4.14 (s, 3H), 6.22 (t, 1H), 7.00 (s, 1H), 7.32 (dd, 1H), 7.58 (s, 1H), 8.42 (dd, 1H), 9.13 (s, 1H), 10.82 (s, 1H). |
| 184 | 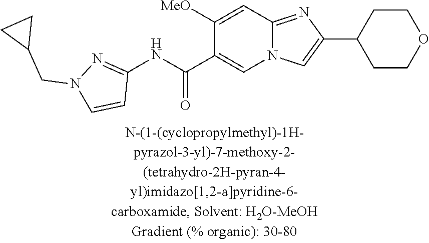 [SMILES: COc1cc2nc(C3CCOCC3)cn2cc1C(=O)Nc1ccn(CC2CC2)n1] | 1-(cyclopropylmethyl)-1H-pyrazol- 3-amine Yield: 8.4 mg; 8.4% LCMS m/z = 396 [M + H]⁺ |
| 185 | 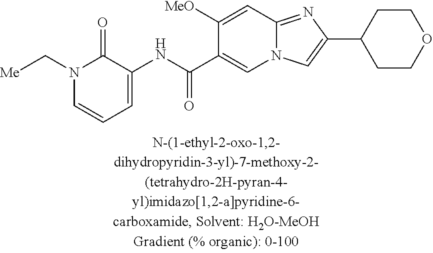 [SMILES: CCn1cccc(NC(=O)c2cn3cc(C4CCOCC4)nc3cc2OC)c1=O] | 1-ethyl-2-oxo-1,2-dihydropyridin-3- amine Yield: 48 mg; 48% LCMS m/z = 397 [M + H]⁺ ¹H NMR (400 MHz, DMSO-d₆ + CCl₄) δ: 1.30-1.38 (m, 3H), 1.69-1.77 (m, 2H), 1.89-1.97 (m, 2H), 2.86-2.91 (m, 1H), 3.42- 3.52 (m, 2H), 4.15 (s, 3H), 3.90-3.98 (m, 2H), 3.99-4.09 (m, 2H), 6.24 (t, 1H), 7.00 (s, 1H), 7.30 (d, 1H), 7.57 (s, 1H), 8.42 (d, 1H), 9.13 (s, 1H), 10.84 (s, 1H). |
| 186 | 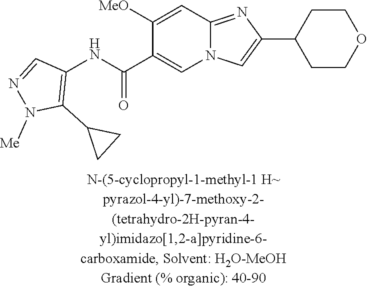 [SMILES: COc1cc2nc(C3CCOCC3)cn2cc1C(=O)Nc1cnn(C)c1C1CC1] | 5-cyclopropyl-1-methyl-1H-pyrazol- 4-amine Yield: 46 mg; 46% LCMS m/z = 397 [M + H]⁺ ¹H NMR (400 MHz, DMSO-d₆) δ: 0.71-0.78 (m, 2H), 1.04-1.13 (m, 2H), 1.69-1.80 (m, 3H), 1.90-1.98 (m, 2H), 2.84-2.91 (m, 1H), 3.42-3.52 (m, 2H), 3.85 (s, 3H), 3.90-3.98 (m, 2H), 4.11 (s, 3H), 7.03 (s, 1H), 7.57 (s, 1H), 7.77 (s, 1H), 9.10 (s, 1H), 9.51 (s, 1H). |
| 187 | 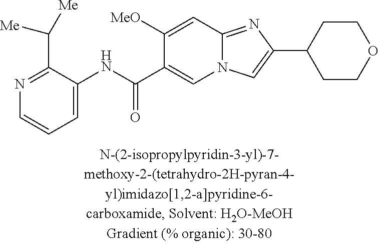 [SMILES: COc1cc2nc(C3CCOCC3)cn2cc1C(=O)Nc1cccnc1C(C)C] | 2-isopropylpyridin-3-amine Yield: 38 mg; 38% LCMS m/z = 395 [M + H]⁺ ¹H NMR (400 MHz, DMSO-d₆ + CCl₄) δ: 1.31 (d, 6H), 1.67-1.81 (m, 2H), 1.90-1.98 (m, 2H), 2.84-2.95 (m, 1H), 3.28 (hept, 1H), 3.42-3.52 (m, 2H), 3.91-3.98 (m, 2H), 4.10 (s, 3H), 7.05 (s, 1H), 7.19 (dd, 1H), 7.59 (s, 1H), 8.27-8.33 (m, 2H), 9.12 (s, 1H), 9.82 (s, 1H). |
| 188 | 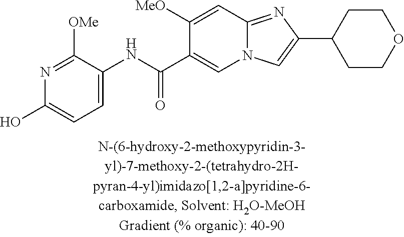 [SMILES: COc1cc2nc(C3CCOCC3)cn2cc1C(=O)Nc1ccc(O)nc1OC] | 6-hydroxy-2-methoxypyridin-3- amine Yield: 9.5 mg; 9.5% LCMS m/z = 399 [M + H]⁺ |
| 189 | 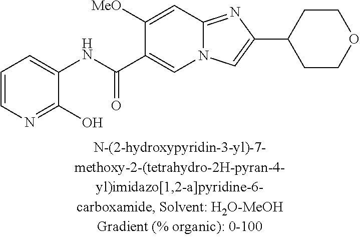 [SMILES: COc1cc2nc(C3CCOCC3)cn2cc1C(=O)Nc1cccnc1O] | 2-hydroxypyridin-3-amine Yield: 79 mg; 79% LCMS m/z = 369 [M + H]⁺ ¹H NMR (400 MHz, DMSO-d₆) δ: 1.69-1.81 (m, 2H), 1.90-1.98 (m, 2H), 2.83-2.96 (m, 1H), 3.47 (t, 2H), 3.91-3.98 (m, 2H), 4.13 (s, 3H), 6.19 (t, 1H), 6.98-7.03 (m, 2H), 7.58 (s, 1H), 8.43 (d, 1H), 9.13 (s, 1H), 10.71 (s, 1H), 11.91 (s, 1H). |
| 190 | 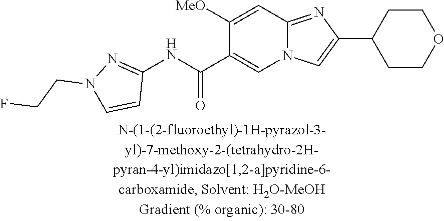 [SMILES: COc1cc2nc(C3CCOCC3)cn2cc1C(=O)Nc1ccn(CCF)n1] | 1-(2-fluoroethyl)-1H-pyrazol-3- amine Yield: 14 mg; 14% LCMS m/z = 388 [M + H]⁺ ¹H NMR (400 MHz, CDCl₃) δ: 1.72-1.90 (m, 2H), 1.96- 2.04 (m, 2H), 2.96 (tt, 1H), 3.49-3.59 (m, 2H), 4.01-4.09 (m, 5H), 4.27 (t, 1H), 4.34 (t, 1H), 4.66 (t, 1H), 4.78 (t, 1H), 6.81 (d, 1H), 6.93 (s, 1H), 7.26 (s, 1H), 7.39 (d, 1H), 8.98 (s, 1H), 9.95 (s, 1H). |
| 191 | 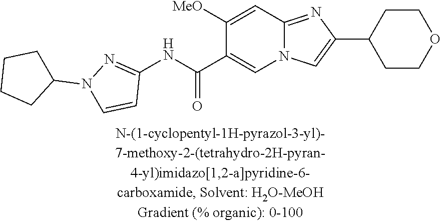 [SMILES: COc1cc2nc(C3CCOCC3)cn2cc1C(=O)Nc1ccn(C2CCCC2)n1] | 1-cyclopentyl-1H-pyrazol-3-amine Yield: 53 mg; 53% LCMS m/z = 410 [M + H]⁺ ¹H NMR (400 MHz, DMSO-d₆ + CCl₄) δ: 1.66-1.80 (m, 4H), 1.82-1.93 (m, 3H), 1.93-2.03 (m, 3H), 2.05-2.13 (m, 2H), 2.86- 2.90 (m, 1H), 3.41-3.52 (m, 2H), 3.90-3.98 (m, 2H), 4.10 (s, 3H), 4.56-4.58 (m, 1H), 6.61 (d, 1H), 6.99 (s, 1H), 7.48 (d, 1H), 7.55 (s, 1H), 9.04 (s, 1H), 9.98 (s, 1H). |
| 192 | 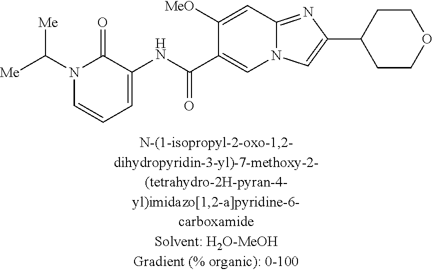 [SMILES: COc1cc2nc(C3CCOCC3)cn2cc1C(=O)Nc1cccn(C(C)C)c1=O] | 1-isopropyl-2-oxo-1,2- dihydropyri din-3-amine Yield: 25 mg; 25% LCMS m/z = 411 [M + H]⁺ ¹H NMR (400 MHz, DMSO-d₆) δ: 1.40 (d, 6H), 1.69-1.80 (m, 2H), 1.89-1.98 (m, 2H), 2.86- 2.91 (m, 1H), 3.42-3.52 (m, 2H), 3.90-3.98 (m, 2H), 4.15 (s, 3H), 5.20 (hept, 1H), 6.29 (t, 1H), 7.00 (s, 1H), 7.31 (dd, 1H), 7.57 (s, 1H), 8.40 (dd, 1H), 9.12 (s, 1H), 10.86 (s, 1H). |
| 193 | 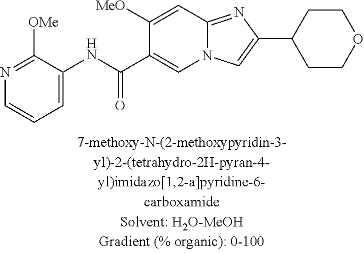 [SMILES: COc1cc2nc(C3CCOCC3)cn2cc1C(=O)Nc1cccnc1OC] | 2-methoxypyridine-3-amine Yield: 56 mg; 56% LCMS m/z = 383 [M + H]⁺ ¹H NMR (400 MHz, DMSO-d₆) δ: 1.66-1.81 (m, 2H), 1.89-1.97 (m, 2H), 2.84-2.94 (m, 1H), 3.42-3.52 (m, 2H), 3.91-3.98 (m, 2H), 4.06 (s, 3H), 4.14 (s, 3H), 6.94 (dd, 1H), 7.04 (s, 1H), 7.59 (s, 1H), 7.81 (dd, 1H), 8.71 (dd, 1H), 9.16 (s, 1H), 10.41 (s, 1H). |
| 194 | 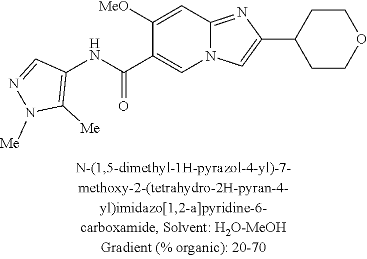 [SMILES: COc1cc2nc(C3CCOCC3)cn2cc1C(=O)Nc1cnn(C)c1C] | 1,5-dimethyl-1H-pyrazol-4-amine Yield: 48 mg; 48% LCMS m/z = 370 [M + H]⁺ ¹H NMR (400 MHz, DMSO-d₆ + CCl₄) δ: 1.67-1.80 (m, 2H), 1.89-1.97 (m, 2H), 2.24 (s, 3H), 2.86-2.90 (m, 1H), 3.42-3.52 (m, 2H), 3.76 (s, 3H), 3.90-3.98 (m, 2H), 4.04 (s, 3H), 6.96 (s, 1H), 7.53 (d, 2H), 8.97 (s, 1H), 9.27-9.31 (m, 1H). |
| 195 | 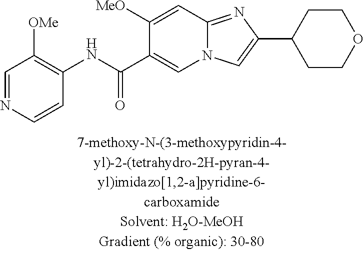 [SMILES: COc1cnccc1NC(=O)c1cn2cc(C3CCOCC3)nc2cc1OC] | 3-methoxypyridine-4-amine Yield: 6.8 mg; 6.8% LCMS m/z = 383, [M + H]⁺ ¹H NMR (500 MHz, DMSO-d₆ + CCl₄) δ: 1.71-1.77 (m, 2H), 1.91-1.97 (m, 2H), 2.88-2.92 (m, 1H), 3.43-3.51 (m, 2H), 3.92- 3.98 (m, 2H), 4.09 (s, 3H), 4.14 (s, 3H), 7.06 (s, 1H), 7.55 (s, 1H), 7.60 (s, 1H), 8.11-8.15 (m, 1H), 8.27 (s, 1H), 8.38 (d, 1H), 9.20 (s, 1H), 10.61 (s, 1H). |
| 196 | 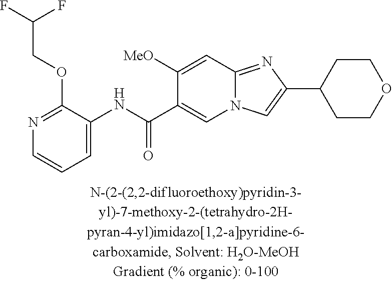 [SMILES: COc1cc2nc(C3CCOCC3)cn2cc1C(=O)Nc1cccnc1OCC(F)F] | 2-(2,2-difluoroethoxy)pyridin-3- amine Yield: 60 mg; 60% LCMS m/z = 433 [M + H]⁺ ¹H NMR (400 MHz, DMSO-d₆) δ: 1.70-1.80 (m, 2H), 1.90-1.98 (m, 2H), 2.87-2.91 (m, 1H), 3.42-3.52 (m, 2H), 3.90-3.98 (m, 2H), 4.12 (s, 3H), 4.62-4.75 (m, 2H), 6.40 (t, 1H), 7.00-7.07 (m, 2H), 7.59 (s, 1H), 7.83 (dd, 1H), 8.83 (d, 1H), 9.18 (s, 1H), 10.31 (s, 1H). |
| 197 | 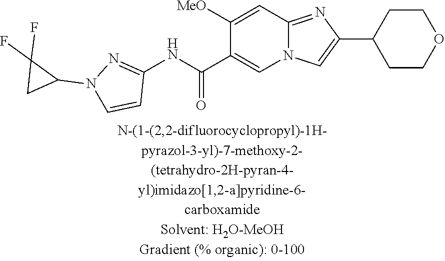 [SMILES: COc1cc2nc(C3CCOCC3)cn2cc1C(=O)Nc1ccn(C2CC2(F)F)n1] | 1-(2,2-difluorocy clopropyl)-1H- pyrazol-3-amine Yield: 54 mg; 54% LCMS m/z = 418 [M + H]⁺ ¹H NMR (400 MHz, DMSO-d₆) δ: 1.67-1.79 (m, 2H), 1.89-1.97 (m, 2H), 2.12-2.19 (m, 1H), 2.27-2.40 (m, 1H), 2.83-2.93 (m, 1H), 3.42-3.52 (m, 2H), 3.90- 3.98 (m, 2H), 4.09 (s, 3H), 4.23-4.31 (m, 1H), 6.74 (d, 1H), 6.99 (s, 1H), 7.55 (s, 1H), 7.67 (d, 1H), 9.03 (s, 1H), 10.12 (s, 1H). |
| 198 | 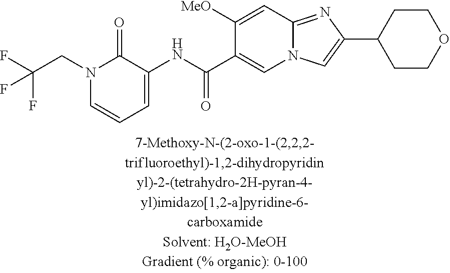 [SMILES: COc1cc2nc(C3CCOCC3)cn2cc1C(=O)Nc1cccn(CC(F)(F)F)c1=O] | 3-amino-1-(2,2,2- trifluoroethyl)pyridin-2(1H)-one Yield: 75 mg; 54% LCMS m/z = 451 [M + H]⁺ ¹H NMR (400 MHz, DMSO-d₆) δ: 1.66-1.80 (m, 2H), 1.89-1.97 (m, 2H), 2.86-2.91 (m, 1H), 3.42-3.52 (m, 2H), 3.90-3.98 (m, 2H), 4.14 (s, 3H), 4.91 (q, 2H), 6.32 (t, 1H), 7.01 (s, 1H), 7.34 (d, 1H), 7.58 (s, 1H), 8.49 (dd, 1H), 9.14 (s, 1H), 10.82 (s, 1H). |
| 199 | 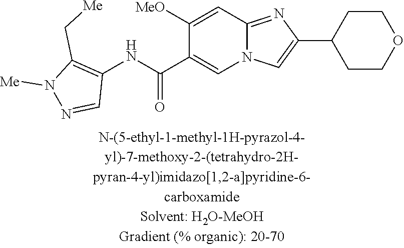 [SMILES: CCc1c(NC(=O)c2cn3cc(C4CCOCC4)nc3cc2OC)cnn1C] | 5-ethyl-1-methyl-1H-pyrazol-4- amine Yield: 50 mg; 50% LCMS m/z = 384 [M + H]⁺ ¹H NMR (400 MHz, DMSO-d₆ + CCl₄) δ: 1.21 (t, 3H), 1.67-1.79 (m, 2H), 1.90-1.97 (m, 2H), 2.70 (q, 2H), 2.83-2.91 (m, 1H), 3.41-3.52 (m, 2H), 3.79 (s, 3H), 3.90-3.98 (m, 2H), 4.04 (s, 3H), 6.97 (s, 1H), 7.54 (s, 1H), 7.56 (s, 1H), 8.99 (s, 1H), 9.29 (s, 1H). |
| 200 | 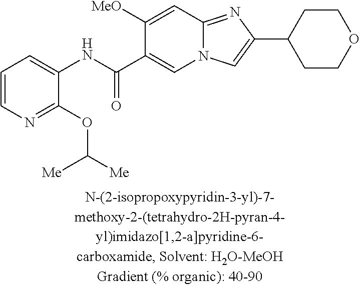 [SMILES: COc1cc2nc(C3CCOCC3)cn2cc1C(=O)Nc1cccnc1OC(C)C] | 2-isopropoxypyridin-3-amine Yield: 5 mg; 5% LCMS m/z = 411 [M + H]⁺ |
| 201 | 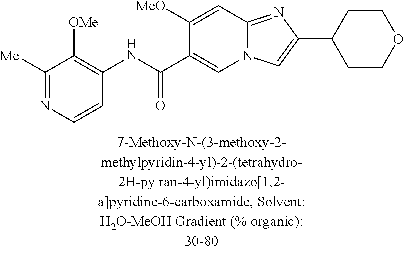 [SMILES: COc1cc2nc(C3CCOCC3)cn2cc1C(=O)Nc1ccnc(C)c1OC] | 3-methoxy-2-methylpyridin-4-amine Yield: 6 mg; 6% LCMS m/z = 397 [M + H]⁺ |
| 202 | 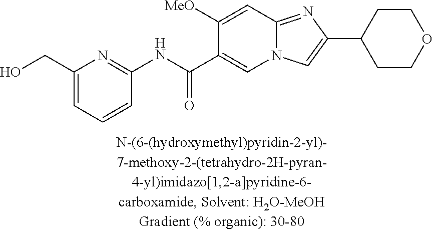 [SMILES: COc1cc2nc(C3CCOCC3)cn2cc1C(=O)Nc1cccc(CO)n1] | 6-(hydroxymethyl)pyridin-2-amine Yield: 5.4 mg; 5.4% LCMS m/z = 383 [M + H]⁺ |
| 203 | 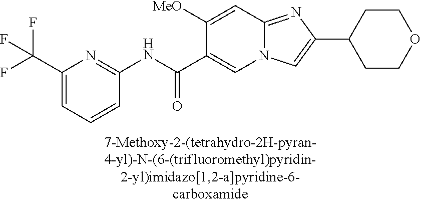 [SMILES: COc1cc2nc(C3CCOCC3)cn2cc1C(=O)Nc1cccc(C(F)(F)F)n1] | 6-(trifluoromethyl)pyridin-2-amine LCMS m/z = 421 [M + H]⁺ |

### Example 204: 7-Methoxy-N-(pyrido[3,2-d]pyrimidin-4-yl)-2-(tetrahydro-2H-pyran-4-yl)imidazo[1,2-a]pyridine-6-carboxamide

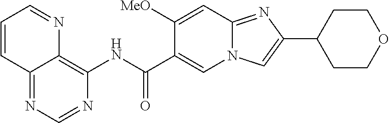 [SMILES: COc1cc2nc(C3CCOCC3)cn2cc1C(=O)Nc1ncnc2cccnc12]

To a mixture of 7-methoxy-2-(tetrahydro-2H-pyran-4-yl)imidazo[1,2-a]pyridine-6-carboxylic acid (Preparation 75, 68.3 mg, 0.25 mmol) and N-methyl-imidazole (60.9 mg, 0.74 mmol) in MeCN (2 mL,) in an 8 mL vial was added MsCl (28.3 mg, 0.25 mmol) and the mixture stirred for 30 min at 50° C. To the resulting mixture was added pyrido[3,2-d]pyrimidin-4-amine (36.1 mg, 0.25 mmol) and the vial sealed and stirred at 100° C. for 6 h. The reaction mixture was evaporated to dryness in vacuo and the residue dissolved in DMSO (0.5 mL) and filtered. The filtrate was purified by prep. HPLC (Waters SunFire C18 19*100 5 μm column; H₂O-MeCN; % organic 30-80) to afford 7-methoxy-N-(pyrido[3,2-d]pyrimidin-4-yl)-2-(tetrahydro-2H-pyran-4-yl)imidazo[1,2-a]pyridine-6-carboxamide (4 mg, 4%). LCMS m/z=405 [M+H]⁺;

### Example 205: N-chroman-8-yl-8-methoxy-2-tetrahydropyran-4-yl-imidazo[1,2-a]pyrazine-6-carboxamide trifluoroacetate

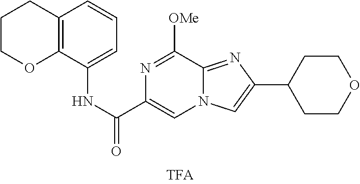 [SMILES: COc1nc(C(=O)Nc2cccc3c2OCCC3)cn2cc(C3CCOCC3)nc12]

To a mixture of 8-methoxy-2-(tetrahydro-2H-pyran-4-yl)imidazo[1,2-a]pyrazine-6-carboxylic acid (Preparation 74A and B, 82 mg, 0.296 mmol), chroman-8-amine (88.4 mg, 0.592 mmol) and DIPEA (191 mg, 1.48 mmol) in EtOAc (4 mL) in a 2-dram vial was added T3P® (50 wt. % in EtOAc)® (529 μL, 0.888 mmol, 50% purity) at rt. The vial was capped and stirred at 22° C. overnight. The cooled reaction was partitioned between EtOAc and H₂O and the organic phase washed with brine, dried (MgSO₄) and evaporated to dryness in vacuo. The residue was purified by prep HPLC (SunFire C18 column, 60 mL/min flow rate, MeCN/H₂O/0.1% TFA; Gradient (% organic): 10-70) to afford N-chroman-8-yl-8-methoxy-2-tetrahydropyran-4-yl-imidazo[1,2-a]pyrazine-6-carboxamide trifluoroacetate as a white solid (7.8 mg, 6.5%). LCMS m/z=393 [M+H]⁺; ¹H NMR (500 MHz, CDCl₃) δ: 1.79-1.97 (m, 3H), 2.06-2.16 (m, 5H), 2.86 (t, 3H), 3.02-3.32 (m, 11H), 3.62 (td, 2H), 4.09-4.19 (m, 2H), 4.24-4.32 (m, 3H), 4.32-4.39 (m, 2H), 6.85-6.97 (m, 2H), 7.57 (s, 1H), 8.30-8.39 (m, 1H), 8.72 (s, 1H), 10.22 (s, 1H).

### Examples 206 and 207: N-[6-(difluoromethyl)-2-pyridyl]-8-methoxy-2-tetrahydropyran-4-ylimidazo[1,2-a]pyrazine-6-carboxamide trifluoroacetate

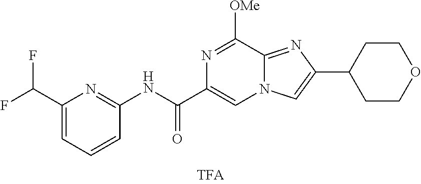 [SMILES: COc1nc(C(=O)Nc2cccc(C(F)F)n2)cn2cc(C3CCOCC3)nc12]

and

### N-[6-(difluoromethyl)-2-pyridyl]-8-ethoxy-2-tetrahydropyran-4-ylimidazo[1,2-a]pyrazine-6-carboxamide trifluoroacetate

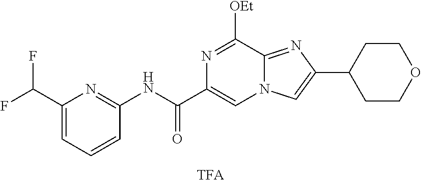 [SMILES: CCOc1nc(C(=O)Nc2cccc(C(F)F)n2)cn2cc(C3CCOCC3)nc12]

Part A. To a mixture of 8-methoxy-2-(tetrahydro-2H-pyran-4-yl)imidazo[1,2-a]pyrazine-6-carboxylic acid and 8-hydroxy-2-(tetrahydro-2H-pyran-4-yl)imidazo[1,2-a]pyrazine-6-carboxylic acid (Preparation 74A and 74B, 68.4 mg, 0.247 mmol), 6-(difluoromethyl)pyridin-2-amine (53 mg, 0.370 mmol) in pyridine (2 mL) in a 2-dram vial, was added T3P® (50 wt. % in EtOAc)® (785 mg, 1.23 mmol, 50% purity) at rt. The vial was capped and stirred in a heating block at 80° C. overnight. The cooled mixture was partitioned between EtOAc and H₂O and the organic phase washed with brine, dried (MgSO₄), evaporated to dryness in vacuo and the residue purified by prep HPLC (SunFire C18 column, 60 mL/min flow rate, MeCN/H₂O/0.1% TFA; Gradient (% organic): 10-70) to afford the title compound (Example 206, N-[6-(difluoromethyl)-2-pyridyl]-8-methoxy-2-tetrahydropyran-4-ylimidazo[1,2-a]pyrazine-6-carboxamide trifluoroacetate) as a white solid (15 mg, 15% yield) and N-(6-(difluoromethyl)pyridin-2-yl)-8-hydroxy-2-(tetrahydro-2H-pyran-4-yl)imidazo[1,2-a]pyrazine-6-carboxamide which was used in Part B below. LCMS m/z=404 [M+H]⁺; NMR (500 MHz, CDCl₃) δ: 1.83-1.97 (m, 2H), 2.12 (br dd, 2H), 3.29 (tt, 1H), 3.65 (td, 2H), 4.09-4.22 (m, 2H), 4.30-4.43 (m, 3H), 6.48-6.76 (m, 1H), 7.48-7.53 (m, 2H), 7.61-7.66 (m, 1H), 7.92-8.03 (m, 1H), 8.53 (d, 1H), 8.73-8.84 (m, 1H), 9.96 (s, 1H).

Part B. A mixture of N-(6-(difluoromethyl)pyridin-2-yl)-8-hydroxy-2-(tetrahydro-2H-pyran-4-yl)imidazo[1,2-a]pyrazine-6-carboxamide (23.8 mg, 0.061 mmol), K₂CO₃ (42.24 mg, 0.305 mmol) and EtI (9.53 mg, 0.061 mmol) in DMF (2 mL) in a vial was capped and heated at 100° C. overnight. The mixture was filtered through a pad of Celite® and the filtrate evaporated to dryness in vacuo. The residue was purified by mass directed prep-HPLC (SunFire C18 column, 60 mL/min flow rate, MeCN/H₂O/0.1% TFA; Gradient (% organic) 10-70) to afford N46-(difluoromethyl)-2-pyridyl]-8-ethoxy-2-tetrahydropyran-4-ylimidazo[1,2-a]pyrazine-6-carboxamide trifluoroacetate (Example 207, 3.4 mg, 10.5% yield). LCMS m/z=418 [M+H]⁺; ¹H NMR (400 MHz, CDCl₃) δ: 1.59-1.70 (m, 3H), 1.86 (br d, 2H), 2.07-2.17 (m, 1H), 3.26 (br d, 1H), 3.57-3.68 (m, 2H), 4.13 (br d, 2H), 4.74-4.87 (m, 2H), 6.44-6.79 (m, 1H), 7.48 (d, 1H), 7.58 (s, 1H), 7.96 (t, 1H), 8.50-8.59 (m, 1H), 8.69-8.77 (m, 1H).

### Examples 208-211

The title compounds were prepared in an analogous manner to that described for Examples 206 and 207 using the appropriate amine as shown in the following table:

| Example | Name/Structure | Amine/Yield/Data |
|---|---|---|
| 208 (Part A) | 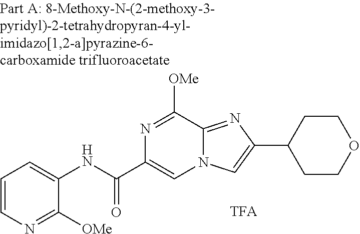 [SMILES: COc1ncccc1NC(=O)c1cn2cc(C3CCOCC3)nc2c(OC)n1] | Amine: 2-methoxypyridin-3-amine Part A. White solid (16.2 mg, 16%). LCMS m/z = 384 [M + H]⁺ ¹H NMR (500 MHz, CDCl₃) δ: 1.81- 1.97 (m, 2H), 2.12 (br dd, 2H), 3.28 (tt, 1H), 3.65 (td, 2H), 4.10-4.21 (m, 5H), 4.28-4.40 (m, 3H), 7.07 (dd, 1H), 7.59-7.69 (m, 1H), 8.01 (dd, 1H), 8.68-8.78 (m, 1H), 8.82 (dd, 1H), 9.59 (br s, 3H), 10.09 (s, 1H). |
| 209 (Part B) | 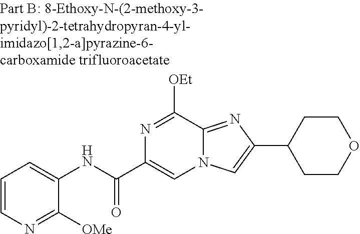 [SMILES: CCOc1nc(C(=O)Nc2cccnc2OC)cn2cc(C3CCOCC3)nc12] | Part B. Yield: 1.2 mg, 1.5% LCMS m/z = 398 [M + H]⁺ |
| 210 211 (Part B) | 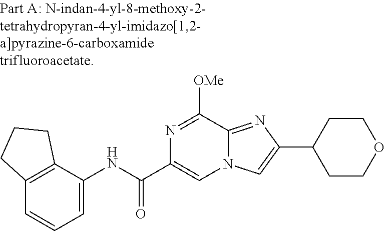 [SMILES: COc1nc(C(=O)Nc2cccc3c2CCC3)cn2cc(C3CCOCC3)nc12] | Amine: 2,3-dihydro-lH-inden-4- amine. Part A. White solid (9.9 mg, 8.7%). LCMS m/z = 393 [M + H]⁺ 1H NMR (500 MHz, CDCl₃) δ: 1.88 (qd, 2H), 2.10 (br dd, 2H), 2.17-2.28 (m, 2H), 2.94- 3.08 (m, 4H), 3.23 (tt, 1H), 3.62 (td, 2H), 4.14 (dd, 2H), 4.26-4.35 (m, 3H), 7.12 (d, 1H), 7.24-7.28 (m, 1H), 7.46-7.57 (m, 1H), 7.59-7.72 (m, 2H), 8.11 (d, 1H), 8.75-8.83 (m, 1H), 9.51 (s, 1H). |
| | 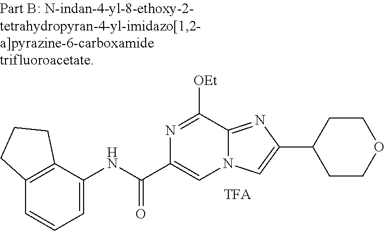 [SMILES: CCOc1nc(C(=O)Nc2cccc3c2CCC3)cn2cc(C3CCOCC3)nc12] | Part B Yield: 2.9 mg, 4% LCMS m/z = 407 [M + H]⁺ 1H NMR (400 MHz, CDCh) δ: 1.62 (t, 3H), 1.81-1.95 (m, 2H), 4.72 (q, 2H), 7.12 (d, 1 H), (m, 2H), 2.91-3.09 (m, 4H), 3.21- 3.33 (m, 1H), 3.63 (td, 2H), 4.14 (dd, 2H), 4.72 (q, 2H), 7.12 (d, 1 H), 7.23-7.28 (m, 1H), 7.59 (d, 1H), 8.12 (d, 1 H) 8.76 (s, 1 H) 9.46 (s, 1H). |

### Example 212: N-(5-fluoro-1-methyl-2-oxo-1,2-dihydropyridin-3-yl)-7-methoxy-2-(tetrahydro-2H-pyran-4-yl)imidazo[1,2-a]pyridine-6-carboxamide trifluoroacetate

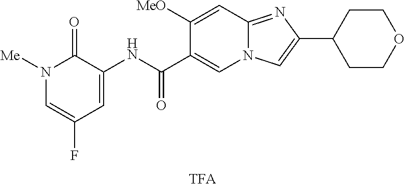 [SMILES: COc1cc2nc(C3CCOCC3)cn2cc1C(=O)Nc1cc(F)cn(C)c1=O]

To a mixture of 7-methoxy-2-(tetrahydro-2H-pyran-4-yl)imidazo[1,2-a]pyridine-6-carboxylic acid (Preparation 75, 150 mg, 0.543 mmol), 3-amino-5-fluoro-1-methylpyridin-2(1H)-one (Preparation 139, 84.9 mg, 0.597 mmol) in Pyridine (2 mL) in a 2-dram vile was added T3P® (50 wt. % in EtOAc)® (1.73 g, 2.71 mmol, 50% purity) at rt. The vial was capped and stirred at 22° C. overnight. The mixture was diluted with EtOAc and H₂O and organic phase washed with brine, dried (MgSO₄), and evaporated to dryness in vacuo. The residue was purified by mass-directed prep HPLC (Sunfire Prep C18 5 μm 30×50 mm; 10%-70% MeCN/H₂O+0.1% TFA) to afford N-(5-fluoro-1-methyl-2-oxo-1,2-dihydropyridin-3-yl)-7-methoxy-2-(tetrahydro-2H-pyran-4-yl)imidazo[1,2-a]pyridine-6-carboxamide trifluoroacetate as a white solid (9.1 mg, 4.2%). LCMS m/z 401 [M+H]⁺; ¹H NMR (400 MHz, MeOH-d₄) δ: 1.69-1.81 (m, 2H), 1.94 (br d, 2H), 3.01-3.09 (m, 1H), 3.46-3.54 (m, 2H), 3.56 (s, 3H), 3.97 (br dd, 2H), 4.21 (s, 3H), 7.21 (s, 1H), 7.41 (dd, 1H), 7.81 (s, 1H), 8.46 (d, 1H), 9.25 (s, 1H).

### Examples 213-228

The title compounds were prepared in an analogues method to that described for Example 212 using the appropriate carboxylic acid and appropriate amine as shown in the table below:

| Example | Name/Structure/Reactants/HPLC Conditions | Yield/Data |
|---|---|---|
| 213 | 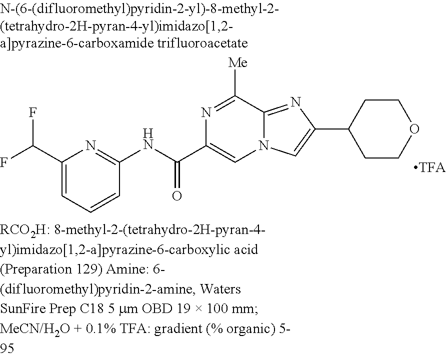 [SMILES: Cc1nc(C(=O)Nc2cccc(C(F)F)n2)cn2cc(C3CCOCC3)nc12] | 3.4 mg, 2.5% LCMS m/z = 388 (M + H)⁺ ¹H NMR (500 MHz, DMSO-d₆) δ: 1.68-1.80 (m, 2H), 1.93-2.01 (m, 2H), 2.85 (s, 3H), 3.08 (tt, 1H), 3.48-3.53 (m, 2H), 3.96 (dt, 2H), 6.85-7.19 (m, 1H), 7.52 (d, 1H), 8.10-8.16 (m, 2H), 8.45 (d, 1H), 9.29 (s, 1H), 10.38 (s, 1H). |
| 214 | 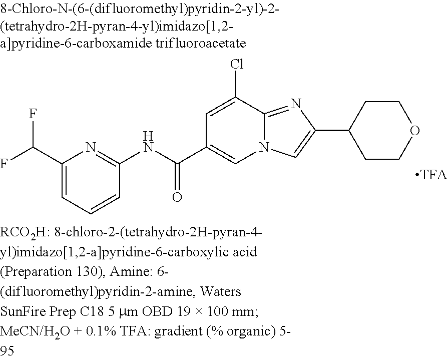 [SMILES: O=C(Nc1cccc(C(F)F)n1)c1cc(Cl)c2nc(C3CCOCC3)cn2c1] | 88.7 mg, 68% LCMS m/z = 407 (M + H)⁺ ¹H NMR (500 MHz, DMSO-d₆) δ: 1.67-1.80 (m, 2H), 1.96 (brdd, 2H), 3.03 (tt, 1H), 3.44-3.51 (m, 2H), 3.95 (br dd, 2H), 6.83-7.07 (m, 1H), 7.49 (d, 1H), 8.00 (s, 2H), 8.07 (t, 1H), 8.33 (d, 1H), 9.32 (d, 1H), 11.26 (s, 1H). |
| 215 | 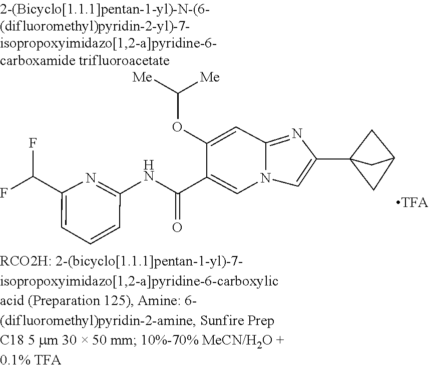 [SMILES: CC(C)Oc1cc2nc(C34CC(C3)C4)cn2cc1C(=O)Nc1cccc(C(F)F)n1] | white solid; 64.5 mg, 40% LCMS m/z = 413 [M + H]⁺ ¹H NMR (400 MHz, MeOH-d₄) δ: 1.58 (d, 6H), 2.31 (s, 6H), 2.66 (s, 1H), 4.92 (quintet, 1H), 6.60 (t, 1H), 7.30 (s, 1H), 7.45 (d, 1H), 7.84 (s, 1H), 8.00 (t, 1H), 8.39-8.41 (m, 1H), 9.19 (s, 1H). |
| 216 | 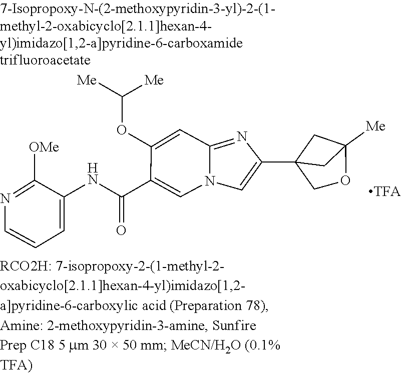 [SMILES: COc1ncccc1NC(=O)c1cn2cc(C34COC(C)(C3)C4)nc2cc1OC(C)C] | White solid (23 mg, 27%) LCMS m/z = 423 [M + H]⁺ ¹H NMR (500 MHz, MeOH-d₄) δ: 1.52 (s, 3H), 1.64 (d, 6H), 1.97-2.05 (m, 2H), 2.21 (dd, 2H), 4.01-4.12, (m, 5H), 5.20 (d, 1H), 7.03 (d, 1H), 7.32 (s, 1H), 7.93 (dd, 1H), 8.03 (s, 1H), 8.79 (d, 1H), 9.37 (s, 1H). |
| 217 | 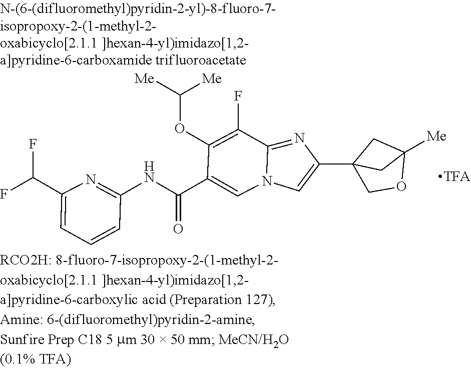 [SMILES: CC(C)Oc1c(C(=O)Nc2cccc(C(F)F)n2)cn2cc(C34COC(C)(C3)C4)nc2c1F] | White solid (15.7 mg, 9.2%) LCMS m/z = 461 [M + H]⁺ ¹H NMR (500 MHz, MeOH-d₄) δ: 1.43-1.65 (m, 10H), 1.99 (dd, 2H), 2.22 (dd, 2H), 4.05 (s, 2 H), 5.13 (br s, 1H), 6.45- 6.82 (m, 1H), 7.48 (d, 1H), 7.91-8.12 (m, 2H), 8.33-8.47 (m, 1H), 9.05 (s, 1 H). |
| 218 | 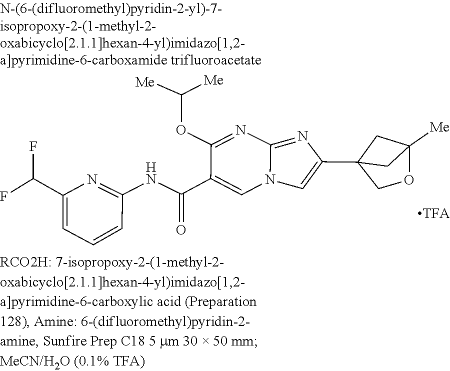 [SMILES: CC(C)Oc1nc2nc(C34COC(C)(C3)C4)cn2cc1C(=O)Nc1cccc(C(F)F)n1] | 31.6 mg, 20% LCMS m/z = 444 [M + H]⁺ ¹H NMR (500 MHz, MeOH-d₄) δ: 1.52 (s, 3H), 1.60 (brd, 6H), 1.98 (dd, 2H), 2.14-2.24 (m, 2H), 4.04 (s, 2H), 5.68 (br s, 1H), 6.63 (s, 1H), 7.49 (d, 1H), 7.90 (s, 1H), 8.03 (t, 1H), 8.37-8.49 (m, 1H), 9.49 (br s, 1 H). |
| 219 | 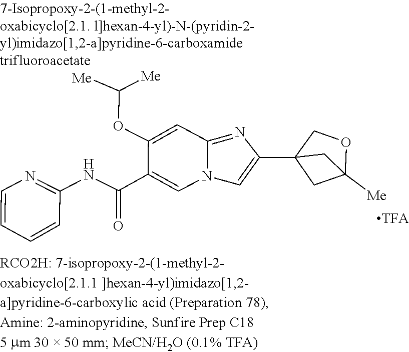 [SMILES: CC(C)Oc1cc2nc(C34COC(C)(C3)C4)cn2cc1C(=O)Nc1ccccn1] | 26.2 mg, 32.8% ¹H NMR (500 MHz, MeOH-d₄) δ: 1.44-1.71 (m, 10H), 1.99 (dd, 2H), 2.24 (dd, 2H), 4.05 (s, 2 H), 5.09 (dt, 1H), 7.29 (dd, 1H), 7.36 (s, 1H), 7.87-8.03 (m, 2H), 8.19 (br d, 1H), 8.39 (br d, 1H), 9.22 (s, 1H). |
| 220 | 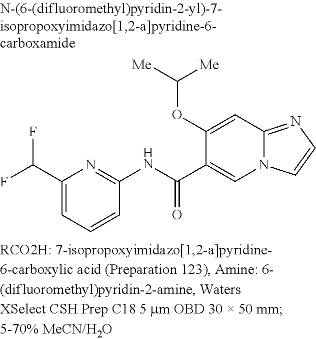 [SMILES: CC(C)Oc1cc2nccn2cc1C(=O)Nc1cccc(C(F)F)n1] | Solid; 10 mg, 7% LCMS m/z = 347 [M + H]⁺ ¹H NMR (500 MHz, DMSO-d₆) δ: 1.42 (brs, 6H), 4.94-5.05 (m, 1H), 6.75-7.06 (m, 1H), 7.38 (s, 1H), 7.51 (brd, 1H), 7.99 (d, 1H), 8.10 (t, 1H), 8.16 (d, 1H), 8.29-8.42 (m, 1H), 9.24 (s, 1H). |
| 221 | 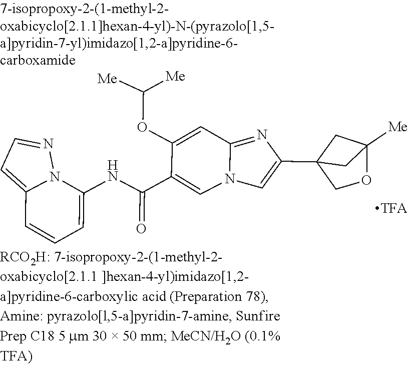 [SMILES: CC(C)Oc1cc2nc(C34COC(C)(C3)C4)cn2cc1C(=O)Nc1cccc2ccnn12] | Yellow solid (9.6 mg, 11%) ¹H NMR (500 MHz, DMSO-d₆) δ: 1.46 (s, 5H), 1.63 (d, 8H), 1.85 (brd, 2H), 2.10 (br s, 3H), 3.94 (s, 2H), 5.26 (br s, 1H) 6.76 (d, 1H), 6.88-7.23 (m, 1H), 7.27-7.45 (m, 2H), 7.55 (d, 1H), 7.88 (d, 1H), 8.16 (d, 1H). |
| 222 | 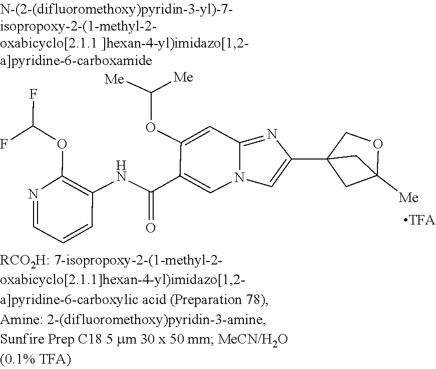 [SMILES: CC(C)Oc1cc2nc(C34COC(C)(C3)C4)cn2cc1C(=O)Nc1cccnc1OC(F)F] | White solid (17.7 mg, 20%) LCMS m/z = 459 [M + H]⁺ ¹H NMR (500 MHz, DMSO-d₆) δ: 1.38-1.55 (m, 9H), 1.85 (brd, 2H), 2.11 (br d, 2H), 3.93 (s, 2H), 5.06-5.21 (m, 1H), 6.87- 7.27 (m, 1H), 7.27- 7.48 (m, 2H), 8.02 (s, 1H), 7.65-7.95 (m, 1H), 8.06 (dd, 1H), 8.76 (brd, 1H), 9.33 (s, 1H), 10.05 (s, 1H). |
| 223 | 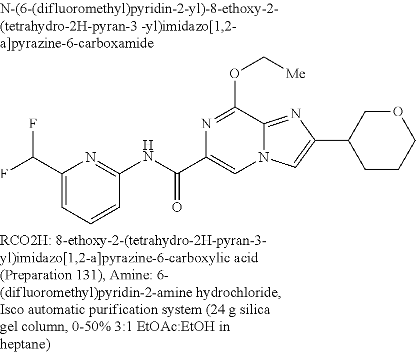 [SMILES: CCOc1nc(C(=O)Nc2cccc(C(F)F)n2)cn2cc(C3CCCOC3)nc12] | White Solid; 109 mg LCMS m/z 418 [M + H]⁺ ¹H NMR (400 MHz, MeOH-d₄) δ: 1.60 (t, 3H), 1.75-1.78 (m, 2H), 1.88- 1.91 (m, 1H), 2.17-2.22 (m, 1H), 3.07-3.13 (m, 1H), 3.53-3.63 (m, 2H), 3.90-3.95 (m, 1H), 4.11- 4.15 (m, 1H), 4.79 (q, 2H), 6.69 (t, 1H), 7.45 (d, 1H), 7.95 (s, 1H), 8.02 (t, 1H), 8.51 (d, 1H), 8.88 (s, 1H). |
| 224 | 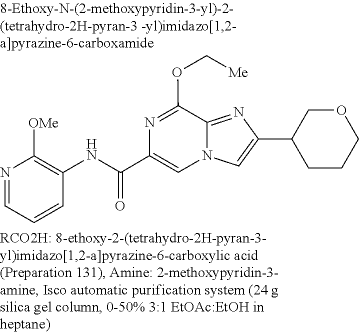 [SMILES: CCOc1nc(C(=O)Nc2cccnc2OC)cn2cc(C3CCCOC3)nc12] | White solid, 39 mg (19%) LCMS m/z 398 [M + H]⁺. ¹H NMR (400 MHz, CDCl₃) δ: 1.66 (t, 3H), 1.72-1.77 (m, 2H), 1.92- 2.00 (m, 1H), 2.17-2.25 (m, 1H), 3.19-3.25 (m, 1H), 3.57-3.63 (m, 1H), 3.66-3.71 (m, 1H), 3.90- 3.96 (m, 1H), 4.10 (s, 3H), 4.17 (dd, 1H), 4.80 (q, 2H), 6.97-7.01 (m, 1H), 7.61 (s, 1H), 7.93 (dd, 1H), 8.63 (s, 1H), 8.77 (d, 1H), 10.15 (s, 1H). |
| 225 | 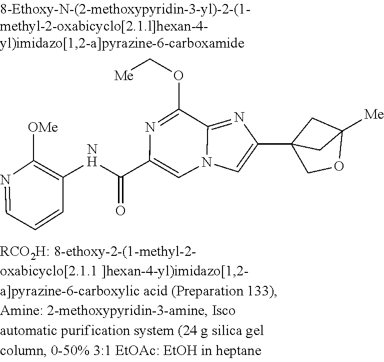 [SMILES: CCOc1nc(C(=O)Nc2cccnc2OC)cn2cc(C34COC(C)(C3)C4)nc12] | White solid; 38 mg (52%) LCMS m/z = 410 [M + H]⁺. ¹H NMR (400 MHz, MeOH-d₄) δ: 1.53 (s, 3H), 1.66 (t, 3H), 8.84 (s, 1H), 8.70 (d, 1H), 8.02 (s, 1H), 1.93-1.96 (m, 2H), 2.17-2.21 (m, 2H), 4.07 (s, 2H), 4.11 (s, 3H), 4.80 (q, 2H), 7.02 (dd, 1H), 7.91 (dd, 1H). |
| 226 | 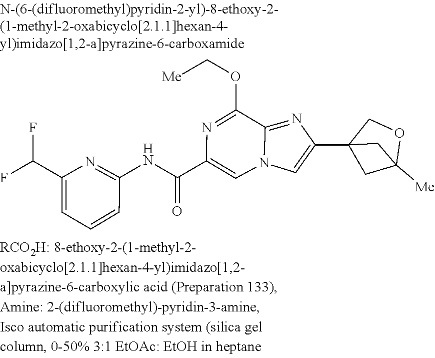 [SMILES: CCOc1nc(C(=O)Nc2cccc(C(F)F)n2)cn2cc(C34COC(C)(C3)C4)nc12] | Off-white solid: 46 mg, 60% LCMS m/z = 430 [M + H]⁺. ¹H NMR (400 MHz, MeOH-d₄) δ: 1.53 (s, 3H), 1.63 (t, 3H), 1.94- 1.97 (m, 2H), 2.16-2.20 (m, 2H), 4.07 (s, 2H), 4.83 (q, 2H), 6.72 (t, 1H), 7.49 (d, 1H), 8.02 (s, 1H), 8.05 (t, 1H), 8.54 (d, 1H), 8.92 (s, 1H). |
| 227 | 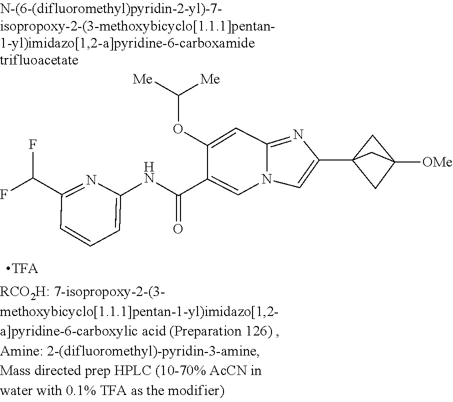 [SMILES: COC12CC(c3cn4cc(C(=O)Nc5cccc(C(F)F)n5)c(OC(C)C)cc4n3)(C1)C2] | White solid: 18 mg, 13% LCMS m/z = 443 [M + H]⁺. ¹H NMR (400 MHz, MeOH-d₄) δ: 1.59 (d, 6H), 2.39 (s, 6H), 3.40 (s, 3H), 5.11 (quintet, 1H), 6.64 (t, 1H), 7.30 (s, 1H), 7.49 (d, 1H), 7.93 (s, 1H), 8.04 (t, 1H), 8.42-8.45 (brm, 1H), 9.21 (s, 1H). |
| 228 | 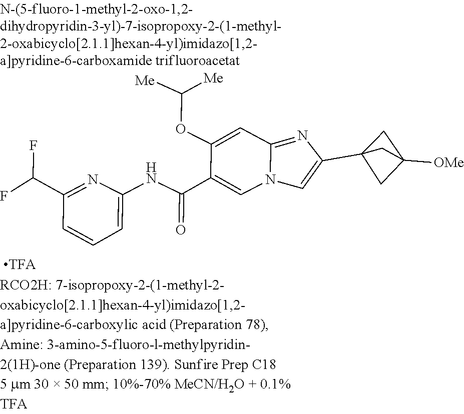 [SMILES: COC12CC(c3cn4cc(C(=O)Nc5cccc(C(F)F)n5)c(OC(C)C)cc4n3)(C1)C2] | Yellow oil: 54 mg, 27% LCMS m/z = 441 [M + H]⁺. ¹H NMR (400 MHz, MeOH-d₄) δ: 1.55 (s, H), 1.68 (d, 6H), 1.99- 2.03 (m, 2H), 2.22-2.27 (m, 2H), 3.66 (s, 3H), 4.07 (s, 2H), 5.20 (quintet, 1H), 7.41 (s, 1H), 7.50 (dd, 1H), 8.04 (s, 1H), 8.58 (dd, 1H), 9.40 (s, 1H). |

The following codes refer to the preparative HPLC conditions used as indicated in the example procedures. Individual gradients were optimised for each example as appropriate.

| Prep-HPLC | |
|---|---|
| Code | Conditions |
| prep-HPLC-A | Phenomenex Synergi, C18 150 × 30 mm, 4 μm; |
| | MeCN/H₂O + 0.05% HCl; gradient 0-100% |
| prep-HPLC-B | Phenomenex Synergi C18 150 × 30 mm, 5 μm; |
| | MeCN/H₂O + 0.1% HCl; gradient 0-100% |
| prep-HPLC-C | YMC Actus Triart C18; 150 × 30 5 μm, |
| | MeCN/H₂O + 0.225% HCO₂H; gradient 0-100% |
| prep-HPLC-D | Waters SunFire C18 100 × 100 mm, 5 μm: |
| | MeCN/H₂O + 0.1% TFA; gradient 0-100% |
| prep-HPLC-E | Waters SunFire C18 100 × 19 mm, 5 μm; |
| | MeOH/H₂O + NH₄OH; gradient 0-100% |
| prep-HPLC-F | Waters XSelect CSH Prep C18 100 × 19, 5 μm; |
| | MeCN/H₂O + 0.1% NH₄OH; gradient 0-100% |
| prep-HPLC-G | YMC Actus Triart C18 100 × 20, 5 μm; |
| | MeOH/H₂O + 0.01% NH₄OH; gradient 0-100% |
| prep-HPLC-H | Angela DuraShell C18; 150 × 25, 5 μm, |
| | MeCN/H₂O + 0.04% NH₄OH + |
| | 10 mM NH₄HCO₃; gradient 0-100% |
| prep-HPLC-I | Phenomenex Synergi C18 150 × 30, 4 μm, |
| | MeCN/H₂O + 0.05% NH₄HCO₃; gradient 0-100% |
| prep-HPLC-J | Welch Xtimate C18 150 × 30 mm, 5 μm; |
| | MeCN/H₂O + 10 mM NH₄HCO₃; gradient 0-100% |
| prep-HPLC-K | Welch Xtimate C18 150 × 25 mm, 5 μm; |
| | MeCN/H₂O + 10 mM NH₄HCO₃; gradient 0-100% |
| prep-HPLC-L | Waters SunFire C18 100 × 19 mm, 5 μm; |
| | MeCN/H₂O; gradient 0-100% |

### Example 229: 3-Chloro-N-(6-(difluoromethyl)pyridin-2-yl)-7-isopropoxy-2-(1-methyl-2-oxabicyclo[2.1.1]hexan-4-yl)imidazo[1,2-a]pyridine-6-carboxamide

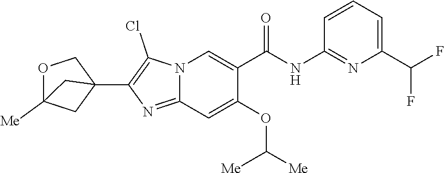 [SMILES: CC(C)Oc1cc2nc(C34COC(C)(C3)C4)c(Cl)n2cc1C(=O)Nc1cccc(C(F)F)n1]

1-Chloropyrrolidine-2,5-dione (12.1 mg, 0.090 mmol) was added to a solution of N-(6-(difluoromethyl)pyridin-2-yl)-7-isopropoxy-2-(1-methyl-2-oxabicyclo[2.1.1]hexan-4-yl)imidazo[1,2-a]pyridine-6-carboxamide (Example 96, 40 mg, 0.090 mmol) in THF (1 mL) and EtOH (1 mL) at 0° C. and the reaction stirred at rt for 1.5 h. The reaction was quenched with aq. sat. NaHCO₃, extracted with EtOAc (3×) and the combined organics washed with brine, dried (MgSO₄), and evaporated to dryness in vacuo. The residue was purified by prep-HPLC to afford 3-chloro-N-(6-(difluoromethyl)pyridin-2-yl)-7-isopropoxy-2-(1-methyl-2-oxabicyclo[2.1.1]hexan-4-yl)imidazo[1,2-a]pyridine-6-carboxamide (15 mg, 34.8%). LCMS m/z=477.1 [M+H]⁺; ¹H NMR (500 MHz, DMSO-d₆) δ: 1.41 (br d, 6H), 1.45 (s, 3H), 1.82-1.91 (m, 2H), 2.16 (dd, 2H), 3.97 (s, 2H), 4.93 (spt, 1H), 6.79-7.01 (m, 1H), 7.27 (s, 1H), 7.49 (d, 1H), 8.09 (t, 1H), 8.37 (br d, 1H), 8.67 (s, 1H), 10.97 (s, 1H).

### Example 230: 8-Fluoro-7-isopropoxy-2-(1-methyl-2-oxabicyclo[2.1.1]hexan-4-yl)-N-(6-(trifluoromethyl)pyridin-2-yl)imidazo[1,2-a]pyridine-6-carboxamide trifluoroacetate

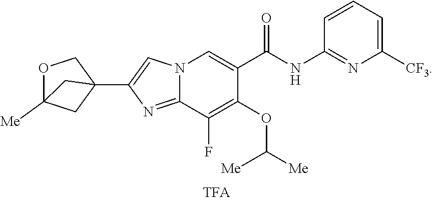 [SMILES: CC(C)Oc1c(C(=O)Nc2cccc(C(F)(F)F)n2)cn2cc(C34COC(C)(C3)C4)nc2c1F]

T3P® (50 wt. % in EtOAc, 171 mg, 0.269 mmol) and TEA (45.4 mg, 0.449 mmol) were added to a solution of 8-fluoro-7-isopropoxy-2-(1-methyl-2-oxabicyclo[2.1.1]hexan-4-yl)imidazo[1,2-a]pyridine-6-carboxylic acid (Preparation 127, 30 mg, 0.090 mmol) and 6-(trifluoromethyl)pyridin-2-amine (18.9 mg, 0.117 mmol) in DMF (1 mL) and the reaction stirred at 50° C. overnight. The cooled mixture was purified by prep-HPLC-B to afford 8-fluoro-7-isopropoxy-2-(1-methyl-2-oxabicyclo[2.1.1]hexan-4-yl)-N-(6-(trifluoromethyl)pyridin-2-yl)imidazo[1,2-a]pyridine-6-carboxamide trifluoroacetate. LCMS m/z=479.0 [M+H]⁺; ¹H NMR (500 MHz, MeOH-d₄) δ: 1.48-1.57 (m, 10H), 1.93 (dd, 2H), 2.18 (dd, 2H), 4.06 (s, 2H), 7.60 (d, 1H), 7.91 (d, 1H), 8.05-8.15 (m, 1H), 8.56 (br d, 1H), 9.01 (s, 1H).

### Example 231: N-(6-(difluoromethyl)pyridin-2-yl)-8-methoxy-2-(1-methyl-2-oxabicyclo[2.1.1]hexan-4-yl)imidazo[1,2-a]pyrazine-6-carboxamide

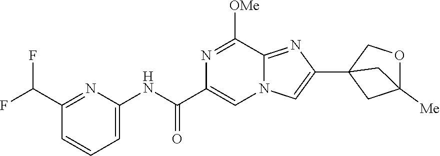 [SMILES: COc1nc(C(=O)Nc2cccc(C(F)F)n2)cn2cc(C34COC(C)(C3)C4)nc12]

To 8-methoxy-2-(1-methyl-2-oxabicyclo[2.1.1]hexan-4-yl)imidazo[1,2-a]pyrazine-6-carboxylic acid (Preparation 132, 27.5 mg, 0.082 mmol) and 6-(difluoromethyl)pyridin-2-amine (17.7 mg, 0.098 mmol, HCl) was added TEA (0.4 mL) and T3P® (50 wt. % in EtOAc, 567 mg, 0.885 mmol, 0.4 mL). The mixture was heated under microwave conditions at 100° C. for 45 min. The reaction was quenched with MeOH and the mixture partitioned between H₂O and EtOAc. The aqueous layer was re-extracted (×2) and the combined organics were evaporated to dryness and the residue purified by column chromatography (SiO₂, 50-100% EtOAc/heptane) to afford N-(6-(difluoromethyl)pyridin-2-yl)-8-methoxy-2-(1-methyl-2-oxabicyclo[2.1.1]hexan-4-yl)imidazo[1,2-a]pyrazine-6-carboxamide as a white powder (24 mg, 70%). LCMS m/z=416.2 [M+H]⁺; ¹H NMR (400 MHz, MeOH-d₄) δ: 1.41 (s, 3H), 1.72-1.85 (m, 2H), 2.00-2.13 (m, 2H), 3.95 (s, 2H), 4.24 (s, 3H), 6.40-6.78 (m, 1H), 7.36 (d, 1H), 7.86-8.01 (m, 2H), 8.41 (d, 1H), 8.81 (s, 1H).

### Example 232-251

The title compounds were prepared from the appropriate carboxylic acid and amine using an analogous method to that described for Example 231 as shown in the following table.

| Ex | Name/Structure/RCO₂H | Data |
|---|---|---|
| 232 | 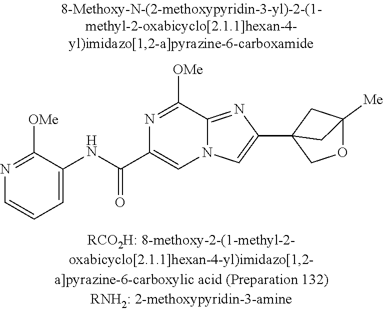 [SMILES: COc1ncccc1NC(=O)c1cn2cc(C34COC(C)(C3)C4)nc2c(OC)n1] | 12 mg, 50% LCMS m/z = 396.2 [M + H]⁺ ¹H NMR (400 MHz, MeOH- d₄) δ: 1.53 (s, 3H), 1.87-1.96 (m, 2H), 2.12-2.22 (m, 2H), 4.06 (s, 2H), 4.10 (s, 3H), 4.32 (s, 3H), 7.01 (dd, 1H), 7.90 (dd, 1H), 8.02 (s, 1H), 8.70 (dd, 1H), 8.85 (s, 1H). |
| 233 | 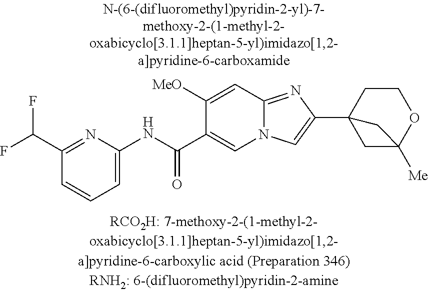 [SMILES: COc1cc2nc(C34CCOC(C)(C3)C4)cn2cc1C(=O)Nc1cccc(C(F)F)n1] | 4.1 mg, 38% LCMS m/z = 429.3 [M + H]⁺ ¹H NMR (400 MHz, MeOH- d₄) δ: 1.25-1.35 (s, 4H), 2.21- 2.29 (m, 4H), 2.38 (t, 2H), 4.05 (s, 3H), 4.11 (t, 2H), 6.50 (t, 1H), 6.89 (s, 1H), 7.35 (d, 1H), 7.49 (s, 1H), 7.90 (t, 1H), 8.36 (d, 1H), 8.95 (s, 1H). |
| 234 | 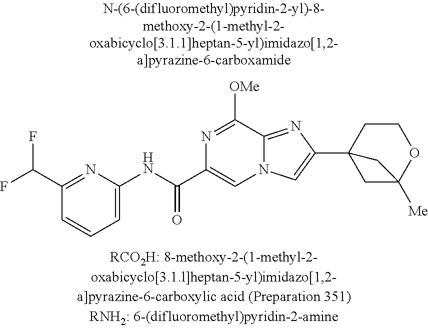 [SMILES: COc1nc(C(=O)Nc2cccc(C(F)F)n2)cn2cc(C34CCOC(C)(C3)C4)nc12] | 33 mg, 80% LCMS m/z = 430.3 [M + H]⁺ ¹H NMR (400 MHz, MeOH- d₄) δ: 1.33 (s, 3H), 2.25-2.34 (m, 4H), 2.42 (t, 2H), 4.24 (t, 2H), 4.36 (s, 3H), 6.54-6.88 (m, 1H), 7.49 (d, 1H), 7.93 (s, 1H), 8.06 (t, 1H), 8.55 (dd, 1H), 8.93 (s, 1H). |
| 235 | 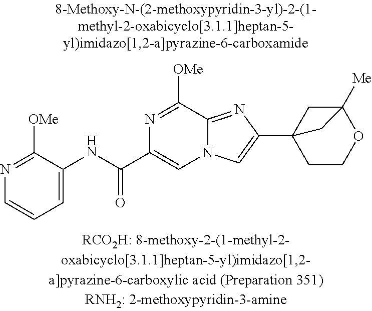 [SMILES: COc1ncccc1NC(=O)c1cn2cc(C34CCOC(C)(C3)C4)nc2c(OC)n1] | 20 mg, 65% LCMS m/z = 410.3 [M + H]⁺ ¹H NMR (400 MHz, MeOH-d₄) δ:: 1.33 (s, 3H), 2.23-2.35 (m, 4H), 2.42 (t, 2H), 4.11 (s, 3H), 4.17-4.28 (m, 2H), 4.33 (s, 3H), 7.03 (dd, 1H), 7.87-7.95 (m, 2H), 8.71 (dd, 1H), 8.85 (s, 1H). |
| 236 | 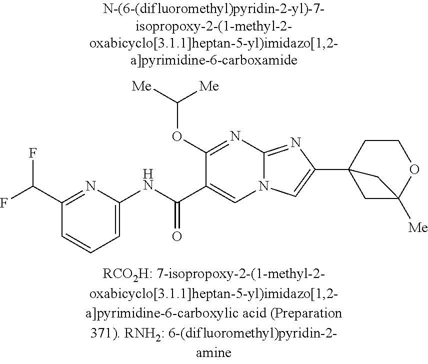 [SMILES: CC(C)Oc1nc2nc(C34CCOC(C)(C3)C4)cn2cc1C(=O)Nc1cccc(C(F)F)n1] | 26.8 mg, 63% ¹H NMR (400 MHz, MeOH- d₄) δ: 1.33 (s, 3H), 1.62 (d, 6H), 2.16-2.31 (m, 4H), 2.38 (t, 2H), 4.15-4.28 (m, 2H), 5.70 (td, 1H), 6.45-6.81 (m, 1H), 7.46 (s, 1H), 7.52 (s, 1H), 8.02 (t, 1H), 8.45 (d, 1H), 9.38 (s, 1H). |
| 237 | 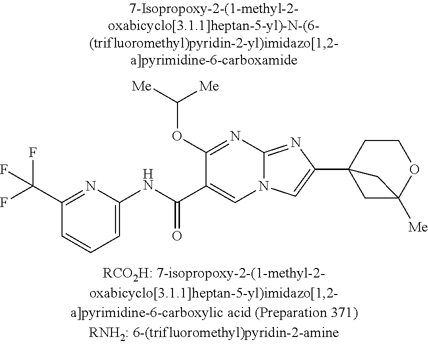 [SMILES: CC(C)Oc1nc2nc(C34CCOC(C)(C3)C4)cn2cc1C(=O)Nc1cccc(C(F)(F)F)n1] | 17 mg, 38% LCMS m/z = 476.3 [M + H]⁺ ¹H NMR (400 MHz, MeOH- d₄) δ: 1.33 (s, 3H), 1.61 (d, 6H), 2.18-2.30 (m, 4H), 2.38 (t, 2H), 4.17-4.27 (m, 2H), 5.69 (td, 1H), 7.52 (s, 1H), 7.58 (d, 1H), 8.08 (t, 1H), 8.56 (d, 1H), 9.39 (s, 1H). |
| 238 | 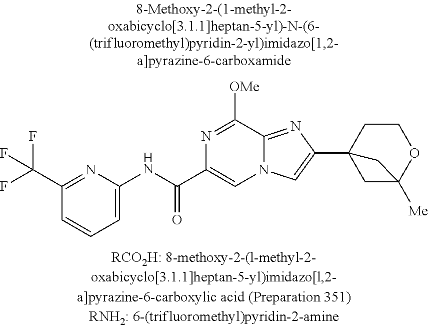 [SMILES: COc1nc(C(=O)Nc2cccc(C(F)(F)F)n2)cn2cc(C34CCOC(C)(C3)C4)nc12] | 23 mg, 53% LCMS m/z = 448.3 [M + H]⁺ ¹H NMR (400 MHz, MeOH- d₄) δ: 8.94 (s, 1H), 8.67 (d, 1H), 8.12 (t, 1H), 7.93 (s, 1H), 7.62 (d, 1H), 4.37 (s, 3H), 4.24 (t, 2H), 2.42 (t, 2H), 2.21-2.35 (m, 4H), 1.33 (s, 3H). |
| 239 | 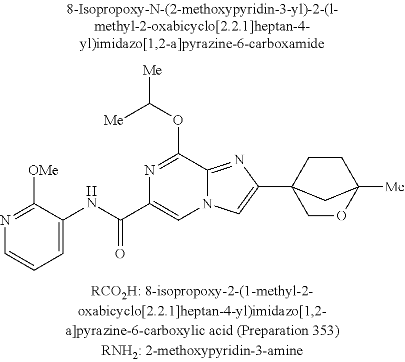 [SMILES: COc1ncccc1NC(=O)c1cn2cc(C34CCC(C)(C3)OC4)nc2c(OC(C)C)n1] | 17 mg, 48% LCMS m/z = 438.3 [M + H]⁺ ¹H NMR (400 MHz, MeOH- d₄) δ: 1.48 (s, 3H), 1.64 (d, 6H), 1.75-2.31 (m, 6H), 3.94- 4.02 (m, 1H), 4.08 (s, 3H), 4.13 (dd, 1H), 5.63 (spt, 1H), 6.97 (dd, 1H), 7.53 (s, 1H), 7.91 (dd, 1H), 8.59 (s, 1H), 8.76 (dd, 1H), 10.15 (s, 1H). |
| 240 | 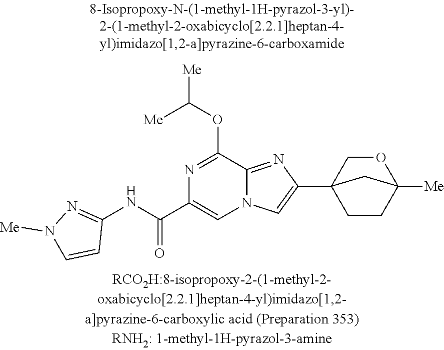 [SMILES: CC(C)Oc1nc(C(=O)Nc2ccn(C)n2)cn2cc(C34CCC(C)(C3)OC4)nc12] | 19.2 mg, 56% LCMS m/z = 411.3 [M + H]⁺ ¹H NMR (400 MHz, MeOH- d₄) δ: 1.48 (s, 3H), 1.55 (d, 6H), 1.76-2.28 (m, 6H), 3.87 (s, 3H), 3.99 (d, 1H), 4.12 (dd, 1H), 5.60-5.80 (m, 1H), 6.84 (d, 1H), 7.32 (d, 1H), 7.51 (s, 1H), 8.61 (s, 1H), 9.66 (s, 1H). |
| 241 | 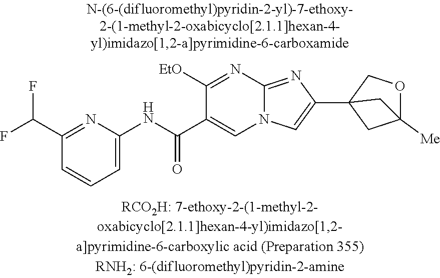 [SMILES: CCOc1nc2nc(C34COC(C)(C3)C4)cn2cc1C(=O)Nc1cccc(C(F)F)n1] | 22 mg, 51% LCMS m/z = 430.2 [M + H]⁺ ¹H NMR (400 MHz, MeOH- d₄) δ: 1.52 (s, 3H), 1.62 (t, 3H), 1.83-1.96 (m, 2H), 2.05- 2.22 (m, 2H), 3.97-4.09 (m, 2H), 4.76 (q, 2H), 6.49-6.84 (m, 1H), 7.47 (d, 1H), 7.58- 7.69 (m, 1H), 8.02 (t, 1H), 8.46 (d, 1H), 9.37 (s, 1H). |
| 242 | 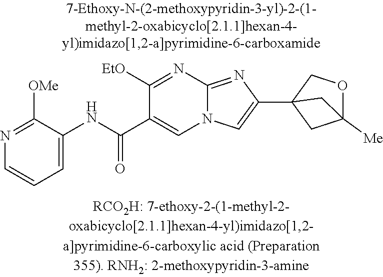 [SMILES: CCOc1nc2nc(C34COC(C)(C3)C4)cn2cc1C(=O)Nc1cccnc1OC] | 26 mg, 63% LCMS m/z = 410.2 [M + H]⁺ ¹H NMR (400 MHz, MeOH- d₄) δ: 1.50 (s, 3H), 1.64 (t, 3H), 1.86 (dd, 2H),2.10(dd, 2H), 4.00 (s, 2H), 4.06 (s, 3H), 4.72 (q, 2H), 6.96 (dd, 1H), 7.60 (s, 1H), 7.86 (dd, 1H), 8.74 (dd, 1H), 9.36 (s, 1H). |
| 243 | 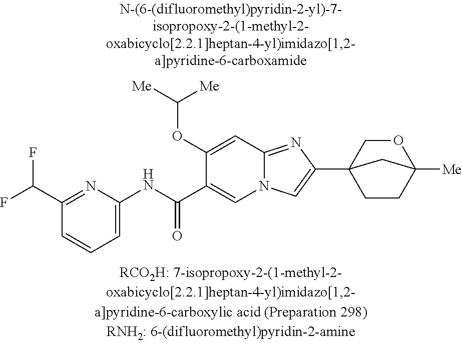 [SMILES: CC(C)Oc1cc2nc(C34CCC(C)(C3)OC4)cn2cc1C(=O)Nc1cccc(C(F)F)n1] | 58.8 mg, 64% LCMS m/z = 457.3 [M + H]⁺ ¹H NMR (400 MHz, MeOH- d₄) δ: 1.48 (s, 3H), 1.60 (d, 6H), 1.74-2.32 (m, 6H), 3.94 (d, 1H), 4.07 (dd, 1H), 4.96- 5.07 (m, 1H), 6.46-6.78 (m, 1H), 7.01 (s, 1H), 7.46 (d, 1H), 7.70 (s, 1H), 8.01 (t, 1H), 8.45 (d, 1H), 9.13 (s, 1H). |
| 244 | 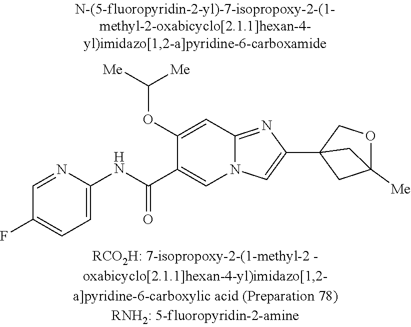 [SMILES: CC(C)Oc1cc2nc(C34COC(C)(C3)C4)cn2cc1C(=O)Nc1ccc(F)cn1] | 21.8 mg, 36% LCMS m/z = 411.2 [M + H]⁺ ¹H NMR (400 MHz, MeOH- d₄) δ: 1.52(s, 3H), 1.59 (d, 6H), 1.79-1.95 (m, 2H), 2.06- 2.20 (m, 2H), 4.03 (s, 2H), 5.00 (td, 1H), 7.01 (s, 1H), 7.60-7.79 (m, 2H), 8.26 (d, 1H), 8.37 (dd, 1H), 9.13 (s, 1H). |
| 245 | 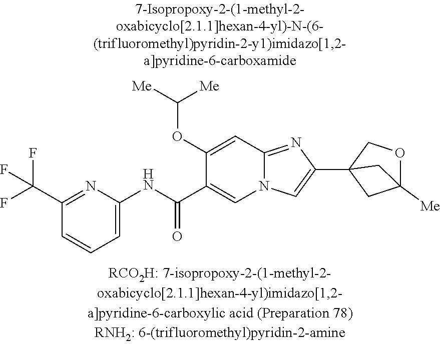 [SMILES: CC(C)Oc1cc2nc(C34COC(C)(C3)C4)cn2cc1C(=O)Nc1cccc(C(F)(F)F)n1] | 22 mg, 61% LCMS m/z = 461.2 [M + H]⁺; ¹H NMR (400 MHz, MeOH- d₄) δ: 1.52 (s, 3H), 1.60 (d, 6H), 1.80-1.98 (m, 2H), 2.03- 2.24 (m, 2H), 4.03 (s, 2H), 4.95-5.10 (m, 1H), 7.02 (s, 1H), 7.57 (d, 1H), 7.74 (s, 1H), 7.94-8.14 (m, 1H), 8.56 (br d, 1H), 9.15 (s, 1H). |
| 246 | 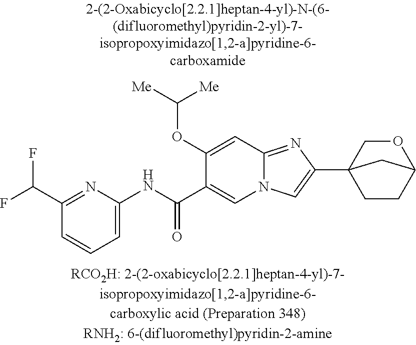 [SMILES: CC(C)Oc1cc2nc(C34CCC(C3)OC4)cn2cc1C(=O)Nc1cccc(C(F)F)n1] | Yield: 60 mg, 67% LCMS m/z = 443.1 [M + H]⁺; ¹H NMR (400 MHz, CDCl₃) δ: 1.61 (d, 6H), 1.74-2.23 (m, 6H), 3.81-3.92 (m, 1H), 4.00 (dd, 1H), 4.72-4.93 (m, 1H), 6.29-6.67 (m, 1H), 7.04-6.95 (m, 1H), 7.32-7.46 (m, 2H), 7.89 (t, 1H), 8.46 (d, 1H), 9.02 (s, 1H), 10.74 (s, 1H). |
| 247 | 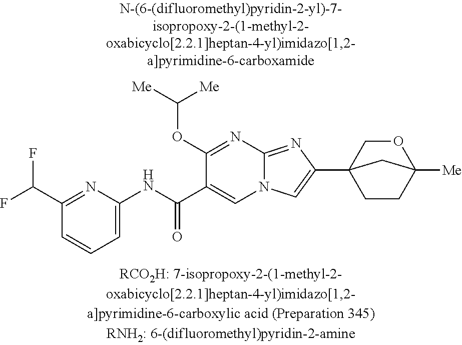 [SMILES: CC(C)Oc1nc2nc(C34CCC(C)(C3)OC4)cn2cc1C(=O)Nc1cccc(C(F)F)n1] | 47 mg, 55% LCMS m/z = 458.2 [M + H]⁺ ¹H NMR (400 MHz, MeOH- d₄) δ: 1.48 (s, 3H), 1.61 (d, 6H), 1.76-2.27 (m, 6H), 3.84- 3.98 (m, 1H), 4.06 (dd, 1H), 5.69 (td, 1H), 6.45-6.80 (m, 1H), 7.47 (d, 1H), 7.61 (s, 1H), 8.02 (t, 1H), 8.45 (br d, 1H), 9.39 (s, 1H). |
| 248 | 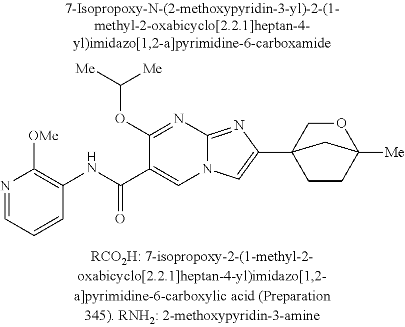 [SMILES: COc1ncccc1NC(=O)c1cn2cc(C34CCC(C)(C3)OC4)nc2nc1OC(C)C] | 62 mg, 70% LCMS m/z = 438.2 [M + H]⁺ ¹H NMR (400 MHz, MeOH- d₄) δ: 1.48 (s, 3H), 1.63 (d, 6H), 1.77-2.28 (m, 6H), 3.93 (d, 1H), 4.06 (dd, 1H), 4.12 (s, 3H), 5.80 (quin, 1H), 7.01 (dd, 1H), 7.61 (s, 1H), 7.91 (dd, 1H), 8.80 (dd, 1H), 9.41 (s, 1H). |
| 249 | 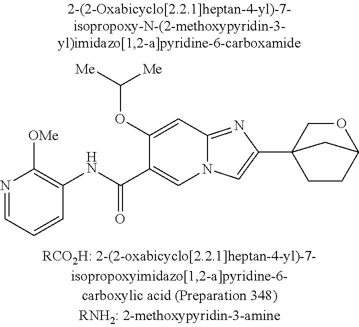 [SMILES: COc1ncccc1NC(=O)c1cn2cc(C34CCC(C3)OC4)nc2cc1OC(C)C] | 58 mg, 68% LCMS m/z = 423.2 [M + H]⁺ ¹H NMR (400 MHz, MeOH- d₄) δ: 1.61 (d, 6H), 1.83-2.27 (m, 6H), 3.81-3.99 (m, 2H), 4.10 (s, 3H), 5.01-5.12 (m, 1H), 6.96-7.09 (m, 2H), 7.73 (d, 1H), 7.91 (dd, 1H), 8.80 (dd, 1H), 9.17 (s, 1H). |
| 250 | 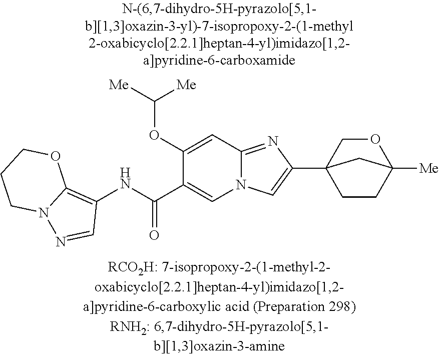 | 23 mg, 96% LCMS m/z = 452.3 [M + H]⁺ ¹H NMR (400 MHz, CDCl₃) δ: 1.50 (s, 3H), 1.61 (d, 6H), 1.85-1.93 (m, 2H), 1.97-2.22 (m, 4H), 2.28-2.39 (m, 2H), 3.89-4.07 (m, 2H), 4.21 (t, 2H), 4.34-4.47 (m, 2H), 5.06 (td, 1H), 7.38 (s, 1H), 7.69 (s, 1H), 7.95 (s, 1H), 9.17 (s, 1H), 9.39 (s, 1H). |
| 251 | 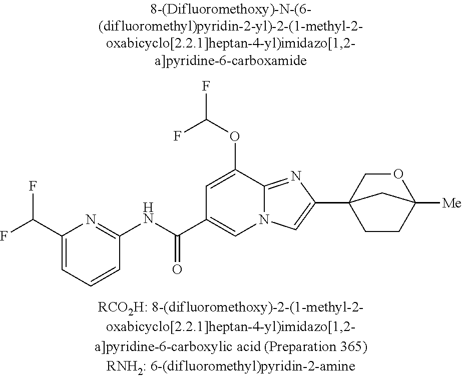 [SMILES: CC12CCC(c3cn4cc(C(=O)Nc5cccc(C(F)F)n5)cc(OC(F)F)c4n3)(CO1)C2] | 93 mg, 100% LCMS m/z = 465.2 [M + H]⁺ ¹H NMR (400 MHz, CDCl₃) δ: 0.95 (s, 1H), 1.30 (m, 2H), 1.50 (s, 3H), 1.80-2.20 (m, 4H), 4.00 (s, 1H), 4.15 (s, 1H), 6.55 (t, 1H), 7.40-7.50 (m, 2H), 7.60 (s, 1H), 7.90 (s, 1H), 8.45-8.55 (m, 2H), 8.75 (s, 1H). |

### Example 252: 7-Isopropoxy-2-(3-methoxybicyclo[1.1.1]pentan-1-yl)-N-(2-methoxypyridin-3-yl)imidazo[1,2-a]pyridine-6-carboxamide trifluoroacetate

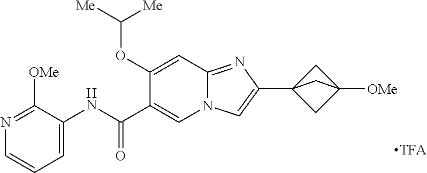 [SMILES: COc1ncccc1NC(=O)c1cn2cc(C34CC(OC)(C3)C4)nc2cc1OC(C)C]

To a mixture of 2-methoxypyridin-3-amine (26.2 mg, 0.211 mmol), 7-isopropoxy-2-(3-methoxybicyclo[1.1.1]pentan-1-yl)imidazo[1,2-a]pyridine-6-carboxylic acid (Preparation 126, 33.4 mg, 0.106 mmol) in Pyridine (2 mL) was added T3P® (50 wt. % in EtOAc, 336 mg, 0.528 mmol) at rt. The vial was capped and stirred at 22° C. for 0.5 h. The mixture was diluted with EtOAc and H₂O and the aqueous phase was extracted with EtOAc (3×15 mL). The combined organics were dried (MgSO₄) and evaporated to dryness in vacuo. The residue was purified by prep-HPLC-D (Gradient: 5-65%) to afford 7-isopropoxy-2-(3-methoxybicyclo[1.1.1]pentan-1-yl)-N-(2-methoxypyridin-3-yl)imidazo[1,2-a]pyridine-6-carboxamide trifluoroacetate as a white solid (33.5 mg, 59%). LCMS m/z=423.4 [M+H]⁺; ¹H NMR (400 MHz, MeOH-d₄) δ: 1.66 (d, 6H), 2.34-2.44 (m, 6H), 3.37-3.44 (m, 3H), 4.11 (s, 3H), 5.15-5.25 (m, 1H), 7.03 (dd, 1H), 7.36 (s, 1H), 7.94 (dd, 1H), 7.96 (s, 1H), 8.78 (dd, 1H), 9.31-9.39 (m, 1H).

### Example 253-413

The title compounds were prepared using the appropriate carboxylic acid and amine building block using an analogous method to that described for Example 252 and using the separation methods shown.

| Ex no | Name/structure/Starting materials | Data |
|---|---|---|
| 253 | 2-(3-Oxabicyclo[3.1.0]hexan-6-yl)-N-(6-(difluoromethyl)pyridin-2-yl)-7- | 15 mg, 21.17% yield as white |
| | isopropoxyimidazo[1,2-a]pyridine-6-carboxamide hydrochloride | solid LCMS m/z = 429.1 |
| | | [M + H]⁺ ¹H NMR (500 MHz, |
| | 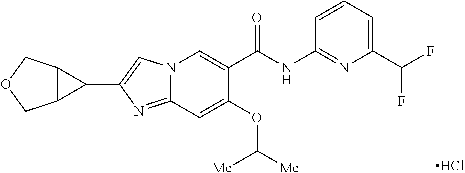 [SMILES: CC(C)Oc1cc2nc(C3C4COCC43)cn2cc1C(=O)Nc1cccc(C(F)F)n1 ; Cl] | DMSO-d₆) δ: 1.41 (d, 6H), 2.01- 2.03 (m, 1H), 2.26 (d, 2H), 3.73 (d, 2H), 3.95 (d, 2H), 5.00 (s, 1H), 6.81-7.03 (m, 1H), 7.26 (s, 1H), 7.51 (d, 1H), 7.94 (s, 1H), 8.10 (t, 1H), 8.35 (s, 1H), 9.16 (s, 1H), 11.11 (s, 1H) |
| | RCO₂H: 2-(3-oxabicyclo[3.1.0]hexan-6-yl)-7-isopropoxyimidazo[1,2- | |
| | a]pyridine-6-carboxylic acid (Preparation 361) | |
| | R-NH₂: (6-difluoromethyl)pyridine-2-amine | |
| | prep-HPLC-A | |
| 254 | 2-(3-Oxabicyclo[3.1.0]hexan-6-yl)-7-isopropoxy-N-(6-methoxypyridin-2- | 5.0 mg, 12.34% yield as white |
| | yl)imidazo[1,2-a]pyridine-6-carboxamide | solid LCMS m/z = 409.1 |
| | | [M + H]⁺ ¹H NMR (500 MHz, |
| | 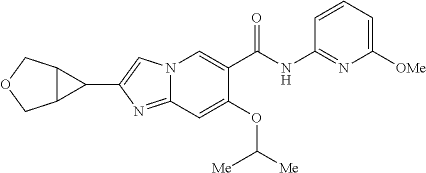 [SMILES: COc1cccc(NC(=O)c2cn3cc(C4C5COCC54)nc3cc2OC(C)C)n1] | MeOH-d₄) δ: 1.60 (d, 6H), 1.91 (t, 1H), 2.11 (d, 2H), 3.81 (d, 2H), 3.88 (s, 3H), 3.99 (d, 2H), 4.97-5.00 (m, 1H), 6.55 (d, 1H), 6.94 (s, 1H), 7.59 (s, 1H), 7.67 (t, 1H), 7.82 (d, 1H), 9.06 (s, 1H) |
| | RCO₂H: 2-(3-oxabicyclo[3.1.0]hexan-6-yl)-7-isopropoxyimidazo[1,2- | |
| | a]pyridine-6-carboxylic acid (Preparation 361) | |
| | R-NH₂: 6-methoxypyridin-2-amine | |
| | prep-HPLC-J | |
| 255 | 8-Chloro-7-isopropoxy-N-(1-methyl-2-oxo-1,2-dihydropyridin-3-yl)-2-(1- | 23.5 mg, 36% yield as a white |
| | methyl-2-oxabicyclo[2.1.1]hexan-4-yl)imidazo[1,2-a]pyridine-6-carboxamide | solid LCMS m/z = 457.1 |
| | | [M + H]⁺ ¹H NMR (500MHz, |
| | 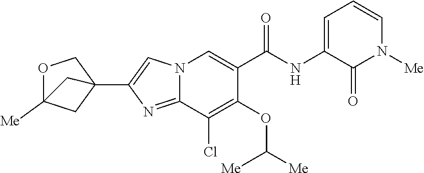 [SMILES: CC(C)Oc1c(C(=O)Nc2cccn(C)c2=O)cn2cc(C34COC(C)(C3)C4)nc2c1Cl] | MeOH-d₄) δ: 1.46 (d, 6H), 1.51 (s, 3H), 1.82-1.92 (m, 2H), 2.11-2.22 (m, 2H), 3.67 (s, 3H), 4.05 (s, 2H), 4.77-4.84 (m, 1H), 6.40 (t, 1H), 7.39 (d, 1H), 7.89 (s, 1H), 8.59 (dd, 1H), 9.08 (s, 1H) |
| | RCO₂H: 8-chloro-7-isopropoxy-2-(1-methyl-2-oxabicyclo[2.1.1]hexan-4- | |
| | yl)imidazo[1,2-a]pyridine-6-carboxylic acid (Preparation 315) | |
| | RNH₂: 3-amino-1-methyl-1,2-dihydropyridin-2-one. prep-HPLC-J | |
| 256 | 7-Cyclobutoxy-8-fluoro-2-(1-methyl-2-oxabicyclo[2.1.1]hexan-4-yl)-N-(6- | 50.8 mg, 89.1% yield as white |
| | methylpyrazolo[1,5-a]pyrimidin-3-yl)imidazo[1,2-a]pyridine-6-carboxamide | solid LCMS m/z = 477.0 |
| | | [M + H]⁺ ¹H NMR (500 MHz, |
| | 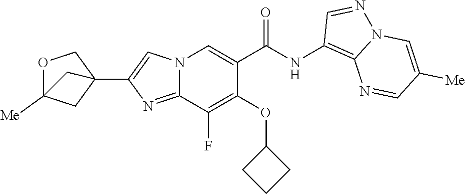 [SMILES: Cc1cnc2c(NC(=O)c3cn4cc(C56COC(C)(C5)C6)nc4c(F)c3OC3CCC3)cnn2c1] | CDCl₃) δ: 1.54 (s, 3H), 1.64- 1.70 (m, 1H), 1.93-1.84 (m, 1H), 1.98-2.00 (m, 2H), 2.11- 2.13 (m, 2H), 2.41 (s, 3H), 2.49-2.55 (m, 2H), 2.58-2.67 (m, 2H), 4.10 (s, 2H), 5.12- 5.18 (m, IH), 7.47 (d, 1H), 8.33 (d, 1H), 8.42 (s, 1H), 8.85 (s, 1H), 8.91 (s, 1H), 10.49 (s, 1H) |
| | RCO₂H: 7-cyclobutoxy-8-fluoro-2-(1-methyl-2-oxabicyclo[2.1.1]hexan-4- | |
| | yl)imidazo[1,2-a]pyridine-6-carboxylic acid (Preparation 321) | |
| | R-NH₂: 6-methylpyrazolo[1,5-a]pyrimidin-3-amine. prep-HPLC-J | |
| 257 | N-(3-cyano-2-fluorophenyl)-7-isopropoxy-2-(1-methyl-2-oxabicyclo | 8.8 mg, 12.82% yield |
| | [2.1.1]hexan-4-yl)imidazo[1,2-a]pyridine-6-carboxamide trifluoroacetate | LCMS m/z = 435.2 [M + H]⁺ |
| | | ¹H NMR (500 MHz, DMSO- |
| | 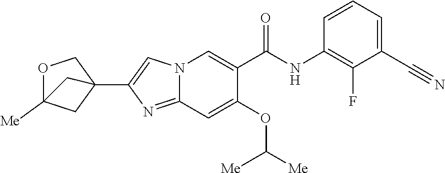 [SMILES: CC(C)Oc1cc2nc(C34COC(C)(C3)C4)cn2cc1C(=O)Nc1cccc(C#N)c1F] | d₆) δ: 1.43-1.51 (m, 9H), 1.81- 1.90 (m, 2H), 2.07-2.14 (m, 2H), 3.93 (s, 2H), 5.02-5.14 (m, 1H), 7.28 (s, 1H), 7.49 (t, 1H), 7.76 (t, 1H), 8.02 (br s, 1H), 8.52 (br s, 1H), 9.25 (s, 1H), 10.39 (s, 1H) |
| | RCO₂H: 7-isopropoxy-2-(1-methyl-2-oxabicyclo[2.1.1]hexan-4- | |
| | yl)imidazo[1,2-a]pyridine-6-carboxylic acid (Preparation 78) | |
| | R-NH₂: 3-amino-2-fluorobenzonitrile | |
| | Prep-HPLC-D | |
| 258 | 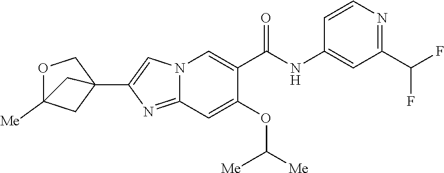 [SMILES: CC(C)Oc1cc2nc(C34COC(C)(C3)C4)cn2cc1C(=O)Nc1ccnc(C(F)F)c1] | 11 mg, 19.66% yield LCMS m/z = 443.0 [M + H]⁺ ¹H NMR (500 MHz, DMSO- d₆) δ: 1.39 (d, 6H), 1.45 (s, 3H), 1.84 (brd, 2H), 2.11 (br s, 2H), 3.93 (s, 2H), 4.85-5.04 (m, 1H), 6.84-7.11 (m, 1H), 7.20 (d, 1H), 7.76 (br d, 1H), 7.98 (br s, 1H), 8.06 (s, 1H), 8.63 (d, 1H), 9.07 (br s, 1H), 10.90 (br s, 1H) |
| | N-(2-(difluoromethyl)pyridin-4-yl)-7-isopropoxy-2-(1-methyl-2- | |
| | oxabicyclo[2.1.1]hexan-4-yl)imidazo[1,2-a]pyridine-6-carboxamide | |
| | RCO₂H: 7-isopropoxy-2-(1-methyl-2-oxabicyclo[2.1.1]hexan-4- | |
| | yl)imidazo[1,2-a]pyridine-6-carboxylic acid (Preparation 78) | |
| | R-NH₂: 2-(difluoromethyl)pyridine-4-amine | |
| 259 | 7-Isopropoxy-2-(1-methyl-2-oxabicyclo[2.1.1]hexan-4-yl)-N-(6- | 41.1 mg, 39.98% yield |
| | methylpyridin-2-yl)imidazo[1,2-a]pyridine-6-carboxamide | LCMS m/z = 407.3 [M + H]⁺ |
| | | 1H NMR (500 MHz, DMSO- |
| | 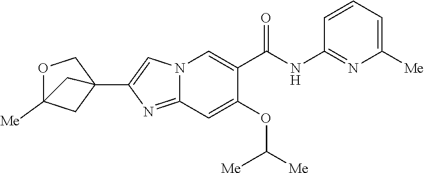 [SMILES: Cc1cccc(NC(=O)c2cn3cc(C45COC(C)(C4)C5)nc3cc2OC(C)C)n1] | d₆) δ: 1.43 (s, 3H), 1.46 (d, 6H), 1.73-1.79 (m, 2H), 1.94- 2.05 (m, 2H), 2.42 (s, 3H), 3.88 (s, 2H), 4.95 (dt, 1H), 7.05 (d, 1H), 7.16 (s, 1H), 7.75 (t, 1H), 7.81 (s, 1H), 8.03 (br d, 1H), 9.12 (s, 1H), 10.62 (s, 1H) |
| | RCO₂H: 7-isopropoxy-2-(1-methyl-2-oxabicyclo[2.1.1]hexan-4- | |
| | yl)imidazo[1,2-a]pyridine-6-carboxylic acid (Preparation 78) | |
| | R-NH₂: 6-methylpyridin-2-amine | |
| | Prep-HPLC-F | |
| 260 | N-(6-(dimethylamino)pyridin-2-yl)-7-isopropoxy-2-(1-methyl-2- | 23.90 mg, 34.7% yield |
| | oxabicyclo[2.1.1]hexan-4-yl)imidazo[1,2-a]pyridine-6-carboxamide | LCMS m/z = 436.3 [M + H]⁺ |
| | 2,2,2-trifluoroacetate | ¹H NMR (500 MHz, DMSO- |
| | | d₆) δ: 1.45 (s, 3H), 1.50 (d, |
| | 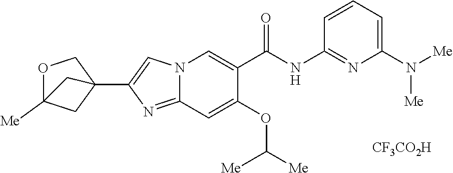 [SMILES: CC(C)Oc1cc2nc(C34COC(C)(C3)C4)cn2cc1C(=O)Nc1cccc(N(C)C)n1 ; "CF3CO2H"] | 6H), 1.82-1.88 (m, 2H), 2.05- 2.15 (m, 2H), 3.35 (s, 6H), 3.93 (s, 2H), 5.11 (br s, 1H), 6.46 (br d, 1H), 7.27 (s, 1H), 7.43 (br d, 1H), 7.57 - 7.63 (m, 1H), 8.03 (br s, 1H), 9.28 (br s, 1H), 10.34 (br s, 1H) |
| | RCO₂H: 7-isopropoxy-2-(1-methyl-2-oxabicyclo[2.1.1]hexan-4- | |
| | yl)imidazo[1,2-a]pyridine-6-carboxylic acid (Preparation 78) | |
| | R-NH₂: N2,N2-dimethylpyridine-2,6-diamine | |
| | Prep-HPLC-D | |
| 261 | 7-Isopropoxy-N-(1-methyl-6-oxo-1,6-dihydropyrimidin-5-yl)-2-(1- | 13.70 mg, 26.88% yield |
| | methyl-2-oxabicyclo[2.1.1]hexan-4-yl)imidazo[1,2-a]pyridine-6- | LCMS m/z = 424.4 [M + H]⁺ |
| | carboxamide 2,2,2-trifluoroacetate | ¹H NMR (500 MHz, DMSO- |
| | | d₆) δ: 1.45 (s, 3H), 1.55 (d, |
| | 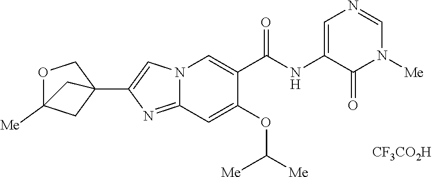 [SMILES: CC(C)Oc1cc2nc(C34COC(C)(C3)C4)cn2cc1C(=O)Nc1cncn(C)c1=O ; "CF3CO2H"] | 6H), 1.82-1.87 (m, 2H), 2.11 (br d, 2H) 3.55 (s, 3H), 3.93 (s, 2H), 5.18-5.22 (m, 1H), 7.34 (s, 1H), 8.07 (br s, 1H), 8.33 (s, 1H), 8.99 (s, 1H), 9.40 (br s, 1H), 10.48 (s, 1H) |
| | RCO₂H: 7-isopropoxy-2-(1-methyl-2-oxabicyclo[2.1.1]hexan-4- | |
| | yl)imidazo[1,2-a]pyridine-6-carboxylic acid (Preparation 78) | |
| | R-NH₂: 5-amino-3-methyl-pyrimidin-4-one | |
| | Prep-HPLC-D | |
| 262 | 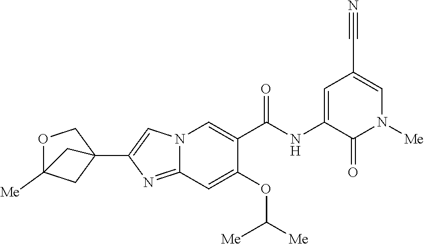 [SMILES: CC(C)Oc1cc2nc(C34COC(C)(C3)C4)cn2cc1C(=O)Nc1cc(C#N)cn(C)c1=O] | 14.6 mg, 34.41% yield LCMS m/z = 448.2 [M + H]⁺ ¹H NMR (500 MHz, DMSO- d) δ: 1.45 (s, 3H), 1.54 (d, 6H), 1.80-1.91 (m, 2H), 2.10 (br d, 2H), 3.60 (s, 3H), 3.92 (s, 2H), 5.18 (br d, 1H), 7.33 (s, 1H), 8.05 (br s, 1H), 8.45 (d, 1H), 9.37 (br s, 1H), 10.72 (s, 1H) |
| | N-(5-cyano-1-methyl-2-oxo-1,2-dihydropyridin-3-yl)-7-isopropoxy-2-(1- | |
| | methyl-2-oxabicyclo[2.1.1]hexan-4-yl)imidazo[1,2-a]pyridine-6-carboxamide | |
| | RCO₂H: 7-isopropoxy-2-(1-methyl-2-oxabicyclo[2.1.1]hexan-4-yl)imidazo[1,2- | |
| | a]pyridine-6-carboxylic acid (Preparation 78) | |
| | R-NH₂: 5-amino-1-methyl-6-oxo-1,6-dihydropyridine-3-carbonitrile | |
| 263 | 7-Isopropoxy-2-(1-methyl-2-oxabicyclo[2.1.1]hexan-4-yl)-N-(5- | 10.10 mg, 17.89% yield |
| | methylpyrazolo[1,5-a]pyrimidin-3-yl)imidazo[1,2-a]pyridine-6-carboxamide | LCMS m/z = 447.0 [M + H]⁺ |
| | | ¹H NMR (500 MHz, DMSO- |
| | 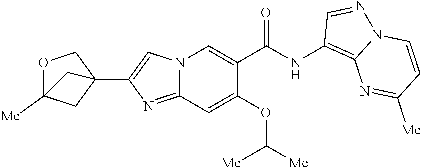 [SMILES: Cc1ccn2ncc(NC(=O)c3cn4cc(C56COC(C)(C5)C6)nc4cc3OC(C)C)c2n1] | d₆) δ 1.45 (s, 3H), 1.54 (d, 6H), 1.85 (br d, 2H), 2.11 (br s, 2H),2.35(s, 3H), 3.94 (s, 2H) 4.99-5.20 (m, 1H), 7.30 (s, 1H), 8.05 (br s, 1H), 8.49 (d, 1H), 8.66 (s, 1H), 8.96 (d, 1H), 9.29 (s, 1H), 10.50 (s, 1H) |
| | RCO₂H: 7-isopropoxy-2-(1-methyl-2-oxabicyclo[2.1.1]hexan-4- | |
| | yl)imidazo[1,2-a]pyridine-6-carboxylic acid (Preparation 78) | |
| | R-NH₂: 6-methylpyrazolo[1,5-a]pyrimidin-3-amine | |
| 264 | N-(5-chloropyrazolo[1,5-a]pyrimidin-3-yl)-7- | 12.80 mg, 21.7% yield |
| | isopropoxy-2-(1-methyl-2- | LCMS m/z = 467.1 [M + H]⁺ |
| | oxabicyclo[2.1.1]hexan-4-yl)imidazo[1,2- | ¹H NMR (500 MHz, DMSO- |
| | a]pyridine-6-carboxamide | d₆) δ: 1.44 (s, 3H), 1.56 (d, |
| | | 6H), 1.77 (dd, 2H), 1.96-2.04 |
| | 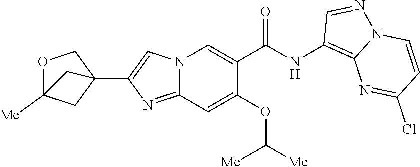 [SMILES: CC(C)Oc1cc2nc(C34COC(C)(C3)C4)cn2cc1C(=O)Nc1cnn2ccc(Cl)nc12] | (m, 2H), 3.89 (s, 2H), 5.04 (spt, 1H), 7.16 (d, 1H), 7.23 (s, 1H), 7.85 (s, 1H), 8.79 (s, 1H), 9.16 (d, 1H), 9.20 (s, 1H), 10.49 (s, 1H) |
| | RCO₂H: 7-isopropoxy-2-(1-methyl-2-oxabicyclo[2.1.1]hexan-4- | |
| | yl)imidazo[1,2-a]pyridine-6-carboxylic acid (Preparation 78) | |
| | R-NH₂: 5-chloropyrazolo[1,5-a]pyrimidin-3-amine | |
| 265 | 2-Chloro-N-(6-(difluoromethyl)pyridin-2-yl)-7- | LCMS m/z = 380.0 [M + H]⁺ |
| | isopropoxyimidazo[1,2-a]pyridine-6-carboxamide | ¹H NMR (400 MHz, DMSO- |
| | | d₆) δ: 1.43 (d, 6H), 4.96 (quin, |
| | 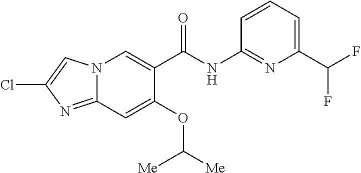 [SMILES: CC(C)Oc1cc2nc(Cl)cn2cc1C(=O)Nc1cccc(C(F)F)n1] | 1H), 6.73-7.07 (m, 1H), 7.18 (s, 1H), 7.48 (d, 1H), 8.01 (s, 1H), 8.08 (t, 1H), 8.26-8.45 (m, 1H), 9.08 (s, 1H), 10.89 (s, 1H). |
| | RCO₂H: 2-chloro-7-isopropoxyimidazo[1,2-a]pyridine-6-carboxylic acid | |
| | (Preparation 306) | |
| | R-NH₂: | |
| 266 | 2-(tert-Butyl)-7-cyclobutoxy-N-(pyrazolo[1,5-a]pyrimidin-3- | LCMS m/z = 405.0 [M + H]⁺ |
| | yl)imidazo[1,2-a]pyridine-6-carboxamide trifluoroacetate | ¹H NMR (500 MHz, DMSO- |
| | | d₆) δ: 1.31 (s, 9H), 1.72-1.87 |
| | 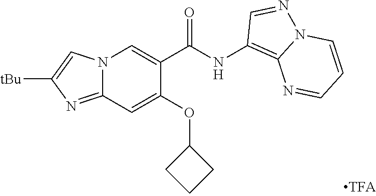 [SMILES: CC(C)(C)c1cn2cc(C(=O)Nc3cnn4cccnc34)c(OC3CCC3)cc2n1] | (m, 1H), 1.90-2.03 (m, 1H), 2.39-2.48 (m, 4H), 3.17 (d, 1H), 5.01-5.13 (m, 1H), 6.94 (s, 1H), 7.07 (dd, 1H), 7.71 (s, 1H), 8.56 (dd, 1H), 8.77 (s, 1H), 9.10 (dd, 1H), 9.17 (s, 1H), 10.46 (s, 1H). |
| | RCO₂H: 2-(tert-butyl)-7-cyclobutoxyimidazo[1,2-a]pyridine-6-carboxylic | |
| | acid (Preparation 323) | |
| | R-NH₂: pyrazolo[1,5-a]pyrimin-3-amine. prep-HPLC-D | |
| 267 | 2-(tert-Butyl)-7-cyclobutoxy-N-(6-(difluoromethyl)pyridin-2- | LCMS m/z = 415.0 [M + H]⁺ |
| | yl)imidazo[1,2-a]pyridine-6-carboxamide | ¹H NMR (500 MHz, DMSO- |
| | | d₆) δ: 1.39 (s, 9H), 1.70-1.83 |
| | 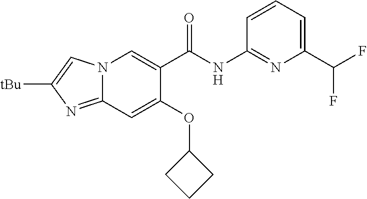 [SMILES: CC(C)(C)c1cn2cc(C(=O)Nc3cccc(C(F)F)n3)c(OC3CCC3)cc2n1] | (m, 1H), 1.84-1.98 (m, 1H), 2.21-2.25 (m, 2H), 2.54-2.57 (m, 1H), 5.07-5.11 (m, 1H), 6.78-7.09 (m, 2H), 7.52 (d, 1H), 7.95 (s, 1H), 8.11 (t, 1H), 8.36 (br s, 1H), 9.15 (s, 1H), 11.15 (s, 1H). |
| | RCO₂H: 2-(tert-butyl)-7-cyclobutoxyimidazo[1,2-a]pyridine-6-carboxylic | |
| | acid (Preparation 323) | |
| | R-NH₂: 6-(difluoromethyl)pyridine-2-amine. prep-HPLC-J | |
| 268 | 7-((4-Oxaspiro[2.4]heptan-6-yl)oxy)-2-(tert-butyl)-N-(6- | LCMS m/z = 457.0 [M + H]⁺ |
| | (difluoromethyl)pyridin-2-yl)imidazo[1,2-a]pyridine-6-carboxamide | ¹H NMR (500 MHz, MeOH- |
| | | d₄) δ: 0.58-0.94 (m, 5H), 1.38 |
| | 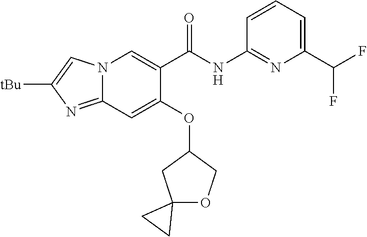 [SMILES: CC(C)(C)c1cn2cc(C(=O)Nc3cccc(C(F)F)n3)c(OC3COC4(CC4)C3)cc2n1] | (s, 9H), 2.36 (d, 1H), 2.60- 2.73 (m, 1H), 4.23 (d, 1H), 4.29-4.38 (m, 1H), 5.46 (s, 1H), 6.43-6.74 (m, 1H), 6.87 (s, 1H), 7.43 (d, 1H), 7.60 (s, 1H), 7.99 (t, 1H), 8.44 (d, 1H), 9.10 (s, 1H). |
| | RCO₂H: 7-((4-oxaspiro[2.4]heptan-6-yl)oxy)-2-(tert-butyl)imidazo[1,2- | |
| | a]pyridine-6-carboxylic acid (Preparation 324) | |
| | R-NH₂: 6-(difluoromethyl)pyridin-2-amine | |
| | prep-HPLC-D | |
| 269 | N-(6-(Difluoromethyl)pyridin-2 -yl)-8-fluoro-2-(1-(fluoromethyl)-2- | LCMS m/z = 478.9 [M + H]⁺ |
| | oxabicyclo[2.1.1]hexan-4-yl)-7-isopropoxyimidazo[1,2-a]pyridine-6- | ¹H NMR (500 MHz, DMSO- |
| | carboxamide | d₆) δ: 1.34 (d, 6H), 1.39 (s, |
| | | 1H), 1.90 (dd, 2H), 2.20 (dd, |
| | 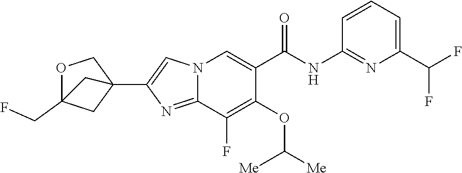 [SMILES: CC(C)Oc1c(C(=O)Nc2cccc(C(F)F)n2)cn2cc(C34COC(CF)(C3)C4)nc2c1F] | 2H), 3.17 (s, 1H), 4.00 (s, 2H), 4.58-4.69 (m, 2H), 4.76 (s, 1H), 6.79-7.05 (m, 1H), 7.50 (d, 1H), 8.03 (d, 1H), 8.10 (t, 1H), 8.36 (br d, 1H), 8.95 (s, 1H), 11.02 (s, 1H). |
| | RCO₂H: 8-fluoro-2-(1-(fluoromethyl)-2-oxabicyclo[2.1.1]hexan-4-yl)-7- | |
| | isopropoxyimidazo[1,2-a]pyridine-6-carboxylic acid (Preparation 322) | |
| | R-NH₂: 6-(difluoromethyl)pyridin-2-amine | |
| | prep-HPLC-D | |
| 270 | 8-Fluoro-2-(1-(fluoromethyl)-2-oxabicyclo[2.1.1]hexan-4-yl)-7-isopropoxy-N- | LCMS m/z = 482.9 [M + H]⁺ |
| | (6-methylpyrazolo[1,5-a]pyrimidin-3-yl)imidazo[1,2-a]pyridine-6-carboxamide | ¹H NMR (500 MHz, DMSO- |
| | | d₆) δ: 1.42 (d, 6H), 1.90 (dd, |
| | 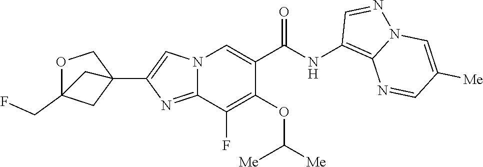 [SMILES: Cc1cnc2c(NC(=O)c3cn4cc(C56COC(CF)(C5)C6)nc4c(F)c3OC(C)C)cnn2c1] | 2H), 2.16-2.23 (m, 2H), 2.35 (s, 3H), 4.00 (s, 2H), 4.63-4.79 (m, 3H), 8.06 (d, 1H), 8.49 (d, 1H), 8.64 (s, 1H), 8.96 (d, 1H), 9.01 (s, 1H), 10.46 (s, 1H). |
| | RCO₂H: 8-fluoro-2-(1-(fluoromethyl)-2-oxabicyclo[2.1.1]hexan-4-yl)-7- | |
| | isopropoxyimidazo[1,2-a]pyridine-6-carboxylic acid (Preparation 322) | |
| | R-NH₂: 6-methylpyrazolo[1,5-a]pyrimidin-3-amine. prep-HPLC-J | |
| 271 | 8-Ethoxy-N-(5-fluoro-2-methoxypyridin-3-yl)-2-(tetrahydro-2H-pyran-3- | 117.2 mg, 58.7% |
| | yl)imidazo[1,2-a]pyrazine-6-carboxamide | LCMS m/z = 416.3 [M + H]⁺ |
| | | ¹H NMR (400 MHz, CDCl₃) |
| | 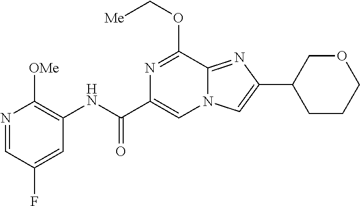 [SMILES: CCOc1nc(C(=O)Nc2cc(F)cnc2OC)cn2cc(C3CCCOC3)nc12] | δ: 1.66 (t, 3H), 1.74-1.84 (m, 2H), 1.88-2.03 (m, 1H), 2.21 (br dd, 1H), 3.15-3.27 (m, 1H), 3.55-3.65 (m, 1H), 3.69 (dd, 1H), 3.89-3.99 (m, 1H), 4.06- 4.11 (m, 3H),4.19(dd, 1H), 4.73-4.85 (m, 2H), 7.62 (s, 1H), 7.77 (d, 1H), 8.58-8.73 (m, 2H), 10.17 (s, 1H). |
| | RCO₂H: 8-ethoxy-2-(tetrahydro-2H-pyran-3-yl)imidazo[1,2-a]pyrazine-6- | |
| | carboxylic acid (Preparation 131). | |
| | R-NH₂: prep-HPLC-J | |
| 272 | 8-Ethoxy-N-(5-fluoro-1-methyl-2-oxo-1,2-dihydropyridin-3-yl)-2- | 75.3 mg, 37.7% |
| | (tetrahydro-2H-pyran-3-yl)imidazo[1,2-a]pyrazine-6-carboxamide | LCMS m/z = 416.2 [M + H]⁺ |
| | | ¹H NMR (400 MHz, CDCl₃) |
| | 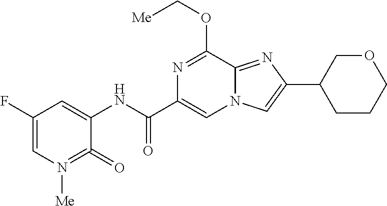 | δ: 1.65 (t, 3H), 1.72-1.77 (m, 2H), 1.94-2.00 (m, 1H), 2.18- 2.28 (m, 1H), 3.15-3.26 (m, 1H), 3.55-3.75 (m, 5H), 3.90- 3.99 (m, 1H), 4.14-4.22 (m, 1H), 4.77-4.86 (m, 2H), 7.02 (dd, 1H), 7.60 (s, 1H), 8.56- 8.65 (m, 2H), 10.68 (s, 1H). |
| | RCO₂H: 8-ethoxy-2-(tetrahydro-2H-pyran-3-yl)imidazo[1,2-a]pyrazine-6- | |
| | carboxylic acid (Preparation 131). | |
| | R-NH₂: prep-HPLC-J | |
| 273 | 8-Ethoxy-N-(2-methoxypyridin-3-yl)-2-(tetrahydrofuran-3- | 97.7 mg, 61.3% |
| | yl)imidazo[1,2-a]pyrazine-6-carboxamide | LCMS m/z = 384.2 [M + H]⁺ |
| | | ¹H NMR (400 MHz, CDCl₃) |
| | 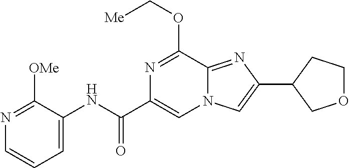 [SMILES: CCOc1nc(C(=O)Nc2cccnc2OC)cn2cc(C3CCOC3)nc12] | δ: 1.64 (t, 3H), 2.13-2.29 (m, 1H), 2.35-2.50 (m, 1H), 3.70 (quin, 1H), 3.89-3.98 (m, 2H), 3.98-4.10 (m, 4H),4.18 (dd, 1H), 4.77 (q, 2H), 6.95 (dd, 1H), 7.51-7.61 (m, 1H), 7.89 (dd, 1H), 8.55-8.66 (m, 1H), 8.73 (dd, 1H), 10.12 (s, 1H). |
| | RCO₂H: 8-ethoxy-2-(tetrahydrofuran-3-yl)imidazo[1,2-a]pyrazine-6- | |
| | carboxylic acid (Preparation 366). | |
| | R-NH₂: prep-HPLC-J | |
| 274 | N-(6-(difluoromethyl)pyridin-2-yl)-8-ethoxy-2-(1-methyl-2- | 49.6 mg, 57.8% LCMS m/z = |
| | oxabicyclo[2.2.1]heptan-4-yl)imidazo[1,2-a]pyrazine-6-carboxamide | 444.2 [M + H]⁺ ¹H NMR (400 |
| | | MHz, CDCl₃) δ: H23 1.43- |
| | 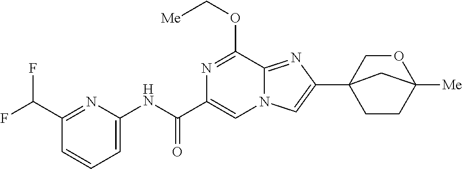 [SMILES: CCOc1nc(C(=O)Nc2cccc(C(F)F)n2)cn2cc(C34CCC(C)(C3)OC4)nc12] | 1.52 (m, 4H), 1.65 (t, 3H), 1.79-1.91 (m, 3H), 1.98 (d, 1H), 2.02-2.10 (m, 1H), 2.10- 2.17 (m, 1H), 2.17-2.27 (m, 1H), 4.02 (d, 1H),4.14 (dd, 1H), 4.81 (q, 2H), 6.43-6.70 (m, 1H), 7.44 (d, 1H), 7.51- 7.62 (m, 1H), 7.93 (t, 1H), 8.52 (d, 1H), 8.64-8.73 (m, 1H), 9.90-10.04 (m, 1H). |
| | RCO₂H: 8-ethoxy-2-(1-methyl-2-oxabicyclo[2.2.1]heptan-4- | |
| | yl)imidazo[1,2-a]pyrazine-6-carboxylic acid (Preparation 335). | |
| | R-NH₂: prep-HPLC-J | |
| 275 | N-(2-methoxypyridin-3-yl)-2-(1-methyl-2-oxabicyclo[2.2.1]heptan-4- | 36.3 mg, 23.4% |
| | yl)-8-propoxyimidazo[1,2-a]pyrazine-6-carboxamide trifluoroacetate | LCMS m/z = 438.3 [M + H]⁺ |
| | | ¹H NMR (600 MHz, CDCl₃) |
| | 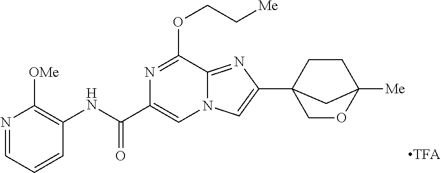 [SMILES: CCCOc1nc(C(=O)Nc2cccnc2OC)cn2cc(C34CCC(C)(C3)OC4)nc12] | δ: 1.14 (t, 3H), 1.55-1.61 (m, 3H), 1.84-1.97 (m, IH), 1.97- 2.06 (m, 3H), 2.08-2.18 (m, 2H), 2.18-2.29 (m, 2H), 4.09- 4.16 (m, 2H), 4.20-4.25 (m, 3H), 4.66 (t, 2H), 7.16 (dd, 1H), 7.66-7.76 (m, 1H), 8.06 (dd, 1H), 8.75-8.84 (m, 1H), 8.92 (dd, 1H), 10.05 (br s, 1H). |
| | RCO₂H: 2-(1-methyl-2-oxabicyclo[2.2.1]heptan-4-yl)-8- | |
| | propoxyimidazo[1,2-a]pyrazine-6-carboxylic acid (Preparation 368) | |
| | R-NH₂: prep-HPLC-D | |
| 276 | 8-Ethoxy-N-(2-methoxypyridin-3-yl)-2-(1-methyl-2-oxabicyclo[2.2.1]heptan- | 28.8 mg, 39.5% |
| | 4-yl)imidazo[1,2-a]pyrazine-6-carboxamide trifluoroacetate | LCMS m/z = 424.4 [M + H]⁺ |
| | | ¹H NMR (600 MHz, CDCl₃) |
| | 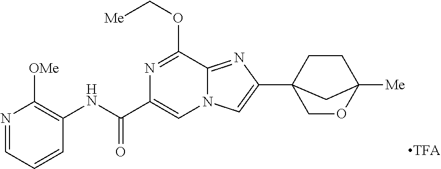 [SMILES: CCOc1nc(C(=O)Nc2cccnc2OC)cn2cc(C34CCC(C)(C3)OC4)nc12] | δ: 1.47-1.59 (m, 3H), 1.62 (t, 3H), 1.86-1.96 (m, 1H), 1.96- 2.07 (m, 1H), 2.07-2.18 (m, 2H), 2.18-2.30 (m, 2H), 4.08-4.17 (m, 2H), 4.19-4.26 (m, 3H), 4.69-4.83 (m, 2H), 7.15 (dd, 1H), 7.66-7.76 (m, 1H), 8.06 (dd, 1H), 8.72-8.84 (m, 1H), 8.84-8.95 (m, 1H), 10.00-10.17 (m, 1H). |
| | RCO₂H: 8-ethoxy-2-(1-methyl-2-oxabicyclo[2.2.1]heptan-4-yl)imidazo[1,2- | |
| | a]pyrazine-6-carboxylic acid (Preparation 335) | |
| | R-NH₂: prep-HPLC-D | |
| 277 | 8-Ethoxy-N-(5-fluoro-1-methyl-2-oxo-1,2-dihydropyridin-3-yl)-2-(1-methyl- | 23.1 mg, 46% |
| | 2-oxabicyclo[2.2.1]heptan-4-yl)imidazo[1,2-a]pyrazine-6-carboxamide | LCMS m/z = 442.2 [M + H]⁺ |
| | trifluoroacetate | ¹H NMR (500 MHz, DMSO- |
| | | d₆) δ: 1.39 (s, 3H), 1.52 (t, |
| | 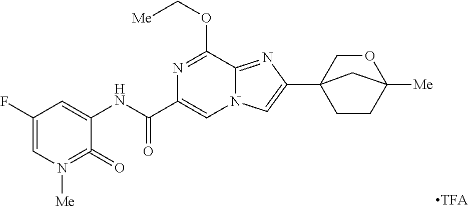 | 3H), 1.63-1.75 (m, IH), 1.75- 1.86 (m, 2H), 1.86-1.93 (m, 1H), 1.98 (dddd, 1H), 2.03- 2.14 (m, 1H), 3.55 (s, 3H), 3.81 (d, 1H), 3.93 (dd, 1H), 4.65 (q, 2H), 7.76 (dd, 1H), 8.05-8.16 (m, 1H), 8.39 (dd, 1H), 8.96 (s, 1H), 10.57 (s, 1H). |
| | RCO₂H: 8-ethoxy-2-(1-methyl-2-oxabicyclo[2.2.1]heptan-4-yl)imidazo[1,2- | |
| | a]pyrazine-6-carboxylic acid (Preparation 335) | |
| | R-NH₂: prep-HPLC-D | |
| 278 | 8-Ethoxy-2-(1-methyl-2-oxabicyclo[2.2.1]heptan-4-yl)-N-(pyrazolo[1,5- | 22.2 mg, 44.9% LCMS m/z = |
| | a]pyridin-7-yl)imidazo[1,2-a]pyrazine-6-carboxamide trifluoroacetate | 433.2 [M + H]⁺ ¹H NMR (500 |
| | | MHz, DMSO-d₆) δ: 1.40 (s, |
| | 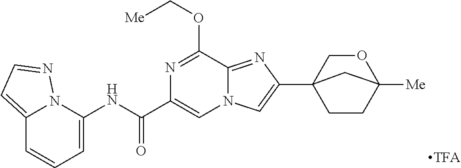 [SMILES: CCOc1nc(C(=O)Nc2cccc3ccnn23)cn2cc(C34CCC(C)(C3)OC4)nc12] | 3H), 1.59 (t, 3H), 1.67-1.77 (m, 1H), 1.77-1.88 (m, 2H), 1.88-1.95 (m, 1H), 2.00 (dddd, 1H), 2.05-2.14 (m, 1H), 3.83 (d, 1H), 3.95 (dd, 1H), 4.79 (q, 2H), 6.75 (d, 1H), 7.37 (dd, 1H), 7.48-7.58 (m, 1H), 7.77 (dd, 1H), 8.14 (s, 1H), 8.17 (d, 1H), 9.07 (s, 1H), 11.64 (s, 1H). |
| | RCO₂H: 8-ethoxy-2-(1-methyl-2-oxabicyclo[2.2.1]heptan-4-yl)imidazo[1,2- | |
| | a]pyrazine-6-carboxylic acid (Preparation 335) | |
| | R-NH₂: prep-HPLC-D | |
| 279 | 2-Cyclopropyl-8-ethoxy-N-(2-methoxypyridin-3-yl)imidazo[1,2-a]pyrazine- | 18.2 mg, 19.2% |
| | 6-carboxamide trifluoroacetate | LCMS m/z = 354.2 [M + H]⁺ |
| | | ¹H NMR (500 MHz, DMSO- |
| | 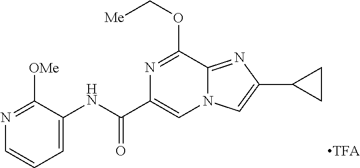 [SMILES: CCOc1nc(C(=O)Nc2cccnc2OC)cn2cc(C3CC3)nc12] | d₆) δ: 0.80-0.92 (m, 2H), 0.92- 1.02 (m, 2H), 1.45-1.56 (m, 3H), 2.06-2.17 (m, 1H), 3.97- 4.07 (m, 3H), 4.66 (q, 2H), 7.08 (dd, 1H), 7.94 (dd, 1H), 8.04 (s, 1H), 8.61 (dd, 1H), 8.92 (s, 1H), 10.12 (s, 1H). |
| | RCO₂H: 2-cyclopropyl-8-ethoxyimidazo[1,2-a]pyrazine-6-carboxylic acid | |
| | (Preparation 341) | |
| | R-NH₂: prep-HPLC-D | |
| 280 | 2-Cyclopropyl-7-isopropoxy-N-(6-methylpyrazolo[1,5-a]pyrimidin-3- | 22.1 mg, 20.9% |
| | yl)imidazo[1,2-a]pyridine-6-carboxamide trifluoroacetate | LCMS m/z = 391.3 [M + H]⁺ |
| | | ¹H NMR (500 MHz, DMSO- |
| | 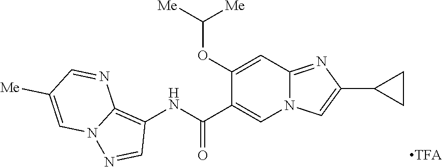 [SMILES: Cc1cnc2c(NC(=O)c3cn4cc(C5CC5)nc4cc3OC(C)C)cnn2c1] | d₆) δ: 0.84-0.92 (m, 2H), 1.02- 1.15 (m, 2H), 1.52 (d, 6H), 2.09-2.20 (m, 1H), 2.31-2.40 (m, 3H), 5.12 (spt, 1H), 7.27 (s, 1H), 7.94 (s, 1H), 8.48 (d, 1H), 8.65 (s, 1H), 8.96 (d, 1H), 9.25 (s, 1H), 10.48 (s, 1H). |
| | RCO₂H: 2-cyclopropyl-7-isopropoxyimidazo[1,2-a]pyridine-6-carboxylic | |
| | acid (Preparation 299). | |
| | R-NH₂: prep-HPLC-D | |
| 281 | 8-Cyclopropoxy-2-cyclopropyl-N-(1-methyl-1H-pyrazol-3- | 11 mg, 22.8% |
| | yl)imidazo[1,2-a]pyrazine-6-carboxamide | LCMS m/z = 339.2 [M + H]⁺ |
| | | ¹H NMR (500 MHz, DMSO- |
| | 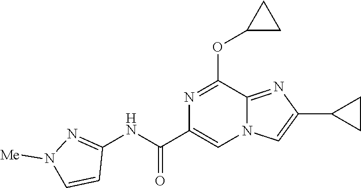 [SMILES: Cn1ccc(NC(=O)c2cn3cc(C4CC4)nc3c(OC3CC3)n2)n1] | d₆) δ: 0.75-0.90 (m, 6H), 0.93- 1.04 (m, 2H), 2.09 (tt, 1H), 3.80 (s, 3H), 4.81-4.95 (m, 1H), 6.61 (d, 1H), 7.66 (d, 1H), 8.03 (s, 1H), 8.90 (s, 1H), 10.15 (s, 1H). |
| | RCO₂H: 8-cyclopropoxy-2-cyclopropylimidazo[1,2-a]pyrazine-6-carboxylic | |
| | acid (Preparation 344) | |
| | R-NH₂: prep-HPLC-J | |
| 282 | N-(1-(cyclopropylmethyl)-1H-pyrazol-3-yl)-8-ethoxy-2-(1-methyl-2- | 82.3 mg, 85.5% |
| | oxabicyclo[2.2.1]heptan-4-yl)imidazo[1,2-a]pyrazine-6-carboxamide | LCMS m/z = 437.3 [M + H]⁺ |
| | | ¹H NMR (500 MHz, DMSO- |
| | 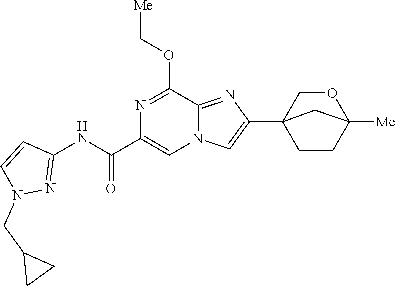 [SMILES: CCOc1nc(C(=O)Nc2ccn(CC3CC3)n2)cn2cc(C34CCC(C)(C3)OC4)nc12] | d₆) δ: 0.32-0.40 (m, 2H), 0.49- 0.63 (m, 2H), 1.20-1.30 (m, 1H), 1.33-1.42 (m, 3H), 1.47 (t, 3H), 1.66-1.76 (m, 1H), 1.76-1.86 (m, 2H), 1.86-1.94 (m, 1H), 1.94-2.02 (m, 1H), 2.02-2.13 (m, 1H), 3.77-3.83 (m, 1H), 3.89-3.96 (m, 3H), 4.72 (q, 2H), 6.62 (d, 1H), 7.73 (d, 1H), 8.09 (s, 1H), 8.88 (s, 1H), 10.18 (s, 1H). |
| | RCO₂H: 8-ethoxy-2-(1-methyl-2-oxabicyclo[2.2.1]heptan-4-yl)imidazo[1,2- | |
| | a]pyrazine-6-carboxylic acid (Preparation 335). | |
| | R-NH₂: prep-HPLC-J | |
| 283 | 2-Cyclopropyl-7-isopropoxy-N-(2-methoxypyridin-3-yl)imidazo[1,2- | 18.6 mg, 38.5% |
| | a]pyrimidine-6-carboxamide | LCMS m/z = 368.2 [M + H]⁺ |
| | | ¹H NMR (500 MHz, DMSO- |
| | 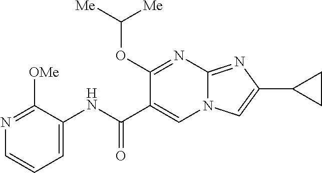 [SMILES: COc1ncccc1NC(=O)c1cn2cc(C3CC3)nc2nc1OC(C)C] | d₆) δ: 0.75-0.88 (m, 2H), 0.88- 0.99 (m, 2H), 1.52 (d, 6H), 1.95-2.08 (m, 1H), 3.96-4.10 (m, 3H), 5.53-5.67 (m, 1H), 7.08 (dd, 1H), 7.68 (s, 1H), 7.94 (dd, 1H), 8.73 (dd, 1H), 9.45 (s, 1H), 10.17 (s, 1H). |
| | RCO₂H: 2-cyclopropyl-7-isopropoxyimidazo[1,2-a]pyrimidine-6-carboxylic | |
| | acid (Preparation 357) | |
| | R-NH₂: prep-HPLC-J | |
| 284 | N-(5-fluoro-1-methyl-1H-pyrazol-3-yl)-7-isopropoxy-2-(1-methyl-2- | 24.16 mg, 14.8% |
| | oxabicyclo[2.2.1]heptan-4-yl)imidazo[1,2-a]pyridine-6-carboxamide | LCMS m/z = 428.2 [M + H]⁺ |
| | trifluoroacetate | ¹H NMR (400 MHz, MeOH- |
| | | d₄) δ: 1.45-1.53 (m, 3H), 1.57 |
| | 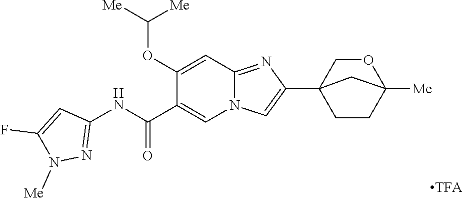 [SMILES: CC(C)Oc1cc2nc(C34CCC(C)(C3)OC4)cn2cc1C(=O)Nc1cc(F)n(C)n1] | (d, 6H), 1.83-2.01 (m, 2H), 2.03-2.11 (m, 2H), 2.11-2.30 (m, 2H), 3.70 (d, 3H), 3.93- 4.02 (m, 1H), 4.02-4.09 (m, 1H), 5.02-5.14 (m, 1H), 6.33 (d, 1H), 7.32 (s, 1H), 7.96 (s, 1H), 9.14 (s, 1H). |
| | RCO₂H: 7-isopropoxy-2-(1-methyl-2-oxabicyclo[2.2.1]heptan-4- | |
| | yl)imidazo[1,2-a]pyridine-6-carboxylic acid (Preparation 298) | |
| | R-NH₂: prep-HPLC-J | |
| 285 | 7-(Cyclopentyloxy)-N-(1-methyl-1H-pyrazol-3-yl)-2-(1-methyl-2- | 18.5 mg, 31.6% |
| | oxabicyclo[2.1.1]hexan-4-yl)imidazo[1,2-a]pyrimidine-6-carboxamide | LCMS m/z = 423.2 [M + H]⁺ |
| | | ¹H NMR (500 MHz, DMSO- |
| | 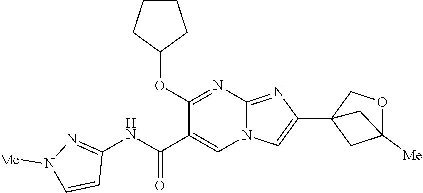 [SMILES: Cn1ccc(NC(=O)c2cn3cc(C45COC(C)(C4)C5)nc3nc2OC2CCCC2)n1] | d₆) δ: 1.35-1.49 (m, 3H), 1.64- 1.86 (m, 7H), 1.86-1.95 (m, 2H), 1.95-2.09 (m, 4H), 3.78 (s, 3H), 3.88 (s, 2H), 5.61 (tt, 1H), 6.58 (d, 1H), 7.56-7.70 (m, 1H), 9.29 (s, 1H), 10.23 (s, 1H). |
| | RCO₂H: 7-(cyclopentyloxy)-2-(1-methyl-2-oxabicyclo[2.1.1]hexan-4- | |
| | yl)imidazo[1,2-a]pyrimidine-6-carboxylic acid (Preparation 370) | |
| | R-NH₂: prep-HPLC-J | |
| 286 | 7-(Methoxymethyl)-N-(1-methyl-1H-pyrazol-3-yl)-2-(1-methyl-2- | 28.9 mg, 35.79% yield |
| | oxabicyclo[2.1.1]hexan-4-yl)imidazo[1,2-a]pyridine-6-carboxamide | LCMS m/z = 382.3 [M + H]⁺ |
| | | ¹H NMR (500 MHz, |
| | 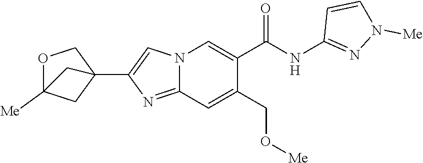 [SMILES: COCc1cc2nc(C34COC(C)(C3)C4)cn2cc1C(=O)Nc1ccn(C)n1] | DMSO-d₆) δ: 1.44 (s, 3H), 1.78 (dd, 2H), 2.03 (dd, 2H), 3.31 (s, 3H), 3.78 (s, 3H), 3.91 (s, 2H), 4.62 (s, 2H), 6.57 (d, 1H), 7.49-7.63 (m, 12H), 7.85 (s, 1H), 8.78 (s, 1H), 10.89 (s, 1H). |
| | RCO₂H: 7-(methoxymethyl)-2-(1-methyl-2-oxabicyclo[2.1.1]hexan-4- | |
| | yl)imidazo[1,2-a]pyridine-6-carboxylic acid (Preparation 316) | |
| | R-NH₂: 1-methylpyrazol-3-amine | |
| | prep-HPLC-J | |
| 287 | 7-(Methoxymethyl)-N-(1-methyl-2-oxo-1,2-dihydropyridin-3-yl)-2-(1- | 6.60 mg, 7.63% yield |
| | methyl-2-oxabicyclo[2.1.1]hexan-4-yl)imidazo[1,2-a]pyridine-6-carboxamide | LCMS m/z = 409.2 [M + H]⁺ |
| | | ¹H NMR (500 MHz, |
| | 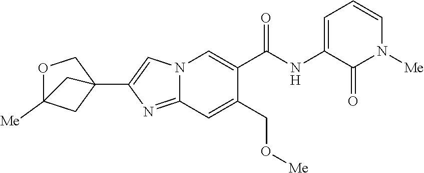 [SMILES: COCc1cc2nc(C34COC(C)(C3)C4)cn2cc1C(=O)Nc1cccn(C)c1=O] | DMSO-d₆) δ: 1.44 (s, 3H), 1.78-1.85 (m, 2H), 2.03-2.11 (m, 2H), 3.34 (s, 3H) 3.54 (s, 2H), 3.93 (s, 2H), 4.64 (s, 2H), 6.33 (t, 1H), 7.52 (dd, 1H), 7.61 (br s, 1H), 7.99 (br s, 1H), 8.31 (dd, 1H), 8.97 (s, 1H), 9.78 (s, 1 H). |
| | RCO₂H: 7-(methoxymethyl)-2-(1-methyl-2-oxabicyclo[2.1.1]hexan-4- | |
| | yl)imidazo[1,2-a]pyridine-6-carboxylic acid (Preparation 316) | |
| | R-NH₂: 3-amino-1-methylpyridin-2-one | |
| | prep-HPLC-J | |
| 288 | 7-(Methoxymethyl)-2-(1-methyl-2-oxabicyclo[2.1.1]hexan-4-yl)-N-(6- | 41.4 mg, 45.22 % yield. |
| | methylpyrazolo[1,5-a]pyrimidin-3-yl)imidazo[1,2-a]pyridine-6-carboxamide | LCMS m/z = 433.2 [M + H]⁺ |
| | | ¹H NMR (500 MHz, |
| | 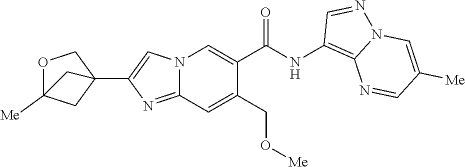 [SMILES: COCc1cc2nc(C34COC(C)(C3)C4)cn2cc1C(=O)Nc1cnn2cc(C)cnc12] | DMSO-d₆) δ ppm 1.44 (s, 3H), 1.79 (dd, 2H), 2.03 (dd, 2H), 2.32-2.39 (m, 3H), 3.35 (s, 3H), 3.92 (s, 2H), 4.67 (s, 2H), 7.52 (s, 1H), 7.91 (s, 1H), 8.42-8.58 (m, 1H), 8.89- 8.94 (m, 1H), 10.65 (s, 1H). |
| | RCO₂H: 7-(methoxymethyl)-2-(1-methyl-2-oxabicyclo[2.1.1]hexan-4- | |
| | yl)imidazo[1,2-a]pyridine-6-carboxylic acid (Preparation 316) | |
| | R-NH₂: 6-methylpyrazolo[1,5-a]pyrimidin-3-amine | |
| | prep-HPLC-J | |
| 289 | N-(6-(difluoromethyl)pyridin-2-yl)-2-(1-(fluoromethyl)-2-oxabicyclo[2.1.1] | 116.4 mg, 69.3% yield |
| | hexan-4-yl)-7-isopropoxyimidazo[1,2-a]pyridine-6-carboxamide | LCMS m/z = 461.2 [M + H]⁺ |
| | | ¹H NMR (500 MHz, |
| | 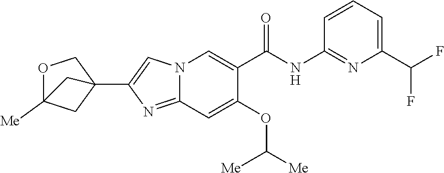 [SMILES: CC(C)Oc1cc2nc(C34COC(C)(C3)C4)cn2cc1C(=O)Nc1cccc(C(F)F)n1] | DMSO-d₆) δ: 1.42 (br d, 6H), 1.90-2.01 (m, 2H), 2.27 (br d, 2H), 4.02 (s, 2H), 4.67-4.80 (m, 2H), 5.01 (br s, 1H), 6.75-7.04 (m, 1H), 7.20-7.34 (m, 1H), 7.45-7.58 (m, 1H), 8.03-8.12 (m, 2H), 8.36 (br s, 1H), 9.16 (s, 1H), 11.07 (br s, 1H). |
| | RCO₂H: 2-(1-(fluoromethyl)-2-oxabicyclo[2.1.1]hexan-4-yl)-7- | |
| | isopropoxyimidazo[1,2-a]pyridine-6-carboxylic acid (Preparation 364) | |
| | R-NH₂: 6-(difluoromethyl)pyridin-2-amine hydrochloride. prep-HPLC-J | |
| 290 | N-(6-(difluoromethyl)pyridin-2-yl)-7-methoxy-8-methyl-2-(1-methyl-2- | 10 mg, 14.11% yield |
| | oxabicyclo[2.1.1]hexan-4-yl)imidazo[1,2-a]pyridine-6-carboxamide | LCMS m/z = 429.2 [M + H]⁺ |
| | | ¹H NMR (500 MHz, |
| | 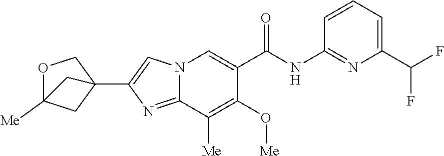 [SMILES: COc1c(C(=O)Nc2cccc(C(F)F)n2)cn2cc(C34COC(C)(C3)C4)nc2c1C] | DMSO-d₆) δ: 1.44 (s, 3H), 1.74-1.80 (m, 2H), 1.98-2.04 (m, 2H), 2.44 (s, 3H), 3.81- 3.86 (m, 1H), 3.83 (s, 2H), 3.88-3.92 (m, 2H), 6.78-7.05 (m, 1H), 7.46-7.53 (m, 1H), 7.85 (s, 1H), 8.09 (t, 1H), 8.39 (d, 1H), 8.88 (s, 1H), 10.97 (br s, 1H). |
| | RCO₂H: 7-methoxy-8-methyl-2-(1-methyl-2-oxabicyclo[2.1.1]hexan-4- | |
| | yl)imidazo[1,2-a]pyridine-6-carboxylic acid (Preparation 320) | |
| | R-NH₂: 6-(difluoromethyl)pyridin-2-amine hydrochloride. prep-HPLC-J | |
| 291 | 7-Methoxy-N-(2-methoxypyridin-3-yl)-8-methyl-2-(1-methyl-2- | 9.60 mg, 14.21% yield |
| | oxabicyclo[2.1.1]hexan-4-yl)imidazo[1,2-a]pyridine-6-carboxamide | LCMS m/z = 409.2 [M + H]⁺ |
| | | ¹H NMR (500 MHz, |
| | 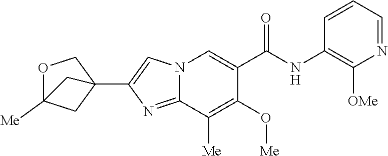 [SMILES: COc1ncccc1NC(=O)c1cn2cc(C34COC(C)(C3)C4)nc2c(C)c1OC] | DMSO-d₆) δ: 1.44 (s, 3H), 1.71-1.88 (m, 2H), 1.98-2.09 (m, 2H), 2.49 (s, 3H), 3.90 (s, 2H), 3.94 (s, 3H), 4.04 (s, 3H), 7.07 (dd, 1H), 7.93- 7.98 (m, 1H), 8.61-8.69 (m, 1H), 9.10 (s, 1H), 10.53 (s, 1H). |
| | RCO₂H: 7-methoxy-8-methyl-2-(1-methyl-2-oxabicyclo[2.1.1]hexan-4- | |
| | yl)imidazo[1,2-a]pyridine-6-carboxylic acid (Preparation 320) | |
| | R-NH₂: 2-methoxypyridin-3-amine hydrochloride. prep-HPLC-J | |
| 292 | 8-Fluoro-7-isopropoxy-2-(1-methyl-2-oxabicyclo[2.1.1]hexan-4-yl)-N-(6- | 41.1 mg, 24.74% yield |
| | methylpyrazolo[1,5-a]pyrimidin-3-yl)imidazo[1,2-a]pyridine-6-carboxamide | LCMS m/z = 465.3 [M + H]+ |
| | trifluoroacetate | 1H NMR (500 MHz, |
| | | DMSO-d₆) δ: 1.40-1.49 (m, |
| | 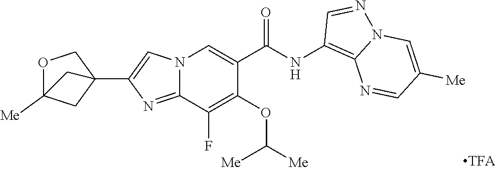 [SMILES: Cc1cnc2c(NC(=O)c3cn4cc(C56COC(C)(C5)C6)nc4c(F)c3OC(C)C)cnn2c1] | 9H), 1.75-1.85 (m, 2H), 2.04 (dd, 2H), 2.37 (s, 3H), 3.90 (m, 2H), 4.74 (spt, 1H), 8.01 (d, 1H), 8.49 (d, 1H), 8.64 (s, 1H), 8.95 (d, 1H), 9.00 (s, 1H), 10.45 (s, 1H). |
| | RCO₂H: 8-fluoro-7-isopropoxy-2-(1-methyl-2-oxabicyclo[2.1.1]hexan-4- | |
| | yl)imidazo[1,2-a]pyridine-6-carboxylic acid (Preparation 127) | |
| | R-NH₂: 6-methylpyrazolo[1,5-a]pyrimidin-3-amine. prep-HPLC-D | |
| 293 | N-(6-(difluoromethyl)pyridin-2-yl)-7-methoxy-2-(1-methyl-2- | 25.3 mg, 29.3% yield |
| | oxabicyclo[2.1.1]hexan-4-yl)imidazo[1,2-a]pyridine-6-carboxamide | LCMS m/z = 415.2 [M + H]⁺ |
| | | ¹H NMR (500 MHz, |
| | 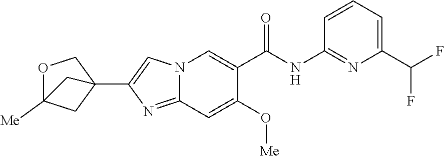 [SMILES: COc1cc2nc(C34COC(C)(C3)C4)cn2cc1C(=O)Nc1cccc(C(F)F)n1] | DMSO-d₆) δ: 1.43 (s, 3H), 1.76 (dd, 2H), 2.00 (dd, 2H), 3.89 (s, 2H), 3.96 (s, 3H), 6.79-7.05 (m, 1H), 7.10 (s, 1H), 7.48 (d, 1H), 7.76 (s, 1H), 8.08 (t, 1H), 8.38 (d, 1H), 8.96 (s, 1H), 10.78 (br s, 1H). |
| | RCO₂H: 7-methoxy-2-(1-methyl-2-oxabicyclo[2.1.1]hexan-4-yl)imidazo[1,2- | |
| | a]pyridine-6-carboxylic acid (Preparation 358) | |
| | R-NH₂: 6-(difluoromethyl)pyridin-2-amine hydrochloride. prep-HPLC-J | |
| 294 | N-(6-(difluoromethyl)pyridin-2-yl)-7-isopropoxy-2-(trifluoromethyl) | 17.0 mg, 14.78% yield |
| | imidazo[1,2-a]pyridine-6-carboxamide | LCMS m/z = 415.1 [M + H]⁺ |
| | | ¹H NMR (500 MHz, |
| | 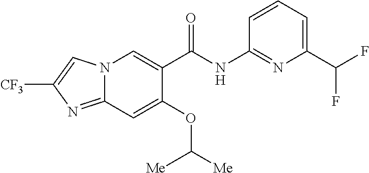 [SMILES: CC(C)Oc1cc2nc(C(F)(F)F)cn2cc1C(=O)Nc1cccc(C(F)F)n1] | DMSO-d₆) δ: 1.42 (br d, 6H), 4.96 (dq, 1H), 6.75- 7.06 (m, 1H), 7.27 (s, 1H), 7.49 (d, 1H), 8.09 (t, 1H), 8.36 (br d, 1H), 8.47 (s, 1H), 9.13 (s, 1H), 10.95 (s, 1H). |
| | RCO₂H: 7-isopropoxy-2-(trifluoromethyl)imidazo[1,2-a]pyridine-6-carboxylic | |
| | acid (Preparation 347) | |
| | R-NH₂: 6-(difluoromethyl)pyridin-2-amine hydrochloride. prep-HPLC-J | |
| 295 | N-(4-(difluoromethyl)pyrimidin-2-yl)-7-isopropoxy-2-(1-methyl-2- | 5.10 mg, 15.13% yield |
| | oxabicyclo[2.1.1]hexan-4-yl)imidazo[1,2-a]pyridine-6-carboxamide | LCMS m/z = 443.9 [M + H]⁺ |
| | trifluoroacetate | ¹H NMR (500 MHz, MeOH- |
| | | d₄) δ: 1.38 (br d, 6H), 1.52 |
| | 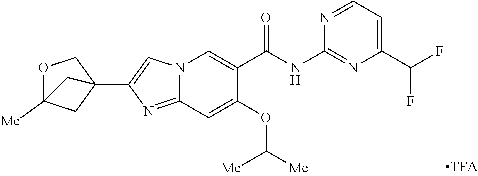 [SMILES: CC(C)Oc1cc2nc(C34COC(C)(C3)C4)cn2cc1C(=O)Nc1nccc(C(F)F)n1] | (s, 3H), 1.93-2.02 (m, 2H), 2.22 (dd, 2H), 4.05 (s, 2H), 4.93 (br dd, 1H), 6.59 (dd, 1H), 7.22 (s, 1H), 7.45 (d, 1H), 7.95 (s, 1H), 8.81 (d, 1H), 9.03 (s, 1H). |
| | RCO₂H: 7-isopropoxy-2-(1-methyl-2-oxabicyclo[2.1.1]hexan-4- | |
| | yl)imidazo[1,2-a]pyridine-6-carboxylic acid (Preparation 78) | |
| | R-NH₂: 4-(difluoromethyl)pyrimidin-2-amine hydrochloride. prep-HPLC-D | |
| 296 | N-(6-cyclopropylpyridin-2-yl)-7-isopropoxy-2-(1-methyl-2-oxabicyclo | 15.20 mg, 17.63% yield |
| | [2.1.1]hexan-4-yl)imidazo[1,2-a]pyridine-6-carboxamide trifluoroacetate | ¹H NMR (500 MHz, |
| | | DMSO-d₆) δ: 0.79-1.06 (m, |
| | 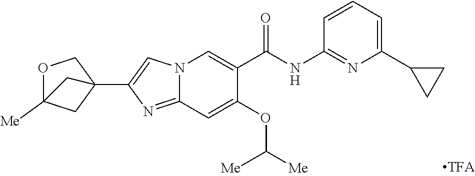 [SMILES: CC(C)Oc1cc2nc(C34COC(C)(C3)C4)cn2cc1C(=O)Nc1cccc(C2CC2)n1] | 4H) 1.39-1.58 (m, 9H) 1.84 (br d, 2H) 2.10 (br s, 3H) 3.93 (s, 2H) 5.07-5.11 (m, 1H) 7.12-7.43 (m, 2H) 7.61- 7.82 (m, 1H) 7.96 (br d, 1H) 9.26 (br s, 1H) 10.57 (br s, 1H). |
| | R-NH₂: 7-isopropoxy-2-(1-methyl-2-oxabicyclo[2.1.1]hexan-4- | |
| | yl)imidazo[1,2-a]pyridine-6-carboxylic acid (Preparation 78) | |
| | SM: (6-cyclopropyl)pyridin-2-amine | |
| | prep-HPLC-D | |
| 297 | 7-Isopropoxy-N-(1-methyl-1H-pyrazol-3-yl)-2-(1-methyl-2-oxabicyclo[2.1.1] | 18.10 mg, 22.52% yield |
| | hexan-4-yl)imidazo[1,2-a]pyridine-6-carboxamide trifluoroacetate | LCMS m/z = 396.0 [M + H]⁺ |
| | | ¹H NMR (500 MHz, MeOH- |
| | 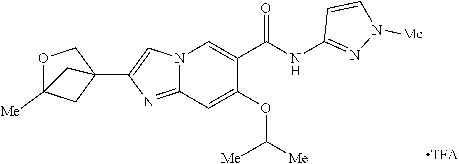 [SMILES: CC(C)Oc1cc2nc(C34COC(C)(C3)C4)cn2cc1C(=O)Nc1ccn(C)n1] | d₄) δ: 1.52 (s, 3H), 1.56 (d, 6H), 1.98 (dd, 2H), 2.17- 2.25 (m, 2H), 3.84 (s, 3H), 4.04 (s, 2H), 4.98-5.14 (m, 1H), 6.66 (d, 1H), 7.33 (s, 1H), 7.53 (d, 1H), 7.97 (s, 1H), 9.16 (s, 1H), |
| | RCO₂H: 7-isopropoxy-2-(1-methyl-2-oxabicyclo[2.1.1]hexan-4- | |
| | yl)imidazo[1,2-a]pyridine-6-carboxylic acid (Preparation 78) | |
| | R-NH₂: 1-methylpyrazol-3-amine hydrochloride. prep-HPLC-D | |
| 298 | 7-Isopropoxy-2-(1-methyl-2-oxabicyclo[2.1.1]hexan-4-yl)-N-(pyrazolo[1,5- | 13.2 mg, 15.34% yield |
| | a]pyridin-2-yl)imidazo[1,2-a]pyridine-6-carboxamide trifluoroacetate | LCMS m/z = 432.1 [M + H]⁺ |
| | | ¹H NMR (500 MHz, |
| | 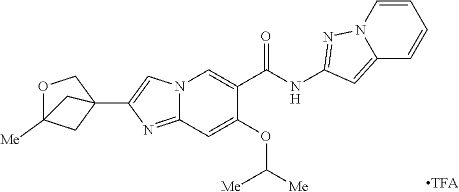 [SMILES: CC(C)Oc1cc2nc(C34COC(C)(C3)C4)cn2cc1C(=O)Nc1cc2ccccn2n1] | DMSO-d₆) δ: 1.44 (d, 9H), 1.85 (br d, 2H), 2.13 (br d, 2H), 3.94 (s, 2H), 4.91-5.06 (m, 1H), 6.78-7.34 (m, 4H), 7.66 (d, 1H), 7.87-8.16 (m, 1H), 8.59(d, 1H), 9.16 (s, 1H). |
| | RCO₂H: 7-isopropoxy-2-(1-methyl-2-oxabicyclo[2.1.1]hexan-4-yl)imidazo[1,2- | |
| | a]pyridine-6-carboxylic acid (Preparation 78) | |
| | R-NH₂: pyrazolo[1,5-a]pyridine-2-amine | |
| | prep-HPLC-D | |
| 299 | 7-Isopropoxy-2-(1-methyl-2-oxabicyclo[2.1.1]hexan-4-yl)-N-(pyrazolo[1,5- | 14.60 mg, 16.93% yield. |
| | a]pyrimidin-3-yl)imidazo[1,2-a]pyridine-6-carboxamide trifluoroacetate | LCMS m/z = 433.0 [M + H]⁺ |
| | | ¹H NMR (500 MHz, |
| | 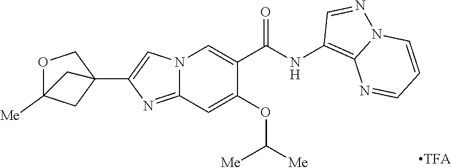 [SMILES: CC(C)Oc1cc2nc(C34COC(C)(C3)C4)cn2cc1C(=O)Nc1cnn2cccnc12] | DMSO-d₆) δ: 1.44 (s, 3H), 1.55 (d, 6H), 1.78 (dd, 2H), 1.95-2.04 (m, 2H), 3.90 (s, 2H), 4.82-5.11 (m, 1H), 7.07 (dd, 1H), 7.24 (s, 1H), 7.88 (s, 1H), 8.55 (s, 1H), 8.77 (s, 1H), 9.04-9.18 (m, 1H), 9.22 (s, 1H), 10.52 (s, 1H), |
| | RCO₂H: 7-isopropoxy-2-(1-methyl-2-oxabicyclo[2.1.1]hexan-4-yl)imidazo[1,2- | |
| | a]pyridine-6-carboxylic acid (Preparation 78) | |
| | R-NH₂: pyrazolo[1,5-a]pyrimidin-3-amine | |
| | prep-HPLC-D | |
| 300 | 7-Isopropoxy-N-(5-methoxypyrazolo[1,5-a]pyrimidin-3-yl)-2-(1-methyl-2- | 5.30 mg, 10.36% yield |
| | oxabicyclo[2.1.1]hexan-4-yl)imidazo[1,2-a]pyridine-6-carboxamide | LCMS m/z = 463.0 [M + H]⁺ |
| | | ¹H NMR (500 MHz, |
| | 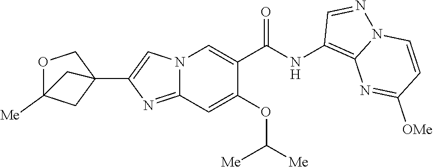 [SMILES: COc1ccn2ncc(NC(=O)c3cn4cc(C56COC(C)(C5)C6)nc4cc3OC(C)C)c2n1] | DMSO-d₆) δ: 1.44 (s, 3H), 1.55 (d, 6H), 1.82 (br d, 2H), 2.07 (br s, 2H), 3.92 (s, 2H), 4.00 (s, 3H), 4.96-5.24 (m, 1H), 6.59 (d, 1H), 7.28 (s, 1H), 7.86-8.10 (m, 1H), 8.64 (s, 1H), 8.77-8.97 (m, 1H), 9.23- 9.39 (m, 1H), 10.06 (s, 1H), |
| | RCO₂H: 7-isopropoxy-2-(1-methyl-2-oxabicyclo[2.1.1]hexan-4-yl)imidazo[1,2- | |
| | a]pyridine-6-carboxylic acid (Preparation 78) | |
| | R-NH₂: 5-methoxypyrazolo[l,5-a]pyrimidin-3-amine (Preparation X). | |
| | prep-HPLC-J | |
| 301 | 7-Isopropoxy-N-(6-methoxyimidazo[1,2-b]pyridazin-3-yl)-2-(1-methyl-2- | 31.4 mg, 61.36% yield |
| | oxabicyclo[2.1.1]hexan-4-yl)imidazo[1,2-a]pyridine-6-carboxamide | LCMS m/z = 463.0 [M + H]⁺ |
| | | ¹H NMR (500 MHz, |
| | 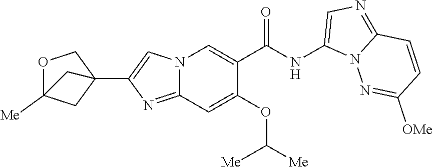 [SMILES: COc1ccc2ncc(NC(=O)c3cn4cc(C56COC(C)(C5)C6)nc4cc3OC(C)C)n2n1] | DMSO-d₆) δ: 1.43 (s, 3H), 1.49 (d, 6H), 1.78 (br d, 2H), 2.02 (br d, 2H), 3.90 (s, 2H), 4.05 (s, 3H), 4.89-5.06 (m, 1H), 6.89 (d, 1H), 7.24 (s, 1H), 7.95 (s, 1H), 8.04 (d, 1H), 9.21 (s, 1H), 10.37 (s, 1H). |
| | RCO₂H: 7-isopropoxy-2-(1-methyl-2-oxabicyclo[2.1.1]hexan-4-yl)imidazo[1,2- | |
| | a]pyridine-6-carboxylic acid (Preparation 78) | |
| | R-NH₂: 6-methoxyimidazo[1,2-b]pyridazin-3-amine (Preparation X). | |
| | prep-HPLC-J | |
| 302 | 8-Fluoro-7-isopropoxy-2-(1-methyl-2-oxabicyclo[2.1.1]hexan-4-yl)-N- | 16.3 mg, 22.97% yield |
| | (pyrazolo[1,5-a]pyrimidin-3-yl)imidazo[1,2-a]pyridine-6-carboxamide | LCMS m/z = 451.0 [M + H]⁺ |
| | | ¹H NMR (400 MHz, MeOH- |
| | 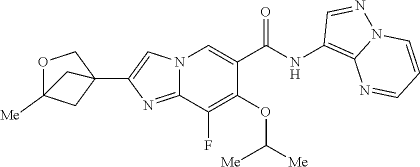 [SMILES: CC(C)Oc1c(C(=O)Nc2cnn3cccnc23)cn2cc(C34COC(C)(C3)C4)nc2c1F] | d₄) δ: 1.53 (s, 3H), 1.58 (dd, 6H), 1.94 (d, 1H), 2.15-2.24 (m, 2H), 4.06 (s, 2H), 7.05 (dd, 1H), 7.91 (d, 1H), 8.55 (dd, 1H), 8.80 (s, 1H), 8.89 (dd, 1H), 9.03 (d, 1H), |
| | RCO₂H: 8-fluoro-7-isopropoxy-2-(1-methyl-2-oxabicyclo[2.1.1]hexan-4- | |
| | yl)imidazo[1,2-a]pyridine-6-carboxylic acid (Preparation 127) | |
| | R-NH₂: pyrazolo[1,5-a]pyrimidin-3-amine | |
| | SiO2; (3:1 EtOAc/EtOH)/Heptane | |
| 303 | N-(6-(difluoromethyl)pyridin-2-yl)-8-fluoro-7-isopropoxy-2-(tetrahydro-2H- | 13.6 mg, 16.29% yield |
| | pyran-4-yl)imidazo[1,2-a]pyridine-6-carboxamide | ¹H NMR (400 MHz, CDCl₃) |
| | | δ: 1.47 (dd, 6H), 1.68-1.84 |
| | 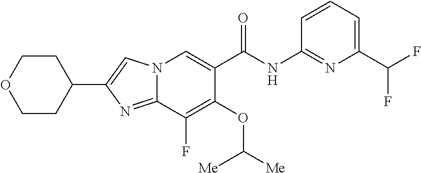 [SMILES: CC(C)Oc1c(C(=O)Nc2cccc(C(F)F)n2)cn2cc(C3CCOCC3)nc2c1F] | (m, 2H), 1.95-2.05 (m, 2H), 2.84-3.13 (m, IH), 3.50 (td, 2H), 3.90-4.10 (m, 2H), 4.95 (td, 1H), 6.24-6.71 (m, 1H), 7.28-7.43 (m, 2H), 7.83 (t, 1H), 8.37 (dd, 1H), 8.80 (d, 1H), 10.68 (s, 1H) |
| | RCO₂H: 8-fluoro-7-isopropoxy-2-(tetrahydro-2H-pyran-4-yl)imidazo[1,2- | |
| | a]pyridine-6-carboxylic acid (Preparation 342) | |
| | R-NH₂: 6-(difluoromethyl)pyridin-2-amine | |
| | SiO2; (3:1 EtOAc/EtOH)/Heptane | |
| 304 | 7-Isopropoxy-N-(1-methyl-1H-pyrazol-3-yl)-2-(1-methyl-2-oxabicyclo[2.1.1] | 19.60 mg, 31.28% yield |
| | hexan-4-yl)imidazo[1,2-a]pyrimidine-6-carboxamide | LCMS m/z = 397.0 [M + H]⁺ |
| | | ¹H NMR (400 MHz, CDCl₃) |
| | 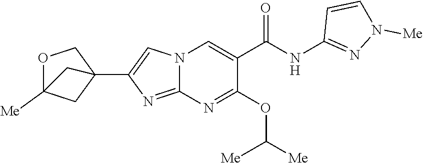 [SMILES: CC(C)Oc1nc2nc(C34COC(C)(C3)C4)cn2cc1C(=O)Nc1ccn(C)n1] | δ: 1.53 (s, 3H), 1.58 (d, 6H), 1.95 (dd, 2H), 2.03- 2.19 (m, 2H), 3.86 (s, 3H), 4.07 (s, 2H), 5.76-5.80 (m, 1H), 6.75 (d, 1H), 7.19-7.40 (m, 2H), 9.21 (s, 1H), 10.09 (s, 1H) |
| | RCO₂H: 7-isopropoxy-2-(1-methyl-2-oxabicyclo[2.1.1]hexan-4- | |
| | yl)imidazo[1,2-a]pyrimidine-6-carboxylic acid (Preparation 128) | |
| | R-NH₂: 1-methylpyrazol-3-amine | |
| | SiO2; (3:1 EtOAc/EtOH)/Heptane | |
| 305 | 7-Isopropoxy-N-(2-methoxypyridin-3-yl)-2-(1-methyl-2-oxabicyclo[2.1.1] | 9.0 mg, 10.21% yield |
| | hexan-4-yl)imidazo[1,2-a]pyrimidine-6-carboxamide trifluoroacetate | ¹H NMR (500 MHz, |
| | | DMSO-d₆) δ: 1.44 (s, 3H), |
| | 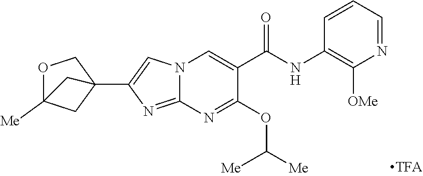 [SMILES: COc1ncccc1NC(=O)c1cn2cc(C34COC(C)(C3)C4)nc2nc1OC(C)C] | 1.55 (d, 6H), 1.79 (dd, 2H), 2.05 (br d, 2H), 3.90 (s, 2H) 4.04 (s, 3H), 5.58-5.68 (m, 1H), 7.09 (s, 1H), 7.81 (s, 1H), 7.96 (dd, 1H), 8.72 (dd, 1H), 9.56 (s, 1H), 10.14 (s, 1H) |
| | RCO₂H: 7-isopropoxy-2-(1-methyl-2-oxabicyclo[2.1.1]hexan-4- | |
| | yl)imidazo[1,2-a]pyrimidine-6-carboxylic acid (Preparation 128) | |
| | R-NH₂: 2-methoxypyridin-3-amine | |
| | prep-HPLC-D | |
| 306 | N-(5-fluoro-2-methoxypyridin-3-yl)-7- | 23.3 mg, 16.81% |
| | isopropoxy-2-(1-methyl-2-oxabicyclo[2.1.1]hexan-4-yl)imidazo[1,2- | LCMS m/z = 442.9 [M + H]⁺ |
| | a]pyrimidine-6-carboxamide trifluoroacetate | ¹H NMR (400 MHz, MeOH- |
| | | d₄) δ: 1.54 (s, 3H), 1.68 (d, |
| | 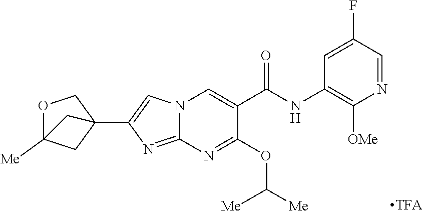 [SMILES: COc1ncc(F)cc1NC(=O)c1cn2cc(C34COC(C)(C3)C4)nc2nc1OC(C)C] | 6H), 1.92-2.05 (m, 2H), 2.20 (s, 2H), 4.05 (s, 2H), 4.12 (s, 3H), 5.74-5.90 (m, 1H), 7.79-7.97 (m, 2H), 8.71 (d, 1H), 9.62 (s, 1H) |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128) | |
| | R-NH₂: 5-fluoro-2-methoxypyridin-3-amine. prep-HPLC-D | |
| 307 | 7-Isopropoxy-2-(1-methyl-2-oxabicyclo[2.1.1]hexan-4-yl)-N-(pyrazolo[1,5- | 19.30 mg, 28.26% yield |
| | a]pyrimidin-3-yl)imidazo[1,2-a]pyrimidine-6-carboxamide | LCMS m/z = 434.0 [M + H]⁺ |
| | | ¹H NMR (400 MHz, MeOH- |
| | 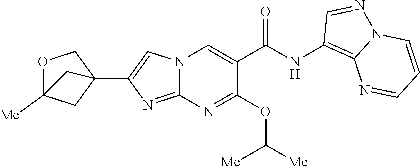 [SMILES: CC(C)Oc1nc2nc(C34COC(C)(C3)C4)cn2cc1C(=O)Nc1cnn2cccnc12] | d₄) δ: 1.52 (s, 3H), 1.68 (d, 6H), 1.87-1.94 (m, 2H), 2.11-2.19 (m, 2H), 4.03 (s, 2H), 5.76 (quin, 1H), 7.04 (dd, 1H), 7.66 (s, 1H), 8.55 (dd, 1H), 8.80 (s, 1H), 8.88 (dd, 1H), 9.42 (s, 1H) |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | R-NH2: pyrazolo[1,5-a]pyrimidin-3-amine. SiO2; (3:1 EtOAc/EtOH)/Heptane | |
| 308 | 7-Isopropoxy-N-(6-methoxyimidazo[1,2-b]pyridazin-3-yl)-2-(1-methyl-2- | 11.90 mg, 13.1% yield |
| | oxabicyclo[2.1.1]hexan-4-yl)imidazo[1,2-a]pyrimidine-6-carboxamide | LCMS m/z = 464.0 [M + H]⁺ |
| | trifluoroacetate | ¹H NMR (500 MHz, |
| | | DMSO-d₆) δ: 1.43 (s, 3H), |
| | 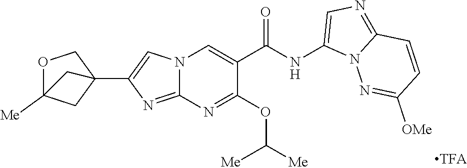 [SMILES: COc1ccc2ncc(NC(=O)c3cn4cc(C56COC(C)(C5)C6)nc4nc3OC(C)C)n2n1] | 1.56 (d, 6H), 1.77 (dd, 2H), 2.01 (dd, 2H), 3.88 (s, 2H), 4.02 (s, 3H), 5.50-5.67 (m, 1H), 6.59 (d, 1H), 7.74 (s, 1H), 8.64 (s, 1H), 8.89 (d, 1H), 9.49 (s, 1H), 9.91 (s, 1H). |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | R-NH₂: 6-methoxyimidazo[1,2-b]pyridazin-3-amine (Preparation X) | |
| | prep-HPLC-D | |
| 309 | N-(5-(difluoromethyl)pyrazolo[1,5-a]pyrimidin-3-yl)-7-isopropoxy-2-(1- | 8.80 mg, 9.33% yield |
| | methyl-2 oxabicyclo[2.1.1]hexan-4-yl)imidazo[1,2-a]pyrimidine-6-carboxamide | LCMS m/z = 483.9 [M + H]⁺ |
| | trifluoroacetate | ¹H NMR (500 MHz, |
| | | DMSO-d₆) δ: 1.60 (s, 3H), |
| | 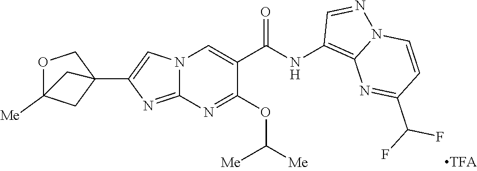 [SMILES: CC(C)Oc1nc2nc(C34COC(C)(C3)C4)cn2cc1C(=O)Nc1cnn2ccc(C(F)F)nc12] | 1.75 (s, 6H), 2.05 (s, 2H), 2.30 (s, 2H), 4.10 (s, 2H), 5.90 (t, 1H), 7.40 (t, 1H), 8.10-8.50 (br s), 8.60 (s, 1H), 9.00 (s, 1H), 9.40 (s, 1H), 10.35 (s, 1H). |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | R-NH₂: 5-(difluoromethyl)pyrazolo[1,5-a]pyrimidin-3-amine (Preparation X) | |
| | prep-HPLC-D | |
| 310 | N-(6-fluoropyrazolo[1,5-a]pyrimidin-3-yl)-7-isopropoxy-2-(1,3,3-trimethyl-2- | 4.10 mg, 5.91% yield |
| | oxabicyclo[2.1.1]hexan-4-yl)imidazo[1,2-a]pyrimidine-6-carboxamide | LCMS m/z = 480.2 [M + H]⁺ |
| | | ¹H NMR (500 MHz, |
| | 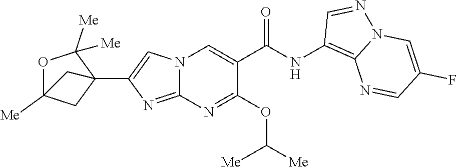 [SMILES: CC(C)Oc1nc2nc(C34CC(C)(C3)OC4(C)C)cn2cc1C(=O)Nc1cnn2cc(F)cnc12] | DMSO-d₆) δ: 1.22-1.40 (m, 9H), 1.55 (d, 6H), 1.87 (br d, 2H), 2.18 (dd, 2H), 5.57 (spt, 1H), 7.73 (s, 1H), 8.76 (s, 1H), 8.82 (d, 1H), 9.44 (s, 1H), 9.54 (dd, 1H), 10.37 (s, 1H). |
| | RCO₂H: 7-isopropoxy-2-(1,3,3-trimethyl-2-oxabicyclo[2.1.1]hexan-4-yl)imidazo | |
| | [1,2-a]pyrimidine-6-carboxylic acid (Preparation 332). | |
| | R-NH₂: 6-fluoropyrazolo[1,5-a]pyrimidin-3-amine hydrochloride | |
| | (Preparation X). prep-HPLC-D | |
| 311 | 7-Isopropoxy-N-(6-methylpyrazolo[1,5-a]pyrimidin-3-yl)-2-(1,3,3-trimethyl-2- | 0.9 mg, 1.06% yield |
| | oxabicyclo[2.1.1]hexan-4-yl)imidazo[1,2-a]pyrimidine-6-carboxamide | ¹H NMR (500 MHz, |
| | trifluoroacetate | DMSO-d₆) δ: 1.26-1.37 (m, |
| | | 9H), 1.55 (d, 6H), 1.88 (br d, |
| | 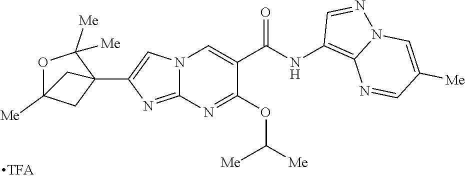 [SMILES: Cc1cnc2c(NC(=O)c3cn4cc(C56CC(C)(C5)OC6(C)C)nc4nc3OC(C)C)cnn2c1] | 2H), 2.18 (dd, 2H), 2.34 (s, 3H), 5.57 (quin, 1H), 7.74 (s, 1H), 8.49 (d, 1H), 8.66 (s, 1H), 8.89-9.01 (m, 1H), 9.44 (s, 1H), 10.33 (s, 1H). |
| | RCO₂H: 7-isopropoxy-2-(1,3,3-trimethyl-2-oxabicyclo[2.1.1]hexan-4- | |
| | yl)imidazo[1,2-a]pyrimidine-6-carboxylic acid (Preparation 332). | |
| | RNH₂: 6-methylpyrazolo[1,5-a]pyrimidin-3-amine. prep-HPLC-D | |
| 312 | 7-Cyclopropoxy-N-(6-(difluoromethyl)pyridin-2-yl)-2-(1-methyl-2-oxabicyclo | 6.90 mg, 15.7% yield |
| | [2.1.1]hexan-4-yl)imidazo[1,2-a]pyrimidine-6-carboxamide trifluoroacetate | LCMS m/z = 442.0 [M + H]⁺ |
| | | ¹H NMR (500 MHz, MeOH- |
| | 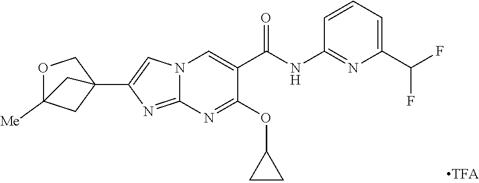 [SMILES: CC12CC(c3cn4cc(C(=O)Nc5cccc(C(F)F)n5)c(OC5CC5)nc4n3)(CO1)C2] | d₄) δ: 0.99-1.14 (m, 4H), 1.54 (s, 3H), 2.01 (dd, 2H), 2.23 (dd, 2H), 4.07 (s, 2H), 4.72 (br s, 1H), 6.51-6.83 (m, 1H), 7.43-7.55 (m, 1H), 7.51 (d, 1H), 7.94 (s, 1H), 8.05 (t, 1H), 8.42 (br s, 1H), 9.48 (s, 1H). |
| | R-NH₂: 7-cyclopropoxy-2-(1-methyl-2-oxabicyclo[2.1.1]hexan-4-yl)imidazo[1,2- | |
| | a]pyrimidine-6-carboxylic acid (Preparation 333). | |
| | R-NH2: 6-(difluoromethyl)pyridin-2-amine. prep-HPLC-D | |
| 313 | N-(1-(cyanomethyl)-1H-pyrazol-3-yl)-7-isopropoxy-2-(1-methyl-2- | 22.70 mg, 42.7% yield |
| | oxabicyclo[2.1.1]hexan-4-yl)imidazo[1,2-a]pyridine-6-carboxamide | LCMS m/z = 421.0 [M + H]⁺ |
| | | ¹H NMR (500 MHz, |
| | 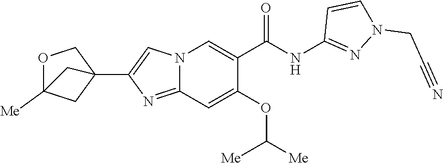 [SMILES: CC(C)Oc1cc2nc(C34COC(C)(C3)C4)cn2cc1C(=O)Nc1ccn(CC#N)n1] | DMSO-d₆) δ: 1.40-1.44 (m, 9H), 1.76 (dd, 2H), 1.99 (br d, 2H), 3.88 (s, 2H), 4.88 (spt, 1H), 5.44 (s, 2H), 6.72 (d, 1H), 7.11 (s, 1H), 7.72- 7.85 (m, 2H), 8.98 (s, 1H), 10.57 (br s, 1H). |
| | RCO₂H: 7-isopropoxy-2-(1-methyl-2-oxabicyclo[2.1.1]hexan-4- | |
| | yl)imidazo[1,2-a]pyridine-6-carboxylic acid (Preparation 78) | |
| | R-NH₂: 2-(3-aminopyrazol-1-yl)acetonitrile | |
| | prep-HPLC-F | |
| 314 | 7-Isopropoxy-N-(1-(2-methoxy ethyl)-1H-pyrazol-3-yl)-2-(1-methyl-2- | 24.4 mg, 42.55% yield |
| | oxabicyclo[2.1.1]hexan-4-yl)imidazo[1,2-a]pyridine-6-carboxamide | LCMS m/z = 454.3 [M + H]⁺ |
| | | ¹H NMR (500 MHz, |
| | 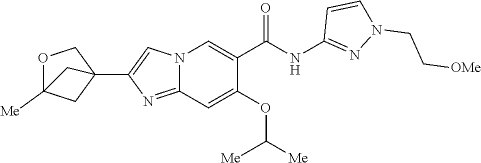 [SMILES: COCCn1ccc(NC(=O)c2cn3cc(C45COC(C)(C4)C5)nc3cc2OC(C)C)n1] | DMSO-d₆) δ: 1.41 (d, 6H), 1.44 (s, 3H), 1.78 (br s, 2H), 1.95-2.03 (m, 2H), 3.28-3.31 (m, 5H), 3.90 (s, 2H), 4.08 (t, 2H), 4.92 (br s, 1H), 6.59 (d, 1H), 7.14 (s, 1H), 7.67 (d, 1H), 7.84 (br s, 1H), 9.02 (s, 1H), 10.51 (br s, 1H). |
| | RCO₂H: 7-isopropoxy-2-(1-methyl-2-oxabicyclo[2.1.1]hexan-4-yl)imidazo[1,2- | |
| | a]pyridine-6-carboxylic acid (Preparation 78) | |
| | RNH₂: 1-(3-methoxypropyl)pyrazol-3-amine | |
| | prep-HPLC-F | |
| 315 | N-(1-cyclopentyl-1H-pyrazol-3-yl)-7-isopropoxy-2-(1-methyl-2-oxabicyclo | 15.5 mg, 21.75% yield |
| | [2.1.1]hexan-4-yl)imidazo[1,2-a]pyridine-6-carboxamide trifluoroacetate | LCMS m/z = 450.3 [M + H]⁺ |
| | | ¹H NMR (500 MHz, |
| | 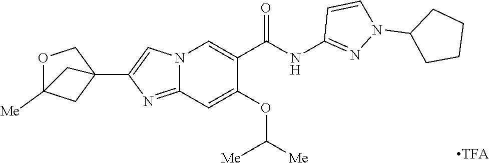 [SMILES: CC(C)Oc1cc2nc(C34COC(C)(C3)C4)cn2cc1C(=O)Nc1ccn(C2CCCC2)n1] | DMSO-d₆) δ: 1.41 (d, 6H), 1.45 (s, 3H), 1.58-1.68 (m, 2H), 1.73-1.80 (m, 2H), 1.83-1.87 (m, 2H), 1.87-1.94 (m, 2H), 2.01-2.09 (m, 2H), 2.12 (br d, 2H), 3.93 (s, 2H), 4.63 (quin, 1H), 4.98 (dt, 1H), 6.58 (d, 1H), 7.20 (s, 1H), 7.73 (s, 1H), 7.98 (br s, 1H), 9.05 (s, 1H), 10.74 (br s, 1H). |
| | RCO₂H: 7-isopropoxy-2-(1-methyl-2-oxabicyclo[2.1.1]hexan-4-yl)imidazo[1,2- | |
| | a]pyridine-6-carboxylic acid (Preparation 78) | |
| | R-NH₂: 1-cyclopentyl-1H-pyrazol-3-amine | |
| | prep-HPLC-D | |
| 316 | 7-Isopropoxy-N-(2-methyl-2H-1,2,3-triazol-4-yl)-2-(1-methyl-2-oxabicyclo | 20.9 mg, 32.38% yield |
| | [2.1.1]hexan-4-yl)imidazo[1,2-a]pyridine-6-carboxamide trifluoroacetate | LCMS m/z = 397.3 [M + H]⁺ |
| | | ¹H NMR (500 MHz, |
| | 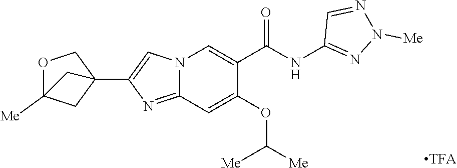 [SMILES: CC(C)Oc1cc2nc(C34COC(C)(C3)C4)cn2cc1C(=O)Nc1cnn(C)n1] | DMSO-d₆) δ: 1.40 (d, 6H), 1.45 (s, 3H), 1.81-1.87 (m, 2H), 2.12 (br d, 2H), 3.93 (s, 2H), 4.11 (s, 3H), 4.97 (spt, 1H), 7.17-7.25 (m, 1H), 7.98 (s, 1H), 9.09 (s, 1H), 11.04 (br s, 1H). |
| | RCO₂H: 7-isopropoxy-2-(1-methyl-2-oxabicyclo[2.1.1]hexan-4-yl)imidazo[1,2- | |
| | a]pyridine-6-carboxylic acid (Preparation 78) | |
| | R-NH₂: 2-methyltriazol-4-amine | |
| | prep-HPLC-D | |
| 317 | 7-Isopropoxy-N-(isoxazol-5-yl)-2-(1-methyl-2-oxabicyclo[2.1.1]hexan-4-yl) | 11.5 mg, 18.3% yield |
| | imidazo[1,2-a]pyridine-6-carboxamide trifluoroacetate | LCMS m/z = 383.2 [M + H]⁺ |
| | | ¹H NMR (500 MHz, |
| | 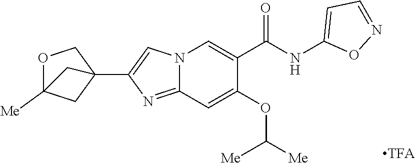 [SMILES: CC(C)Oc1cc2nc(C34COC(C)(C3)C4)cn2cc1C(=O)Nc1ccno1] | DMSO-d₆) δ: 1.37 (d, 6H), 1.43 (s, 3H), 1.72-1.80 (m, 2H), 2.00 (dd, 2H), 3.88 (s, 2H), 4.81 (spt, 1H), 6.39 (br s, 1H), 7.03-7.13 (m, 1H), 7.73 (s, 1H), 8.53 (d, 1H), 8.94 (s, 1H), 11.57 (br s, 1H). |
| | RCO₂H: 7-isopropoxy-2-(1-methyl-2-oxabicyclo[2.1.1]hexan-4-yl)imidazo[1,2- | |
| | a]pyridine-6-carboxylic acid (Preparation 78) | |
| | R-NH₂: isoxazole-5-amine. prep-HPLC-D | |
| 318 | 7-Isopropoxy-N-(2-methyl-2H-indazol-7-yl)-2-(1-methyl-2-oxabicyclo[2.1.1] | 40.8 mg, 72.4% yield LCMS |
| | hexan-4-yl)imidazo[1,2-a]pyridine-6-carboxamide | m/z = 446.2 [M + H]+ ¹H |
| | | NMR (500 MHz, DMSO-d₆) |
| | 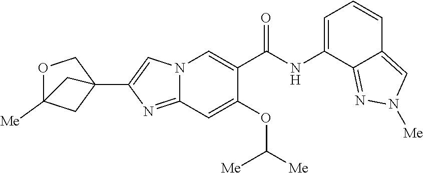 [SMILES: CC(C)Oc1cc2nc(C34COC(C)(C3)C4)cn2cc1C(=O)Nc1cccc2cn(C)nc12] | δ: 1.44 (s, 3H), 1.64 (d, 6H), 1.79 (br s, 2H), 2.02 (br s, 2H), 3.90 (s, 2H), 4.22 (s, 3H), 5.13 (br s, 1H), 7.01- 7.11 (m, 1H), 7.28 (br s, 1H), 7.44 (d, 1H), 7.89 (br s, 1H), 8.25 (d, 1H), 8.41 (s, 1H), 9.28 (br s, 1H), 10.80 (br s, 1H). |
| | RCO₂H: 7-isopropoxy-2-(1-methyl-2-oxabicyclo[2.1.1]hexan-4-yl)imidazo[1,2- | |
| | a]pyridine-6-carboxylic acid (Preparation 78) | |
| | R-NH₂: 2-methylimidazol-7-amine | |
| | prep-HPLC-F | |
| 319 | 7-Isopropoxy-2-(1-methyl-2-oxabicyclo[2.1.1]hexan-4-yl)-N-(1,5- | 10.8 mg, 19.26% yield |
| | naphthyridin-4-yl)imidazo[1,2-a]pyridine-6-carboxamide | LCMS m/z = 444.2 [M + H]⁺ |
| | | ¹H NMR (500 MHz, |
| | 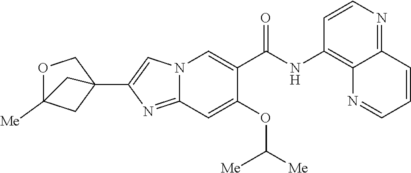 [SMILES: CC(C)Oc1cc2nc(C34COC(C)(C3)C4)cn2cc1C(=O)Nc1ccnc2cccnc12] | DMSO-d6) δ: 1.44 (s, 3H), 1.62 (d, 6H), 1.79 (br d, 2H), 2.03 (br s, 2H), 3.91 (s, 2H), 5.09-5.27 (m, 1H), 7.31 (s, 1H), 7.93-7.96 (m, 1H), 8.50 (dd, 1H), 8.74 (d, 1H), 8.98 (d, 1H), 9.02-9.05 (m, 1H), 9.36 (br s, 1H), 12.01 (s, 1H). |
| | RCO₂H: 7-isopropoxy-2-(1-methyl-2-oxabicyclo[2.1.1]hexan-4-yl)imidazo[1,2- | |
| | a]pyridine-6-carboxylic acid (Preparation 78) | |
| | R-NH₂: 1,5-naphthyridin-4-amine | |
| | prep-HPLC-F | |
| 320 | 7-Isopropoxy-2-(1-methyl-2-oxabicyclo[2.1.1]hexan-4-yl)-N-(1,6- | 25.3 mg, 45.12% yield |
| | naphthyridin-8-yl)imidazo[1,2-a]pyridine-6-carboxamide | LCMS m/z = 444.2 [M + H]⁺ |
| | | ¹H NMR (500 MHz, |
| | 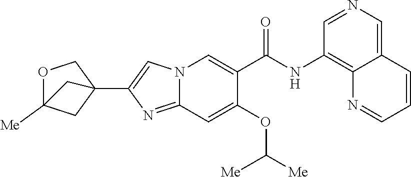 [SMILES: CC(C)Oc1cc2nc(C34COC(C)(C3)C4)cn2cc1C(=O)Nc1cncc2cccnc12] | DMSO-d₆) δ: 1.45 (s, 3H), 1.62 (d, 6H), 1.80 (br d, 2H), 2.05 (br d, 2H), 3.91 (s, 2H), 5.18 (br s, 1H), 7.32 (s, 1H), 7.88 (dd, 1H), 7.94 (br s, 1H), 8.70 (dd, 1H), 9.14-9.27 (m, 2H), 9.38 (br s, 1H), 9.93 (s, 1H), 11.68 (s, 1H) |
| | RCO₂H: 7-isopropoxy-2-(1-methyl-2-oxabicyclo[2.1.1]hexan-4-yl)imidazo[1,2- | |
| | a]pyridine-6-carboxylic acid (Preparation 78) | |
| | R-NH₂: 1,6-naphthyridin-8-amine | |
| | prep-HPLC-F | |
| 321 | N-(imidazo[1,2-b]pyridazin-3-yl)-7-isopropoxy-2-(1-methyl-2-oxabicyclo[2.1.1] | 47.40 mg, 27.44% yield |
| | hexan-4-yl)imidazo[1,2-a]pyridine-6-carboxamide 2,2,2-trifluoroacetate | LCMS m/z = 433.2 [M + H]⁺ |
| | | ¹H NMR (500 MHz, |
| | 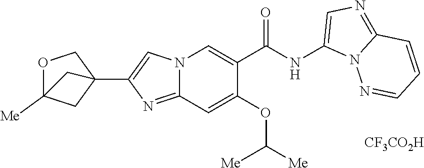 [SMILES: CC(C)Oc1cc2nc(C34COC(C)(C3)C4)cn2cc1C(=O)Nc1cnc2cccnn12 ; "CF3CO2H"] | DMSO-d6) δ: 1.46 (s, 3H), 1.58 (d, 6H), 1.83-1.90 (m, 2H), 2.13 (br s, 2H), 3.95 (s, 2H), 5.18-5.26 (m, 1H), 7.24-7.33 (m, 1H), 7.36 (s, 2H), 8.08-8.12 (m, 1H), 8.21 (dd, 1H), 8.66 (dd, 1H), 9.41 (s, 1H), 10.98 (s, 1H) |
| | RCO₂H: 7-isopropoxy-2-(1-methyl-2-oxabicyclo[2.1.1]hexan-4-yl)imidazo[1,2- | |
| | a]pyridine-6-carboxylic acid (Preparation 78) | |
| | RNH₂: imidazo[1,2-b]pyridazine-3-amine | |
| | Prep-HPLC-D | |
| 322 | N-(6-cyclopropylpyrazolo[1,5-a]pyrimidin-3-yl)-7-isopropoxy-2-(1-methyl- | 15.2 mg, 25.44% yield |
| | 2-oxabicyclo[2.1.1]hexan-4-yl)imidazo[1,2-a]pyridine-6-carboxamide | LCMS m/z = 473.2 [M + H]⁺ |
| | | ¹H NMR (500 MHz, |
| | 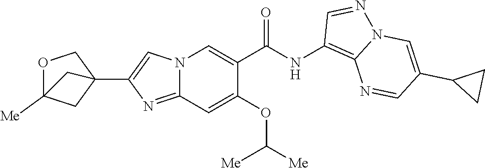 [SMILES: CC(C)Oc1cc2nc(C34COC(C)(C3)C4)cn2cc1C(=O)Nc1cnn2cc(C3CC3)cnc12] | DMSO-d₆) δ: 0.87-0.95 (m, 2H), 0.97-1.05 (m, 2H), 1.44 (s, 3H), 1.54 (d, 6H), 1.80 (br s, 2H), 2.00-2.10 (m, 3H), 3.91 (s, 2H), 5.01-5.15 (m, 1H), 7.25 (s, 1H), 7.93 (br s, 1H), 8.43-8.93 (m, 2H), 9.24 (br s, 1H), 10.49 (s, 1H). |
| | RCO₂H: 7-isopropoxy-2-(1-methyl-2-oxabicyclo[2.1.1]hexan-4-yl)imidazo[1,2- | |
| | a]pyridine-6-carboxylic acid (Preparation 78) | |
| | R-NH₂: 6-cyclopropylpyrazolo[1,5-a]pyrimidin-3-amine. prep-HPLC-F | |
| 323 | N-(6-(difluoromethyl)pyridin-2-yl)-2-(1-methyl-2-oxabicyclo[2.1.1]hexan-4-yl)- | 60 mg, 44.8% yield |
| | 7-((1,1,1-trifluoropropan-2-yl)oxy)imidazo[1,2-a]pyridine-6-carboxamide | LCMS m/z = 497.1 [M + H]⁺ |
| | | ¹H NMR (400 MHz, CDCl₃) |
| | 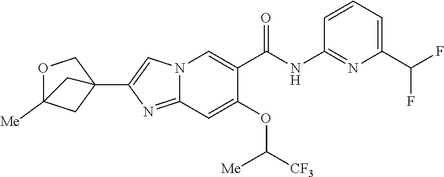 [SMILES: CC(Oc1cc2nc(C34COC(C)(C3)C4)cn2cc1C(=O)Nc1cccc(C(F)F)n1)C(F)(F)F] | δ: 1.56 (s, 3H), 1.77 (d, 3H), 2.00 (dd, 2H), 2.12 (br d, 2H), 4.09 (s, 2H), 4.90-5.09 (m, 1H), 6.32-6.72 (m, 1H), 7.21 (br s, 1H), 7.41-7.54 (m, 2H), 7.92 (t, 1H), 8.43- 8.51 (m, 1H), 9.09 (s, 1H), 9.43 (br s, 1H), 10.05 (s, 1H). |
| | RCO₂H: 2-(1-methyl-2-oxabicyclo[2.1.1]hexan-4-yl)-7-((1,1,1-trifluoropropan- | |
| | 2-yl)oxy)imidazo[1,2-a]pyridine-6-carboxylic acid (Preparation 318) | |
| | R-NH₂: 6-(difluoromethyl)pyridin-2-amine hydrochloride. prep-HPLC-F | |
| 324 | N-(6-(difluoromethyl)pyridin-2-yl)-3-fluoro-7-isopropoxy-2-(1-methyl-2- | 6.10 mg, 11.83% yield |
| | oxabicyclo[2.1.1]hexan-4-yl)imidazo[1,2-a]pyridine-6-carboxamide | LCMS m/z = 461.2 [M + H]⁺ |
| | trifluoroacetate | ¹H NMR (400 MHz, CDCl₃) |
| | | δ: 1.57 (s, 3H), 1.65 (d, 6H), |
| | 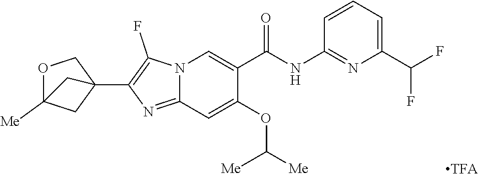 [SMILES: CC(C)Oc1cc2nc(C34COC(C)(C3)C4)c(F)n2cc1C(=O)Nc1cccc(C(F)F)n1] | 2.05 (dd, 2H), 2.25-2.34 (m, 2H), 4.17 (s, 2H), 5.04-5.15 (m, 1H), 6.36-6.68 (m, 1H), 7.48 (d, 1H), 7.68 (br s, 1H), 7.95 (t, 1H), 8.42 (d, 1H), 9.10 (s, 1H), 10.46 (s, 1H). |
| | RCO₂H: 3-fluoro-7-isopropoxy-2-(1-methyl-2-oxabicyclo[2.1.1]hexan-4- | |
| | yl)imidazo[1,2-a]pyridine-6-carboxylic acid (Preparation 319) | |
| | R-NH₂: 6-(difluoromethyl)pyridin-2-amine hydrochloride. prep-HPLC-D | |
| 325 | 3-Fluoro-7-isopropoxy-N-(2-methoxypyridin-3-yl)-2-(1-methyl-2-oxa- | 9.10 mg, 9.95% yield |
| | bicyclo[2.1.1]hexan-4-yl)imidazo[1,2-a]pyridine-6-carboxamide trifluoroacetate | LCMS m/z = 441.2 [M + H]+ |
| | | ¹H NMR (500 MHz, |
| | 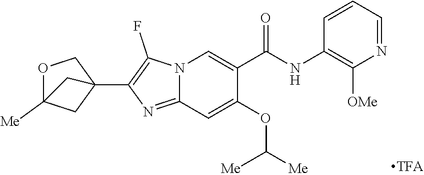 [SMILES: COc1ncccc1NC(=O)c1cn2c(F)c(C34COC(C)(C3)C4)nc2cc1OC(C)C] | DMSO-d₆) δ: 1.44 (s, 3H), 1.49 (d, 6H), 1.81 (dd, 2H), 2.09 (dd, 2H), 3.95 (s, 2H), 4.02 (s, 3H), 5.00-5.10 (m, 1H), 7.08 (dd, 1H), 7.23 (s, 1H), 7.93 (br d, 1H), 7.95 (dd, 1H) 8.70-8.73 (m, 1H), 8.76 (s, 1H), 10.26 (s, 1H). |
| | RCO₂H: 3-fluoro-7-isopropoxy-2-(1-methyl-2-oxabicyclo[2.1.1]hexan-4- | |
| | yl)imidazo[1,2-a]pyridine-6-carboxylic acid (Preparation 319) | |
| | R-NH₂: 2-methoxypyridin-3-amine | |
| | prep-HPLC-D | |
| 326 | N-(1-(difluoromethyl)-2-oxo-1,2-dihydropyridin-3-yl)-3-fluoro-7-isopropoxy-2- | 84.6 mg, 91.0% yield |
| | (1-methyl-2-oxabicyclo[2.1.1]hexan-4-yl)imidazo[1,2-a]pyridine-6-carboxamide | LCMS m/z = 477.2 [M + H]⁺ |
| | | ¹H NMR (400 MHz, CDCl₃) |
| | 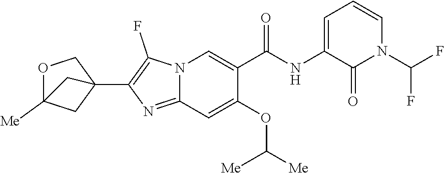 [SMILES: CC(C)Oc1cc2nc(C34COC(C)(C3)C4)c(F)n2cc1C(=O)Nc1cccn(C(F)F)c1=O] | δ: 1.56 (s, 3H), 1.62 (d, 6H), 1.96-2.03 (m, 2H), 2.18 (br s, 2H), 4.14 (s, 2H), 4.84 (spt, 1H), 6.43 (t, 1H), 6.89- 7.01 (m, 1H), 7.23-7.27 (m, 1H), 7.61-7.98 (m, 1H), 8.63 (dd, 1H), 8.85 (s, 1H), 10.77 (s, 1H). |
| | RCO₂H: 3-fluoro-7-isopropoxy-2-(1-methyl-2-oxabicyclo[2.1.1]hexan-4- | |
| | yl)imidazo[1,2-a]pyridine-6-carboxylic acid (Preparation 319) | |
| | R-NH₂: 3-amino-1-(difluoromethyl)pyridin-2(1H)-one. SiO2; (3:1 EtOAc/EtOH)/ | |
| | Heptane | |
| 327 | 3-Fluoro-7-isopropoxy-N-(1-methyl-1H-pyrazol-3-yl)-2-(1-methyl-2- | LCMS m/z = 414.2 [M + H]+ |
| | oxabicyclo[2.1.1]hexan-4-yl)imidazo[1,2-a]pyridine-6-carboxamide | ¹H NMR (400 MHz, CDCl₃) |
| | trifluoroacetate | δ: 1.55-1.63 (m, 9H), 2.06 |
| | | (dd, 2H), 2.27-2.37 (m, 2H), |
| | 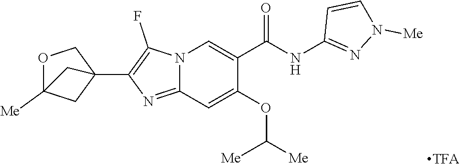 [SMILES: CC(C)Oc1cc2nc(C34COC(C)(C3)C4)c(F)n2cc1C(=O)Nc1ccn(C)n1] | 3.94 (s, 3H), 4.18 (s, 2H), 5.04 (spt, 1H), 6.87 (d, 1H), 7.41 (d, 1H), 7.62 (s, 1H), 8.94-9.04 (m, 1H), 8.97-9.02 (m, 1H), 10.19 (s, 1H). |
| | RCO₂H: 3-fluoro-7-isopropoxy-2-(1-methyl-2-oxabicyclo[2.1.1]hexan-4-yl) | |
| | imidazo[1,2-a]pyridine-6-carboxylic acid (Preparation 319) | |
| | R-NH₂: 1-methylpyrazole-3-amine | |
| | prep-HPLC-D | |
| 328 | 3-Fluoro-7-isopropoxy-2-(1-methyl-2-oxabicyclo[2.1.1]hexan-4-yl)-N- | 63.7 mg, 72.44% yield |
| | (pyrazolo[1,5-a]pyrimidin-3-yl)imidazo[1,2-a]pyridine-6-carboxamide | LCMS m/z = 451.2 [M + H]⁺ |
| | | ¹H NMR (500 MHz, CDCl₃) |
| | 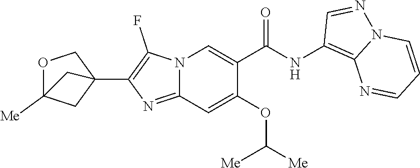 [SMILES: CC(C)Oc1cc2nc(C34COC(C)(C3)C4)c(F)n2cc1C(=O)Nc1cnn2cccnc12] | δ: 1.56 (s, 3H), 1.68 (d, 6H), 1.97-2.03 (m, 2H), 2.17-2.24 (m, 2H), 4.15 (s, 2H), 4.90 (spt, 1H), 6.84 (dd, 1H), 7.00 (br s, 1H), 8.39-8.47 (m, 1H), 8.61-8.68 (m, 1H), 8.97 (d, 2H), 10.55 (s, 1H). |
| | RCO₂H: 3-fluoro-7-isopropoxy-2-(1-methyl-2-oxabicyclo[2.1.1]hexan-4- | |
| | yl)imidazo[1,2-a]pyridine-6-carboxylic acid (Preparation 319) | |
| | R-NH₂: pyrazolo[1,5-a]pyrimidin-3-amine SiO2; (3:1 EtOAc/EtOH)/Heptane | |
| 329 | N-([1,2,4]triazolo[1,5-a]pyridin-5-yl)-3-fluoro-7-isopropoxy-2-(1-methyl-2- | 5.30 mg, 7.15% yield |
| | oxabicyclo[2.1.1]hexan-4-yl)imidazo[1,2-a]pyridine-6-carboxamide | LCMS m/z = 451.2 [M + H]⁺ |
| | trifluoroactate | ¹H NMR (500 MHz, |
| | | DMSO-d6) δ: 1.45 (s, 3H), |
| | 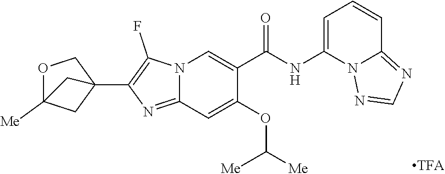 [SMILES: CC(C)Oc1cc2nc(C34COC(C)(C3)C4)c(F)n2cc1C(=O)Nc1cccc2ncnn12] | 1.60 (d, 6H), 1.81 (dd, 2H), 2.09 (dd, 2H), 3.95 (s, 2H), 5.13 (spt, 1H), 7.31 (d, 1H), 7.63-7.68 (m, 1H), 7.80 (dd, 1H), 8.07 (d, 1H), 8.60-8.73 (m, 1H), 8.84 (s, 1H), 11.54 (s, 1H). |
| | RCO₂H: 3-fluoro-7-isopropoxy-2-(1-methyl-2-oxabicyclo[2.1.1]hexan-4- | |
| | yl)imidazo[1,2-a]pyridine-6-carboxylic acid (Preparation 319) | |
| | R-NH₂: [1,2,4]triazo[1,5-a]pyridine-5-amine | |
| | prep-HPLC-D | |
| 330 | N-(6-(difluoromethyl)pyridin-2-yl)-2-(1,4-dioxan-2-yl)-7-isopropoxy- | 15.1 mg, 29.7% yield |
| | imidazo[1,2-a]pyridine-6-carboxamide | LCMS m/z = 433.2 [M + H]⁺ |
| | | ¹H NMR (500 MHz, |
| | 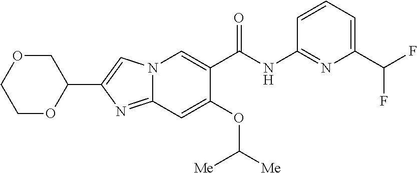 [SMILES: CC(C)Oc1cc2nc(C3COCCO3)cn2cc1C(=O)Nc1cccc(C(F)F)n1] | DMSO-d₆) δ: 1.43 (dd, 6H), 3.51-3.62 (m, 2H), 3.72-3.81 (m, 2H), 3.84-3.89 (m, 1H), 3.99 (dd, 1H), 4.70 (dd, 1H), 4.94 (spt, 1H), 6.73-7.00 (m, 1H), 7.15 (s, 1H), 7.47 (d, 1H), 7.85 (s, 1H), 8.08 (t, 1H), 8.36 (br d, 1H), 9.15 (s, 1H), 10.87 (s, 1H). |
| | RCO₂H: 2-(1,4-dioxan-2-yl)-7-isopropoxyimidazo[1,2-a]pyridine-6-carboxylic | |
| | acid (Preparation 349) | |
| | R-NH₂: 6-(difluoromethyl)pyridin-2-amine hydrochloride. prep-HPLC-F | |
| 331 | 2-(1,4-Dioxan-2-yl)-7-isopropoxy-N-(pyrazolo[1,5-a]pyrimidin-3- | 11.50 mg, 23.16% yield |
| | yl)imidazo[1,2-a]pyridine-6-carboxamide | LCMS m/z = 432.2 [M + H]⁺ |
| | | ¹H NMR (500 MHz, |
| | 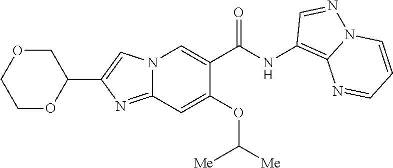 [SMILES: CC(C)Oc1cc2nc(C3COCCO3)cn2cc1C(=O)Nc1cnn2cccnc12] | DMSO-d₆) δ: 1.54 (dd, 6H), 3.48-3.64 (m, 2H), 3.71-3.81 (m, 2H), 3.83-3.91 (m, 1H), 4.00 (dd, 1H), 4.72 (dd, 1H), 5.05 (quin, 1H), 7.07 (dd, 1H), 7.17-7.27 (m, 1H), 7.91 (s, 1H), 8.52-8.58 (m, 1H), 8.76 (s, 1H), 9.07-9.13 (m, 1H), 9.27 (s, 1H), 10.51 (s, 1H). |
| | RCO₂H: 2-(1,4-dioxan-2-yl)-7-isopropoxyimidazo[1,2-a]pyridine-6-carboxylic | |
| | acid (Preparation 349) | |
| | R-NH₂: pyrazolo[1,5-a]pyrimidin-3-amine | |
| | prep-HPLC-F | |
| 332 | 7-(Difluoromethoxy)-N-(2-methoxypyridin-3-yl)-2-(1-methyl-2-oxabicyclo | 61 mg, 51.9% yield |
| | [2.1.1]hexan-4-yl)imidazo[1,2-a]pyridine-6-carboxamide trifluoroacetate | LCMS m/z = 431.2 [M + H]⁺ |
| | | ¹H NMR (500 MHz, |
| | 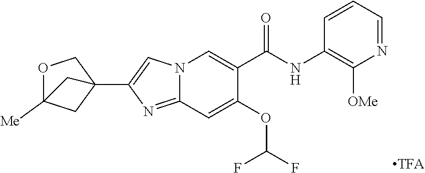 [SMILES: COc1ncccc1NC(=O)c1cn2cc(C34COC(C)(C3)C4)nc2cc1OC(F)F] | DMSO-d₆) δ: 1.44 (s, 3H), 1.81 (dd, 2H), 2.03-2.09 (m, 2H), 3.92 (s, 2H), 3.97 (s, 3H), 7.07 (dd, 1H), 7.38- 7.69 (m, 2H), 7.97 (dd, 1H), 8.01 (s, 1H), 8.52 (br d, 1H), 9.20 (s, 1H), 9.91 (s, 1H). |
| | RCO₂H: 7-(difluoromethoxy)-2-(1-methyl-2-oxabicyclo[2.1.1]hexan-4- | |
| | yl)imidazo[1,2-a]pyridine-6-carboxylic acid (Preparation 317) | |
| | R-NH₂: 2-methoxypyridin-3-amine | |
| | prep-HPLC-D | |
| 333 | 7-(Difluoromethoxy)-N-(6-(difluoromethyl)pyridin-2-yl)-2-(1-methyl-2- | 39.9 mg, 32.75% yield |
| | oxabicyclo[2.1.1]hexan-4-yl)imidazo[1,2-a]pyridine-6-carboxamide | LCMS m/z = 451.1 [M + H]⁺ |
| | trifluoroacetate | ¹H NMR (500 MHz, |
| | | DMSO-d₆) δ: 1.44 (s, 3H), |
| | 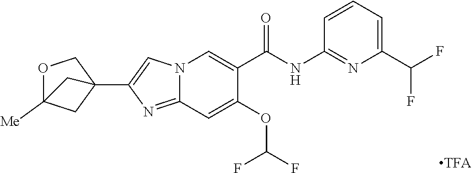 [SMILES: CC12CC(c3cn4cc(C(=O)Nc5cccc(C(F)F)n5)c(OC(F)F)cc4n3)(CO1)C2] | 1.80 (dd, 2H), 2.02-2.07 (m, 2H), 3.92 (s, 2H), 6.82-7.22 (m, 2H), 7.32-7.40 (m, 1H), 7.46-7.55 (m, 1H), 7.93 (s, 1H), 8.09 (t, 1H), 8.34 (br d, 1H), 9.03 (s, 1H), 11.30 (s, 1H). |
| | RCO₂H: 7-(difluoromethoxy)-2-(1-methyl-2-oxabicyclo[2.1.1]hexan-4- | |
| | yl)imidazo[1,2-a]pyridine-6-carboxylic acid (Preparation 317) | |
| | R-NH₂: 6-(difluoromethyl)pyridin-2-amine hydrochloride. prep-HPLC-D | |
| 334 | N-(6-(Difluoromethyl)pyridin-2-yl)-2-(1-methyl-2-oxabicyclo[2.1.1]hexan-4- | 11.5 mg, 14.07% yield |
| | yl)imidazo[1,2-a]pyrazine-6-carboxamide | LCMS m/z = 386.2 [M + H]⁺ |
| | | ¹H NMR (500 MHz, |
| | 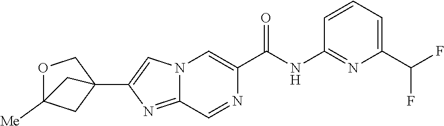 [SMILES: CC12CC(c3cn4cc(C(=O)Nc5cccc(C(F)F)n5)ncc4n3)(CO1)C2] | DMSO-d₆) δ: 1.45 (s, 3H), 1.81-1.90 (m, 2H), 2.04-2.16 (m, 2H), 3.97 (s, 2H), 6.77- 7.08 (m, 1H), 7.52 (d, 1H), 8.13 (t, 1H), 8.27 (s, 1H), 8.42 (d, 1H), 9.09-9.46 (m, 1H), 10.41 (s, 1H). |
| | RCO₂H: 2-(1-methyl-2-oxabicyclo[2.1.1]hexan-4-yl)imidazo[1,2-a]pyrazine- | |
| | 6-carboxylic acid (Preparation 350). | |
| | R-NH₂: 6-(difluoromethyl)pyridin-2-amine hydrochloride. prep-HPLC-F | |
| 335 | N-(6-(difluoromethyl)pyridin-2-yl)-8-propoxy-2-(tetrahydro-2H-pyran-4- | 21.80 mg, 30.55% yield |
| | yl)imidazo[1,2-a]pyrazine-6-carboxamide | LCMS m/z = 432.2 [M + H]⁺ |
| | | ¹H NMR (500 MHz, |
| | 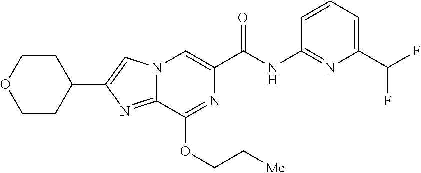 [SMILES: CCCOc1nc(C(=O)Nc2cccc(C(F)F)n2)cn2cc(C3CCOCC3)nc12] | DMSO-d₆) δ: 1.08 (t, 3H), 1.64-1.80 (m, 2H), 1.87-1.98 (m, 4H), 2.97-3.05 (m, 1H), 3.40-3.52 (m, 2H), 3.95 (dt, 2H), 4.61 (t, 2H), 6.82-7.10 (m, 1H), 7.52 (d, 1H), 8.05 (s, 1H), 8.12 (t, 1H), 8.41 (d, 1H), 9.03 (s, 1H), 10.17 (s, 1H). |
| | RCO₂H: 8-propoxy-2-(tetrahydro-2H-pyran-4-yl)imidazo[1,2-a]pyrazine-6- | |
| | carboxylic acid (Preparation 367). | |
| | R-NH₂: 6-(difluoromethyl)pyridin-2-amine hydrochloride. prep-HPLC-F | |
| 336 | N-(6-(difluoromethyl)pyridin-2-yl)-8-isopropoxy-2-(1-methyl-2- | 22.5 mg, 12.4% yield |
| | oxabicyclo[2.1.1]hexan-4-yl)imidazo[1,2-a]pyrazine-6-carboxamide | LCMS m/z = 443.2 [M + H]⁺ |
| | | ¹H NMR (500 MHz, |
| | 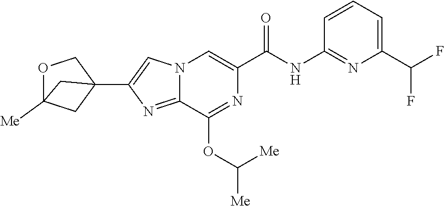 [SMILES: CC(C)Oc1nc(C(=O)Nc2cccc(C(F)F)n2)cn2cc(C34COC(C)(C3)C4)nc12] | DMSO-d₆) δ: 1.44 (s, 3H), 1.48 (d, 6H), 1.77-1.85 (m, 2H), 2.00-2.11 (m, 2H), 3.92 (s, 2H), 5.74 (spt, 1H), 6.81- 7.07 (m, 1H), 7.53 (d, 1H), 8.09-8.16 (m, 2H), 8.40 (d, 1H), 9.00-9.03 (m, 1H), 10.17 (s, 1H). |
| | RCO₂H: 8-isopropoxy-2-(1-methyl-2-oxabicyclo[2.1.1]hexan-4-yl)imidazo[1,2- | |
| | a]pyrazine-6-carboxylic acid (Preparation 305) | |
| | R-NH₂: 6-(difluoromethyl)pyridin-2-amine hydrochloride. prep-HPLC-F | |
| 337 | 8-Isopropoxy-N-(2-methoxypyridin-3-yl)-2-(1-methyl-2-oxabicyclo[2.1.1]hexan- | 9.40 mg, 17.31% yield |
| | 4-yl)imidazo[1,2-a]pyrazine-6-carboxamide | LCMS m/z = 424.3 [M + H]⁺ |
| | | ¹H NMR (500 MHz, |
| | 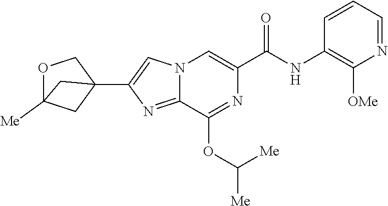 [SMILES: COc1ncccc1NC(=O)c1cn2cc(C34COC(C)(C3)C4)nc2c(OC(C)C)n1] | DMSO-d₆) δ: 1.44 (s, 3H), 1.54 (d, 6H), 1.77-1.85 (m, 2H), 2.01-2.09 (m, 2H), 3.92 (s, 2H), 4.03 (s, 3H), 5.55 (spt, 1H), 6.96-7.21 (m, 1H), 7.94 (dd, 1H), 8.12 (s, 1H), 8.63 (dd, 1H), 8.95 (s, 1H), 10.15 (s, 1H). |
| | RCO₂H: 8-isopropoxy-2-(1-methyl-2-oxabicyclo[2.1.1]hexan-4-yl)imidazo[1,2- | |
| | a]pyrazine-6-carboxylic acid (Preparation 305) | |
| | R-NH₂: 2-methoxypyridin-3-amine | |
| | prep-HPLC-F | |
| 338 | N-(6-(difluoromethyl)pyridin-2-yl)-2-(1-(fluoromethyl)-2-oxabicyclo[2.1.1] | 5.20 mg, 8.79% yield |
| | hexan-4-yl)-8-isopropoxyimidazo[1,2-a]pyrazine-6-carboxamide | LCMS m/z = 462.1 [M + H]⁺ |
| | | ¹H NMR (500 MHz, |
| | 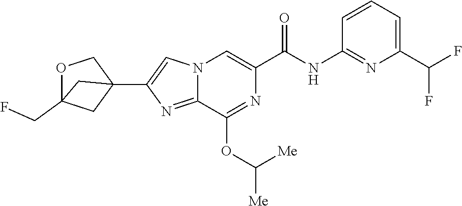 [SMILES: CC(C)Oc1nc(C(=O)Nc2cccc(C(F)F)n2)cn2cc(C34COC(CF)(C3)C4)nc12] | DMSO-d₆) δ: 1.49 (d, 6H), 1.91 (dd, 2H), 2.18-2.25 (m, 2H), 4.01 (s, 2H), 4.62-4.78 (m, 2H), 5.71-5.82 (m, 1H), 6.83-7.08 (m, 1H), 7.53 (d, 1H), 8.03-8.16 (m, 1H), 8.17 (s, 1H) 8.40 (d, 1H), 9.03 (s, 1H), 10.18 (s, 1H). |
| | RCO₂H: 2-(1-(fluoromethyl)-2-oxabicyclo[2.1.1]hexan-4-yl)-8- | |
| | isopropoxyimidazo[1,2-a]pyrazine-6-carboxylic acid (Preparation 338) | |
| | R-NH₂: 6-(difluoromethyl)pyridin-2-amine hydrochloride. prep-HPLC-F | |
| 339 | 2-(1-(Fluoromethyl)-2-oxabicyclo[2.1.1]hexan-4-yl)-8-isopropoxy-N- | 5.0 mg, 8.83% yield |
| | (2-methoxypyridin-3-yl)imidazo[1,2-a]pyrazine-6-carboxamide | LCMS m/z = 442.2 [M + H]⁺ |
| | | ¹H NMR (500 MHz, |
| | 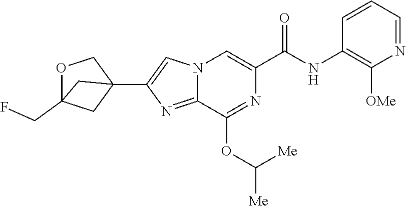 [SMILES: COc1ncccc1NC(=O)c1cn2cc(C34COC(CF)(C3)C4)nc2c(OC(C)C)n1] | DMSO-d6) δ: 1.55 (d, 6H), 1.91 (dd, 2H), 2.19-2.24 (m, 2H), 4.01 (s, 2H), 4.04 (s, 3H), 4.65-4.80 (m, 2H), 5.51-5.59 (m, 1H), 7.06-7.11 (m, 1H), 7.95 (dd, 1H), 8.17 (s, 1H), 8.63 (dd, 1H), 8.96 (s, 1H) 10.15 (s, 1H) |
| | RCO₂H: 2-(1-(fluoromethyl)-2-oxabicyclo[2.1.1]hexan-4-yl)-8- | |
| | isopropoxyimidazo[1,2-a]pyrazine-6-carboxylic acid (Preparation 338) | |
| | R-NH₂: 2-methoxypyridin-3-amine | |
| | prep-HPLC-F | |
| 340 | 2-(1-(Fluoromethyl)-2-oxabicyclo[2.1.1]hexan-4-yl)-8-isopropoxy-N- | 15.70 mg, 27.1% yield |
| | (pyrazolo[1,5-a]pyrimidin-3-yl)imidazo[1,2-a]pyrazine-6-carboxamide | LCMS m/z = 452.2 [M + H]⁺ |
| | | ¹H NMR (500 MHz, |
| | 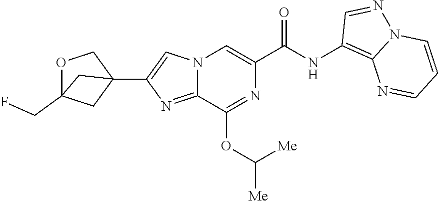 [SMILES: CC(C)Oc1nc(C(=O)Nc2cnn3cccnc23)cn2cc(C34COC(CF)(C3)C4)nc12] | DMSO-d6) δ: 1.48 (d, 6H), 1.86-1.96 (m, 2H), 2.21 (dd, 2H), 4.01 (s, 2H), 4.63-4.80 (m, 2H), 5.79-5.90 (m, 1H), 7.09 (dd, 1H), 8.17 (s, 1H), 8.54-8.62 (m, 2H), 8.93 (s, 1H), 9.06-9.17 (m, 1H), 9.97 (s, 1H) |
| | RCO₂H: 2-(1-(fluoromethyl)-2-oxabicyclo[2.1.1]hexan-4-yl)-8- | |
| | isopropoxyimidazo[1,2-a]pyrazine-6-carboxylic acid (Preparation 338) | |
| | R-NH₂: pyrazolo[1,5-a]pyrimidin-3-amine | |
| | prep-HPLC-F | |
| 341 | 8-Isopropoxy-N-(1-methyl-2-oxo-1,2-dihydropyridin-3-yl)-2-(1-methyl-2- | 10.8 mg, 60.54% yield |
| | oxabicyclo[2.2.1]heptan-4-yl)imidazo[1,2-a]pyrazine-6-carboxamide | LCMS m/z = 438.2 [M + H]⁺ |
| | | ¹H NMR (400 MHz, CDCl₃) |
| | 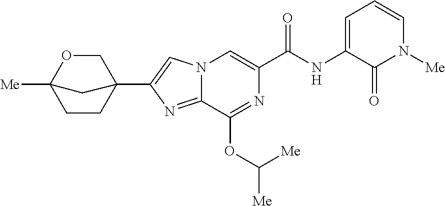 [SMILES: CC(C)Oc1nc(C(=O)Nc2cccn(C)c2=O)cn2cc(C34CCC(C)(C3)OC4)nc12] | δ: 1.40 (s, 3H), 1.54 (d, 6H), 1.71-1.82 (m, 2H), 1.86-1.92 (m, 1H), 1.95-2.00 (m, 1H), 2.01-2.15 (m, 2H), 3.59 (s, 3H), 3.92 (d, 1H), 4.05 (dd, 1H), 5.61-5.72 (m, 1H), 6.22 (t, 1H), 6.98 (dd, 1H), 7.44 (s, 1H), 8.46-8.56 (m, 2H), 10.50 (s, 1H) |
| | RCO₂H: 8-isopropoxy-2-(1-methyl-2-oxabicyclo[2.2.1]heptan-4- | |
| | yl)imidazo[1,2-a]pyrazine-6-carboxylic acid (Preparation 353) | |
| | R-NH₂: 3-amino-1-methylpyridin-2-one | |
| | prep-HPLC-F | |
| 342 | N-(2-methoxypyridin-3-yl)-2-(1-methyl-2-oxabicyclo[2.1.1]hexan-4-yl)-8- | 12.20 mg, 18.29% yield |
| | propoxyimidazo[1,2-a]pyrazine-6-carboxamide | LCMS m/z = 424.4 [M + H]⁺ |
| | | ¹H NMR (500 MHz, |
| | 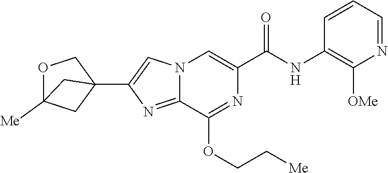 [SMILES: CCCOc1nc(C(=O)Nc2cccnc2OC)cn2cc(C34COC(C)(C3)C4)nc12] | DMSO-d₆) δ: 1.08 (t, 3H), 1.44 (s, 3H), 1.81 (dd, 2H), 1.91-2.01 (m, 2H), 2.06 (dd, 2H), 3.93 (s, 2H), 4.03 (s, 3H), 4.59 (t, 2H), 7.09 (dd, 1H), 7.95 (dd, 1H), 8.11- 8.15 (m, 1H), 8.62 (dd, 1H), 8.97 (s, 1H), 10.12 (s, 1H) |
| | RCO₂H: 2-(1-methyl-2-oxabicyclo[2.1.1]hexan-4-yl)-8-propoxyimidazo[1,2- | |
| | a]pyrazine-6-carboxylic acid (Preparation 337) | |
| | R-NH₂: 2-methoxypyridin-3-amine | |
| | prep-HPLC-F | |
| 343 | 2-(1-Methyl-2-oxabicyclo[2.1.1]hexan-4-yl)-8-propoxy-N-(6- | 22.4 mg, 51.4% yield |
| | (trifluoromethyl)pyridin-2-yl)imidazo[1,2-a]pyrazine-6-carboxamide | LCMS m/z = 462.1 [M + H]⁺ |
| | | ¹H NMR (500 MHz, |
| | 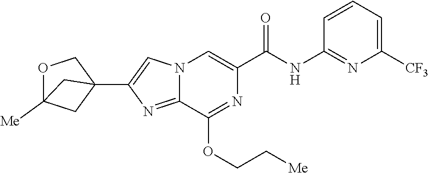 [SMILES: CCCOc1nc(C(=O)Nc2cccc(C(F)(F)F)n2)cn2cc(C34COC(C)(C3)C4)nc12] | DMSO-d₆) δ: 1.07 (t, 3H), 1.44 (s, 3H), 1.77-1.84 (m, 2H), 1.89-1.97 (m, 2H), 2.07 (dd, 2H), 3.93 (s, 2H), 4.62 (t, 2H), 7.73 (d, 1H), 8.11- 8.15 (m, 1H), 8.21 (t, 1H), 8.51 (d, 1H), 9.04 (s, 1H), 10.30 (s, 1H) |
| | RCO₂H: 2-(1-methyl-2-oxabicyclo[2.1.1]hexan-4-yl)-8-propoxyimidazo[1,2- | |
| | a]pyrazine-6-carboxylic acid (Preparation 337). | |
| | R-NH₂: 6-(trifluoromethyl)pyridine-2-amine | |
| | prep-HPLC-F | |
| 344 | 8-(2,2-Difluoroethoxy)-N-(6-(difluoromethyl)pyridin-2-yl)-2-(1-methyl-2- | 35.3 mg, 25.7% yield |
| | oxabicyclo[2.1.1]hexan-4-yl)imidazo[1,2-a]pyrazine-6-carboxamide | LCMS m/z = 466.2 [M + H]⁺ |
| | | ¹H NMR (500 MHz, |
| | 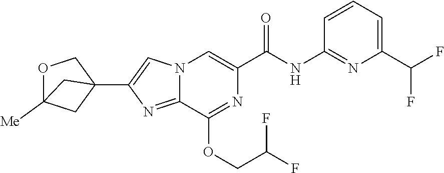 [SMILES: CC12CC(c3cn4cc(C(=O)Nc5cccc(C(F)F)n5)nc(OCC(F)F)c4n3)(CO1)C2] | DMSO-d₆) δ: 1.44 (s, 3H), 1.82 (dd, 2H), 2.08 (dd, 2H), 3.94 (s, 2H), 5.07 (td, 2H), 6.50-6.74 (m, 1H), 6.83-7.11 (m, 1H), 7.53 (d, 1H), 8.19 (s, 1H), 8.37 (d, 1H), 9.10 (s, 1H), 10.51 (s, 1H) |
| | RCO₂H: 8-(2,2-difluoroethoxy)-2-(1-methyl-2-oxabicyclo[2.1.1]hexan-4- | |
| | yl)imidazo[1,2-a]pyrazine-6-carboxylic acid (Preparation 336) | |
| | R-NH₂: 6-(difluoromethyl)pyridin-2-amine hydrochloride. prep-HPLC-F | |
| 345 | 8-Cyclobutoxy-N-(6-(difluoromethyl)pyridin-2-yl)-2-(1-methyl-2-oxabicyclo | 6.40 mg, 5.44% yield |
| | [2.1.1]hexan-4-yl)imidazo[1,2-a]pyrazine-6-carboxamide | LCMS m/z = 456.4 [M + H]⁺ |
| | | ¹H NMR (500 MHz, |
| | 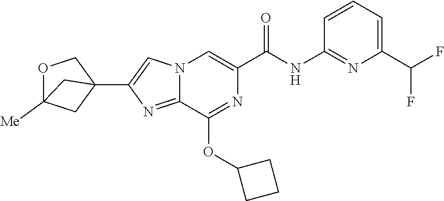 [SMILES: CC12CC(c3cn4cc(C(=O)Nc5cccc(C(F)F)n5)nc(OC5CCC5)c4n3)(CO1)C2] | DMSO-d₆) δ: 1.44 (s, 3H), 1.76-1.86 (m, 3H), 1.89-1.98 (m, 1H), 2.03-2.09 (m, 2H), 2.27-2.40 (m, 2H), 2.57-2.64 (m, 2H), 3.93 (s, 2H), 5.48- 5.56 (m, 1H), 6.83-7.09 (m, 1H), 7.53 (d, 1H), 8.07-8.16 (m, 2H), 8.39 (d, 1H), 9.03 (s, 1H), 10.13 (s, 1H). |
| | RCO₂H: 8-cyclobutoxy-2-(1-methyl-2-oxabicyclo[2.1.1]hexan-4-yl)imidazo[1,2- | |
| | a]pyrazine-6-carboxylic acid (Preparation 339). | |
| | R-NH₂: 6-(difluoromethyl)pyridin-2-amine hydrochloride. prep-HPLC-F | |
| 346 | 8-Cyclobutoxy-N-(1-methyl-1H-pyrazol-3-yl)-2-(1-methyl-2-oxabicyclo[2.1.1] | 20.30 mg, 27.28% yield |
| | hexan-4-yl)imidazo[1,2-a]pyrazine-6-carboxamide | LCMS m/z = 409.3 [M + H]⁺ |
| | | ¹H NMR (500 MHz, |
| | 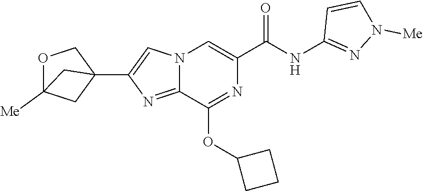 [SMILES: Cn1ccc(NC(=O)c2cn3cc(C45COC(C)(C4)C5)nc3c(OC3CCC3)n2)n1] | DMSO-d₆) δ: 1.44 (s, 3H), 1.72-1.84 (m, 3H), 1.84-1.94 (m, 1H), 2.02-2.09 (m, 2H), 2.20-2.32 (m, 2H), 2.53-2.57 (m, 2H), 3.81 (s, 3H), 3.92 (s, 2H), 5.60-5.71 (m, 1H), 6.60 (d, 1H), 7.66 (d, 1H), 8.11 (s, 1H), 8.90 (s, 1H), 10.04 (s, 1H) |
| | RCO₂H: 8-cyclobutoxy-2-(1-methyl-2-oxabicyclo[2.1.1]hexan-4-yl)imidazo[1,2- | |
| | a]pyrazine-6-carboxylic acid (Preparation 339). | |
| | R-NH₂: 1-methylpyrazole-3-amine | |
| | prep-HPLC-F | |
| 347 | 8-Cyclobutoxy-N-(2-methoxypyridin-3-yl)-2-(1-methyl-2-oxabicyclo[2.1.1] | 6.90 mg, 6.14% yield |
| | hexan-4-yl)imidazo[1,2-a]pyrazine-6-carboxamide | LCMS m/z = 436.2 [M + H]⁺ |
| | | ¹H NMR (500 MHz, |
| | 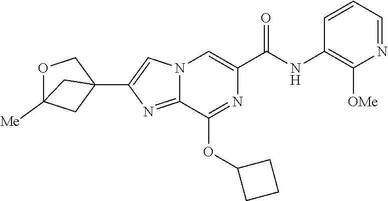 [SMILES: COc1ncccc1NC(=O)c1cn2cc(C34COC(C)(C3)C4)nc2c(OC2CCC2)n1] | DMSO-d₆) δ: 1.44 (s, 3H), 1.77-1.90 (m, 3H), 1.89-2.01 (m, 1H), 2.02-2.11 (m, 2H), 2.29-2.39 (m, 2H), 2.65-2.70 (m, 2H), 3.92 (s, 2H), 4.05 (s, 3H), 5.32-5.46 (m, 1H), 7.09 (dd, 1H), 7.94 (dd, 1H), 8.10-8.16 (m, 1H), 8.67 (dd, 1H), 8.97 (s, 1H), 10.11 (s, 1H) |
| | RCO₂H: 8-cyclobutoxy-2-(1-methyl-2-oxabicyclo[2.1.1]hexan-4-yl)imidazo[1,2- | |
| | a]pyrazine-6-carboxylic acid (Preparation 339). | |
| | R-NH₂: 2-methoxypyridin-3-amine | |
| | prep-HPLC-F | |
| 348 | 8-Cyclobutoxy-2-(1-methyl-2-oxabicyclo[2.1.1]hexan-4-yl)-N-(pyrazolo[1,5- | 2.80 mg, 3.46% yield |
| | a]pyridin-7-yl)imidazo[1,2-a]pyrazine-6-carboxamide | LCMS m/z = 445.0 [M + H]⁺ |
| | | ¹H NMR (500 MHz, |
| | 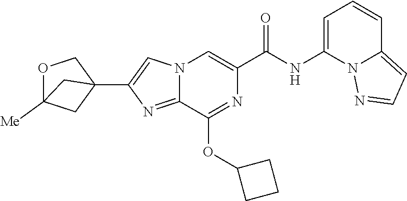 [SMILES: CC12CC(c3cn4cc(C(=O)Nc5cccc6ccnn56)nc(OC5CCC5)c4n3)(CO1)C2] | DMSO-d₆) δ: 1.45 (s, 3H), 1.78-1.85 (m, 2H), 1.86-1.99 (m, 2H), 2.04-2.10 (m, 2H), 2.29-2.38 (m, 2H), 2.81-2.92 (m, 2H), 3.94 (s, 2H), 5.45- 5.56 (m, 1H), 6.76 (d, 1H), 7.38 (dd, 1H), 7.47-7.59 (m, 1H), 7.74-7.84 (m, 1H), 8.08-8.23 (m, 2H), 9.08 (s, 1H), 11.65 (s, 1H) |
| | RCO₂H: 8-cyclobutoxy-2-(1-methyl-2-oxabicyclo[2.1.1]hexan-4-yl)imidazo[1,2- | |
| | a]pyrazine-6-carboxylic acid (Preparation 339). | |
| | R-NH₂: pyrazolo[1,5-a]pyridin-7amine | |
| | prep-HPLC-F | |
| 349 | 8-Cyclobutoxy-2-(1-methyl-2-oxabicyclo[2.1.1]hexan-4-yl)-N-(pyrazolo[1,5- | 20 mg, 24.64% yield |
| | a]pyrimidin-3-yl)imidazo[1,2-a]pyrazine-6-carboxamide | LCMS m/z = 446.3 [M + H]⁺ |
| | | ¹H NMR (500 MHz, |
| | 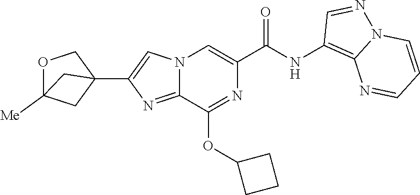 [SMILES: CC12CC(c3cn4cc(C(=O)Nc5cnn6cccnc56)nc(OC5CCC5)c4n3)(CO1)C2] | DMSO-d₆) δ: 1.45 (s, 3H), 1.73-1.86 (m, 3H), 1.88-1.98 (m, 1H), 2.03-2.11 (m, 2H), 2.23-2.38 (m, 2H), 2.62-2.71 (m, 2H), 3.93 (s, 2H), 5.53- 5.62 (m, 1H), 7.09 (dd, 1H), 8.13 (s, 1H), 8.56-8.67 (m, 2H), 8.94 (s, 1H), 9.09-9.18 (m, 1H), 9.93 (s, 1H) |
| | RCO₂H: 8-cyclobutoxy-2-(1-methyl-2-oxabicyclo[2.1.1]hexan-4-yl)imidazo[1,2- | |
| | a]pyrazine-6-carboxylic acid (Preparation 339). | |
| | R-NH₂: pyrazolo[1,5-a]pyrimidin-3-amine. prep-HPLC-F | |
| 350 | 7-(Methoxy methyl)-N-(1-methyl-1H-pyrazol-3-yl)-2-(1-methyl-2- | 3.40 mg, 10.37% yield |
| | oxabicyclo[2.1.1]hexan-4-yl)imidazo[1,2-a]pyrimidine-6-carboxamide | LCMS m/z = 383.2 [M + H]⁺ |
| | | ¹H NMR (500 MHz, |
| | 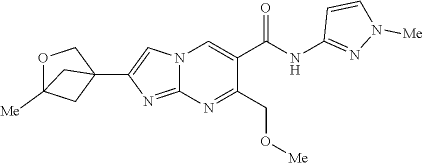 [SMILES: COCc1nc2nc(C34COC(C)(C3)C4)cn2cc1C(=O)Nc1ccn(C)n1] | DMSO-d₆) δ: 1.45 (s, 3H), 1.78-1.86 (m, 2H), 2.07 (dd, 2H), 3.26 (s, 3H) 3.78 (s, 3H), 3.93 (s, 2H), 4.69 (s, 2H), 6.58 (d, 1H), 7.63 (d, 1H), 7.87 (s, 1H), 9.18 (s, 1H), 9.17-9.17 (m, 1H), 11.02 (s, 1 H) |
| | RCO₂H: 7-(methoxymethyl)-2-(1-methyl-2-oxabicyclo[2.1.1]hexan-4- | |
| | yl)imidazo[1,2-a]pyrimidine-6-carboxylic acid (Preparation 331). | |
| | R-NH₂: 1-methylpyrazol-3-amine | |
| | prep-HPLC-F | |
| 351 | 7-(Methoxymethyl)-N-(1-methyl-2-oxo-1,2-dihydropyridin-3-yl)-2-(1- | 8.90 mg, 25.36% yield |
| | methyl-2-oxabicyclo[2.1.1]hexan-4-yl)imidazo[1,2-a]pyrimidine-6-carboxamide | LCMS m/z = 410.2 [M + H]⁺ |
| | | ¹H NMR (500 MHz, |
| | 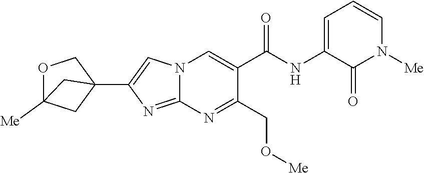 [SMILES: COCc1nc2nc(C34COC(C)(C3)C4)cn2cc1C(=O)Nc1cccn(C)c1=O] | DMSO-d₆) δ: 1.45 (s, 4H), 1.78-1.86 (m, 2H), 2.04-2.11 (m, 2H), 3.30 (s, 3H) 3.49- 3.59 (m, 2H), 3.93 (s, 2H), 4.68 (s, 2H), 6.33 (t, 1H), 7.51 (dd, 1H), 7.88 (s, 1H), 8.34 (d, 1H), 9.24 (s, 1H), 9.99 (s, 1H) |
| | RCO₂H: 7-(methoxymethyl)-2-(1-methyl-2-oxabicyclo[2.1.1]hexan-4- | |
| | yl)imidazo[1,2-a]pyrimidine-6-carboxylic acid (Preparation 331). | |
| | R-NH₂: 3-amino-1-methylpyridin-2-one | |
| | Prep-HPLC-F | |
| 352 | 7-(Methoxymethyl)-2-(1-methyl-2-oxabicyclo[2.1.1]hexan-4-yl)-N-(6- | 8.0 mg, 21.53% yield |
| | methylpyrazolo[1,5-a]pyrimidin-3-yl)imidazo[1,2-a]pyrimidine-6- | LCMS m/z = 434.3 [M + H]⁺ |
| | carboxamide | ¹H NMR (500 MHz, |
| | | DMSO-d₆) δ: 1.45 (s, 3H), |
| | 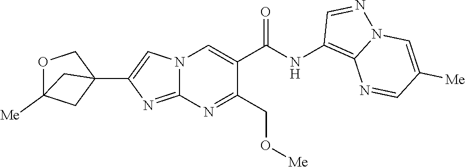 [SMILES: COCc1nc2nc(C34COC(C)(C3)C4)cn2cc1C(=O)Nc1cnn2cc(C)cnc12] | 1.79-1.86 (m, 2H), 2.07 (dd, 2H), 2.31-2.38 (m, 3H), 3.31 (s, 3H) 3.94 (s, 2H), 4.74 (s, 2H), 7.90 (s, 1H), 8.47 (d, 1H), 8.55-8.63 (m, 1H), 8.60 (s, 1H), 8.91-8.98 (m, 1H), 9.27 (s, 1H), 10.82 (s, 1H) |
| | RCO₂H: 7-(methoxymethyl)-2-(1-methyl-2-oxabicyclo[2.1.1]hexan-4- | |
| | yl)imidazo[1,2-a]pyrimidine-6-carboxylic acid (Preparation 331). | |
| | R-NH₂: 6-methylpyrazolo[1,5-a]pyrimidin-3-amine. prep-HPLC-F | |
| 353 | N-(3-cyano-2-fluorophenyl)-7-isopropoxy-2-(1-methyl-2-oxabicyclo[2.1.1] | 3.10 mg, 4.50% yield |
| | hexan-4-yl)imidazo[1,2-a]pyrimidine-6-carboxamide trifluoroacetate | LCMS m/z = 436.3 [M + H]⁺ |
| | | ¹H NMR (500 MHz, |
| | 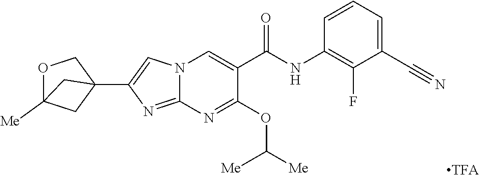 [SMILES: CC(C)Oc1nc2nc(C34COC(C)(C3)C4)cn2cc1C(=O)Nc1cccc(C#N)c1F] | DMSO-d₆) δ: 1.44 (s, 3H), 1.50 (d, 6H), 1.80 (dd, 2H), 2.06 (br d, 2H), 3.91 (s, 2H), 5.52 (spt, 1H), 7.49 (t, 1H), 7.72-7.78 (m, 1H), 7.80 (s, 1H), 8.57 (br s, 1H), 9.49 (s, 1H), 10.27 (s, 1H) |
| | RCO₂H: 7-isopropoxy-2-(1-methyl-2-oxabicyclo[2.1.1]hexan-4- | |
| | yl)imidazo[1,2-a]pyrimidine-6-carboxylic acid (Preparation 128). | |
| | R-NH₂: 3-amino-2-fluorobenzonitrile | |
| | prep-HPLC-D | |
| 354 | 7-Isopropoxy-2-(1-methyl-2-oxabicyclo[2.1.1]hexan-4-yl)-N-(pyridin-2- | 14.3 mg, 28.8% yield |
| | yl)imidazo[1,2-a]pyrimidine-6-carboxamide | LCMS m/z = 394.2 [M + H]⁺ |
| | | ¹H NMR (500 MHz, |
| | 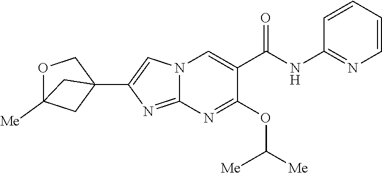 [SMILES: CC(C)Oc1nc2nc(C34COC(C)(C3)C4)cn2cc1C(=O)Nc1ccccn1] | DMSO-d₆) δ: 1.43 (s, 3H), 1.47 (d, 6H), 1.72-1.79 (m, 2H), 1.96-2.03 (m, 2H), 3.88 (s, 2H), 5.41-5.52 (m, 1H), 7.17-7.23 (m, 1H), 7.68 (s, 1H), 7.84-7.92 (m, 1H), 8.23 (br d, 1H), 8.39 (br d, 1H), 9.39 (s, 1H), 10.46 (s, 1H) |
| | RCO₂H: 7-isopropoxy-2-(1-methyl-2-oxabicyclo[2.1.1]hexan-4- | |
| | yl)imidazo[1,2-a]pyrimidine-6-carboxylic acid (Preparation 128). | |
| | R-NH₂: pyridine-2-amine | |
| | prep-HPLC-F | |
| 355 | 7-Isopropoxy-2-(1-methyl-2-oxabicyclo[2.1.1]hexan-4-yl)-N-(6- | 3.5 mg, 3.4% yield |
| | methylpyridin-2-yl)imidazo[1,2-a]pyrimidine-6-carboxamide | LCMS m/z = 408.3 [M + H]⁺ |
| | | ¹H NMR (500 MHz, |
| | 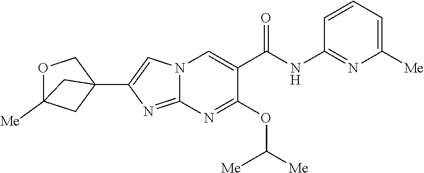 [SMILES: Cc1cccc(NC(=O)c2cn3cc(C45COC(C)(C4)C5)nc3nc2OC(C)C)n1] | DMSO-d₆) δ: 1.43 (s, 3H), 1.47 (br d, 6H), 1.71-1.82 (m, 2H), 1.95-2.07 (m, 2H), 2.43 (s, 3H), 3.88 (s, 2H), 5.36-5.56 (m, 1H), 7.01-7.12 (m, 1H), 7.68 (s, 1H), 7.76 (t, 1H), 8.02 (br d, 1H), 9.36 (s, 1H), 10.47 (s, 1H) |
| | RCO₂H: 7-isopropoxy-2-(1-methyl-2-oxabicyclo[2.1.1]hexan-4- | |
| | yl)imidazo[1,2-a]pyrimidine-6-carboxylic acid (Preparation 128). | |
| | R-NH₂: 6-methylpyridin-2-amine | |
| | prep-HPLC-F | |
| 356 | N-(6-(1,1-difluoroethyl)pyridin-2-yl)-7-isopropoxy-2-(1-methyl-2-oxabicyclo | 36.9 mg, 51.2% yield |
| | [2.1.1]hexan-4-yl)imidazo[1,2-a]pyrimidine-6-carboxamide trifluoroacetate | LCMS m/z = 458.2 [M + H]⁺ |
| | 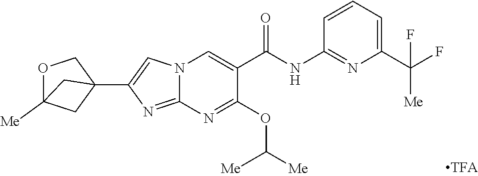 [SMILES: CC(C)Oc1nc2nc(C34COC(C)(C3)C4)cn2cc1C(=O)Nc1cccc(C(C)(F)F)n1] | |
| | RCO₂H: 7-isopropoxy-2-(1-methyl-2-oxabicyclo[2.1.1]hexan-4- | |
| | yl)imidazo[1,2-a]pyrimidine-6-carboxylic acid (Preparation 128). | |
| | R-NH₂: 6-(1,1-difluoroethyl)pyridine-2-amine. prep-HPLC-D | |
| 357 | N-(6-(dimethylamino)pyridin-2-yl)-7-isopropoxy-2-(1-methyl-2-oxabicyclo | 4.10 mg, 5.94% yield |
| | [2.1.1]hexan-4-yl)imidazo[1,2-a]pyrimidine-6-carboxamide trifluoroacetate | LCMS m/z = 437.3 [M + H]⁺ |
| | | ¹H NMR (500 MHz, |
| | 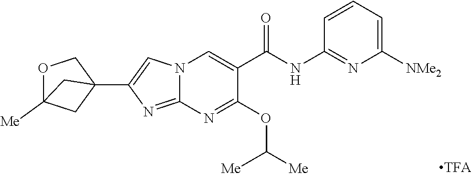 [SMILES: CC(C)Oc1nc2nc(C34COC(C)(C3)C4)cn2cc1C(=O)Nc1cccc(N(C)C)n1] | DMSO-d₆) δ: 1.42-1.46 (m, 3H), 1.53 (br d, 6H), 1.79- 1.83 (m, 2H), 2.02-2.10 (m, 2H), 2.94-3.10 (m, 4H), 3.48 (br d, 2H), 3.91 (s, 2H), 5.45-5.57 (m, 1H), 6.46 (br d, 1H), 7.37-7.45 (m, 1H), 7.58 (t, 1H), 7.82 (s, 1H), 9.50 (br s, 1H), 10.19 (br s, 1H) |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | R-NH₂: N2,N2-dimethylpyridine-2,6-diamine. prep-HPLC-D | |
| 358 | 7-Isopropoxy-N-(1-methyl-2-oxo-1,2-dihydropyridin-3-yl)-2-(1-methyl-2- | 6.10 mg, 5.71% yield |
| | oxabicyclo[2.1.1]hexan-4-yl)imidazo[1,2-a]pyrimidine-6-carboxamide | LCMS m/z = 424.3 [M + H]+ |
| | trifluoroacetate | ¹H NMR (500 MHz, |
| | | DMSO-d₆) δ: 1.44 (s, 3H), |
| | 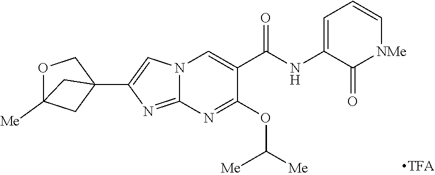 [SMILES: CC(C)Oc1nc2nc(C34COC(C)(C3)C4)cn2cc1C(=O)Nc1cccn(C)c1=O] | 1.56 (d, 6H), 1.73-1.81 (m, 2H), 1.98-2.08 (m, 2H), 3.58 (s, 3H), 3.89 (s, 2H), 5.62 (spt, 1H), 6.35 (t, 1H), 7.50 (dd, 1H), 7.76-7.99 (m, 1H), 8.45 (dd, 1H), 9.53 (s, 1H), 10.69 (s, 1H) |
| | RCO₂H: 7-isopropoxy-2-(1-methyl-2-oxabicyclo[2.1.1]hexan-4- | |
| | yl)imidazo[1,2-a]pyrimidine-6-carboxylic acid (Preparation 128). | |
| | R-NH₂: 3-amino-1-methylpyridin-2-one | |
| | prep-HPLC-D | |
| 359 | 7-Isopropoxy-2-(1-methyl-2-oxabicyclo[2.1.1]hexan-4-yl)-N-(6-(tetrahydro- | 9.20 mg, 12.6% yield |
| | furan-3-yl)pyridin-2-yl)imidazo[1,2-a]pyrimidine-6-carboxamide trifluoroacetate | LCMS m/z = 464.3 [M + H]⁺ |
| | | No nmr available |
| | 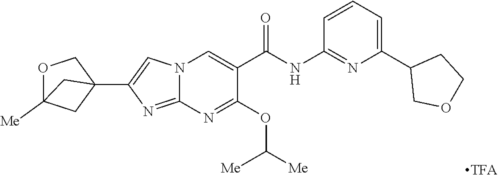 [SMILES: CC(C)Oc1nc2nc(C34COC(C)(C3)C4)cn2cc1C(=O)Nc1cccc(C2CCOC2)n1] | |
| | RCO₂H: 7-isopropoxy-2-(1-methyl-2-oxabicyclo[2.1.1]hexan-4- | |
| | yl)imidazo[1,2-a]pyrimidine-6-carboxylic acid (Preparation 128). | |
| | R-NH₂: 6-tetrahydrofuran-3-ylpyridin-2-amine. prep-HPLC-D | |
| 360 | N-(5-fluoro-1-methyl-1H-pyrazol-3-yl)-7-isopropoxy-2-(1-methyl-2- | 30.2 mg, 36.6% yield |
| | oxabicyclo[2.1.1]hexan-4-yl)imidazo[1,2-a]pyrimidine-6-carboxamide | LCMS m/z = 415.2 [M + H]⁺ |
| | | ¹H NMR (400 MHz, CDCl₃) |
| | 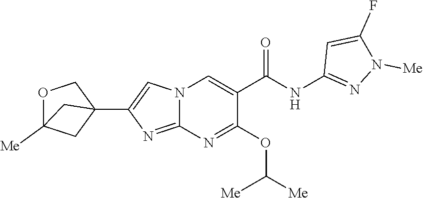 [SMILES: CC(C)Oc1nc2nc(C34COC(C)(C3)C4)cn2cc1C(=O)Nc1cc(F)n(C)n1] | δ: 1.55 (s, 3H), 1.59 (d, 6H), 1.96 (dd, 2H), 2.12 (br d, 2H), 3.70 (d, 3H), 4.08 (s, 2H), 5.79 (spt, 1H), 6.36 (d, 1H), 9.19 (s, 1H), 9.99 (s, 1H) |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | R-NH₂: 5-fluoro-1-methylpyrazol-3-amine hydrochloride. prep-HPLC-F | |
| 361 | N-(1-(difluoromethyl)-1H-pyrazol-3-yl)-7-isopropoxy-2-(1-methyl-2- | 72.1 mg, 44.1% yield |
| | oxabicyclo[2.1.1]hexan-4-yl)imidazo[1,2-a]pyrimidine-6-carboxamide | LCMS m/z = 433.6 [M + H]⁺ |
| | | ¹H NMR (500 MHz, |
| | 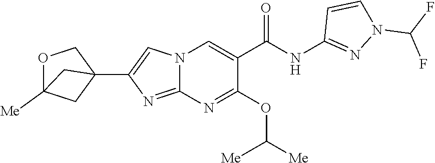 [SMILES: CC(C)Oc1nc2nc(C34COC(C)(C3)C4)cn2cc1C(=O)Nc1ccn(C(F)F)n1] | DMSO-d₆) δ: 1.42-1.45 (m, 9H), 1.75 (dd, 2H), 1.94- 2.03 (m, 2H), 3.87 (s, 2H), 5.35-5.44 (m, 1H), 6.90 (d, 1H), 7.60-7.88 (m, 2H), 8.20 (d, 1H), 9.26 (s, 1H), 10.70 (br s, 1H) |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | R-NH₂: 1-(difluoromethyl)pyrazol-3-amine. prep-HPLC-F | |
| 362 | 7-Isopropoxy-2-(1-methyl-2-oxabicyclo[2.1.1]hexan-4-yl)-N-(1- | 73.3 mg, 43.0% yield |
| | (trifluoromethyl)-1H-pyrazol-3-yl)imidazo[1,2-a]pyrimidine-6-carboxamide | LCMS m/z = 451.5 [M + H]⁺ |
| | | ¹H NMR (500 MHz, |
| | 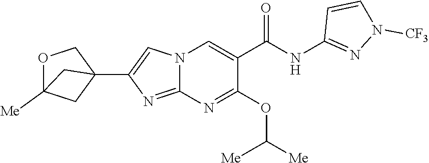 [SMILES: CC(C)Oc1nc2nc(C34COC(C)(C3)C4)cn2cc1C(=O)Nc1ccn(C(F)(F)F)n1] | DMSO-d₆) δ ppm 1.38-1.46 (m, 9H), 1.75 (dd, 2H), 1.98-2.04 (m, 2H), 3.87 (s, 2H), 5.38 (spt, 1H), 7.01 (br s, 1H), 7.64 (s, 1H), 8.47 (d, 1H), 9.25 (s, 1H), 10.91 (br s, 1H) |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | R-NH₂: 1-(trifluoromethyl)pyrazol-3-amine. prep-HPLC-F | |
| 363 | N-(1-cyclopropyl-1H-pyrazol-3-yl)-7-isopropoxy-2-(1-methyl-2-oxabicyclo | 7.50 mg, 9.4% yield |
| | [2.1.1]hexan-4-yl)imidazo[1,2-a]pyrimidine-6-carboxamide trifluoroacetate | LCMS m/z = 423.3 [M + H]⁺ |
| | | ¹H NMR (500 MHz, |
| | 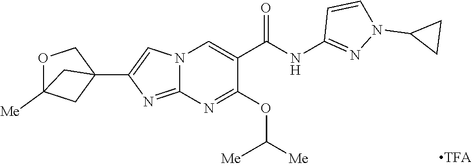 [SMILES: CC(C)Oc1nc2nc(C34COC(C)(C3)C4)cn2cc1C(=O)Nc1ccn(C2CC2)n1] | DMSO-d₆) δ: 0.91-1.04 (m, 4H), 1.44 (d, 6H), 1.46 (s, 3H), 1.77-1.85 (m, 2H), 2.04-2.12 (m, 2H), 3.67 (tt, 1H), 3.91 (s, 2H), 5.37-5.47 (m, 1H), 6.58 (d, 1H), 7.75 (d, 1H), 7.80 (br s, 1H), 9.32 (s, 1H), 10.57 (br s, 1H) |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | R-NH₂: 1-cyclopropylpyrazol-3-amine | |
| | prep-HPLC-D | |
| 364 | N-(1-cyclobutyl-1H-pyrazol-3-yl)-7-isopropoxy-2-(1-methyl-2-oxabicyclo | 5.0 mg, 6.1% yield |
| | [2.1.1]hexan-4-yl)imidazo[1,2-a]pyrimidine-6-carboxamide trifluoroacetate | LCMS m/z = 437.3 [M + H]⁺ |
| | | ¹H NMR (500 MHz, |
| | 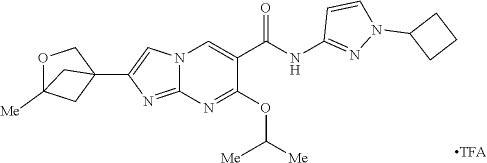 [SMILES: CC(C)Oc1nc2nc(C34COC(C)(C3)C4)cn2cc1C(=O)Nc1ccn(C2CCC2)n1] | DMSO-d₆) δ: 1.44 (d, 6H), 1.46 (d, 3H), 1.73-1.83 (m, 2H), 1.79-1.83 (m, 2H), 2.07-2.12 (m, 2H), 2.33-2.40 (m, 2H), 2.40-2.47 (m, 2H), 3.91 (s, 2H), 4.78 (quin, 1H), 5.42 (spt, 1H), 6.60 (d, 1H), 7.77 (d, 1H), 7.82 (s, 1H), 9.32 (s, 1H), 10.70 (br s, 1H) |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | R-NH₂: 1-cyclobutylpyrazol-3-amine | |
| | prep-HPLC-D | |
| 365 | 7-Isopropoxy-N-(2-methyl-2H-1,2,3-triazol-4-yl)-2-(1-methyl-2-oxabicyclo | 47.4 mg, 49.0% yield |
| | [2.1.1]hexan-4-yl)imidazo[1,2-a]pyrimidine-6-carboxamide trifluoroacetate | LCMS m/z = 398.2 [M + H]⁺ |
| | | ¹H NMR (500 MHz, |
| | 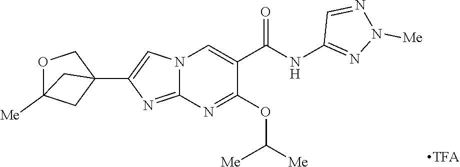 [SMILES: CC(C)Oc1nc2nc(C34COC(C)(C3)C4)cn2cc1C(=O)Nc1cnn(C)n1] | DMSO-d₆) δ: 1.39-1.48 (m, 9H), 1.75 (dd, 2H), 2.00 (dd, 2H), 3.88 (s, 2H), 4.11 (s, 3H), 5.41 (spt, 1H), 7.64 (s, 1H), 7.97 (s, 1H), 9.27 (s, 1H), 10.68 (s, 1H) |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | R-NH₂: 2-methyltriazol-4-amine | |
| | prep-HPLC-D | |
| 366 | N-(6,7-dihydro-5H-cyclopenta[b]pyridin-2-yl)-7-isopropoxy-2-(1-methyl-2- | 19.2 mg, 22.2% yield |
| | oxabicyclo[2.1.1]hexan-4-yl)imidazo[1,2-a]pyrimidine-6-carboxamide | LCMS m/z = 434.3 [M + H]⁺ |
| | trifluoroacetate | ¹H NMR (500 MHz, |
| | | DMSO-d₆) δ: 1.42-1.50 (m, |
| | 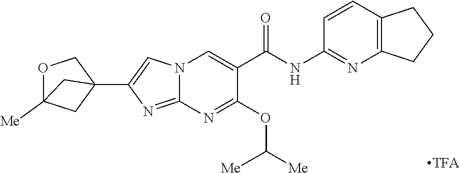 [SMILES: CC(C)Oc1nc2nc(C34COC(C)(C3)C4)cn2cc1C(=O)Nc1ccc2c(n1)CCC2] | 9H), 1.76-1.82 (m, 2H), 2.03-2.10 (m, 4H), 2.86-2.90 (m, 4H), 3.85-3.92 (m, 2H), 5.42-5.51 (m, 1H), 7.69 (d, 1H), 7.77 (br s, 1H), 7.99 (br d, 1H), 9.39 (s, 1H), 10.50 (br s, 1H) |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | R-NH₂: 6,7-dihydro-5H-cyclopenta[b]pyridine-2-amine. prep-HPLC-D | |
| 367 | N-(4-(difluoromethyl)thiazol-2-yl)-7-isopropoxy-2-(1-methyl-2- | 10.2 mg, 14.4% yield as a |
| | oxabicyclo[2.1.1]hexan-4-yl)imidazo[1,2-a]pyrimidine-6-carboxamide | white solid |
| | | LCMS m/z = 450.1 [M + H]⁺ |
| | 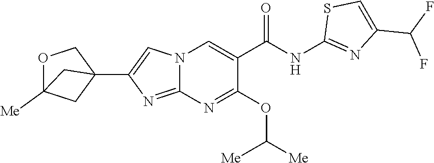 [SMILES: CC(C)Oc1nc2nc(C34COC(C)(C3)C4)cn2cc1C(=O)Nc1nc(C(F)F)cs1] | ¹H NMR (500 MHz, MeOH- d₄) δ: 1.50 (s, 3H), 1.59 (d, 6H), 1.80-1.90 (m, 2H), 2.00-2.10 (m, 2H), 4.01 (s, 2H), 5.70-5.80 (m, 1H), 6.60-6.90 (m, 1H), 7.54 (s, 1H), 7.62 (s, 1H), 9.38 (s, 1H) |
| | RCO₂H: | |
| | R-NH₂: 4-(difluoromethyl)-1,3-thiazol-2-amine. prep-HPLC-J | |
| 368 | 7-Isopropoxy-2-(1-methyl-2-oxabicyclo[2.1.1]hexan-4-yl)-N-(6-(oxazol-5- | 31.1 mg, 42.8% yield as an |
| | yl)pyridin-2-yl)imidazo[1,2-a]pyrimidine-6-carboxamide | off-white solid |
| | | LCMS m/z = 461.1 [M + H]⁺ |
| | 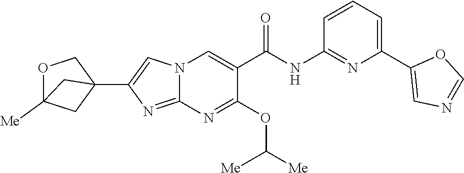 [SMILES: CC(C)Oc1nc2nc(C34COC(C)(C3)C4)cn2cc1C(=O)Nc1cccc(-c2cnco2)n1] | ¹H NMR (500 MHz, MeOH- d₄) δ: 1.50 (s, 3H), 1.65 (d, 6H), 1.84-1.90 (m, 2H), 2.09-2.14 (m, 2H), 4.01 (s, 2H), 5.60-5.70 (m, 1H), 7.56 (d, 1H), 7.61-7.64 (m, 2H), 7.94 (t, 1H), 8.28 (d, 1H), 8.34 (s, 1H), 9.38 (s, 1H) |
| | RCO₂H: | |
| | RNH₂: 6-(oxazol-5-yl)pyridin-2-amine trifluoroacetate (Preparation X). | |
| | prep-HPLC-J | |
| 369 | N-(6-(difluoromethyl)pyrazolo[1,5-a]pyrimidin-3-yl)-7-isopropoxy-2-(1-methyl- | 15.6 mg, 20.5% yield as a |
| | 2-oxabicyclo[2.1.1]hexan-4-yl)imidazo[1,2-a]pyrimidine-6-carboxamide | yellow solid |
| | | LCMS m/z = 484.1 [M + H]⁺ |
| | 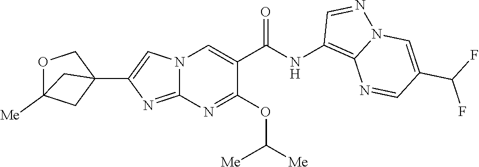 [SMILES: CC(C)Oc1nc2nc(C34COC(C)(C3)C4)cn2cc1C(=O)Nc1cnn2cc(C(F)F)cnc12] | ¹H NMR (500 MHz, CDCl₃) δ: 1.54 (s, 3H), 1.65 (d, 6H), 1.80-1.90 (m, 2H), 2.00-2.10 (m, 2H), 4.09 (s, 2H), 5.80- 5.90 (m, 1H), 6.80-7.00 (m, 1H), 7.30 (s, 1H), 8.56 (s, 1H), 8.78 (s, 1H), 9.05 (s, 1H), 9.24 (s, 1H), 10.49 (s, 1H) |
| | RCO₂H: | |
| | R-NH₂: 6-(difluoromethyl)pyrazolo[1,5-a]pyrimidin-3-amine (Preparation X) | |
| | prep-HPLC-J | |
| 370 | 7-(Cyclopropylmethoxy)-N-(1-methyl-1H-pyrazol-3-yl)-2-(1-methyl-2- | 35.5 mg, 50.9% yield as a |
| | oxabicyclo[2.1.1]hexan-4-yl)imidazo[1,2-a]pyrimidine-6-carboxamide | white solid |
| | | LCMS /z = 460.1 [M + H]⁺ |
| | 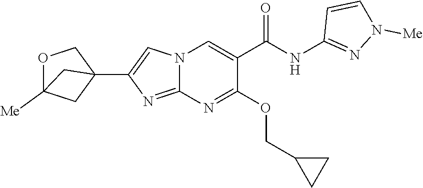 [SMILES: Cn1ccc(NC(=O)c2cn3cc(C45COC(C)(C4)C5)nc3nc2OCC2CC2)n1] | ¹H NMR (400 MHz, MeOH- d₄) δ: 0.50-0.57 (m, 2H), 0.70-0.82 (m, 2H), 1.48 (s, 3H), 1.52-1.60 (m, 1H), 1.80-1.90 (m, 2H), 2.06-2.13 (m, 2H), 2.40 (s, 3H), 3.99 (s, 2H), 4.55 (d, 2H), 7.64 (s, 1H), 8.42 (s, 1H), 8.65 (s, 1H), 8.69 (s, 1H), 9.38 (s, 1H) |
| | RCO₂H: 7-(cyclopropylmethoxy)-2-(1-methyl-2-oxabicyclo[2.1.1]hexan-4- | |
| | yl)imidazo[1,2-a]pyrimidine-6-carboxylic acid (Preparation 314). | |
| | RNH₂: 1-methyl-1H-pyrazol-3-amine | |
| | prep-HPLC-J | |
| 371 | 7-Cyclobutoxy-2-(1-methyl-2-oxabicyclo[2.1.1]hexan-4-yl)-N-(pyrazolo[1,5- | 43.1 mg, 63.7% as a green |
| | a]pyrimidin-3-yl)imidazo[1,2-a]pyrimidine-6-carboxamide | solid |
| | | LCMS m/z = 446.1 [M + H]⁺ |
| | 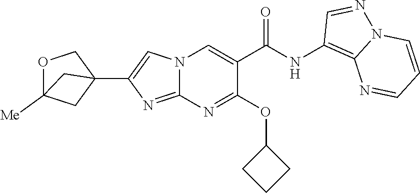 [SMILES: CC12CC(c3cn4cc(C(=O)Nc5cnn6cccnc56)c(OC5CCC5)nc4n3)(CO1)C2] | ¹H NMR (500 MHz, CDCl₃) δ: 1.54 (s, 3H), 1.77-1.87 (m, 1H), 1.96 (dd, 2H), 1.99-2.06 (m, 1H), 2.08-2.13 (m, 2H), 2.49-2.59 (m, 2H), 2.69-2.78 (m, 2H), 4.08 (s, 2H), 5.65 (q, 1H), 6.85 (dd, 1H), 7.30 (s, 1H), 8.45 (d, 1H), 8.64 (dd, 1H), 8.93 (s, 1H), 9.24 (s, 1H), 10.44 (s, 1H) |
| | RCO₂H: 7-cyclobutoxy-2-(1-methyl-2-oxabicyclo[2.1.1]hexan-4-yl)imidazo[1,2- | |
| | a]pyrimidine-6-carboxylic acid (Preparation313). | |
| | RNH₂: pyrazolo[1,5-a]pyrimidin-3-amine. prep-HPLC-J | |
| 372 | 7-Isopropoxy-2-(1-methyl-2-oxabicyclo[2.1.1]hexan-4-yl)-N-(pyrazolo[1,5- | 22.10 mg, 27% yield |
| | a]pyrimidin-5-yl)imidazo[1,2-a]pyrimidine-6-carboxamide | LCMS m/z = 434.1 [M + H]⁺ |
| | | ¹H NMR (500 MHz, |
| | 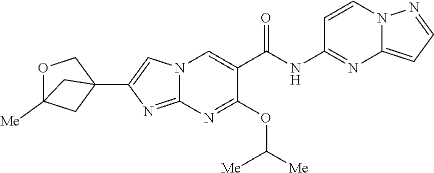 [SMILES: CC(C)Oc1nc2nc(C34COC(C)(C3)C4)cn2cc1C(=O)Nc1ccn2nccc2n1] | DMSO-d₆) δ: 1.43-1.50 (m, 9H), 1.73-1.81 (m, 2H), 1.97-2.05 (m, 2H), 3.86-3.92 (m, 2H), 5.38-5.48 (m, 1H), 6.50 (d, 1H), 7.68 (s, 1H), 7.87 (d, 1H), 8.17 (d, 1H), 9.13 (d, 1H), 9.38 (s, 1H), 10.84 (s, 1H) |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | RNH₂: pyrazolo[1,5-a]pyrimidin-5-amine. prep-HPLC-F | |
| 373 | N-(imidazo[1,2-b]pyridazin-3-yl)-7-isopropoxy-2-(1-methyl-2- | 75 mg, 54.9% yield |
| | oxabicyclo[2.1.1]hexan-4-yl)imidazo[1,2-a]pyrimidine-6-carboxamide | LCMS m/z = 434.2 [M + H]⁺ |
| | | ¹H NMR (400 MHz, CDCl₃) |
| | 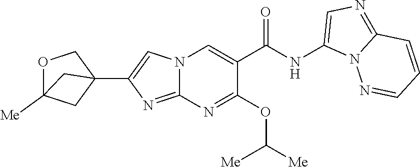 [SMILES: CC(C)Oc1nc2nc(C34COC(C)(C3)C4)cn2cc1C(=O)Nc1cnc2cccnn12] | δ: 1.56 (s, 3H), 1.68 (d, 6H), 1.95-2.03 (m, 2H), 2.09-2.15 (m, 2H), 4.10 (s, 2H), 5.92 (spt, 1H), 7.06 (dd, 1H), 7.33 (s, 1H), 7.96-8.06 (m, 1H), 8.35 (s, 1H), 8.40 (d, 1H), 9.28 (s, 1H), 11.03 (s, 1H) |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | RNH₂: imidazo[1,2-b]pyridazine-3-amine. prep-HPLC-F | |
| 374 | 7-Isopropoxy-2-(1-methyl-2-oxabicyclo[2.1.1]hexan-4-yl)-N-(5-methyl- | 11.5 mg, 10.2% |
| | pyrazolo[1,5-a]pyrimidin-3-yl)imidazo[1,2-a]pyrimidine-6-carboxamide | LCMS m/z = 448.2 [M + H]⁺ |
| | | ¹H NMR (500 MHz, |
| | 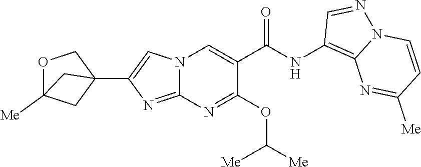 [SMILES: Cc1ccn2ncc(NC(=O)c3cn4cc(C56COC(C)(C5)C6)nc4nc3OC(C)C)c2n1] | DMSO-d₆) δ: 1.44 (s, 3H), 1.60 (d, 6H), 1.77 (dd, 2H), 1.95-2.05 (m, 2H), 2.58 (s, 3H), 3.89 (s, 2H), 5.58 (spt, 1H), 6.95-6.98 (m, 1H), 7.74 (s, 1H), 8.64-8.69 (m, 1H), 8.96 (d, 1H), 9.48 (s, 1H), 10.31 (s, 1H) |
| | RCO₂H: 7-isopropoxy-2-(1-methyl-2-oxabicyclo[2.1.1]hexan-4- | |
| | yl)imidazo[1,2-a]pyrimidine-6-carboxylic acid (Preparation 128).. | |
| | RNH₂: 5-methylpyrazolo[1,5-a]pyrimidin-3-amine. prep-HPLC-F | |
| 375 | N-(6-cyclopropylpyrazolo[1,5-a]pyrimidin-3-yl)-7-isopropoxy-2-(1-methyl-2- | 5.5 mg, 9.2% yield |
| | oxabicyclo[2.1.1]hexan-4-yl)imidazo[1,2-a]pyrimidine-6-carboxamide | LCMS m/z = 474.2 [M + H]⁺ |
| | | ¹H NMR (500 MHz, |
| | 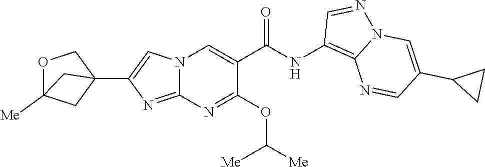 [SMILES: CC(C)Oc1nc2nc(C34COC(C)(C3)C4)cn2cc1C(=O)Nc1cnn2cc(C3CC3)cnc12] | DMSO-d₆) δ: 0.86-0.95 (m, 2H), 0.97-1.06 (m, 2H), 1.44 (s, 3H), 1.56 (d, 6H), 1.76 (dd, 2H), 2.01 (dd, 2H), 2.02-2.10 (m, 1H), 3.89 (s, 2H), 5.55 (spt, 1H), 7.72 (s, 1H), 8.44-8.91 (m, 2H), 9.45 (s, 1H) 10.31 (s, 1H) |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation1 28). | |
| | RNH₂: 6-cyclopropylpyrazolo[1,5-a]pyrimidin-3-amine dihydrochloride | |
| | prep-HPLC-F | |
| 376 | 7-Isopropoxy-N-(isothiazolo[4,3-b]pyridin-3-yl)-2-(1-methyl-2-oxabicyclo | 2.2 mg, 1.94% yield |
| | [2.1.1]hexan-4-yl)imidazo[1,2-a]pyrimidine-6-carboxamide | LCMS m/z = 451.1 [M + H]+ |
| | | ¹H NMR (500 MHz, |
| | 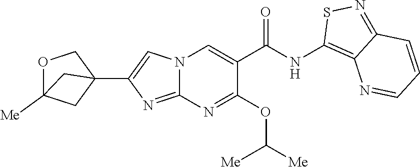 [SMILES: CC(C)Oc1nc2nc(C34COC(C)(C3)C4)cn2cc1C(=O)Nc1snc2cccnc12] | DMSO-d₆) δ: 1.44 (s, 3H), 1.60 (d, 6H), 1.78 (dd, 2H), 1.99-2.05 (m, 2H), 3.90 (s, 2H), 5.56-5.61 (m, 1H), 7.51-7.61 (m, 1H), 7.75 (s, 1H), 8.14 (dd, 1H), 8.74 (dd, 1H), 9.60 (s, 1H) |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | RNH₂: isothiazolo[4,3-b]pyridine-3-amine. prep-HPLC-F | |
| 377 | N-(2,3-dihydrobenzofuran-7-yl)-7-isopropoxy-2-(1-methyl-2-oxabicyclo | 5.3 mg, 6.1% yield |
| | [2.1.1]hexan-4-yl)imidazo[1,2-a]pyrimidine-6-carboxamide trifluoroacetate | LCMS m/z = 435.3 [M + H]⁺ |
| | | ¹H NMR (500 MHz, |
| | 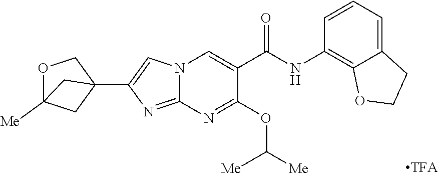 [SMILES: CC(C)Oc1nc2nc(C34COC(C)(C3)C4)cn2cc1C(=O)Nc1cccc2c1OCC2] | DMSO-d₆) δ: 1.44 (s, 3H), 1.52 (d, 6H), 1.76-1.86 (m, 2H), 2.01-2.11 (m, 2H), 3.29 (t, 1H), 3.48 (br d, 1H), 3.90 (s, 2H), 4.69 (t, 2H), 5.53- 5.66 (m, 1H), 6.88 (t, 1H), 7.01-7.09 (m, 1H), 7.82 (s, 1H), 8.09 (d, 1H), 9.53 (s, 1H), 9.94 (s, 1H) |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | RNH₂: 2,3-dihydrobenzofuran-7-amine | |
| | prep-HPLC-D | |
| 378 | N-(benzo[d]thiazol-4-yl)-7-isopropoxy-2-(1-methyl-2-oxabicyclo[2.1.1]hexan- | 3.4 mg, 6% yield |
| | 4-yl)imidazo[1,2-a]pyrimidine-6-carboxamide | LCMS m/z = 450.3 [M + H]⁺ |
| | | ¹H NMR (500 MHz, |
| | 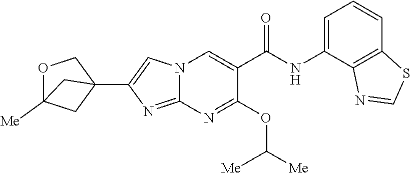 [SMILES: CC(C)Oc1nc2nc(C34COC(C)(C3)C4)cn2cc1C(=O)Nc1cccc2scnc12] | DMSO-d₆) δ: 1.44 (s, 3H), 1.62 (d, 6H), 1.77 (dd, 2H), 1.98-2.06 (m, 2H), 3.89 (s, 2H), 5.71 (spt, 1H), 7.54 (t, 1H), 7.77 (s, 1H), 7.89-8.00 (m, 1H), 8.61 (d, 1H), 9.50 (s, 1H), 9.58 (s, 1H), 11.23 (s, 1H) |
| | RCO₂H: 7-isopropoxy-2-(1-methyl-2-oxabicyclo[2.1.1]hexan-4- | |
| | yl)imidazo[1,2-a]pyrimidine-6-carboxylic acid (Preparation 128). | |
| | RNH₂: 1,3-benzothiazol-4-amine | |
| | prep-HPLC-F | |
| 379 | 7-Isopropoxy-N-(1-methyl-1H-benzo[d]imidazol-4-yl)-2-(1-methyl-2- | 48.6 mg, 45.9% yield |
| | oxabicyclo[2.1.1]hexan-4-yl)imidazo[1,2-a]pyrimidine-6-carboxamide | LCMS m/z = 447.2 [M + H]⁺ |
| | trifluoroacetate | ¹H NMR (500 MHz, |
| | | DMSO-d₆) δ: 1.44 (s, 3H), |
| | 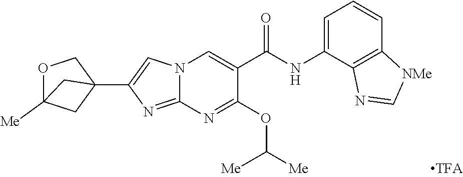 [SMILES: CC(C)Oc1nc2nc(C34COC(C)(C3)C4)cn2cc1C(=O)Nc1cccc2c1ncn2C] | 1.64 (d, 6H), 1.74-1.82 (m, 2H), 2.02 (dd, 2H), 3.89 (d, 5 H), 5.69 (spt, 1H), 7.26- 7.33 (m, 1H), 7.35-7.40 (m, 1H), 7.77 (s, 1H), 8.27 (t, 2H), 9.55 (s, 1H), 10.86 (s, 1H) |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | RNH₂: 1-methylbenzimidazol-4-amine | |
| | prep-HPLC-D | |
| 380 | 7-Isopropoxy-2-(1-methyl-2-oxabicyclo[2.1.1]hexan-4-yl)-N-(thieno[2,3- | 10.9 mg, 10.2% yield |
| | b]pyrazin-7-yl)imidazo[1,2-a]pyrimidine-6-carboxamide trifluoroacetate | LCMS m/z = 451.2 [M + H]⁺ |
| | | ¹H NMR (500 MHz, |
| | 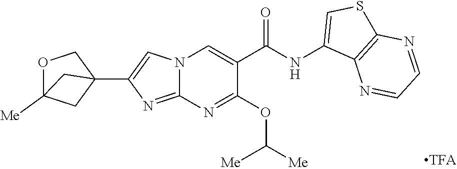 [SMILES: CC(C)Oc1nc2nc(C34COC(C)(C3)C4)cn2cc1C(=O)Nc1csc2nccnc12] | DMSO-d₆) δ: 1.44 (s, 3H), 1.62 (d, 6H), 1.78 (dd, 2H), 2.00-2.06 (m, 2H), 3.90 (s, 2H), 5.65 (spt, 1H), 7.78 (s, 1H), 8.51-8.65 (m, 1H), 8.77-8.92 (m, 2H), 9.58 (s, 1H), 11.00 (s, 1H) |
| | RCO₂H: 7-isopropoxy-2-(1methyl-2-oxabicyclo[2.1.1]hexan-4- | |
| | yl)imidazo[1,2-a]pyrimidine-6-carboxylic acid (Preparation 128). | |
| | RNH₂: thieno[2,3-b]pyrazin-7-amine | |
| | prep-HPLC-D | |
| 381 | N-(1-(difluoromethyl)-2-oxo-1,2-dihydropyridin-3-yl)-2-(1-(fluoromethyl)-2- | 51.6 mg, 48.3% yield |
| | oxabicyclo[2.1.1]hexan-4-yl)-7-isopropoxyimidazo[1,2-a]pyrimidine-6- | LCMS m/z = 478.2 [M + H]⁺ |
| | carboxamide | 1H NMR (500 MHz, |
| | | DMSO-d₆) δ: 1.54 (d, 6H), |
| | 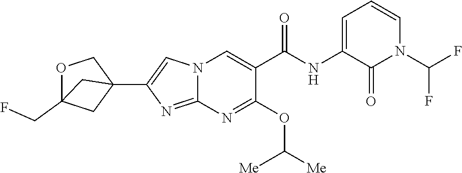 [SMILES: CC(C)Oc1nc2nc(C34COC(CF)(C3)C4)cn2cc1C(=O)Nc1cccn(C(F)F)c1=O] | 1.83-1.90 (m, 2H), 2.10-2.21 (m, 2H), 3.96 (s, 2H), 4.65- 4.77 (m, 2H), 5.57-5.67 (m, 1H), 6.57 (t, 1H), 7.61 (dd, 1H), 7.78 (s, 1H), 7.87-8.18 (m, 1H), 8.54 (dd, 1H), 9.52 (s, 1H), 10.63 (s, 1H) |
| | RCO₂H: 2-(1-(fluoromethyl)-2-oxabicyclo[2.1.1]hexan-4-yl)-7-isopropoxy- | |
| | imidazo[1,2-a]pyrimidine-6-carboxylic acid (Preparation 311) | |
| | RNH₂: 3-amino-1-(difluoromethyl)pyridine-2-one. prep-HPLC-F | |
| 382 | N-(5-fluoro-1-methyl-1H-pyrazol-3-yl)-7-isopropoxy-2-(1-methyl-2- | 26.6 mg, 21% yield |
| | oxabicyclo[2.2.2]octan-4-yl)imidazo[1,2-a]pyrimidine-6-carboxamide | LCMS m/z = 443.2 [M + H]⁺ |
| | | ¹H NMR (400 MHz, CDCl₃) |
| | 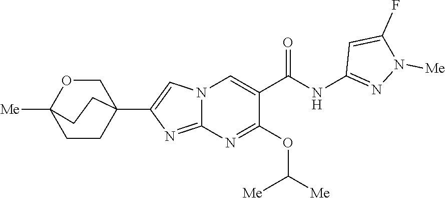 [SMILES: CC(C)Oc1nc2nc(C34CCC(C)(CC3)OC4)cn2cc1C(=O)Nc1cc(F)n(C)n1] | δ: 1.19 (s, 3H), 1.58 (d, 6H), 1.71-1.81 (m, 2H), 1.94-2.07 (m, 4H), 2.17-2.30 (m, 2H) 3.70 (s, 3H), 4.13 (s, 2H), 5.74-5.89 (m, 1H), 6.36 (d, 1H), 7.17 (s, 1H), 9.17 (d, 1H), 10.00 (s, 1H) |
| | RCO₂H: 7-isopropoxy-2-(1-methyl-2-oxabicyclo[2.2.2]octan-4- | |
| | yl)imidazo[1,2-a]pyrimidine-6-carboxylic acid (Preparation 359). | |
| | RNH₂: 5-fluoro-1-methylpyrazol-3-amine. prep-HPLC-F | |
| 383 | N-(6-(difluoromethyl)pyridin-2-yl)-7-isopropoxy-2-(1-methyl-2- | 47.8 mg, 35.3% yield |
| | oxabicyclo[2.2.2]octan-4-yl)imidazo[1,2-a]pyrimidine-6-carboxamide | LCMS m/z = 472.2 [M + H]⁺ |
| | | ¹H NMR (400 MHz, CDCl₃) |
| | 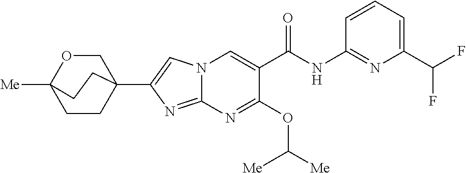 [SMILES: CC(C)Oc1nc2nc(C34CCC(C)(CC3)OC4)cn2cc1C(=O)Nc1cccc(C(F)F)n1] | δ: 1.19 (s, 3H), 1.61 (d, 6H), 1.72-1.81 (m, 2H), 1.93-2.05 (m, 4H), 2.19-2.30 (m, 2H) 4.13 (s, 2H), 5.81 (spt, 1H), 6.37-6.72 (m, 1H), 7.20 (s, 1H), 7.44 (d, 1H), 7.91 (t, 1H), 8.45 (dd, 1H), 9.20 (s, 1H), 10.56 (s, 1H) |
| | RCO₂H: 7-isopropoxy-2-(1-methyl-2-oxabicyclo[2.2.2]octan-4-yl)imidazo[1,2- | |
| | a]pyrimidine-6-carboxylic acid (Preparation 359). | |
| | RNH₂: 6-(difluoromethyl)pyridine-2-amine hydrochloride. prep-HPLC-F | |
| 384 | N-(1-(difluoromethyl)-2-oxo-1,2-dihydropyridin-3-yl)-7-isopropoxy-2-(1- | 66.40 mg, 47.52% yield |
| | methyl-2-oxabicyclo[2.2.2]octan-4-yl)imidazo[1,2-a]pyrimidine-6-carboxamide | LCMS m/z = 488.2 [M + H]⁺ |
| | | ¹H NMR (400 MHz, CDCl₃) |
| | 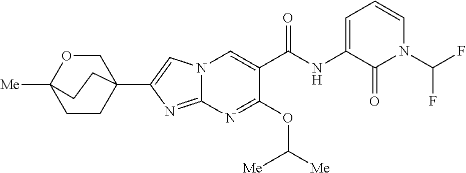 [SMILES: CC(C)Oc1nc2nc(C34CCC(C)(CC3)OC4)cn2cc1C(=O)Nc1cccn(C(F)F)c1=O] | δ: 1.10 (s, 3H), 1.51 (d, 6H), 1.62-1.72 (m, 2H), 1.84-1.96 (m, 4H), 2.08-2.19 (m, 2H), 4.04 (s, 2H), 5.80 (spt, 1H), 6.32 (t, 1H), 7.08 (s, 1H), 7.17 (dd, 1H), 7.52-7.91 (m, 1H), 8.51 (dd, 1H), 9.06 (s, 1H), 10.70 (s, 1H) |
| | RCO₂H: 7-isopropoxy-2-(1-methyl-2-oxabicyclo[2.2.2]octan-4-yl)imidazo[1,2- | |
| | a]pyrimidine-6-carboxylic acid (Preparation 359). | |
| | RNH₂: 3-amino-1-(difluoromethyl)pyridine-2-one. prep-HPLC-F | |
| 385 | 7-Isopropoxy-2-(1-methyl-2-oxabicyclo[2.1.1]hexan-4-yl)-N- | 2.1 mg, 3% |
| | (pyrazolo[1,5-a]pyridin-7-yl)imidazo[1,2-a]pyrimidine-6-carboxamide | LCMS m/z = 443.0 [M + H]⁺ |
| | | ¹H NMR (500 MHz, |
| | 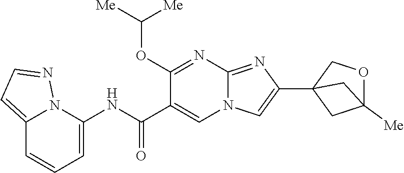 [SMILES: CC(C)Oc1nc2nc(C34COC(C)(C3)C4)cn2cc1C(=O)Nc1cccc2ccnn12] | DMSO-d₆) δ: 1.44 (s, 3H), 1.64 (d, 6H), 1.76-1.84 (m, 2H), 2.03 (dd, 2H), 3.90 (s, 2H), 5.75 (quin, 1H), 6.76 (d, 1H), 7.36 (dd, 1H), 7.54 (dd, 1H), 7.78 (s, 1H), 7.86 (d, 1H) 8.17 (d, 1H), 9.63 (s, 1H), 11.71 (s, 1H). |
| | RCO₂H: 7-isopropoxy-2-(1-methyl-2-oxabicyclo[2.1.1]hexan-4- | |
| | yl)imidazo[1,2-a]pyrimidine-6-carboxylic acid (Preparation 128). | |
| | RNH₂: pyrazolo[1,5-a]pyridin-7-amine | |
| | prep-HPLC-F | |
| 386 | N-(3-(difluoromethyl)phenyl)-7-isopropoxy-2-(1-methyl-2-oxabicyclo[2.1.1] | 44.8 mg, 54 % |
| | hexan-4-yl)imidazo[1,2-a]pyrimidine-6-carboxamide | LCMS m/z = 443.0 [M + H]⁺ |
| | | ¹H NMR (500 MHz, |
| | 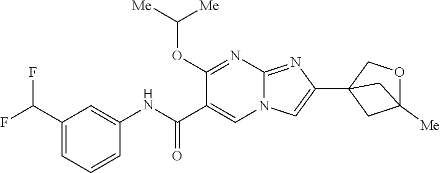 [SMILES: CC(C)Oc1nc2nc(C34COC(C)(C3)C4)cn2cc1C(=O)Nc1cccc(C(F)F)c1] | DMSO-d₆) δ: 1.39-1.51 (m, 10H), 1.80 (dd, 2H), 2.06 (br d, 2H), 3.91 (s, 2H), 5.34- 5.48 (m, 1H), 6.91-7.25 (m, 1H), 7.36 (d, 1H), 7.55 (t, 1H), 7.75 (br d, 2H), 8.03 (s, 1H), 9.32 (s, 1H), 10.40 (br s, 1H). |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | RNH₂: 3-(difluoromethyl)aniline | |
| | prep-HPLC-F | |
| 387 | N-(6-cyclopropylpyridin-2-yl)-7-isopropoxy-2-(1-methyl-2- | 4.0 mg, 5% |
| | oxabicyclo[2.1.1]hexan-4-yl)imidazo[1,2-a]pyrimidine-6-carboxamide | LCMS m/z = 434 [M + H]⁺ |
| | | ¹H NMR (500 MHz, |
| | 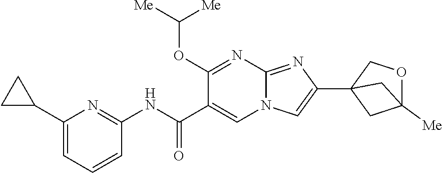 [SMILES: CC(C)Oc1nc2nc(C34COC(C)(C3)C4)cn2cc1C(=O)Nc1cccc(C2CC2)n1] | DMSO-d₆) δ: 0.86-1.01 (m, 4H), 1.44 (s, 3H), 1.51 (br d, 5H), 1.79 (dd, 2H), 1.96- 2.17 (m, 3H), 5.40-5.59 (m, 1H), 7.15 (d, 1H), 7.65-7.79 (m, 1H), 7.94 (br d, 1H), 9.46 (s, 1H), 10.41 (br s, 1H). |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | RNH₂: 6-Cyclopropylpyridin-2-amine | |
| | prep-HPLC-F | |
| 388 | N-(1-(difluoromethyl)-2-oxo-1,2-dihydropyridin-3-yl)-7-isopropoxy-2-(1-methyl- | 43 mg, 60% |
| | 2-oxabicyclo[2.1.1]hexan-4-yl)imidazo[1,2-a]pyrimidine-6-carboxamide | LCMS m/z = 460 [M + H]⁺ |
| | | ¹H NMR (500 MHz, |
| | 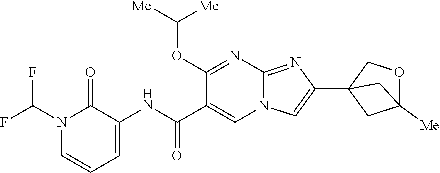 [SMILES: CC(C)Oc1nc2nc(C34COC(C)(C3)C4)cn2cc1C(=O)Nc1cccn(C(F)F)c1=O] | DMSO-d₆) δ: 1.44 (s, 3H), 1.54 (d, 6H), 1.77 (dd, 2H), 2.01 (dd, 2H), 3.88 (s, 2H), 5.62 (t, IH), 6.57 (t, 1H), 7.61 (dd, 1H), 7.75 (s, 1H), 7.86- 8.23 (m, 1H), 8.53 (dd, 1H), 9.52 (s, 1H), 10.62 (s, 1H). |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | RNH₂: 3-amino-1-(difluoromethyl)pyridin-2-one. prep-HPLC-F | |
| 389 | N-(2-(difluoromethoxy)pyridin-3-yl)-7-isopropoxy-2-(1-methyl-2- | 16.2 mg, 22% |
| | oxabicyclo[2.1.1]hexan-4-yl)imidazo[1,2-a]pyrimidine-6-carboxamide | LCMS m/z = 460 [M + H]⁺ |
| | | ¹H NMR (500 MHz, |
| | 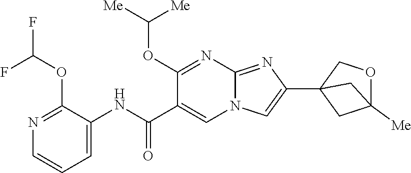 [SMILES: CC(C)Oc1nc2nc(C34COC(C)(C3)C4)cn2cc1C(=O)Nc1cccnc1OC(F)F] | DMSO-d₆) δ: 1.44 (s, 3H), 1.51 (d, 6H), 1.76 (dd, 2H), 2.01 (dd, 2H), 3.88 (s, 2H), 5.57-5.77 (m, 1H), 7.37 (dd, 1H), 7.61-8.28 (m, 1H), 7.99- 8.12 (m, 1H), 8.83 (dd, 1H), 9.51 (s, 1H), 9.99 (s, 1H). |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | RNH₂: 2-(difluoromethoxy)pyridin-3-amine. prep-HPLC-F | |
| 390 | N-(6-(difluoromethyl)pyridin-2-yl)-7-ethoxy-2-(1-methoxy- | 30 mg, 5.1% yield as a |
| | cyclopropyl)imidazo[1,2-a]pyridine-6-carboxamide hydrochloride | white solid. |
| | | LCMS m/z = 403.0 [M + H]⁺ |
| | 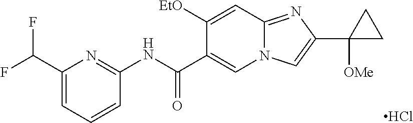 [SMILES: CCOc1cc2nc(C3(OC)CC3)cn2cc1C(=O)Nc1cccc(C(F)F)n1 ; Cl] | ¹H NMR (400 MHz, MeOH- d₄) δ: 1.16-1.18 (m, 2H), 1.22-1.24 (m, 2H), 1.64 (t, 3H), 3.41 (s, 3H), 4.34-4.39 (m, 2H), 6.47-6.75 (m, 1H), 6.95 (s, 1H), 7.44 (d, 1H), 7.80 (s, 1H), 7.97 (t, 1H), 8.44 (d, 1H), 9.10 (s, 1H) |
| | RCO₂H: 7-ethoxy-2-(1-methoxycyclopropyl)imidazo[1,2-a]pyridine-6-carboxylic | |
| | acid (Preparation 301). | |
| | RNH₂: 6-(difluoromethyl)pyridin-2-amine | |
| | Prep-HPLC-A, 20-40%. | |
| 391 | 7-Ethoxy-2-(tetrahydro-2H-pyran-4-yl)-N-(6-(tetrahydrofuran-3- | 6 mg, 7 % yield, as a |
| | yl)pyridin-2-yl)imidazo[1,2-a]pyridine-6-carboxamide hydrochloride | yellow solid. LCMS m/z = |
| | | 437.1 [M + H]⁺ IHNMR |
| | 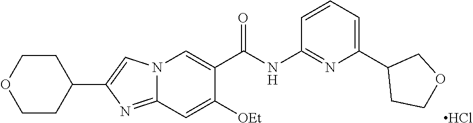 [SMILES: CCOc1cc2nc(C3CCOCC3)cn2cc1C(=O)Nc1cccc(C2CCOC2)n1 ; Cl] | (500 MHz, MeOH-d₄) δ: 1.50-1.60 (m, 3H), 1.70- 1.80 (m, 2H), 2.00-2.10 (m, 2H), 2.20-2.25 (m, 1H), 2.60-2.70 (m, 1H), 3.10- 3.20 (m, 1H), 3.60-3.65 (m, 2H), 3.85-3.90 (m, 1H), 3.95-4.00 (m, 1H), 4.00-4.20 (m, 4H), 4.20-4.25 (m, 1H), |
| | RCO₂H: 7-ethoxy-2-(tetrahydro-2H-pyran-4-yl)imidazo[1,2-a]pyridine-6- | 4.50 (q, 2H), 7.37 (s, 1H), |
| | carboxylic acid (Preparation 360) | 7.50-7.55 (m, 1H), 7.80-7.85 |
| | RNH₂: 6-(tetrahydrofuran-3-yl)pyridin-2-amine | (m, 1H), 7.96 (s, 1H), 8.20- |
| | Prep-HPLC-D, 22-42%. | 8.30 (m, 1H), 9.23 (s, 1H) |
| 392 | N-(6-(1,2-difluoroethyl)pyridin-2-yl)-7-ethoxy-2-(tetrahydro-2H-pyran-4- | 21 mg, 13% yield, as an |
| | yl)imidazo[1,2-a]pyridine-6-carboxamide hydrochloride | off-white solid. LCMS m/z = |
| | | 431.1 [M + H]⁺ ¹H NMR |
| | 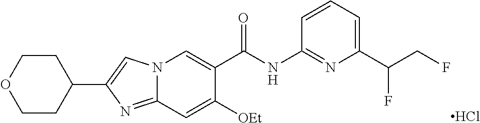 [SMILES: CCOc1cc2nc(C3CCOCC3)cn2cc1C(=O)Nc1cccc(C(F)CF)n1 ; Cl] | (500 MHz, MeOH-d₄) δ: 1.60-1.70 (m, 3H), 1.80- 1.90 (m, 2H), 2.00-2.10 (m, 2H), 3.10-3.20 (m, 1H), 3.60-3.70 (m, 2H), 4.00- 4.10 (m, 2H), 4.40-4.50 (m, 2H), 4.70-4.80 (m, 2H), 5.60-5.80 (m, 1H), 7.33 (s, 1H), 7.35-7.40 (m, 1H), |
| | RCO₂H: 7-ethoxy-2-(tetrahydro-2H-pyran-4-yl)imidazo[1,2-a]pyridine-6- | 7.90-8.00 (m, 2H), 8.30- |
| | carboxylic acid (Preparation 360) | 8.40 (m, 1H), 9.22 (s, 1H) |
| | RNH₂: 6-(1,2-difluoroethyl)pyridin-2-amine (Preparation X) | |
| | Prep-HPLC-D, 22-42%. | |
| 393 | 8-Fluoro-7-isopropoxy-N-(6-(1-methyl-1H-pyrazol-4-yl)pyridin-2-yl)-2-(1- | 35.1 mg, 48% |
| | methyl-2-oxabicyclo[2.1.1]hexan-4-yl)imidazo[1,2-a]pyridine-6-carboxamide | LCMS m/z = 491.1 [M + H]⁺ |
| | | ¹H NMR (500 MHz, MeOH- |
| | 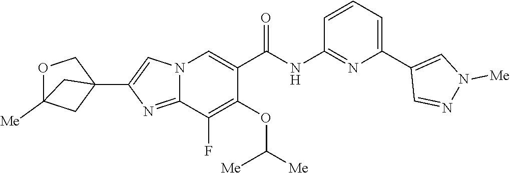 [SMILES: CC(C)Oc1c(C(=O)Nc2cccc(-c3cnn(C)c3)n2)cn2cc(C34COC(C)(C3)C4)nc2c1F] | d₄) δ: 1.51 (s, 3H), 1.56 (d, 6H), 1.80-1.90 (m, 2H), 2.10-2.20 (m, 2H), 3.96 (s, 3H), 4.04 (s, 2H), 4.94-5.00 (m, 1H), 7.41 (d, 1H), 7.79 (t, 1H), 7.88 (d, 1H), 7.99 (s, H), 8.08-8.12 (m, 2H), 8.99 (s, 1H), |
| | RCO₂H: 8-fluoro-7-isopropoxy-2-(1-methyl-2-oxabicyclo[2.1.1]hexan-4- | |
| | yl)imidazo[1,2-a]pyridine-6-carboxylic acid (Preparation 127) | |
| | RNH₂: 6-(1-methyl-1H-pyrazol-4-yl)pyridin-2-amine (Preparation X) | |
| | prep-HPLC-K, 42-72% | |
| 394 | N-(6-(difluoromethyl)pyridin-2-yl)-8-isopropoxy-2-(tetrahydro-2H-pyran-3- | 10 mg, 7.1% LCMS m/z = |
| | yl)imidazo[1,2-a]pyridine-6-carboxamide | 431.2 [M + H]⁺; ¹H NMR |
| | | (400 MHz, DMSO-d₆) δ: |
| | 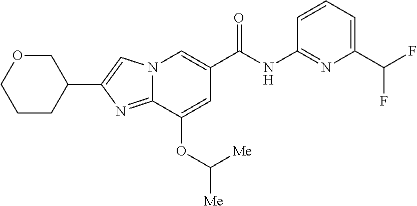 [SMILES: CC(C)Oc1cc(C(=O)Nc2cccc(C(F)F)n2)cn2cc(C3CCCOC3)nc12] | 1.38 (d, 6H), 1.65-1.68 (m, 2H), 1.78-1.79 (m, 1H), 2.06-2.07 (m, 1H), 2.93- 2.96 (m, 1H), 3.41-3.46 (m, 2H), 3.84-3.87 (m, 1H), 4.00-4.03 (m, 1H), 4.98- 5.03 (m, 1H), 7.07-6.80 (m, 1H), 7.18 (s, 1H), 7.48 (d, 1H), 7.83 (s, 1H), 8.04-8.08 (m, 1H), 8.34 (d, 1H), 8.91 (d, 1H), 11.12 (s, 1H) |
| | RCO₂H: 8-isopropoxy-2-(tetrahydro-2H-pyran-3 -yl)imidazo[1,2-a]pyridine-6- | |
| | carboxylic acid (Preparation 303). | |
| | RNH₂: 6-(difluoromethyl)pyridin-2-amine | |
| | Prep-HPLC-K, 47-68% | |
| 395 | 2-(3-Oxabicyclo[3.1.0]hexan-6-yl)-N-(1-(difluoromethyl)-1H-pyrazol-3-yl)-7- | 12.3 mg, 17.7% |
| | isopropoxyimidazo[1,2-a]pyrimidine-6-carboxamide | LCMS m/z = 419.1 [M + H]⁺ |
| | | ¹H NMR (500 MHz, |
| | 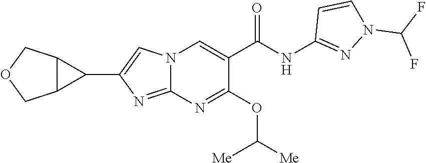 [SMILES: CC(C)Oc1nc2nc(C3C4COCC43)cn2cc1C(=O)Nc1ccn(C(F)F)n1] | CDCl₃) δ: 1.57 (s, 6H), 1.94 (t, 1H), 2.26 (s, 2H), 3.84 (d, 2H), 4.02 (d, 2H), 5.75- 5.81 (m, 1H), 6.95-7.23 (m, 2H), 7.77 (d, 1H), 9.15 (s, 1H), 10.02 (s, 1H) |
| | RCO₂H: 2-(3-oxabicyclo[3.1.0]hexan-6-yl)-7-isopropoxyimidazo[1,2- | |
| | a]pyrimidine-6-carboxylic acid (Preparation 362). | |
| | RNH₂: 1-(difluoromethyl)-1H-pyrazol-3-amine | |
| | Prep-HPLC-K, 32-62% | |
| 396 | 2-(3-Oxabicyclo[3.1.0]hexan-6-yl)-7-isopropoxy-N-(1-methyl-1H-pyrazol-3- | 15 mg, 23.8% |
| | yl)imidazo[1,2-a]pyrimidine-6-carboxamide formate | LCMS m/z = 383.1 [M + H]⁺ |
| | | ¹H NMR (500 MHz, |
| | 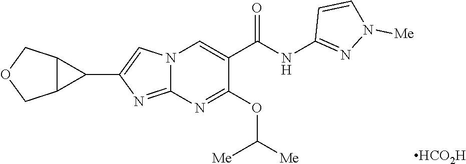 [SMILES: CC(C)Oc1nc2nc(C3C4COCC43)cn2cc1C(=O)Nc1ccn(C)n1 ; "·HCO2H"] | CDCl₃) δ: 1.57 (d, 6H), 1.94 (s, 1H), 2.26 (s, 2H), 3.83-3.86 (m, 5H), 4.02 (d, 2H), 5.74-5.81 (m, 1H), 6.75 (d, 1H), 7.26 (s, 1H), 7.30 (d, 1H), 9.14 (s, 1H), 10.08 (s, 1H) |
| | RCO₂H: 2-(3-oxabicyclo[3.1.0]hexan-6-yl)-7-isopropoxyimidazo[1,2- | |
| | a]pyrimidine-6-carboxylic acid (Preparation 362). | |
| | RNH₂: 1-methyl-1H-pyrazol-3-amine | |
| | prep-HPLC-C, 23-44% | |
| 397 | 7-Isopropoxy-2-(1-methyl-2-oxabicyclo[2.1.1]hexan-4-yl)-N-(4- | 19.3 mg, 32.8% |
| | (trifluoromethyl)thiazol-2-yl)imidazo[1,2-a]pyrimidine-6-carboxamide | LCMS m/z = 468.1 [M + H]⁺ |
| | | ¹H NMR (500 MHz, MeOH- |
| | 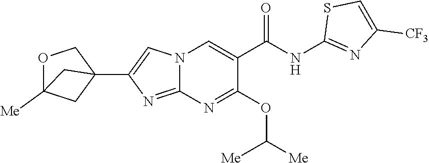 [SMILES: CC(C)Oc1nc2nc(C34COC(C)(C3)C4)cn2cc1C(=O)Nc1nc(C(F)(F)F)cs1] | d₄) δ: 1.50 (s, 3H), 1.59 (d, 6H), 1.80-1.90 (m, 2H), 2.10-2.20 (m, 2H), 4.01 (s, 2H), 5.60-5.70 (m, 1H), 7.62 (s, 1H), 7.78 (s, 1H), 9.37 (s, 1H). |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | RNH₂: 4-(trifluoromethyl)thiazol-2-amine. Prep-HPLC-K, 42-72% | |
| 398 | N-(6-fluoropyrazolo[1,5-a]pyrimidin-3-yl)-7-isopropoxy-2-(1-methyl-2- | 13 mg, 24.7% |
| | oxabicyclo[2.1.1]hexan-4-yl)imidazo[1,2-a]pyrimidine-6-carboxamide | LCMS m/z = 452.1 [M + H]⁺ |
| | | ¹H NMR (500 MHz, MeOH- |
| | 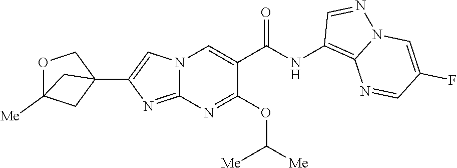 [SMILES: CC(C)Oc1nc2nc(C34COC(C)(C3)C4)cn2cc1C(=O)Nc1cnn2cc(F)cnc12] | d₄) δ: 1.50 (s, 3H), 1.65 (d, 6H), 1.80-1.90 (m, 2H), 2.00- 2.10 (m, 2H), 4.01 (s, 2H), 5.70-5.80 (m, 1H), 7.64 (s, 1H), 8.50-8.60 (m, 1H), 8.79 (s, 1H), 9.00-9.10 (m, 1H), 9.40 (s, 1H) |
| | RCO₂H: 7-isopropoxy-2-(1-methyl-2-oxabicyclo[2.1.1]hexan-4- | |
| | yl)imidazo[1,2-a]pyrimidine-6-carboxylic acid (Preparation 128). | |
| | RNH₂: 6-fluoropyrazolo[1,5-a]pyrimidin-3-amine (Preparation X) | |
| | Prep-HPLC-H, 45-72% | |
| 399 | N-(2-chloro-3-fluorophenyl)-7-isopropoxy-2-(1-methyl-2-oxabicyclo[2.1.1] | 27.2 mg, 8.8% |
| | hexan-4-yl)imidazo[1,2-a]pyrimidine-6-carboxamide | LCMS m/z = 445.1 [M + H]⁺ |
| | | ¹H NMR (400 MHz, MeOH- |
| | 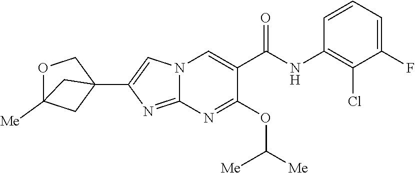 [SMILES: CC(C)Oc1nc2nc(C34COC(C)(C3)C4)cn2cc1C(=O)Nc1cccc(F)c1Cl] | d₄) δ: 1.50 (s, 3H), 1.58 (d, 6H), 1.80-1.90 (m, 2H), 2.10-2.20 (m, 2H), 4.01 (s, 2H), 5.80-5.90 (m, 1H), 7.10-7.20 (m, 1H), 7.30- 7.40 (m, 1H), 7.62 (s, 1H), 8.30 (d, 1H), 9.42 (s, 1H) |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | RNH₂: 2-chloro-3-fluoroaniline | |
| | Prep-HPLC-I, 49-69% | |
| 400 | 8-Isopropoxy-2-(1-methyl-2-oxabicyclo[2.1.1]hexan-4-yl)-N-(pyrazolo[1,5- | 10.1 mg, 24.6% yield, as a |
| | a]pyrimidin-7-yl)imidazo[1,2-a]pyrazine-6-carboxamide | white solid. |
| | | LCMS m/z = 434.1 [M + H]⁺ |
| | 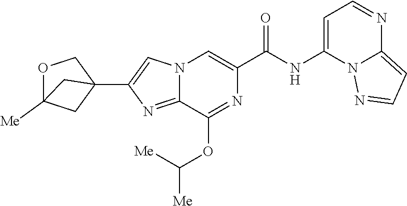 [SMILES: CC(C)Oc1nc(C(=O)Nc2ccnc3ccnn23)cn2cc(C34COC(C)(C3)C4)nc12] | ¹H NMR (500 MHz, CDCl₃) δ: 1.55 (s, 3H), 1.69 (d, 6H), 2.01-2.03 (m, 2H), 2.12-2.14 (m, 2H), 4.12 (s, 2H), 5.80-5.84 (m, 1H), 6.73 (d, 1H), 7.61 (s, 1H), 7.82 (d, 1H), 8.13 (d, 1H), 8.53 (d, 1H), 8.68 (s, 1H), 11.71 (s, 1H) |
| | RCO₂H: 8-isopropoxy-2-(1-methyl-2-oxabicyclo[2.1.1]hexan-4-yl)imidazo[1,2- | |
| | a]pyrazine-6-carboxylic acid as the starting carboxylic acid (Preparation 305) | |
| | RNH₂: pyrazolo[1,5-a]pyrimidin-7-amine | |
| | Prep-HPLC-K, 35-65% | |
| 401 | 8-Isopropoxy-N-(4-methoxypyrazolo[1,5-a]pyridin-3-yl)-2-(1-methyl-2- | 22.8 mg, 52.1% yield, as a |
| | oxabicyclo[2.1.1]hexan-4-yl)imidazo[1,2-a]pyrazine-6-carboxamide | white solid. |
| | | LCMS m/z = 463.1 [M + H]⁺ |
| | 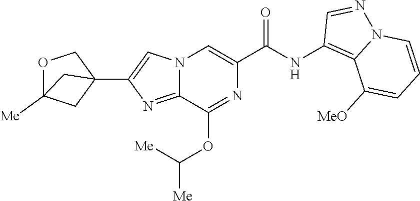 [SMILES: COc1cccn2ncc(NC(=O)c3cn4cc(C56COC(C)(C5)C6)nc4c(OC(C)C)n3)c12] | ¹H NMR (500 MHz, CDCl₃) δ: 1.54 (s, 3H), 1.63 (d, 6H), 2.02-2.02 (m, 2H), 2.11-2.13 (m, 2H), 4.04 (s, 3H), 4.12 (s, 2H), 5.77-5.83 (m, 1H), 6.38 (d, 1H), 6.64 (t, 1H), 7.59 (s, 1H), 8.06 (d, 1H), 8.66 (s, 1H), 8.84 (s, 1H), 10.20 (s, 1H) |
| | RCO₂H: 8-isopropoxy-2-(1-methyl-2-oxabicyclo[2.1.1]hexan-4-yl)imidazo[1,2- | |
| | a]pyrazine-6-carboxylic acid (Preparation 305) | |
| | RNH₂: 4-methoxypyrazolo[1,5-a]pyridin-3-amine (Preparation X). | |
| | Prep-HPLC-K, 36-66% | |
| 402 | 2-(3-Cyanobicyclo[1.1.1]pentan-1-yl)-N-(6-(difluoromethyl)pyridin-2- | 13.5 mg, 23.1% yield as a |
| | yl)-7-isopropoxyimidazo[1,2-a]pyridine-6-carboxamide | yellow solid |
| | | LCMS m/z = 438.2 [M + H]⁺ |
| | 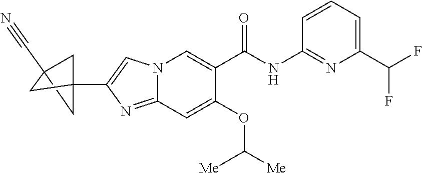 [SMILES: CC(C)Oc1cc2nc(C34CC(C#N)(C3)C4)cn2cc1C(=O)Nc1cccc(C(F)F)n1] | ¹H NMR (500 MHz, MeOH-d₄) δ: 1.57 (d, 6H), 2.61 (s, 6H), 4.95-5.00 (m, 1H), 6.48-6.72 (m, 1H), 6.96 (s, 1H), 7.42 (d, 1H), 7.67 (s, 1H), 7.95-7.99 (m, 1H), 8.41 (d, 1H), 9.08 (s, 1H). |
| | RCO₂H: 2-(3-cyanobicyclo[1.1.1]pentan-1-yl)-7-isopropoxyimidazo[1,2- | |
| | a]pyridine-6-carboxylic acid (Preparation 304) | |
| | RNH₂: (6-difluoromethyl)pyridine-2-amine. | |
| | Prep-HPLC-K, 43-73% | |
| 403 | 2-(3-Cyanobicyclo[1.1.1]pentan-1-y l)-7-isopropoxy-N-(2-methoxypyridin-3- | 13.3 mg, 24% yield as a |
| | yl)imidazo[1,2-a]pyridine-6-carboxamide | brown solid |
| | | LCMS m/z = 418.2 [M + H]⁺ |
| | 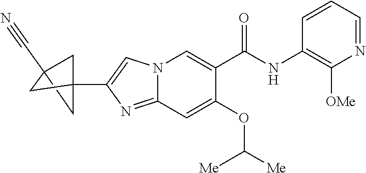 [SMILES: COc1ncccc1NC(=O)c1cn2cc(C34CC(C#N)(C3)C4)nc2cc1OC(C)C] | ¹H NMR (500 MHz, MeOH-d₄) δ: 1.59 (d, 6H), 2.62 (s, 6H), 4.09 (s, 3H), 5.08-5.02 (m, 1H), 6.99- 7.02 (m, 2H), 7.70 (s, 1H), 7.91-7.89 (m, 1H), 8.78- 8.80 (m, 1H), 9.15 (s, 1H) |
| | RCO₂H: 2-(3-cyanobicyclo[1.1.1]pentan-1-yl)-7-isopropoxyimidazo[1,2- | |
| | a]pyridine-6-carboxylic acid (Preparation 304) | |
| | RNH₂: 2-methoxypyridin-3-amine | |
| | Prep-HPLC-K, 40-70% | |
| 404 | 2-(3-Cyanobicyclo[1.1.1]pentan-1-yl)-7-isopropoxy-N-(1-methyl-1H-pyrazol-3- | 12.3 mg, 24% yield as a |
| | yl)imidazo[1,2-a]pyridine-6-carboxamide | white solid |
| | | LCMS m/z = 391.2 [M + H]⁺ |
| | 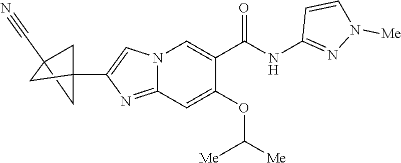 [SMILES: CC(C)Oc1cc2nc(C34CC(C#N)(C3)C4)cn2cc1C(=O)Nc1ccn(C)n1] | ¹H NMR (500 MHz, MeOH-d₄) □: 1.54 (d, 6H), 2.60 (s, 6H), 3.84 (s, 3H), 4.92-4.97 (m, 1H), 6.67 (s, 1H), 6.96 (s, 1H), 7.51 (s, 1H), 7.66 (s, 1H), 9.01 (s, 1H) |
| | RCO₂H: 2-(3-cyanobicyclo[1.1.1]pentan-1-yl)-7-isopropoxyimidazo[1,2- | |
| | a]pyridine-6-carboxylic acid (Preparation 304). | |
| | RNH₂: 1-methylpyrazol-3-amine Prep-HPLC-K, 29-59% | |
| 405 | 7-Cyclobutoxy-2-(1-methyl-2-oxabicyclo[2.1.1]hexan-4-yl)-N-(6- | 20 mg, 28.7% yield as a |
| | methylpyrazolo[1,5-a]pyrimidin-3-yl)imidazo[1,2-a]pyridine-6-carboxamide | yellow solid |
| | | LCMS m/z = 459.2 [M + H]⁺ |
| | 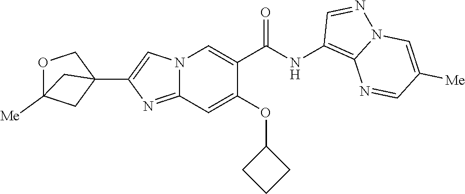 [SMILES: Cc1cnc2c(NC(=O)c3cn4cc(C56COC(C)(C5)C6)nc4cc3OC3CCC3)cnn2c1] | ¹H NMR (500 MHz, CDCl₃) δ: 1.55 (s, 3H), 1.77-2.09 (m, 6H), 2.40 (s, 3H), 2.60-2.72 (m, 4H), 4.08 (s, 2H), 4.90-4.97 (m, 1H), 6.85 (s, 1H), 7.38 (s, 1H), 8.31 (s, 1H), 8.40- 8.42 (m, 1H), 8.85 (s, 1H), 9.07 (s, 1H), 10.52 (s, 1H) |
| | RCO₂H: 7-cyclobutoxy-2-(1-methyl-2-oxabicyclo[2.1.1]hexan-4- | |
| | yl)imidazo[1,2-a]pyridine-6-carboxylic acid (Preparation 343) | |
| | RNH₂: 6-methylpyrazolo[1,5-a]pyrimidin-3-amine. Prep-HPLC-K, 32-62% | |
| 406 | 7-Cyclobutoxy-2-(1-methyl-2-oxabicyclo[2.1.1]hexan-4-yl)-N-(pyrazolo[1,5- | 30.0 mg, 44.3% yield as a |
| | a]pyrimidin-3-yl)imidazo[1,2-a]pyridine-6-carboxamide | yellow solid |
| | | LCMS m/z = 445.0 [M + H]⁺ |
| | 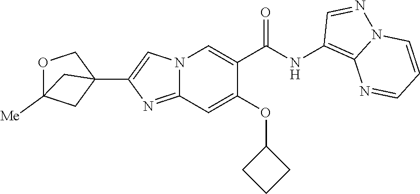 [SMILES: CC12CC(c3cn4cc(C(=O)Nc5cnn6cccnc56)c(OC5CCC5)cc4n3)(CO1)C2] | ¹H NMR (500 MHz, CDCl₃) δ: 1.54 (s, 3H), 1.77-2.09 (m, 6H), 2.60-2.72 (m, 4H), 4.08 (s, 2H), 4.92- 4.96 (m, 1H), 6.82-6.86 (m, 2H), 7.39 (s, 1H), 8.41 (d, 1H), 8.64 (d, 1H), 8.96 (s, 1H), 9.08 (s, 1H), 10.54 (s, 1H) |
| | RCO₂H: 7-cyclobutoxy-2-(1-methyl-2-oxabicyclo[2.1.1]hexan-4- | |
| | yl)imidazo[1,2-a]pyridine-6-carboxylic acid (Preparation 343) | |
| | RNH₂: pyrazolo[1,5-a]pyrimidin-3-amine | |
| | Prep-HPLC-K, 27-57% | |
| 407 | 8-Fluoro-7-isopropoxy-N-(6-methoxypyrazolo[1,5-a]pyrimidin-3-yl)-2-(1- | 17.1 mg, 22.8% yield as a |
| | methyl-2-oxabicyclo[2.1.1]hexan-4-yl)imidazo[1,2-a]pyridine-6-carboxamide | white solid |
| | | LCMS m/z = 481.1 [M + H]⁺ |
| | 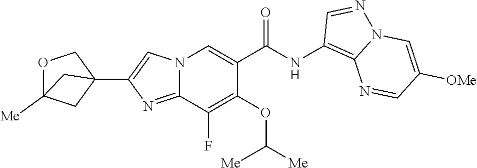 [SMILES: COc1cnc2c(NC(=O)c3cn4cc(C56COC(C)(C5)C6)nc4c(F)c3OC(C)C)cnn2c1] | ¹H NMR (400 MHz, CDCl₃) δ: 1.55 (s, 3H), 1.58 (d, 6H), 10.52 (s, 1H), 1.96- 2.00 (m, 2H), 2.12-2.14 (m, 2H), 3.91 (s, 3H), 4.10 (s, 2H), 5.03-5.10 (m, 1H), 7.48 (d, 1H), 8.16 (d, 1H), 8.32 (d, 1H), 8.78 (s, 1H), 8.92 (s, 1H) |
| | RCO₂H: 7-cyclobutoxy-8-fluoro-2-(1-methyl-2-oxabicyclo[2.1.1]hexan-4- | |
| | yl)imidazo[1,2-a]pyridine-6-carboxylic acid (Preparation 321) | |
| | RNH₂: 6-methoxypyrazolo[1,5-a]pyrimidin-3-amine (Preparation X). | |
| | Prep-HPLC-K, 33-63% | |
| 408 | N-(4-fluoropyrazolo[1,5-a]pyridin-3-yl)-7-isopropoxy-2-(1-methyl-2- | 31.1 mg, 43.8% yield as a |
| | oxabicyclo[2.1.1]hexan-4-yl)imidazo[1,2-a]pyrimidine-6-carboxamide | white solid |
| | | LCMS m/z = 451.2 [M + H]⁺ |
| | 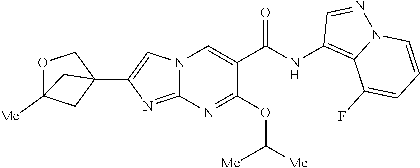 [SMILES: CC(C)Oc1nc2nc(C34COC(C)(C3)C4)cn2cc1C(=O)Nc1cnn2cccc(F)c12] | ¹H NMR (500 MHz, CDCl₃) δ: 1.54 (s, 3H), 1.57 (d, 6H), 1.95-1.98 (m, 2H), 2.10-2.12 (m, 2H), 4.09 (s, 2H), 5.94-5.99 (m, 1H), 6.67-6.70 (m, 1H), 6.79- 6.83 (m, 1H), 7.29 (s, 1H), 8.25 (d, 1H), 8.82 (s, 1H), 9.26 (s, 1H), 10.13 (s, 1H) |
| | RCO₂H: 7-isopropoxy-2-(1-methyl-2-oxabicyclo[2.1.1]hexan-4- | |
| | yl)imidazo[1,2-a]pyrimidine-6-carboxylic acid (Preparation 128). | |
| | RNH₂: 4-fluoropyrazolo[1,5-a]pyridin-3-amine. Prep-HPLC-K, 32-62% | |
| 409 | N-([1,2,4]triazolo[4,3-a]pyridin-8-yl)-7-isopropoxy-2-(1-methyl-2- | 12.0 mg, 18.5% yield as a |
| | oxabicyclo[2.1.1]hexan-4-yl)imidazo[1,2-a]pyrimidine-6-carboxamide | white solid |
| | | LCMS m/z = 434.1 [M + H]⁺ |
| | 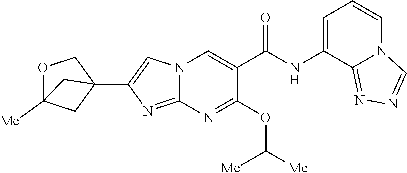 [SMILES: CC(C)Oc1nc2nc(C34COC(C)(C3)C4)cn2cc1C(=O)Nc1cccn2cnnc12] | ¹H NMR (500 MHz, MeOD-d₄) δ: 1.50 (s, 3H), 1.70 (d, 6H), 1.86-1.90 (m, 2H), 2.09-2.13 (m, 2H), 4.00 (s, 2H), 5.74-5.82 (m, 1H), 6.94 (t, 1H), 7.60 (s, 1H), 8.18 (d, 1H), 8.28 (d, 1H), 9.20 (s, 1H), 9.40 (s, 1H) |
| | RCO₂H: 7-isopropoxy-2-(1-methyl-2-oxabicyclo[2.1.1]hexan-4-yl)imidazo[1,2- | |
| | a]pyrimidine-6-carboxylic acid (Preparation 128). | |
| | RNH₂: [1,2,4]triazolo[4,3-a]pyridin-8-amine. prep-HPLC-J | |
| 410 | N-([1,2,4]triazolo[1,5 -a]pyridin-5-yl)-7-isopropoxy-2-(1-methyl-2- | 10 mg, 17% as a white solid |
| | oxabicyclo[2.1.1]hexan-4-yl)imidazo[1,2-a]pyrimidine-6-carboxamide | LCMS m/z = 434.1 [M + H]⁺ |
| | | ¹H NMR (500 MHz, |
| | 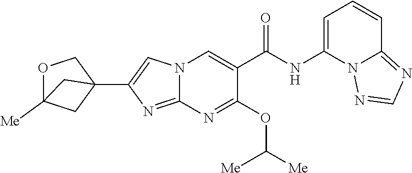 [SMILES: CC(C)Oc1nc2nc(C34COC(C)(C3)C4)cn2cc1C(=O)Nc1cccc2ncnn12] | MeOH-d₄) δ: 1.51 (s, 3H), 1.71 (d, 6H), 1.86-1.90 (m, 2H), 2.09-2.13 (m, 2H), 4.01 (s, 2H), 5.90-5.83 (m, 1H), 7.53-7.50 (m, 1H), 7.62 (s, 1H), 7.72-7.76 (m, 1H), 8.12-8.15 (m, 1H), 8.52 (s, 1H), 9.48 (s, 1H) |
| | RCO₂H: 7-isopropoxy-2-(1-methyl-2-oxabicyclo[2.1.1]hexan-4- | |
| | yl)imidazo[1,2-a]pyrimidine-6-carboxylic acid (Preparation 128). | |
| | RNH₂: [1,2,4]triazolo[1,5-a]pyridin-5-amine. Prep-HPLC-J, 30-60% | |
| 411 | N-(6-(difluoromethoxy)pyridin-2-yl)-7-isopropoxy-2-(1-methyl-2- | 17.3 mg, 29.9% as an off- |
| | oxabicyclo[2.1.1]hexan-4-yl)imidazo[1,2-a]pyrimidine-6-carboxamide | white solid |
| | | LCMS m/z = 460.1 [M + H]⁺ |
| | 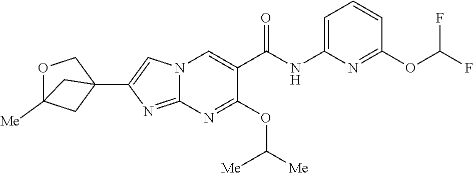 [SMILES: CC(C)Oc1nc2nc(C34COC(C)(C3)C4)cn2cc1C(=O)Nc1cccc(OC(F)F)n1] | ¹H NMR (500 MHz, MeOH- d₄): 1.50 (s, 3H), 1.60 (d, 6H), 1.86-1.90 (m, 2H), 2.09-2.13 (m, 2H), 4.01 (s, 1H), 5.63-5.69 (m, 1H), 6.76 (d, 1H), 7.30-7.60 (m, 1H), 7.62 (s, 1H), 7.90 (t, 1H), 8.10 (d, 1H), 9.37 (s, 1H) |
| | RCO₂H: 7-isopropoxy-2-(1-methyl-2-oxabicyclo[2.1.1]hexan-4- | |
| | yl)imidazo[1,2-a]pyrimidine-6-carboxylic acid (Preparation 128). | |
| | RNH₂: (6-difluoromethoxy)pyridin-2-amine. Prep-HPLC-K, 42-72% | |
| 412 | 7-Isopropoxy-2-(1-methyl-2-oxabicyclo[2.1.1]hexan-4-yl)-N-(6- | 31.1 mg, 43.8% as a white |
| | (trifluoromethoxy)pyridin-2-yl)imidazo[1,2-a]pyrimidine-6-carboxamide | solid |
| | | LCMS m/z = 478.0 [M + H]⁺ |
| | 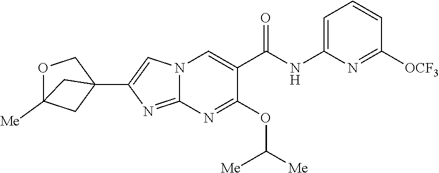 [SMILES: CC(C)Oc1nc2nc(C34COC(C)(C3)C4)cn2cc1C(=O)Nc1cccc(OC(F)(F)F)n1] | ¹H NMR (500 MHz, CDCl₃) δ: 1.54 (s, 3H), 1.59 (d, 6H), 10.55 (s, 1H), 1.95- 1.97 (m, 2H), 2.09-2.11 (m, 2H), 4.08 (s, 2H), 5.74-5.80 (m, 1H), 6.77 (d, 1H), 7.29 (s, 1H), 7.84 (t, 1H), 8.21 (d, 1H), 9.19 (s, 1H) |
| | RCO₂H: 7-isopropoxy-2-(1-methyl-2-oxabicyclo[2.1.1]hexan-4- | |
| | yl)imidazo[1,2-a]pyrimidine-6-carboxylic acid (Preparation1 28). | |
| | RNH₂: (6-trifluoromethoxy)pyridin-2-amine. Prep-HPLC-K, 55-85% | |
| 413 | 7-Isopropoxy-N-(6-(1-methyl-1H-pyrazol-4-yl)pyridin-2-yl)-2-(1-methyl-2- | LCMS m/z = 474.2 [M + H]⁺ |
| | oxabicyclo[2.1.1]hexan-4-yl)imidazo[1,2-a]pyrimidine-6-carboxamide | ¹H NMR (500 MHz, MeOH- |
| | | d₄) δ: 1.50 (s, 3H), 1.66 (br |
| | 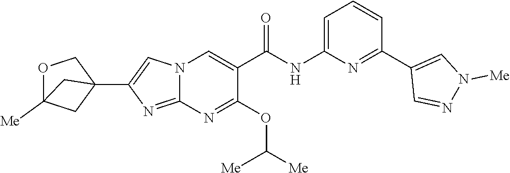 [SMILES: CC(C)Oc1nc2nc(C34COC(C)(C3)C4)cn2cc1C(=O)Nc1cccc(-c2cnn(C)c2)n1] | d, 6H), 1.87 (dd, 2H), 2.12 (dd, 2H), 3.96 (s, 3H), 4.01 (s, 2H), 5.83-5.59 (m, 1H), 7.39 (d, 1H), 7.62 (s, 1H), 7.78 (t, 1H), 7.98 (s, 1H), 8.05-8.15 (m, 2H), 9.38 (s, 1H) |
| | RCO₂H: 7-isopropoxy-2-(1-methyl-2-oxabicyclo[2.1.1]hexan-4- | |
| | yl)imidazo[1,2-a]pyrimidine-6-carboxylic acid (Preparation 128). | |
| | RNH₂: 6-(1-methyl-1H-pyrazol-4-yl)pyridin-2-amine. prep-HPLC-C | |
| 649 | 7-cyclobutoxy-N-(imidazo[1,2-b]pyridazin-3-yl)-2-(1-methyl-2- | 0.9 mg, 1.3% yield. |
| | oxabicyclo[2.2.1]heptan-4-yl)imidazo[1,2-a]pyridine-6-carboxamide | LCMS m/z = 459.2 [M + H]⁺ |
| | 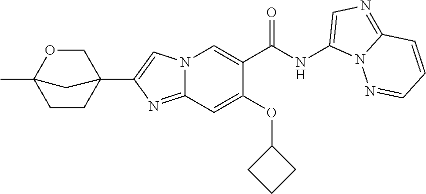 [SMILES: CC12CCC(c3cn4cc(C(=O)Nc5cnc6cccnn56)c(OC5CCC5)cc4n3)(CO1)C2] | |
| | RCO₂H-C: 7-cyclobutoxy-2-(1-methyl-2-oxabicyclo[2.2.1]heptan-4- | |
| | yl)imidazo[1,2-a]pyridine-6-carboxylic acid | |
| | RNH₂: imidazo[1,2-b]pyridazin-3-amine | |
| 650 | 7-(cyclopentyloxy)-N-(imidazo[1,2-b]pyridazin-3-yl)-2-(1-methyl-2- | 1.6 mg, 2.3% |
| | oxabicyclo[2.1.1]hexan-4-yl)imidazo[1,2-a]pyrimidine-6-carboxamide | LCMS m/z = 460.2 [M + H]⁺ |
| | 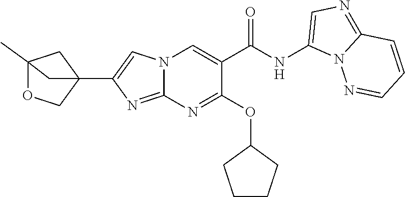 [SMILES: CC12CC(c3cn4cc(C(=O)Nc5cnc6cccnn56)c(OC5CCCC5)nc4n3)(CO1)C2] | |
| | RCO₂H: 7-(cyclopentyloxy)-2-(1-methyl-2-oxabicyclo[2.1.1]hexan-4- | |
| | yl)imidazo[1,2-a]pyrimidine-6-carboxylic acid | |
| | R-NH₂: imidazo[1,2-b]pyridazin-3-amine | |
| | prep-HPLC-J | |

** = reaction stirred at 80° C. for 12 h

### Example 414: 7-Isopropoxy-N-(1-methyl-1H-pyrazol-3-yl)-2-(1-methyl-2-oxabicyclo[2.2.1]heptan-4-yl)imidazo[1,2-a]pyridine-6-carboxamide

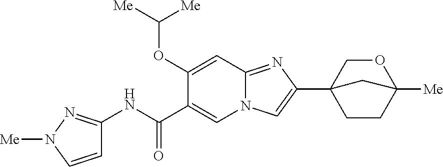 [SMILES: CC(C)Oc1cc2nc(C34CCC(C)(C3)OC4)cn2cc1C(=O)Nc1ccn(C)n1]

To 7-isopropoxy-2-(1-methyl-2-oxabicyclo[2.2.1]heptan-4-yl)imidazo[1,2-a]pyridine-6-carboxylic acid (Preparation 298). (88.8 mg, 0.199 mmol, 2NaCl) and 1-methylpyrazol-3-amine (42 mg, 0.432 mmol) was added HATU (120 mg, 0.315 mmol) and DIPEA (139 mg, 1.08 mmol) and the mixture stirred at rt for 2.5 h. Small work-up: the reaction was partitioned between EtOAc/brine and the aqueous layer extracted with additional EtOAc. The combined organics were evaporated to dryness and purified by normal phase column (SiO₂, EtOAc 100% to EtOAc/EtOH 3/1) to afford 1-(7-isopropoxy-2-(1-methyl-2-oxabicyclo[2.2.1]heptan-4-yl)imidazo[1,2-a]pyridin-6-yl)-2-(1-methyl-1H-pyrazol-3-yl)ethan-1-one as a white solid (66 mg, 81%). LCMS m/z=410.2 [M+H]⁺; ¹H NMR (400 MHz, MeOH-d₄) δ: 9.07 (s, 1H), 7.73 (s, 1H), 7.54 (d, 1H), 7.02 (s, 1H), 6.69 (d, 1H), 4.93-5.05 (m, 1H), 4.06 (dd, 1H), 3.94 (d, 1H), 3.86 (s, 3H), 1.73-2.30 (m, 6H), 1.58 (d, 6H), 1.48 (s, 3H).

### Example 415-429

The title compounds were prepared from the appropriate carboxylic (RCO₂H) and amine using an analogous method to that described for Example 414.

RCO₂H-A: 7-isopropoxy-2-(1-methyl-2-oxabicyclo[2.2.1]heptan-4-yl)imidazo[1,2-a]pyridine-6-carboxylic acid (Preparation 298); RCO₂H-B: 2-(2-oxabicyclo[2.2.1]heptan-4-yl)-7-isopropoxyimidazo[1,2-a]pyridine-6-carboxylic acid (Preparation 348); Acid C: 7-cyclobutoxy-2-(1-methyl-2-oxabicyclo[2.2.1]heptan-4-yl)imidazo[1,2-a]pyridine-6-carboxylic acid (Preparation 296); Acid D: 7-cyclobutoxy-2-(1-methyl-2-oxabicyclo[2.2.2]octan-4-yl)imidazo[1,2-a]pyridine-6-carboxylic acid (Preparation 297); Acid E: 7-ethoxy-2-(1-methyl-2-oxabicyclo[2.2.2]octan-4-yl)imidazo[1,2-a]pyrimidine-6-carboxylic acid (Preparation 356); Acid F: 7-cyclobutoxy-2-(1-methyl-2-oxabicyclo[2.1.1]hexan-4-yl)imidazo[1,2-a]pyridine-6-carboxylic acid (Preparation 343); Acid G: 8-isopropoxy-2-(1-methyl-2-oxabicyclo[2.2.2]octan-4-yl)imidazo[1,2-a]pyrazine-6-carboxylic acid (Preparation 354).

| Ex. No | Name/Structure/RCO₂H | Data |
|---|---|---|
| 415 | 2-(2-Oxabicyclo[2.2.1]heptan-4-yl)-7-isopropoxy-N-(1-methyl-1H-pyrazol-3- | 73 mg, 94% |
| | yl)imidazo[1,2-a]pyridine-6-carboxamide | LCMS m/z = 396.2 [M + H]⁺ |
| | | ¹H NMR (400 MHz, CDCl₃) δ: |
| | 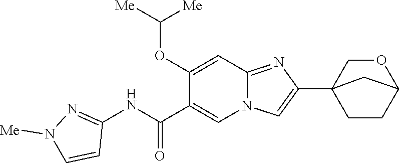 [SMILES: CC(C)Oc1cc2nc(C34CCC(C3)OC4)cn2cc1C(=O)Nc1ccn(C)n1] | 9.07 (s, 1H), 7.74 (s, 1H), 7.54 (d, 1H), 7.01 (s, 1H), 6.69 (d, 1H), 4.94-5.05 (m, 1H), 4.52 (s, 1H), 3.96 (dd, 1H), 3.83-3.90 (m, 1H), 3.86 (s, 3H), 1.83-2.21 (m, 6H), 1.58 (d, 6H). |
| | RCO₂H—B; RNH₂: 1-methyl-1H-pyrazol-3-amine | |
| 416 | 2-(2-Oxabicyclo[2.2.1]heptan-4-yl)-7-isopropoxy-N-(pyrazolo[1,5- | 64.9 mg, 75% |
| | a]pyrimidin-3-yl)imidazo[1,2-a]pyridine-6-carboxamide | LCMS m/z = 433.2 [M + H]⁺ |
| | | ¹H NMR (400 MHz, CDCl₃) |
| | 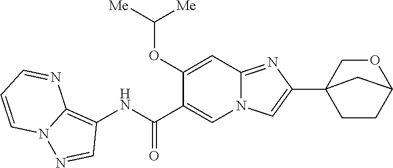 [SMILES: CC(C)Oc1cc2nc(C34CCC(C3)OC4)cn2cc1C(=O)Nc1cnn2cccnc12] | δ: 10.57 (s, 1H), 9.08 (s, 1H), 8.95 (s, 1H), 8.61 (dd, 1H), 8.40 (dd, 1H), 7.38 (s, 1H), 7.01 (s, 1H), 6.81 (dd, 1H), 4.87 (td, 1H), 4.01 (dd, 1H), 3.91 (d, 1H), 1.78-2.20 (m, 6H), 1.66 (d, 6H). |
| | RCO₂H—B; RNH₂: pyrazolo[1,5-a]pyrimidin-3-amine | |
| 417 | 7-Isopropoxy-2-(1-methyl-2-oxabicyclo[2.2.1]heptan-4-yl)-N- | 66 mg, 81% |
| | (pyrazolo[1,5-a]pyrimidin-3-yl)imidazo[1,2-a]pyridine-6-carboxamide | LCMS m/z = 447.2 [M + H]⁺ |
| | | ¹H NMR (400 MHz, MeOH- |
| | 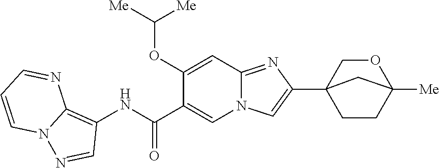 [SMILES: CC(C)Oc1cc2nc(C34CCC(C)(C3)OC4)cn2cc1C(=O)Nc1cnn2cccnc12] | d₄) δ: 9.16 (s, 1H), 8.86 (dd, 1H), 8.81 (s, 1H), 8.53 (dd, 1H), 7.72 (s, 1H), 6.96-7.07 (m, 2H), 5.06 (td, 1H), 4.07 (dd, 1H), 3.95 (d, 1H), 1.78- 2.27 (m, 6H), 1.67 (d, 6H), 1.49 (s, 3H). |
| | RCO₂H—A; RNH₂: pyrazolo[1,5-a]pyrimidin-3-amine | |
| 418 | 7-Cyclobutoxy-N-(1-(difluoromethyl)-1H-pyrazol-3-yl)-2-(1-methyl-2- | 5 mg, 56% |
| | oxabicyclo[2.2.1]heptan-4-yl)imidazo[1,2-a]pyridine-6-carboxamide | LCMS m/z = 458.3 [M + H]⁺ |
| | | ¹H NMR (400 MHz, MeOH- |
| | 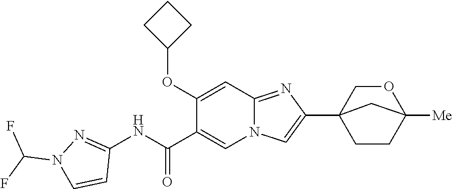 [SMILES: CC12CCC(c3cn4cc(C(=O)Nc5ccn(C(F)F)n5)c(OC5CCC5)cc4n3)(CO1)C2] | d₄) δ: 9.04 (s, 1H), 8.02 (d, 1H), 7.68 (s, 1H), 7.23-7.60 (m, 1H), 7.02 (d, 1H), 6.80 (s, 1H), 5.02 (quin, 1H), 4.05 (dd, 1H), 3.93 (d, 1H), 2.59-2.75 (m, 2H), 2.31-2.49 (m, 2H), 1.76-2.23 (m, 9H), 1.48 (s, 3H). |
| | RCO₂H—C; —RNH₂: 1-(difluoromethyl)-1H-pyrazol-3-amine | |
| 419 | 7-Cyclobutoxy-N-(5-fluoro-1-methyl-1H-pyrazol-3-yl)-2-(1-methyl-2- | 6 mg, 68% |
| | oxabicyclo[2.2.1]heptan-4-yl)imidazo[1,2-a]pyridine-6-carboxamide | LCMS m/z = 440.2 [M + H]⁺ |
| | | ¹H NMR (400 MHz, MeOH- |
| | 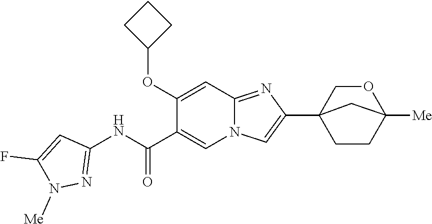 [SMILES: Cn1nc(NC(=O)c2cn3cc(C45CCC(C)(C4)OC5)nc3cc2OC2CCC2)cc1F] | d₄) δ: 9.02 (s, 1H), 7.67 (s, 1H), 6.79 (s, 1H), 6.34 (d, 1H), 5.01 (quin, 1H), 4.05 (dd, 1H), 3.93 (d, 1H), 3.70 (d, 3H), 2.60-2.76 (m, 2H), 2.32-2.49 (m, 2H), 1.78-2.24 (m, 8H), 1.48 (s, 3H). |
| | RCO₂H—C; RNH₂: 5-fluoro-1-methyl-1H-pyrazol-3-amine | |
| 420 | 7-Cyclobutoxy-N-(1-ethyl-2-oxo-1,2-dihydropyridin-3-yl)-2-(1-methyl-2- | 33 mg, 70% |
| | oxabicyclo[2.2.2]octan-4-yl)imidazo[1,2-a]pyridine-6-carboxamide | LCMS m/z = 477.3 [M + H]⁺ |
| | | ¹H NMR (400 MHz, MeOH- |
| | 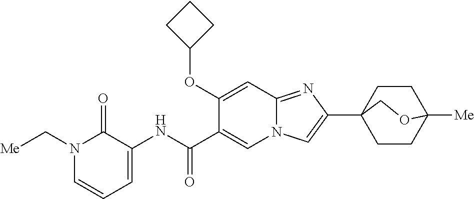 [SMILES: CCn1cccc(NC(=O)c2cn3cc(C45CCC(C)(CC4)OC5)nc3cc2OC2CCC2)c1=O] | d₄) δ: 9.01 (s, 1H), 8.47 (dd, 1H), 7.49 (s, 1H), 7.28 (dd, 1H), 6.68 (s, 1H), 6.19-6.38 (m, 1H), 4.92 (t, 1H), 4.03 (q, 2H), 3.90-3.98 (m, 2H), 2.48- 2.64 (m, 4H), 1.67-2.14 (m, 10H), 1.29 (t, 3H), 1.05 (s, 3H). |
| | RCO₂H—D; RNH₂: 3-amino-1-ethylpyridin-2(1H)-one | |
| 421 | 7-Cyclobutoxy-N-(1-methyl-2-oxo-1,2-dihydropyridin-3-yl)-2-(1-methyl-2- | 44 mg, 72% |
| | oxabicyclo[2.2.2]octan-4-yl)imidazo[1,2-a]pyridine-6-carboxamide | LCMS m/z = 463.2 [M + H]⁺ |
| | | ¹H NMR (400 MHz, MeOH- |
| | 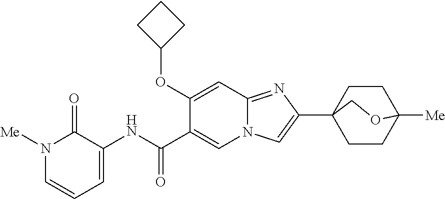 [SMILES: Cn1cccc(NC(=O)c2cn3cc(C45CCC(C)(CC4)OC5)nc3cc2OC2CCC2)c1=O] | d₄) δ: 8.99 (s, 1H), 8.48 (dd, 1H), 7.48 (s, 1H), 7.25 (dd, 1H), 6.66 (s, 1H), 6.27 (t, 1H), 4.90 (quin, 1H), 3.94 (s, 2H), 3.55 (s, 3H), 2.39-2.62 (m, 4H), 1.59-2.14 (m, 10H), 1.05 (s, 3H). |
| | RCO₂H—D; RNH₂: 3-amino-1-methylpyridin-2(1H)-one | |
| 422 | 7-Cyclobutoxy-N-(1-methyl-2-oxo-1,2-dihydropyridin-3-yl)-2-(1-methyl-2- | 94 mg, 76% |
| | oxabicyclo[2.2.1]heptan-4-yl)imidazo[1,2-a]pyridine-6-carboxamide | LCMS m/z = 449.2 [M + H]⁺ |
| | | ¹H NMR (400 MHz, MeOH- |
| | 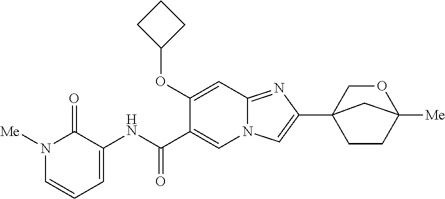 [SMILES: Cn1cccc(NC(=O)c2cn3cc(C45CCC(C)(C4)OC5)nc3cc2OC2CCC2)c1=O] | d₄) δ: 10.79 (s, 1H), 9.15 (s, 1H), 8.61 (dd, 1H), 7.71 (s, 1H), 7.38 (dd, 1H), 6.82 (s, 1H), 6.40 (t, 1H), 5.05 (quin, 1H), 4.05 (dd, 1H), 3.93 (d, 1H), 3.68 (s, 3H), 2.6-2.7 (m, 4H), 1.8-2.3 (m, 8H), 1.48 (s, 3H). |
| | RCO₂H—C; RNH₂: 3-amino-1-methylpyridin-2(1H)-one | |
| 423 | 7-Ethoxy-N-(1-methyl-1H-pyrazol-3-yl)-2-(1-methyl-2-oxabicyclo[2.2.2]octan- | 25 mg, 58% |
| | 4-yl)imidazo[1,2-a]pyrimidine-6-carboxamide | LCMS m/z = 411.2 [M + H]⁺ |
| | | ¹H NMR (400 MHz, MeOH- |
| | 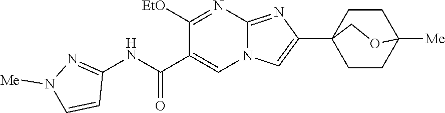 [SMILES: CCOc1nc2nc(C34CCC(C)(CC3)OC4)cn2cc1C(=O)Nc1ccn(C)n1] | d₄) δ: 9.30 (s, 1H), 7.4-7.7 (m, 2H), 6.70 (d, 1H), 4.75 (q, 2H), 4.06 (s, 2H), 3.86 (s, 3H), 2.1-2.3 (m, 2H), 1.8-2.0 (m, 6H), 1.59 (t, 3H), 1.16 (s, 3H). |
| | RCO₂H—E; RNH₂: 1-methyl-1H-pyrazol-3-amine | |
| 424 | 7-Cyclobutoxy-N-(1-methyl-2-oxo-1,2-dihydropyridin-3-yl)-2-(1-methyl-2- | 39 mg, 66% |
| | oxabicyclo[2.1.1]hexan-4-yl)imidazo[1,2-a]pyridine-6-carboxamide | LCMS m/z = 435.3 [M + H]⁺ |
| | | ¹H NMR (400 MHz, MeOH- |
| | 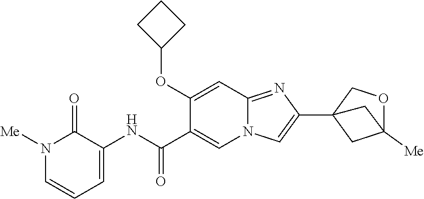 [SMILES: Cn1cccc(NC(=O)c2cn3cc(C45COC(C)(C4)C5)nc3cc2OC2CCC2)c1=O] | d₄) δ: 10.78 (s, 1H), 9.21 (s, 1H), 8.61 (dd, 1H), 7.80 (s, 1H), 7.39 (dd, 1H), 6.88 (s, 1H), 6.3-6.5 (m, 1H), 5.09 (t, 1H), 4.03 (s, 2H), 3.68 (s, 3H), 2.5-2.8 (m, 4H), 1.8-2.2 (m, 5H), 1.53 (s, 3H). |
| | RCO₂H—F; RNH₂: 3-amino-1-methylpyridin-2(1H)-one | |
| 425 | 7-Cyclobutoxy-2-(1-methyl-2-oxabicyclo[2.2.1]heptan-4-yl)-N- | 80 mg, 87% |
| | (pyrazolo[1,5-a]pyrimidin-3-yl)imidazo[1,2-a]pyridine-6-carboxamide | LCMS m/z = 459.3 [M + H]⁺ |
| | | ¹H NMR (400 MHz, MeOH- |
| | 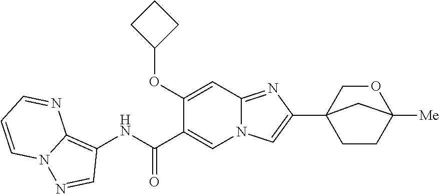 [SMILES: CC12CCC(c3cn4cc(C(=O)Nc5cnn6cccnc56)c(OC5CCC5)cc4n3)(CO1)C2] | d₄) δ: 9.15 (s, 1H), 8.87 (dd, 1H), 8.81 (s, 1H), 8.53 (dd, 1H), 7.71 (s, 1H), 7.03 (dd, 1H), 6.83 (s, 1H), 5.10 (quin, 1H), 4.07 (dd, 1H), 3.94 (d, 1H), 2.54-2.79 (m, 4H), 1.78- 2.26 (m, 8H), 1.48 (s, 3H). |
| | RCO₂H—C; RNH₂: pyrazolo[1,5-a]pyrimidin-3-amine | |
| 426 | 7-Cyclobutoxy-2-(1-methyl-2-oxabicyclo[2.2.1]heptan-4-yl)-N-(6- | 73 mg, 94% |
| | methylpyrazolo[1,5-a]pyrimidin-3-yl)imidazo[1,2-a]pyridine-6-carboxamide | LCMS m/z = 473.3 [M + H]⁺ |
| | | ¹H NMR (400 MHz, MeOH- |
| | 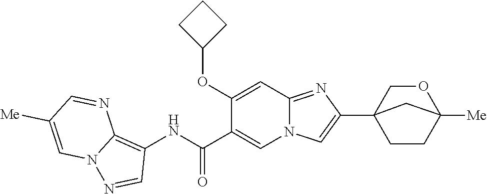 [SMILES: Cc1cnc2c(NC(=O)c3cn4cc(C56CCC(C)(C5)OC6)nc4cc3OC3CCC3)cnn2c1] | d₄) δ: 9.11 (s, 1H), 8.70 (s, 1H), 8.64 (dd, 1H), 8.43 (d, 1H), 7.70 (s, 1H), 6.80 (s, 1H), 5.08 (t, H), 4.06 (dd, H), 3.94 (d, 1H), 2.5-2.8 (m, 4H), 2.40 (d, 3H), 2.1-2.3 (m, 3H), 1.8-2.0 (m, 5H), 1.48 (s, 3H). |
| | RCO₂H—C; RNH₂: 6-methylpyrazolo[1,5-a]pyrimidin-3-amine | |
| 427 | 8-Isopropoxy-N-(1-methyl-2-oxo-1,2-dihydropyridin-3-yl)-2-(1-methyl-2- | 22 mg, 45% |
| | oxabicyclo[2.2.2]octan-4-yl)imidazo[1,2-a]pyrazine-6-carboxamide | LCMS m/z = 452.3 [M + H]⁺ |
| | | ¹H NMR (400 MHz, MeOH- |
| | 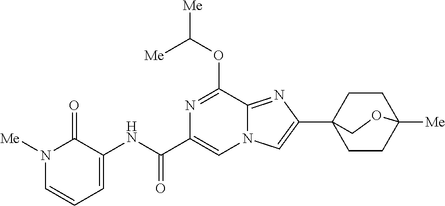 [SMILES: CC(C)Oc1nc(C(=O)Nc2cccn(C)c2=O)cn2cc(C34CCC(C)(CC3)OC4)nc12] | d₄) δ: 8.79 (s, 1H), 8.55 (dd, 1H), 7.90 (s, 1H), 7.40 (dd, 1H), 6.4-6.5 (m, 1H), 5.77 (quin, 1H), 4.11 (t, 2H), 3.69 (s, 3H), 1.7-2.3 (m, 8H), 1.60 (d, 6H), 1.17 (s, 3H). |
| | RCO₂H—G; RNH₂: 3-amino-1-methylpyridin-2(1H)-one | |
| 428 | 8-Isopropoxy-2-(1-methyl-2-oxabicyclo[2.2.2]octan-4-yl)-N-(pyrazolo[1,5- | 36 mg, 72% |
| | a]pyrimidin-3-yl)imidazo[1,2-a]pyrazine-6-carboxamide | LCMS m/z = 462.3 [M + H]⁺ |
| | | ¹H NMR (400 MHz, MeOH- |
| | 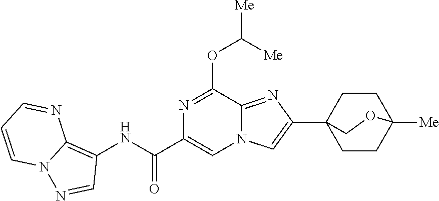 [SMILES: CC(C)Oc1nc(C(=O)Nc2cnn3cccnc23)cn2cc(C34CCC(C)(CC3)OC4)nc12] | d₄) δ: 8.91 (dd, 1H), 8.83 (s, 1H), 8.74 (s, 1H), 8.56 (dd, 1H), 7.92 (s, 1H), 7.05 (dd, 1H), 5.88 (quin, 1H), 4.12 (t, 2H), 1.8-2.3 (m, 8H), 1.59 (d, 6H), 1.17 (s, 3H). |
| | RCO₂H—G; RNH₂: pyrazolo[1,5-a]pyrimidin-3-amine | |
| 429 | 8-Isopropoxy-N-(1-methyl-1H-pyrazol-3-yl)-2-(1-methyl-2- | 8 mg, 17% |
| | oxabicyclo[2.2.2]octan-4-yl)imidazo[1,2-a]pyrazine-6-carboxamide | LCMS m/z = 425.3 [M + H]⁺ |
| | | ¹H NMR (400 MHz, MeOH- |
| | 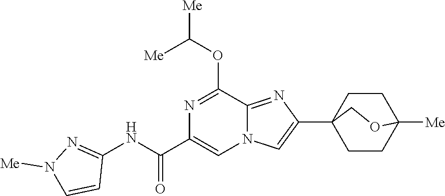 [SMILES: CC(C)Oc1nc(C(=O)Nc2ccn(C)n2)cn2cc(C34CCC(C)(CC3)OC4)nc12] | d₄) δ: 8.80 (s, 1H), 7.90 (s, 1H), 7.56 (d, 1H), 6.73 (d, 1H), 5.84 (td, 1H), 4.11 (t, 2H), 3.87 (s, 3H), 1.8-2.3 (m, 8H), 1.57 (d, 6H), 1.17 (s, 3H). |
| | RCO₂H—G; RNH₂: 1-methyl-1H-pyrazol-3-amine | |

### Example 430: 2-(2-Oxabicyclo[2.1.1]hexan-4-yl)-7-isopropoxy-N-(pyrazolo[1,5-a]pyrimidin-3-yl)imidazo[1,2-a]pyrimidine-6-carboxamide

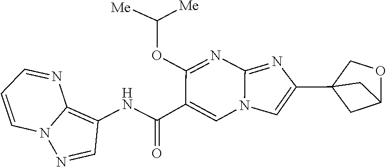

A mixture of 2-bromo-1-(2-oxabicyclo[2.1.1]hexan-4-yl)ethanone (15.6 mg, 0.076 mmol), 2-amino-4-isopropoxy-N-(pyrazolo[1,5-a]pyrimidin-3-yl)pyrimidine-5-carboxamide (Preparation 373, 14.8 mg, 0.047 mmol) and NaHCO₃ (12.8 mg, 0.152 mmol,) in MeCN (0.6 mL) and Toluene (0.4 mL) was heated at 90° C. overnight. The reaction mixture was partitioned between EtOAc and brine. The aqueous layer was extracted with EtOAc and the combined organics were dried and evaporated to dryness in vacuo. The residue was purified by SiO₂ column chromatography (12 g, EtOAc 100%) to afford 2-(2-oxabicyclo[2.1.1]hexan-4-yl)-7-isopropoxy-N-(pyrazolo[1,5-a]pyrimidin-3-yl)imidazo[1,2-a]pyrimidine-6-carboxamide as a yellow solid (5.8 mg, 29%). LCMS m/z=420.2 [M+H]⁺; ¹H NMR (400 MHz, MeOH-d₄) δ: 9.40 (s, 1H), 8.85 (dd, 1H), 8.78 (s, 1H), 8.52 (dd, 1H), 7.67 (s, 1H), 7.01 (dd, 1H), 5.82-5.66 (m, 1H), 4.68-4.63 (m, 1H), 3.96 (s, 2H), 2.29-2.23 (m, 2H), 1.91-1.84 (m, 2H), 1.66 (d, 6H).

### Example 431-434

The title compounds were prepared from the appropriate aminopyrimidine (R—NH₂) and appropriate halide (R-Hal) using an analogous method to that described for Example 430.

| Ex. No | Name/Structure/R-NH₂/R-Hal | Data |
|---|---|---|
| 431 | 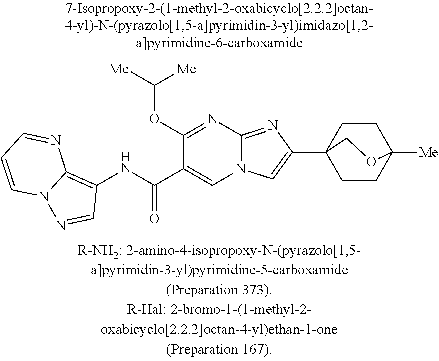 [SMILES: CC(C)Oc1nc2nc(C34CCC(C)(CC3)OC4)cn2cc1C(=O)Nc1cnn2cccnc12] | 7.8 mg, 33% LCMS m/z = 462.2 [M + H]⁺; ¹H NMR (400 MHz, MeOH-d₄) δ: 9.40 (s, 1H), 8.87 (dd, 1H), 8.80 (s, 1H), 8.54 (dd, 1H), 7.55 (s, 1H), 7.04 (dd, 1H), 5.75 (quin, 1H), 4.07 (s, 2H), 2.25- 1.78 (m, 9H), 1.67 (d, 6H), 1.17 (s, 3H). |
| 432 | 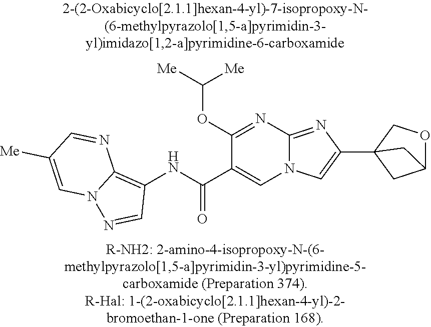 | 13 mg, 51% LCMS m/z = 434.2 [M + H]⁺ ¹H NMR (400 MHz, MeOH-d₄) δ: 9.42 (s, 1H), 8.71 (s, 1H), 8.68 (dd, 1H), 8.47 (d, 1H), 7.69 (s, 1H), 5.76 (quin, 1H), 4.67 (s, 1H), 3.99 (s, 2H), 2.43 (d, 3H), 2.28 (d, 2H), 1.88-1.92 (m, 2H), 1.68 (d, 6H). |
| 433 | 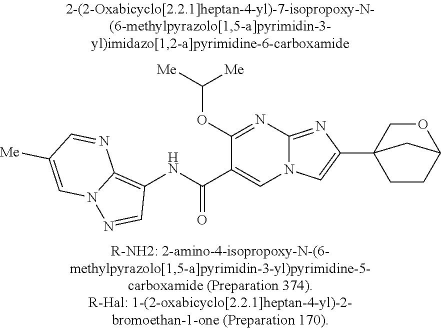 | 8 mg, 16% LCMS m/z = 448.2 [M + H]⁺; ¹H NMR (400 MHz, MeOH-d₄) δ: 9.41 (s, 1H), 8.71 (s, 1H), 8.69 (dd, 1H), 8.47 (d, 1H), 7.66 (s, 1H), 5.76 (quin, 1H), 4.52 (d, 1H), 3.96 (dd, 1H), 3.87 (d, 1H), 3.3-3.4 (m, 1H), 2.43 (d, 3H), 1.8-2.2 (m, 6H), 1.68 (d, 6H). |
| 434 | 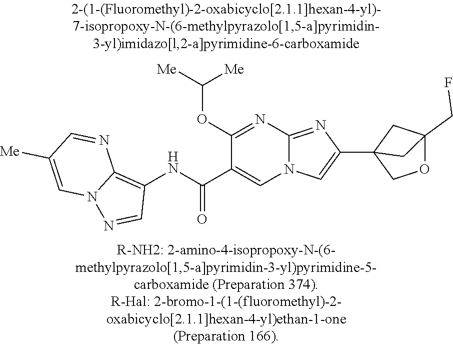 [SMILES: Cc1cnc2c(NC(=O)c3cn4cc(C56COC(CF)(C5)C6)nc4nc3OC(C)C)cnn2c1] | 46 mg, 25% LCMS m/z = 466.2 [M + H]⁺ ¹H NMR (400 MHz, MeOH-d₄) δ: 9.60 (s, 1H), 8.73 (s, 1H), 8.72 (s, 1H), 8.49 (d, 1H), 8.00 (s, 1H), 5.74-5.84 (m, 1H), 4.77 (s, 1H), 4.65 (s, 1H), 4.13 (s, 2H), 2.44 (s, 3H), 2.42- 2.46 (m, 1H), 2.34- 2.40 (m, 2H), 2.09-2.14 (m, 2H), 1.71 (d, 6H). |

### Example 435: N-(6-methoxypyridin-2-yl)-2-(tetrahydro-2H-pyran-4-yl)-7-(2-(tetrahydrofuran-3-yl)ethoxy)imidazo[1,2-a]pyridine-6-carboxamide

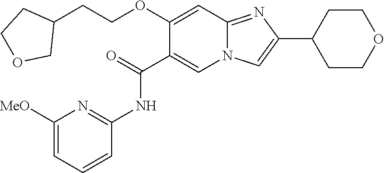 [SMILES: COc1cccc(NC(=O)c2cn3cc(C4CCOCC4)nc3cc2OCCC2CCOC2)n1]

K₂CO₃ (37.8 mg, 0.27 mmol) and NaI (7.1 mg, 0.05 mmol) were added 7-hydroxy-N-(6-methoxypyridin-2-yl)-2-(tetrahydro-2H-pyran-4-yl)imidazo[1,2-a]pyridine-6-carboxamide (Preparation 385, 39.5 mg, 0.09 mmol) and 3-(2-bromoethyl)tetrahydrofuran (28.8 mg, 0.16 mmol) were dissolved in dry DMF (1 mL) and the resulting mixture was heated with stirring for 8 h at 90° C. The reaction was evaporated to dryness in vacuo and the residue dissolved in DMSO, acidified with acetic acid and purified by prep-HPLC-E (gradient: 40-90%) to afford N-(6-methoxypyridin-2-yl)-2-(tetrahydro-2H-pyran-4-yl)-7-(2-(tetrahydrofuran-3-yl)ethoxy)imidazo[1,2-a]pyridine-6-carboxamide (26.7 mg, 53%). LCMS m/z=467.3 [M+H]t

### Example 436: N-(6-(difluoromethyl)pyridin-2-yl)-7-((1s,3s)-3-ethoxycyclobutoxy)-2-(tetrahydro-2H-pyran-4-yl)imidazo[1,2-a]pyridine-6-carboxamide

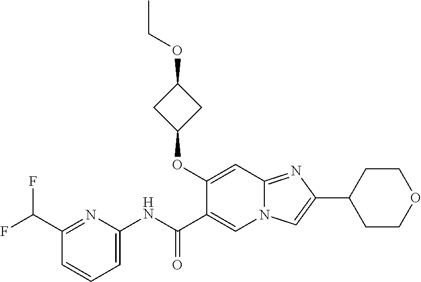 [SMILES: CCO[C@H]1C[C@@H](Oc2cc3nc(C4CCOCC4)cn3cc2C(=O)Nc2cccc(C(F)F)n2)C1]

K₂CO₃ (42.6 mg, 0.31 mmol) was added to N-(6-(difluoromethyl)pyridin-2-yl)-7-hydroxy-2-(tetrahydro-2H-pyran-4-yl)imidazo[1,2-a]pyridine-6-carboxamide (Preparation 386, 39.9 mg, 0.10 mmol) and (1r,3r)-1-bromo-3-ethoxycyclobutane (27.6 mg, 0.15 mmol) were dissolved in dry DMF (1 mL) and the resulting mixture was heated with stirring for 8 h at 90° C. The reaction was evaporated to dryness in vacuo and the residue dissolved in DMSO, acidified with acetic acid and purified by prep-HPLC-E (gradient: 40-90%) to afford N-(6-(difluoromethyl)pyridin-2-yl)-7-((1s,3 s)-3-ethoxycyclobutoxy)-2-(tetrahydro-2H-pyran-4-yl)imidazo[1,2-a]pyridine-6-carboxamide (11.4 mg, 22%). LCMS m/z=487.0 [M+H]t

### Example 437-47

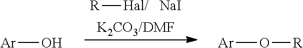

All the syntheses were performed on a 100 mg product scale. The title compounds were prepared on a 100 mg product scale using a single step library protocol. K₂CO₃ (3.0 equiv.) and NaI (0.5 equiv.) were added to the appropriate scaffold (ArOH, 1 equiv.) and alkyl halide (R-Hal, 1.2 equiv.) in DMF and the resulting mixture stirred at 90° C. for 8 h. The reaction mixture was evaporated to dryness in vacuo and the residue dissolved in DMSO, acidified with acetic acid and purified by prep-HPLC-G (gradient 0-100% using a gradient optimised for each compound to afford the title compounds.

ArOH-1: 7-hydroxy-N-(1-methyl-1H-pyrazol-3-yl)-2-(1-methyl-2-oxabicyclo[2.1.1]hexan-4-yl)imidazo[1,2-a]pyridine-6-carboxamide (Preparation 387); ArOH-2: N-(1-(difluoromethyl)-2-oxo-1,2-dihydropyridin-3-yl)-7-hydroxy-2-(1-methyl-2-oxabicyclo[2.1.1]hexan-4-yl)imidazo[1,2-a]pyridine-6-carboxamide (Preparation 388); ArOH-3: 2-cyclopropyl-7-hydroxy-N-(2-methoxypyridin-3-yl)imidazo[1,2-a]pyridine-6-carboxamide (Preparation 389); ArOH-4: 2-cyclopropyl-7-hydroxy-N-(6-methoxypyridin-2-yl)imidazo[1,2-a]pyridine-6-carboxamide (Preparation 390); ArOH-5: 2-cyclopropyl-N-(6-(difluoromethyl)pyridin-2-yl)-7-hydroxyimidazo[1,2-a]pyridine-6-carboxamide (Preparation 391); ArOH-6: 2-cyclopropyl-N-(1-(difluoromethyl)-2-oxo-1,2-dihydropyridin-3-yl)-7-hydroxyimidazo[1,2-a]pyridine-6-carboxamide (Preparation 393)

| Ex. | | |
|---|---|---|
| No. | Name/Structure/Reactants | Yield/Data |
| 437 | 7-Cyclobutoxy-N-(1-methyl-1H-pyrazol-3- yl)-2-(1-methyl-2-oxabicyclo[2.1.1]hexan-4- yl)imidazo[1,2-a]pyridine-6-carboxamide 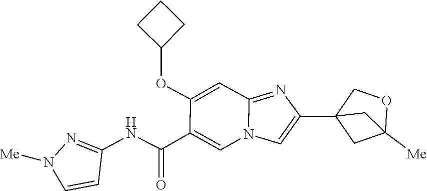 [SMILES: Cn1ccc(NC(=O)c2cn3cc(C45COC(C)(C4)C5)nc3cc2OC2CCC2)n1] ArOH-1. RHal: bromocyclobutane | 9.7 mg, 9.7% LCMS m/z = 408.2 [M + H]⁺ |
| 438 | N-(1-(difluoromethyl)-2-oxo-1,2- dihydropyridin-3-yl)-7-(2-fluoroethoxy)-2-(1- methyl-2-oxabicyclo[2.1.1]hexan-4- yl)imidazo[1,2-a]pyridine-6-carboxamide 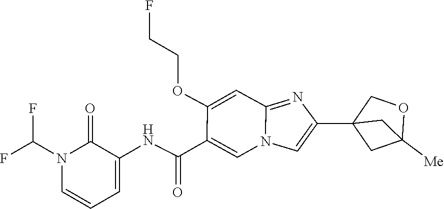 [SMILES: CC12CC(c3cn4cc(C(=O)Nc5cccn(C(F)F)c5=O)c(OCCF)cc4n3)(CO1)C2] ArOH-2. RHal: 1-bromo-2-fluoroethane | Yield: 4.9 mg, 4.9% LCMS m/z = 463.0 [M + H]⁺ |
| 439 | N-(1-(difluoromethyl)-2-oxo-1,2- dihydropyridin-3-yl)-7-(2,2- dimethylcyclobutoxy)-2-(1-methyl-2- oxabicyclo[2.1.1]hexan-4-yl)imidazo[1,2- a]pyridine-6-carboxamide 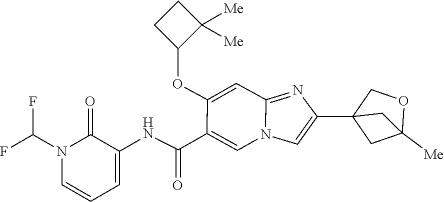 [SMILES: CC12CC(c3cn4cc(C(=O)Nc5cccn(C(F)F)c5=O)c(OC5CCC5(C)C)cc4n3)(CO1)C2] ArOH-2 RHal: 2-bromo-1,1-dimethylcyclobutane | Yield: 9.4 mg, 9.4% LCMS m/z = 499.2 [M + H]⁺ ¹H NMR (500 MHz, DMSO- d₆) δ: 10.60 (s, 1H), 9.18 (s, 1H), 8.51 (d, 1H), 7.98 (t, 1H), 7.83 (s, 1H), 7.57 (d, 1H), 7.15 (s, 1H), 6.54 (t, 1H), 5.23 (t, 1H), 4.24 (t, 2H), 3.87 (s, 2H), 2.76-2.65 (m, 2H), 2.03-1.94 (m, 2H), 1.79-1.71 (m, 2H), 1.63 (s, 3H), 1.58 (s, 3H), 1.42 (s, 3H). |
| 440 | 7-(3,3-Dimethylcyclobutoxy)-N-(1-methyl- 1H-pyrazol-3-yl)-2-(1-methyl-2- oxabicyclo[2.1.1]hexan-4-yl)imidazo[1,2- a]pyridine-6-carboxamide 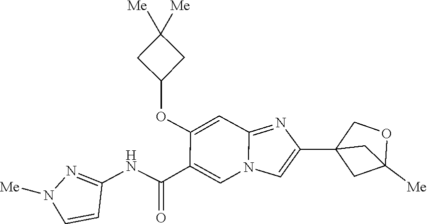 [SMILES: Cn1ccc(NC(=O)c2cn3cc(C45COC(C)(C4)C5)nc3cc2OC2CC(C)(C)C2)n1] ArOH-1 RHal: 3-bromo-1,1-dimethylcyclobutane | Yield: 5.6 mg, 5.6% LCMS m/z = 436.2 [M + H]+ |
| 441 | N-(1-(difluoromethyl)-2-oxo-1,2- dihydropyridin-3-yl)-2-(1-methyl-2- oxabicyclo[2.1.1]hexan-4-yl)-7-(oxetan-3- yloxy)imidazo[1,2-a]pyridine-6-carboxamide 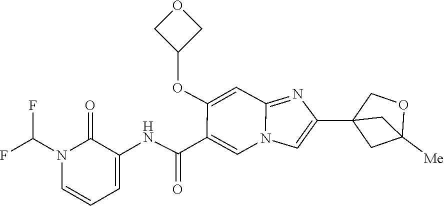 [SMILES: CC12CC(c3cn4cc(C(=O)Nc5cccn(C(F)F)c5=O)c(OC5COC5)cc4n3)(CO1)C2] ArOH-2. RHal: 3-bromooxetane | Yield: 7.9 mg, 7.9% LCMS m/z = 473.1 [M + H]+ |
| 442 | 7-Cyclobutoxy-N-(1-(difluoromethyl)-2-oxo- 1,2-dihydropyridin-3-yl)-2-(1-methyl-2- oxabicyclo[2.1.1]hexan-4-yl)imidazo[1,2- a]pyridine-6-carboxamide 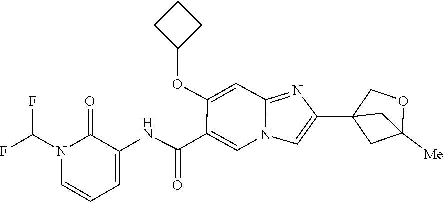 [SMILES: CC12CC(c3cn4cc(C(=O)Nc5cccn(C(F)F)c5=O)c(OC5CCC5)cc4n3)(CO1)C2] ArOH-2. RHal: bromocyclobutane | Yield: 7.3 mg, 7.3% LCMS m/z = 471.2 [M + H]+ |
| 443 | N-(1-(difluoromethyl)-2-oxo-1,2- dihydropyridin-3-yl)-2-(1-methyl-2- oxabicyclo[2.1.1]hexan-4-yl)-7- ((tetrahydrofuran-3-yl)oxy)imidazo[1,2- a]pyridine-6-carboxamide 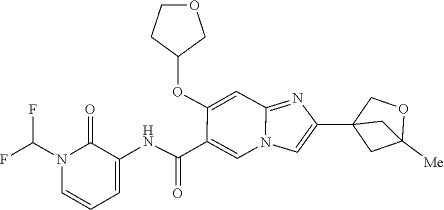 [SMILES: CC12CC(c3cn4cc(C(=O)Nc5cccn(C(F)F)c5=O)c(OC5CCOC5)cc4n3)(CO1)C2] ArOH-2. RHal: 3-bromotetrahydrofuran | Yield: 6.8 mg, 6.8% LCMS m/z = 487.2 [M + H]+ |
| 444 | N-(1-(difluoromethyl)-2-oxo-1,2- dihydropyridin-3-yl)-2-(1-methyl-2- oxabicyclo[2.1.1]hexan-4-yl)-7-((3- methyltetrahydrofuran-3- yl)methoxy)imidazo[1,2-a]pyridine-6- carboxamide 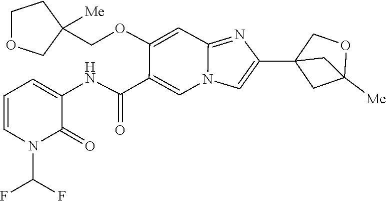 [SMILES: CC1(COc2cc3nc(C45COC(C)(C4)C5)cn3cc2C(=O)Nc2cccn(C(F)F)c2=O)CCOC1] ArOH-2. RHal: 3-(bromomethyl)-3- methyltetrahydrofuran | Amount: 8.3 mg, 8.3% LCMS m/z = 515.2 [M + H]+ ¹H NMR (500 MHz, DMSO-d₆) δ: 10.00 (s, 1H), 9.13 (s, 1H), 8.55-8.45 (m, 1H), 7.96 (t, 1H), 7.82 (s, 1H), 7.63-7.55 (m, 1H), 7.21 (s, 1H), 6.55 (t, 1H), 4.17-4.05 (m, 2H), 3.87 (s, 2H), 3.81-3.79 (m, 1H), 3.77-3.71 (m, 1H), 3.47 (d, 1H), 2.07 (s, 2H), 2.02-1.97 (m, 2H), 1.97-1.92 (m, 1H), 1.76-1.74 (m, 2H), 1.42 (s, 3H), 1.25 (s, 3H). |
| 445 | N-(1-(difluoromethyl)-2-oxo-1,2- dihydropyridin-3-yl)-7-(3,3- dimethylcyclobutoxy)-2-(1-methyl-2- oxabicyclo[2.1.1]hexan-4-yl)imidazo[1,2- a]pyridine-6-carboxamide 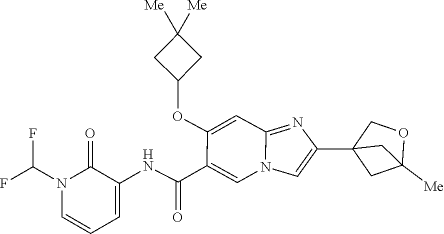 [SMILES: CC1(C)CC(Oc2cc3nc(C45COC(C)(C4)C5)cn3cc2C(=O)Nc2cccn(C(F)F)c2=O)C1] ArOH-2 RHal: 3-bromo-1,1-dimethylcyclobutane | Yield: 15.5 mg, 15.5% LCMS m/z = 499.2 [M + H]+ ¹H NMR (400 MHz, CDCl₃) δ: 10.79 (s, 1H), 8.98 (s, 1H), 8.61 (d, 1H), 7.80 (t, 1H), 7.35 (s, 1H), 7.24 (d, 1H), 6.85 (s, 1H), 6.40 (t, 1H), 4.93-4.80 (m, 1H), 4.06 (s, 2H), 2.54- 2.43 (m, 2H), 2.43-2.33 (m, 2H), 2.13-2.03 (m, 2H), 2.01- 1.91 (m, 2H), 1.54 (s, 3H), 1.26 (s, 4H), 1.23 (s, 3H). |
| 446 | 7-(2-(Difluoromethoxy)ethoxy)-N-(1- (difluoromethyl)-2-oxo-1,2-dihydropyridin-3- yl)-2-(1-methyl-2-oxabicyclo[2.1.1]hexan-4- yl)imidazo[1,2-a]pyridine-6-carboxamide 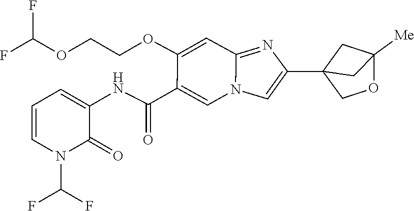 [SMILES: CC12CC(c3cn4cc(C(=O)Nc5cccn(C(F)F)c5=O)c(OCCOC(F)F)cc4n3)(CO1)C2] ArOH-2 RHal: 1-bromo-2-(difluoromethoxy)ethane | Yield: 16.2 mg, 16.2% LCMS m/z = 511.2 [M + H]+ ¹H NMR (400 MHz, CDCl₃) δ: 10.62 (s, 1H), 8.99 (s, 1H), 8.63-8.52 (m, 1H), 7.73 (t, 1H), 7.39 (s, 1H), 7.26-7.22 (m, 1H), 7.00 (s, 1H), 6.57-6.11 (m, 2H), 4.60-4.50 (m, 2H), 4.46-4.38 (m, 2H), 4.06 (s, 2H), 2.09- 2.05 (m, 2H), 1.99-1.93 (m, 2H), 1.54 (s, 3H). |
| 447 | N-(1-(difluoromethyl)-2-oxo-1,2- dihydropyridin-3-yl)-7-((5,5- dimethyltetrahydrofuran-3-yl)oxy)-2-(1- methyl-2-oxabicyclo[2.1.1]hexan-4- yl)imidazo[1,2-a]pyridine-6-carboxamide 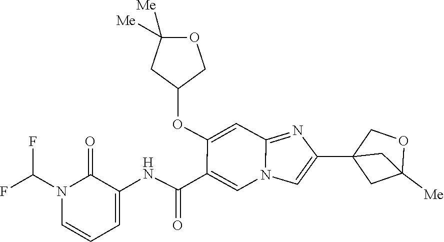 [SMILES: CC1(C)CC(Oc2cc3nc(C45COC(C)(C4)C5)cn3cc2C(=O)Nc2cccn(C(F)F)c2=O)CO1] ArOH-2 RHal: 4-bromo-2,2-dimethyltetrahydrofuran | Yield: 11.8 mg, 11.8% LCMS m/z = 515.1 [M + H]+ ¹H NMR (400 MHz, CDCl₃) δ: 10.53 (s, 1H), 9.00 (s, 1H), 8.71-8.54 (m, 1H), 7.78 (t, 1H), 7.37 (s, 1H), 7.27-7.21 (m, 1H), 6.85 (s, 1H), 6.41 (t, 1H), 5.24-5.06 (m, 1H), 4.57- 4.47 (m, 1H), 4.39-4.27 (m, 1H), 4.06 (s, 2H), 2.61-2.47 (m, 1H), 2.39-2.23 (m, 1H), 2.11-2.04 (m, 2H), 1.99-1.94 (m, 2H), 1.54 (s, 3H), 1.37 (s, 3H), 1.33 (s, 3H). |
| 448 | 7-((3,3-Difluorocyclobutyl)methoxy)-N-(1- (difluoromethyl)-2-oxo-1,2-dihydropyridin-3- yl)-2-(1-methyl-2-oxabicyclo[2.1.1]hexan-4- yl)imidazo[1,2-a]pyridine-6-carboxamide 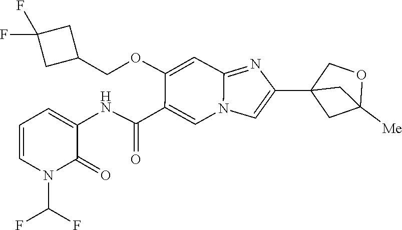 [SMILES: CC12CC(c3cn4cc(C(=O)Nc5cccn(C(F)F)c5=O)c(OCC5CC(F)(F)C5)cc4n3)(CO1)C2] ArOH-2 RHal: 3-(bromomethyl)-1,1-difluorocyclobutane | Yield: 19.1 mg, 19.1% LCMS m/z = 521.1 [M + H]+ ¹H NMR (400 MHz, DMSO- d₆ + CCl₄) δ: 10.51 (s, 1H), 9.18 (s, 1H), 8.56 (d, 1H), 7.88 (t, 1H), 7.69 (s, 1H), 7.40 (d, 1H), 7.11 (s, 1H), 6.49 (t, 1H), 4.34 (d, 2H), 3.88 (s, 2H), 3.06-3.01 (m, 2H), 2.95-2.81 (m, 2H), 2.48-2.41 (m, 1H), 2.03-1.93 (m, 2H), 1.83-1.74 (m, 2H), 1.45 (s, 3H). |
| 449 | 7-(Cyclopentyloxy)-N-(1-methyl-1H-pyrazol- 3-yl)-2-(1-methyl-2-oxabicyclo[2.1.1]hexan- 4-yl)imidazo[1,2-a]pyridine-6-carboxamide 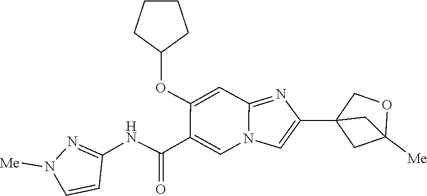 [SMILES: Cn1ccc(NC(=O)c2cn3cc(C45COC(C)(C4)C5)nc3cc2OC2CCCC2)n1] ArOH-1 RHal: bromocyclopentane | Yield: 11 mg, 11% LCMS m/z = 422.2 [M + H]+ ¹H NMR (400 MHz, CDCl₃) δ: 10.20 (s, 1H), 9.01 (s, 1H), 7.35 (s, 1H), 7.28 (d, 1H), 6.97 (s, 1H), 6.75 (d, 1H), 5.05- 4.96 (m, 1H), 4.06 (s, 2H), 3.83 (s, 3H), 2.14-2.06 (m, 6H), 2.00-1.91 (m, 4H), 1.82- 1.72 (m, 2H), 1.53 (s, 3H). |
| 450 | N-(1-(difluoromethyl)-2-oxo-1,2- dihydropyridin-3-yl)-7-(3- (difluoromethyl)cyclobutoxy)-2-(1-methyl-2- oxabicyclo[2.1.1]hexan-4-yl)imidazo[1,2- a]pyridine-6-carboxamide 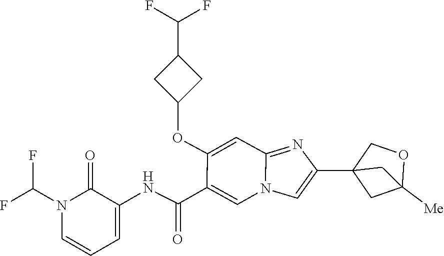 [SMILES: CC12CC(c3cn4cc(C(=O)Nc5cccn(C(F)F)c5=O)c(OC5CC(C(F)F)C5)cc4n3)(CO1)C2] ArOH-2 RHal: 1-bromo-3- (difluoromethyl)cyclobutane | Yield: 16 mg, 16% LCMS m/z = 521.2 [M + H]+ ¹H NMR (400 MHz, CDCl₃) δ: 10.79-10.68 (m, 1H), 9.00 (s, 1H), 8.67-8.53 (m, 1H), 7.99-7.58 (m, 1H), 7.37 (s, 1H), 7.26-7.22 (m, 1H), 6.87- 6.74 (m, 1H), 6.48-6.34 (m, 1H), 6.18-5.71 (m, 1H), 5.05- 4.79 (m, 1H), 4.06 (s, 2H), 2.93-2.79 (m, 1H), 2.79-2.64 (m, 3H), 2.64-2.50 (m, 1H), 2.11-2.02 (m, 2H), 2.00-1.91 (m, 2H), 1.54 (s, 3H). |
| 451 | 7-(3-(Difluoromethyl)cyclobutoxy)-N-(1- methyl-1H-pyrazol-3-yl)-2-(1-methyl-2- oxabicyclo[2.1.1]hexan-4-yl)imidazo[1,2- a]pyridine-6-carboxamide 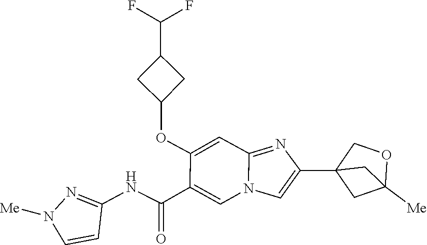 [SMILES: Cn1ccc(NC(=O)c2cn3cc(C45COC(C)(C4)C5)nc3cc2OC2CC(C(F)F)C2)n1] ArOH-1 RHal: 1-bromo-3- (difluoromethyl)cyclobutane | Yield: 13.4 mg, 13.4% LCMS m/z = 458.2 [M + H]+ ¹H NMR (400 MHz, CDCl₃) δ: 10.10-9.90 (m, 1H), 9.02 (s, 1H), 7.37 (s, 1H), 7.30 (d, 1H), 6.82-6.72 (m, 2H), 6.16-5.70 (m, 1H), 5.04-4.75 (m, 1H), 4.06 (s, 2H), 3.84 (s, 3H), 3.08-2.47 (m, 4H), 2.46-2.35 (m, 1H), 2.10-2.04 (m, 2H), 1.98-1.92 (m, 2H), 1.53 (s, 3H) |
| 452 | 7-((2,2-Difluorocyclopropyl)methoxy)-N-(1- (difluoromethyl)-2-oxo-1,2-dihydropyridin-3- yl)-2-(1-methyl-2-oxabicyclo[2.1.1]hexan-4- yl)imidazo[1,2-a]pyridine-6-carboxamide 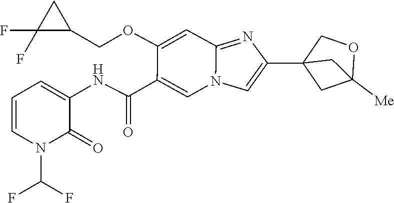 [SMILES: CC12CC(c3cn4cc(C(=O)Nc5cccn(C(F)F)c5=O)c(OCC5CC5(F)F)cc4n3)(CO1)C2] ArOH-2 RHal: 2-(bromomethyl)-1,1- difluorocyclopropane | Yield: 11.4 mg, 11.4% LCMS m/z = 507.2 [M + H]+ ¹H NMR (400 MHz, DMSO- d₆ + CCl₄) δ: 10.67 (s, 1H), 9.20 (s, 1H), 8.55 (d, 1H), 8.05-7.66 (m, 2H), 7.39 (d, 1H), 7.12 (s, 1H), 6.48 (t, 1H), 4.55-4.45 (m, 1H), 4.32-4.20 (m, 1H), 3.88 (s, 2H), 2.09- 2.02 (m, 2H), 2.02-1.93 (m, 2H), 1.84-1.69 (m, 3H), 1.44 (s, 3H). |
| 453 | N-(1-(difluoromethyl)-2-oxo-1,2- dihydropyridin-3-yl)-7-isobutoxy-2-(1- methyl-2-oxabicyclo[2.1.1]hexan-4- yl)imidazo[1,2-a]pyridine-6-carboxamide 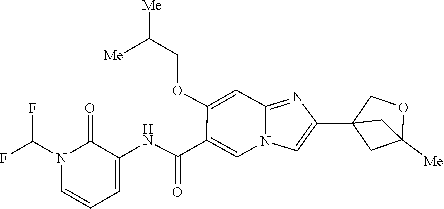 [SMILES: CC(C)COc1cc2nc(C34COC(C)(C3)C4)cn2cc1C(=O)Nc1cccn(C(F)F)c1=O] ArOH-2. RHal: 1-bromo-2-methylpropane | Yield: 5.7 mg, 5.7% LCMS m/z = 473.2 [M + H]+ |
| 454 | N-(1-(difluoromethyl)-2-oxo-1,2- dihydropyridin-3-yl)-2-(1-methyl-2- oxabicyclo[2.1.1]hexan-4-yl)-7-((tetrahydro- 2H-pyran-4-yl)oxy)imidazo[1,2-a]pyridine-6- carboxamide 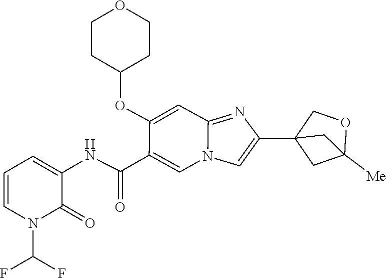 [SMILES: CC12CC(c3cn4cc(C(=O)Nc5cccn(C(F)F)c5=O)c(OC5CCOCC5)cc4n3)(CO1)C2] ArOH-2. RHal: 4-bromotetrahydro-2H-pyran | LCMS m/z = 501.1 [M + H]⁺ |
| 455 | 7-(3,3-Difluorocyclobutoxy)-N-(1- (difluoromethyl)-2-oxo-1,2-dihydropyridin-3- yl)-2-(1-methyl-2-oxabicyclo[2.1.1]hexan-4- yl)imidazo[1,2-a]pyridine-6-carboxamide 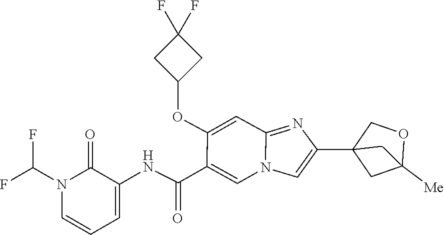 [SMILES: CC12CC(c3cn4cc(C(=O)Nc5cccn(C(F)F)c5=O)c(OC5CC(F)(F)C5)cc4n3)(CO1)C2] ArOH-2 RHal: 3-bromo-1,1-difluorocyclobutane | LCMS m/z = 507.2 [M + H]⁺ |
| 456 | N-(1-(difluoromethyl)-2-oxo-1,2- dihydropyridin-3-yl)-7-((4-fluorotetrahydro- 2H-pyran-4-yl)methoxy)-2-(1-methyl-2- oxabicyclo[2.1.1]hexan-4-yl)imidazo[1,2- a]pyridine-6-carboxamide 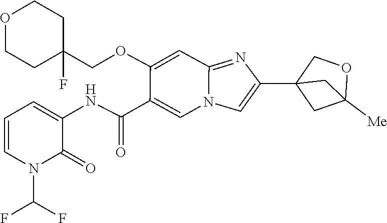 [SMILES: CC12CC(c3cn4cc(C(=O)Nc5cccn(C(F)F)c5=O)c(OCC5(F)CCOCC5)cc4n3)(CO1)C2] ArOH-2. RHal: 4-(bromomethyl)-4- fluorotetrahydro-2H-pyran | LCMS m/z = 533.0 [M + H]⁺ |
| 457 | 7-(Cyclopentyloxy)-N-(1-(difluoromethyl)-2- oxo-1,2-dihydropyridin-3-yl)-2-(1-methyl-2- oxabicyclo[2.1.1]hexan-4-yl)imidazo[1,2- a]pyridine-6-carboxamide 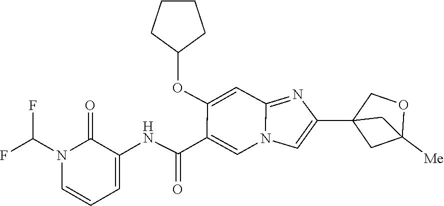 [SMILES: CC12CC(c3cn4cc(C(=O)Nc5cccn(C(F)F)c5=O)c(OC5CCCC5)cc4n3)(CO1)C2] ArOH-2 RHal: bromocyclopentane | Yield: 13 mg, 13% LCMS m/z = 485.0 [M + H]+ ¹H NMR (400 MHz, CDCl₃) δ: 10.62 (s, 1H), 8.98 (s, 1H), 8.62 (d, 1H), 7.79 (t, 1H), 7.36 (s, 1H), 7.24 (d, 1H), 7.01 (s, 1H), 6.40 (t, 1H), 5.07-4.94 (m, 1H), 4.08 (s, 2H), 2.30- 2.22 (m, 2H), 2.15-2.05 (m, 4H), 2.01-1.96 (m, 2H), 1.96- 1.86 (m, 2H), 1.74-1.64 (m, 2H), 1.54 (s, 3H). |
| 458 | 7-(Cyclopentyloxy)-2-cyclopropyl-N-(1- (difluoromethyl)-2-oxo-1,2-dihydropyridin-3- yl)imidazo[1,2-a]pyridine-6-carboxamide 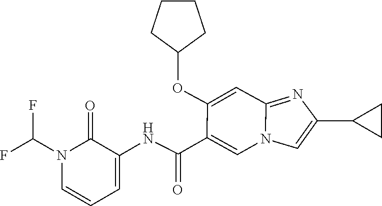 [SMILES: O=C(Nc1cccn(C(F)F)c1=O)c1cn2cc(C3CC3)nc2cc1OC1CCCC1] ArOH-6 RHal: bromocyclopentane | Yield: 12.1 mg, 15% LCMS m/z = 429.0 [M + H]⁺ ¹H NMR (600 MHz, DMSO-d₆) δ: 10.47 (s, 1H), 9.11 (s, 1H), 8.52 (d, 1H), 7.95 (t, 1H), 7.73 (s, 1H), 7.55 (d, 1H), 6.99 (s, 1H), 6.52 (t, 1H), 5.14-5.09 (m, 1H), 2.10-2.01 (m, 4H), 1.98-1.92 (m, 1H), 1.81-1.75 (m, 2H), 1.61-1.55 (m, 2H), 0.89-0.85 (m, 2H), 0.82-0.77 (m, 2H). |
| 459 | 2-Cyclopropyl-N-(1-(difluoromethyl)-2-oxo- 1,2-dihydropyridin-3-yl)-7-((tetrahydrofuran- 3-yl)oxy)imidazo[1,2-a]pyridine-6- carboxamide 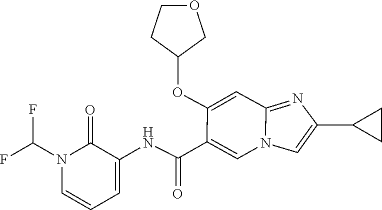 [SMILES: O=C(Nc1cccn(C(F)F)c1=O)c1cn2cc(C3CC3)nc2cc1OC1CCOC1] ArOH-6. RHal: 3-bromotetrahydrofuran | Yield: 6.3 mg, 7.9% LCMS m/z = 431.2 [M + H]⁺ |
| 460 | 2-Cyclopropyl-N-(1-(difluoromethyl)-2-oxo- 1,2-dihydropyridin-3-yl)-7-(3,3- dimethylcyclobutoxy)imidazo[1,2-a]pyridine- 6-carboxamide 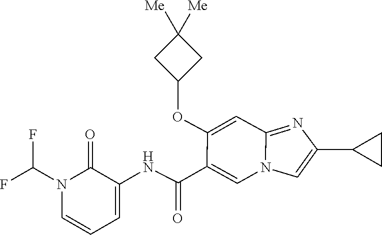 [SMILES: CC1(C)CC(Oc2cc3nc(C4CC4)cn3cc2C(=O)Nc2cccn(C(F)F)c2=O)C1] ArOH-6. RHal: 3-bromo-1,1- dimethylcyclobutane | Yield: 2 mg, 2.5% LCMS m/z = 443.2 [M + H]⁺ |
| 461 | 2-Cyclopropyl-N-(1-(difluoromethyl)-2-oxo- 1,2-dihydropyridin-3-yl)-7-(3- (difluoromethyl)cyclobutoxy)imidazo[1,2- a]pyridine-6-carboxamide 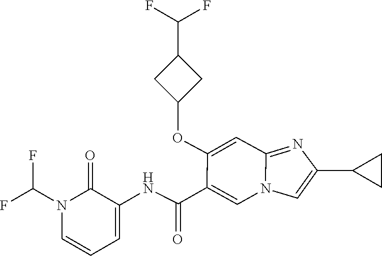 [SMILES: O=C(Nc1cccn(C(F)F)c1=O)c1cn2cc(C3CC3)nc2cc1OC1CC(C(F)F)C1] ArOH-6. RHal: 1-bromo-3- (difluoromethyl)cyclobutane | Yield: 4 mg, 5% LCMS m/z = 465.2 [M + H]⁺ |
| 462 | 7-Cyclobutoxy-2-cyclopropyl-N-(2- methoxypyridin-3-yl)imidazo[1,2-a]pyridine- 6-carboxamide 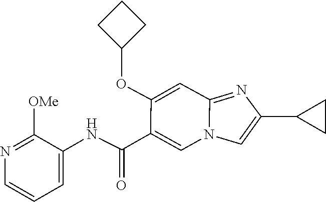 [SMILES: COc1ncccc1NC(=O)c1cn2cc(C3CC3)nc2cc1OC1CCC1] ArOH-3 RHal: bromocyclobutane | Amount: 11.8 mg, 19.7 mg LCMS m/z = 379.2; ¹H NMR (DMSO-d₆, 500 MHz) δ: 10.21 (s, 1H), 9.22 (s, 1H), 8.72 (d, 1H), 7.93 (d, 1H), 7.84 (s, 1H), 7.08-7.05 (m, 1H), 6.92 (s, 1H), 5.09 (p, 1H), 4.01 (s, 3H), 2.71-2.61 (m, 2H), 2.35-2.27 (m, 2H), 2.09- 2.01 (m, 1H), 1.96-1.90 (m, 1H), 1.83-1.73 (m, 1H), 0.99- 0.94 (m, 2H), 0.88-0.83(m, 2H) |
| 463 | 7-Cyclobutoxy-2-cyclopropyl-N-(6- methoxypyridin-2-yl)imidazo[1,2-a]pyridine- 6-carboxamide 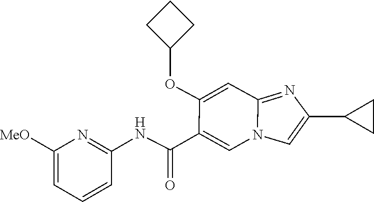 [SMILES: COc1cccc(NC(=O)c2cn3cc(C4CC4)nc3cc2OC2CCC2)n1] ArOH-4. RHal: bromocyclobutane | Amount: 6 mg, 10% LCMS m/z = 379.2; ¹H NMR(DMSO-d₆, 500 MHz) δ: |
| 464 | 7-(Cyclopentyloxy)-2-cyclopropyl-N-(2- methoxypyridin-3-yl)imidazo[1,2-a]pyridine- 6-carboxamide 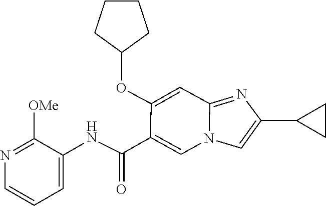 [SMILES: COc1ncccc1NC(=O)c1cn2cc(C3CC3)nc2cc1OC1CCCC1] ArOH-3. RHal: bromocyclopentane | Amount: 3.4 mg, 5.7% LCMS m/z = 391.1; ¹H NMR(DMSO-d₆, 500 MHz) δ: |
| 465 | 7-(Cyclopentyloxy)-2-cyclopropyl-N-(6- methoxypyridin-2-yl)imidazo[1,2-a]pyridine- 6-carboxamide 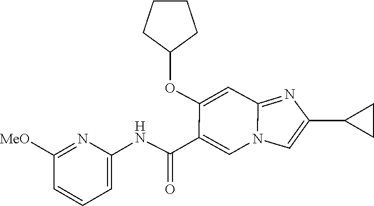 [SMILES: COc1cccc(NC(=O)c2cn3cc(C4CC4)nc3cc2OC2CCCC2)n1] ArOH-4. RHal: bromocyclopentane | Amount: 4.2 mg, 7% LCMS m/z = 393.2; ¹H NMR(DMSO-d₆, 500 MHz) δ: |
| 466 | 2-Cyclopropyl-N-(6-(difluoromethyl)pyridin- 2-yl)-7-isobutoxyimidazo[1,2-a]pyridine-6- carboxamide 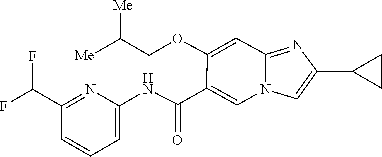 [SMILES: CC(C)COc1cc2nc(C3CC3)cn2cc1C(=O)Nc1cccc(C(F)F)n1] ArOH-5 RHal: 1-bromo-2-methylpropane | Amount: 12.9 mg, 21.5% LCMS m/z = 401.1; ¹H NMR(CDCl₃, 400 MHz) δ: 10.55 (s, 1H), 8.94 (s, 1H), 8.44 (d, 1H), 7.86 (t, 1H), 7.37 (d, 1H), 7.30 (s, 1H), 6.93 (s, 1H), 6.45 (t, 1H), 3.98 (d, 2H), 2.42-2.28 (m, 1H), 2.01-1.97 (m, 1H), 1.19 (d, 6H), 1.06- 0.90 (m, 4H). |
| 467 | 7-(Cyclopentyloxy)-2-cyclopropyl-N-(6- (difluoromethyl)pyridin-2-yl)imidazo[1,2- a]pyridine-6-carboxamide 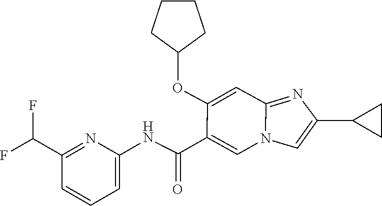 [SMILES: O=C(Nc1cccc(C(F)F)n1)c1cn2cc(C3CC3)nc2cc1OC1CCCC1] ArOH-5. RHal: bromocyclopentane | Amount: 20.4 mg, 34% LCMS m/z = 413.1; ¹H NMR(DMSO-d₆ + CCl₄), 400 MHz) δ: 10.68 (s, 1H), 9.10 (s, 1H), 8.42 (d, 1H), 7.97 (t, 1H), 7.61 (s, 1H), 7.38 (d, 1H), 6.94 (s, 1H), 6.58 (t, 1H), 5.19-5.13 (m, 1H), 2.15-2.04 (m, 4H), 2.02-1.90 (m, 3H), 1.81-1.70 (m, 2H), 0.88 (d, 4H). |
| 468 | 2-Cyclopropyl-N-(6-(difluoromethyl)pyridin- 2-yl)-7-((tetrahydrofuran-3- yl)oxy)imidazo[1,2-a]pyridine-6-carboxamide 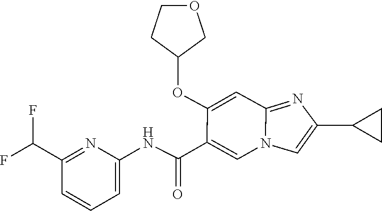 [SMILES: O=C(Nc1cccc(C(F)F)n1)c1cn2cc(C3CC3)nc2cc1OC1CCOC1] ArOH-5. RHal: 3-bromotetrahydrofuran | Amount: 13.5 mg, 22.5% LCMS m/z = 415; ¹H NMR (CDCl₃), 400 MHz) δ: 10.42 (s, 1H), 8.96 (s, 1H), 8.41 (d, 1H), 7.87 (t, 1H), 7.37 (d, 1H), 7.33 (s, 1H), 6.95 (s, 1H), 6.47 (t, 1H), 5.24-5.14 (m, 1H), 4.25-4.17 (m, 2H), 4.11-4.04 (m, 1H), 4.04-3.95 (m, 1H), 2.49-2.41 (m, 2H), 2.03-1.98 (m, 1H), 1.09-0.95 (m, 4H). |
| 469 | 2-Cyclopropyl-N-(6-(difluoromethyl)pyridin- 2-yl)-7-((1-methyl-2-oxopyrrolidin-3- yl)oxy)imidazo[1,2-a]pyridine-6-carboxamide 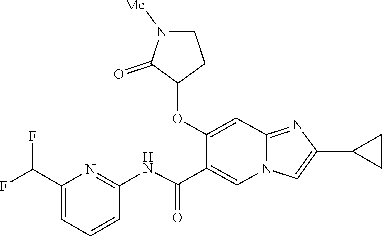 [SMILES: CN1CCC(Oc2cc3nc(C4CC4)cn3cc2C(=O)Nc2cccc(C(F)F)n2)C1=O] ArOH-5 RHal: 3-bromo-1-methylpyrrolidin-2-one | Amount: 8 mg, 13.3% LCMS m/z = 442; ¹H NMR 500 MHz, DMSO-d₆ δ: 10.69 (s, 1H), 8.96 (s, 1H), 8.31 (d, 1H), 8.05 (t, 1H), 7.72 (s, 1H), 7.46 (d, 1H), 7.27 (s, 1H), 6.87 (t, 1H), 5.23 (t, 1H), 3.53-3.46 (m, 1H), 3.42-3.37 (m, 1H), 2.80 (s, 3H), 2.70-2.64 (m, 1H), 2.14-2.08 (m, 1H), 2.02- 1.97 (m, 1H), 0.93-0.88 (m, 2H), 0.85-0.81 (m, 2H). |
| 470 | 7-((7-Oxabicyclo[2.2.1]heptan-2- yl)methoxy)-2-cyclopropyl-N-(6- (difluoromethyl)pyridin-2-yl)imidazo[1,2- a]pyridine-6-carboxamide 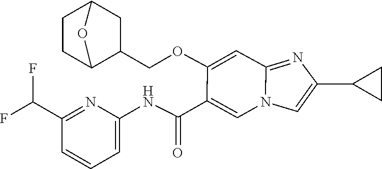 [SMILES: O=C(Nc1cccc(C(F)F)n1)c1cn2cc(C3CC3)nc2cc1OCC1CC2CCC1O2] ArOH-5. RHal: 2-(bromomethyl)-7- oxabicyclo[2.2.1]heptane | Amount: 7.4 mg, 12.3% LCMS m/z = 455.2; ¹H NMR 500 MHz, DMSO-d₆ δ: |
| 471 | 2-Cyclopropyl-7-((3,3- difluorocyclobutyl)methoxy)-N-(6- (difluoromethyl)pyridin-2-yl)imidazo[1,2- a]pyridine-6-carboxamide 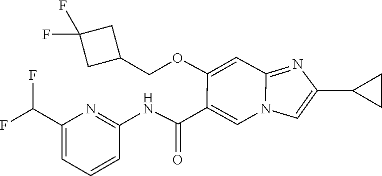 [SMILES: O=C(Nc1cccc(C(F)F)n1)c1cn2cc(C3CC3)nc2cc1OCC1CC(F)(F)C1] ArOH-5. RHal: 3-(bromomethyl)-1,1- difluorocyclobutane | Amount: 5.5 mg, 9.2% LCMS m/z = 449.2; ¹H NMR 500 MHz, DMSO-d₆ δ: |
| 472 | 2-Cyclopropyl-N-(6-(difluoromethyl)pyridin- 2-yl)-7-(3,3- dimethylcyclobutoxy)imidazo[1,2-a]pyridine- 6-carboxamide 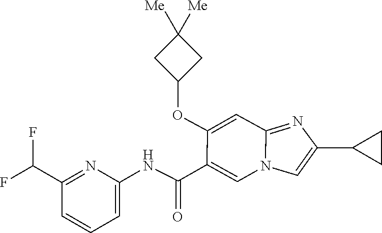 [SMILES: CC1(C)CC(Oc2cc3nc(C4CC4)cn3cc2C(=O)Nc2cccc(C(F)F)n2)C1] ArOH-5. RHal: 3-bromo-1,1- dimethylcyclobutane | Amount: 4.4 mg, 7.3% LCMS m/z = 427.2; ¹H NMR 500 MHz, DMSO-d₆ δ: |
| 473 | 2-Cyclopropyl-N-(6-(difluoromethyl)pyridin- 2-yl)-7-((tetrahydro-2H-pyran-4- yl)oxy)imidazo[1,2-a]pyridine-6-carboxamide 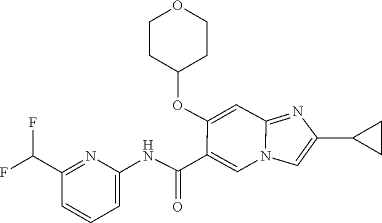 [SMILES: O=C(Nc1cccc(C(F)F)n1)c1cn2cc(C3CC3)nc2cc1OC1CCOCC1] ArOH-5. RHal: 4-(bromomethyl)tetrahydro-2H-pyran | Amount: 5.3 mg, 8.8% LCMS m/z = 429.0; ¹H NMR 500 MHz, DMSO-d₆ δ: |
| 474 | 7-Cyclobutoxy-2-cyclopropyl-N-(6- (difluoromethyl)pyridin-2-yl)imidazo[1,2- a]pyridine-6-carboxamide 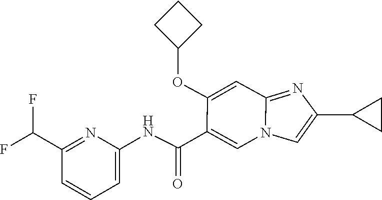 [SMILES: O=C(Nc1cccc(C(F)F)n1)c1cn2cc(C3CC3)nc2cc1OC1CCC1] ArOH-5. RHal: bromocyclobutane | Amount: 7.9 mg, 13.2% LCMS m/z = 399.1; ¹H NMR 600 MHz, DMSO-d₆ δ: 10.76 (s, 1H), 9.00 (s, 1H), 8.34 (d, 1H), 8.04 (t, 1H), 7.69 (s, 1H), 7.44 (d, 1H), 6.87 (t, 1H), 6.76 (s, 1H), 4.94 (p, 1H), 2.58- 2.52 (m, 2H), 2.24-2.14 (m, 2H), 2.00-1.93 (m, 1H), 1.90- 1.81 (m, 1H), 1.75-1.65 (m, 1H), 0.88-0.85 (m, 2H), 0.81- 0.78 (m, 2H). |
| 475 | 2-Cyclopropyl-N-(6-(difluoromethyl)pyridin- 2-yl)-7-((3-methyltetrahydrofuran-3- yl)methoxy)imidazo[1,2-a]pyridine-6- carboxamide 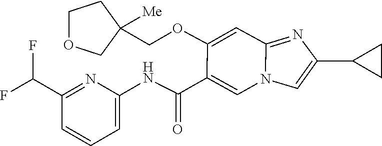 [SMILES: CC1(COc2cc3nc(C4CC4)cn3cc2C(=O)Nc2cccc(C(F)F)n2)CCOC1] ArOH-5 RHal: 3-(bromomethyl)-3- methyltetrahydrofuran | Amount: 3.1 mg, 5.2% LCMS m/z = 443.2; ¹H NMR 600 MHz, DMSO-d₆ δ: |
| 476 | 2-Cyclopropyl-7-(3,3-difluorocyclobutoxy)- N-(6-(difluoromethyl)pyridin-2- yl)imidazo[1,2-a]pyridine-6-carboxamide 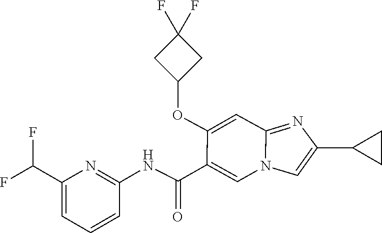 [SMILES: O=C(Nc1cccc(C(F)F)n1)c1cn2cc(C3CC3)nc2cc1OC1CC(F)(F)C1] ArOH-5 RHal: 3-(bromomethyl)-1,1- difluorocyclobutane | Amount: 2.2 mg, 3.7% LCMS m/z = 435.2; ¹H NMR 600 MHz, DMSO-d₆ δ: |
| 477 | 2-Cyclopropyl-7-(3- (difluoromethyl)cyclobutoxy)-N-(6- (difluoromethyl)pyridin-2-yl)imidazo[1,2- a]pyridine-6-carboxamide 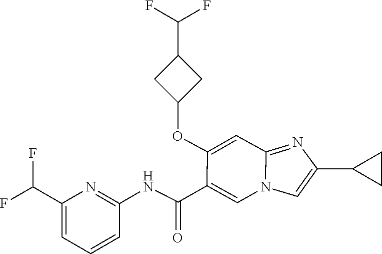 [SMILES: O=C(Nc1cccc(C(F)F)n1)c1cn2cc(C3CC3)nc2cc1OC1CC(C(F)F)C1] ArOH-5. RHal: 1-bromo-3- (difluoromethyl)cyclobutane | Amount: 3.3 mg, 5.5% LCMS m/z = 449.2; ¹H NMR 600 MHz, DMSO-d₆ δ |
| 478 | 7-((7-Oxabicyclo[2.2.1]heptan-2- yl)methoxy)-2-cyclopropyl-N-(6- methoxypyridin-2-yl)imidazo[1,2-a]pyridine- 6-carboxamide 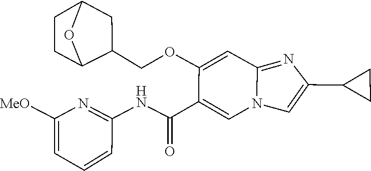 [SMILES: COc1cccc(NC(=O)c2cn3cc(C4CC4)nc3cc2OCC2CC3CCC2O3)n1] ArOH-4. RHal: 2-(bromomethyl)-7- oxabicyclo[2.2.1]heptane | Amount: 4 mg, 6.7% LCMS m/z = 435.2; ¹H NMR 600 MHz, DMSO-d₆ δ: |

### Example 479-543

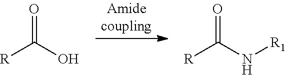

The title compounds were prepared from the appropriate carboxylic acid and amine building block using a one-step library protocol (either Method A or Method B) as described below.

RCO₂H=7-methoxy-2-(tetrahydro-2H-pyran-4-yl)imidazo[1,2-a]pyridine-6-carboxylic acid (Preparation 75), 8-ethoxy-2-(1-methyl-2-oxabicyclo[2.1.1]hexan-4-yl)imidazo[1,2-a]pyrazine-6-carboxylic acid (Preparation 133) or 7-isopropoxy-2-(1-methyl-2-oxabicyclo[2.2.1]heptan-4-yl)imidazo[1,2-a]pyridine-6-carboxylic acid (Preparation 298) or 2-cyclopropyl-7-isopropoxyimidazo[1,2-a]pyridine-6-carboxylic acid (Preparation 299) or 7-cyclobutoxy-2-(1-methyl-2-oxabicyclo[2.1.1]hexan-4-yl)imidazo[1,2-a]pyridine-6-carboxylic acid (Preparation 343) or 7-cyclobutoxy-2-(1-methyl-2-oxabicyclo[2.2.1]heptan-4-yl)imidazo[1,2-a]pyridine-6-carboxylic acid (Preparation 296).

Method A: The appropriate amine building block (1.0 equiv.) was added to a mixture of the appropriate carboxylic acid (1.0 equiv.), EDC (1.05 equiv.), HOAt (1.05 equiv.) and DIPEA (2.5 equiv.) in DMF (1 mL) and stirred at rt for 16 h. The reaction mixture was evaporated to dryness and the residue was dissolved in DMSO (0.5 mL) and purified by prep-HPLC-L, gradient: 0-100% (optimised for each compound) to afford the title compound.

Method B: The appropriate amine building block (1.0 equiv.) and DIPEA (3.14 equiv. +1.0 equiv. per each acid equiv. for amine building block salts) were added to a solution of the appropriate carboxylic acid (1.0 equiv.) in dry DMF (0.5 mL). The resulting mixture was stirred for 30 min at rt followed by the addition of 2-chloro-1-methylpyridin-1-ium iodide (1.32 equiv.) and the mixture stirred for 6 h at 90° C. The reaction mixture was evaporated to dryness in vacuo and the residue dissolved in DMSO (1 mL) and purified by prep-HPLC-L, gradient: 0-100% (optimised for each compound) to afford the title compound.

Method C. MSCl (1.1 equiv.) was added to the mixture of 7-cyclobutoxy-2-(1-methyl-2-oxabicyclo[2.1.1]hexan-4-yl)imidazo[1,2-a]pyridine-6-carboxylic acid (Preparation 343, 1.1 equiv.) in dry DMF (0.5 mL) and the mixture stirred for 1 h at rt. To this was added the appropriate amine building block (1.0 equiv.) and the reaction mixture stirred for 8 h at 50° C. The reaction was evaporated to dryness in vacuo and the residue was dissolved in DMSO (1 mL) and purified by prep-HPLC-G, gradient: 0-100% (optimised for each compound) to afford the title compound.

| Example | Name/Structure/Coupling Method/Data |
|---|---|
| 479 | 7-Methoxy-N-(pyrazolo[1,5-a]pyridin-4-yl)-2-(tetrahydro-2H- pyran-4-yl)imidazo[1,2-a]pyridine-6-carboxamide 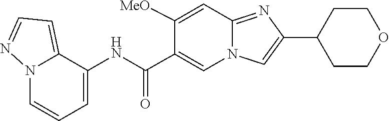 [SMILES: COc1cc2nc(C3CCOCC3)cn2cc1C(=O)Nc1cccn2nccc12] Method A: RNH₂: pyrazolo[1,5-a]pyridin-4-amine LCMS m/z = 392.2 [M + H]⁺; ¹H NMR(DMSO-d₆ + CCl₄, 400 MHz) δ: 1.68-1.82 (m, 2H), 1.91-1.99 (m, 2H), 2.85-2.97 (m, 1H), 3.43-3.53 (m, 2H), 3.91-3.99 (m, 2H), 4.17 (s, 3H), 6.64 (d, 1H), 6.85 (t, 1H), 7.06 (s, 1H), 7.59 (s, 1H), 7.89 (d, 1H), 8.10 (d, 1H), 8.34 (d, 1H), 9.10 (s, 1H), 10.11 (s, 1H). |
| 480 | 2-Cyclopropyl-N-(1-ethyl-2-oxo-1,2-dihydropyridin-3-yl)-7- isopropoxyimidazo[1,2-a]pyridine-6-carboxamide 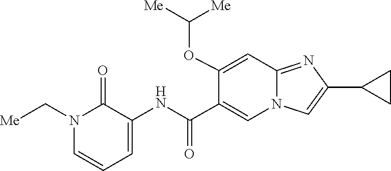 [SMILES: CCn1cccc(NC(=O)c2cn3cc(C4CC4)nc3cc2OC(C)C)c1=O] Method A: RNH₂: 3-amino-1-ethylpyridin-2(1H)-one Amount: 7.5 mg, 15% Gradient: 40-90% LCMS m/z = 381.2 [M + H]⁺. |
| 481 | 2-Cyclopropyl-N-(6-ethoxypyridin-2-yl)-7-isopropoxyimidazo[1,2- a]pyridine-6-carboxamide 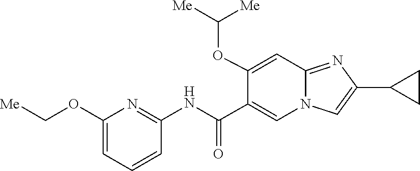 [SMILES: CCOc1cccc(NC(=O)c2cn3cc(C4CC4)nc3cc2OC(C)C)n1] Method A: RNH₂: 6-ethoxypyridin-2-amine Amount: 3.1 mg, 6.2%. Gradient: 60-100% LCMS m/z = 381.2 [M + H]⁺. |
| 482 | 2-Cyclopropyl-N-(6-(2,2-difluoroethoxy)pyridin-2-yl)-7- isopropoxyimidazo[1,2-a]pyridine-6-carboxamide 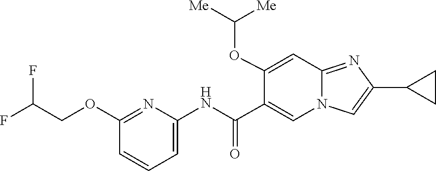 [SMILES: CC(C)Oc1cc2nc(C3CC3)cn2cc1C(=O)Nc1cccc(OCC(F)F)n1] Method A: RNH₂: 6-(2,2-difluoroethoxy)pyridin-2-amine Amount: 1.5 mg, 3%. Gradient: 60-100% LCMS m/z = 417.2 [M + H]⁺. |
| 483 | 2-Cyclopropyl-N-(6-(hydroxymethyl)pyridin-2-yl)-7- isopropoxyimidazo[1,2-a]pyridine-6-carboxamide 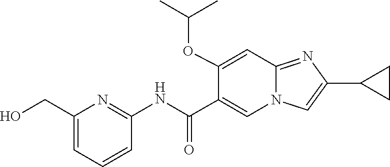 [SMILES: CC(C)Oc1cc2nc(C3CC3)cn2cc1C(=O)Nc1cccc(CO)n1] Method A: RNH₂: 6-hydroxymethyl-2-aminopyridine Amount: 1.7 mg, 3.4%. Gradient: 40-90% LCMS m/z = 369.0 [M + H]⁺. |
| 484 | 8-Ethoxy-N-(5-fluoro-2-methoxyphenyl)-2-(1-methyl-2- oxabicyclo[2.1.1]hexan-4-yl)imidazo[1,2-a]pyrazine-6-carboxamide 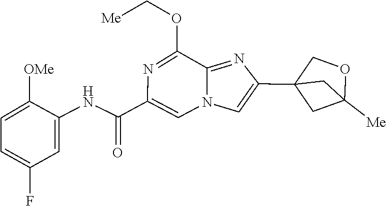 [SMILES: CCOc1nc(C(=O)Nc2cc(F)ccc2OC)cn2cc(C34COC(C)(C3)C4)nc12] Method A: RNH₂: 5-fluoro-2-methoxy aniline Yield: 5.2 mg LCMS m/z = 427.2 [M + H]⁺. |
| 485 | N-(2-chloro-3-fluorophenyl)-8-ethoxy-2-(1-methyl-2- oxabicyclo[2.1.1]hexan-4-yl)imidazo[1,2-a]pyrazine-6-carboxamide 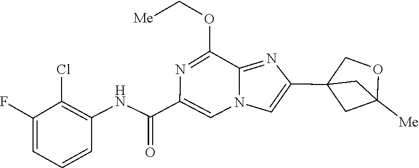 [SMILES: CCOc1nc(C(=O)Nc2cccc(F)c2Cl)cn2cc(C34COC(C)(C3)C4)nc12] Method B: RNH₂: 2-chloro-3-fluoroaniline Yield: 6.6 mg, 11% LCMS m/z = 431.2 [M + H]⁺. |
| 486 | 8-Ethoxy-N-(6-methoxypyridin-2-yl)-2-(1-methyl-2- oxabicyclo[2.1.1]hexan-4-yl)imidazo[1,2-a]pyrazine-6-carboxamide 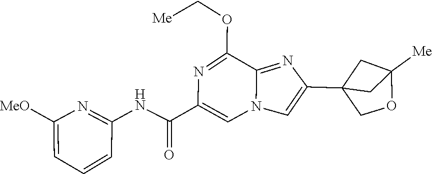 [SMILES: CCOc1nc(C(=O)Nc2cccc(OC)n2)cn2cc(C34COC(C)(C3)C4)nc12] METHOD B: RNH₂: 6-methoxy-2-aminopyridine Yield: 4.8 mg, 8% LCMS m/z = 410.2 [M + H]⁺. |
| 487 | 8-Ethoxy-N-(3-fluoro-2-methoxyphenyl)-2-(1-methyl-2- oxabicyclo[2.1.1]hexan-4-yl)imidazo[1,2-a]pyrazine-6-carboxamide 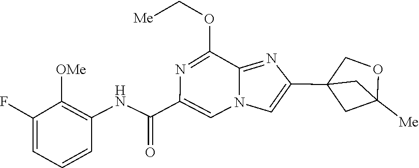 [SMILES: CCOc1nc(C(=O)Nc2cccc(F)c2OC)cn2cc(C34COC(C)(C3)C4)nc12] Method B: RNH₂: 2-methoxy-3-fluoroaniline. Yield: 18.8 mg, 31.3% LCMS m/z = 427.2 [M + H]⁺; ¹H NMR(DMSO-d₆ + CCl₄, 400 MHz) δ: 10.24 (s, 1H), 8.96 (s, 1H), 8.27 (d, 1H), 7.98 (s, 1H), 7.08-7.02 (m, 1H), 6.87 (t, 1H), 4.72-4.66 (m, 2H), 4.03 (s, 3H), 3.91 (s, 2H), 2.05- 2.01 (m, 2H), 1.83-1.79 (m, 2H), 1.64-1.57 (m, 3H), 1.45 (s, 3H). |
| 488 | N-(3-chloro-2-fluorophenyl)-8-ethoxy-2-(1-methyl-2- oxabicyclo[2.1.1]hexan-4-yl)imidazo[1,2-a]pyrazine-6-carboxamide 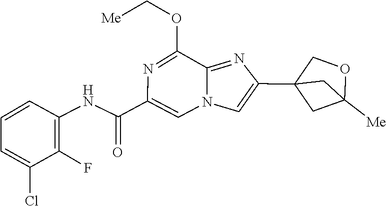 [SMILES: CCOc1nc(C(=O)Nc2cccc(Cl)c2F)cn2cc(C34COC(C)(C3)C4)nc12] Method B: RNH₂: 2-fluoro-3-chloroaniline. Yield: 18.8 mg, 31.5% LCMS m/z = 431.0 [M + H]⁺; ¹H NMR(DMSO-d₆ + CCl₄, 400 MHz) δ: 9.93 (s, 1H), 8.97 (s, 1H), 8.33-8.25 (m, 1H), 7.97 (s, 1H), 7.24-7.15 (m, 2H), 4.66 (q, 2H), 3.91 (s, 2H), 2.03 (d, 2H), 1.81 (d, 2H), 1.58 (t, 3H), 1.44 (s, 3H). |
| 489 | 8-Ethoxy-2-(1-methyl-2-oxabicyclo[2.1.1]hexan-4-yl)-N- (pyrazolo[1,5-a]pyridin-7-yl)imidazo[1,2-a]pyrazine-6- carboxamide 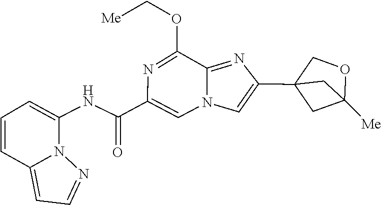 [SMILES: CCOc1nc(C(=O)Nc2cccc3ccnn23)cn2cc(C34COC(C)(C3)C4)nc12] Method B: RNH₂: pyrazolo[1,5-a]pyridin-7-amine. Yield: 8.8 mg, 14.5% LCMS m/z = 419.1 [M + H]⁺. |
| 490 | N-(1-(difluoromethyl)-1H-pyrazol-3-yl)-8-ethoxy-2-(1-methyl-2- oxabicyclo[2.1.1]hexan-4-yl)imidazo[1,2-a]pyrazine-6-carboxamide 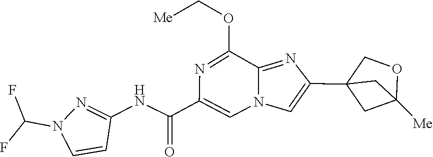 [SMILES: CCOc1nc(C(=O)Nc2ccn(C(F)F)n2)cn2cc(C34COC(C)(C3)C4)nc12] Method B: RNH₂: 1-(difluoromethyl)-1H-pyrazol-3-amine. Yield: 17.2 mg, 28.7%. LCMS m/z = 419.1 [M + H]⁺; ¹H NMR(CDCl₃, 400 MHz) δ: 9.80 (s, 1H), 8.66 (s, 1H), 7.79 (s, 1H), 7.59 (s, 1H), 7.26-6.93 (m, 2H), 4.74 (q, 2H), 4.10 (s, 2H), 2.13-2.09 (m, 2H), 2.03-1.98 (m, 2H), 1.61 (t, 3H), 1.53 (s, 3H). |
| 491 | 8-Ethoxy-N-(4-fluoro-2-methoxyphenyl)-2-(1-methyl-2- oxabicyclo[2.1.1]hexan-4-yl)imidazo[1,2-a]pyrazine-6-carboxamide 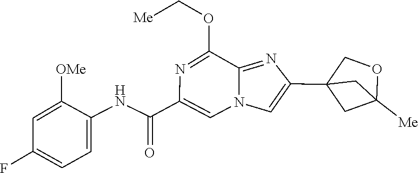 [SMILES: CCOc1nc(C(=O)Nc2ccc(F)cc2OC)cn2cc(C34COC(C)(C3)C4)nc12] Method A: RNH₂: 4-fluoro-2-methoxyaniline. Yield: 3.3 mg. 5.5% LCMS m/z = 427.2 [M + H]⁺. |
| 492 | N-(2-(difluoromethoxy)pyridin-3-yl)-8-ethoxy-2-(1-methyl-2- oxabicyclo[2.1.1]hexan-4-yl)imidazo[1,2-a]pyrazine-6-carboxamide 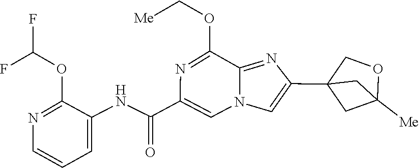 [SMILES: CCOc1nc(C(=O)Nc2cccnc2OC(F)F)cn2cc(C34COC(C)(C3)C4)nc12] Method B: RNH₂: 2-(difluoromethoxy)pyridin-3-amine Yield: 2.1 mg, 3.4%. LCMS m/z = 446.2 [M + H]⁺. |
| 493 | N-(6-cyclopropylpyridin-2-yl)-8-ethoxy-2-(1-methyl-2- oxabicyclo[2.1.1]hexan-4-yl)imidazo[1,2-a]pyrazine-6-carboxamide 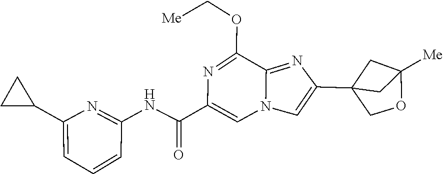 [SMILES: CCOc1nc(C(=O)Nc2cccc(C3CC3)n2)cn2cc(C34COC(C)(C3)C4)nc12] Method B: RNH₂: 6-cyclopropyl-2-aminopyridine Yield: 3.4 mg, 5.3%. LCMS m/z = 420.2 [M + H]⁺. |
| 494 | N-(3-(difluoromethyl)phenyl)-8-ethoxy-2-(1-methyl-2- oxabicyclo[2.1.1]hexan-4-yl)imidazo[1,2-a]pyrazine-6-carboxamide 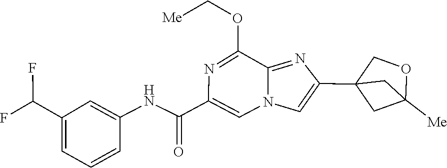 [SMILES: CCOc1nc(C(=O)Nc2cccc(C(F)F)c2)cn2cc(C34COC(C)(C3)C4)nc12] Method B RNH₂: 3-(difluoromethyl)aniline Yield: 20.7 mg, 34.5% LCMS m/z = 429.1 [M + H]⁺; ¹H NMR(CDCl₃, 400 MHz) δ: 9.52 (s, 1H), 8.67 (s, 1H), 7.89 (s, 1H), 7.84 (d, 1H), 7.60 (s, 1H), 7.49 (t, 1H), 7.32 (d, 1H), 6.68 (t, 1H), 4.76 (q, 2H), 4.10 (s, 2H), 2.14-2.09 (m, 2H), 2.03-1.96 (m, 2H), 1.64 (t, 3H), 1.53 (s, 3H). |
| 495 | N-(3-chlorophenyl)-8-ethoxy-2-(1-methyl-2-oxabicyclo[2.1.1]hexan-4- |
| | yl)imidazo[1,2-a]pyrazine-6-carboxamide 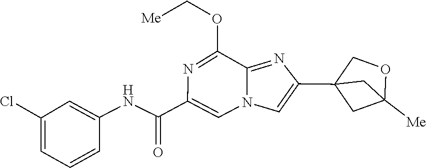 [SMILES: CCOc1nc(C(=O)Nc2cccc(Cl)c2)cn2cc(C34COC(C)(C3)C4)nc12] Method B: RNH₂: 3-chloroaniline. Yield: 20.4 mg, 34% LCMS m/z = 413.2 [M + H]⁺; ¹H NMR (CDCl₃, 400 MHz) δ: 9.44 (s, 1H), 8.67 (s, 1H), 7.82 (s, 1H), 7.62-7.54 (m, 2H), 7.32 (t, 1H), 7.14 (d, 1H), 4.75 (q, 2H), 4.10 (s, 2H), 2.15-2.09 (m, 2H), 2.02-1.96 (m, 2H), 1.63 (t, 3H), 1.53 (s, 3H). |
| 496 | 8-Ethoxy-N-(2-fluoro-3-methylphenyl)-2-(1-methyl-2- oxabicyclo[2.1.1]hexan-4-yl)imidazo[1,2-a]pyrazine-6-carboxamide 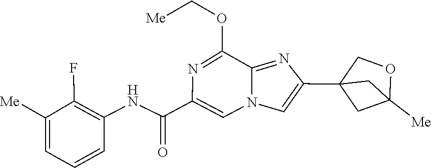 [SMILES: CCOc1nc(C(=O)Nc2cccc(C)c2F)cn2cc(C34COC(C)(C3)C4)nc12] Method B: RNH₂: 2-fluoro3-methylaniline. Yield: 2.6 mg, 4.4% LCMS m/z = 411.2 [M + H]⁺. |
| 497 | 8-Ethoxy-2-(1-methyl-2-oxabicyclo[2.1.1]hexan-4-yl)-N-(3- (trifluoromethyl)phenyl)imidazo[1,2-a]pyrazine-6-carboxamide 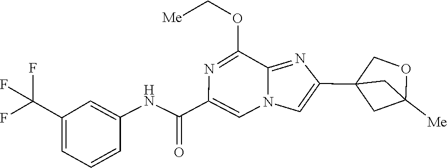 [SMILES: CCOc1nc(C(=O)Nc2cccc(C(F)(F)F)c2)cn2cc(C34COC(C)(C3)C4)nc12] Method B: RNH₂: 3-trifluoromethylaniline. Yield: 12.4 mg, 20.7% LCMS m/z = 447.2 [M + H]⁺; ¹H NMR(DMSO-d₆ + CCl₄, 400 MHz) δ: 1.46 (s, 3H), 1.57 (t, 3H), 1.80-1.86 (m, 2H), 2.02-2.07 (m, 2H), 3.93 (s, 2H), 4.79 (q, 2H), 7.38 (d, 1H), 7.54 (t, 1H), 7.99 (s, 1H), 8.12 (d, 1H), 8.25 (s, 1H), 8.94 (s, 1H), 10.09 (s, 1H). |
| 498 | 8-Ethoxy-2-(1-methyl-2-oxabicyclo[2.1.1]hexan-4-yl)-N-(6- methylpyrazolo[1,5-a]pyrimidin-3-yl)imidazo[1,2-a]pyrazine-6- carboxamide 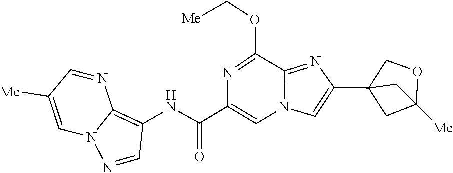 [SMILES: CCOc1nc(C(=O)Nc2cnn3cc(C)cnc23)cn2cc(C34COC(C)(C3)C4)nc12] Method B: RNH₂: 6-methylpyrazolo[1,5-a]pyrimidin-3-amine Yield: 3.3 mg, 5.5% LCMS m/z = 434.2 [M + H]⁺. |
| 499 | 8-Ethoxy-N-(2-isopropoxypyridin-3-yl)-2-(1-methyl-2- oxabicyclo[2.1.1]hexan-4-yl)imidazo[1,2-a]pyrazine-6-carboxamide 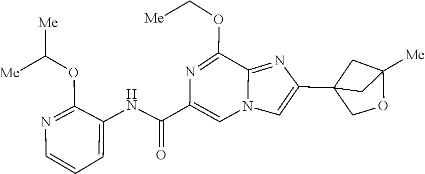 [SMILES: CCOc1nc(C(=O)Nc2cccnc2OC(C)C)cn2cc(C34COC(C)(C3)C4)nc12] Method B: RNH₂: 2-isopropoxypyridin-3-amine. Yield: 3.9 mg, 6.4% LCMS m/z = 438.2 [M + H]⁺. |
| 500 | 8-Ethoxy-N-(1-methyl-2-oxo-1,2-dihydropyridin-3-yl)-2-(1-methyl-2- oxabicyclo[2.1.1]hexan-4-yl)imidazo[1,2-a]pyrazine-6-carboxamide 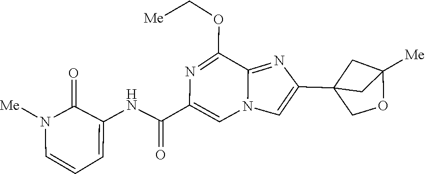 [SMILES: CCOc1nc(C(=O)Nc2cccn(C)c2=O)cn2cc(C34COC(C)(C3)C4)nc12] Method B: RNH₂: 3-amino-1-methylpyridin-2(1H)-one Yield: 6.5 mg, 10.8% LCMS m/z = 410.2 [M + H]⁺. |
| 501 | 8-Ethoxy-N-(1-ethyl-1H-pyrazol-3-yl)-2-(1-methyl-2- oxabicyclo[2.1.1]hexan-4-yl)imidazo[1,2-a]pyrazine-6-carboxamide 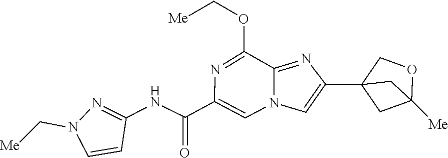 [SMILES: CCOc1nc(C(=O)Nc2ccn(CC)n2)cn2cc(C34COC(C)(C3)C4)nc12] Method B: RNH₂: 1-ethyl-1H-pyrazol-3-amine. Yield: 9.1 mg, 15.1% LCMS m/z = 397.2 [M + H]⁺. |
| 502 | N-(2-fluoro-3-(trifluoromethyl)phenyl)-7-isopropoxy-2-(1-methyl-2- oxabicyclo[2.2.1]heptan-4-yl)imidazo[1,2-a]pyridine-6-carboxamide 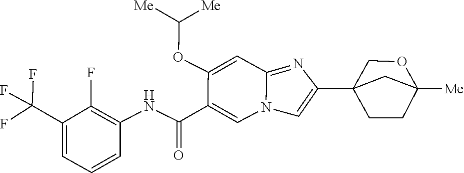 [SMILES: CC(C)Oc1cc2nc(C34CCC(C)(C3)OC4)cn2cc1C(=O)Nc1cccc(C(F)(F)F)c1F] Method B: RNH₂: 2-fluoro-3-(trifluoromethyl)aniline. Yield: 15.3 mg, 15.3% LCMS m/z = 492.2 [M + H]⁺, ¹H NMR(400 MHz, DMSO-d₆ + CCl₄) δ: 10.38 (s, 1H), 9.20 (s, 1H), 8.87-8.70 (m, 1H), 7.66 (s, 1H), 7.39-7.27 (m, 2H), 7.11 (s, 1H), 5.11-4.88 (m, 1H), 4.00-3.88 (m, 1H), 3.73 (d, 1H), 2.12-2.02 (m, 1H), 1.99-1.88 (m, 1H), 1.86-1.68 (m, 4H), 1.52 (d, 6H), 1.38 (s, 3H). |
| 503 | 7-Isopropoxy-2-(1-methyl-2-oxabicyclo[2.2.1]heptan-4-yl)-N-(4- (trifluoromethyl)thiazol-2-yl)imidazo[1,2-a]pyridine-6-carboxamide 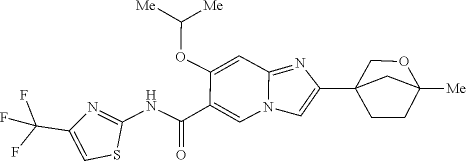 [SMILES: CC(C)Oc1cc2nc(C34CCC(C)(C3)OC4)cn2cc1C(=O)Nc1nc(C(F)(F)F)cs1] Method B: RNH₂: 4-(trifluoromethyl)thiazol-2-amineYield: 6.7 mg, 6.7% LCMS m/z = 481.2 [M + H]⁺, ¹H NMR(600 MHz, DMSO-d₆) δ: 12.31 (s, 1H), 8.94 (s, 1H), 8.03 (s, 1H), 7.68 (s, 1H), 7.08 (s, 1H), 4.87-4.74 (m, 1H), 3.94-3.87 (m, 1H), 3.73 (d, 1H), 2.04-2.00 (m, 1H), 1.93-1.88 (m, 1H), 1.81-1.73 (m, 3H), 1.68-1.63 (m, 1H), 1.37-1.33 (m, 9H) |
| 504 | N-(1-(difluoromethyl)-1H-pyrazol-3-yl)-7-isopropoxy-2-(1-methyl-2- oxabicyclo[2.2.1]heptan-4-yl)imidazo[1,2-a]pyridine-6-carboxamide 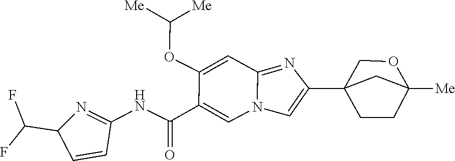 [SMILES: CC(C)Oc1cc2nc(C34CCC(C)(C3)OC4)cn2cc1C(=O)NC1=NC(C(F)F)C=C1] Method B: RNH₂: 1-(difluoromethyl)-1H-pyrazol-3-amine Yield: 14 mg, 14.% LCMS m/z = 446.2 [M + H]⁺, ¹H NMR(400 MHz, DMSO-d₆ + CCl₄) δ: 10.45 (s, 1H), 9.08 (s, 1H), 7.98 (d, 1H), 7.69-7.29 (m, 2H), 7.02 (s, 1H), 6.93 (d, 1H), 5.00-4.85 (m, 1H), 3.97-3.86 (m, 1H), 3.71 (d, 1H), 2.11-2.02 (m, 1H), 1.97-1.87 (m, 1H), 1.84-1.68 (m, 4H), 1.52 (d, 6H), 1.37 (s, 3H). |
| 505 | 7-Isopropoxy-N-(1-methyl-2-oxo-1,2-dihydropyridin-3-yl)-2-(1-methyl-2- oxabicyclo[2.2.1]heptan-4-yl)imidazo[1,2-a]pyridine-6-carboxamide 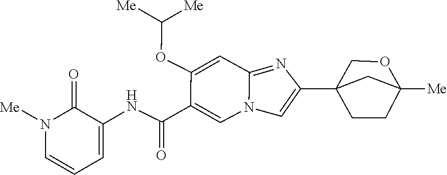 [SMILES: CC(C)Oc1cc2nc(C34CCC(C)(C3)OC4)cn2cc1C(=O)Nc1cccn(C)c1=O] Method B: RNH₂: 3-amino-1-methylpyridin-2(1H)-one. Yield: 20.4 mg, 20.4%. LCMS m/z = 437.2 [M + H]⁺, ¹HNMR(400 MHz, DMSO-d₆ + CCl₄) δ: 10.71 (s, 1H), 9.10 (s, 1H), 8.45 (dd, 1H), 7.58 (s, 1H), 7.29 (dd, 1H), 6.99 (s, 1H), 6.24-6.13 (m, 1H), 5.01-4.87 (m, 1H), 3.96-3.86 (m, 1H), 3.77-3.67 (m, 1H), 3.56 (s, 3H), 2.12-2.01 (m, 1H), 1.98-1.87 (m, 1H), 1.86-1.67 (m, 4H), 1.57 (d, 6H), 1.37 (s, 3H). |
| 506 | 7-Isopropoxy-2-(1-methyl-2-oxabicyclo[2.2.1]heptan-4-yl)-N-(1- (trifluoromethyl)-1H-pyrazol-3-yl)imidazo[1,2-a]pyridine-6-carboxamide 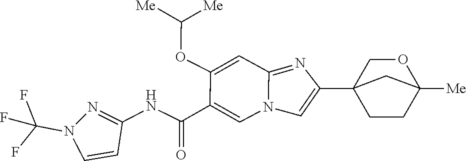 [SMILES: CC(C)Oc1cc2nc(C34CCC(C)(C3)OC4)cn2cc1C(=O)Nc1ccn(C(F)(F)F)n1] Method A: RNH₂: 1-(trifluoromethyl)-1H-pyrazol-3-amine. Yield: 6.3 mg, 6.3% LCMS m/z = 464.2 [M + H]⁺. |
| 507 | 7-Isopropoxy-2-(1-methyl-2-oxabicyclo[2.2.1]heptan-4-yl)-N-(3- methylisoxazol-4-yl)imidazo[1,2-a]pyridine-6-carboxamide 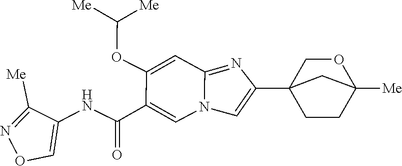 [SMILES: Cc1nocc1NC(=O)c1cn2cc(C34CCC(C)(C3)OC4)nc2cc1OC(C)C] Method B: RNH₂: 3-methylisoxazol-4-amine. Yield: 15.4 mg, 15.4% LCMS m/z = 411.0 [M + H]⁺, ¹H NMR(400 MHz, CDCl₃) δ: 9.54 (s, 1H), 9.14 (s, 1H), 9.02 (s, 1H), 7.35 (s, 1H), 7.01 (s, 1H), 4.93- 4.78 (m, 1H), 4.16-4.04 (m, 1H), 3.96 (d, 1H), 2.41 (s, 3H), 2.20-2.06 (m, 2H), 2.02-1.90 (m, 2H), 1.89-1.78 (m, 2H), 1.58 (d, 6H), 1.48 (s, 3H). |
| 508 | N-(1-ethyl-1H-pyrazol-3-yl)-7-isopropoxy-2-(1-methyl-2- oxabicyclo[2.2.1]heptan-4-yl)imidazo[1,2-a]pyridine-6-carboxamide 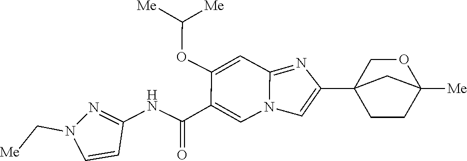 [SMILES: CCn1ccc(NC(=O)c2cn3cc(C45CCC(C)(C4)OC5)nc3cc2OC(C)C)n1] Method A: RNH₂: 1-ethyl-1H-pyrazol-3-amine. Yield: 23 mg, 23 mg LCMS m/z = 424.2 [M + H]⁺, ¹H NMR(400 MHz, DMSO-d₆ + CCl₄) δ: 10.20 (s, 1H), 9.08 (s, 1H), 7.59 (s, 1H), 7.45 (d, 1H), 7.01 (s, 1H), 6.59 (d, 1H), 5.02-4.86 (m, 1H), 4.13-3.98 (m, 2H), 3.97-3.87 (m, 1H), 3.71 (d, 1H), 2.11-2.01 (m, 1H), 2.00-1.87 (m, 1H), 1.84-1.68 (m, 4H), 1.54 (d, 6H), 1.42 (t, 3H), 1.37 (s, 3H). |
| 509 | N-(6-cyanopyridin-2-yl)-7-isopropoxy-2-(1-methyl-2- oxabicyclo[2.2.1]heptan-4-yl)imidazo[1,2-a]pyridine-6-carboxamide 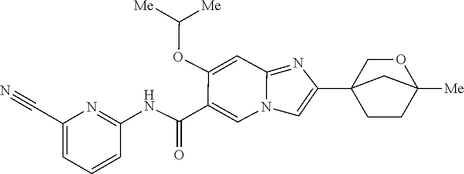 [SMILES: CC(C)Oc1cc2nc(C34CCC(C)(C3)OC4)cn2cc1C(=O)Nc1cccc(C#N)n1] Method B: RNH₂: 6-cycano-2-aminopyridine. Yield: 8.7 mg, 8.7% LCMS m/z = 432.2 [M + H]⁺ |
| 510 | 7-Isopropoxy-2-(1-methyl-2-oxabicyclo[2.2.1]heptan-4-yl)-N-(3- (trifluoromethyl)phenyl)imidazo[1,2-a]pyridine-6-carboxamide 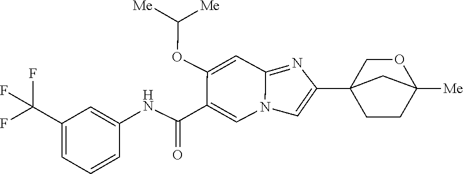 [SMILES: CC(C)Oc1cc2nc(C34CCC(C)(C3)OC4)cn2cc1C(=O)Nc1cccc(C(F)(F)F)c1] Method B: RNH₂: 3-trifluoromethylaniline. Yield: 24.3 mg, 24.3% LCMS m/z = 474.2 [M + H]⁺, ¹H NMR(400 MHz, DMSO-d₆ + CCl₄) δ: 10.23 (s, 1H), 8.97 (s, 1H), 8.19 (s, 1H), 7.79 (d, 1H), 7.61 (s, 1H), 7.52 (t, 1H), 7.36 (d, 1H), 7.00 (s, 1H), 4.95-4.83 (m, 1H), 3.99-3.89 (m, 1H), 3.74 (d, 1H), 2.16-2.03 (m, 1H), 2.00-1.91 (m, 1H), 1.87-1.69 (m, 4H), 1.52 (d, 6H), 1.40 (s, 3H). |
| 511 | N-(4-ethylthiazol-5-yl)-7-isopropoxy-2-(1-methyl-2- oxabicyclo[2.2.1]heptan-4-yl)imidazo[1,2-a]pyridine-6-carboxamide 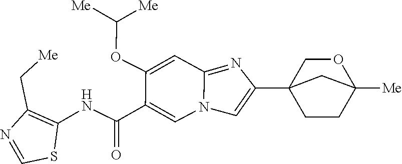 [SMILES: CCc1ncsc1NC(=O)c1cn2cc(C34CCC(C)(C3)OC4)nc2cc1OC(C)C] Method B: RNH₂: 4-ethylthiazol-5-amine. Yield: 13.5 mg, 13.5% LCMS m/z = 441.2 [M + H]⁺, ¹HNMR (400 MHz, CDCl₄) δ: 10.27 (s, 1H), 9.07 (s, 1H), 8.46 (s, 1H), 7.35 (s, 1H), 7.03 (s, 1H), 4.98- 4.82 (m, 1H), 4.17-4.06 (m, 1H), 3.97 (d, 1H), 2.85 (q, 2H), 2.22-2.05 (m, 2H), 2.02-1.92 (m, 2H), 1.89-1.78 (m, 2H), 1.60 (d, 6H), 1.49 (s, 3H), 1.38 (t, 3H). |
| 512 | 7-Isopropoxy-2-(1-methyl-2-oxabicyclo[2.2.1]heptan-4-yl)-N- (thiazol-2-yl)imidazo[1,2-a]pyridine-6-carboxamide 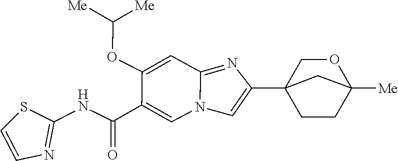 [SMILES: CC(C)Oc1cc2nc(C34CCC(C)(C3)OC4)cn2cc1C(=O)Nc1nccs1] Method B: RNH₂: 2-aninothiazole. Yield: 13.5 mg, 13.5% LCMS m/z = 413.1 [M + H]⁺, ¹HNMR(400 MHz, CDCl₃) δ: 11.24 (s, 1H), 9.05 (s, 1H), 7.53 (d, 1H), 7.35 (s, 1H), 7.04 (d, 1H), 6.99 (s, 1H), 4.89-4.76 (m, 1H), 4.14-4.06 (m, 1H), 3.96 (d, 1H), 2.20- 2.05 (m, 2H), 2.02-1.90 (m, 2H), 1.88-1.81 (m, 2H), 1.61 (d, 6H), 1.49 (s, 3H). |
| 513 | 7-Isopropoxy-N-(3-methoxyisothiazol-4-yl)-2-(1-methyl-2- oxabicyclo[2.2.1]heptan-4-yl)imidazo[1,2-a]pyridine-6-carboxamide 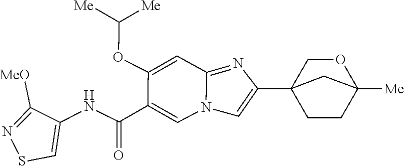 [SMILES: COc1nscc1NC(=O)c1cn2cc(C34CCC(C)(C3)OC4)nc2cc1OC(C)C] Method B: RNH₂: 3-methoxyisoxazol-4-amine. Yield: 18.8 mg, 18.8% LCMS m/z = 443.2 [M + H]⁺, ¹H NMR(400 MHz, DMSO-d₆ + CCl₄) δ: 10.28 (s, 1H), 9.20 (s, 1H), 8.95 (s, 1H), 7.64 (s, 1H), 7.08 (s, 1H), 5.06-4.95 (m, 1H), 4.11 (s, 3H), 3.99-3.90 (m, 1H), 3.73 (d, 1H), 2.14- 2.01 (m, 1H), 2.01-1.88 (m, 1H), 1.88-1.67 (m, 4H), 1.54 (d, 6H), 1.39 (s, 3H). |
| 514 | N-(3-(difluoromethyl)phenyl)-7-isopropoxy-2-(1-methyl-2- oxabicyclo[2.2.1]heptan-4-yl)imidazo[1,2-a]pyridine-6-carboxamide 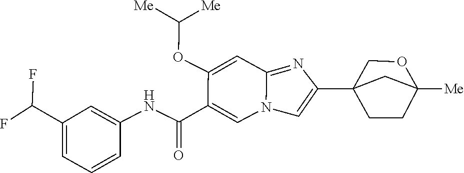 [SMILES: CC(C)Oc1cc2nc(C34CCC(C)(C3)OC4)cn2cc1C(=O)Nc1cccc(C(F)F)c1] Method B: RNH₂: 3-(difluoromethyl)aniline. Yield: 16 mg, 16% LCMS m/z = 456.2 [M + H]⁺, ¹H NMR(400 MHz, DMSO-d₆ + CCl₄) δ: 10.16 (s, 1H), 9.00 (s, 1H), 7.99 (s, 1H), 7.72 (d, 1H), 7.61 (s, 1H), 7.44 (t, 1H), 7.27 (d, 1H), 7.10-6.62 (m, 2H), 4.99-4.81 (m, 1H), 3.98-3.90 (m, 1H), 3.74 (d, 1H), 2.16-2.02 (m, 1H), 1.95 (s, 1H), 1.86-1.69 (m, 4H), 1.53 (d, 6H), 1.40 (s, 3H). |
| 515 | N-(2,3-difluorophenyl)-7-isopropoxy-2-(1-methyl-2- oxabicyclo[2.2.1]heptan-4-yl)imidazo[1,2-a]pyridine-6-carboxamide 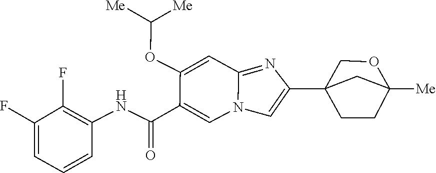 [SMILES: CC(C)Oc1cc2nc(C34CCC(C)(C3)OC4)cn2cc1C(=O)Nc1cccc(F)c1F] Method B: RNH₂: 2,3-difluoroaniline. Yield: 19 mg, 19% LCMS m/z = 442.2 [M + H]⁺, ¹H NMR (400 MHz, DMSO-d₆ + CCl₄) δ: 10.33 (s, 1H), 9.20 (s, 1H), 8.32 (t, 1H), 7.66 (s, 1H), 7.22-7.12 (m, 1H), 7.11 (s, 1H), 7.07-6.93 (m, 1H), 5.09-4.95 (m, 1H), 4.00-3.89 (m, 1H), 3.74 (d, 1H), 2.13-2.03 (m, 1H), 2.01-1.91 (m, 1H), 1.88-1.66 (m, 4H), 1.54 (d, 6H), 1.40 (s, 3H). |
| 516 | N-(4-(difluoromethyl)thiazol-2-yl)-7-isopropoxy-2-(1-methyl-2- oxabicyclo[2.2.1]heptan-4-yl)imidazo[1,2-a]pyridine-6-carboxamide 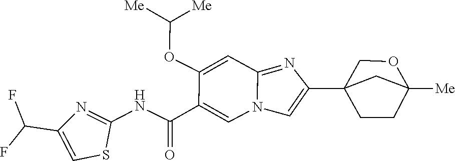 [SMILES: CC(C)Oc1cc2nc(C34CCC(C)(C3)OC4)cn2cc1C(=O)Nc1nc(C(F)F)cs1] Method B: RNH₂: 4-(difluoromethyl)thiazol-2-amine Yield: 6.9 mg, 6.9% LC. MS m/z = 463.2 [M + H]⁺, ¹H NMR(400 MHz, DMSO-d₆) δ: 12.09 (s, 1H), 8.98 (s, 1H), 7.74-7.65 (m, 2H), 7.21-6.83 (m, 2H), 4.92-4.76 (m, 1H), 3.98-3.89 (m, 1H), 3.75 (d, 1H), 2.12-1.99 (m, 1H), 1.99-1.86 (m, 1H), 1.86-1.61 (m, 4H), 1.39 (s, 3H), 1.37 (s, 6H). |
| 517 | N-(5-fluoropyridin-2-yl)-7-isopropoxy-2-(1-methyl-2- oxabicyclo[2.2.1]heptan-4-yl)imidazo[1,2-a]pyridine-6-carboxamide 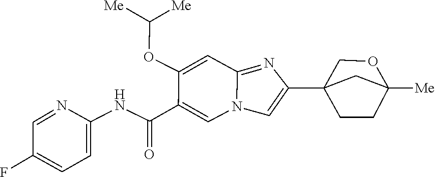 [SMILES: CC(C)Oc1cc2nc(C34CCC(C)(C3)OC4)cn2cc1C(=O)Nc1ccc(F)cn1] Method B: RNH₂: 5-fluoro-2-aminopyridine. Yield: 3.4 mg, 3.4% LCMS m/z = 425.2 [M + H]⁺ |
| 518 | 7-Isopropoxy-2-(1-methyl-2-oxabicyclo[2.2.1]heptan-4-yl)-N- (pyridin-2-yl)imidazo[1,2-a]pyridine-6-carboxamide 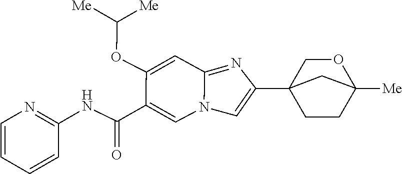 [SMILES: CC(C)Oc1cc2nc(C34CCC(C)(C3)OC4)cn2cc1C(=O)Nc1ccccn1] Method B: RNH₂: 2-aminopyridine. Yield: 2.8 mg, 2.8% LCMS m/z = 407.2 [M + H]⁺ |
| 519 | N-(6-(cyanomethyl)pyridin-2-yl)-2-cyclopropyl-7-isopropoxy- imidazo[1,2-a]pyridine-6-carboxamide 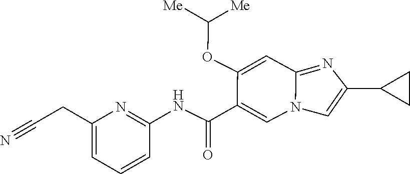 [SMILES: CC(C)Oc1cc2nc(C3CC3)cn2cc1C(=O)Nc1cccc(CC#N)n1] Method B: RNH₂: 6-cycanomethyl-2-aminopyridine LCMS m/z = 376.0 [M + H]⁺. |
| 520 | 7-Cyclobutoxy-N-(6-(1-methyl-1H-pyrazol-4-yl)pyridin-2-yl)-2-(1-methyl-2- oxabicyclo[2.1.1]hexan-4-yl)imidazo[1,2-a]pyridine-6-carboxamide 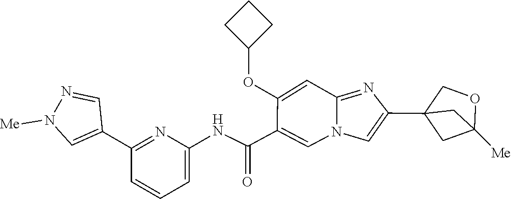 [SMILES: Cn1cc(-c2cccc(NC(=O)c3cn4cc(C56COC(C)(C5)C6)nc4cc3OC3CCC3)n2)cn1] Method B: RNH₂: 6-(1-methyl-1H-pyrazol-4-yl)pyridin-2-amine Yield: 4.2 mg LCMS m/z = 485.2 [M + H]⁺. |
| 521 | 7-Cyclobutoxy-2-(1-methyl-2-oxabicyclo[2.1.1]hexan-4-yl)-N-(1- (trifluoromethyl)-1H-pyrazol-3-yl)imidazo[1,2-a]pyridine-6-carboxamide 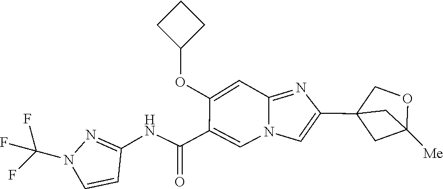 [SMILES: CC12CC(c3cn4cc(C(=O)Nc5ccn(C(F)(F)F)n5)c(OC5CCC5)cc4n3)(CO1)C2] Method C: RNH₂: 1-(trifluoromethyl)-1H-pyrazol-3-amine Yield: 6.1 mg (8.7%) LCMS m/z = 462.0 [M + H]⁺. |
| 522 | 7-Cyclobutoxy-2-(1-methyl-2-oxabicyclo[2.1.1]hexan-4-yl)-N-(1-(2- methylpyridin-4-yl)-1H-pyrazol-3-yl)imidazo[1,2-a]pyridine-6- carboxamide 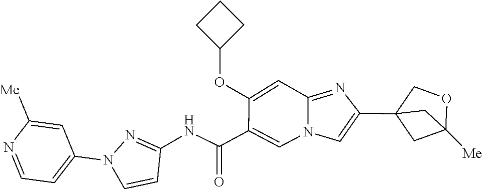 [SMILES: Cc1cc(-n2ccc(NC(=O)c3cn4cc(C56COC(C)(C5)C6)nc4cc3OC3CCC3)n2)ccn1] Method A: RNH₂: 1-(2-methylpyridin-4-yl)-1H-pyrazol-3-amine Yield: 8.4 mg LCMS m/z = 485.2 [M + H]⁺. |
| 523 | 7-Cyclobutoxy-N-(1-(difluoromethyl)-1H-pyrazol-3-yl)-2-(1-methyl-2- oxabicyclo[2.1.1]hexan-4-yl)imidazo[1,2-a]pyridine-6-carboxamide 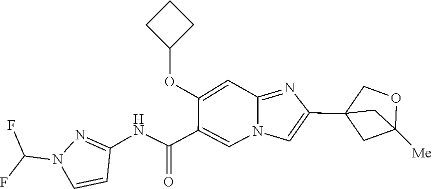 [SMILES: CC12CC(c3cn4cc(C(=O)Nc5ccn(C(F)F)n5)c(OC5CCC5)cc4n3)(CO1)C2] Method A: RNH₂: 1-(difluoromethyl)-1H-pyrazol-3-amine Yield: 8.3 mg LCMS m/z = 444.2 [M + H]⁺. |
| 524 | 7-Cyclobutoxy-N-(1-cyclobutyl-1H-pyrazol-3-yl)-2-(1-methyl-2- oxabicyclo[2.1.1]hexan-4-yl)imidazo[1,2-a]pyridine-6-carboxamide 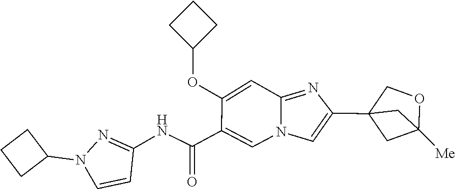 [SMILES: CC12CC(c3cn4cc(C(=O)Nc5ccn(C6CCC6)n5)c(OC5CCC5)cc4n3)(CO1)C2] Method A: RNH₂: 1-(cyclobutyl)-1H-pyrazole-3-amine Yield: 4.2 mg LCMS m/z = 448.2 [M + H]⁺. |
| 525 | 7-Cyclobutoxy-N-(1-(cyclopropylmethyl)-1H-pyrazol-3-yl)-2-(1-methyl- 2-oxabicyclo[2.1.1 ]hexan-4-yl)imidazo[1,2-a]pyridine-6-carboxamide 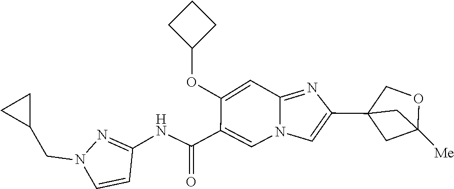 [SMILES: CC12CC(c3cn4cc(C(=O)Nc5ccn(CC6CC6)n5)c(OC5CCC5)cc4n3)(CO1)C2] Method A: RNH₂: 1-(cyclopropylmethyl)-1H-pyrazol-3-amine Yield: 7.1 mg LCMS m/z = 448.2 [M + H]⁺. |
| 526 | 7-Cyclobutoxy-N-(2-methoxypyridin-3-yl)-2-(1-methyl-2-oxa- bicyclo[2.1.1]hexan-4-yl)imidazo[1,2-a]pyridine-6-carboxamide 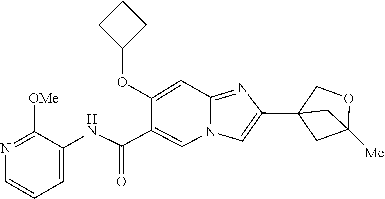 [SMILES: COc1ncccc1NC(=O)c1cn2cc(C34COC(C)(C3)C4)nc2cc1OC1CCC1] Method B: RNH₂: 2-methoxypyridin-3-amine Yield: 7.2 mg LCMS m/z = 435.2 [M + H]⁺. |
| 527 | N-(1-(cyanomethyl)-1H-pyrazol-3-yl)-7-cyclobutoxy-2-(1-methyl-2- oxabicyclo[2.1.1]hexan-4-yl)imidazo[1,2-a]pyridine-6-carboxamide 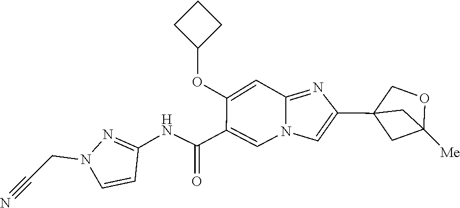 [SMILES: CC12CC(c3cn4cc(C(=O)Nc5ccn(CC#N)n5)c(OC5CCC5)cc4n3)(CO1)C2] Method A: RNH₂: 1-(cyanomethyl)-1H-pyrazol-3-amine Yield: 7 mg LCMS m/z = 433.2 [M + H]⁺. |
| 528 | 7-Cyclobutoxy-N-(1-ethyl-1H-pyrazol-3-yl)-2-(1-methyl-2- oxabicyclo[2.1.1]hexan-4-yl)imidazo[1,2-a]pyridine-6-carboxamide 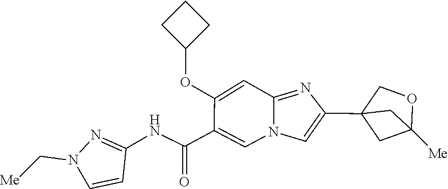 [SMILES: CCn1ccc(NC(=O)c2cn3cc(C45COC(C)(C4)C5)nc3cc2OC2CCC2)n1] Method A: RNH₂: 1-ethyl-1H-pyrazol-3-amine. Yield: 6.8 mg LCMS m/z = 422.2 [M + H]⁺. |
| 529 | 7-Cyclobutoxy-N-(6-methoxypyridin-2-yl)-2-(1-methyl-2- oxabicyclo[2.1.1]hexan-4-yl)imidazo[1,2-a]pyridine-6-carboxamide 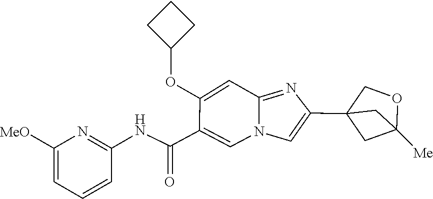 [SMILES: COc1cccc(NC(=O)c2cn3cc(C45COC(C)(C4)C5)nc3cc2OC2CCC2)n1] Method B: RNH₂: 6-methoxy-2-aminopyridine. Yield: 5.4 mg LCMS m/z = 435.0 [M + H]⁺. |
| 530 | 7-Cyclobutoxy-N-(1-(2,2-difluoroethyl)-1H-pyrazol-3-yl)-2-(1-methyl-2- oxabicyclo[2.1.1]hexan-4-yl)imidazo[1,2-a]pyridine-6-carboxamide 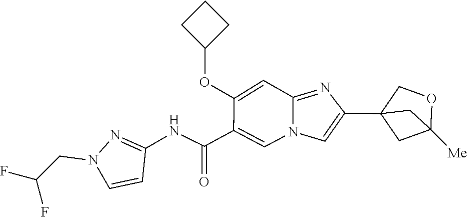 [SMILES: CC12CC(c3cn4cc(C(=O)Nc5ccn(CC(F)F)n5)c(OC5CCC5)cc4n3)(CO1)C2] Method A: RNH₂: 1-(2,2-difluoroethyl)-1H-pyrazol-3-amine Yield: 7 mg LCMS m/z = 458.2 [M + H]⁺. |
| 531 | 7-Cyclobutoxy-2-(1-methyl-2-oxabicyclo[2.2.1]heptan-4-yl)-N-(1-(2- methylpyridin-4-yl)-1H-pyrazol-3-yl)imidazo[1,2-a]pyridine-6- carboxamide 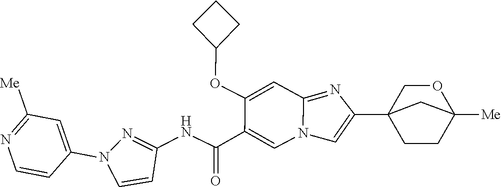 [SMILES: Cc1cc(-n2ccc(NC(=O)c3cn4cc(C56CCC(C)(C5)OC6)nc4cc3OC3CCC3)n2)ccn1] Method A: RNH₂: 1-(2-methylpyridin-4-yl)-1H-pyrazol-3-amine Yield: 9.6 mg LCMS m/z = 499.2 [M + H]⁺. |
| 532 | N-(1-(cyanomethyl)-1H-pyrazol-3-yl)-7-cyclobutoxy-2-(1-methyl-2- oxabicyclo[2.2.1]heptan-4-yl)imidazo[1,2-a]pyridine-6-carboxamide 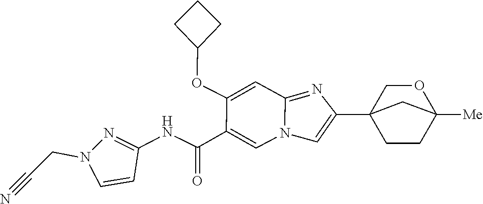 [SMILES: CC12CCC(c3cn4cc(C(=O)Nc5ccn(CC#N)n5)c(OC5CCC5)cc4n3)(CO1)C2] Method A: RNH₂: 1-(cyanomethyl)-1H-pyrazol-3-amine. Yield: 7.7 mg LCMS m/z = 447.2 [M + H]⁺. ¹H NMR(DMSO-d₆, 500 MHz) δ: 1.35 (s, 3H), 1.64-1.74 (m, 2H), 1.75-1.84 (m, 3H), 1.82-1.94 (m, 2H), 1.98-2.05 (m, 1H), 2.09-2.20 (m, 2H), 2.52-2.56 (m, 2H), 3.72 (d, 1H), 3.89 (dd, 1H), 4.89 (p, 1H), 5.42 (s, 2H), 6.70 (d, 1H), 6.81 (s, 1H), 7.68 (s, 1H), 7.78 (d, 1H), 8.90 (s, 1H), 10.53 (s, 1H). |
| 533 | 7-Cyclobutoxy-N-(1-ethyl-1H-pyrazol-3-yl)-2-(1-methyl-2- oxabicyclo[2.2.1]heptan-4-yl)imidazo[1,2-a]pyridine-6-carboxamide 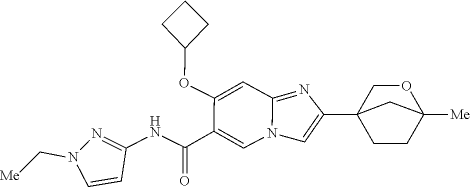 [SMILES: CCn1ccc(NC(=O)c2cn3cc(C45CCC(C)(C4)OC5)nc3cc2OC2CCC2)n1] Method A: RNH₂: 1-ethyl-1H-pyrazol-3-amine. Yield: 7.3 mg LCMS m/z = 436.2 [M + H]⁺. |
| 534 | 7-Cyclobutoxy-N-(1-(cyclopropylmethyl)-1H-pyrazol-3-yl)-2-(1-methyl- 2-oxabicyclo[2.2.1]heptan-4-yl)imidazo[1,2-a]pyridine-6-carboxamide 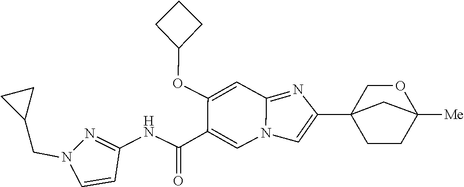 [SMILES: CC12CCC(c3cn4cc(C(=O)Nc5ccn(CC6CC6)n5)c(OC5CCC5)cc4n3)(CO1)C2] Method A: RNH₂: 1-cyclopropylmethyl-1H-pyrazol-3-amine Yield: 13.4 mg LCMS m/z = 462.2 [M + H]⁺. ¹H NMR(CDCl₃, 400 MHz) δ: 0.32-0.42 (m, 2H), 0.61-0.71 (m, 2H), 1.23-1.34 (m, 1H), 1.48 (s, 3H), 1.80-1.86 (m, 2H), 1.87-1.95 (m, 2H), 1.96-2.03 (m, 2H), 2.04-2.21 (m, 2H), 2.32-2.46 (m, 2H), 2.59-2.71 (m, 2H), 3.91 (d, 2H), 3.96 (d, 1H), 4.09 (dd, 1H), 4.85 (p, 1H), 6.72-6.84 (m, 2H), 7.32 (s, 1H), 7.45 (d, 1H), 9.00 (s, 1H), 10.20 (s, 1H). |
| 535 | 7-Cyclobutoxy-N-(1-ethyl-2-oxo-1,2-dihydropyridin-3-yl)-2-(1-methyl-2- oxabicyclo[2.2.1]heptan-4-yl)imidazo[1,2-a]pyridine-6-carboxamide 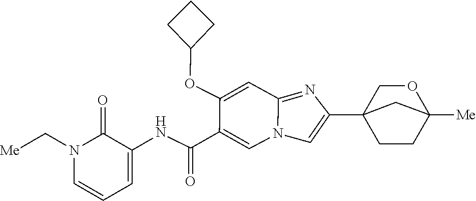 [SMILES: CCn1cccc(NC(=O)c2cn3cc(C45CCC(C)(C4)OC5)nc3cc2OC2CCC2)c1=O] Method B: RNH₂: 3-amino-1-ethylpyridin-2(1H)-one. Yield: 14.1 mg LCMS m/z = 463.2 [M + H]⁺. ¹H NMR(DMSO-d₆ + CCl₄, 400 MHz) δ: 1.35 (t, 3H), 1.39 (s, 3H), 1.70-1.80 (m, 3H), 1.80-1.88 (m, 2H), 1.89-2.13 (m, 3H), 2.56-2.70 (m, 4H), 3.73 (d, 1H), 3.77 (q, 1H), 3.92 (dd, 1H), 4.04-4.07 (m, 1H), 4.97 (p, 1H), 6.24 (t, 1H), 6.77 (s, 1H), 7.30 (dd, 1H), 7.61 (s, 1H), 8.48 (dd, 1H), 9.13 (s, 1H), 10.75 (s, 1H). |
| 536 | 7-Cyclobutoxy-N-(2-(difluoromethoxy)pyridin-3-yl)-2-(1-methyl-2- oxabicyclo[2.2.1]heptan-4-yl)imidazo[1,2-a]pyridine-6-carboxamide 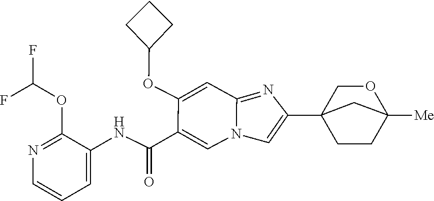 [SMILES: CC12CCC(c3cn4cc(C(=O)Nc5cccnc5OC(F)F)c(OC5CCC5)cc4n3)(CO1)C2] Method B: RNH₂: 2-(difluoromethoxy)pyridin-3-amine. Yield: 15.1 mg LCMS m/z = 485.2 [M + H]⁺. ¹H NMR(DMSO-d₆ + CCl₄, 400 MHz) δ: 1.39 (s, 3H), 1.72-1.87 (m, 5H), 1.90-2.00 (m, 2H), 2.02-2.14 (m, 1H), 2.33-2.44 (m, 2H), 2.60-2.73 (m, 2H), 3.74 (d, 1H), 3.93 (dd, 1H), 5.02 (p, 1H), 6.87 (s, 1H), 7.24 (dd, 1H), 7.53-7.96 (m, 3H), 8.95 (dd, 1H), 9.19 (s, 1H), 10.06 (s, 1H). |
| 537 | 7-Cyclobutoxy-N-(1-(2-fluoroethyl)-1H-pyrazol-3-yl)-2-(1-methyl-2- oxabicyclo[2.2.1]heptan-4-yl)imidazo[1,2-a]pyridine-6-carboxamide 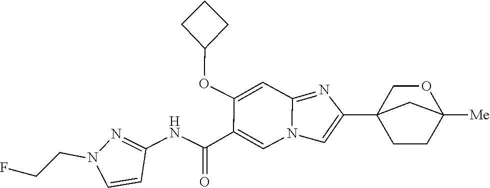 [SMILES: CC12CCC(c3cn4cc(C(=O)Nc5ccn(CCF)n5)c(OC5CCC5)cc4n3)(CO1)C2] Method B: RNH₂: 1-(2-fluoroethyl)-1H-pyrazol-3-amine. Yield: 6.6 mg LCMS m/z = 454.2 [M + H]⁺. ¹H NMR(DMSO-d₆, 500 MHz) δ: 1.35 (s, 3H), 1.61-1.71 (m, 2H), 1.73-1.80 (m, 3H), 1.82-1.94 (m, 2H), 1.98-2.06 (m, 1H), 2.09-2.20 (m, 2H), 2.53-2.57 (m, 2H), 3.72 (d, 1H), 3.89 (dd, 1H), 4.32 (t, 1H), 4.38 (t, 1H), 4.67 (t, 1H), 4.77 (t, 1H), 4.90 (p, 1H), 6.62 (d, 1H), 6.81 (s, 1H), 7.69 (s, 2H), 8.92 (s, 1H), 10.41 (s, 1H). |
| 538 | 7-Cyclobutoxy-N-(1-(difluoromethyl)-2-oxo-1,2-dihydropyridin-3-yl)-2- (1-methyl-2-oxabicyclo[2.2.1]heptan-4-yl)imidazo[1,2-a]pyridine-6- carboxamide 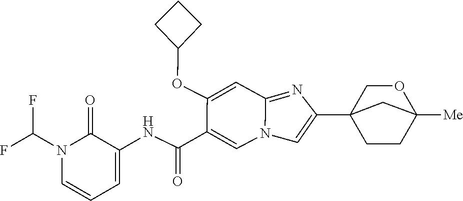 [SMILES: CC12CCC(c3cn4cc(C(=O)Nc5cccn(C(F)F)c5=O)c(OC5CCC5)cc4n3)(CO1)C2] Method B: RNH₂: 3-amino-1-(difluoromethyl)pyridin-2(1H)-one Yield: 13.9 mg LCMS m/z = 485.2 [M + H]⁺. ¹H NMR(DMSO-d₆, 500 MHz) δ: 1.35 (s, 3H), 1.63-1.74 (m, 2H), 1.74-1.83 (m, 3H), 1.83-1.94 (m, 2H), 1.98-2.04 (m, 1H), 2.40-2.45 (m, 2H), 2.55-2.59 (m, 2H), 3.73 (d, 1H), 3.90 (dd, 1H), 5.01 (p, 1H), 6.54 (t, 1H), 6.90 (s, 1H), 7.57 (dd, 1H), 7.79 (s, 1H), 7.99 (t, 1H), 8.54 (dd, 1H), 9.19 (s, 1H), 10.68 (s, 1H). |
| 539 | 7-Cyclobutoxy-N-(2-isopropoxypyridin-3-yl)-2-(1-methyl-2- oxabicyclo[2.2.1]heptan-4-yl)imidazo[1,2-a]pyridine-6-carboxamide 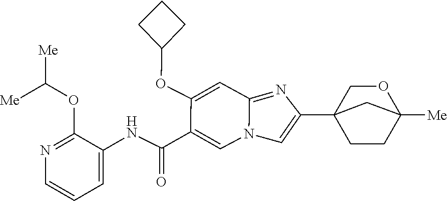 [SMILES: CC(C)Oc1ncccc1NC(=O)c1cn2cc(C34CCC(C)(C3)OC4)nc2cc1OC1CCC1] Method B: RNH₂: 2-isopropoxypyridin-3-amine. Yield: 13.9 mg LCMS m/z = 477.2 [M + H]⁺. ¹H NMR(CDCl₃, 400 MHz) δ: 1.43 (d, 6H), 1.49 (s, 3H), 1.78-1.90 (m, 3H), 1.95-2.02 (m, 2H), 2.03-2.21 (m, 3H), 2.34-2.48 (m, 2H), 2.59-2.70 (m, 2H), 3.97 (d, 1H), 4.10 (dd, 1H), 4.89 (p, 1H), 5.51 (hept, 1H), 6.85 (s, 1H), 6.93 (dd, 1H), 7.33 (d, 1H), 7.90 (dd, 1H), 8.73 (dd, 1H), 8.96 (s, 1H), 9.83 (s, 1H). |
| 540 | N-([1,2,4]triazolo[4,3-a]pyridin-8-yl)-7-cyclobutoxy-2-(1-methyl-2- oxabicyclo[2.2.1]heptan-4-yl)imidazo[1,2-a]pyridine-6-carboxamide 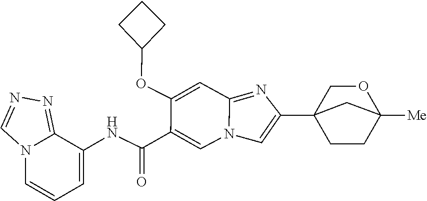 [SMILES: CC12CCC(c3cn4cc(C(=O)Nc5cccn6cnnc56)c(OC5CCC5)cc4n3)(CO1)C2] Method B: RNH₂: [1,2,4]triazolo[4,3-a]pyridin-8-amine Yield: 6.1 mg LCMS m/z = 459.2 [M + H]⁺. |
| 541 | 7-Cyclobutoxy-N-(6-methoxypyridin-2-yl)-2-(1-methyl-2- oxabicyclo[2.2.1]heptan-4-yl)imidazo[1,2-a]pyridine-6-carboxamide 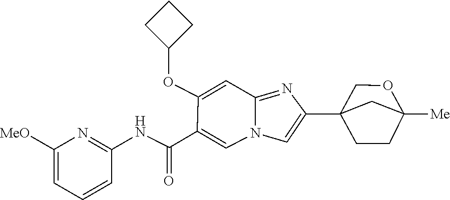 [SMILES: COc1cccc(NC(=O)c2cn3cc(C45CCC(C)(C4)OC5)nc3cc2OC2CCC2)n1] Method B: RNH₂: 6-methoxy-2-aminopyridine. Yield: 11.7 mg LCMS m/z = 449.2 [M + H]⁺. ¹H NMR(CDCl₃, 400 MHz) δ: 1.49 (s, 3H), 1.81-1.85 (m, 1H), 1.86-1.89 (m, 1H), 1.91-1.94 (m, 1H), 1.98-2.02 (m, 1H), 2.02-2.13 (m, 3H), 2.13-2.21 (m, 1H), 2.38-2.52 (m, 2H), 2.68 (tdd, 2H), 3.90 (s, 3H), 3.97 (d, 1H), 4.10 (dd, 1H), 4.91 (p, 1H), 6.52-6.59 (m, 1H), 6.84 (s, 1H), 7.34 (s, 1H), 7.64 (t, 1H), 7.89 (d, 1H), 9.02 (s, 1H), 10.49 (s, 1H). |
| 542 | 7-Cyclobutoxy-N-(6-(1-methyl-1H-pyrazol-4-yl)pyridin-2-yl)-2-(1- methyl-2-oxabicyclo[2.2.1]heptan-4-yl)imidazo[1,2-a]pyridine-6- carboxamide 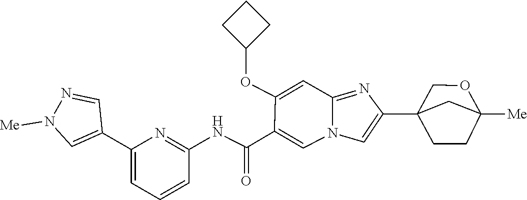 [SMILES: Cn1cc(-c2cccc(NC(=O)c3cn4cc(C56CCC(C)(C5)OC6)nc4cc3OC3CCC3)n2)cn1] Method B: RNH₂: 1-methyl-1H-pyrazol-3-amine. Yield: 4.2 mg LCMS m/z = 499.2 [M + H]⁺. |
| 543 | 7-Cyclobutoxy-N-(1-(2,2-difluoroethyl)-1H-pyrazol-3-yl)-2-(1-methyl-2- oxabicyclo[2.2.1]heptan-4-yl)imidazo[1,2-a]pyridine-6-carboxamide 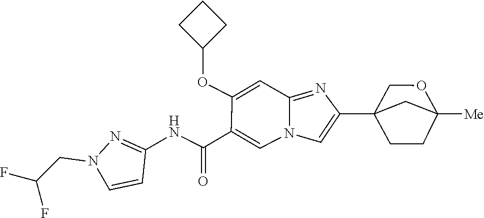 [SMILES: CC12CCC(c3cn4cc(C(=O)Nc5ccn(CC(F)F)n5)c(OC5CCC5)cc4n3)(CO1)C2] Method A: RNH₂: 1-(2,2-difluoroethyl)-1H-pyrazol-3-amine Yield: 7.3 mg LCMS m/z = 472.2 [M + H]⁺. |

### Example 544: 2-(2-Oxabicyclo[2.1.1]hexan-4-yl)-N-(6-(difluoromethyl)pyridin-2-yl)-7-isopropoxyimidazo[1,2-a]pyridine-6-carboxamide

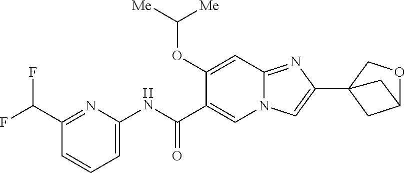 [SMILES: CC(C)Oc1cc2nc(C34COC(C3)C4)cn2cc1C(=O)Nc1cccc(C(F)F)n1]

HATU (151 mg, 0.397 mmol), DIPEA (128 mg, 0.992 mmol) and 6-(difluoromethyl)pyridin-2-amine (52.4 mg, 0.364 mmol) were added to a solution of 2-(2-oxabicyclo[2.1.1]hexan-4-yl)-7-isopropoxyimidazo[1,2-a]pyridine-6-carboxylic acid (Preparation 307, 100 mg, 0.331 mmol) in DMF (1 mL) and the mixture stirred at rt for 18 h. The reaction mixture was diluted with H₂O (20 mL) and extracted with EtOAc (3×25 mL). The combined organics were washed with H₂O (20 mL), brine (20 mL), dried (Na₂SO₄) and evaporated to dryness in vacuo. The residue was purified by prep-HPLC-L (gradient: 60-100%) to give 2-(2-oxabicyclo[2.1.1]hexan-4-yl)-N-(6-(difluoromethyl)pyridin-2-yl)-7-isopropoxyimidazo[1,2-a]pyridine-6-carboxamide (32.8 mg, 23%). LCMS m/z=429.2 [M+H]⁺, ¹H NMR (400 MHz, DMSO-d₆) δ: 10.82 (s, 1H), 9.07 (s, 1H), 8.32 (d, 1H), 8.03 (t, 1H), 7.78 (s, 1H), 7.43 (d, 1H), 7.13 (s, 1H), 6.85 (t, 1H), 4.95-4.83 (m, 1H), 4.54 (s, 1H), 3.80 (s, 2H), 2.09-2.05 (m, 2H), 1.73-1.68 (m, 2H), 1.40 (d, 6H).

### Example 545-547

The title compounds were prepared using an analogous method to that described for Example 544.

| Example | Name/Structure/Data |
|---|---|
| 545 | 2-(2-Oxabicyclo[2.1.1]hexan-4-yl)-7-isopropoxy-N-(2-methoxypyridin-3- |
| | yl)imidazo[1,2-a]pyridine-6-carboxamide |
| | 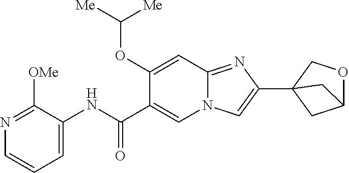 [SMILES: COc1ncccc1NC(=O)c1cn2cc(C34COC(C3)C4)nc2cc1OC(C)C] |
| | LCMS m/z = 409.2 [M + H]⁺, ¹H NMR (400 MHz, DMSO-d₆) δ: 10.24 (s, |
| | 1H), 9.21 (s, 1H), 8.72 (dd, 1H), 7.91 (dd, 1H), 7.86 (s, 1H), 7.22 (s, 1H), |
| | 7.04 (dd, 1H), 5.10-4.97 (m, 1H), 4.58 (s, 1H), 4.00 (s, 3H), 3.84 (s, 2H), |
| | 2.15-2.06 (m, 2H), 1.79-1.71 (m, 2H), 1.48 (d, 6H). |
| 546 | 2-(2-Oxabicyclo[2.1.1]hexan-4-yl)-7-isopropoxy-N-(1-methyl-1H-pyrazol- |
| | 3-yl)imidazo[1,2-a]pyridine-6-carboxamide |
| | 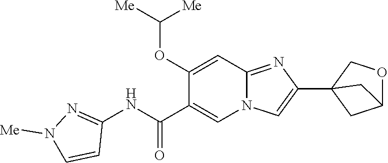 [SMILES: CC(C)Oc1cc2nc(C34COC(C3)C4)cn2cc1C(=O)Nc1ccn(C)n1] |
| | LCMS m/z = 382.4 [M + H]⁺, ¹H NMR (400 MHz, DMSO-d₆) δ: 10.34 (s, |
| | 1H), 8.98 (s, 1H), 7.76 (s, 1H), 7.58 (d, 1H), 7.08 (s, 1H), 6.54 (d, 1H), |
| | 4.94-4.80 (m, 1H), 4.54 (s, 1H), 3.80 (s, 2H), 3.73 (s, 3H), 2.09-2.04 (m, |
| | 2H), 1.73-1.67 (m, 2H), 1.38 (d, 6H). |
| 547 | 2-(2-Oxabicyclo[2.1.1]hexan-4-yl)-N-(1-(difluoromethyl)-2-oxo-1,2- |
| | dihydropyridin-3-yl)-7-isopropoxyimidazo[1,2-a]pyridine-6-carboxamide |
| | 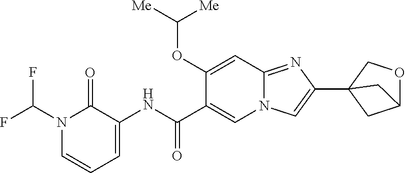 [SMILES: CC(C)Oc1cc2nc(C34COC(C3)C4)cn2cc1C(=O)Nc1cccn(C(F)F)c1=O] |
| | LCMS m/z = 445.4 [M + H]⁺, ¹H NMR (400 MHz, DMSO-d₆) δ: 10.63 (s, |
| | 1H), 9.33 (s, 1H), 8.53 (d, 1H), 8.20-7.83 (m, 2H), 7.60 (d, 1H), 7.30 (s, |
| | 1H), 6.56 (t, 1H), 5.13 (p, 1H), 4.62 (s, 1H), 3.88 (s, 2H), 2.23 (d, 2H), |
| | 1.83-1.78 (m, 2H), 1.53 (d, 6H). |

### Example-548: 8-Fluoro-7-isopropoxy-N-(6-methoxypyridin-2-yl)-2-(1-methyl-2-oxabicyclo[2.1.1]hexan-4-yl)imidazo[1,2-a]pyridine-6-carboxamide trifluoroacetate

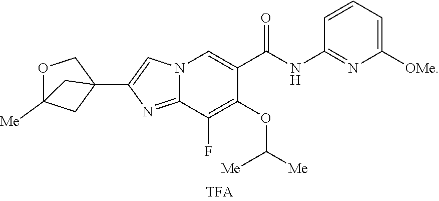 [SMILES: COc1cccc(NC(=O)c2cn3cc(C45COC(C)(C4)C5)nc3c(F)c2OC(C)C)n1]

6-Methoxypyridin-2-amine (14.5 mg, 0.117 mmol) was added to a solution of 8-fluoro-7-isopropoxy-2-(1-methyl-2-oxabicyclo[2.1.1]hexan-4-yl)imidazo[1,2-a]pyridine-6-carboxylic acid (Preparation 127, 30 mg, 0.090 mmol), HATU (47.9 mg, 0.126 mmol) and DIPEA (37.0 mg, 0.286 mmol) in DMF (0.9 mL) and the reaction stirred at rt overnight. The mixture was concentrated in vacuo and the crude purified by prep-HPLC-D to afford 8-fluoro-7-isopropoxy-N-(6-methoxypyridin-2-yl)-2-(1-methyl-2-oxabicyclo[2.1.1]hexan-4-yl)imidazo[1,2-a]pyridine-6-carboxamide trifluoroacetate. LCMS m/z=441.0 [M+H]⁺; ¹H NMR (500 MHz, DMSO-d₆) δ: 1.41 (d, 3H), 1.43 (s, 2H), 1.79 (dd, 1H), 2.04 (dd, 1H), 3.85 (s, 3H), 3.90 (s, 2H), 4.66-4.79 (m, 1H), 6.55-6.70 (m, 1H), 7.72-7.87 (m, 2H), 7.93-8.04 (m, 1H), 9.02 (s, 1H), 10.55 (s, 1H).

### Example 549-560

The compounds in the following table were prepared from the appropriate carboxylic acid (RCO₂H) and appropriate amine (RNH₂) following a similar procedure to that described in Example 548. Compounds purified using the prep-HPLC method noted in the table.

| Ex No | Name/Structure | Data |
|---|---|---|
| 549 | 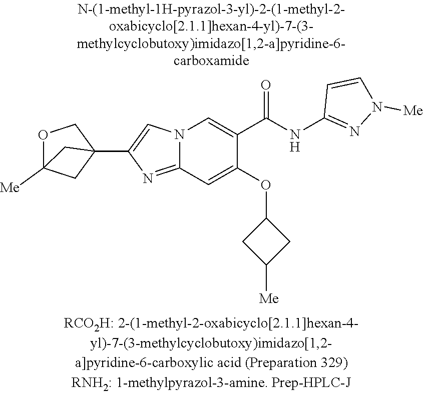 [SMILES: CC1CC(Oc2cc3nc(C45COC(C)(C4)C5)cn3cc2C(=O)Nc2ccn(C)n2)C1] | LCMS m/z = 422.3 [M + H]⁺ ¹H NMR (500 MHz, MeOD-d₄) δ 1.27 (d, 3H), 1.48-1.52 (m, 3H), 1.87 (dd, 2H), 2.09 (dd, 2H), 2.32 (td, 1H), 2.47-2.67 (m, 2H), 2.77-2.89 (m, 1H), 3.34 (s, 1H), 3.81-3.87 (m, 3H), 3.99 (s, 2H), 5.13 (t, 1H), 6.65-6.74 (m, 2H), 7.52 (d, 1H), 7.68 (s, 1H), 9.02 (s, 1H) |
| 550 | 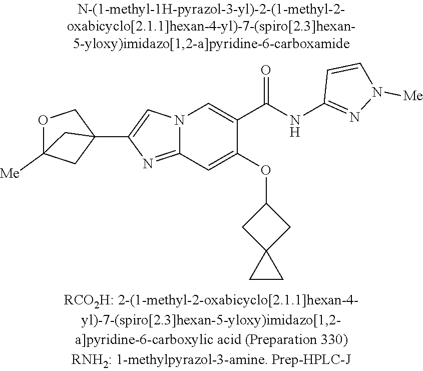 [SMILES: Cn1ccc(NC(=O)c2cn3cc(C45COC(C)(C4)C5)nc3cc2OC2CC3(CC3)C2)n1] | LCMS m/z = 434.2 [M + H]⁺ ¹H NMR (500 MHz, MeOH-d₄) δ 0.60-0.66 (m, 2H), 0.53-0.58 (m, 2H) 1.50 (s, 3H), 1.86 (dd, 2H), 2.09 (dd, 2H), 2.64 (d, 4H), 3.84 (s, 3H), 4.00 (s, 2H), 5.18-5.25 (m, 1H), 6.68 (d, 1H), 6.79 (s, 1H), 7.52 (d, 1H), 7.69 (s, 1H), 9.03 (s, 1H). |
| 551 | 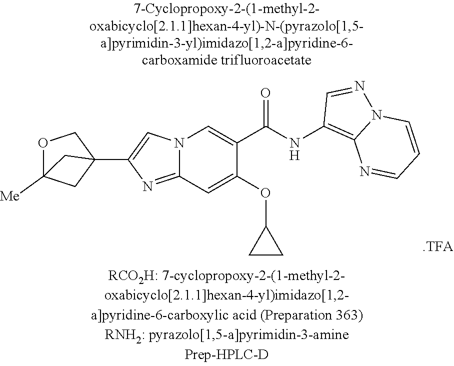 | LCMS m/z = 431.3 [M + H]⁺ ¹H NMR (500 MHz, DMSO-d₆) δ: 0.97- 1.02 (m, 2H), 1.02- 1.07 (m, 2H), 1.44 (s, 4H), 1.74-1.81 (m, 2H), 1.97-2.05 (m, 2H), 3.89 (s, 2H), 7.04-7.10 (m, 1H), 7.40 (s, 1H), 7.85 (s, 1H), 8.56-8.59 (m, 1H), 8.72 (s, 1H), 9.10 (d, 1H), 9.14 (s, 1H), 10.16 (s, 1H). |
| 552 | 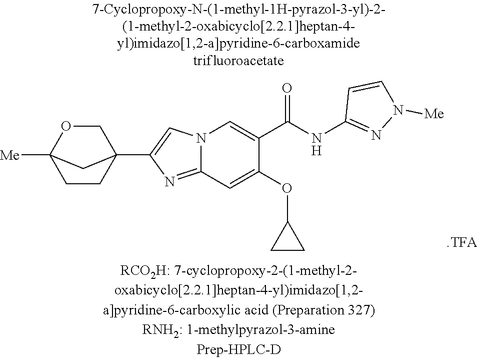 [SMILES: Cn1ccc(NC(=O)c2cn3cc(C45CCC(C)(C4)OC5)nc3cc2OC2CC2)n1] | LCMS m/z = 408.2 [M + H]⁺ ¹H NMR (500 MHz, MeOH-d₄) δ : 1.03- 1.13 (m, 4H), 1.51 (s, 3H), 1.85-2.01 (m, 2H), 2.07 (s, 2H), 2.20 (td, 2H), 3.86 (s, 3H), 3.98-4.01 (m, 1H), 4.03-4.07 (m, 1H), 4.25-4.32 (m, 1H), 6.68 (d, 1H), 7.55 (d, 1H), 7.58 (s, 1H), 8.00 (s, 1H), 9.14 (s, 1H) |
| 553 | 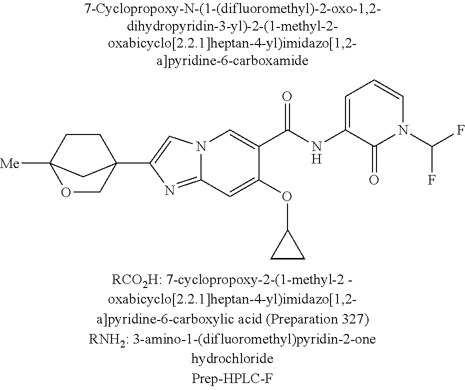 [SMILES: CC12CCC(c3cn4cc(C(=O)Nc5cccn(C(F)F)c5=O)c(OC5CC5)cc4n3)(CO1)C2] | LCMS m/z = 471.2 [M + H]⁺; ¹H NMR (500 MHz, DMSO-d₆) δ: 0.95-1.00 (m, 2H), 1.07-1.12 (m, 2H), 1.39 (s, 3H), 1.66-1.74 (m, 1H), 1.77-1.88 (m, 3H), 1.92-2.00 (m, 1H), 2.03-2.12 (m, 1H), 3.78 (d, 1H), 3.94 (dd, 1H), 4.26 (br s, 1H), 6.51-6.59 (m, 1H), 7.43 (s, 1H), 7.60 (dd, 1H), 7.83-8.16 (m, 2H), 8.51 (dd, 1H), 9.24 (br s, 1H), 10.47 (s, 1H) |
| 554 | 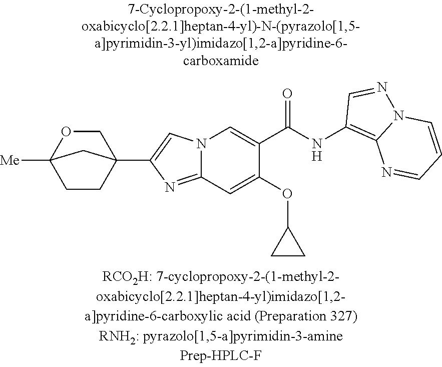 | LCMS m/z = 445.2 [M + H]⁺; ¹H NMR (500 MHz, DMSO-d₆) δ: 0.97-1.02 (m, 2H), 1.02-1.07 (m, 2H), 1.44 (s, 3H), 1.65-1.74 (m, 1H), 1.77-1.81 (m, 1H), 1.82-1.86 (m, 2H), 1.91-2.00 (m, 1H), 2.04-2.14 (m, 1H), 3.77 (d, 1H), 3.92-3.98 (m, 1H), 4.20-4.28 (m, 1H), 7.06-7.10 (m, 1H), 7.41 (s, 1H), 7.84 (s, 1H), 8.56-8.59 (m, 1H), 8.72 (s, 1H), 9.10 (d, 1H), 9.13 (s, 1H), 10.16 (s, 1H). |
| 555 | 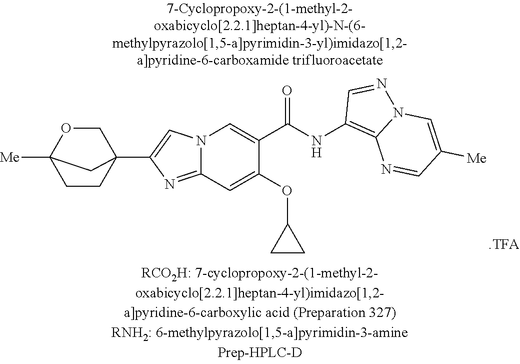 [SMILES: Cc1cnc2c(NC(=O)c3cn4cc(C56CCC(C)(C5)OC6)nc4cc3OC3CC3)cnn2c1] | LCMS m/z = 459.3 [M + H]⁺ ¹H NMR (500 MHz, MeOH-d₄) δ: 1.08- 1.15 (m, 2H), 1.18- 1.25 (m, 2H), 1.51 (s, 3H), 1.84-2.02 (m, 2H), 2.08 (s, 2H), 2.16-2.27 (m, 2H), 2.44 (s, 3H), 3.98-4.03 (m, 1H), 4.04-4.10 (m, 1H), 4.34-4.41 (m, 1H), 7.62 (s, 1H), 8.05 (s, 1H), 8.49 (d, 1H), 8.67 (s, 1H), 8.72 (s, 1H), 9.29 (s, 1H) |
| 556 | 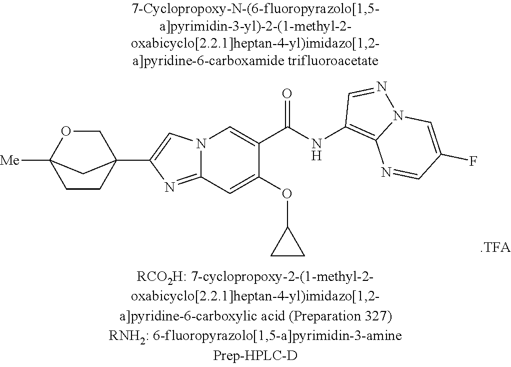 | LCMS m/z = 463.2 [M + H]⁺ ¹H NMR (500 MHz, MeOH-d₄) δ: 1.08- 1.16 (m, 2H), 1.17- 1.25 (m, 2H), 1.51 (s, 3H), 1.86-2.01 (m, 2H), 2.08 (s, 2H), 2.16-2.28 (m, 2H), 3.98-4.03 (m, 1H), 4.03-4.08 (m, 1H), 4.34-4.42 (m, 1H), 7.61 (s, 1H), 8.05 (s, 1H), 8.69 (d, 1H), 8.78 (s, 1H), 9.09-9.14 (m, 1H), 9.29 (s, 1H) |
| 557 | 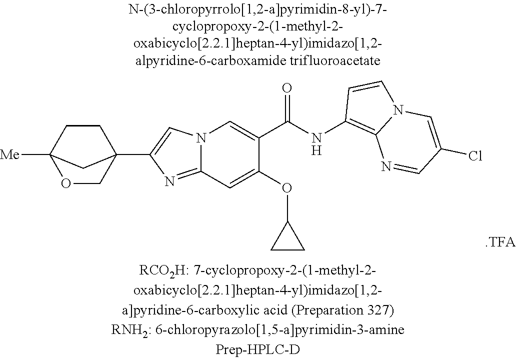 [SMILES: CC12CCC(c3cn4cc(C(=O)Nc5ccn6cc(Cl)cnc56)c(OC5CC5)cc4n3)(CO1)C2] | LCMS m/z = 479.1 [M + H]⁺ ¹HNMR (500 MHz, MeOH-d₄) δ: H NMR (500 MHz, MeOH-d₄) δ: 1.79-1.85 (m, 4H), 2.23 (s, 3H), 2.48-2.58 (m, 1H), 2.59-2.69 (m, 1H), 2.71-2.80 (m, 2H), 2.80-2.99 (m, 2H), 4.63-4.68 (m, 1H), 4.73-4.78 (m, 1H), 5.09-5.16 (m, 1H), 8.29 (s, 1H), 8.76-8.94 (m, 1H), 9.46 (d, 1H), 9.55 (s, 1H), 10.02 (s, 1H), 10.38 (s, 1H). |
| 558 | 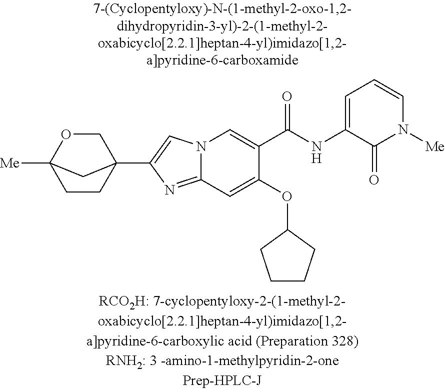 [SMILES: Cn1cccc(NC(=O)c2cn3cc(C45CCC(C)(C4)OC5)nc3cc2OC2CCCC2)c1=O] | LCMS m/z = 463.2 [M + H]⁺ ¹H NMR (500 MHz, MeOH-d₄) δ: 1.49 (s, 3H), 1.69-1.81 (m, 2H), 1.83-2.04 (m, 2H), 2.05 (s, 2H), 2.13-2.26 (m, 5H), 2.28-2.37 (m, 2H), 3.66 (s, 3H), 3.95-3.99 (m, 1H), 4.00-4.05 (m, 1H), 5.26-5.38 (m, 1H), 6.39 (t, 1H), 7.23 (s, 1H), 7.40 (dd, 1H), 7.99 (s, 1H), 8.59 (dd, 1H), 9.35 (s, 1H) |
| 559 | 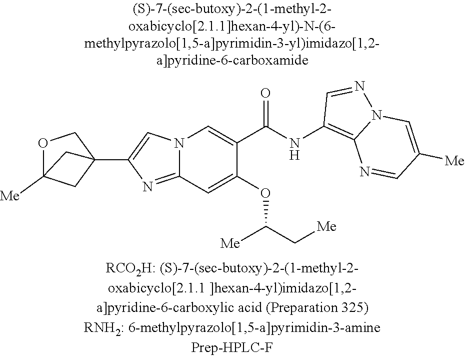 | LCMS m/z = 461.2 [M + H]⁺; ¹H NMR (400 MHz, CDCl₃) δ: 1.11 (t, 3H), 1.60 (d, 3H), 1.87-2.01 (m, 3H), 2.07-2.23 (m, 3H), 2.40 (s, 3H), 3.04 (q, 1H), 4.03 (s, 2H), 7.07 (s, 1H), 7.79 (s, 1H), 8.43 (d, 1H), 8.66 (s, 1H), 8.69 (s, 1H), 9.19 (s, 1H) |
| 560 | 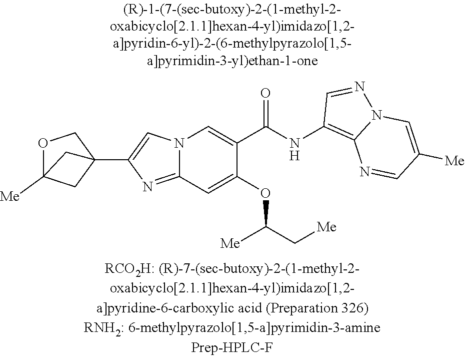 | LCMS m/z = 461.2 [M + H]⁺ ¹H NMR (400 MHz, CDCl₃) δ: 1.05-1.15 (m, 4H), 1.30 (t, 3H), 1.51 (s, 3H), 1.59 (d, 3H), 1.83-2.02 (m, 3H), 2.07-2.25 (m, 3H), 2.40 (s, 3H), 2.99-3.10 (m, 2H), 4.02 (s, 2H), 7.02 (s, 1H), 7.74 (s, 1H), 8.43 (s, 3H), 8.65 (s, 1H), 8.70 (s, 1H), 9.15 (s, 1H). |

### Example 561: 7-Cyclobutoxy-N-(1-methyl-1H-pyrazol-3-yl)-2-(1-methyl-2-oxabicyclo[2.2.1]heptan-4-yl)imidazo[1,2-a]pyridine-6-carboxamide

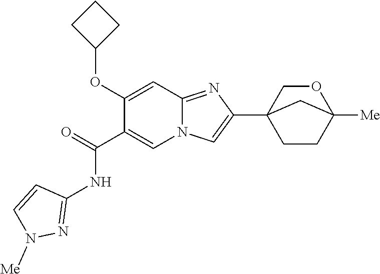 [SMILES: Cn1ccc(NC(=O)c2cn3cc(C45CCC(C)(C4)OC5)nc3cc2OC2CCC2)n1]

HATU (30.9 mg, 0.081 mmol) and DIPEA (29.9 mg, 0.231 mmol) were to a solution of 7-cyclobutoxy-2-(1-methyl-2-oxabicyclo[2.2.1]heptan-4-yl)imidazo[1,2-a]pyridine-6-carboxylic acid (Preparation 296, 40 mg, 0.077 mmol) and 1-methyl-1H-pyrazol-3-amine (18 mg, 0.185 mmol) in DMF (1.5 mL) and the mixture stirred at rt for 18 h. The mixture was evaporated to dryness in vacuo and the residue partitioned between EtOAc and H₂O. The combined organics were evaporated to dryness and the residue purified by normal phase chromatography (24 g, EtOAc/EtOH, I/O to 7/1) to afford 7-cyclobutoxy-N-(1-methyl-1H-pyrazol-3-yl)-2-(1-methyl-2-oxabicyclo[2.2.1]heptan-4-yl)imidazo[1,2-a]pyridine-6-carboxamide as a white solid (26 mg, 80%). LCMS m/z=422.2 [M+H]⁺; ¹H NMR (400 MHz, MeOH-d₄) δ: 1.48 (s, 3H), 1.78-2.24 (m, 9H), 2.34-2.49 (m, 2H), 2.58-2.77 (m, 2H), 3.86 (s, 3H), 3.93 (d, 1H), 4.05 (dd, 1H), 5.03 (quin, 1H), 6.70 (d, 1H), 6.80 (s, 1H), 7.54 (d, 1H), 7.68 (s, 1H), 9.03 (s, 1H).

### Example 562: 7-Isopropoxy-2-(1-methyl-2-oxabicyclo[2.1.1]hexan-4-yl)-N-(6-methylpyrazolo[1,5-a]pyrimidin-3-yl)imidazo[1,2-a]pyrimidine-6-carboxamide

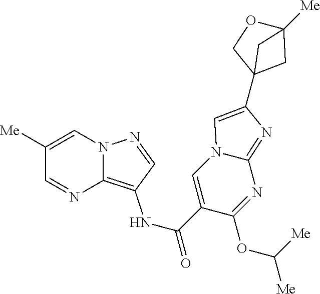 [SMILES: Cc1cnc2c(NC(=O)c3cn4cc(C56COC(C)(C5)C6)nc4nc3OC(C)C)cnn2c1]

T3P® (50 wt. % in EtOAc, 5.03 g, 7.90 mmol) was added to a mixture of 7-isopropoxy-2-(1-methyl-2-oxabicyclo[2.1.1]hexan-4-yl)imidazo[1,2-a]pyrimidine-6-carboxylic acid (Preparation 128, 501 mg, 1.58 mmol) and 6-methylpyrazolo[1,5-a]pyrimidin-3-amine hydrochloride (379 mg, 2.05 mmol) in pyridine (7.9 mL) at rt. The reaction mixture was stirred at rt overnight and diluted with EtOAc and H₂O and extracted with DCM (5×). The combined organics were dried (Na₂SO₄) and evaporated to dryness in vacuo. The residue was purified by silica gel chromatography (0-100% EtOAc/EtOH (3:1) in heptanes) to obtain 7-isopropoxy-2-(1-methyl-2-oxabicyclo[2.1.1]hexan-4-yl)-N-(6-methylpyrazolo[1,5-a]pyrimidin-3-yl)imidazo[1,2-a]pyrimidine-6-carboxamide (202 mg, 28.5%). LCMS m/z=448.2 [M+H]⁺; ¹H NMR (500 MHz, CDCl₃) δ: 1.55 (s, 3H), 1.66 (d, 7H) 1.98 (dd, 2H), 2.17 (br s, 2H), 2.42 (d, 4H), 4.10 (s, 2H), 5.78-5.96 (m, 1H), 7.31 (s, 1H), 8.34 (d, 1H), 8.42 (d, 1H), 8.83 (s, 1H), 9.26 (s, 1H), 10.41 (br s, 1H)

### Example 563: 2-(1-(Fluoromethyl)-2-oxabicyclo[2.1.1]hexan-4-yl)-7-isopropoxy-N-(pyrazolo[1,5-a]pyrimidin-3-yl)imidazo[1,2-a]pyrimidine-6-carboxamide

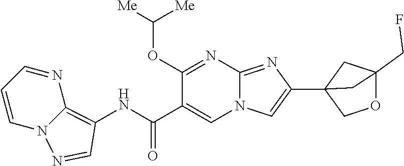 [SMILES: CC(C)Oc1nc2nc(C34COC(CF)(C3)C4)cn2cc1C(=O)Nc1cnn2cccnc12]

T3P® (50 wt. % in EtOAc, 712 mg, 1.12 mmol) was added to a mixture 2-(1-(fluoromethyl)-2-oxabicyclo[2.1.1]hexan-4-yl)-7-isopropoxyimidazo[1,2-a]pyrimidine-6-carboxylic acid (Preparation 311, 75 mg, 0.224 mmol) and pyrazolo[1,5-a]pyrimidin-3-amine (42 mg, 0.313 mmol) in pyridine (2 mL) at room temperature and the mixture stirred at rt for 2 h. The reaction was diluted with H₂O and extracted with EtOAc (3×). The combined organics were dried (MgSO₄) and evaporated to dryness in vacuo. The residue was purified by prep-HPLC-F (gradient: 5-95%) to afford 2-(1-(fluoromethyl)-2-oxabicyclo[2.1.1]hexan-4-yl)-7-isopropoxy-N-(pyrazolo[1,5-a]pyrimidin-3-yl)imidazo[1,2-a]pyrimidine-6-carboxamide (24.4 mg, 24%). LCMS m/z=452.2 [M+H]⁺; ¹H NMR (500 MHz, DMSO-d₆) δ: 1.57 (d, 6H), 1.81-1.91 (m, 2H), 2.12-2.22 (m, 2H), 3.97 (s, 2H), 4.71 (d, 2H), 5.56 (dt, 1H), 7.09 (dd, 1H), 7.78 (s, 1H), 8.53-8.62 (m, 1H), 8.73-8.82 (m, 1H), 9.07-9.17 (m, 1H), 9.48 (s, 1H), 10.35 (s, 1H).

### Example 564: N-(6-(difluoromethyl)pyridin-2-yl)-2-(1-(fluoromethyl)-2-oxabicyclo[2.1.1]hexan-4-yl)-7-isopropoxyimidazo[1,2-a]pyrimidine-6-carboxamide

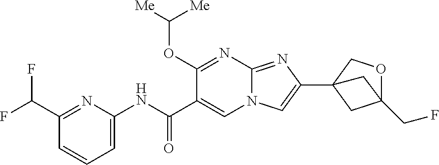 [SMILES: CC(C)Oc1nc2nc(C34COC(CF)(C3)C4)cn2cc1C(=O)Nc1cccc(C(F)F)n1]

T3P® (50 wt. % in EtOAc, 920 mg, 1.45 mmol) was added to a mixture 2-(1-(fluoromethyl)-2-oxabicyclo[2.1.1]hexan-4-yl)-7-isopropoxyimidazo[1,2-a]pyrimidine-6-carboxylic acid (Preparation 311, 97 mg, 0.289 mmol) and 6-(difluoromethyl)pyridin-2-amine (68 mg, 0.376 mmol) in pyridine (1.22 mL) and the mixture stirred at rt for 2 h. The reaction was diluted with H₂O and extracted with EtOAc (3×). The combined organics were washed with brine, dried (MgSO₄) and evaporated to dryness in vacuo. The residue was purified by prep-HPLC-F (gradient, 5-95%) to afford N-(6-(difluoromethyl)pyridin-2-yl)-2-(1-(fluoromethyl)-2-oxabicyclo[2.1.1]hexan-4-yl)-7-isopropoxyimidazo[1,2-a]pyrimidine-6-carboxamide (94.1 mg, 70%). LCMS m/z=462.2 [M+H]⁺; ¹H NMR (500 MHz, DMSO-d₆) δ: 1.45 (br d, 6H), 1.86-1.94 (m, 2H), 2.20 (br d, 2H), 3.99 (s, 2H), 4.66-4.79 (m, 2H), 5.44 (dt, 1H), 6.79-7.06 (m, 1H), 7.50 (d, 1H), 7.82 (s, 1H), 8.10 (t, 1H), 8.35 (br s, 1H), 9.40 (s, 1H), 10.89 (br s, 1H).

### Example 565: 2-(2-Oxabicyclo[2.2.1]heptan-4-yl)-7-isopropoxy-N-(pyrazolo[1,5-a]pyrimidin-3-yl)imidazo[1,2-a]pyrimidine-6-carboxamide

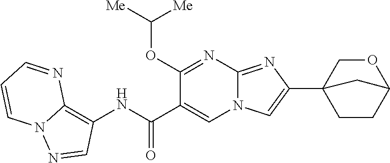 [SMILES: CC(C)Oc1nc2nc(C34CCC(C3)OC4)cn2cc1C(=O)Nc1cnn2cccnc12]

A mixture of 1-(2-oxabicyclo[2.2.1]heptan-4-yl)-2-bromoethan-1-one (Preparation 170, 16.5 mg, 0.075 mmol), 2-amino-4-isopropoxy-N-(pyrazolo[1,5-a]pyrimidin-3-yl)pyrimidine-5-carboxamide (Preparation 373, 15 mg, 0.048 mmol) and NaHCO₃ (14.4 mg, 0.171 mmol) in MeCN (0.6 mL) and toluene (0.4 mL) was heated at 90° C. overnight. The reaction mixture was partitioned between EtOAc and brine and the aqueous layer extracted with EtOAc. The combined organics were evaporated to dryness and the residue purified by column chromatography (12 g, 100% EtOAc) to afford 2-(2-oxabicyclo[2.2.1]heptan-4-yl)-7-isopropoxy-N-(pyrazolo[1,5-a]pyrimidin-3-yl)imidazo[1,2-a]pyrimidine-6-carboxamide as a yellow solid (7.6 mg, 36%). LCMS m/z=434.2 [M+H]⁺, ¹H NMR (400 MHz, MeOH-d₄) δ: 9.41 (s, 1H), 8.86 (dd, 1H), 8.80 (s, 1H), 8.54 (dd, 1H), 7.65 (s, 1H), 7.03 (dd, 1H), 5.84-5.63 (m, 1H), 4.52 (s, 1H), 3.96 (dd, 1H), 3.87 (d, 1H), 2.23-1.83 (m, 6H), 1.68 (d, 6H).

### Example 566: 7-Isopropoxy-N-(1-methyl-1H-pyrazol-3-yl)-2-(4-methyl-2-oxabicyclo[2.2.2]octan-1-yl)imidazo[1,2-a]pyrimidine-6-carboxamide

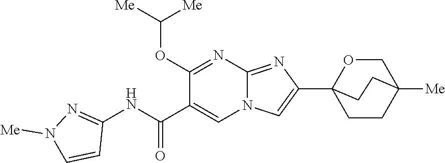 [SMILES: CC(C)Oc1nc2nc(C34CCC(C)(CC3)CO4)cn2cc1C(=O)Nc1ccn(C)n1]

T3P® (50 wt. % in EtOAc, 903 mg, 1.42 mmol) was added to a mixture of 7-isopropoxy-2-(4-methyl-2-oxabicyclo[2.2.2]octan-1-yl)imidazo[1,2-a]pyrimidine-6-carboxylic acid (Preparation 312, 98 mg, 0.284 mmol) and 1-methyl-1H-pyrazol-3-amine (38.6 mg, 0.397 mmol) in pyridine (1 mL) and the reaction stirred 2 h at rt. The mixture was diluted with brine and extracted with EtOAc (3×), dried (MgSO₄) and evaporated to dryness in vacuo. The residue was purified by silica gel chromatography (12 g, 0-50% 3:1 EtOAc/EtOH in heptanes) to obtain 7-isopropoxy-N-(1-methyl-1H-pyrazol-3-yl)-2-(4-methyl-2-oxabicyclo[2.2.2]octan-1-yl)imidazo[1,2-a]pyrimidine-6-carboxamide (39.9 mg, 33%), LCMS m/z=425.3 [M+H]⁺, ¹H NMR (400 MHz, CDCl₃) δ: 1.10 (s, 3H), 1.49 (d, 6H), 1.61-1.73 (m, 2H), 1.85-1.97 (m, 4H), 2.09-2.18 (m, 2H), 3.77 (s, 3H), 4.04 (s, 2H), 5.67-5.78 (m, 1H), 6.67 (d, 1H), 7.08 (s, 1H), 7.22 (d, 1H), 9.10 (s, 1H), 10.01 (s, 1H).

### Example 567: 7-Isopropoxy-2-(4-methyl-2-oxabicyclo[2.2.2]octan-1-yl)-N-(6-methylpyrazolo[1,5-a]pyrimidin-3-yl)imidazo[1,2-a]pyrimidine-6-carboxamide

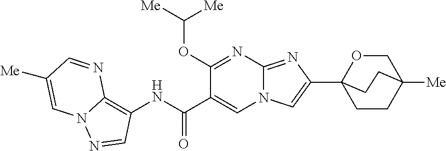 [SMILES: Cc1cnc2c(NC(=O)c3cn4cc(C56CCC(C)(CC5)CO6)nc4nc3OC(C)C)cnn2c1]

T3P® (50 wt. % in EtOAc, 903 mg, 1.42 mmol) was added to a mixture of 7-isopropoxy-2-(4-methyl-2-oxabicyclo[2.2.2]octan-1-yl)imidazo[1,2-a]pyrimidine-6-carboxylic acid (Preparation 312, 98 mg, 0.284 mmol) and 6-methylpyrazolo[1,5-a]pyrimidin-3-amine (58.9 mg, 0.397 mmol) in pyridine (1 mL) and the reaction stirred 2 h at rt. The mixture was diluted with brine and extracted with EtOAc (3×), dried (MgSO₄) and evaporated to dryness in vacuo. The residue was purified by silica gel chromatography (12 g, 0-50% 3:1 EtOAc/EtOH in heptanes) to obtain 7-isopropoxy-2-(4-methyl-2-oxabicyclo[2.2.2]octan-1-yl)-N-(6-methylpyrazolo[1,5-a]pyrimidin-3-yl)imidazo[1,2-a]pyrimidine-6-carboxamide (31 mg, 20.7%). LCMS m/z=476.3 [M+H]⁺, ¹H NMR (400 MHz, CDCl₃) δ: 1.10 (s, 3H), 1.56 (d, 6H), 1.64-1.73 (m, 2H), 1.88-1.97 (m, 4H), 2.10-2.21 (m, 2H), 2.32 (s, 3H), 4.04-4.08 (m, 2H), 5.77 (spt, 1H), 7.11 (s, 1H), 8.24 (d, 1H), 8.33 (s, 1H), 8.75 (s, 1H), 9.14 (s, 1H), 10.34 (s, 1H).

### Example 568: 7-(Cyclopentyloxy)-2-(1-methyl-2-oxabicyclo[2.1.1]hexan-4-yl)-N-(6-methylpyrazolo[1,5-a]pyrimidin-3-yl)imidazo[1,2-a]pyridine-6-carboxamide

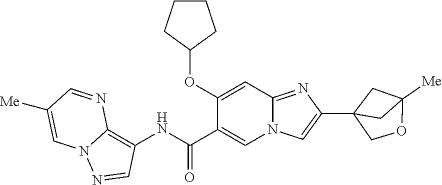 [SMILES: Cc1cnc2c(NC(=O)c3cn4cc(C56COC(C)(C5)C6)nc4cc3OC3CCCC3)cnn2c1]

DIPEA (37.75 mg, 0.292 mmol) and HATU (58.5 mg, 0.153 mmol) were added to 7-(cyclopentyloxy)-2-(1-methyl-2-oxabicyclo[2.1.1]hexan-4-yl)imidazo[1,2-a]pyridine-6-carboxylic acid (Preparation 310, 50 mg, 0.146 mmol) in DMF (5 mL). To this was added 6-methylpyrazolo[1,5-a]pyrimidin-3-amine (26.0 mg, 0.175 mmol) and the reaction stirred at rt overnight. The reaction was evaporated to dryness and the residue purified by prep-HPLC-F (gradient, 10-70%) to afford 7-(cyclopentyloxy)-2-(1-methyl-2-oxabicyclo[2.1.1]hexan-4-yl)-N-(6-methylpyrazolo[1,5-a]pyrimidin-3-yl)imidazo[1,2-a]pyridine-6-carboxamide (23 mg, 33%). LCMS m/z=473 [M+H]⁺; ¹H NMR (500 MHz, DMSO-d₆) δ: 10.38 (s, 1H), 9.19 (s, 1H), 8.94 (d, 1H), 8.66 (s, 1H), 8.46 (d, 1H), 7.84 (s, 1H), 7.13 (s, 1H), 5.21 (br s, 1H), 3.89 (s, 2H), 2.35 (s, 3H), 2.07-2.19 (m, 4H), 2.00 (dd, 2H), 1.82-1.95 (m, 2H), 1.76 (dd, 2H), 1.61-1.74 (m, 1H), 1.68 (br s, 1H), 1.43 (s, 3H).

### Example 569: 7-Isopropoxy-2-(1-methyl-2-oxabicyclo[2.2.1]heptan-4-yl)-N-(6-methylpyrazolo[1,5-a]pyrimidin-3-yl)imidazo[1,2-a]pyrimidine-6-carboxamide

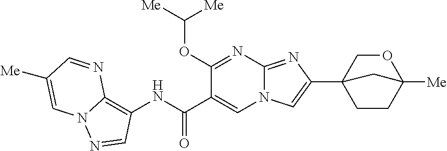 [SMILES: Cc1cnc2c(NC(=O)c3cn4cc(C56CCC(C)(C5)OC6)nc4nc3OC(C)C)cnn2c1]

To a mixture of 7-isopropoxy-2-(1-methyl-2-oxabicyclo[2.2.1]heptan-4-yl)imidazo[1,2-a]pyrimidine-6-carboxylic acid (Preparation 345, 44.8 mg, 0.100 mmol, 2NaCl) and 6-methylpyrazolo[1,5-a]pyrimidin-3-amine (17.8 mg, 0.120 mmol) in DMF (1 mL) was added HATU (40.0 mg, 0.105 mmol) and DIPEA (38.78 mg, 0.300 mmol) and the mixture stirred at rt for 2.5 h. The reaction mixture was diluted with H₂O and MeCN/EtOAc. The resulting solid was collected by filtration and washed with water, EtOAc/MeCN to afford the title compound as a yellow solid (16 mg). The filtrate was separated and extracted with EtOAc. The combined organics were evaporated to dryness and the residue purified by column chromatography (SiO₂, EtOAc/EtOH; 7/1) to give a solid which was combined with the solid above to give 7-isopropoxy-2-(1-methyl-2-oxabicyclo[2.2.1]heptan-4-yl)-N-(6-methylpyrazolo[1,5-a]pyrimidin-3-yl)imidazo[1,2-a]pyrimidine-6-carboxamide as a yellow solid (50 mg). LCMS m/z=462.2 [M+H]⁺; ¹H NMR (400 MHz, MeOH-d₄) δ: 9.40 (s, 1H), 8.71 (s, 1H), 8.68 (dd, 1H), 8.47 (d, 1H), 7.63 (s, 1H), 5.75 (quin, 1H), 4.07 (dd, 1H), 3.94 (d, 1H), 2.43 (d, 3H), 1.76-2.24 (m, 6H), 1.67 (d, 6H), 1.48 (s, 3H).

### Example 570: 7-Cyclobutoxy-N-(1-methyl-1H-pyrazol-3-yl)-2-(1-methyl-2-oxabicyclo[2.2.2]octan-4-yl)imidazo[1,2-a]pyridine-6-carboxamide

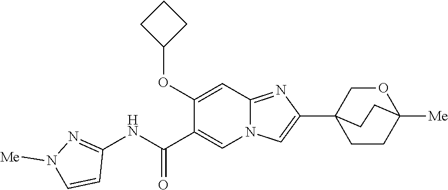 [SMILES: Cn1ccc(NC(=O)c2cn3cc(C45CCC(C)(CC4)OC5)nc3cc2OC2CCC2)n1]

HATU (54.9 mg, 0.144 mmol) and DIPEA (53.1 mg, 0.412 mmol) were to a solution of 7-cyclobutoxy-2-(1-methyl-2-oxabicyclo[2.2.2]octan-4-yl)imidazo[1,2-a]pyridine-6-carboxylic acid (Preparation 297, 67 mg, 0.137 mmol) and 1-methyl-1H-pyrazol-3-amine (22 mg, 0.226 mmol) in DMF (1 mL) and the mixture stirred at rt for 3 h. The mixture was evaporated to dryness in vacuo and the residue partitioned between EtOAc and H₂O. The combined organics were evaporated to dryness and the residue purified by column chromatography (SiO₂, EtOAc/EtOH, 1/0 to 7/1) to afford 7-cyclobutoxy-N-(1-methyl-1H-pyrazol-3-yl)-2-(1-methyl-2-oxabicyclo[2.2.2]octan-4-yl)imidazo[1,2-a]pyridine-6-carboxamide as a white solid (51 mg, 85%). LCMS m/z=436.2 [M+H]⁺; ¹H NMR (400 MHz, MeOH-d₄) δ: 1.16 (s, 3H), 1.74-2.09 (m, 8H), 2.10-2.27 (m, 2H), 2.29-2.49 (m, 2H), 2.57-2.75 (m, 2H), 3.86 (s, 3H), 4.00-4.11 (m, 2H), 4.94-5.08 (m, 1H), 6.69 (d, 1H), 6.79 (s, 1H), 7.54 (d, 1H), 7.60 (s, 1H), 9.02 (s, 1H).

### Example 571: 7-Isopropoxy-N-(1-methyl-1H-pyrazol-3-yl)-2-(1-methyl-2-oxabicyclo[2.2.2]octan-4-yl)imidazo[1,2-a]pyridine-6-carboxamide

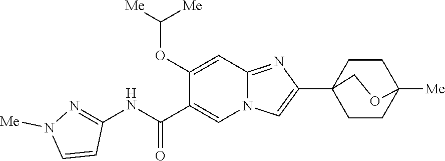 [SMILES: CC(C)Oc1cc2nc(C34CCC(C)(CC3)OC4)cn2cc1C(=O)Nc1ccn(C)n1]

T3P® (50 wt. % in EtOAc, 923 mg, 1.45 mmol) was added to a mixture of 7-isopropoxy-2-(1-methyl-2-oxabicyclo[2.2.2]octan-4-yl)imidazo[1,2-a]pyridine-6-carboxylic acid (Preparation 309, 100 mg, 0.290 mmol) and 1-methyl-1H-pyrazol-3-amine (33.8 mg, 0.348 mmol) in pyridine (1 mL) and the reaction stirred 2 h at rt. The mixture was diluted with brine and extracted with EtOAc (3×), dried (MgSO₄) and evaporated to dryness in vacuo. The residue was purified by silica gel chromatography (12 g, 0-50% 3:1 EtOAc/EtOH in heptanes) to obtain 7-isopropoxy-N-(1-methyl-1H-pyrazol-3-yl)-2-(1-methyl-2-oxabicyclo[2.2.2]octan-4-yl)imidazo[1,2-a]pyridine-6-carboxamide (54.7 mg, 44%). LCMS m/z=424.3 [M+H]⁺, ¹H NMR (400 MHz, CDCl₃) δ: 1.19 (s, 3H), 1.56-1.62 (m, 6H), 1.70-1.83 (m, 2H), 1.93-2.04 (m, 4H), 2.15-2.25 (m, 2H), 3.85 (s, 3H), 4.13 (s, 2H), 4.79 (dt, 1H), 6.77 (d, 1H), 6.96 (s, 1H), 7.28-7.32 (m, 2H), 9.01 (s, 1H), 10.26 (s, 1H).

### Example 572: 7-Isopropoxy-2-(1-methyl-2-oxabicyclo[2.2.2]octan-4-yl)-N-(pyrazolo[1,5-a]pyrimidin-3-yl)imidazo[1,2-a]pyridine-6-carboxamide

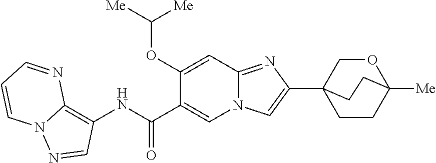 [SMILES: CC(C)Oc1cc2nc(C34CCC(C)(CC3)OC4)cn2cc1C(=O)Nc1cnn2cccnc12]

T3P® (50 wt. % in EtOAc, 923 mg, 1.45 mmol) was added to a mixture of 7-isopropoxy-2-(1-methyl-2-oxabicyclo[2.2.2]octan-4-yl)imidazo[1,2-a]pyridine-6-carboxylic acid (Preparation 309, 100 mg, 0.290 mmol) and pyrazolo[1,5-a]pyrimidin-3-amine (46.7 mg, 0.348 mmol) in pyridine (1 mL) and the reaction stirred 2 h at rt. The mixture was diluted with brine and extracted with EtOAc (3×), dried (MgSO₄) and evaporated to dryness in vacuo. The residue was purified by silica gel chromatography (12 g, 0-50% 3:1 EtOAc/EtOH in heptanes) to obtain 7-isopropoxy-2-(1-methyl-2-oxabicyclo[2.2.2]octan-4-yl)-N-(pyrazolo[1,5-a]pyrimidin-3-yl)imidazo[1,2-a]pyridine-6-carboxamide (30 mg, 22%). LCMS m/z=461.2 [M+H]⁺, ¹H NMR (400 MHz, CDCl₃) δ: 1.20 (s, 3H), 1.67 (d, 6H), 1.74-1.89 (m, 2H), 1.93-2.10 (m, 4H), 2.18-2.28 (m, 2H), 4.13 (s, 2H), 4.82-4.94 (m, 1H), 6.83 (dd, 1H), 7.01 (s, 1H), 7.30 (s, 1H), 8.41 (dd, 1H), 8.63 (dd, 1H), 8.97 (s, 1H), 9.08 (s, 1H), 10.58 (s, 1H).

### Example 573: 7-Isopropoxy-2-(1-methyl-2-oxabicyclo[2.2.2]octan-4-yl)-N-(6-methylpyrazolo[1,5-a]pyrimidin-3-yl)imidazo[1,2-a]pyridine-6-carboxamide

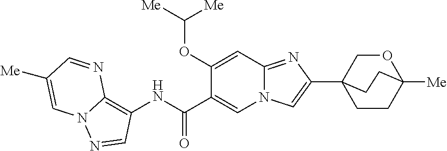 [SMILES: Cc1cnc2c(NC(=O)c3cn4cc(C56CCC(C)(CC5)OC6)nc4cc3OC(C)C)cnn2c1]

T3P® (50 wt. % in EtOAc, 943 mg, 1.52 mmol) was added to a mixture of 7-isopropoxy-2-(1-methyl-2-oxabicyclo[2.2.2]octan-4-yl)imidazo[1,2-a]pyridine-6-carboxylic acid (Preparation 309, 100 mg, 0.290 mmol) and 6-methylpyrazolo[1,5-a]pyrimidin-3-amine (51.6 mg, 0.348 mmol) in pyridine (1 mL) and the reaction stirred 2 h at rt. The mixture was diluted with brine and extracted with EtOAc (3×), dried (MgSO₄) and evaporated to dryness in vacuo. The residue was purified by silica gel chromatography (12 g, 0-50% 3:1 EtOAc/EtOH in heptanes) to obtain 7-isopropoxy-2-(1-methyl-2-oxabicyclo[2.2.2]octan-4-yl)-N-(6-methylpyrazolo[1,5-a]pyrimidin-3-yl)imidazo[1,2-a]pyridine-6-carboxamide (26.8 mg, 19%). LCMS m/z=475.3 [M+H]⁺, ¹H NMR (400 MHz, CDCl₃) δ: 1.11 (s, 3H), 1.60 (d, 6H), 1.67-1.78 (m, 2H), 1.88-2.05 (m, 4H), 2.13 (br d, 2H), 3.40 (s, 3H), 3.65 (quint, 1H), 4.05 (s, 2H), 4.90 (s, 1H), 7.18 (s, 1H), 8.23 (d, 1H), 8.33 (d, 1H), 8.76 (s, 1H), 9.03 (s, 1H), 10.41 (br s, 1H).

### Example 574: 7-Isopropoxy-N-(1-methyl-2-oxo-1,2-dihydropyridin-3-yl)-2-(1-methyl-2-oxabicyclo[2.2.2]octan-4-yl)imidazo[1,2-a]pyridine-6-carboxamide

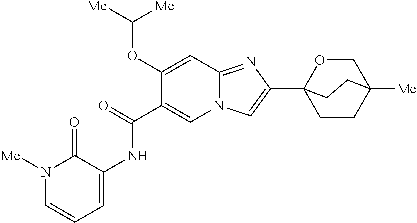 [SMILES: CC(C)Oc1cc2nc(C34CCC(C)(CC3)CO4)cn2cc1C(=O)Nc1cccn(C)c1=O]

T3P® (50 wt. % in EtOAc, 923 mg, 1.45 mmol) was added to a mixture of 7-isopropoxy-2-(1-methyl-2-oxabicyclo[2.2.2]octan-4-yl)imidazo[1,2-a]pyridine-6-carboxylic acid (Preparation 309, 100 mg, 0.290 mmol) and 3-amino-1-methylpyridin-2-one (43.2 mg, 0.348 mmol) in pyridine (1 mL) and the reaction stirred 2 h at rt. The mixture was diluted with brine and extracted with EtOAc (3×), dried (MgSO₄) and evaporated to dryness in vacuo. The residue was purified by silica gel chromatography (12 g, 0-50% 3:1 EtOAc/EtOH in heptanes) to obtain 7-isopropoxy-N-(1-methyl-2-oxo-1,2-dihydropyridin-3-yl)-2-(1-methyl-2-oxabicyclo[2.2.2]octan-4-yl)imidazo[1,2-a]pyridine-6-carboxamide (94 mg, 72%). LCMS m/z=451.2 [M+H]⁺, ¹H NMR (400 MHz, CDCl₃) δ: 1.18 (s, 3H), 1.55-1.65 (m, 6H), 1.70-1.84 (m, 2H), 1.90-2.02 (m, 4H), 2.15-2.29 (m, 2H), 3.64 (s, 3H), 4.09-4.13 (m, 2H), 4.81 (dt, 1H), 6.21-6.29 (m, 1H), 6.96 (s, 1H), 7.03 (dt, 1H), 7.22-7.26 (m, 1H), 8.58 (dt, 1H), 8.92-9.01 (m, 1H), 10.79 (br s, 1H).

### Example 575: 7-Cyclobutoxy-2-(1-methyl-2-oxabicyclo[2.1.1]hexan-4-yl)-N-(6-methylpyrazolo[1,5-a]pyrimidin-3-yl)imidazo[1,2-a]pyrimidine-6-carboxamide

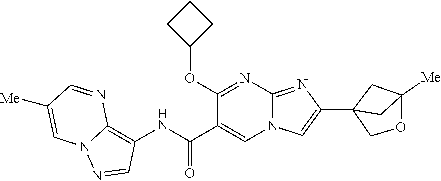 [SMILES: Cc1cnc2c(NC(=O)c3cn4cc(C56COC(C)(C5)C6)nc4nc3OC3CCC3)cnn2c1]

To a solution of 7-cyclobutoxy-2-(1-methyl-2-oxabicyclo[2.1.1]hexan-4-yl)imidazo[1,2-a]pyrimidine-6-carboxylic acid (Preparation 313, 50 mg, 0.152 mmol) and 6-methylpyrazolo[1,5-a]pyrimidin-3-amine (67.5 mg, 0.455 mmol) in T3P® (50 wt. % in EtOAc, 3 mL) was added Pyridine (3 mL) and the mixture stirred at 15° C. for 3 h. The reaction mixture was evaporated to dryness in vacuo and the residue diluted with water (10 mL) and the pH adjusted with aq. NaHCO₃ (10 mL) and extracted with EtOAc (3×20 mL). The combined organics were washed with brine (30 mL), dried (Na₂SO₄) and evaporated to dryness in vacuo. The residue was purified by prep-HPLC-A (gradient, 49-69%) to afford 7-cyclobutoxy-2-(1-methyl-2-oxabicyclo[2.1.1]hexan-4-yl)-N-(6-methylpyrazolo[1,5-a]pyrimidin-3-yl)imidazo[1,2-a]pyrimidine-6-carboxamide as a white solid (10.1 mg, 14%). LCMS m/z=460.2 [M+H]⁺, ¹H NMR (500 MHz, MeOH-d₄) δ: 9.36 (s, 1H), 8.67 (s, 1H), 8.64 (s, 1H), 8.44 (d, 1H), 7.63 (s, 1H), 5.60-5.54 (m, 1H), 4.01 (s, 2H), 2.75-2.65 (m, 2H), 2.62-2.51 (m, 2H), 2.40 (s. 3H), 2.14-2.09 (m, 2H), 2.06-2.00 (m, 1H), 1.91-1.83 (m, 3H), 1.50 (s, 3H).

### Example 576 and 577: N-(6-(difluoromethyl)pyridin-2-yl)-7-isopropoxy-2-((1S,4R)-1-methyl-2-oxabicyclo[2.2.1]heptan-4-yl)imidazo[1,2-a]pyrimidine-6-carboxamide and N-(6-(difluoromethyl)pyridin-2-yl)-7-isopropoxy-2-((1R,4S)-1-methyl-2-oxabicyclo[2.2.1]heptan-4-yl)imidazo[1,2-a]pyrimidine-6-carboxamide

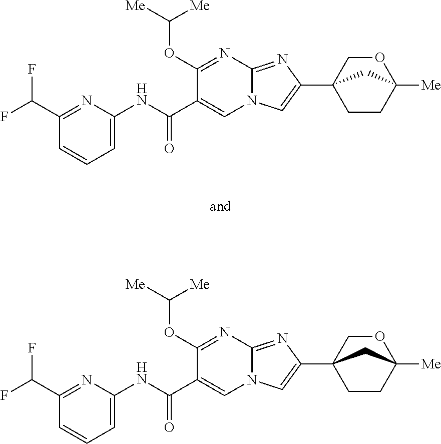 [SMILES: CC(C)Oc1nc2nc([C@@]34CC[C@@](C)(C3)OC4)cn2cc1C(=O)Nc1cccc(C(F)F)n1 ; CC(C)Oc1nc2nc([C@]34CC[C@](C)(C3)OC4)cn2cc1C(=O)Nc1cccc(C(F)F)n1]

*Stereochemistry arbitrarily assigned.

N-(6-(difluoromethyl)pyridin-2-yl)-7-isopropoxy-2-((1S,4R)-1-methyl-2-oxabicyclo[2.2.1]heptan-4-yl)imidazo[1,2-a]pyrimidine-6-carboxamide and N-(6-(difluoromethyl)pyridin-2-yl)-7-isopropoxy-24(1R,4S)-1-methyl-2-oxabicyclo[2.2.1]heptan-4-yl)imidazo[1,2-a]pyrimidine-6-carboxamide were obtained from N-(6-(difluoromethyl)pyridin-2-yl)-7-isopropoxy-2-(1-methyl-2-oxabicyclo[2.2.1]heptan-4-yl)imidazo[1,2-a]pyrimidine-6-carboxamide (55.5 mg, 0.121 mmol, Example 247) by chiral SFC chromatography (LUX Cellulose-4 LC 30×250 mm, 5 μm; 50% MeOH in CO₂).

*Peak 1: Example 576: Yield 19 mg, 34%; LCMS m/z=458.2 [M+H]⁺; ¹H NMR (400 MHz, CDCl₃) δ: 10.54 (s, 1H), 9.19 (s, 1H), 8.44 (d, 1H), 7.90 (t, 1H), 7.42 (d, 1H), 6.25-6.74 (m, 1H), 5.79 (td, 1H), 4.12 (dd, 1H), 3.96 (d, 1H), 1.73-2.31 (m, 6H), 1.60 (d, 6H), 1.48 (s, 3H).

*Peak 2: Example 577: Yield 15 mg, 27%; LCMS m/z=458.2 [M+H]⁺; ¹H NMR (400 MHz, CDCl₃) δ: 10.54 (s, 1H), 9.19 (s, 1H), 8.44 (d, 1H), 7.81-7.96 (m, 1H), 7.42 (d, 1H), 6.28-6.71 (m, 1H), 5.70-5.85 (m, 1H), 4.12 (dd, 1H), 3.96 (d, 1H), 1.77-2.26 (m, 6H), 1.60 (d, 6H), 1.48 (s, 3H).

### Example 578 and Example 579: 7-Isopropoxy-N-(2-methoxypyridin-3-yl)-2-((1S,4R)-1-methyl-2-oxabicyclo[2.2.1]heptan-4-yl)imidazo[1,2-a]pyrimidine-6-carboxamide and 7-isopropoxy-N-(2-methoxypyridin-3-yl)-2-((1R,4S)-1-methyl-2-oxabicyclo[2.2.1]heptan-4-yl)imidazo[1,2-a]pyrimidine-6-carboxamide

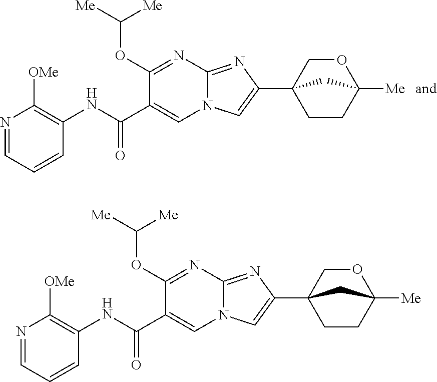 [SMILES: COc1ncccc1NC(=O)c1cn2cc([C@@]34CC[C@@](C)(C3)OC4)nc2nc1OC(C)C ; COc1ncccc1NC(=O)c1cn2cc([C@]34CC[C@](C)(C3)OC4)nc2nc1OC(C)C]

*Stereochemistry arbitrarily assigned.

7-Isopropoxy-N-(2-methoxypyridin-3-yl)-2-((1S,4R)-1-methyl-2-oxabicyclo[2.2.1]heptan-4-yl)imidazo[1,2-a]pyrimidine-6-carboxamide and 7-isopropoxy-N-(2-methoxypyridin-3-yl)-2-((1R,4S)-1-methyl-2-oxabicyclo[2.2.1]heptan-4-yl)imidazo[1,2-a]pyrimidine-6-carboxamide were obtained from 7-isopropoxy-N-(2-methoxypyridin-3-yl)-2-(1-methyl-2-oxabicyclo[2.2.1]heptan-4-yl)imidazo[1,2-a]pyrimidine-6-carboxamide (Example 248, 57 mg, 0.130 mmol) by chiral SFC chromatography (CHIRALPAK AD-H 30×250 mm 5 μm; 50% MeOH in CO₂).

*Peak 1: Example 578): Yield 20 mg, 35%; LCMS m/z=438.2 [M+H]⁺; ¹H NMR (400 MHz, CDCl₃) δ: 10.25 (s, 1H), 9.18 (s, 1H), 8.80 (dd, 1H), 7.92 (dd, 1H), 7.24 (s, 1H), 6.92-7.04 (m, 1H), 5.89 (spt, 1H), 4.10-4.15 (m, 1H), 4.10 (s, 3H), 3.96 (d, 1H), 1.74-2.28 (m, 6H), 1.57 (d, 6H), 1.48 (s, 3H).

*Peak 2: Example 579: Yield 20 mg, 35%; LCMS m/z=438.2 [M+H]⁺; ¹H NMR (400 MHz, CDCl₃) δ: 10.25 (s, 1H), 9.18 (s, 1H), 8.80 (dd, 1H), 7.92 (dd, 1H), 7.24 (s, 1H), 6.96 (dd, 1H), 5.89 (spt, 1H), 4.12 (dd, 1H), 4.10 (s, 3H), 3.96 (d, 1H), 1.73-2.26 (m, 6H), 1.57 (d, 6H), 1.48 (s, 3H).

### Example 580 and 581: 2-((1S,4R)-2-oxabicyclo[2.2.1]heptan-4-yl)-7-isopropoxy-N-(1-methyl-1H-pyrazol-3-yl)imidazo[1,2-a]pyridine-6-carboxamide and 2-((1R,4S)-2-oxabicyclo[2.2.1]heptan-4-yl)-7-isopropoxy-N-(1-methyl-1H-pyrazol-3-yl)imidazo[1,2-a]pyridine-6-carboxamide

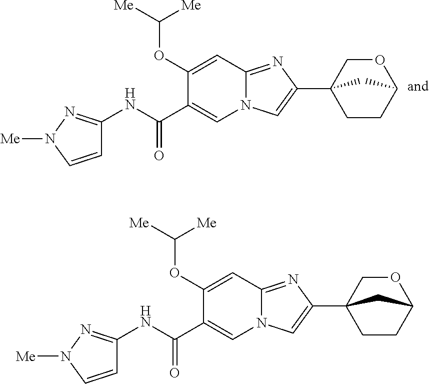 [SMILES: CC(C)Oc1cc2nc([C@@]34CC[C@@H](C3)OC4)cn2cc1C(=O)Nc1ccn(C)n1 ; CC(C)Oc1cc2nc([C@]34CC[C@H](C3)OC4)cn2cc1C(=O)Nc1ccn(C)n1]

*Stereochemistry arbitrarily assigned.

2-((1S,4R)-2-oxabicyclo[2.2.1]heptan-4-yl)-7-isopropoxy-N-(1-methyl-1H-pyrazol-3-yl)imidazo[1,2-a]pyridine-6-carboxamide and 2-((1R,4S)-2-oxabicyclo[2.2.1]heptan-4-yl)-7-isopropoxy-N-(1-methyl-1H-pyrazol-3-yl)imidazo[1,2-a]pyridine-6-carboxamide were obtained from 2-(2-oxabicyclo[2.2.1]heptan-4-yl)-7-isopropoxy-N-(1-methyl-1H-pyrazol-3-yl)imidazo[1,2-a]pyridine-6-carboxamide (Example 415, 58.9 mg, 0.148 mmol) by chiral SFC chromatography (CHIRALPAK IB 30×250 mm, 5 μm; 50% MeOH CO₂).

*Peak 1: Example 580: Yield 22 mg, 37%; LCMS m/z=396.2 [M+H]⁺; ¹H NMR (400 MHz, CDCl₃) δ: 10.24 (s, 1H), 9.02 (s, 1H), 7.34 (s, 1H), 7.29 (d, 1H), 6.76 (d, 1H), 4.72-4.92 (m, 1H), 4.52 (s, 1H), 3.99 (dd, 1H), 3.90 (d, 1H), 3.84 (s, 3H), 1.77-2.21 (m, 6H), 1.58 (d, 6H).

*Peak 2: Example 581: Yield 22 mg, 37%; LCMS m/z=396.2 [M+H]⁺; ¹H NMR (400 MHz, CDCl₃) δ: 10.24 (s, 1H), 9.02 (s, 1H), 7.34 (s, 1H), 7.29 (d, 1H), 6.76 (d, 1H), 4.73-4.88 (m, 1H), 4.52 (s, 1H), 3.99 (dd, 1H), 3.90 (d, 1H), 3.84 (s, 3H), 1.81-2.22 (m, 6H), 1.58 (d, 6H).

### Example 582 and Example 583: 2-((1S,4R)-2-oxabicyclo[2.2.1]heptan-4-yl)-7-isopropoxy-N-(pyrazolo[1,5-a]pyrimidin-3-yl)imidazo[1,2-a]pyridine-6-carboxamide and 2-((1R,4S)-2-oxabicyclo[2.2.1]heptan-4-yl)-7-isopropoxy-N-(pyrazolo[1,5-a]pyrimidin-3-yl)imidazo[1,2-a]pyridine-6-carboxamide

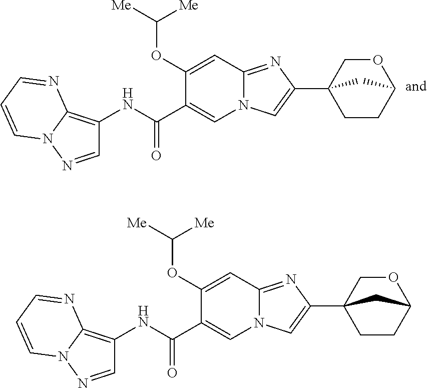 [SMILES: CC(C)Oc1cc2nc([C@@]34CC[C@@H](C3)OC4)cn2cc1C(=O)Nc1cnn2cccnc12 ; CC(C)Oc1cc2nc([C@]34CC[C@H](C3)OC4)cn2cc1C(=O)Nc1cnn2cccnc12]

*Stereochemistry arbitrarily assigned.

2-((1S,4R)-2-oxabicyclo[2.2.1]heptan-4-yl)-7-isopropoxy-N-(pyrazolo[1,5-a]pyrimidin-3-yl)imidazo[1,2-a]pyridine-6-carboxamide and 2-((1R,4S)-2-oxabicyclo[2.2.1]heptan-4-yl)-7-isopropoxy-N-(pyrazolo[1,5-a]pyrimidin-3-yl)imidazo[1,2-a]pyridine-6-carboxamide were obtained from 2-(2-oxabicyclo[2.2.1]heptan-4-yl)-7-isopropoxy-N-(pyrazolo[1,5-a]pyrimidin-3-yl)imidazo[1,2-a]pyridine-6-carboxamide (Example 416, 58.9 mg, 0.148 mmol) by chiral SFC chromatography (CHIRALPAK IB 30×250 mm 5 μm; 50% MeOH in CO₂).

*Peak 1: Example 582: Yield 17.3 mg, 28%; LCMS m/z=433.2 [M+H]⁺; ¹H NMR (400 MHz, CDCl₃) δ: 10.57 (s, 1H), 9.08 (s, 1H), 8.95 (s, 1H), 8.61 (dd, 1H), 8.40 (dd, 1H), 7.38 (s, 1H), 6.98-7.10 (m, 1H), 6.81 (dd, 1H), 4.87 (td, 1H), 4.52 (s, 1H), 4.01 (dd, 1H), 3.91 (d, 1H), 1.79-2.26 (m, 6H), 1.66 (d, 6H).

*Peak 2: Example 583: Yield 17.2 mg, 28%; LCMS m/z=433.2 [M+H]⁺; ¹H NMR (400 MHz, CDCl₃) δ: 10.57 (s, 1H), 9.08 (s, 1H), 8.95 (s, 1H), 8.61 (dd, 1H), 8.40 (dd, 1H), 7.38 (s, 1H), 6.96-7.05 (m, 1H), 6.81 (dd, 1H), 4.76-4.95 (m, 1H), 4.52 (s, 1H), 4.01 (dd, 1H), 3.91 (d, 1H), 1.77-2.23 (m, 6H), 1.66 (d, 6H).

### Example 584 and 585: 8-(Difluoromethoxy)-N-(6-(difluoromethyl)pyridin-2-yl)-2-((1S,4R)-1-methyl-2-oxabicyclo[2.2.1]heptan-4-yl)imidazo[1,2-a]pyridine-6-carboxamide and 8-(difluoromethoxy)-N-(6-(difluoromethyl)pyridin-2-yl)-2-((1R,4S)-1-methyl-2-oxabicyclo[2.2.1]heptan-4-yl)imidazo[1,2-a]pyridine-6-carboxamide

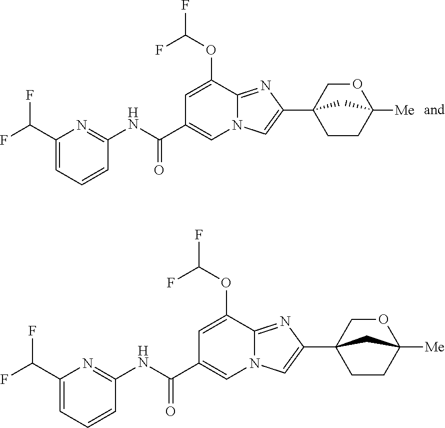 [SMILES: C[C@@]12CC[C@@](c3cn4cc(C(=O)Nc5cccc(C(F)F)n5)cc(OC(F)F)c4n3)(CO1)C2 ; C[C@]12CC[C@](c3cn4cc(C(=O)Nc5cccc(C(F)F)n5)cc(OC(F)F)c4n3)(CO1)C2]

*Stereochemistry arbitrarily assigned.

8-(Difluoromethoxy)-N-(6-(difluoromethyl)pyridin-2-yl)-2-((1S,4R)-1-methyl-2-oxabicyclo[2.2.1]heptan-4-yl)imidazo[1,2-a]pyridine-6-carboxamide and 8-(difluoromethoxy)-N-(6-(difluoromethyl)pyridin-2-yl)-2-((1R,4S)-1-methyl-2-oxabicyclo[2.2.1]heptan-4-yl)imidazo[1,2-a]pyridine-6-carboxamide were obtained from 8-(difluoromethoxy)-N-(6-(difluoromethyl)pyridin-2-yl)-2-(1-methyl-2-oxabicyclo[2.2.1]heptan-4-yl)imidazo[1,2-a]pyridine-6-carboxamide (Example 251, 90 mg, 0.208 mmol) by chiral SFC chromatography (CHIRALPAK AH-H 30×250 mm 5 μm; 40% MeOH in CO₂).

*Peak 1: Example 584: Yield 28.4 mg, 29%; LCMS m/z=465.2 [M+H]⁺;

*Peak 2: Example 585: Yield 29.1 mg, 30%; LCMS m/z=465.2 [M+H]⁺; ¹H NMR (500 MHz, DMSO-d₆) δ 11.24 (s, 1H), 9.21 (d, J=1.37 Hz, 1H), 8.32 (d, J=8.24 Hz, 1H), 8.07 (t, J=8.01 Hz, 1H), 8.02 (s, 1H), 7.63 (t, J=73.55 Hz, 1H), 7.55 (s, 1H), 7.49 (d, J=7.48 Hz, 1H), 6.94 (t, J=54.63 Hz, 1H), 3.97 (dd, J=3.36, 6.41 Hz, 1H), 3.81 (d, J=6.41 Hz, 1H), 2.06-2.16 (m, 1H), 1.94-2.02 (m, 1H), 1.77-1.92 (m, 3H), 1.66-1.74 (m, 1H), 1.39 (s, 3H).

### Example 586 and 587: 8-Methoxy-N-(2-methoxypyridin-3-yl)-2-((1R,4S)-1-methyl-2-oxabicyclo[2.2.1]heptan-4-yl)imidazo[1,2-a]pyrazine-6-carboxamide and 8-methoxy-N-(2-methoxypyridin-3-yl)-2-((1S,4R)-1-methyl-2-oxabicyclo[2.2.1]heptan-4-yl)imidazo[1,2-a]pyrazine-6-carboxamide

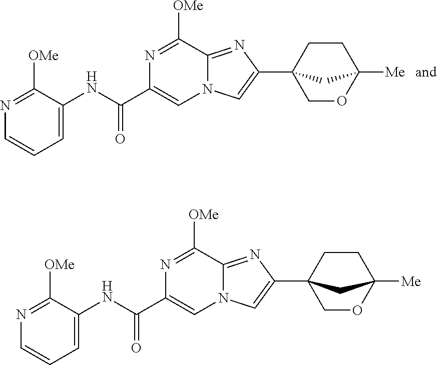 [SMILES: COc1ncccc1NC(=O)c1cn2cc([C@]34CC[C@](C)(C3)OC4)nc2c(OC)n1 ; COc1ncccc1NC(=O)c1cn2cc([C@@]34CC[C@@](C)(C3)OC4)nc2c(OC)n1]

*stereochemistry arbitrarily assigned

T3P® (50 wt. % in EtOAc, 709 mg, 1.11 mmol) and TEA (365 mg, 3.61 mmol) were added to a mixture of 8-methoxy-2-(1-methyl-2-oxabicyclo[2.2.1]heptan-4-yl)imidazo[1,2-a]pyrazine-6-carboxylic acid (Preparation 352, 84 mg, 0.2 mmol, 2NaCl) and 2-methoxypyridin-3-amine (28 mg, 0.226 mmol) and the suspension was heated at 80° C. for 5 min and under mW irradiation at 100° C. for 30 min. The reaction was quenched by the addition of MeOH, EtOAc and H₂O. The aqueous layer was extracted with EtOAc and the combined organics were evaporated to dryness. The residue was triturated with MeCN and the solids washed with H₂O and MeCN to afford 8-methoxy-N-(2-methoxypyridin-3-yl)-2-(1-methyl-2-oxabicyclo[2.2.1]heptan-4-yl)imidazo[1,2-a]pyrazine-6-carboxamide as a white solid (58 mg, 71%) which was purified by preparative chromatography (CHIRALPAK AD-H 30×250 mm 5 μm, 45% MeOH in CO₂) to afford 8-methoxy-N-(2-methoxypyridin-3-yl)-2-((1R,4S)-1-methyl-2-oxabicyclo[2.2.1]heptan-4-yl)imidazo[1,2-a]pyrazine-6-carboxamide and 8-methoxy-N-(2-methoxypyridin-3-yl)-2-((1S,4R)-1-methyl-2-oxabicyclo[2.2.1]heptan-4-yl)imidazo[1,2-a]pyrazine-6-carboxamide.

*Peak 1, Example 586, ¹H NMR (400 MHz, CDCl₃) δ: 1.48 (s, 3H), 1.75-2.26 (m, 6H), 4.00 (d, 1H), 4.09 (s, 3H), 4.13 (dd, 1H), 4.30 (s, 3H), 6.91-7.04 (m, 1H), 7.55 (s, 1H), 7.92 (dd, 1H), 8.64 (s, 1H), 8.76 (dd, 1H), 10.13 (s, 1H).

*Peak 2, Example 587, ¹H NMR (400 MHz, CDCl₃) δ: 1.48 (s, 3H), 1.75-2.26 (m, 6H), 4.00 (d, 1H), 4.09 (s, 3H), 4.13 (dd, 1H), 4.30 (s, 3H), 6.91-7.04 (m, 1H), 7.55 (s, 1H), 7.92 (dd, 1H), 8.64 (s, 1H), 8.76 (dd, 1H), 10.13 (s, 1H).

### Example 588 and 589: N-(6-(difluoromethyl)pyridin-2-yl)-8-isopropoxy-2-((1S,4R)-1-methyl-2-oxabicyclo[2.2.1]heptan-4-yl)imidazo[1,2-a]pyrazine-6-carboxamide and N-(6-(difluoromethyl)pyridin-2-yl)-8-isopropoxy-2-((1R,4S)-1-methyl-2-oxabicyclo[2.2.1]heptan-4-yl)imidazo[1,2-a]pyrazine-6-carboxamide

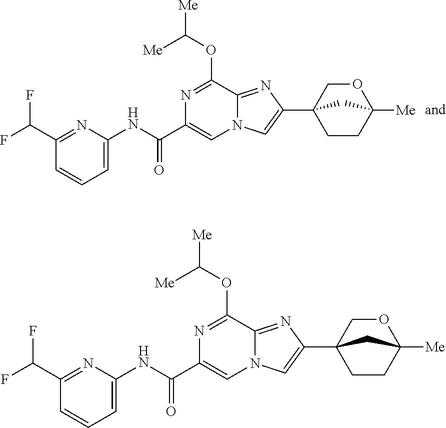 [SMILES: CC(C)Oc1nc(C(=O)Nc2cccc(C(F)F)n2)cn2cc([C@@]34CC[C@@](C)(C3)OC4)nc12 ; CC(C)Oc1nc(C(=O)Nc2cccc(C(F)F)n2)cn2cc([C@]34CC[C@](C)(C3)OC4)nc12]

*stereochemistry arbitrarily assigned

N-(6-(difluoromethyl)pyridin-2-yl)-8-isopropoxy-2-((1S,4R)-1-methyl-2-oxabicyclo[2.2.1]heptan-4-yl)imidazo[1,2-a]pyrazine-6-carboxamide and N-(6-(difluoromethyl)pyridin-2-yl)-8-isopropoxy-2-((1R,4S)-1-methyl-2-oxabicyclo[2.2.1]heptan-4-yl)imidazo[1,2-a]pyrazine-6-carboxamide were prepared from 8-isopropoxy-2-(1-methyl-2-oxabicyclo[2.2.1]heptan-4-yl)imidazo[1,2-a]pyrazine-6-carboxylic acid (Preparation 353) and 6-(difluoromethyl)pyridin-2-amine using an analogous method to that described for Example 586 and 587 using preparative chiral SFC chromatography (CHIRALPAK IB 30×250 mm Sum; 40% MeOH in CO₂).

*Peak 1: Example 588, 17.3 mg, 26%. LCMS m/z=458.2 [M+H]⁺; ¹H NMR (400 MHz, CDCl₃) δ: 9.93 (s, 1H), 8.65 (d, 1H), 8.51 (d, 1H), 7.92 (t, 1H), 7.54 (d, 1H), 7.42 (s, 1H), 6.34-6.77 (m, 1H), 5.64-5.83 (m, 1H), 4.13 (dd, 1H), 4.00 (d, 1H), 1.70-2.29 (m, 6H), 1.61 (dd, 6H), 1.48 (d, 3H).

*Peak 2: Example 589, 18.8 mg, 28%. LCMS m/z=458.2 [M+H]+; 1H NMR (400 MHz, CDCl₃) δ: 9.93 (s, 1H), 8.65 (d, 1H), 8.51 (d, 1H), 7.92 (t, 1H), 7.54 (d, 1H), 7.42 (s, 1H), 6.34-6.77 (m, 1H), 5.64-5.83 (m, 1H), 4.13 (dd, 1H), 4.00 (d, 1H), 1.70-2.29 (m, 6H), 1.61 (dd, 6H), 1.48 (d, 3H).

### Example 590 and Example 591: 2-((1S,4R)-2-oxabicyclo[2.2.1]heptan-4-yl)-N-(6-(difluoromethyl)pyridin-2-yl)-7-isopropoxyimidazo[1,2-a]pyridine-6-carboxamide and 2-((1R,4S)-2-oxabicyclo[2.2.1]heptan-4-yl)-N-(6-(difluoromethyl)pyridin-2-yl)-7-isopropoxyimidazo[1,2-a]pyridine-6-carboxamide

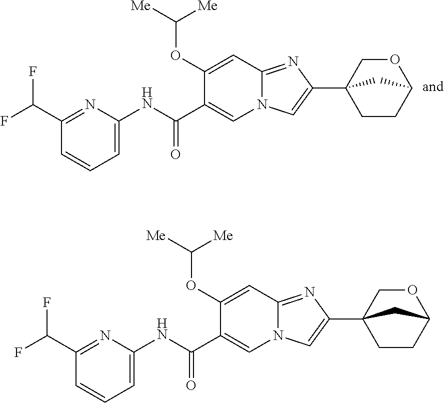 [SMILES: CC(C)Oc1cc2nc([C@@]34CC[C@@H](C3)OC4)cn2cc1C(=O)Nc1cccc(C(F)F)n1 ; CC(C)Oc1cc2nc([C@]34CC[C@H](C3)OC4)cn2cc1C(=O)Nc1cccc(C(F)F)n1]

*stereochemistry arbitrarily assigned

2-((1 S,4R)-2-oxabicyclo[2.2.1]heptan-4-yl)-N-(6-(difluoromethyl)pyridin-2-yl)-7-isopropoxyimidazo[1,2-a]pyridine-6-carboxamide and 2-((1R,4S)-2-oxabicyclo[2.2.1]heptan-4-yl)-N-(6-(difluoromethyl)pyridin-2-yl)-7-isopropoxyimidazo[1,2-a]pyridine-6-carboxamide were prepared from 2-(2-oxabicyclo[2.2.1]heptan-4-yl)-7-isopropoxyimidazo[1,2-a]pyridine-6-carboxylic acid (Preparation 348) and 6-(difluoromethyl)pyridin-2-amine using an analogous method to that described for Example 586 and 587 using preparative chiral SFC chromatography (CHIRALPAK IB 30×250 mm 5 μm; 40% MeOH in CO₂).

*Peak 1, Example 590, 18 mg, 34%. LCMS m/z=443.2 [M+H]+; 1H NMR (400 MHz, CDCl₃) δ: 10.74 (s, 1H), 9.02 (s, 1H), 8.46 (d, 1H), 7.89 (t, 1H), 7.32-7.43 (m, 2H), 6.99 (s, 1H), 6.29-6.69 (m, 1H), 4.74-4.97 (m, 1H), 4.52 (s, 1H), 4.00 (dd, 1H), 3.90 (d, 1H), 1.72-2.23 (m, 6H), 1.61 (d, 6H)

*Peak 2, Example 591, 18 mg, 34%. LCMS m/z=443.2 [M+H]+; 1H NMR (400 MHz, CDCl₃) δ: 10.74 (s, 1H), 9.02 (s, 1H), 8.46 (d, 1H), 7.89 (t, 1H), 7.32-7.45 (m, 2H), 6.99 (s, 1H), 6.27-6.69 (m, 2H), 4.83 (td, 1H), 4.52 (s, 1H), 4.00 (dd, 1H), 3.89 (s, 1H), 1.76-2.18 (m, 6H), 1.61 (d, 6H).

### Example 592 and 593: 8-isopropoxy-N-(1-methyl-1H-pyrazol-3-yl)-2-((1S,4R)-1-methyl-2-oxabicyclo[2.2.1]heptan-4-yl)imidazo[1,2-a]pyrazine-6-carboxamide and 8-isopropoxy-N-(1-methyl-1H-pyrazol-3-yl)-2-((1R,4S)-1-methyl-2-oxabicyclo[2.2.1]heptan-4-yl)imidazo[1,2-a]pyrazine-6-carboxamide

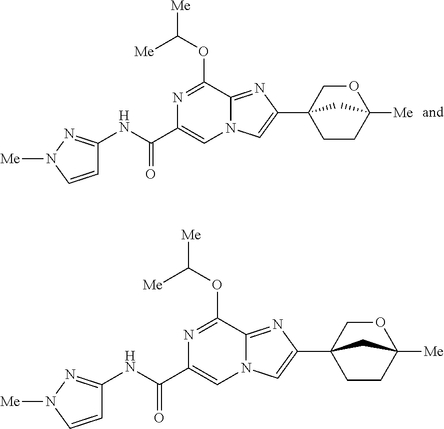 [SMILES: CC(C)Oc1nc(C(=O)Nc2ccn(C)n2)cn2cc([C@@]34CC[C@@](C)(C3)OC4)nc12 ; CC(C)Oc1nc(C(=O)Nc2ccn(C)n2)cn2cc([C@]34CC[C@](C)(C3)OC4)nc12]

*stereochemistry arbitrarily assigned

HATU (125 mg, 0.328 mmol) and DIPEA (155 mg, 1.20 mmol) were added to a mixture of 8-isopropoxy-2-(1-methyl-2-oxabicyclo[2.2.1]heptan-4-yl)imidazo[1,2-a]pyrazine-6-carboxylic acid (Preparation 353, 99.4 mg, 0.222 mmol, 2NaCl) and 1-methylpyrazol-3-amine (42 mg, 0.432 mmol) in DMF (1.5 mL) and stirred at rt for 2.5 h. The reaction was diluted with brine and extracted with EtOAc. The combined organics were evaporated to dryness and the residue purified by column chromatography (24 g, 100% EtOAc). The residue was further purified by preparative chiral SFC chromatography (CHIRALPAK IB 30×250 mm 5 μm; 40% MeOH in CO₂) to afford 8-isopropoxy-N-(1-methyl-1H-pyrazol-3-yl)-2-((1S,4R)-1-methyl-2-oxabicyclo[2.2.1]heptan-4-yl)imidazo[1,2-a]pyrazine-6-carboxamide and 8-isopropoxy-N-(1-methyl-1H-pyrazol-3-yl)-2-((1R,4S)-1-methyl-2-oxabicyclo[2.2.1]heptan-4-yl)imidazo[1,2-a]pyrazine-6-carboxamide.

*Peak 1: Example 592. Peak 1: LCMS m/z=411.2 [M+H]⁺; ¹H NMR (400 MHz, MeOH-d₄) δ: 8.80 (s, 1H), 7.96 (s, 1H), 7.55 (d, 1H), 6.72 (d, 1H), 5.83 (spt, 1H), 3.94-4.11 (m, 2H), 3.87 (s, 3H), 1.75-2.30 (m, 6H), 1.56 (d, 6H), 1.48 (s, 3H).

*Peak 2: Example 593. Peak 2: LCMS m/z=411.2 [M+H]⁺; ¹H NMR (400 MHz, MeOH-d₄) δ: 8.80 (s, 1H), 7.96 (s, 1H), 7.55 (d, 1H), 6.72 (d, 1H), 5.83 (spt, 1H), 3.93-4.17 (m, 2H), 3.87 (s, 3H), 1.76-2.26 (m, 6H), 1.56 (d, 6H), 1.48 (s, 3H).

### Example 594 and 595: (S)-8-ethoxy-N-(5-fluoro-2-methoxypyridin-3-yl)-2-(tetrahydro-2H-pyran-3-yl)imidazo[1,2-a]pyrazine-6-carboxamide and (R)-8-ethoxy-N-(5-fluoro-2-methoxypyridin-3-yl)-2-(tetrahydro-2H-pyran-3-yl)imidazo[1,2-a]pyrazine-6-carboxamide

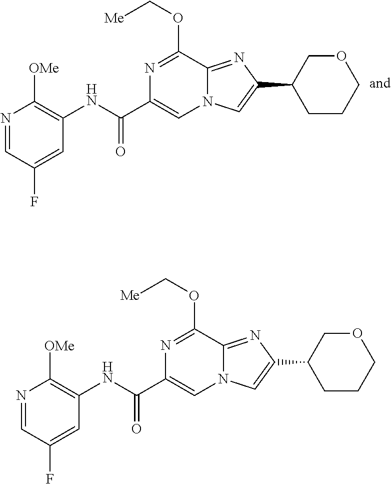 [SMILES: CCOc1nc(C(=O)Nc2cc(F)cnc2OC)cn2cc([C@@H]3CCCOC3)nc12 ; CCOc1nc(C(=O)Nc2cc(F)cnc2OC)cn2cc([C@H]3CCCOC3)nc12]

*stereochemistry arbitrarily assigned

(S)-8-ethoxy-N-(5-fluoro-2-methoxypyridin-3-yl)-2-(tetrahydro-2H-pyran-3-yl)imidazo[1,2-a]pyrazine-6-carboxamide and (R)-8-ethoxy-N-(5-fluoro-2-methoxypyridin-3-yl)-2-(tetrahydro-2H-pyran-3-yl)imidazo[1,2-a]pyrazine-6-carboxamide were obtained from chiral SFC chromatography of Example 271 (CHIRALPAK AD-H; 30×250 mm 5 μm; 40% MeOH+0.1% DEA in CO₂)

*Peak 1, Example 594: LCMS m/z=416.3 [M+H]⁺; ¹H NMR (400 MHz, CDCl₃) δ: 1.66 (t, 3H), 1.74-1.84 (m, 2H), 1.88-2.03 (m, 1H), 2.21 (br dd, 1H), 3.15-3.27 (m, 1H), 3.55-3.65 (m, 1H), 3.69 (dd, 1H), 3.89-3.99 (m, 1H), 4.06-4.11 (m, 3H), 4.19 (dd, 1H), 4.73-4.85 (m, 2H), 7.62 (s, 1H), 7.77 (d, 1H), 8.58-8.73 (m, 2H), 10.17 (s, 1H).

*Peak 2, Example 595: LCMS m/z=416.3 [M+H]⁺; ¹H NMR (400 MHz, CDCl₃) δ: 1.66 (t, 3H), 1.74-1.84 (m, 2H), 1.88-2.03 (m, 1H), 2.21 (br dd, 1H), 3.15-3.27 (m, 1H), 3.55-3.65 (m, 1H), 3.69 (dd, 1H), 3.89-3.99 (m, 1H), 4.06-4.11 (m, 3H), 4.19 (dd, 1H), 4.73-4.85 (m, 2H), 7.62 (s, 1H), 7.77 (d, 1H), 8.58-8.73 (m, 2H), 10.17 (s, 1H).

### Example 596 and Example 597: (S)-8-ethoxy-N-(5-fluoro-1-methyl-2-oxo-1,2-dihydropyridin-3-yl)-2-(tetrahydro-2H-pyran-3-yl)imidazo[1,2-a]pyrazine-6-carboxamide and (R)-8-ethoxy-N-(5-fluoro-1-methyl-2-oxo-1,2-dihydropyridin-3-yl)-2-(tetrahydro-2H-pyran-3-yl)imidazo[1,2-a]pyrazine-6-carboxamide

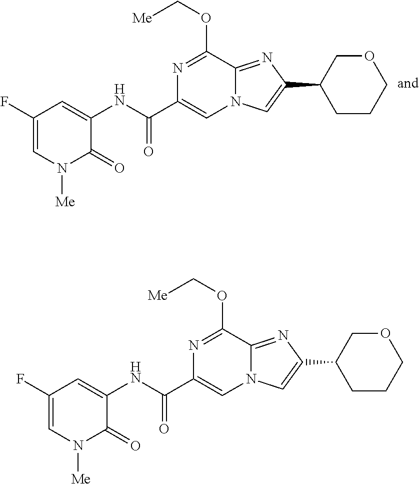 [SMILES: CCOc1nc(C(=O)Nc2cc(F)cn(C)c2=O)cn2cc([C@@H]3CCCOC3)nc12 ; CCOc1nc(C(=O)Nc2cc(F)cn(C)c2=O)cn2cc([C@H]3CCCOC3)nc12]

*stereochemistry arbitrarily assigned

(S)-8-ethoxy-N-(5-fluoro-1-methyl-2-oxo-1,2-dihydropyridin-3-yl)-2-(tetrahydro-2H-pyran-3-yl)imidazo[1,2-a]pyrazine-6-carboxamide and (R)-8-ethoxy-N-(5-fluoro-1-methyl-2-oxo-1,2-dihydropyridin-3-yl)-2-(tetrahydro-2H-pyran-3-yl)imidazo[1,2-a]pyrazine-6-carboxamide were obtained from chiral SFC chromatography of Example 272 (CHIRALPAK IA; 30×250 mm 5 μm; 40% MeOH+0.1% DEA in CO₂)

*Peak 1, Example 596: LCMS m/z=416.3 [M+H]⁺; ¹H NMR (600 MHz, CDCl₃) δ: 1.55 (t, 3H), 1.62-1.71 (m, 2H), 1.82-1.91 (m, 1H), 2.15-2.25 (m, 1H), 3.03-3.15 (m, 1H), 3.43-3.54 (m, 1H), 3.54-3.62 (m, 4H), 3.80-3.92 (m, 1H), 4.05-4.15 (m, 1H), 4.72 (q, 2H), 6.93 (t, 1H), 7.49-7.57 (m, 1H), 8.50 (dd, 1H), 8.54 (s, 1H), 10.52-10.64 (m, 1H).

*Peak 1, Example 597: LCMS m/z=416.3 [M+H]⁺; ¹H NMR (400 MHz, CDCl₃) δ: 1.65 (m, 3H), 1.72-1.77 (m, 2H), 1.94-2.00 (m, 1H), 2.18-2.28 (m, 1H), 3.15-3.26 (m, 1H), 3.55-3.64 (m, 1H), 3.64-3.70 (m, 4H), 3.90-3.99 (m, 1H), 4.14-4.22 (m, 1H), 4.77-4.86 (m, 2H), 7.02 (dd, 1H), 7.60 (s, 1H), 8.56-8.65 (m, 2,H), 10.68 (s, 1H).

### Example 598 and Example 599: (R)-8-ethoxy-N-(2-methoxypyridin-3-yl)-2-(tetrahydrofuran-3-yl)imidazo[1,2-a]pyrazine-6-carboxamide and (S)-8-ethoxy-N-(2-methoxypyridin-3-yl)-2-(tetrahydrofuran-3-yl)imidazo[1,2-a]pyrazine-6-carboxamide

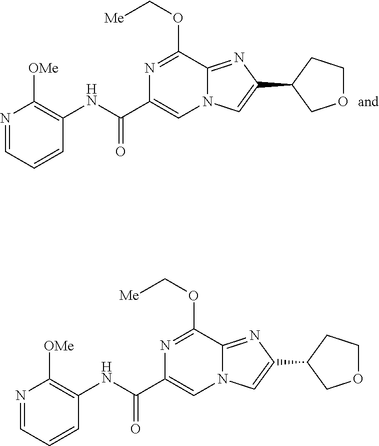 [SMILES: CCOc1nc(C(=O)Nc2cccnc2OC)cn2cc([C@H]3CCOC3)nc12 ; CCOc1nc(C(=O)Nc2cccnc2OC)cn2cc([C@@H]3CCOC3)nc12]

*stereochemistry arbitrarily assigned

(R)-8-ethoxy-N-(2-methoxypyridin-3-yl)-2-(tetrahydrofuran-3-yl)imidazo[1,2-a]pyrazine-6-carboxamide and (S)-8-ethoxy-N-(2-methoxypyridin-3-yl)-2-(tetrahydrofuran-3-yl)imidazo[1,2-a]pyrazine-6-carboxamide were obtained from 8-ethoxy-N-(2-methoxypyridin-3-yl)-2-(tetrahydrofuran-3-yl)imidazo[1,2-a]pyrazine-6-carboxamide (Example 273) by chiral SFC chromatography (CHIRALPAK AD-H 30×250 mm 5 μm; 40% MeOH+0.1% DEA in CO₂).

*Peak 1, Example 598: LCMS m/z=384.3 [M+H]⁺; ¹H NMR (400 MHz, CDCl₃) δ: 10.12 (s, 1H), 8.73 (dd, 1H), 8.55-8.66 (m, 1H), 7.89 (dd, J1H), 7.51-7.61 (m, 1H), 6.95 (dd, 1H), 4.77 (q, 2H), 4.18 (dd, 1H), 3.98-4.10 (m, 4H), 3.89-3.98 (m, 2H), 3.70 (quin, 1H), 2.35-2.50 (m, 1H), 2.13-2.29 (m, 1H), 1.64 (t, 3H).

*Peak 2, Example 599: LCMS m/z=384.2 [M+H]⁺; ¹H NMR (400 MHz, CDCl₃) δ: 10.16 (s, 1H), 8.78 (dd, 1H), 8.63 (s, 1H), 7.94 (dd, 1H), 7.61 (s, 1H), 6.99 (dd, 1H), 4.81 (q, 2H), 4.21 (dd, 1H), 4.11 (s, 3H), 4.03-4.09 (m, 1H), 3.90-4.01 (m, 2H), 3.74 (quin, 1H), 2.40-2.51 (m, 1H), 2.20-2.30 (m, 1H), 1.67 (t, 3H).

### Example 600 and Example 601: N-(6-(difluoromethyl)pyridin-2-yl)-8-ethoxy-2-1R,4S)-1-methyl-2-oxabicyclo[2.2.1]heptan-4-yl)imidazo[1,2-a]pyrazine-6-carboxamide and N-(6-(difluoromethyl)pyridin-2-yl)-8-ethoxy-2-((1S,4R)-1-methyl-2-oxabicyclo[2.2.1]heptan-4-yl)imidazo[1,2-a]pyrazine-6-carboxamide

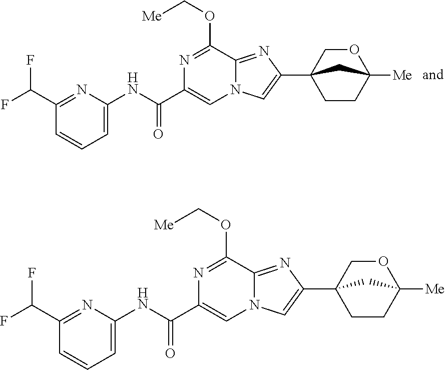 [SMILES: CCOc1nc(C(=O)Nc2cccc(C(F)F)n2)cn2cc([C@]34CC[C@](C)(C3)OC4)nc12 ; CCOc1nc(C(=O)Nc2cccc(C(F)F)n2)cn2cc([C@@]34CC[C@@](C)(C3)OC4)nc12]

*stereochemistry arbitrarily assigned

N-(6-(difluoromethyl)pyridin-2-yl)-8-ethoxy-2-((1R,4S)-1-methyl-2-oxabicyclo[2.2.1]heptan-4-yl)imidazo[1,2-a]pyrazine-6-carboxamide and N-(6-(difluoromethyl)pyridin-2-yl)-8-ethoxy-2-((1S,4R)-1-methyl-2-oxabicyclo[2.2.1]heptan-4-yl)imidazo[1,2-a]pyrazine-6-carboxamide were obtained from N-(6-(difluoromethyl)pyridin-2-yl)-8-ethoxy-2-(1-methyl-2-oxabicyclo[2.2.1]heptan-4-yl)imidazo[1,2-a]pyrazine-6-carboxamide-(Example 274) by chiral SFC chromatography (CHIRALPAK AD-H 30×250 mm 5 μm; 40% MeOH+0.1% DEA in CO₂).

*Peak 1, Example 600: LCMS m/z=444.2 [M+H]⁺; ¹H NMR (400 MHz, CDCl₃) δ: 1.43-1.52 (m, 4H), 1.65 (t, 3H), 1.79-1.91 (m, 3H), 1.98 (d, 1H), 2.02-2.10 (m, 1H), 2.10-2.17 (m, 1H), 2.17-2.27 (m, 1H), 4.02 (d, 1H), 4.14 (dd, 1H), 4.81 (q, 2H), 6.43-6.70 (m, 1H), 7.44 (d, 1H), 7.51-7.62 (m, 1H), 7.93 (t, 1H), 8.52 (d, 1H), 8.64-8.73 (m, 1H), 9.90-10.04 (m, 1H).

*Peak 2, Example 601: LCMS m/z=444.2 [M+H]⁺; ¹H NMR (400 MHz, CDCl₃) δ: 1.50 (s, 3H), 1.65 (t, 3H), 1.79-1.91 (m, 2H), 1.98 (d, 1H), 2.02-2.10 (m, 1H), 2.10-2.17 (m, 1H), 2.17-2.27 (m, 1H), 4.02 (d, 1H), 4.14 (dd, 1H), 4.81 (q, 2H), 6.43-6.70 (m, 1H), 7.44 (d, 1H), 7.51-7.62 (m, 1H), 7.93 (t, 1H), 8.52 (d, 1H), 8.64-8.73 (m, 1H), 9.90-10.04 (m, 1H).

### Example 602 and Example 603: N-(5-fluoro-1-methyl-1H-pyrazol-3-yl)-7-isopropoxy-2-((1R,4S)-1-methyl-2-oxabicyclo[2.2.1]heptan-4-yl)imidazo[1,2-a]pyridine-6-carboxamide and N-(5-fluoro-1-methyl-1H-pyrazol-3-yl)-7-isopropoxy-2-((1S,4R)-1-methyl-2-oxabicyclo[2.2.1]heptan-4-yl)imidazo[1,2-a]pyridine-6-carboxamide

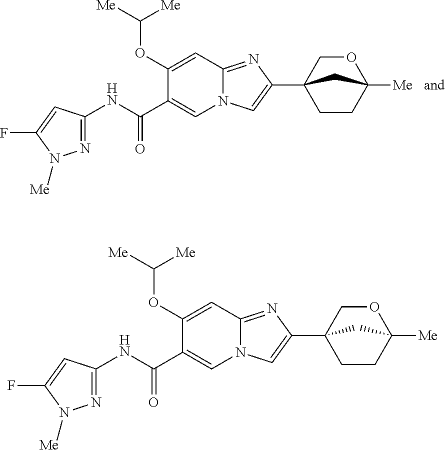 [SMILES: CC(C)Oc1cc2nc([C@]34CC[C@](C)(C3)OC4)cn2cc1C(=O)Nc1cc(F)n(C)n1 ; CC(C)Oc1cc2nc([C@@]34CC[C@@](C)(C3)OC4)cn2cc1C(=O)Nc1cc(F)n(C)n1]

*stereochemistry arbitrarily assigned

N-(5-fluoro-1-methyl-1H-pyrazol-3-yl)-7-isopropoxy-2-((1R,4S)-1-methyl-2-oxabicyclo[2.2.1]heptan-4-yl)imidazo[1,2-a]pyridine-6-carboxamide and N-(5-fluoro-1-methyl-1H-pyrazol-3-yl)-7-isopropoxy-2-((1S,4R)-1-methyl-2-oxabicyclo[2.2.1]heptan-4-yl)imidazo[1,2-a]pyridine-6-carboxamide were obtained from N-(5-fluoro-1-methyl-1H-pyrazol-3-yl)-7-isopropoxy-2-(1-methyl-2-oxabicyclo[2.2.1]heptan-4-yl)imidazo[1,2-a]pyridine-6-carboxamide trifluoroacetate (Example 284) by chiral SFC chromatography (CHIRALPAK IB 30×250 mm 5 um; 30% EtOH+0.1% DEA in CO₂).

*Peak 1, Example 602: LCMS m/z=428.2 [M+H]⁺; ¹H NMR (400 MHz, MeOH-d₄) δ: 1.45-1.53 (m, 3H), 1.57 (d, 6H), 1.83-2.01 (m, 2H), 2.03-2.11 (m, 2H), 2.11-2.30 (m, 2H), 3.70 (d, 3H), 3.93-4.02 (m, 1H), 4.02-4.09 (m, 1H), 5.02-5.14 (m, 1H), 6.33 (d, 1H), 7.32 (s, 1H), 7.96 (s, 1H), 9.14 (s, 1H).

*Peak 2, Example 603: LCMS m/z=428.2 [M+H]⁺; ¹H NMR (400 MHz, MeOH-d₄) δ: 1.45-1.53 (m, 3H), 1.57 (d, 6H), 1.83-2.01 (m, 2H), 2.03-2.11 (m, 2H), 2.11-2.30 (m, 2H), 3.70 (d, 3H), 3.93-4.02 (m, 1H), 4.02-4.09 (m, 1H), 5.02-5.14 (m, 1H), 6.33 (d, 1H), 7.32 (s, 1H), 7.96 (s, 1H), 9.14 (s, 1H).

### Example 604 and Example 605: 8-Ethoxy-N-(2-methoxypyridin-3-yl)-2-((1R,4S)-1-methyl-2-oxabicyclo[2.2.1]heptan-4-yl)imidazo[1,2-a]pyrazine-6-carboxamide and 8-ethoxy-N-(2-methoxypyridin-3-yl)-2-((1S,4R)-1-methyl-2-oxabicyclo[2.2.1]heptan-4-yl)imidazo[1,2-a]pyrazine-6-carboxamide

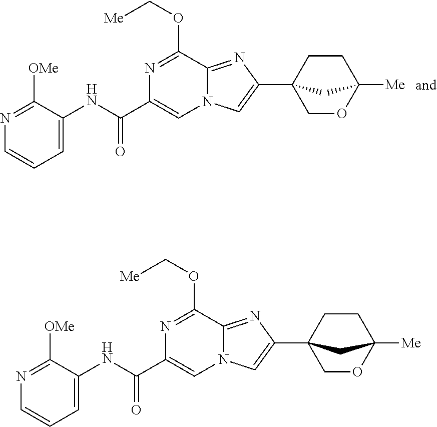 [SMILES: CCOc1nc(C(=O)Nc2cccnc2OC)cn2cc([C@]34CC[C@](C)(C3)OC4)nc12 ; CCOc1nc(C(=O)Nc2cccnc2OC)cn2cc([C@@]34CC[C@@](C)(C3)OC4)nc12]

*stereochemistry arbitrarily assigned

T3P® (50 wt. % in EtOAc, 576 mg, 0.904 mmol) was added to a mixture of 2-methoxypyridin-3-amine (33.7 mg, 0.27 mmol) and 8-ethoxy-2-(1-methyl-2-oxabicyclo[2.2.1]heptan-4-yl)imidazo[1,2-a]pyrazine-6-carboxylic acid (Preparation 335, 57.4 mg, 0.181 mmol) in pyridine (2 mL) and stirred at 22° C. overnight. The mixture was diluted by H₂O and extracted with EtOAc (3×5 mL) and the combined organics dried (MgSO₄) and evaporated to dryness in vacuo. The residue was purified by automated column chromatography (24 g SiO₂, 0-50% 3:1 EtOAc/EtOH in heptane) to afford 8-ethoxy-N-(2-methoxypyridin-3-yl)-2-(1-methyl-2-oxabicyclo[2.2.1]heptan-4-yl)imidazo[1,2-a]pyrazine-6-carboxamide (45.3 mg, 59%) as an off-white solid which was further purified by chiral SFC chromatography to afford 8-ethoxy-N-(2-methoxypyridin-3-yl)-2-((1R,4S)-1-methyl-2-oxabicyclo[2.2.1]heptan-4-yl)imidazo[1,2-a]pyrazine-6-carboxamide and 8-ethoxy-N-(2-methoxypyridin-3-yl)-2-((1S,4R)-1-methyl-2-oxabicyclo[2.2.1]heptan-4-yl)imidazo[1,2-a]pyrazine-6-carboxamide (CHIRALPAK IB 30×250 mm 5 μm; 45% EtOH+0.1% DEA in CO₂)

*Peak 1, Example 604: LCMS m/z=424.2 [M+H]⁺; ¹H NMR (400 MHz, CDCl₃) δ: 1.40-1.52 (m, 3H), 1.66 (t, 3H), 1.79-1.94 (m, 2H), 1.94-2.00 (m, 1H), 2.00-2.08 (m, 1H), 2.08-2.26 (m, 2H), 4.01 (d, 1H), 4.09 (s, 3H), 4.14 (dd, 1H), 4.81 (q, 2H), 6.97 (dd, 1H), 7.51-7.61 (m, 1H), 7.92 (dd, 1H), 8.63 (s, 1H), 8.76 (dd, 1H), 10.15 (s, 1H).

*Peak 2, Example 605: LCMS m/z=424.2 [M+H]⁺; ¹H NMR (400 MHz, CDCl₃) δ: 1.40-1.52 (m, 3H), 1.66 (t, 3H), 1.79-1.94 (m, 2H), 1.94-2.00 (m, 1H), 2.00-2.08 (m, 1H), 2.08-2.26 (m, 2H), 4.01 (d, 1H), 4.09 (s, 3H), 4.14 (dd, 1H), 4.81 (q, 2H), 6.97 (dd, 1H), 7.51-7.61 (m, 1H), 7.92 (dd, 1H), 8.63 (s, 1H), 8.76 (dd, 1H), 10.15 (s, 1H).

### Example 606 and 607: 7-Isopropoxy-N-(1-methyl-1H-pyrazol-3-yl)-2-((1S,4R)-1-methyl-2-oxabicyclo[2.2.1]heptan-4-yl)imidazo[1,2-a]pyridine-6-carboxamide and 7-isopropoxy-N-(1-methyl-1H-pyrazol-3-yl)-2-((1R,4S)-1-methyl-2-oxabicyclo[2.2.1]heptan-4-yl)imidazo[1,2-a]pyridine-6-carboxamide

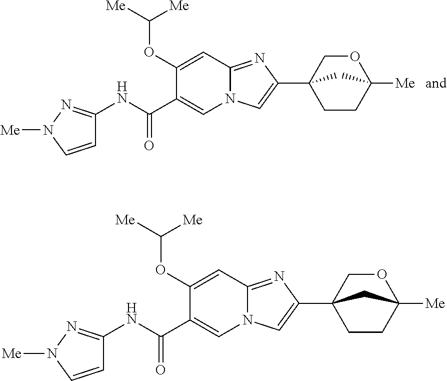 [SMILES: CC(C)Oc1cc2nc([C@@]34CC[C@@](C)(C3)OC4)cn2cc1C(=O)Nc1ccn(C)n1 ; CC(C)Oc1cc2nc([C@]34CC[C@](C)(C3)OC4)cn2cc1C(=O)Nc1ccn(C)n1]

*Stereochemistry was arbitrarily assigned 7-Isopropoxy-N-(1-methyl-1H-pyrazol-3-yl)-2-((1S,4R)-1-methyl-2-oxabicyclo[2.2.1]heptan-4-yl)imidazo[1,2-a]pyridine-6-carboxamide and 7-isopropoxy-N-(1-methyl-1H-pyrazol-3-yl)-2-((1R,4S)-1-methyl-2-oxabicyclo[2.2.1]heptan-4-yl)imidazo[1,2-a]pyridine-6-carboxamide were obtained from 7-isopropoxy-N-(1-methyl-1H-pyrazol-3-yl)-2-(1-methyl-2-oxabicyclo[2.2.1]heptan-4-yl)imidazo[1,2-a]pyridine-6-carboxamide (Example 414) using preparative chiral SFC (CHIRALPAK IB 30×250 mm, 5 μm; 40% MeOH in CO₂).

*Peak 1; Example 606: LCMS m/z=410.2 [M+H]⁺; ¹H NMR (400 MHz, CDCl₃) δ: 1.49 (s, 3H), 1.58 (d, 6H), 1.81-1.90 (m, 2H), 1.90-1.93 (m, 1H), 1.96-2.03 (m, 1H), 2.05-2.21 (m, 2H), 3.84 (s, 3H), 3.97 (d, 1H), 4.10 (dd, 1H), 4.74-4.86 (m, 1H), 6.77 (d, 1H), 6.97 (s, 1H), 7.29 (d, 1H), 7.32 (s, 1H), 9.01 (s, 1H), 10.26 (s, 1H).

*Peak 2; Example 607: LCMS m/z=410.3 [M+H]⁺; ¹H NMR (400 MHz, CDCl₃) δ: 1.49 (s, 3H), 1.58 (d, 6H), 1.80-1.89 (m, 2H), 1.89-1.96 (m, 1H), 1.97-2.03 (m, 1H), 2.07-2.18 (m, 2H), 3.84 (s, 3H), 3.94-4.00 (m, 1H), 3.97 (d, 1H), 4.10 (dd, 1H), 4.79 (dt, 1H), 6.77 (d, 1H), 6.96 (s, 1H), 7.29 (d, 1H), 7.32 (s, 1H), 9.01 (s, 1H), 10.26 (s, 1H).

### Example 608 and 609: N-(6-(difluoromethyl)pyridin-2-yl)-7-isopropoxy-2-((1R,4S)-1-methyl-2-oxabicyclo[2.2.1]heptan-4-yl)imidazo[1,2-a]pyridine-6-carboxamide and N-(6-(difluoromethyl)pyridin-2-yl)-7-isopropoxy-2-((1S,4R)-1-methyl-2-oxabicyclo[2.2.1]heptan-4-yl)imidazo[1,2-a]pyridine-6-carboxamide

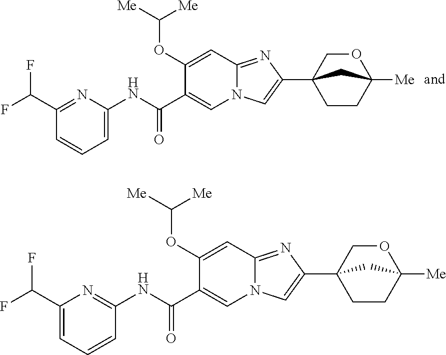 [SMILES: CC(C)Oc1cc2nc([C@]34CC[C@](C)(C3)OC4)cn2cc1C(=O)Nc1cccc(C(F)F)n1 ; CC(C)Oc1cc2nc([C@@]34CC[C@@](C)(C3)OC4)cn2cc1C(=O)Nc1cccc(C(F)F)n1]

T3P® (50 wt. % in EtOAc, 1.07 g, 1.68 mmol) and TEA (730 mg, 7.21 mmol) were added to 7-isopropoxy-2-(1-methyl-2-oxabicyclo[2.2.1]heptan-4-yl)imidazo[1,2-a]pyridine-6-carboxylic acid (Preparation 298, 89.5 mg, 0.200 mmol, 2NaCl) and 6-(difluoromethyl)pyridin-2-amine hydrochloride (39.7 mg, 0.220 mmol) in a microwave vial. The mixture was heated with microwave irritation at 100° C. for 45 min. The reaction mixture was partitioned between EtOAc/brine and the aqueous layer extracted with EtOAc. The combined organics were evaporated to dryness in vacuo. The residue was purified by normal phase column chromatography (SiO₂, 100% EtOAc 100%) to afford N-(6-(difluoromethyl)pyridin-2-yl)-7-isopropoxy-2-(1-methyl-2-oxabicyclo[2.2.1]heptan-4-yl)imidazo[1,2-a]pyridine-6-carboxamide as a white solid (58.8 mg, 64.4%). The sample was purified by preparative chiral SFC (CHIRALPAK IB 30×250 mm, 5 μm; 30% MeOH in CO₂) to afford the title compounds.

*Peak 1, Example 608, N-(6-(difluoromethyl)pyridin-2-yl)-7-isopropoxy-2-((1S,4S)-1-methyl-2-oxabicyclo[2.2.1]heptan-4-yl)imidazo[1,2-a]pyridine-6-carboxamide (13 mg). LCMS m/z=457.2 [M-41]⁺; ¹H NMR (600 MHz, MeOH-d₄) δ: 1.48 (s, 3H), 1.60 (d, 6H), 1.78-2.28 (m, 6H), 3.94 (d, 1H), 4.06 (dd, 1H), 4.98-5.06 (m, 1H), 6.47-6.76 (m, 1H), 6.90-7.05 (m, 1H), 7.45 (d, 1H), 7.63-7.74 (m, 1H), 8.00 (t, 1H), 8.44 (br d, 1H), 9.05-9.18 (m, 1H).

*Peak 2, Example 609, N-(6-(difluoromethyl)pyridin-2-yl)-7-isopropoxy-2-((1R,4R)-1-methyl-2-oxabicyclo[2.2.1]heptan-4-yl)imidazo[1,2-a]pyridine-6-carboxamide (14 mg). LCMS m/z=457.2 [M+H]⁺; ¹H NMR (400 MHz, CDCl₃) δ: 1.49 (s, 3H), 1.60 (d, 6H), 1.77-2.23 (m, 6H), 3.97 (d, 1H), 4.10 (dd, 1H), 4.83 (spt, 1H), 6.33-6.68 (m, 1H), 6.99 (s, 1H), 7.34 (s, 1H), 7.40 (d, 1H), 7.88 (t, 1H), 8.46 (d, 1H), 9.02 (s, 1H), 10.73 (s, 1H).

### Example 610 and 611: 7-Cyclobutoxy-N-(1-methyl-1H-pyrazol-3-yl)-2-((1S,4R)-1-methyl-2-oxabicyclo[2.2.1]heptan-4-yl)imidazo[1,2-a]pyridine-6-carboxamide and 7-cyclobutoxy-N-(1-methyl-1H-pyrazol-3-yl)-2-((1R,4S)-1-methyl-2-oxabicyclo[2.2.1]heptan-4-yl)imidazo[1,2-a]pyridine-6-carboxamide

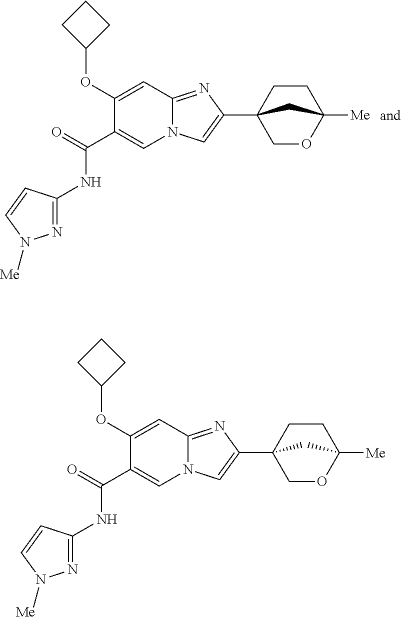 [SMILES: Cn1ccc(NC(=O)c2cn3cc([C@@]45CC[C@@](C)(C4)OC5)nc3cc2OC2CCC2)n1 ; Cn1ccc(NC(=O)c2cn3cc([C@]45CC[C@](C)(C4)OC5)nc3cc2OC2CCC2)n1]

*stereochemistry assigned arbitrarily

7-Cyclobutoxy-N-(1-methyl-1H-pyrazol-3-yl)-2-((1 S,4R)-1-methyl-2-oxabicyclo[2.2.1]heptan-4-yl)imidazo[1,2-a]pyridine-6-carboxamide and 7-cyclobutoxy-N-(1-methyl-1H-pyrazol-3-yl)-2-((1R,4S)-1-methyl-2-oxabicyclo[2.2.1]heptan-4-yl)imidazo[1,2-a]pyridine-6-carboxamide were obtained from 7-cyclobutoxy-N-(1-methyl-1H-pyrazol-3-yl)-2-(1-methyl-2-oxabicyclo[2.2.1]heptan-4-yl)imidazo[1,2-a]pyridine-6-carboxamide (Example 561, 287 mg) by chiral SFC chromatography (CHIRALPAK IB 30×250 mm, 5 um; 40% MeOH in CO₂).

*Peak 1, Example 610, as a white solid (102 mg). LCMS m/z=422.3 [M+H]⁺; ¹H NMR (400 MHz, MeOH-d₄) δ: 1.40-1.53 (m, 3H), 1.74-2.24 (m, 8H), 2.31-2.49 (m, 2H), 2.58-2.76 (m, 2H), 3.79-3.87 (m, 3H), 3.88-3.97 (m, 1H), 4.01-4.11 (m, 1H), 4.96-5.08 (m, 1H), 6.62-6.72 (m, 1H), 6.75-6.84 (m, 1H), 7.46-7.57 (m, 1H), 7.62-7.74 (m, 1H), 8.96-9.11 (m, 1H).

*Peak 2, Example 611, as an off-white solid (109 mg). LCMS m/z=422.3 [M+H]⁺; ¹H NMR (400 MHz, MeOH-d₄) δ: 1.42-1.53 (m, 3H), 1.74-2.25 (m, 8H), 2.31-2.53 (m, 2H), 2.57-2.79 (m, 2H), 3.77-3.88 (m, 3H), 3.90-3.96 (m, 1H), 3.98-4.11 (m, 1H), 4.92-5.08 (m, 1H), 6.62-6.72 (m, 1H), 6.74-6.85 (m, 1H), 7.47-7.61 (m, 1H), 7.63-7.75 (m, 1H), 8.97-9.11 (m, 1H).

### Example 612 and Example 613: 7-Isopropoxy-2-((1S,4R)-1-methyl-2-oxabicyclo[2.2.1]heptan-4-yl)-N-(pyrazolo[1,5-a]pyrimidin-3-yl)imidazo[1,2-a]pyrimidine-6-carboxamide and 7-isopropoxy-2-((1R,4S)-1-methyl-2-oxabicyclo[2.2.1]heptan-4-yl)-N-(pyrazolo[1,5-a]pyrimidin-3-yl)imidazo[1,2-a]pyrimidine-6-carboxamide

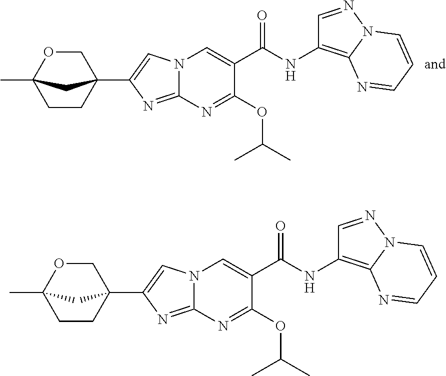 [SMILES: CC(C)Oc1nc2nc([C@@]34CC[C@@](C)(C3)OC4)cn2cc1C(=O)Nc1cnn2cccnc12 ; CC(C)Oc1nc2nc([C@]34CC[C@](C)(C3)OC4)cn2cc1C(=O)Nc1cnn2cccnc12]

*stereochemistry assigned arbitrarily

T3P® (50 wt. % in EtOAc, 845 mg, 1.33 mmol) was added to a mixture of 7-isopropoxy-2-(1-methyl-2-oxabicyclo[2.2.1]heptan-4-yl)imidazo[1,2-a]pyrimidine-6-carboxylic acid (Preparation 345, 88 mg, 0.266 mmol), pyrazolo[1,5-a]pyrimidin-3-amine (49.9 mg, 0.372 mmol) in pyridine (1.8 mL) was added at room temperature and stirred at rt for 2 h. The reaction mixture was diluted with H₂O and extracted with EtOAc (3×). The combined extracts were dried (MgSO₄) and evaporated to dryness in vacuo. The residue was purified by preparative-SFC (CHIRALPAK AD-H 30×250 mm, Sum; 40% EtOH+0.1% DEA in CO₂) the afford 7-isopropoxy-2-((1S,4R)-1-methyl-2-oxabicyclo[2.2.1]heptan-4-yl)-N-(pyrazolo[1,5-a]pyrimidin-3-yl)imidazo[1,2-a]pyrimidine-6-carboxamide and 7-isopropoxy-2-((1R,4S)-1-methyl-2-oxabicyclo[2.2.1]heptan-4-yl)-N-(pyrazolo[1,5-a]pyrimidin-3-yl)imidazo[1,2-a]pyrimidine-6-carboxamide.

*Peak 1, Example 612, 7-isopropoxy-2-((1S,4R)-1-methyl-2-oxabicyclo[2.2.1]heptan-4-yl)-N-(pyrazolo[1,5-a]pyrimidin-3-yl)imidazo[1,2-a]pyrimidine-6-carboxamide (13.6 mg, 11%). LCMS m/z=448.2 [M+H]⁺; ¹H NMR (400 MHz, CDCl₃) δ: 1.50 (s, 3H), 1.67 (d, 7H), 1.80-1.92 (m, 2H), 1.92-1.98 (m, 1H), 1.98-2.06 (m, 1H), 2.06-2.15 (m, 1H), 2.21 (br d, 1H), 3.99 (d, 1H), 4.15 (dd, 1H), 5.88 (dt, 1H), 6.85 (dd, 1H), 8.45 (dd, 1H), 8.64 (dd, 1H), 8.94 (s, 1H), 9.25 (s, 1H), 10.45 (s, 1H),

*Peak 2, Example 613, 7-isopropoxy-2-(1R,4S)-1-methyl-2-oxabicyclo[2.2.1]heptan-4-yl)-N-(pyrazolo[1,5-a]pyrimidin-3-yl)imidazo[1,2-a]pyrimidine-6-carboxamide (13.70 mg, 11%). LCMS m/z=448.2 [M+H]⁺; ¹H NMR (400 MHz, CDCl₃) δ: 1.50 (s, 3H), 1.67 (d, 7H), 1.82-1.92 (m, 2H), 1.92-1.99 (m, 1H), 2.02-2.07 (m, 1H), 2.08-2.16 (m, 1H), 2.20 (br d, 1H), 3.99 (d, 1H), 4.15 (dd, 1H), 5.88 (dt, 1H), 6.85 (dd, 1H), 8.45 (dd, 1H), 8.64 (dd, 1H), 8.94 (s, 1H), 9.25 (s, 1H), 10.45 (s, 1H).

### Example 614 and Example 615: 7-Isopropoxy-2-((1R,4S)-1-methyl-2-oxabicyclo[2.2.1]heptan-4-yl)-N-(pyrazolo[1,5-a]pyrimidin-3-yl)imidazo[1,2-a]pyridine-6-carboxamide and 7-isopropoxy-2-((1S,4R)-1-methyl-2-oxabicyclo[2.2.1]heptan-4-yl)-N-(pyrazolo[1,5-a]pyrimidin-3-yl)imidazo[1,2-a]pyridine-6-carboxamide

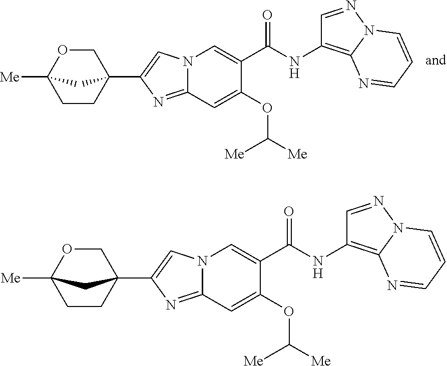 [SMILES: CC(C)Oc1cc2nc([C@]34CC[C@](C)(C3)OC4)cn2cc1C(=O)Nc1cnn2cccnc12 ; CC(C)Oc1cc2nc([C@@]34CC[C@@](C)(C3)OC4)cn2cc1C(=O)Nc1cnn2cccnc12]

*Stereochemistry arbitrarily assigned

7-Isopropoxy-24(1R,4S)-1-methyl-2-oxabicyclo[2.2.1]heptan-4-yl)-N-(pyrazolo[1,5-a]pyrimidin-3-yl)imidazo[1,2-a]pyridine-6-carboxamide and 7-isopropoxy-2-((1S,4R)-1-methyl-2-oxabicyclo[2.2.1]heptan-4-yl)-N-(pyrazolo[1,5-a]pyrimidin-3-yl)imidazo[1,2-a]pyridine-6-carboxamide were obtained from 7-isopropoxy-2-(1-methyl-2-oxabicyclo[2.2.1]heptan-4-yl)-N-(pyrazolo[1,5-a]pyrimidin-3-yl)imidazo[1,2-a]pyridine-6-carboxamide (Example 417) by preparative chiral-SFC chromatography (CHIRALPAK AD-H 30×250 mm 5 μm; 40% MeOH in CO₂).

*Peak 1, Example 614, 16.5 mg, 59%; LCMS m/z=447.2 [M+H]⁺; ¹H NMR (400 MHz, CDCl₃) δ: 1.49 (s, 3H), 1.66 (d, 6H), 1.78-2.22 (m, 6H), 3.97 (d, 1H), 4.11 (dd, 1H), 4.87 (quin, 1H), 6.81 (dd, 1H), 7.01 (s, 1H), 7.35 (s, 1H), 8.40 (dd, 1H), 8.61 (dd, 1H), 8.95 (s, 1H), 9.07 (s, 1H), 10.57 (s, 1H).

*Peak 2, Example 615, 15.5 mg, 55%; LCMS m/z=447.2 [M+H]⁺; ¹H NMR (400 MHz, CDCl₃) δ: 1.49 (s, 3H), 1.66 (d, 6H), 1.77-2.23 (m, 6H), 3.97 (d, 1H), 4.11 (dd, 1H), 4.81-4.93 (m, 1H), 6.81 (dd, 1H), 7.00 (s, 1H), 7.35 (s, 1H), 8.40 (dd, 1H), 8.61 (dd, 1H), 8.95 (s, 1H), 9.07 (s, 1H), 10.57 (s, 1H).

### Example 616 and 617: 7-Isopropoxy-2-((1S,4R)-1-methyl-2-oxabicyclo[2.2.1]heptan-4-yl)-N-(6-methylpyrazolo[1,5-a]pyrimidin-3-yl)imidazo[1,2-a]pyrimidine-6-carboxamide and 7-isopropoxy-2-((1R,4S)-1-methyl-2-oxabicyclo[2.2.1]heptan-4-yl)-N-(6-methylpyrazolo[1,5-a]pyrimidin-3-yl)imidazo[1,2-a]pyrimidine-6-carboxamide

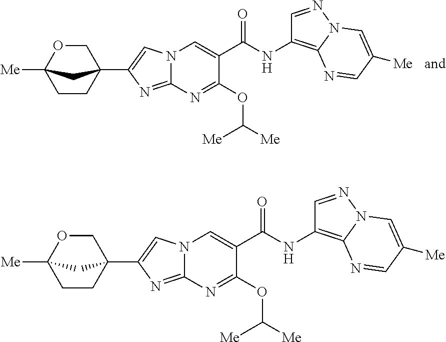 [SMILES: Cc1cnc2c(NC(=O)c3cn4cc([C@@]56CC[C@@](C)(C5)OC6)nc4nc3OC(C)C)cnn2c1 ; Cc1cnc2c(NC(=O)c3cn4cc([C@]56CC[C@](C)(C5)OC6)nc4nc3OC(C)C)cnn2c1]

*stereochemistry assigned arbitrarily

7-Isopropoxy-2-((1S,4R)-1-methyl-2-oxabicyclo[2.2.1]heptan-4-yl)-N-(6-methylpyrazolo[1,5-a]pyrimidin-3-yl)imidazo[1,2-a]pyrimidine-6-carboxamide and 7-isopropoxy-2-((1R,4S)-1-methyl-2-oxabicyclo[2.2.1]heptan-4-yl)-N-(6-methylpyrazolo[1,5-a]pyrimidin-3-yl)imidazo[1,2-a]pyrimidine-6-carboxamide were obtained from 7-isopropoxy-2-(1-methyl-2-oxabicyclo[2.2.1]heptan-4-yl)-N-(6-methylpyrazolo[1,5-a]pyrimidin-3-yl)imidazo[1,2-a]pyrimidine-6-carboxamide (Example 569, 50 mg) by chiral SFC chromatography (CHIRALPAK IB 30×250 mm, 5 μm; 40% MeOH in CO₂).

*Peak 1, Example 616, as a white solid (11.5 mg). LCMS m/z=462.3 [M+H]⁺; ¹H NMR (400 MHz, MeOH-d₄) δ: 1.36 (s, 3H), 1.55 (d, 6H), 1.7-2.1 (m, 6H), 2.30 (d, 3H), 3.82 (d, 1H), 3.95 (dd, 1H), 5.63 (quin, 1H), 7.51 (s, 1H), 8.34 (d, 1H), 8.55 (dd, 1H), 8.58 (s, 1H), 9.27 (s, 1H).

*Peak 2, Example 617, as a white solid (11.9 mg). LCMS m/z=462.3 [M+H]⁺; ¹H NMR (400 MHz, MeOH-d₄) δ: 1.36 (s, 3H), 1.55 (d, 6H), 1.7-2.1 (m, 6H), 2.30 (d, 3H), 3.82 (d, 1H), 3.95 (dd, 1H), 5.63 (quin, 1H), 7.51 (s, 1H), 8.34 (d, 1H), 8.55 (dd, 1H), 8.58 (s, 1H), 9.27 (s, 1H).

### Example 618 and 619: (R)—N-(6-(difluoromethyl)pyridin-2-yl)-2-(1-methyl-2-oxabicyclo[2.1.1]hexan-4-yl)-7-(1,1,1-trifluoropropan-2-yl)oxy)imidazo[1,2-a]pyridine-6-carboxamide and (S)—N-(6-(difluoromethyl)pyridin-2-yl)-2-(1-methyl-2-oxabicyclo[2.1.1]hexan-4-yl)-7-(1,1,1-trifluoropropan-2-yl)oxy)imidazo[1,2-a]pyridine-6-carboxamide

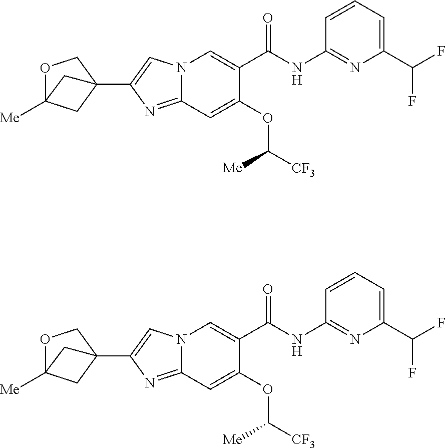 [SMILES: C[C@@H](Oc1cc2nc(C34COC(C)(C3)C4)cn2cc1C(=O)Nc1cccc(C(F)F)n1)C(F)(F)F ; C[C@H](Oc1cc2nc(C34COC(C)(C3)C4)cn2cc1C(=O)Nc1cccc(C(F)F)n1)C(F)(F)F]

*Stereochemistry arbitrarily assigned

N-(6-(difluoromethyl)pyridin-2-yl)-2-(1-methyl-2-oxabicyclo[2.1.1]hexan-4-yl)-7-(1,1,1-trifluoropropan-2-yl)oxy)imidazo[1,2-a]pyridine-6-carboxamide (Example 323) was further purified by SFC (CHIRALPAK IB 30×250 mm, 5 μm: 30% IPA+0.1% DEA in CO₂) to afford (R)—N-(6-(difluoromethyl)pyridin-2-yl)-2-(1-methyl-2-oxabicyclo[2.1.1]hexan-4-yl)-7-((1,1,1-trifluoropropan-2-yl)oxy)imidazo[1,2-a]pyridine-6-carboxamide and (S)—N-(6-(difluoromethyl)pyridin-2-yl)-2-(1-methyl-2-oxabicyclo[2.1.1]hexan-4-yl)-7-(1,1,1-trifluoropropan-2-yl)oxy)imidazo[1,2-a]pyridine-6-carboxamide.

*Peak 1, Example 618: (R)—N-(6-(difluoromethyl)pyridin-2-yl)-2-(1-methyl-2-oxabicyclo[2.1.1]hexan-4-yl)-7-(1,1,1-trifluoropropan-2-yl)oxy)imidazo[1,2-a]pyridine-6-carboxamide (19.3 mg, 14.4%). ¹H NMR (400 MHz, CDCl₃) δ: 1.56 (s, 3H), 1.77 (d, 3H), 2.00 (dd, 2H), 2.12 (br d, 2H), 4.09 (s, 2H), 4.90-5.09 (m, 1H), 6.32-6.72 (m, 1H), 7.21 (br s, 1H), 7.41-7.54 (m, 2H), 7.92 (t, 1H), 8.38-8.53 (m, 1H), 9.09 (s, 1H), 10.05 (s, 1H).

*Peak 2, Example 619: (S)—N-(6-(difluoromethyl)pyridin-2-yl)-2-(1-methyl-2-oxabicyclo[2.1.1]hexan-4-yl)-7-(1,1,1-trifluoropropan-2-yl)oxy)imidazo[1,2-a]pyridine-6-carboxamide (16.5 mg, 12.3%). ¹H NMR (400 MHz, CDCl₃) δ: 1.56 (s, 3H), 1.77 (d, 3H), 1.97-2.01 (m, 2H), 2.12 (br s, 2H), 4.09 (s, 2H), 4.98 (br s, 1H), 6.36-6.68 (m, 1H), 7.15 (br s, 1H), 7.42-7.49 (m, 2H), 7.92 (t, 1H), 8.45 (d, 1H), 9.09 (s, 1H), 10.05 (s, 1H)

### Example 620 and 621: 7-Isopropoxy-N-(1-methyl-2-oxo-1,2-dihydropyridin-3-yl)-2-((1S,4R)-1-methyl-2-oxabicyclo[2.2.1]heptan-4-yl)imidazo[1,2-a]pyridine-6-carboxamide and 7-isopropoxy-N-(1-methyl-2-oxo-1,2-dihydropyridin-3-yl)-2-((1R,4S)-1-methyl-2-oxabicyclo[2.2.1]heptan-4-yl)imidazo[1,2-a]pyridine-6-carboxamide

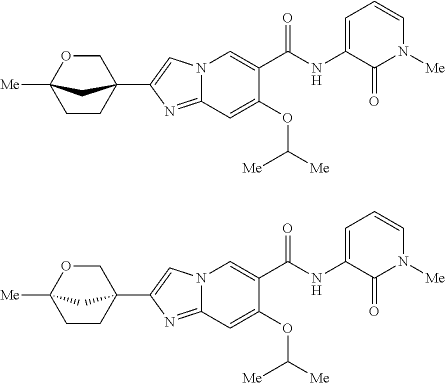 [SMILES: CC(C)Oc1cc2nc([C@@]34CC[C@@](C)(C3)OC4)cn2cc1C(=O)Nc1cccn(C)c1=O ; CC(C)Oc1cc2nc([C@]34CC[C@](C)(C3)OC4)cn2cc1C(=O)Nc1cccn(C)c1=O]

*Stereochemistry arbitrarily assigned

To a mixture of 7-isopropoxy-2-(1-methyl-2-oxabicyclo[2.2.1]heptan-4-yl)imidazo[1,2-a]pyridine-6-carboxylic acid (Preparation 298, 150 mg, 0.454 mmol) and 3-amino-1-methyl-pyridin-2-one (73.3 mg, 0.590 mmol) in pyridine (1.14 mL) was added T3P® (50 wt. % in EtOAc, 1.44 g, 2.27 mmol) and the reaction stirred at rt for 2 h. The mixture was partitioned between EtOAc and water, the layers separated and the aqueous extracted with EtOAc (3×). The combined organic layers were dried (MgSO₄), filtered, and concentrated in vacuo. The crude was purified by SFC (CHIRALPAK IB 30×250 mm, 5 um, Method: 40% EtOH+0.1% DEA in CO₂) to provide two enantiomers. Each was further purified by column chromatography on silica gel eluting with 0-60% 3:1 EtOAc:EtOH in heptanes to afford 7-Isopropoxy-N-(1-methyl-2-oxo-1,2-dihydropyridin-3-yl)-2-((1S,4R)-1-methyl-2-oxabicyclo[2.2.1]heptan-4-yl)imidazo[1,2-a]pyridine-6-carboxamide and 7-isopropoxy-N-(1-methyl-2-oxo-1,2-dihydropyridin-3-yl)-2-((1R,4S)-1-methyl-2-oxabicyclo[2.2.1]heptan-4-yl)imidazo[1,2-a]pyridine-6-carboxamide.

*Peak 1, Example 620, 55.3 mg, 28%; LCMS m/z=437.3 [M+H]⁺; ¹H NMR (400 MHz, CDCl₃) δ: 1.49 (s, 3H), 1.64 (d, 6H), 1.82-1.90 (m, 2H), 1.94 (s, 1H), 1.99-2.06 (m, 1H), 2.06-2.23 (m, 2H), 3.65 (s, 3H), 3.97 (d, 1H), 4.04-4.12 (m, 1H), 4.78-4.87 (m, 1H), 6.26 (t, 1H), 6.98-7.06 (m, 2H), 7.32 (s, 1H), 8.59 (dd, 1H), 8.99 (s, 1H), 10.81 (s, 1H).

*Peak 2, Example 621, 52.4 mg, 26%; ¹H NMR (400 MHz, CDCl₃) δ: 1.49 (s, 3H), 1.56-1.71 (m, 6H), 1.78-1.95 (m, 3H), 1.97-2.04 (m, 1H), 2.06-2.20 (m, 2H), 3.65 (s, 3H), 3.97 (d, 1H), 4.09-4.15 (m, 1H), 4.85 (spt, 1H), 6.26 (t, 1H), 7.05 (d, 1H), 7.26-7.39 (m, 2H), 8.59 (dd, 1H), 8.99 (s, 1H), 10.81 (s, 1H).

### Example 622 and 623: 8-Cyclobutoxy-2-((1S,4R)-1-methyl-2-oxabicyclo[2.2.1]heptan-4-yl)-N-(pyridin-2-yl)imidazo[1,2-a]pyridine-6-carboxamide and 8-cyclobutoxy-2-((1R,4S)-1-methyl-2-oxabicyclo[2.2.1]heptan-4-yl)-N-(pyridin-2-yl)imidazo[1,2-a]pyridine-6-carboxamide

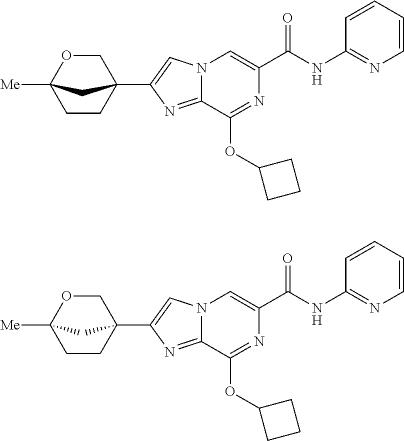 [SMILES: C[C@@]12CC[C@@](c3cn4cc(C(=O)Nc5ccccn5)nc(OC5CCC5)c4n3)(CO1)C2 ; C[C@]12CC[C@](c3cn4cc(C(=O)Nc5ccccn5)nc(OC5CCC5)c4n3)(CO1)C2]

*Stereochemistry arbitrarily assigned

To a mixture of 8-cyclobutoxy-2-(1-methyl-2-oxabicyclo[2.2.1]heptan-4-yl)imidazo[1,2-a]pyrazine-6-carboxylic acid (Preparation 369, 100 mg, 291 mmol) and pyridin-2-amine (32.9 mg, 0.349 mmol) in pyridine (1.46 mL) was added T3P® (50 wt. % in EtOAc, 927 mg, 1.46 mmol) and the reaction stirred at rt for 2 h. The mixture was partitioned between EtOAc and water, the layers separated and the aqueous extracted with EtOAc (3×). The combined organic layers were dried (MgSO₄), filtered, and concentrated in vacuo. The crude was purified by SFC (CHIRALPAK IB 30×250 mm 5 μm: 45% MeOH+0.1% DEA in CO₂) to afford the 8-Cyclobutoxy-2-((1S,4R)-1-methyl-2-oxabicyclo[2.2.1]heptan-4-yl)-N-(pyridin-2-yl)imidazo[1,2-a]pyridine-6-carboxamide and 8-cyclobutoxy-2-((1R,45)-1-methyl-2-oxabicyclo[2.2.1]heptan-4-yl)-N-(pyridin-2-yl)imidazo[1,2-a]pyridine-6-carboxamide.

*Peak 1, Example 622, 21 mg, 17%; ¹H NMR (500 MHz, CDCl₃) δ: 1.47-1.53 (m, 3H), 1.79-2.03 (m, 5H), 2.04-2.09 (m, 1H), 2.12-2.25 (m, 2H), 2.47-2.57 (m, 2H), 2.65-2.74 (m, 2H), 4.02 (d, 1H), 4.15 (dd, 1H), 5.57 (br s, 1H), 7.10-7.19 (m, 1H), 7.53-7.60 (m, 1H), 7.82 (br t, 1H), 8.37-8.48 (m, 2H), 8.65-8.71 (m, 1H), 10.03 (br s, 1H)

*Peak 2, Example 623, 22.4 mg, 18%; ¹H NMR (500 MHz, CDCl₃) δ: 1.50 (s, 3H), 1.79-1.98 (m, 5H), 2.04-2.09 (m, 1H), 2.10-2.27 (m, 2H), 2.45-2.59 (m, 2H), 2.61-2.77 (m, 2H), 4.02 (d, 1H), 4.15 (dd, 1H), 5.48-5.65 (m, 1H), 7.05-7.19 (m, 1H), 7.51-7.60 (m, 1H), 7.75-7.85 (m, 1H), 8.34-8.47 (m, 2H), 8.63-8.70 (m, 1H), 9.99 (br s, 1H)

### Example 624 and 625: N-(1-(difluoromethyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide and N-(1-(difluoromethyl)-2-oxo-1,2-dihydropyridin-3-yl)-7-isopropoxy-2-((1R,45)-1-methyl-2-oxabicyclo[2.2.1]heptan-4-yl)imidazo[1,2-a]pyridine-6-carboxamide

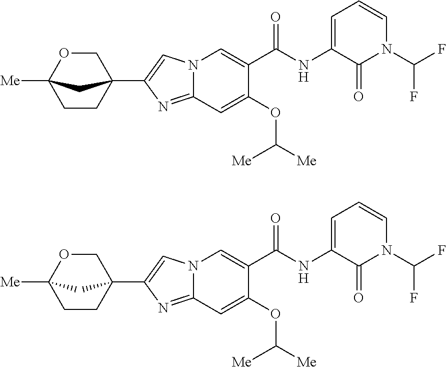 [SMILES: CC(C)Oc1cc2nc([C@@]34CC[C@@](C)(C3)OC4)cn2cc1C(=O)Nc1cccn(C(F)F)c1=O ; CC(C)Oc1cc2nc([C@]34CC[C@](C)(C3)OC4)cn2cc1C(=O)Nc1cccn(C(F)F)c1=O]

*Stereochemistry arbitrarily assigned

N-(1-(difluoromethyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide and N-(1-(difluoromethyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyridine-6-carboxamide were obtained from 7-isopropoxy-2-(1-methyl-2-oxabicyclo[2.2.1]heptan-4-yl)imidazo[1,2-a]pyridine-6-carboxylic acid (Preparation 298) and 3-amino-1-(difluoromethyl)pyridin-2-one following the procedure described in Example 622 and 623.

*Peak 1, Example 624, LCMS m/z=473.2 [M+H]⁺; ¹H NMR (400 MHz, CDCl₃) δ: 1.41 (s, 3H), 1.53 (d, 6H), 1.73-1.82 (m, 2H), 1.84-1.88 (m, 1H), 1.90-1.97 (m, 1H), 1.99-2.10 (m, 2H), 3.88 (d, 1H), 3.97-4.00 (m, 1H), 4.81 (spt, 1H), 6.32 (t, 1H), 7.15 (dd, 1H), 7.23 (d, 1H), 7.52-7.89 (m, 1H), 8.13 (s, 1H), 8.53 (dd, 1H), 8.90 (s, 1H), 10.69 (s, 1H).

*Peak 2, Example 625, 37.3 mg, 17.4%; ¹H NMR (400 MHz, CDCl₃) δ: 1.40 (s, 3H), 1.53 (d, 6H), 1.74-1.80 (m, 2H), 1.82-1.89 (m, 1H), 1.90-1.93 (m, 1H), 1.97-2.13 (m, 2H), 3.88 (d, 1H), 3.99 (dd, 1H), 4.81 (spt, 1H), 6.32 (t, 1H), 7.15 (dd, 1H), 7.22 (d, 1H), 7.52-7.90 (m, 1H), 8.15 (br s, 1H), 8.52 (dd, 1H), 8.90 (s, 1H), 10.69 (s, 1H).

### Example 626 and 627: 8-Cyclobutoxy-N-(6-(difluoromethyl)pyridin-2-yl)-2-((1S,4R)-1-methyl-2-oxabicyclo[2.2.1]heptan-4-yl)imidazo[1,2-a]pyrazine-6-carboxamide and 8-cyclobutoxy-N-(6-(difluoromethyl)pyridin-2-yl)-2-((1R,4S)-1-methyl-2-oxabicyclo[2.2.1]heptan-4-yl)imidazo[1,2-a]pyrazine-6-carboxamide

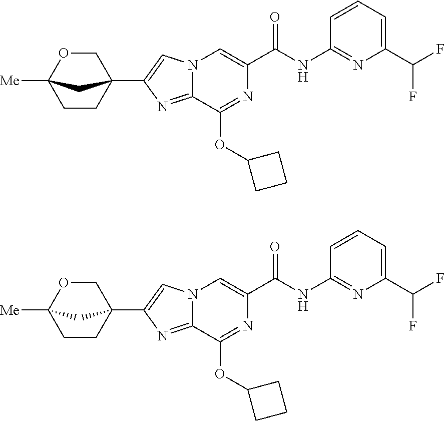 [SMILES: C[C@@]12CC[C@@](c3cn4cc(C(=O)Nc5cccc(C(F)F)n5)nc(OC5CCC5)c4n3)(CO1)C2 ; C[C@]12CC[C@](c3cn4cc(C(=O)Nc5cccc(C(F)F)n5)nc(OC5CCC5)c4n3)(CO1)C2]

*Stereochemistry arbitrarily assigned

8-Cyclobutoxy-N-(6-(difluoromethyl)pyridin-2-yl)-24(1S,4R)-1-methyl-2-oxabicyclo[2.2.1]heptan-4-yl)imidazo[1,2-a]pyrazine-6-carboxamide and 8-cyclobutoxy-N-(6-(difluoromethyl)pyridin-2-yl)-2-((1R,4S)-1-methyl-2-oxabicyclo[2.2.1]heptan-4-yl)imidazo[1,2-a]pyrazine-6-carboxamide were obtained from 8-cyclobutoxy-2-(1-methyl-2-oxabicyclo[2.2.1]heptan-4-yl)imidazo[1,2-a]pyrazine-6-carboxylic acid (Preparation 369) and 6-(difluoromethyl)pyridin-2-amine following the procedure described in Example 622 and 623.

*Peak 1, Example 626, 30.4 mg, 22%; ¹H NMR (400 MHz, CDCl₃) δ: 1.50 (s, 3H), 1.79-1.94 (m, 3H), 1.96-2.03 (m, 2H), 2.03-2.11 (m, 1H), 2.12-2.25 (m, 2H), 2.48-2.60 (m, 2H), 2.66-2.74 (m, 2H), 4.02 (d, 1H), 4.15 (dd, 1H), 5.53 (quin, 1H), 6.41-6.74 (m, 1H), 7.45 (d, 1H), 7.55-7.61 (m, 1H), 7.93 (t, 1H), 8.51 (d, 1H), 8.68 (s, 1H), 10.00 (s, 1H).

*Peak 2, Example 627, 28.4 mg, 20.6%; ¹H NMR (400 MHz, CDCl₃) δ: 1.51 (s, 3H), 1.79-1.92 (m, 3H), 1.95-2.01 (m, 2H), 2.01-2.09 (m, 1H), 2.10-2.27 (m, 2H), 2.43-2.62 (m, 2H), 2.64-2.76 (m, 2H), 4.03 (d, 1H), 4.15 (dd, 1H), 5.53 (quin, 1H), 6.43-6.77 (m, 1H), 7.45 (d, 1H), 7.51-7.60 (m, 1H), 7.93 (t, 1H), 8.46-8.56 (m, 1H), 8.68 (s, 1H), 10.00 (s, 1H).

### Example 628 and 629: N-(1-(difluoromethyl)-2-oxo-1,2-dihydropyridin-3-yl)-7-isopropoxy-2-((1 S,4R)-1-methyl-2-oxabicyclo[2.2.1]heptan-4-yl)imidazo[1,2-a]pyrimidine-6-carboxamide and N-(1-(difluoromethyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyrimidine-6-carboxamide

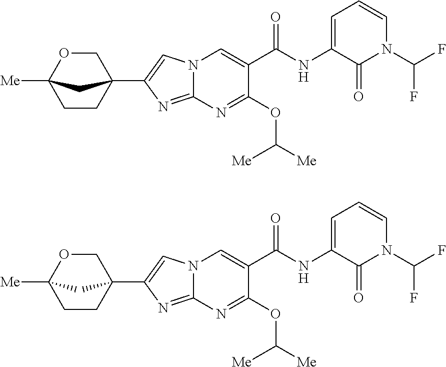 [SMILES: CC(C)Oc1nc2nc([C@@]34CC[C@@](C)(C3)OC4)cn2cc1C(=O)Nc1cccn(C(F)F)c1=O ; CC(C)Oc1nc2nc([C@]34CC[C@](C)(C3)OC4)cn2cc1C(=O)Nc1cccn(C(F)F)c1=O]

*Stereochemistry arbitrarily assigned

N-(1-(difluoromethyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyrimidine-6-carboxamide and N-(1-(difluoromethyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyrimidine-6-carboxamide were obtained from 7-isopropoxy-2-(1-methyl-2-oxabicyclo[2.2.1]heptan-4-yl)imidazo[1,2-a]pyrimidine-6-carboxylic acid (Preparation 345) and 3-amino-1-(difluoromethyl)pyridin-2-one hydrochloride, following a similar method to that described in Examples 622 and 623, but using the following SFC conditions (CHIRALPAK AD-H 30×250 mm 5 μm: 40% EtOH+0.1% DEA in CO₂)

*Peak 1, Example 628, 27.6 mg, 21.7%; LCMS m/z=474.2 [M+H]⁺; ¹H NMR (400 MHz, CDCl₃) δ: 1.45-1.53 (m, 3H), 1.61 (d, 6H), 1.80-1.90 (m, 2H), 1.91-1.99 (m, 1H), 2.00-2.05 (m, 1H), 2.06-2.13 (m, 1H), 2.15-2.25 (m, 1H), 3.97 (d, 1H), 4.14 (dd, 1H), 5.89 (spt, 1H), 6.42 (t, 1H), 7.27 (d, 2H), 7.64-8.03 (m, 1H), 8.60 (dd, 1H), 9.16 (s, 1H), 10.79 (s, 1H).

*Peak 2, Example 629, 28.5 mg, 22.4%; ¹H NMR (400 MHz, CDCl₃) δ: 1.50 (s, 3H), 1.61 (d, 6H), 1.79-1.91 (m, 2H), 1.91-1.99 (m, 1H), 2.01-2.06 (m, 1H), 2.06-2.13 (m, 1H), 2.18-2.26 (m, 1H), 3.97 (d, 1H), 4.14 (dd, 1H), 5.89 (spt, 1H), 6.42 (t, 1H), 7.27 (d, 2H), 7.64-8.00 (m, 1H), 8.60 (dd, 1H), 9.16 (s, 1H), 10.79 (s, 1H).

### Example 630 and 631: N-(6-fluoropyrazolo[1,5-a]pyrimidin-3-yl)-7-isopropoxy-2-((1S,4R)-1-methyl-2-oxabicyclo[2.2.1]heptan-4-yl)imidazo[1,2-a]pyrimidine-6-carboxamide and N-(6-fluoropyrazolo[1,5-a]pyrimidin-3-yl)-7-isopropoxy-2-((1R,4S)-1-methyl-2-oxabicyclo[2.2.1]heptan-4-yl)imidazo[1,2-a]pyrimidine-6-carboxamide

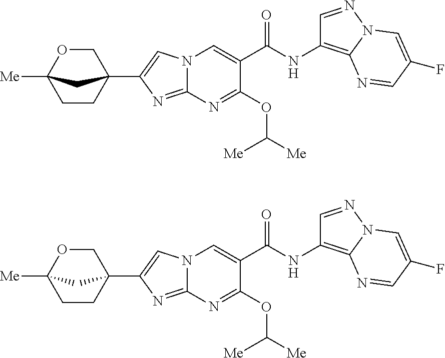 [SMILES: CC(C)Oc1nc2nc([C@@]34CC[C@@](C)(C3)OC4)cn2cc1C(=O)Nc1cnn2cc(F)cnc12 ; CC(C)Oc1nc2nc([C@]34CC[C@](C)(C3)OC4)cn2cc1C(=O)Nc1cnn2cc(F)cnc12]

*Stereochemistry arbitrarily assigned

N-(6-fluoropyrazolo[1,5-a]pyrimidin-3-yl)-7-isopropoxy-2-((1S,4R)-1-methyl-2-oxabicyclo[2.2.1]heptan-4-yl)imidazo[1,2-a]pyrimidine-6-carboxamide and N-(6-fluoropyrazolo[1,5-a]pyrimidin-3-yl)-7-isopropoxy-2-((1R,4S)-1-methyl-2-oxabicyclo[2.2.1]heptan-4-yl)imidazo[1,2-a]pyrimidine-6-carboxamide were obtained from 7-isopropoxy-2-(1-methyl-2-oxabicyclo[2.2.1]heptan-4-yl)imidazo[1,2-a]pyrimidine-6-carboxylic acid (Preparation 345) and 6-fluoropyrazolo[1,5-a]pyrimidin-3-amine, following a similar method to that described in Example 622 and 623, except using the following SFC conditions (CHIRALPAK AD-H 30×250 mm 5 μm: 40% EtOH+0.1% DEA in CO₂).

*Peak 1, Example 630, 54.1 mg, 21.2%; LCMS m/z=466.2 [M+H]⁺; ¹H NMR (400 MHz, CDCl₃) δ: 1.47-1.51 (m, 3H), 1.65 (d, 6H), 1.81-1.91 (m, 2H), 1.91-1.96 (m, 1H), 2.00-2.05 (m, 1H), 2.06-2.12 (m, 1H), 2.15-2.24 (m, 1H), 3.98 (d, 1H), 4.15 (dd, 1H), 5.86 (spt, 1H), 7.29 (s, 1H), 8.49 (d, 1H), 8.60 (dd, 1H), 8.93 (s, 1H), 9.24 (s, 1H), 10.46 (s, 1H).

*Peak 2, Example 631, 51.8 mg, 20.3%; ¹H NMR (400 MHz, CDCl₃) δ: 1.47-1.54 (m, 3H), 1.66 (d, 6H), 1.79-1.91 (m, 2H), 1.94 (d, 1H), 2.00-2.13 (m, 2H), 2.15-2.25 (m, 1H), 3.98 (d, 1H), 4.15 (dd, 1H), 5.86 (spt, 1H), 7.28 (s, 1H), 8.49 (d, 1H), 8.60 (dd, 1H), 8.94 (s, 1H), 9.24 (s, 1H), 10.47 (s, 1H).

### Example 632: 7-(Cyclopentyloxy)-N-(1-methyl-1H-pyrazol-3-yl)-2-(1-methyl-2-oxabicyclo[2.2.1]heptan-4-yl)imidazo[1,2-a]pyridine-6-carboxamide

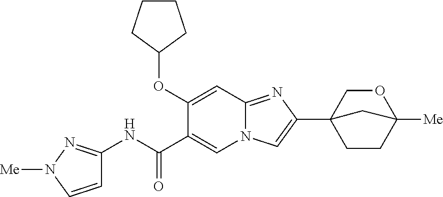 [SMILES: Cn1ccc(NC(=O)c2cn3cc(C45CCC(C)(C4)OC5)nc3cc2OC2CCCC2)n1]

HATU (224.6 mg, 0.589 mmol), DIPEA (145 mg, 1.12 mmol) and 1-methylpyrazol-3-amine (65.4 mg, 0.673 mmol) were added to a solution of 7-cyclopentoxy-2-(1-methyl-2-oxabicyclo[2.2.1]heptan-4-yl)imidazo[1,2-a]pyridine-6-carboxylic acid (Preparation 328, 200 mg, 0.561 mmol) in DMF (5 mL) and the reaction stirred at rt overnight. The reaction was purified by prep-HPLC-F to afford 7-(cyclopentyloxy)-N-(1-methyl-1H-pyrazol-3-yl)-2-(1-methyl-2-oxabicyclo[2.2.1]heptan-4-yl)imidazo[1,2-a]pyridine-6-carboxamide. LCMS m/z=436.3 (M+H)⁺; ¹H NMR (500 MHz, MeOH-d₄) δ: 1.38 (s, 3H), 1.62-1.72 (m, 3H), 1.74-1.85 (m, 5H), 1.85-1.96 (m, 3H), 1.99-2.11 (m, 3H), 3.17 (d, 2H), 3.71-3.80 (m, 4H), 3.87-3.97 (m, 1H), 4.10 (q, 1H), 5.08-5.18 (m, 1H), 6.58 (d, 1H), 7.07 (s, 1H), 7.63 (d, 1H), 7.74 (s, 1H), 9.01 (s, 1H), 10.29 (s, 1H).

### Example 633 and 634: 7-(Cyclopentyloxy)-N-(1-methyl-1H-pyrazol-3-yl)-2-((1R,4S)-1-methyl-2-oxabicyclo[2.2.1]heptan-4-yl)imidazo[1,2-a]pyridine-6-carboxamide and 7-(cyclopentyloxy)-N-(1-methyl-1H-pyrazol-3-yl)-2-((1S,4R)-1-methyl-2-oxabicyclo[2.2.1]heptan-4-yl)imidazo[1,2-a]pyridine-6-carboxamide

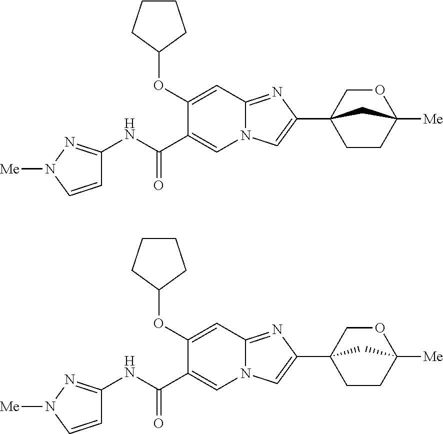 [SMILES: Cn1ccc(NC(=O)c2cn3cc([C@]45CC[C@](C)(C4)OC5)nc3cc2OC2CCCC2)n1 ; Cn1ccc(NC(=O)c2cn3cc([C@@]45CC[C@@](C)(C4)OC5)nc3cc2OC2CCCC2)n1]

*Stereochemistry arbitrarily assigned.

7-(Cyclopentyloxy)-N-(1-methyl-1H-pyrazol-3-yl)-2-((1R,4S)-1-methyl-2-oxabicyclo[2.2.1]heptan-4-yl)imidazo[1,2-a]pyridine-6-carboxamide and 7-(cyclopentyloxy)-N-(1-methyl-1H-pyrazol-3-yl)-2-((1S,4R)-1-methyl-2-oxabicyclo[2.2.1]heptan-4-yl)imidazo[1,2-a]pyridine-6-carboxamide were obtained from 7-(cyclopentyloxy)-N-(1-methyl-1H-pyrazol-3-yl)-2-(1-methyl-2-oxabicyclo[2.2.1]heptan-4-yl)imidazo[1,2-a]pyridine-6-carboxamide (Example 632) by SFC (CHIRALPAK AD-H 30×250 mm 5 μm; 40% IPA+0.1% DEA in CO₂).

*Peak 1, Example 634, LCMS m/z=436.3 [M+H]⁺; ¹H NMR (500 MHz, MeOH-d₄) δ: 1.46 (s, 3H), 1.70-2.22 (m, 14H), 3.83 (s, 3H), 3.92 (d, 1H), 4.01-4.07 (m, 1H), 5.09-5.21 (m, 1H), 6.65 (d, 1H), 6.94 (s, 1H), 7.50 (d, 1H), 7.66 (s, 1H), 9.02 (s, 1H),

*Peak 2, Example 633, LCMS m/z=436.3 [M+H]⁺; ¹H NMR (500 MHz, MeOH-d₄) δ: 1.46 (s, 3H), 1.70-2.00 (m, 8H), 2.02-2.22 (m, 6H), 3.83 (s, 3H), 3.91 (d, 1H), 4.01-4.07 (m, 1H), 5.09-5.18 (m, 1H), 6.65 (d, 1H), 6.93 (s, 1H), 7.50 (d, 1H), 7.66 (s, 1H), 9.02 (s, 1H)

### Example 635 and 636: 7-(Cyclopentyloxy)-N-(1-methyl-1H-pyrazol-3-yl)-2-((1S,4R)-1-methyl-2-oxabicyclo[2.2.1]heptan-4-yl)imidazo[1,2-a]pyrimidine-6-carboxamide and 7-(cyclopentyloxy)-N-(1-methyl-1H-pyrazol-3-yl)-2-((1R,4S)-1-methyl-2-oxabicyclo[2.2.1]heptan-4-yl)imidazo[1,2-a]pyrimidine-6-carboxamide

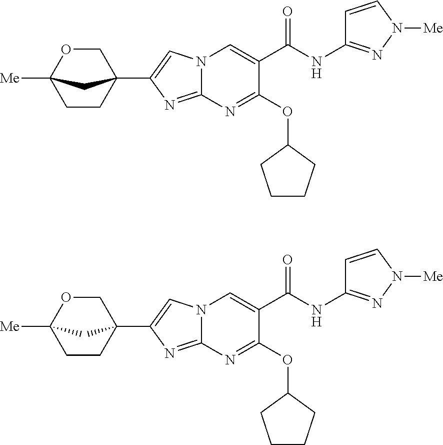 [SMILES: Cn1ccc(NC(=O)c2cn3cc([C@@]45CC[C@@](C)(C4)OC5)nc3nc2OC2CCCC2)n1 ; Cn1ccc(NC(=O)c2cn3cc([C@]45CC[C@](C)(C4)OC5)nc3nc2OC2CCCC2)n1]

*Stereochemistry arbitrarily assigned.

To 7-cyclopentyloxy-2-(1-methyl-2-oxabicyclo[2.2.1]heptan-4-yl)imidazo[1,2-a]pyridine-6-carboxylic acid (Preparation 328, 150 mg, 419.70 umol) and 1-methylpyrazol-3-amine (61.14 mg, 629.55 umol) in DMF (1.4 mL) was added HATU (176.01 mg, 461.67 umol) and DIPEA (216.97 mg, 1.68 mmol) and the reaction stirred at rt for 3 h. The reaction was partitioned between EtOAc and brine, the layers separated and the aqueous layer was extracted with EtOAc (3×). The combined organic were dried (MgSO₄), and evaporated to dryness in vacuo. The residue was purified by column chromatography on silica gel eluting with (0-60% 3:1 EtOAc:EtOH in heptanes) and further purified by SFC (CHIRALPAK IB 30×250 mm, 5 μm: 40% EtOH+0.1% DEA in CO₂) to afford 7-(cyclopentyloxy)-N-(1-methyl-1H-pyrazol-3-yl)-2-((1S,4R)-1-methyl-2-oxabicyclo[2.2.1]heptan-4-yl)imidazo[1,2-a]pyrimidine-6-carboxamide and 7-(cyclopentyloxy)-N-(1-methyl-1H-pyrazol-3-yl)-2-((1R,4S)-1-methyl-2-oxabicyclo[2.2.1]heptan-4-yl)imidazo[1,2-a]pyrimidine-6-carboxamide.

*Peak 1, Example 635, 7.8 mg, 4.21%; LCMS m/z=437.3 [M+H]⁺; ¹H NMR (400 MHz, CDCl₃) δ: 1.34-1.43 (m, 2H), 1.49 (s, 3H), 1.80-1.88 (m, 2H), 1.92-1.97 (m, 2H), 2.00-2.13 (m, 5H), 2.16 (br dd, 2H), 3.10-3.14 (m, 1H), 3.86 (s, 3H), 3.97 (d, 1H), 4.13 (dd, 1H), 5.86-5.98 (m, 1H), 6.74 (d, 1H), 7.27-7.32 (m, 2H), 9.19 (s, 1H), 10.08 (s, 1H).

*Peak 2, Example 636, 6.6 mg, 3.6%; ¹H NMR (400 MHz, CDCl₃) δ: 1.34-1.43 (m, 2H), 1.50 (s, 3H), 1.78-1.89 (m, 3H), 1.91-1.97 (m, 2H), 2.00-2.10 (m, 4H), 2.12-2.26 (m, 3H), 3.03-3.10 (m, 1H), 3.86 (s, 3H), 3.97 (d, 1H), 4.14 (dd, 1H), 5.92 (tt, 1H), 6.75 (d, 1H), 7.26-7.32 (m, 3H), 9.18 (s, 1H), 10.09 (s, 1H)

### Example 637: 2-Cyclopropyl-8-(2-fluoroethoxy)-N-(1-methyl-2-oxo-1,2-dihydropyridin-3-yl)imidazo[1,2-a]pyrazine-6-carboxamide trifluoroacetate

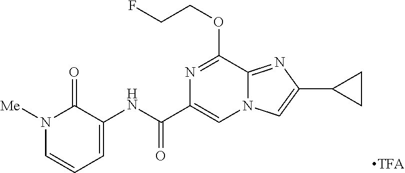 [SMILES: Cn1cccc(NC(=O)c2cn3cc(C4CC4)nc3c(OCCF)n2)c1=O]

1-fluoro-2-iodo-ethane (26 mg, 0.149 mmol) was added in one portion to a suspension of 2-cyclopropyl-8-hydroxy-N-(1-methyl-2-oxo-1,2-dihydropyridin-3-yl)imidazo[1,2-a]pyrazine-6-carboxamide (Preparation 394, 44.2 mg, 0.136 mmol) and K₂CO₃ (37.6 mg, 0.271 mmol) in DMF (2 mL) and the mixture heated at 50° C. for 2 h. The reaction was quenched with saturated aqueous NH₄Cl solution and extracted with EtOAc (3×5 mL). The combined organics were dried (MgSO₄) and evaporated to dryness in vacuo. The residue was purified by prep-HPLC-D to afford 2-cyclopropyl-8-(2-fluoroethoxy)-N-(1-methyl-2-oxo-1,2-dihydropyridin-3-yl)imidazo[1,2-a]pyrazine-6-carboxamide trifluoroacetate. LCMS m/z=372.4 [M+H]⁺; ¹H NMR (500 MHz, DMSO-d₆) δ: 0.79-0.92 (m, 2H), 0.92-1.05 (m, 2H), 2.07-2.21 (m, 1H), 3.47-3.72 (m, 1H), 3.57 (s, 1H), 4.73-4.82 (m, 1H), 4.82-4.88 (m, 1H), 4.90 (dd, 1H), 5.00 (dd, 1H), 6.32-6.42 (m, 1H), 7.49 (dd, 1H), 7.99-8.12 (m, 1H), 8.38 (dd, 1H), 8.87-9.01 (m, 1H), 10.45 (s, 1H).

### Example 638: N-(6-cyanopyridin-2-yl)-2-cyclopropyl-7-isopropoxyimidazo[1,2-a]pyridine-6-carboxamide

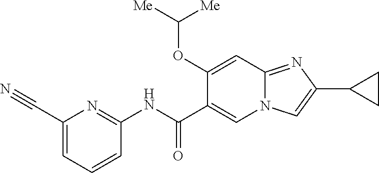 [SMILES: CC(C)Oc1cc2nc(C3CC3)cn2cc1C(=O)Nc1cccc(C#N)n1]

DABAl-Me3 (28.37 mg, 0.111 mmol) was added to a solution of methyl 2-cyclopropyl-7-isopropoxyimidazo[1,2-a]pyridine-6-carboxylate (Preparation 254, 37.95 mg, 0.138 mmol) and 6-aminopyridine-2-carbonitrile (16.48 mg, 0.138 mmol) in THF (0.5 mL) and the mixture purged with Ar for 30 seconds and then heated at 160° C. under mW conditions for 5 min. The reaction was quenched with sat aq NaHCO₃ (15 mL) and extracted with DCM (20 mL). The combined organics were washed with brine (2×20 mL), dried (Na₂SO₄) and evaporated to dryness in vacuo. The residue was purified by prep-HPLC-J to afford N-(6-cyanopyridin-2-yl)-2-cyclopropyl-7-isopropoxyimidazo[1,2-a]pyridine-6-carboxamide (5.3 mg, 10.6%). LCMS m/z=362.0 [M+H]⁺; ¹H NMR (CDCl₃, 400 MHz): δ: 0.94-1.01 (m, 4H), 1.58 (d, 6H), 1.96-2.02 (m, 1H), 4.81 (p, 1H), 6.93 (s, 1H), 7.31 (s, 1H), 7.43 (d, 1H), 7.83 (t, 1H), 8.59 (d, 1H), 8.93 (s, 1H), 10.78 (s, 1H).

### Example 639: 7-(Cyclopropylmethoxy)-N-(1-(difluoromethyl)-1H-pyrazol-3-yl)-2-(1-methyl-2-oxabicyclo[2.1.1]hexan-4-yl)imidazo[1,2-a]pyrimidine-6-carboxamide

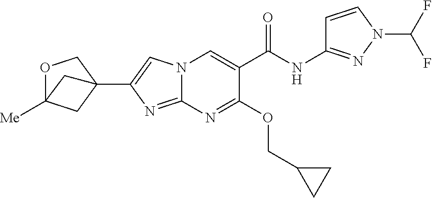 [SMILES: CC12CC(c3cn4cc(C(=O)Nc5ccn(C(F)F)n5)c(OCC5CC5)nc4n3)(CO1)C2]

To a solution of 7-(cyclopropylmethoxy)-2-(1-methyl-2-oxabicyclo[2.1.1]hexan-4-yl)imidazo[1,2-a]pyrimidine-6-carboxylic acid (Preparation 314, 100 mg, 0.30 mmol), 1-(difluoromethyl)-1H-pyrazol-3-amine (83.3 mg, 0.62 mmol) and HATU (139 mg, 0.36 mmol) in DCM (2 mL) was added TEA (61.5 mg, 0.62 mmol) and the reaction stirred at 20° C. for 14 h. The mixture was concentrated in vacuo and the residue purified by prep-HPLC-F to afford 7-(cyclopropylmethoxy)-N-(1-(difluoromethyl)-1H-pyrazol-3-yl)-2-(1-methyl-2-oxabicyclo[2.1.1]hexan-4-yl)imidazo[1,2-a]pyrimidine-6-carboxamide (15 mg, 11%). LCMS m/z=445.1 [M+H]⁺; ¹H NMR (500 MHz, MeOH-d₄) δ: 0.51-0.53 (m, 2H), 0.71-0.74 (m, 2H), 1.47-1.51 (m, 4H), 1.85-1.86 (m, 2H), 2.09-2.12 (m, 2H), 3.99 (s, 2H), 4.49 (d, 2H), 6.98 (d, 1H), 7.40 (t, 1H), 7.61 (s, 1H), 7.99 (s, 1H), 9.31 (s, 1H)

### Example 640: 7-Isopropoxy-N-(6-(isoxazol-4-yl)pyridin-2-yl)-2-(1-methyl-2-oxabicyclo[2.1.1]hexan-4-yl)imidazo[1,2-a]pyrimidine-6-carboxamide

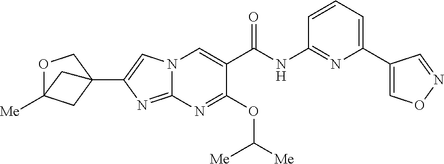 [SMILES: CC(C)Oc1nc2nc(C34COC(C)(C3)C4)cn2cc1C(=O)Nc1cccc(-c2cnoc2)n1]

Part A: To a solution of 7-isopropoxy-2-(1-methyl-2-oxabicyclo[2.1.1]hexan-4-yl)imidazo[1,2-a]pyrimidine-6-carboxylic acid (Preparation 128, 70.6 mg, 0.189 mmol) and 6-bromopyridin-2-amine (98.1 mg, 0.567 mmol) in pyridine (4 mL) was added T3P® (50% in EtOAc, 4 mL) and the reaction stirred at 20° C. for 16 h. The mixture was concentrated in vacuo and the residue neutralised with sat. aq.NaHCO₃ and the aqueous solution extracted with EtOAc (3×50 mL). The combined organics were washed with brine (50 mL), dried (Na₂SO₄) and evaporated to dryness in vacuo. The residue was purified by chromatography (PE/EtOAc=1/1) to give N-(6-bromopyridin-2-yl)-7-isopropoxy-2-(1-methyl-2-oxabicyclo[2.1.1]hexan-4-yl)imidazo[1,2-a]pyrimidine-6-carboxamide as a white solid (30 mg, 26%) which was used in Part B below. LCMS m/z=473.8 [M+H]⁺

Part B: To a solution of N-(6-bromopyridin-2-yl)-7-isopropoxy-2-(1-methyl-2-oxabicyclo[2.1.1]hexan-4-yl)imidazo[1,2-a]pyrimidine-6-carboxamide (30 mg, 0.0635 mmol) in dioxane (5 mL) and water (1 mL) was added 4-(4,4,5,5-tetramethyl-1,3,2-dioxaborolan-2-yl)isoxazole (12.4 mg, 0.0635 mmol), K₃PO₄ (40.5 mg, 0.191 mmol) and Pd(dppf)Cl₂ (4.65 mg, 0.00635 mmol) under N₂ and the reaction stirred at 90° C. for 2 h. The mixture was diluted with water (10 mL), extracted with EtOAc (20 mL×2) and the combined organic layers washed with brine (20 mL), dried over Na₂SO₄ and filtered. The filtrate was concentrated in vacuo and the residue purified by prep-HPLC-J to afford 7-isopropoxy-N-(6-(isoxazol-4-yl)pyridin-2-yl)-2-(1-methyl-2-oxabicyclo[2.1.1]hexan-4-yl)imidazo[1,2-a]pyrimidine-6-carboxamide as a white solid (5.0 mg, 17.1%). LCMS m/z=461.1 [M+H]⁺¹H NMR (500 MHz, MeOH-d₄) δ: 1.50 (s, 3H), 1.65 (d, 6H), 1.90-1.84 (m, 2H), 2.10-2.15 (m, 2H), 4.01 (s, 2H), 5.64-5.73 (m, 1H), 7.52 (d, 1H), 7.63 (s, 1H), 7.88 (t, 1H), 8.23 (d, 1H), 8.90 (s, 1H), 9.23 (s, 1H), 9.39 (s, 1H)

### Example 641: N-(5-fluoro-1-methyl-1H-pyrazol-3-yl)-7-isopropoxy-2-(1-methyl-2-oxabicyclo[2.1.1]hexan-4-yl)imidazo[1,2-a]pyridine-6-carboxamide trifluoroacetate

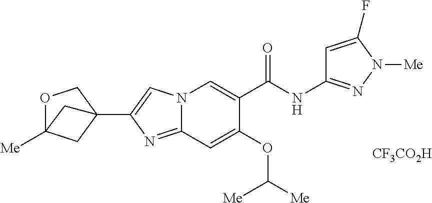 [SMILES: CC(C)Oc1cc2nc(C34COC(C)(C3)C4)cn2cc1C(=O)Nc1cc(F)n(C)n1 ; "CF3CO2H"]

Using an analogous method to that described for Example 252 using 7-isopropoxy-2-(1-methyl-2-oxabicyclo[2.1.1]hexan-4-yl)imidazo[1,2-a]pyridine-6-carboxylic acid (Preparation 78) and 5-fluoro-1-methyl-1H-pyrazol-3-amine hydrochloride afforded N-(5-fluoro-1-methyl-1H-pyrazol-3-yl)-7-isopropoxy-2-(1-methyl-2-oxabicyclo[2.1.1]hexan-4-yl)imidazo[1,2-a]pyridine-6-carboxamide trifluoroacetate (54.4 mg, 32.6%). LCMS m/z=414.2 [M+H]⁺; ¹H NMR (400 MHz, CDCl₃) δ: 1.56 (s, 3H), 1.63 (d, 6H), 2.01 (dd, 2H), 2.27 (br s, 2H), 3.70 (d, 3H), 4.08 (s, 2H), 5.00 (spt, 1H), 6.35 (d, 1H), 7.37 (s, 1H), 7.76 (br s, 1H), 9.09 (s, 1H), 10.06 (s, 1H).

### Examples 642 and 643: rel-(R)-7-(sec-butoxy)-2-(1-methyl-2-oxabicyclo[2.1.1]hexan-4-yl)-N-(6-methylpyrazolo[1,5-a]pyrimidin-3-yl)imidazo[1,2-a]pyrimidine-6-carboxamide and rel-(S)-7-(sec-butoxy)-2-(1-methyl-2-oxabicyclo[2.1.1]hexan-4-yl)-N-(6-methylpyrazolo[1,5-a]pyrimidin-3-yl)imidazo[1,2-a]pyrimidine-6-carboxamide

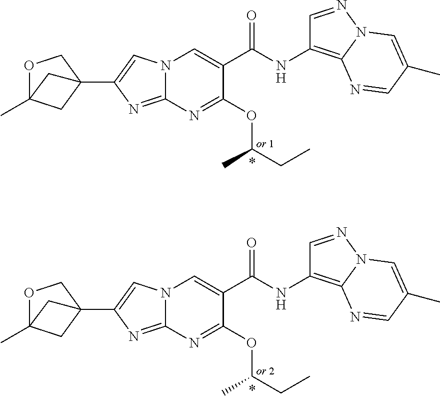

*Stereochemistry arbitrarily assigned

To a solution of 6-methylpyrazolo[1,5-a]pyrimidin-3-amine (71.5 mg, 483 μmol, 2.0 eq.) in pyridine (2 mL) was added rac-(R)-7-(sec-butoxy)-2-(1-methyl-2-oxabicyclo[2.1.1]hexan-4-yl)imidazo[1,2-a]pyrimidine-6-carboxylic acid (preparation 399, 80.0 mg, 241 μmol, 1.0 eq.) and T3P (2 mL) at 20° C. The reaction was stirred at 20° C. for 2 hours. The reaction was evaporated under vacuum. The residue was diluted with aqueous NaHCO₃ (30 mL), extracted with EtOAc (30 mL×3). The organic layer was dried over Na₂SO₄; filtered and evaporated under vacuum. The residue was purified by Combi-Flash (PE: EA from 1:1 to 0:1) to give racemic title compound (100 mg, 80.7% yield) as a yellow solid, which was purified by prep-SFC (Column: DAICEL CHIRALPAK IG (250 mm×30 mm, 10 um); Mobile Phase: from 60% to 60% of 0.1% NH₃H₂O ETOH; Flow Rate (ml/min): 80; Column temp: 35° C.) to give two enantiomers.

*Peak 1, Example 642; 31.9 mg, 31.90% yield; LCMS: m/z=462.0 [M+H]⁺. ¹H NMR: (500 MHz, CDCl₃) δ: 1.07 (t, J=7.5 Hz, 3H), 1.54 (s, 3H), 1.59 (d, J=6.0 Hz, 3H), 1.95-1.90 (m, 1H), 1.97-1.95 (m, 2H), 2.12-2.10 (m, 2H), 2.15-2.12 (m, 1H), 2.41 (s, 3H), 4.09 (s, 2H), 5.74-5.69 (m, 1H), 7.30 (s, 1H), 8.32 (d, J=1.5 Hz, 1H), 8.42 (s, 1H), 8.83 (s, 1H), 9.24 (s, 1H), 10.43 (brs, 1H).

*Peak 2, Example 643; 26.9 mg, 26.90% yield; LCMS: m/z=462.0 [M+H]⁺. ¹H NMR: (500 MHz, CDCl₃) δ: 1.07 (t, J=7.5 Hz, 3H), 1.54 (s, 3H), 1.59 (d, J=6.0 Hz, 3H), 1.95-1.90 (m, 1H), 1.97-1.95 (m, 2H), 2.12-2.09 (m, 2H), 2.15-2.12 (m, 1H), 2.41 (s, 3H), 4.09 (s, 2H), 5.74-5.69 (m, 1H), 7.30 (s, 1H), 8.32 (d, J=1.5 Hz, 1H), 8.42 (s, 1H), 8.83 (s, 1H), 9.24 (s, 1H), 10.42 (brs, 1H).

### Examples 644 and 645: rel-(R)-7-(sec-butoxy)-2-(1-(fluoromethyl)-2-oxabicyclo[2.1.1]hexan-4-yl)-N-(6-methylpyrazolo[1,5-a]pyrimidin-3-yl)imidazo[1,2-a]pyrimidine-6-carboxamide and rel-(S)-7-(sec-butoxy)-2-(1-(fluoromethyl)-2-oxabicyclo[2.1.1]hexan-4-yl)-N-(6-methylpyrazolo[1,5-a]pyrimidin-3-yl)imidazo[1,2-a]pyrimidine-6-carboxamide

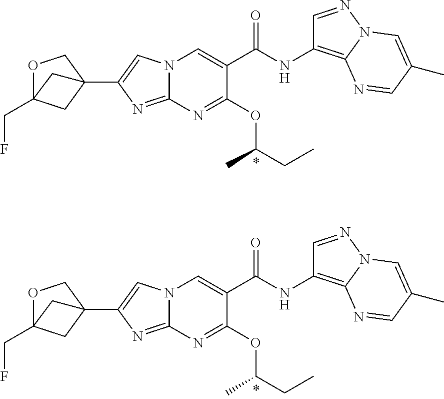 [SMILES: CC[C@@H](C)Oc1nc2nc(C34COC(CF)(C3)C4)cn2cc1C(=O)Nc1cnn2cc(C)cnc12 ; CC[C@H](C)Oc1nc2nc(C34COC(CF)(C3)C4)cn2cc1C(=O)Nc1cnn2cc(C)cnc12]

*Stereochemistry arbitrarily assigned

To a solution of rac-(R)-7-(sec-butoxy)-2-(1-(fluoromethyl)-2-oxabicyclo[2.1.1]hexan-4-yl)imidazo[1,2-a]pyrimidine-6-carboxylic acid (preparation 402, 76.3 mg, 515 μmol, 2.0 eq.) in pyridine (2 mL) was added compound 5 (90.0 mg, 257 μmol, 1.0 eq.) and T3P (2 mL) at 20° C. The reaction was stirred at 20° C. for 2 hours. The reaction was evaporated under vacuum. The residue was diluted with aqueous NaHCO₃ (30 mL), extracted with EtOAc (30 mL×2). The organic layer was dried over Na₂SO₄; filtered and evaporated under vacuum. The residue was purified by Combi-Flash (PE: EA from 1:1 to 0:1) to give racemic title compound (100 mg, 72.8% yield) as a yellow solid, which was purified by prep-SFC (Column: DAICEL CHIRALPAK IG (250 mm×30 mm, 10 um); Mobile Phase: from 60% to 60% of 0.1% NH₃H₂O ETOH; Flow Rate (ml/min): 80; Column temp: 35° C.) to give two enantiomers.

*Peak 1, Example 644; 49.9 mg, 49.9% yield; LCMS: m/z=480.0 [M+H]⁺. ¹H NMR: (500 MHz, CDCl₃) δ: 1.08 (t, J=7.5 Hz, 3H), 1.60 (d, J=6.5 Hz, 3H), 1.96-1.90 (m, 1H), 2.08-2.06 (m, 2H), 2.17-2.09 (m, 1H), 2.28-2.27 (m, 2H), 2.41 (s, 3H), 4.16 (s, 2H), 4.76-4.66 (m, 2H), 5.74-5.70 (m, 1H), 7.33 (s, 1H), 8.33 (d, J=2.0 Hz, 1H), 8.42 (s, 1H), 8.83 (s, 1H), 9.25 (s, 1H), 10.42 (brs, 1H).

*Peak 2, Example 645; 44.7 mg, 44.7%; LCMS: m/z=480.1 [M+H]⁺. ¹H NMR: (500 MHz, CDCl₃) δ: 1.08 (t, J=7.5 Hz, 3H). 1.60 (d, J=6.0 Hz, 3H), 1.96-1.90 (m, 1H), 2.08-2.06 (m, 2H), 2.17-2.09 (m, 1H), 2.28-2.26 (m, 2H), 2.41 (s, 3H), 4.16 (s, 2H), 4.76-4.66 (m, 2H), 5.74-5.69 (m, 1H), 7.33 (s, 1H), 8.33 (d, J=2.0 Hz, 1H), 8.42 (s, 1H), 8.83 (s, 1H), 9.25 (s, 1H), 10.42 (brs, 1H).

### Example 646: rel-2-((1R,5R)-2,6-dioxabicyclo[3.2.1]octan-1-yl)-7-isopropoxy-N-(6-methylpyrazolo[1,5-a]pyrimidin-3-yl)imidazo[1,2-a]pyridine-6-carboxamide

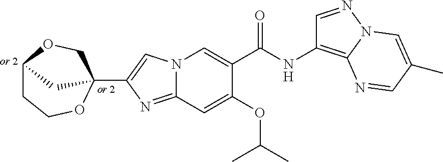 [SMILES: Cc1cnc2c(NC(=O)c3cn4cc([C@]56CO[C@H](CCO5)C6)nc4cc3OC(C)C)cnn2c1]

To a solution of rac-24(1R,5R)-2,6-dioxabicyclo[3.2.1]octan-1-yl)-7-isopropoxyimidazo[1,2-a]pyridine-6-carboxylic acid (preparation 404, 50.0 mg, 0.150 mmol, 1.0 eq.) and 6-methylpyrazolo[1,5-a]pyrimidin-3-amine (44.6 mg, 0.301 mmol, 2.0 eq.) in Pyridine (1.00 mL) was added T3P (1.00 mL). The mixture was stirred at 20° C. for 16 h. The reaction mixture was concentrated to give the residue. The residue was diluted with water (10 mL) and adjusted by aqueous NaHCO₃ (10 mL) and extracted with EA (20 mL×3). The combined organic layer was washed with brine (30 mL), dried over Na₂SO₄, The mixture was filtered and the filtrate was purified by prep-HPLC (Column:Phenomenex Synergi C18 150*30 mm*4 um; Mobile Phase: from 49% to 69% of water (0.05% (NH₄HCO₃)-ACN) to the racemic title compound (50.0 mg, 71.9% yield) as a white solid, which was purified by SFC (Column: ChiralPak OJ—3 100*4.6 mm I.D., 3 μm; Mobile phase: A: CO₂ B: iso-propanol (0.05% DEA); Isocratic: 40% B; Flow rate: 2.8 mL/min; Column temp: 35° C.; Back pressure: 100 bar) to give rel-2-((1R,5R)-2,6-dioxabicyclo[3.2.1]octan-1-yl)-7-isopropoxy-N-(6-methylpyrazolo[1,5-a]pyrimidin-3-yl)imidazo[1,2-a]pyridine-6-carboxamide (20.3 mg, 40.6% yield) as a yellow solid. LCMS: m/z=463.1 [M+H]⁺. ¹H NMR: (400 MHz, MeOH-d₄) δ: 1.64 (d, J=6.0 Hz, 6H), 1.77-1.67 (m, 1H), 1.87 (td, J=12.0 6.5 Hz, 1H), 2.06 (d, J=10.5 Hz, 1H), 2.39 (s, 3H), 2.55-2.45 (m, 1H), 4.24-3.93 (m, 3H), 4.35 (d, J=9.51 Hz, 1H), 4.81-4.65 (m, 1H), 5.02 (dt, J=12.0 6.0 Hz, 1H), 7.00 (s, 1H), 7.78 (s, 1H), 8.41 (d, J=2.0 Hz, 1H), 8.61 (s, 1H), 8.68 (s, 1H), 9.13 (s, 1H).

### Example 647: rel-2-((1R,5R)-2,6-dioxabicyclo[3.2.1]octan-1-yl)-7-isopropoxy-N-(6-methylpyrazolo[1,5-a]pyrimidin-3-yl)imidazo[1,2-a]pyrimidine-6-carboxamide

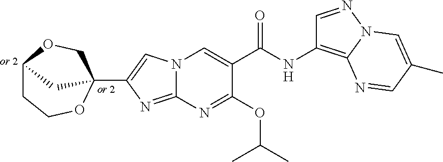 [SMILES: Cc1cnc2c(NC(=O)c3cn4cc([C@]56CO[C@H](CCO5)C6)nc4nc3OC(C)C)cnn2c1]

To a solution of rac-24(1R,5R)-2,6-dioxabicyclo[3.2.1]octan-1-yl)-7-isopropoxyimidazo[1,2-a]pyrimidine-6-carboxylic acid (preparation 407, 80.0 mg, 240 μmol, 1 eq.) and 6-methylpyrazolo[1,5-a]pyrimidin-3-amine (53.3 mg, 360 μmol, 1.5 eq.) in Pyridine (3 mL) was added T3P (3 mL). The mixture was stirred at 20° C. for 30 min. The mixture was concentrated in vacuo to give the residue, which was diluted with saturated NaHCO₃aq. till pH=7. And this mixture was extracted with EtOAc (50 mL×3). The combined organic layer was washed with brine (50 mL) and dried over Na₂SO₄, filtered. The filtrate was concentrated in vacuo to give the residue, which was purified by Combi-Flash (PE/EtOAc=0/1) to give racemic title compound (106 mg, 95.3% yield) as a yellow solid, which was purified by SFC (Column: Chiralcel OJ-3 100ᵢÀ4.6 mm I.D., 3 um; Mobile phase: A: CO₂ B:ethanol (0.05% DEA); Isocratic: 40% B; Flow rate: 2.8 mL/min; Column temp.: 35° C.; ABPR: 1500 psi) to give rel-2-((1R,5R)-2,6-dioxabicyclo[3.2.1]octan-1-yl)-7-isopropoxy-N-(6-methylpyrazolo[1,5-a]pyrimidin-3-yl)imidazo[1,2-a]pyrimidine-6-carboxamide (38.96 mg, 36.64% yield) as a yellow solid. LCMS: m/z=464.3 [M+H]⁺. ¹H NMR: (400 MHz, CDCl₃) δ: 1.65 (d, J=6.0 Hz, 6H), 1.89-1.79 (m, 2H), 2.01 (d, J=11.2 Hz, 1H), 2.41 (s, 3H), 2.68-2.63 (m, 1H), 4.12-4.06 (m, 1H), 4.27-4.19 (m, 1H), 4.32 (s, 2H), 4.77-4.74 (m, 1H), 5.88-5.81 (m, 1H), 7.48 (s, 1H), 8.33 (s, 1H), 8.42 (s, 1H), 8.83 (s, 1H), 9.24 (s, 1H), 10.42 (s, 1H).

### Example 648: rel-7-Cyclobutoxy-N-(1-methyl-1H-pyrazol-3-yl)-2-((1R,4S)-1-methyl-2-oxabicyclo[2.2.1]heptan-4-yl)imidazo[1,2-a]pyrimidine-6-carboxamide

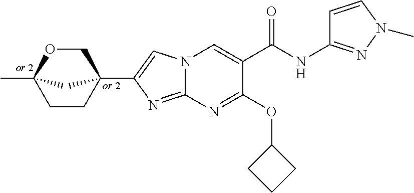

To a solution of 2-amino-4-cyclobutoxy-N-(1-methyl-1H-pyrazol-3-yl)pyrimidine-5-carboxamide (preparation 411, 72.8 mg, 312 μmol, 1.0 eq.) and rac-2-bromo-1-((1R,4R)-1-methyl-2-oxabicyclo[2.2.1]heptan-4-yl)ethan-1-one (60.0 mg, 208 μmol, 1.5 eq.) in tBuOH (2.00 mL) was added Na₂CO₃ (66.2 mg, 624 μmol, 3.0 eq.). Then, the reaction mixture was stirred under 80° C. for 16 hours. The reaction mixture was extracted with EtOAc (50 mL×3). Then the combined organic layer was dried with Na₂SO₄ and concentrated in vacuum to give the residue. The residue was purified by column chromatography on silica gel (from PE: EA=1:1) to give racemic title compound as a white solid, which was further purified by prep. SFC (Column: ChiralCel OD-3 (150×4.6 mm, 3 μm), 40° C.; Mobile Phase: 50% of 0.05% DEA IPA) to give rel-7-cyclobutoxy-N-(1-methyl-1H-pyrazol-3-yl)-2-((1R,4S)-1-methyl-2-oxabicyclo[2.2.1]heptan-4-yl)imidazo[1,2-a]pyrimidine-6-carboxamide (19.4 mg, 21.8% yield) as a white solid. LCMS: m/z=423.3 [M+H]⁺. ¹H NMR: (500 MHz, CDCl₃) δ: 1.48 (s, 3H), 1.89-1.72 (m, 3H), 1.93-1.90 (m, 1H), 1.97-1.94 (m, 1H), 2.02-1.99 (m, 1H), 2.10-2.03 (m, 1H), 2.20-2.14 (m, 1H), 2.39-2.30 (m, 2H), 2.72-2.66 (m, 2H), 3.86 (s, 3H), 3.95 (d, J=6.5 Hz, 1H), 4.13-4.10 (m, 1H), 5.62-5.55 (m, 1H), 6.76 (d, J=2.5 Hz, 1H), 7.23 (s, 1H), 7.31 (d, J=2.0 Hz, 1H), 9.17 (s, 1H), 10.06 (s, 1H).

### Example 651: 7-Cyclobutoxy-N-(1-cyclopropyl-2-oxo-1,2-dihydropyridin-3-yl)-2-(1-methyl-2-oxabicyclo[2.1.1]hexan-4-yl)imidazo[1,2-a]pyrimidine-6-carboxamide

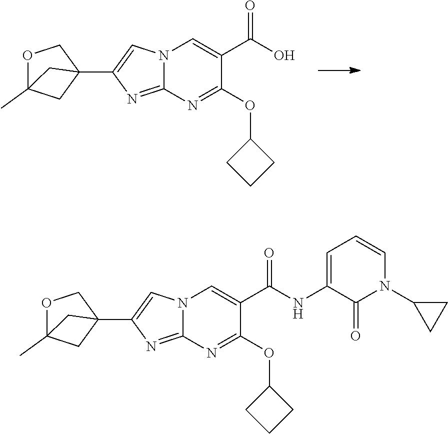 [SMILES: CC12CC(c3cn4cc(C(=O)O)c(OC5CCC5)nc4n3)(CO1)C2 ; CC12CC(c3cn4cc(C(=O)Nc5cccn(C6CC6)c5=O)c(OC5CCC5)nc4n3)(CO1)C2]

To a mixture of 7-cyclobutoxy-2-(1-methyl-2-oxabicyclo[2.1.1]hexan-4-yl)imidazo[1,2-a]pyrimidine-6-carboxylic acid (Preparation 313, 60 mg, 0.18 mmol) and 3-amino-1-cyclopropylpyridin-2(1H)-one (38 mg, 0.25 mmol) in pyridine (1 mL) was added a solution of T3P® (50 wt. % in EtOAc) (0.91 mmol, 0.5 mL, 50% purity) at rt. After stirring for 2 h, the reaction mixture was diluted with water, extracted with DCM and EtOAc, dried over MgSO₄, filtered, and concentrated. The crude material was purified by mass-directed reverse-phase HPLC [XSelect CSH Prep C18 Sum OBD 19×100 mm]eluting with Mobile phase A: MeCN; Mobile phase B: H₂O, Modifier: 0.1% NH₄OH to provide 7-cyclobutoxy-N-(1-cyclopropyl-2-oxo-1,2-dihydropyridin-3-yl)-2-(1-methyl-2-oxabicyclo[2.1.1]hexan-4-yl)imidazo[1,2-a]pyrimidine-6-carboxamide (24.6 mg, 29% yield). m/z 462.0 (M+H)+, δ 1H NMR (500 MHz, DMSO-d₆) δ ppm 0.90-0.94 (m, 2H) 1.03-1.08 (m, 2H) 1.43 (s, 3H) 1.73-1.80 (m, 3H) 1.90-1.97 (m, 1H) 1.99 (dd, J=4.58, 1.53 Hz, 2H) 2.52-2.58 (m, 5H) 3.87 (s, 2H) 5.36-5.52 (m, 1H) 6.31 (t, J=7.02 Hz, 1H) 7.35 (dd, J=7.32, 1.83 Hz, 1H) 7.72 (s, 1H) 8.44 (dd, J=7.32, 1.83 Hz, 1H) 9.48 (s, 1H) 10.71 (s, 1H).

### Example 652 and Example 653: 7-isopropoxy-N-(1-methyl-2-oxo-1,2-dihydropyridin-3-yl)-2-((1S,4R)-1-methyl-2-oxabicyclo[2.2.1]heptan-4-yl)imidazo[1,2-a]pyrimidine-6-carboxamide and 7-isopropoxy-N-(1-methyl-2-oxo-1,2-dihydropyridin-3-yl)-2-((1R,4S)-1-methyl-2-oxabicyclo[2.2.1]heptan-4-yl)imidazo[1,2-a]pyrimidine-6-carboxamide

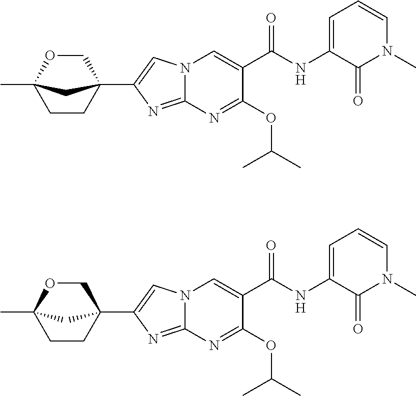

*Stereochemistry arbitrarily assigned

To a solution of 7-isopropoxy-2-(1-methyl-2-oxabicyclo[2.2.1]heptan-4-yl)imidazo[1,2-a]pyrimidine-6-carboxylic acid (Preparation 345, 150 mg, 0.453 mmol) and 3-amino-1-methylpyridin-2(1H)-one (112 mg, 0.91 mmol) in Pyridine (3.0 mL, 37 mmol) was added a solution of T3P® (50 wt. % in EtOAc)® (3.0 mL, 5 mmol, 50% purity). The mixture was stirred at 20° C. for 2 h. The reaction mixture was concentrated and the residue was diluted with a mixture of water (10 mL) and aqueous NaHCO₃ (10 mL) and extracted with EtOAc (20 mL×3). The combined organic layers were washed with brine (30 mL), dried (Na₂SO₄), and filtered. The filtrate was purified by prep-HPLC (Column:Phenomenex Synergi C18 150*30 mm*4 um; Mobile Phase: eluting with 49%-69% water (0.05% (NH₄HCO₃)-ACN) to give 7-isopropoxy-N-(1-methyl-2-oxo-1,2-dihydropyridin-3-yl)-2-(1-methyl-2-oxabicyclo[2.2.1]heptan-4-yl)imidazo[1,2-a]pyrimidine-6-carboxamide (90.0 mg, 0.195 mmol, 43.1% yield, 95% purity) as a racemic white solid. LCMS m/z=438.3 [M+H]⁺. The racemic product was purified by SFC (Column: Phenomenex—Cellulose-2 (250 mm*30 mm, 5 um); Mobile phase: A: CO₂ B: iso-propanol (0.05% DEA); Isocratic: 60% B; Flow rate: 2.8 mL/min; Column temp.: 35° C.; Back pressure: 1500 psi) to give Example 652 (Peak 1, 35.2 mg, 44.0% yield, >99% ee) and Example 653 (Peak 2, 28.4 mg, 35.5% yield, >99% ee) as yellow solids.

*Peak 1, Example 652: 7-isopropoxy-N-(1-methyl-2-oxo-1,2-dihydropyridin-3-yl)-2-((1S,4R)-1-methyl-2-oxabicyclo[2.2.1]heptan-4-yl)imidazo[1,2-a]pyrimidine-6-carboxamide, 35.2 mg, ¹H NMR: (500 MHz, Methanol-d4) δ ppm=9.34 (s, 1H), 8.53 (d, J=7.5 Hz, 1H), 7.56 (s, 1H), 7.33 (d, J=7.0 Hz, 1H), 6.34 (t, J=7.0 Hz, 1H), 5.74 (t, J=6.0 Hz, 1H), 4.03 (d, J=6.5 Hz, 1H), 3.90 (d, J=6.0 Hz, 1H), 3.64 (s, 3H), 2.19-2.10 (m, 1H), 2.07-2.00 (m, 1H), 1.96-1.91 (m, 2H), 1.89-1.76 (m, 2H), 1.62 (d, J=6.5 Hz, 6H), 1.45 (s, 3H); LCMS m/z=438.3 [M+H]⁺.

*Peak 2, Example 653: 7-isopropoxy-N-(1-methyl-2-oxo-1,2-dihydropyridin-3-yl)-2-((1R,4S)-1-methyl-2-oxabicyclo[2.2.1]heptan-4-yl)imidazo[1,2-a]pyrimidine-6-carboxamide, 28.4 mg, ¹H NMR: (500 MHz, Methanol-d4) δ ppm=9.30 (s, 1H), 8.48 (d, J=7.5 Hz, 1H), 7.53 (s, 1H), 7.29 (d, J=7.0 Hz, 1H), 6.30 (t, J=7.0 Hz, 1H), 5.71 (t, J=6.0 Hz, 1H), 4.00 (d, J=6.5 Hz, 1H), 3.88 (d, J=6.0 Hz, 1H), 3.62 (s, 3H), 2.12-2.08 (m, 1H), 2.07-1.99 (m, 1H), 1.95-1.89 (m, 2H), 1.86-1.76 (m, 2H), 1.60 (d, J=6.5 Hz, 6H), 1.45 (s, 3H); LCMS m/z=438.3 [M+H]⁺.

### Example 654: 7-isopropoxy-N-(1-methyl-2-oxo-1,2-dihydropyridin-3-yl)-2-(1-methyl-2-oxabicyclo[2.2.2]octan-4-yl)imidazo[1,2-a]pyrimidine-6-carboxamide

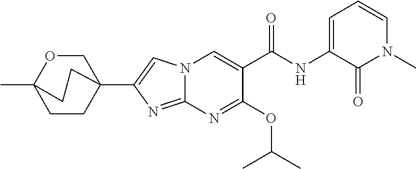

To a solution of 7-isopropoxy-2-(1-methyl-2-oxabicyclo[2.2.2]octan-4-yl)imidazo[1,2-a]pyrimidine-6-carboxylic acid (Preparation 359, 50 mg, 0.145 mmol) and 3-amino-1-methylpyridin-2(1H)-one (35.9 mg, 0.290 mmol) in T3P® (50 wt. % in EtOAc)® (2.5 mL, 50% in EtOAc) was added Pyridine (2.50 mL, 31 mmol). The mixture was stirred at rt for 16 h. To the mixture was added saturated NaHCO₃aq. to pH=7. The reaction mixture was extracted with EtOAc (50 mL×3). Then the combined organic layers were dried (Na₂SO₄) and concentrated in vacuo and the residue was purified by prep-HPLC (Welch Xtimate C18 150×25 mm×5 um; Mobile Phase: Eluting with 33%-63% water (10 mM NH₄HCO₃)-ACN) to give 7-isopropoxy-N-(1-methyl-2-oxo-1,2-dihydropyridin-3-yl)-2-(1-methyl-2-oxabicyclo[2.2.2]octan-4-yl)imidazo[1,2-a]pyrimidine-6-carboxamide (32.0 mg, 44.7% yield, 91% purity) as a white solid. ¹H NMR: (500 MHz, CDCl₃) δ: 10.81 (s, 1H), 9.14 (s, 1H), 8.56 (d, J=8.5 Hz, 1H), 7.15 (s, 1H), 7.05 (d, J=5.5 Hz, 1H), 6.26 (t, J=7.0 Hz, 1H), 5.91-5.85 (m, 1H), 4.12 (s, 2H), 3.66 (s, 3H), 2.23-2.21 (m, 2H), 2.01-1.95 (m, 4H), 1.76-1.74 (m, 2H), 1.62 (d, J=6.0 Hz, 6H), 1.18 (s, 3H); LCMS m/z=452.1 [M+H]⁺.

### Examples 655 and 656: (R)-7-(1-cyclopropylethoxy)-N-(6-(difluoromethyl)pyridin-2-yl)-2-(1-methyl-2-oxabicyclo[2.1.1]hexan-4-yl)imidazo[1,2-a]pyridine-6-carboxamide and (S)-7-(1-cyclopropylethoxy)-N-(6-(difluoromethyl)pyridin-2-yl)-2-(1-methyl-2-oxabicyclo[2.1.1]hexan-4-yl)imidazo[1,2-a]pyridine-6-carboxamide

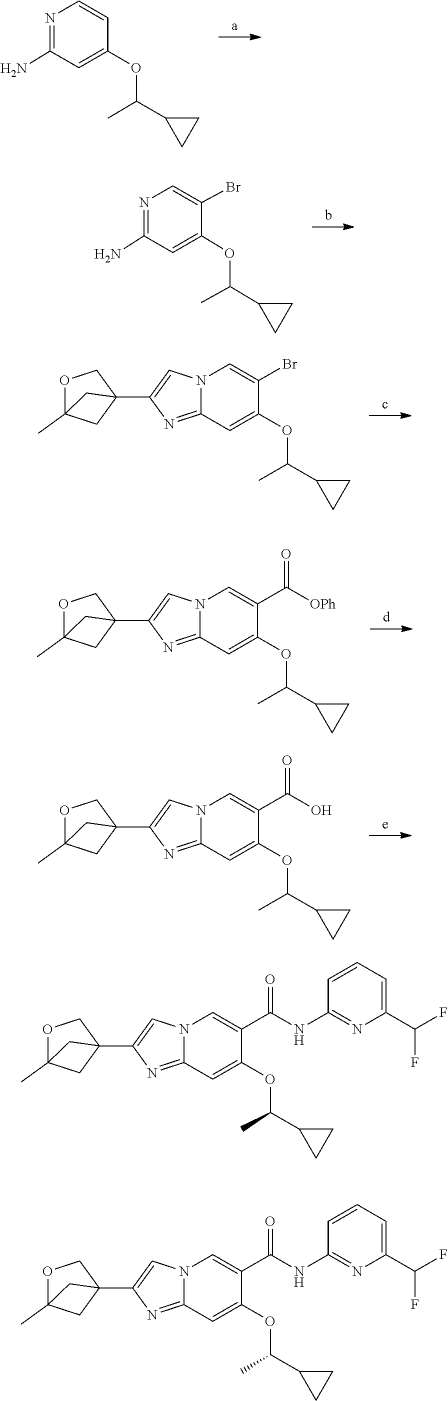

*Stereochemistry arbitrarily assigned

Step a: 1-Bromopyrrolidine-2,5-dione (0.6 g, 3.37 mmol) was added to a mixture of 4-(1-cyclopropylethoxy)pyridin-2-amine (600 mg, 3.37 mmol) in MeCN (5.6 mL) at 0° C. The reaction mixture was then stirred at room temperature for 2 h, quenched with aq. sat. NaHCO₃, extracted three times with EtOAc, washed with brine, dried over MgSO₄, filtered, concentrated, to obtain 5-bromo-4-(1-cyclopropylethoxy)pyridin-2-amine (0.7 g, 2.72 mmol, 81% yield), which was used without further purification in the next reaction. LCMS (ESI) m/z 257.0 (M+H)+.

Step b: A mixture of methyl 5-bromo-4-(1-cyclopropylethoxy)pyridin-2-amine (700 mg, 2.72 mmol), 2-bromo-1-(1-methyl-2-oxabicyclo[2.1.1]hexan-4-yl)ethanone (595 mg, 2.72 mmol) and NaHCO₃ (686 mg, 8.16 mmol) in MeCN (3.9 mL) and toluene (3.9 mL) was heated at 90° C. for 16 h. After the addition of silica and MeOH, the mixture was concentrated and purified by silica gel column chromatography (dry load, 0-40% gradient of 3:1 EtOAC/EtOH in Heptanes) to obtain methyl-bromo-7-(1-cyclopropylethoxy)-2-(1-methyl-2-oxabicyclo[2.1.1]hexan-4-yl)imidazo[1,2-a]pyridine (700 mg, 68% yield). LCMS (ESI) m/z 379.1 (M+H)⁺.

Step c: Triethylamine (4.65 mmol, 644 uL) was added to a mixture of -bromo-7-(1-cyclopropylethoxy)-2-(1-methyl-2-oxabicyclo[2.1.1]hexan-4-yl)imidazo[1,2-a]pyridine (700 mg, 1.86 mmol), diacetoxy palladium (12.5 mg, 55.8 μmol), (5-diphenylphosphanyl-9,9-dimethyl-xanthen-4-yl)-diphenyl-phosphane (64.5 mg, 111.6 μmol) and phenyl formate (4.65 mmol, 500 μL) in MeCN (4.6 mL) at room temperature. The reaction mixture was heated at 80° C. for 16 h and then purified by silica gel column chromatography (0-60% gradient of 3:1 EtOAC/EtOH in Heptanes) to obtain phenyl 7-(1-cyclopropylethoxy)-2-(1-methyl-2-oxabicyclo[2.1.1]hexan-4-yl)imidazo[1,2-a]pyridine-6-carboxylate (700 mg, 90% yield). LCMS (ESI) m/z 419.3.

Step d: A mixture of phenyl 7-(1-cyclopropylethoxy)-2-(1-methyl-2-oxabicyclo[2.1.1]hexan-4-yl)imidazo[1,2-a]pyridine-6-carboxylate (450.0 mg, 1.08 mmol) and lithium hydroxide hydrate (90.6 mg, 2.16 mmol) in MeOH (0.5 mL), THF (3.9 mL) and water (0.9 mL) was stirred 16 h at room temperature before being diluted with water and adjusted to pH ˜2 with a 4.0 M hydrochloric acid solution in dioxane. The aq. layer was then extracted three times with EtOAc, dried over MgSO₄, filtered, and concentrated to obtain 7-(1-cyclopropylethoxy)-2-(1-methyl-2-oxabicyclo[2.1.1]hexan-4-yl)imidazo[1,2-a]pyridine-6-carboxylic acid, which was used without further purification in next reaction. Assumed 100% yield. LCMS (ESI) m/z 262.2 (M+H)+.

Step e: To a mixture of 7-(1-cyclopropylethoxy)-2-(1-methyl-2-oxabicyclo[2.1.1]hexan-4-yl)imidazo[1,2-a]pyridine-6-carboxylic acid (120 mg, 350 μmol), 6-(difluoromethyl)pyridin-2-amine hydrochloride (82 mg, 455.6 μmol) in pyridine (1.2 mL) was added a solution of T3P® (50 wt. % in EtOAc)® (1.75 mmol, 1 mL) at room temperature. After stirring for 2 h, the reaction mixture was diluted with water, extracted three times with EtOAc, washed with brine, dried MgSO₄, filtered, and concentrated. The crude material was purified by mass-directed reverse-phase HPLC [XSelect CSH Prep C18 Sum OBD 19×100 mm]eluting with Mobile phase A: MeCN; Mobile phase B: H₂O, Modifier: 0.1% NH₄OH to provide 7-(1-cyclopropylethoxy)-N-(6-(difluoromethyl)pyridin-2-yl)-2-(1-methyl-2-oxabicyclo[2.1.1]hexan-4-yl)imidazo[1,2-a]pyridine-6-carboxamide (25 mg) which was submitted to chiral separation (CHIRALPAK AD-H 30×250 mm, 5 um Method: 30% MeOH w/0.1% DEA in CO₂ (flow rate: 100 mL/min, ABPR 120bar, MBPR 40 psi, column temp 40° C.) to afford:

*Peak 1, Example 655: (R)-7-(1-cyclopropylethoxy)-N-(6-(difluoromethyl)pyridin-2-yl)-2-(1-methyl-2-oxabicyclo[2.1.1]hexan-4-yl)imidazo[1,2-a]pyridine-6-carboxamide (6.1 mg), m/z 469.2 (M+H)+, 1H NMR (400 MHz, CHLOROFORM-d) δ ppm 0.39-0.58 (m, 2H) 0.74 (d, J=8.03 Hz, 2H) 1.38-1.48 (m, 1H) 1.53 (s, 3H) 1.56 (d, J=6.02 Hz, 3H) 1.88-2.00 (m, 2H) 2.06 (d, J=4.52 Hz, 2H) 4.06 (s, 2H) 4.11-4.24 (m, 1H) 6.29-6.65 (m, 1H) 6.97 (s, 1H) 7.35-7.42 (m, 2H) 7.88 (t, J=8.03 Hz, 1H) 8.47 (d, J=8.28 Hz, 1H) 9.02 (s, 1H) 10.84 (s, 1H); ¹⁹F NMR (376 MHz, CHLOROFORM-d) 6 ppm-116.32 (d, J=9.54 Hz, 1 F).

*Peak 2: Example 656: (S)-7-(1-cyclopropylethoxy)-N-(6-(difluoromethyl)pyridin-2-yl)-2-(1-methyl-2-oxabicyclo[2.1.1]hexan-4-yl)imidazo[1,2-a]pyridine-6-carboxamide (6.2 mg). m/z 469.2 (M+H)+, 1H NMR (400 MHz, CHLOROFORM-d) δ ppm 0.36-0.55 (m, 2H) 0.74 (d, J=8.28 Hz, 2H) 1.37-1.46 (m, 1H) 1.53 (s, 3H) 1.56 (d, J=6.02 Hz, 3H) 1.95 (d, J=4.77 Hz, 2H) 2.06 (br d, J=4.77 Hz, 2H) 4.06 (s, 2H) 4.17 (quin, J=6.53 Hz, 1H) 6.31-6.69 (m, 1H) 6.96 (s, 1H) 7.35-7.47 (m, 2H) 7.88 (t, J=7.91 Hz, 1H) 8.47 (d, J=8.28 Hz, 1H) 9.02 (s, 1H) 10.84 (s, 1H); ¹⁹F NMR (376 MHz, CHLOROFORM-d) 6 ppm-116.32 (d, J=9.54 Hz, 1 F).

### Examples 657 and 658: (R)-7-(1-cyclopropylethoxy)-2-(1-methyl-2-oxabicyclo[2.1.1]hexan-4-yl)-N-(6-methylpyrazolo[1,5-a]pyrimidin-3-yl)imidazo[1,2-a]pyridine-6-carboxamide and (S)-7-(1-cyclopropylethoxy)-2-(1-methyl-2-oxabicyclo[2.1.1]hexan-4-yl)-N-(6-methylpyrazolo[1,5-a]pyrimidin-3-yl)imidazo[1,2-a]pyridine-6-carboxamide

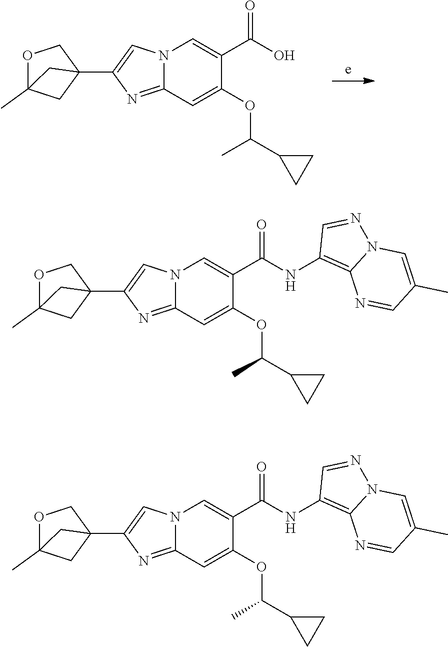

*Stereochemistry arbitrarily assigned

To a mixture of 7-(1-cyclopropylethoxy)-2-(1-methyl-2-oxabicyclo[2.1.1]hexan-4-yl)imidazo[1,2-a]pyridine-6-carboxylic acid (Example 655, 120 mg, 350.5 μmol), 6-(difluoromethyl)pyridin-2-amine hydrochloride (82 mg, 455.6 μmol) in pyridine (1.2 mL) was added a solution of T3P® (50 wt. % in EtOAc)® (1.75 mmol, 1 mL) at room temperature. After stirring for 2 h, the reaction mixture was diluted with water, extracted three times with EtOAc, washed with brine, dried MgSO₄, filtered, and concentrated. The crude material was purified by mass-directed reverse-phase HPLC [XSelect CSH Prep C18 5 um OBD 19×100 mm]eluting with Mobile phase A: MeCN; Mobile phase B: H₂O, Modifier: 0.1% NH₄OH to provide 7-(1-cyclopropylethoxy)-2-(1-methyl-2-oxabicyclo[2.1.1]hexan-4-yl)-N-(6-methylpyrazolo[1,5-a]pyrimidin-3-yl)imidazo[1,2-a]pyridine-6-carboxamide (15 mg) which was submitted to chiral separation (CHIRALPAK AD-H 30×250 mm, Sum Method: 30% EtOH w/0.1% DEA in CO2 (flow rate: 100 mL/min, ABPR 120bar, MBPR 40 psi, column temp 40° C.) to afford:

*Peak 1: Example 657, (R)-7-(1-cyclopropylethoxy)-2-(1-methyl-2-oxabicyclo[2.1.1]hexan-4-yl)-N-(6-methylpyrazolo[1,5-a]pyrimidin-3-yl)imidazo[1,2-a]pyridine-6-carboxamide (2.9 mg), m/z 473.6 (M+H)+, 1H NMR (400 MHz, CHLOROFORM-d) δ ppm 0.41-0.56 (m, 2H) 0.71 (br d, J=8.28 Hz, 2H) 1.54 (s, 3H) 1.55-1.60 (m, 1H) 1.64 (d, J=6.02 Hz, 3H) 1.97 (br d, J=4.52 Hz, 2 H) 2.08 (br d, J=4.27 Hz, 2 H) 2.39 (s, 3 H) 4.08 (s, 2 H) 4.11-4.18 (m, 1H) 6.99 (s, 1H) 7.38 (s, 1H) 8.26 (d, J=1.76 Hz, 1H) 8.41 (s, 1H) 8.86 (s, 1H) 9.08 (s, 1H) 10.66 (s, 1H).

*Peak 2: Example 658, (S)-7-(1-cyclopropylethoxy)-2-(1-methyl-2-oxabicyclo[2.1.1]hexan-4-yl)-N-(6-methylpyrazolo[1,5-a]pyrimidin-3-yl)imidazo[1,2-a]pyridine-6-carboxamide (2.9 mg), m/z 473.6 (M+H)+, 1H NMR (400 MHz, CHLOROFORM-d) δ ppm 0.41-0.53 (m, 2H) 0.70 (br d, J=8.28 Hz, 2H) 1.54 (s, 3H) 1.56-1.61 (m, 1H) 1.64 (d, J=6.02 Hz, 3H) 1.97 (br d, J=5.77 Hz, 2H) 2.08 (br d, J=4.27 Hz, 2H) 2.39 (s, 3H) 4.08 (s, 2H) 4.11-4.20 (m, 1H) 6.98 (s, 1H) 7.38 (s, 1H) 8.26 (d, J=1.76 Hz, 1H) 8.41 (s, 1H) 8.86 (s, 1H) 9.08 (s, 1H) 10.66 (s, 1H).

### Assays

Compounds of the invention were assessed for their ability to inhibit IRAK4 activity. The inhibitory properties of the compounds of the invention described herein can be evidenced by testing in any one of the following assays.

### Biochemical Assay

The 2-hour 10 μM ATP Biochemical Assay employs a MesoScale Detection (MSD) format. The kinase reaction is based on the IRAK4 phosphorylation of a biotin labeled peptide (IRAK1 activation loop sequence 360-389).

The kinase reaction in 30 μl is carried out in wells of a 384 well polypropylene assay plate, with 0.1 nM IRAK4, 1.6 μM of biotinylated peptide substrate and 10 μM ATP in 50 mM Hepes, pH 7.5, 60 mM NaCl, 5 mM MgCl₂, 0.25 mM MnCl₂, 2 mM DTT, 0.01% BSA, and 1% DMSO (from compound DMSO stocks), for 2 hour at room temperature. The activity is quenched with 11 μl of 70 mM EDTA, pH 8.

To detect the phosphorylated biotinylated peptide substrate, 30 μl of the quenched reaction mixture is added to equivalent wells of a 384 well streptavidin coated MesoScale plate (Meso Scale Discovery #L21SA-1). After a 1 hour incubation of the plate for 1 hour at room temperature with gentle mixing, the plate wells are washed 3 times with 50 mM Tris, pH 7.5, 150 mM NaCl, 0.02% Tween-20.

A 25 μl volume of 1:500 anti-P-Threonine Rabbit polyclonal Antibody plus 1:500 Goat-anti-Rabbit Sulfo Tag Antibody (Meso Scale Discovery R32AB-1) in 50 mM Tris, pH 7.5, 150 mM NaCl, 0.02% Tween-20 plus 2% BSA is then added to each well. After a 1-hour incubation of the plate for 1 hour at room temperature with gentle mixing, the plate wells are washed, 3 times with 50 mM Tris, pH 7.5, 150 mM NaCl, 0.02% Tween-20. A 40 μl volume of 2× MSD Read Buffer (Meso Scale Discovery R92TC-1) is added to each well, and the plate is read immediately in an MSD Plate Reader (Meso Scale Discovery).

The 2-hour 1 mM ATP IRAK4 Biochemical assay was performed as described above, but with 100 pM IRAK4 and 1 mM ATP.

Potency Data Table:

| Example | IRAK4 Biochemical Assay | IRAK4 Biochemical Assay |
|---|---|---|
| Number | (10 μM ATP, 2 h) IC₅₀ (μM) | (1 mM ATP, 2 h) IC₅₀ (μM) |
| 1 | | 0.0298 |
| 2 | | 0.0586 |
| 3 | | 0.2169 |
| 4 | | 0.2349 |
| 5 | | 0.0721 |
| 6 | | 0.1514 |
| 7 | | 0.0166 |
| 8 | 0.009 | 0.0263 |
| 9 | 0.009 | 0.0242 |
| 10 | 0.008 | 0.0293 |
| 11 | 0.002 | 0.0034 |
| 12 | 0.032 | |
| 13 | 0.017 | |
| 14 | 0.026 | |
| 15 | 0.031 | |
| 16 | 0.008 | |
| 17 | 0.002 | |
| 18 | 0.001 | |
| 19 | 0.006 | |
| 20 | 0.003 | |
| 21 | 0.003 | 0.0085 |
| 22 | 0.005 | 0.0180 |
| 23 | 0.025 | |
| 24 | 0.007 | 0.0210 |
| 25 | | 0.0031 |
| 26 | | 0.1238 |
| 27 | | 0.0198 |
| 28 | | 0.0969 |
| 29 | | 0.0041 |
| 30 | | 0.0735 |
| 31 | | 0.0150 |
| 32 | 0.008 | 0.0130 |
| 33 | 0.005 | 0.0140 |
| 34 | 0.057 | |
| 35 | 0.012 | |
| 36 | 0.008 | 0.0262 |
| 37 | 0.009 | 0.0253 |
| 38 | 0.002 | 0.0048 |
| 39 | 0.024 | |
| 40 | 0.009 | 0.0305 |
| 41 | 0.005 | 0.0157 |
| 42 | 0.031 | |
| 43 | 0.002 | 0.0032 |
| 44 | 0.003 | 0.0088 |
| 45 | 0.013 | 0.0401 |
| 46 | 0.037 | |
| 47 | 0.092 | |
| 48 | 0.013 | 0.0400 |
| 49 | 0.350 | |
| 50 | 0.003 | |
| 51 | 0.003 | |
| 52 | | 0.0075 |
| 53 | | 0.0352 |
| 54 | | 0.0065 |
| 55 | | 0.0135 |
| 57 | | 0.0041 |
| 58 | | 0.0105 |
| 59 | | 0.0137 |
| 60 | | 0.0288 |
| 61 | | 0.0351 |
| 62 | | 0.0038 |
| 63 | | 0.0078 |
| 64 | 0.001 | 0.0017 |
| 65 | 0.001 | 0.0015 |
| 66 | | 0.0250 |
| 67 | | 0.0397 |
| 68 | | 10.0000 |
| 69 | | 0.0444 |
| 70 | | 0.0169 |
| 71 | | 0.0311 |
| 72 | 0.008 | |
| 73 | 0.001 | |
| 74 | 0.005 | 0.0134 |
| 75 | 0.006 | |
| 76 | | 0.0095 |
| 77 | | 0.0740 |
| 78 | | 0.0475 |
| 79 | | 0.0126 |
| 80 | | 0.0290 |
| 81 | | 0.2316 |
| 82 | | 0.1191 |
| 83 | | 0.2635 |
| 84 | | 7.5106 |
| 85 | 0.001 | |
| 86 | | 0.0340 |
| 87 | | 0.0329 |
| 88 | | 0.0509 |
| 89 | | 5.6945 |
| 90 | | 0.0015 |
| 91 | 0.079 | |
| 92 | | 0.0003 |
| 93 | | 0.0002 |
| 94 | | 0.0178 |
| 95 | | 0.0005 |
| 96 | | 0.0002 |
| 97 | | 0.0003 |
| 98 | | 0.0038 |
| 99 | | 0.0073 |
| 100 | | 0.0178 |
| 101 | | 0.0175 |
| 102 | | 0.0004 |
| 103 | | 0.0032 |
| 104 | | 0.0476 |
| 105 | 0.043 | |
| 106 | 0.080 | |
| 107 | 0.013 | 0.0400 |
| 108 | | 0.0669 |
| 109 | | 0.1145 |
| 110 | | 0.0082 |
| 111 | | 0.0198 |
| 112 | | 0.5135 |
| 113 | | 0.0357 |
| 114 | | 0.0153 |
| 115 | | 0.0061 |
| 116 | | 0.0106 |
| 117 | 0.567 | |
| 118 | 0.004 | 0.0174 |
| 119 | 0.792 | |
| 120 | 0.018 | |
| 121 | 0.003 | 0.0092 |
| 122 | 0.015 | 0.0579 |
| 123 | 0.741 | |
| 124 | 0.122 | |
| 125 | 0.004 | 0.0129 |
| 126 | 0.660 | |
| 127 | 0.200 | |
| 128 | 0.067 | |
| 129 | 0.016 | |
| 130 | 0.140 | |
| 131 | 0.025 | |
| 132 | 0.034 | |
| 133 | 0.255 | |
| 134 | 0.632 | |
| 135 | 0.014 | 0.0645 |
| 136 | 0.944 | |
| 137 | 0.132 | |
| 138 | 0.065 | |
| 139 | 0.725 | |
| 140 | 0.822 | |
| 141 | 0.008 | 0.0245 |
| 142 | 0.014 | 0.0383 |
| 143 | 0.010 | 0.0354 |
| 144 | 0.007 | 0.0251 |
| 145 | 0.013 | 0.0419 |
| 146 | 0.009 | 0.0311 |
| 147 | 0.010 | 0.0417 |
| 148 | 0.087 | |
| 149 | 0.794 | |
| 150 | 0.023 | |
| 151 | 0.046 | |
| 152 | 0.023 | |
| 153 | 0.019 | |
| 154 | 0.412 | |
| 155 | 0.230 | |
| 156 | 0.147 | |
| 157 | 0.021 | |
| 158 | 0.008 | 0.0257 |
| 159 | 0.020 | |
| 160 | 0.070 | |
| 161 | 0.346 | |
| 162 | 0.001 | 0.0016 |
| 163 | 0.688 | |
| 164 | 0.037 | |
| 165 | 0.024 | |
| 166 | 0.306 | |
| 167 | 0.073 | |
| 168 | 0.011 | 0.0378 |
| 169 | 0.012 | 0.0296 |
| 170 | 0.002 | 0.0053 |
| 171 | 0.845 | |
| 172 | 0.021 | |
| 173 | 0.004 | 0.0160 |
| 174 | 0.007 | 0.0477 |
| 175 | 0.744 | |
| 176 | 0.030 | |
| 177 | 0.861 | |
| 178 | 0.305 | |
| 179 | 0.004 | 0.0089 |
| 180 | 0.019 | |
| 181 | 0.169 | |
| 182 | | 0.5593 |
| 183 | | 0.0069 |
| 184 | | 0.2078 |
| 185 | | 0.0434 |
| 186 | | 0.3119 |
| 187 | | 0.1484 |
| 188 | | 0.3262 |
| 189 | | 0.0165 |
| 190 | | 0.1109 |
| 191 | | 0.2699 |
| 192 | | 0.0429 |
| 193 | | 0.0027 |
| 194 | | 0.6237 |
| 195 | | 0.1233 |
| 196 | | 0.0091 |
| 197 | | 0.1658 |
| 198 | | 0.0759 |
| 199 | | 0.1810 |
| 200 | | 0.0115 |
| 201 | | 0.3785 |
| 202 | | 0.0137 |
| 203 | 0.004 | 0.0096 |
| 204 | | 0.4292 |
| 205 | | 0.0341 |
| 206 | | 0.0128 |
| 207 | | 0.0147 |
| 208 | | 0.0209 |
| 209 | | 0.0057 |
| 210 | | 0.3023 |
| 211 | | 0.0116 |
| 212 | | 0.0160 |
| 213 | | 0.0607 |
| 214 | | 0.0715 |
| 215 | | 0.0013 |
| 216 | | 0.0005 |
| 217 | | 0.0017 |
| 218 | | 0.0002 |
| 219 | | 0.0069 |
| 220 | | 0.0115 |
| 221 | | 0.0006 |
| 222 | | 0.0081 |
| 223 | | 0.0022 |
| 224 | | 0.0086 |
| 225 | | 0.0052 |
| 226 | | 0.0024 |
| 227 | | 0.0003 |
| 228 | | 0.0052 |
| 229 | | 0.0393 |
| 230 | | 0.0030 |
| 231 | | 0.0071 |
| 232 | | 0.0031 |
| 233 | | 0.0025 |
| 234 | | 0.0053 |
| 235 | | 0.0035 |
| 236 | | 0.0002 |
| 237 | | 0.0003 |
| 238 | | 0.0064 |
| 239 | | 0.0008 |
| 240 | | 0.0023 |
| 241 | | 0.0015 |
| 242 | | 0.0013 |
| 243 | | 0.0002 |
| 244 | | 0.0059 |
| 245 | | 0.0003 |
| 246 | | 0.0002 |
| 247 | | 0.0002 |
| 248 | | 0.0002 |
| 249 | | 0.0007 |
| 250 | | 0.0013 |
| 251 | | 0.0153 |
| 252 | | 0.0004 |
| 253 | | 0.0004 |
| 254 | | 0.0010 |
| 255 | | 0.1579 |
| 256 | | 0.0009 |
| 257 | | 0.0003 |
| 258 | | 0.0596 |
| 259 | | 0.0005 |
| 260 | | 0.0005 |
| 261 | | 0.0047 |
| 262 | | 0.0280 |
| 263 | | 0.0002 |
| 264 | | 0.0002 |
| 265 | | 0.0067 |
| 266 | | 0.0013 |
| 267 | | 0.0020 |
| 268 | | 0.0083 |
| 269 | | 0.0012 |
| 270 | | 0.0023 |
| 271 | | 0.0073 |
| 272 | | 0.0098 |
| 273 | | 0.0047 |
| 274 | | 0.0016 |
| 275 | | 0.0007 |
| 276 | | 0.0009 |
| 277 | | 0.0061 |
| 278 | | 0.0011 |
| 279 | | 0.0038 |
| 280 | | 0.0019 |
| 281 | | 0.0264 |
| 282 | | 0.0087 |
| 283 | | 0.0025 |
| 284 | | 0.0056 |
| 285 | | 0.0060 |
| 286 | | 5.9810 |
| 287 | | 7.1366 |
| 288 | | 1.2307 |
| 289 | | 0.0002 |
| 290 | | 0.0276 |
| 291 | | 0.0125 |
| 292 | | 0.0026 |
| 293 | | 0.0028 |
| 294 | | 0.0056 |
| 295 | | 0.0097 |
| 296 | | 0.0011 |
| 297 | | 0.0048 |
| 298 | | 0.0630 |
| 299 | | 0.0005 |
| 300 | | 0.0022 |
| 301 | | 0.0066 |
| 302 | | 0.0077 |
| 303 | | 0.0014 |
| 304 | | 0.0050 |
| 305 | | 0.0005 |
| 306 | | 0.0031 |
| 307 | | 0.0005 |
| 308 | | 0.0020 |
| 309 | | 0.0299 |
| 310 | | 0.0010 |
| 311 | | 0.0014 |
| 312 | | 0.0021 |
| 313 | | 0.0036 |
| 314 | | 0.0258 |
| 315 | | 0.0045 |
| 316 | | 0.0050 |
| 317 | | 0.0220 |
| 318 | | 0.0010 |
| 319 | | 0.0016 |
| 320 | | 0.0004 |
| 321 | | 0.0003 |
| 322 | | 0.0019 |
| 323 | | 0.0007 |
| 324 | | 0.0020 |
| 325 | | 0.0086 |
| 326 | | 0.0019 |
| 327 | | 0.0372 |
| 328 | | 0.0060 |
| 329 | | 0.0012 |
| 330 | | 0.0002 |
| 331 | | 0.0020 |
| 332 | | 0.0052 |
| 333 | | 0.0052 |
| 334 | | 0.0599 |
| 335 | | 0.0006 |
| 336 | | 0.0021 |
| 337 | | 0.0004 |
| 338 | | 0.0105 |
| 339 | | 0.0008 |
| 340 | | 0.0067 |
| 341 | | 0.0009 |
| 342 | | 0.0010 |
| 343 | | 0.0048 |
| 344 | | 0.0034 |
| 345 | | 0.0022 |
| 346 | | 0.0085 |
| 347 | | 0.0006 |
| 348 | | 0.0082 |
| 349 | | 0.0053 |
| 350 | | 10.0000 |
| 351 | | 10.0000 |
| 352 | | 6.5279 |
| 353 | | 0.0025 |
| 354 | | 0.0053 |
| 355 | | 0.0016 |
| 356 | | 0.0011 |
| 357 | | 0.0003 |
| 358 | | 0.0017 |
| 359 | | 0.0009 |
| 360 | | 0.0111 |
| 361 | | 0.0037 |
| 362 | | 0.0082 |
| 363 | | 0.0086 |
| 364 | | 0.0057 |
| 365 | | 0.0128 |
| 366 | | 0.0065 |
| 367 | | 0.0079 |
| 368 | | 0.0002 |
| 369 | | 0.0008 |
| 370 | | 0.0016 |
| 371 | | 0.0003 |
| 372 | | 0.0576 |
| 373 | | 0.0006 |
| 374 | | 0.0014 |
| 375 | | 0.0067 |
| 376 | | 0.0018 |
| 377 | | 0.0003 |
| 378 | | 0.0002 |
| 379 | | 0.0010 |
| 380 | | 0.0002 |
| 381 | | 0.0004 |
| 382 | | 0.0046 |
| 383 | | 0.0001 |
| 384 | | 0.0002 |
| 385 | | 0.0004 |
| 386 | | 0.0012 |
| 387 | | 0.0009 |
| 388 | | 0.0002 |
| 389 | | 0.0032 |
| 390 | | 0.0090 |
| 391 | | 0.0034 |
| 392 | | 0.0018 |
| 393 | | 0.0001 |
| 394 | | 0.0057 |
| 395 | | 0.0057 |
| 396 | | 0.0076 |
| 397 | | 0.0134 |
| 398 | | 0.0002 |
| 399 | | 0.0030 |
| 400 | | 0.0136 |
| 401 | | 0.0002 |
| 402 | | 0.0002 |
| 403 | | 0.0008 |
| 404 | | 0.0077 |
| 406 | | 0.0003 |
| 407 | | 0.0052 |
| 408 | | 0.0007 |
| 409 | | 0.0015 |
| 410 | | 0.0002 |
| 411 | | 0.0008 |
| 412 | | 0.0012 |
| 413 | | 0.0001 |
| 414 | | 0.0033 |
| 415 | | 0.0085 |
| 416 | | 0.0006 |
| 417 | | 0.0002 |
| 418 | | 0.0021 |
| 419 | | 0.0033 |
| 420 | | 0.0003 |
| 421 | | 0.0002 |
| 422 | | 0.0005 |
| 423 | | 0.0054 |
| 424 | | 0.0007 |
| 425 | | 0.0004 |
| 426 | | 0.0003 |
| 427 | | 0.0007 |
| 428 | | 0.0016 |
| 429 | | 0.0018 |
| 430 | | 0.0027 |
| 431 | | 0.0008 |
| 432 | | 0.0006 |
| 433 | | 0.0006 |
| 434 | | 0.0005 |
| 435 | | 0.0051 |
| 436 | | 0.0008 |
| 437 | | 0.0017 |
| 438 | | 0.0015 |
| 439 | | 0.0028 |
| 440 | | 0.0043 |
| 441 | | 0.0034 |
| 442 | | 0.0002 |
| 443 | | 0.0017 |
| 444 | | 0.0042 |
| 445 | | 0.0006 |
| 446 | | 0.0046 |
| 447 | | 0.0014 |
| 448 | | 0.0013 |
| 449 | | 0.0015 |
| 450 | | 0.0003 |
| 451 | | 0.0065 |
| 452 | | 0.0008 |
| 453 | | 0.0007 |
| 454 | | 0.0024 |
| 455 | | 0.0012 |
| 456 | | 0.0071 |
| 457 | | 0.0002 |
| 458 | | 0.0019 |
| 459 | | 0.0084 |
| 460 | | 0.0099 |
| 461 | | 0.0072 |
| 462 | | 0.0032 |
| 463 | | 0.0057 |
| 464 | | 0.0081 |
| 465 | | 0.0079 |
| 466 | | 0.0057 |
| 467 | | 0.0010 |
| 468 | | 0.0070 |
| 469 | | 0.0412 |
| 470 | | 0.0015 |
| 471 | | 0.0058 |
| 472 | | 0.0064 |
| 473 | | 0.0064 |
| 474 | | 0.0007 |
| 475 | | 0.0088 |
| 476 | | 0.0088 |
| 477 | | 0.0011 |
| 478 | | 0.0055 |
| 479 | | 0.0092 |
| 480 | | 0.0075 |
| 481 | | 0.0089 |
| 482 | | 0.0067 |
| 483 | | 0.0090 |
| 484 | | 0.0073 |
| 485 | | 0.0090 |
| 486 | | 0.0075 |
| 487 | | 0.0027 |
| 488 | | 0.0081 |
| 489 | | 0.0031 |
| 490 | | 0.0065 |
| 491 | | 0.0023 |
| 492 | | 0.0016 |
| 493 | | 0.0032 |
| 494 | | 0.0039 |
| 495 | | 0.0088 |
| 496 | | 0.0047 |
| 497 | | 0.0070 |
| 498 | | 0.0043 |
| 499 | | 0.0017 |
| 500 | | 0.0021 |
| 501 | | 0.0069 |
| 502 | | 0.0004 |
| 503 | | 0.0055 |
| 504 | | 0.0018 |
| 505 | | 0.0005 |
| 506 | | 0.0038 |
| 507 | | 0.0095 |
| 508 | | 0.0023 |
| 509 | | 0.0003 |
| 510 | | 0.0010 |
| 511 | | 0.0042 |
| 512 | | 0.0090 |
| 513 | | 0.0003 |
| 514 | | 0.0005 |
| 515 | | 0.0014 |
| 516 | | 0.0015 |
| 517 | | 0.0043 |
| 518 | | 0.0040 |
| 519 | | 0.0030 |
| 520 | | 0.0001 |
| 521 | | 0.0118 |
| 522 | | 0.0005 |
| 523 | | 0.0023 |
| 524 | | 0.0111 |
| 525 | | 0.0185 |
| 526 | | 0.0003 |
| 527 | | 0.0044 |
| 528 | | 0.0052 |
| 529 | | 0.0003 |
| 530 | | 0.0056 |
| 531 | | 0.0008 |
| 532 | | 0.0029 |
| 533 | | 0.0037 |
| 534 | | 0.0135 |
| 535 | | 0.0011 |
| 536 | | 0.0016 |
| 537 | | 0.0063 |
| 538 | | 0.0003 |
| 539 | | 0.0229 |
| 540 | | 0.0008 |
| 541 | | 0.0002 |
| 542 | | 0.0001 |
| 543 | | 0.0044 |
| 544 | | 0.0002 |
| 545 | | 0.0013 |
| 546 | | 0.0086 |
| 547 | | 0.0008 |
| 548 | | 0.0082 |
| 549 | | 0.0034 |
| 550 | | 0.0020 |
| 551 | | 0.0019 |
| 552 | | 0.0097 |
| 553 | | 0.0015 |
| 554 | | 0.0008 |
| 555 | | 0.0016 |
| 556 | | 0.0005 |
| 557 | | 0.0003 |
| 558 | | 0.0011 |
| 559 | | 0.0007 |
| 560 | | 0.0012 |
| 561 | | 0.0014 |
| 562 | | 0.0007 |
| 563 | | 0.0005 |
| 564 | | 0.0003 |
| 565 | | 0.0005 |
| 566 | | 0.0009 |
| 567 | | 0.0002 |
| 568 | | 0.0002 |
| 569 | | 0.0024 |
| 570 | | 0.0004 |
| 571 | | 0.0017 |
| 572 | | 0.0004 |
| 573 | | 0.0018 |
| 574 | | 0.0002 |
| 575 | | 0.0007 |
| 576 | | 0.0002 |
| 577 | | 0.0002 |
| 578 | | 0.0002 |
| 579 | | 0.0004 |
| 580 | | 0.0017 |
| 581 | | 0.0037 |
| 582 | | 0.0006 |
| 583 | | 0.0008 |
| 584 | | 0.0154 |
| 585 | | 0.0115 |
| 586 | | 0.0020 |
| 587 | | 0.0032 |
| 588 | | 0.0015 |
| 589 | | 0.0024 |
| 590 | | 0.0001 |
| 591 | | 0.0001 |
| 592 | | 0.0034 |
| 593 | | 0.0065 |
| 594 | | 0.0056 |
| 595 | | 0.0128 |
| 596 | | 0.0063 |
| 597 | | 0.0140 |
| 598 | | 0.0041 |
| 599 | | 0.0056 |
| 600 | | 0.0012 |
| 601 | | 0.0019 |
| 602 | | 0.0051 |
| 603 | | 0.0053 |
| 604 | | 0.0003 |
| 605 | | 0.0004 |
| 606 | | 0.0023 |
| 607 | | 0.0031 |
| 608 | | 0.0001 |
| 609 | | 0.0001 |
| 610 | | 0.0008 |
| 611 | | 0.0013 |
| 612 | | 0.0004 |
| 613 | | 0.0005 |
| 614 | | 0.0002 |
| 615 | | 0.0002 |
| 616 | | 0.0002 |
| 617 | | 0.0006 |
| 618 | | 0.0006 |
| 619 | | 0.0001 |
| 620 | | 0.0006 |
| 621 | | 0.0014 |
| 622 | | 0.0040 |
| 623 | | 0.0050 |
| 624 | | 0.0002 |
| 625 | | 0.0003 |
| 626 | | 0.0006 |
| 627 | | 0.0004 |
| 628 | | 0.0001 |
| 629 | | 0.0003 |
| 630 | | 0.0003 |
| 631 | | 0.0004 |
| 632 | | 0.0018 |
| 633 | | 0.0016 |
| 634 | | 0.0023 |
| 635 | | 0.0026 |
| 636 | | 0.0037 |
| 637 | | 0.0445 |
| 638 | | 0.0045 |
| 639 | | 0.0181 |
| 640 | | 0.0007 |
| 641 | | 0.0062 |
| 642 | | 0.0013 |
| 643 | | 0.0004 |
| 644 | | 0.0012 |
| 645 | | 0.0005 |
| 646 | | 0.0008 |
| 647 | | 0.0013 |
| 648 | | 0.0045 |
| 649 | | 0.0002 |
| 650 | | 0.0001 |
| 651 | | 0.0005 |
| 652 | | 0.0005 |
| 653 | | 0.0011 |
| 654 | | 0.0005 |
| 655 | | 0.0004 |
| 656 | | 0.0002 |
| 657 | | 0.0009 |
| 658 | | 0.0006 |

1. A compound of formula (I′): 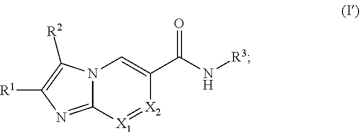
or a pharmaceutically acceptable salt thereof, wherein:
R¹ is selected from the group consisting of halo, C₁₋₅ alkyl, C₃₋₆cycloalkyl, —C₁₋₂ alkyl-C₃₋₆cycloalkyl, a fully saturated 4 to 7 membered heterocycle containing 1 to 2 heteroatoms independently selected from nitrogen, sulfur and oxygen, —C₁₋₂ alkyl-C₄₋₇ heterocycle, wherein the C₄₋₇ heterocycle may be fully or partially saturated and contains 1 to 2 heteroatoms independently selected from nitrogen, sulfur and oxygen, —C₁₋₄ alkyl-O—C₁₋₂ alkyl, a fully saturated 5 to 8 membered bridged-carbocyclic ring, a fully saturated 5 to 8 membered bridged-heterocyclic ring system having 1 to 2 heteroatoms independently selected from nitrogen and oxygen, a 5 to 10 membered fused heterobicyclic ring system having 1 to 2 heteroatoms independently selected from nitrogen and oxygen and a 5 to 10 membered spiro heterobicyclic ring system having 1 to 2 heteroatoms independently selected from nitrogen and oxygen, wherein R¹ may be optionally substituted with 1, 2 or 3 substituents R^{1a} which are independently selected from halo, nitrile, oxo, halo-substituted C₁₋₄ alkyl, hydroxy-substituted C₁₋₄ alkyl, C₁₋₄ alkyl, C₄₋₇ heterocycle containing 1 to 2 heteroatoms independently selected from nitrogen and oxygen, C₁₋₄ alkyl-O—C₁₋₂ alkyl, hydroxyl and C₁₋₄ alkoxy;
R² is hydrogen, C₁₋₄ alkyl or halogen;
R³ is selected from the group consisting of
i. a 5 or 6 membered heteroaryl having 1 to 3 heteroatoms independently selected from nitrogen, oxygen and sulfur, said heteroaryl is optionally substituted with 1 to 3 R⁴;
ii. Phenyl optionally substituted with 1 to 3 R⁴,
iii. a 5-6 membered partially or fully saturated heterocycle having 1 to 2 heteroatoms independently selected from oxygen and nitrogen, said heterocycle may be optionally substituted with 1 to 3 R⁴;
iv. a partially or fully saturated C₃₋₆ cycloalkyl which may be optionally substituted with 1 to 3 R⁴;
v. a 7 to 10 membered fused heterobicyclic ring system having 1, 2 or 3 heteroatoms independently selected from nitrogen and oxygen, said ring system is optionally substituted with 1 to 3 R⁴; and
vi. a 7 to 10 membered fused bicyclic ring system, said ring system is optionally substituted with 1 to 3 R⁴;
X₁ and X₂ are independently selected from N, CH and CR⁵, wherein only one of X₁ or X₂ may be N;
R⁵ is selected from halogen, C₁₋₄alkyl, nitrile and —OR⁶, wherein the C₁₋₄alkyl is optionally substituted with C₁₋₄alkoxy;
R⁶ is hydrogen, C₁₋₅alkyl, C₃₋₆cycloalkyl, a 4 to 7 membered partially or fully saturated heterocycle containing 1 or 2 heteroatoms selected from nitrogen and oxygen, a 5 to 10 membered spiro carbocyclic ring and a 5 to 10 membered spiro heterobicyclic ring system having 1 to 2 heteroatoms independently selected from nitrogen and oxygen, wherein the C₁₋₅alkyl represented by R⁶ is optionally substituted with 1 to 3 substituents R^{6′} independently selected from halogen, hydroxyl, C₁₋₄alkoxy, halo-substituted C₁₋₄alkoxy, C₃₋₆cycloalkyl, phenyl, a 4 to 7 membered partially or fully saturated heterocycle containing 1 or 2 heteroatoms selected from nitrogen and oxygen, an a fully saturated 5 to 8 membered bridged-heterocyclic ring system having 1 to 2 heteroatoms independently selected from nitrogen and oxygen; the C₃₋₆cycloalkyl represented by R⁶ is optionally substituted with 1 to 3 substituents R^{6b} independently selected from halo, C₁₋₄alky, halo-substituted C₁₋₄ alkyl, and C₁₋₄alkoxy; the 4 to 7 membered partially or fully saturated heterocycle, the 5 to 10 membered spiro carbocyclic ring and 5 to 10 membered spiro heterobicyclic ring system represented by R⁶ is optionally substituted with 1 to 3 substituents R^{6′} independently selected from C₁₋₄alky and oxo, and wherein said C₃₋₆cycloalkyl, phenyl, 4 to 7 membered partially or fully saturated heterocycle represented by R^{6a} are optionally substituted with 1 to 3 R⁷;
each R⁷ is independently selected from oxo, halo, halo-substituted C₁₋₄ alkyl and C₁₋₄ alkyl;
R⁴ for each occurrence, is independently selected from CN, hydroxyl, C₁₋₄ alkyl, CN-substituted C₁₋₄ alkyl, oxo, halo, halo-substituted C₁₋₄alkyl, C₁₋₄ alkoxy-C₁₋₄ alkyl, —NR⁸R⁹, C₁₋₄ alkoxy, C₁₋₄ alkoxy-C₁₋₄ alkoxy, hydroxy-substituted C₁₋₄ alkyl, halo-substituted C₁₋₄ alkoxy, C₃₋₆cycloalkyl, —C₁₋₄alkyl-C₃₋₆cycloalkyl, C(O)NR¹⁰R¹¹, a C₄₋₇ heterocycle, and a 5 or 6 membered heteroaryl having 1 to 2 heteroatoms independently selected from nitrogen, oxygen and sulfur, said C₃₋₆cycloalkyl and heteroaryl may be optionally substituted with 1 to 2 substituents independently selected from the group consisting of C₁₋₄ alkyl, hydroxyl and halogen; or two R⁴ groups on the same atom may form a C₃₋₆cycloalkyl, or two R⁴ groups on adjacent ring atoms may form phenyl, C₄₋₆ carbocycle, C₄₋₆ heterocycle, or a 7 membered bridged ring system optionally having 1 heteroatom selected from nitrogen and oxygen, wherein said phenyl, C₃₋₆cycloalkyl C₄₋₆ carbocycle and C₄₋₆ heterocycle may be optionally substituted with 1 to 2 C₁₋₄ alkyl, halo or halo-substituted C₁₋₄alkyl;
R⁸ and R⁹ are each independently selected from hydrogen, —C(O)C₁₋₄ alkyl and C₁₋₄ alkyl; or R⁸ and R⁹ may combine to form a 4 to 6 membered saturated ring optionally containing one additional heteroatom selected from nitrogen or oxygen wherein said additional nitrogen may be optionally substituted with C₁₋₄ alkyl; and
R¹⁰ and R¹¹ are each independently selected from hydrogen and C₁₋₄ alkyl.

2. The compound of claim 1, wherein the compounds is represented by formula (I): 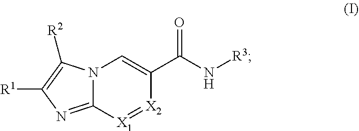
or a pharmaceutically acceptable salt thereof, wherein:
R¹ is selected from the group consisting of C₁₋₅ alkyl, C₃₋₆cycloalkyl, —C₁₋₂ alkyl-C₃₋₆cycloalkyl, a fully saturated 4 to 7 membered heterocycle containing 1 to 2 heteroatoms independently selected from nitrogen, sulfur and oxygen, —C₁₋₂ alkyl-C₄₋₇ heterocycle, wherein the C₄₋₇ heterocycle may be fully or partially saturated and contains 1 to 2 heteroatoms independently selected from nitrogen, sulfur and oxygen, —C₁₋₄ alkyl-O—C₁₋₂ alkyl, a fully saturated 5 to 8 membered bridged-carbocyclic ring, a fully saturated 5 to 8 membered bridged-heterocyclic ring system having 1 to 2 heteroatoms independently selected from nitrogen and oxygen, a 5 to 10 membered fused heterobicyclic ring system having 1 to 2 heteroatoms independently selected from nitrogen and oxygen and a 5 to 10 membered spiro heterobicyclic ring system having 1 to 2 heteroatoms independently selected from nitrogen and oxygen, wherein R¹ may be optionally substituted with 1, 2 or 3 substituents which are independently selected from halo, nitrile, oxo, halo-substituted C₁₋₄ alkyl, hydroxy-substituted C₁₋₄ alkyl, C₁₋₄ alkyl, C₄₋₇ heterocycle containing 1 to 2 heteroatoms independently selected from nitrogen and oxygen, C₁₋₄ alkyl-O—C₁₋₂ alkyl, hydroxyl and C₁₋₄ alkoxy;
R² is hydrogen, C₁₋₄ alkyl or halogen;
R³ is selected from the group consisting of
i. a 5 or 6 membered heteroaryl having 1 to 2 heteroatoms independently selected from nitrogen, oxygen and sulfur, said heteroaryl is optionally substituted with 1 to 3 R⁴;
ii. Phenyl optionally substituted with 1 to 3 R⁴,
iii. a 5-6 membered partially or fully saturated heterocycle having 1 to 2 heteroatoms independently selected from oxygen and nitrogen, said heterocycle may be optionally substituted with 1 to 3 R⁴;
iv. a partially or fully saturated C₃₋₆ cycloalkyl which may be optionally substituted with 1 to 3 R⁴;
v. a 7 to 10 membered fused heterobicyclic ring system having 1, 2 or 3 heteroatoms independently selected from nitrogen and oxygen, said ring system is optionally substituted with 1 to 3 R⁴; and
vi. a 7 to 10 membered fused bicyclic ring system, said ring system is optionally substituted with 1 to 3 R⁴;
X₁ and X₂ are independently selected from N, CH and CR⁵, wherein only one of X₁ or X₂ may be N;
R⁵ is selected from halogen, C₁₋₄alkyl, nitrile and —OR⁶;
R⁶ is hydrogen or an optionally substituted C₁₋₅alkyl having 1 to 3 substituents independently selected from halogen, hydroxyl, C₁₋₄alkoxy, C₃₋₆cycloalkyl, phenyl and a 4 to 7 membered partially or fully saturated heterocycle containing 1 or 2 heteroatoms selected from nitrogen and oxygen, wherein said C₃₋₆cycloalkyl and phenyl may be optionally substituted with 1 to 3 R⁷;
each R⁷ is independently selected from oxo, halo, halo-substituted C₁₋₄ alkyl and C₁₋₄ alkyl;
R⁴ for each occurrence, is independently selected from CN, hydroxyl, C₁₋₄ alkyl, CN-substituted C₁₋₄ alkyl, oxo, halo, halo-substituted C₁₋₄alkyl, —NR⁸R⁹, C₁₋₄ alkoxy, C₁₋₄ alkoxy-C₁₋₄ alkoxy, hydroxy-substituted C₁₋₄ alkyl, halo-substituted C₁₋₄ alkoxy, C₃₋₆cycloalkyl, C(O)NR¹⁰R¹¹ and a 5 or 6 membered heteroaryl having 1 to 2 heteroatoms independently selected from nitrogen, oxygen and sulfur, said C₃₋₆cycloalkyl and heteroaryl may be optionally substituted with 1 to 2 substituents independently selected from the group consisting of C₁₋₄ alkyl, hydroxyl and halogen; or two R⁴ groups on the same atom may form a C₃₋₆cycloalkyl, or two R⁴ groups on adjacent ring atoms may form phenyl, C₄₋₆ carbocycle, C₄₋₆ heterocycle, or a 7 membered bridged ring system optionally having 1 heteroatom selected from nitrogen and oxygen, wherein said phenyl, C₃₋₆cycloalkyl C₄₋₆ carbocycle and C₄₋₆ heterocycle may be optionally substituted with 1 to 2 C₁₋₄ alkyl, halo or halo-substituted C₁₋₄alkyl;
R⁸ and R⁹ are each independently selected from hydrogen, —C(O)C₁₋₄ alkyl and C₁₋₄ alkyl; or R⁸ and R⁹ may combine to form a 4 to 6 membered saturated ring optionally containing one additional heteroatom selected from nitrogen or oxygen wherein said additional nitrogen may be optionally substituted with C₁₋₄ alkyl; and
R¹⁰ and R¹¹ are each independently selected from hydrogen and C₁₋₄ alkyl.

3. The compound of claim 1 or 2 of formula (I): 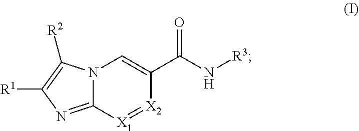
or a pharmaceutically acceptable salt thereof, wherein:
R² is H; and
X₁ is N or CH; and X² is CR⁵.

4. The compound of claim 1 or 2 of formula (I): 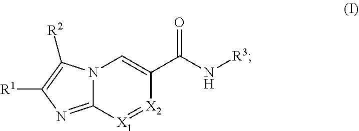
or a pharmaceutically acceptable salt thereof, wherein:
R² is H; and
X₁ is CR⁵ and X² is N or CH.

5. The compound of claim 1 or 2 of formula (Ia): 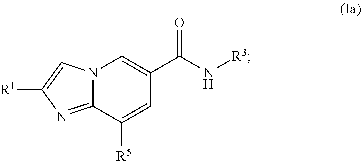
or a pharmaceutically acceptable salt thereof.

6. The compound of claim 1 or 2 of formula (Ib): 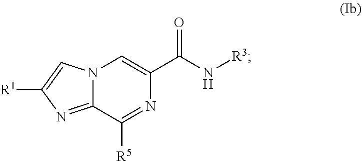
or a pharmaceutically acceptable salt thereof.

7. The compound of claim 1 or 2 of formula (Ic): 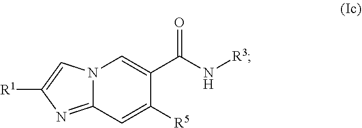
or a pharmaceutically acceptable salt thereof.

8. The compound of claim 1 or 2 of formula (Id): 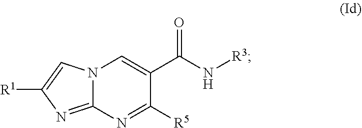
or a pharmaceutically acceptable salt thereof.

9. The compound of any one of the preceding claims, or a pharmaceutically acceptable salt thereof, wherein:
R³ is selected from the group consisting of
i. a 5 or 6 membered heteroaryl having 1 to 2 heteroatoms independently selected from nitrogen, oxygen and sulfur, said heteroaryl is optionally substituted with 1 to 3 R⁴;
ii. Phenyl optionally substituted with 1 to 3 R⁴,
iii. a 5-6 membered partially or fully saturated heterocycle having 1 to 2 heteroatoms independently selected from oxygen and nitrogen, said heterocycle may be optionally substituted with 1 to 3 R⁴;
iv. a partially or fully saturated C₃₋₆ cycloalkyl which may be optionally substituted with 1 to 3 R⁴;
v. a 7 to 10 membered fused heterobicyclic ring system having 1, 2 or 3 heteroatoms independently selected from nitrogen and oxygen, said ring system is optionally substituted with 1 to 3 R⁴; and
vi. a 7 to 10 membered fused bicyclic ring system, said ring system is optionally substituted with 1 to 3 R⁴.

10. The compound of claim 9, or a pharmaceutically acceptable salt thereof, wherein:
R³ is a 5 or 6 membered monocyclic heteroaryl having 1 to 2 heteroatoms independently selected from nitrogen and oxygen, pyridinyl-2(1H)-one or a 9 to 10 membered bicyclic heteroaryl having 1 to 3 heteroatoms independently selected from nitrogen and oxygen, wherein the monocyclic heteroaryl, pyridinyl-2(1H)-one or the bicyclic heteroaryl are each optionally substituted with 1 or 2 R⁴.

11. The compound of claim 10, or a pharmaceutically acceptable salt thereof, wherein:
R³ is a 5 or 6 membered monocyclic heteroaryl having 1 to 2 nitrogen atoms, pyridinyl-2(1H)-one or a 9 to 10 membered bicyclic heteroaryl having 2 to 3 nitrogen atoms, wherein the monocyclic heteroaryl, pyridinyl-2(1H)-one or the bicyclic heteroaryl are each optionally substituted with 1 or 2 R⁴.

12. The compound of any one of claims 1-11, or a pharmaceutically acceptable salt thereof, wherein R⁴, for each occurrence, is independently selected from hydroxyl, halo, halo-substituted C₁₋₄ alkyl, —NR⁸R⁹, and C₁₋₄ alkyl.

13. The compound of any one of claims 1 to 8, or a pharmaceutically acceptable salt thereof, wherein:
R³ is selected from pyridyl, oxazolyl, pyrazinyl, oxadiazoyl, thiophenyl, thiazolyl, isothiazolyl, pyrazolyl, imidazolyl, said R³ is optionally substituted with 1 to 2 substituents independently selected from the group consisting of halo, halo-substituted C₁₋₄ alkyl, —NR⁸R⁹, and C₁₋₄ alkyl.

14. The compound of any one of claims 1 to 8, or a pharmaceutically acceptable salt thereof, wherein:
R³ is pyridinyl-2(1H)-one optionally substituted with 1 to 2 substituents independently selected from the group consisting of halo, halo-substituted C₁₋₄ alkyl, —NR⁸R⁹, and C₁₋₄ alkyl.

15. The compound of any one of any one of claims 1 to 8, or a pharmaceutically acceptable salt thereof, wherein:
R³ is phenyl, said phenyl is optionally substituted with 1 to 2 substituents independently selected from the group consisting of halo, halo-substituted C₁₋₄ alkyl, —NR⁸R⁹, and C₁₋₄ alkyl.

16. The compound of any one of any one of claims 1 to 8, or a pharmaceutically acceptable salt thereof, wherein:
R³ is selected from the group consisting of 1,3-dihydroisobenzofuran, 2,3-dihydrobenzofuran, 4-oxaspiro[bicyclo[3.2.0]heptane-6,1′-cyclobutane], oxaspiro[bicyclo[3.2.0]heptane-6,1′-cyclobutane], bicyclo[3.1.0]hexane, cyclohexyl, spiro[2.5]octane, (1S,5R)-1-methylbicyclo[3.1.0]hexane, spiro[2.5]octane, 1,2,3,4-tetrahydronaphthalen, tetrahydrofuran, 2,3-dihydrobenzofuran, 2,3-dihydro-1H-indene, 4-methyl-3,4-dihydro-2H-benzo[b][1,4]oxazine, pyrido[3,2-d]pyrimidinyl, 1,2,3,4-tetrahydro-1,4-epoxynaphthalene, 5,6-dihydro-4H-pyrrolo[1,2-b]pyrazole, 6,7-dihydro-5H-cyclopenta[b]pyridine, 1,2,3,4-tetrahydronaphthalene, indolin-2-one, 2,3-dihydrobenzofuran, pyrazolo[1,5-a]pyrimidine, 1-methyl-2-oxo-1,2,3,4-tetrahydroquinoline, 3,4-dihydroquinolin-2(1H)-one, chromane, and isochromane, wherein said R³ is optionally substituted with 1 to 2 substituents independently selected from the group consisting halo, halo-substituted C₁₋₄ alkyl, —NR⁸R⁹, and C₁₋₄ alkyl.

17. The compound of any one of claims 1 to 4 of formula (II): 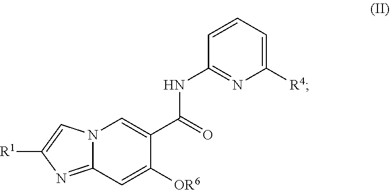
or a pharmaceutically acceptable salt thereof, wherein:
R⁶ is an optionally substituted C₁₋₅alkyl having 1 to 3 substituents independently selected from halogen, hydroxyl, C₁₋₄alkoxy, C₃₋₆cycloalkyl, phenyl and a 4 to 7 membered partially or fully saturated heterocycle containing 1 or 2 heteroatoms selected from nitrogen and oxygen, wherein said C₃₋₆cycloalkyl and phenyl may be optionally substituted with 1 to 3 R⁷.

18. The compound of any one of claims 1 to 4 of formula (III): 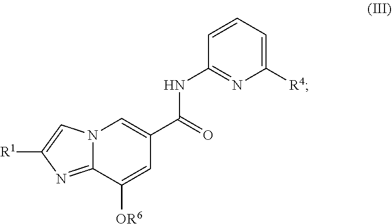
or a pharmaceutically acceptable salt thereof, wherein:
R⁶ is an optionally substituted C₁₋₅alkyl having 1 to 3 substituents independently selected from halogen, hydroxyl, C₁₋₄alkoxy, C₃₋₆cycloalkyl, phenyl and a 4 to 7 membered partially or fully saturated heterocycle containing 1 or 2 heteroatoms selected from nitrogen and oxygen, wherein said C₃₋₆cycloalkyl and phenyl may be optionally substituted with 1 to 3 R⁷.

19. The compound of any one of claims 1 to 4 of formula (IV): 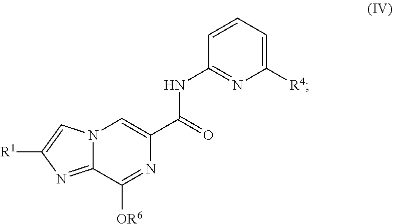
or a pharmaceutically acceptable salt thereof, wherein:
R⁶ is an optionally substituted C₁₋₅alkyl having 1 to 3 substituents independently selected from halogen, hydroxyl, C₁₋₄alkoxy, C₃₋₆cycloalkyl, phenyl and a 4 to 7 membered partially or fully saturated heterocycle containing 1 or 2 heteroatoms selected from nitrogen and oxygen, wherein said C₃₋₆cycloalkyl and phenyl may be optionally substituted with 1 to 3 R⁷.

20. The compound of any one of the preceding claims, or a pharmaceutically acceptable salt thereof, wherein:
R¹ is a fully saturated C₄₋₇ heterocycle or a 5 to 8 membered bridged-heterocyclic ring system which contain 1 to 2 heteroatoms independently selected from nitrogen and oxygen, said C₄₋₇ heterocycle or a 5 to 8 membered bridged-heterocyclic ring system may be optionally substituted with 1 or 2 substituents independently selected from the group consisting of C₁₋₄alkyl, halogen, halo-substituted C₁₋₄ alkyl, hydroxyl and C₁₋₄alkoxy; or R¹ is a C₁₋₅ alkyl which is optionally substituted with 1 or 3 substituents independently selected from the group consisting of halogen, halo-substituted C₁₋₄ alkyl, hydroxy-substituted C₁₋₄ alkyl, hydroxyl, C₁₋₄alkoxy and C₃₋₆cycloalkyl, wherein said C₃₋₆cycloalkyl is optionally substituted with 1 or 2 substituents independently selected from the group consisting of halogen, halo-substituted C₁₋₄ alkyl, hydroxyl and C₁₋₄alkoxy.

21. The compound of any one of the preceding claims, or a pharmaceutically acceptable salt thereof, wherein:
R¹ is a fully saturated C₄₋₇ heterocycle or a 5 to 8 membered bridged-heterocyclic ring system which contain 1 to 2 heteroatoms independently selected from nitrogen and oxygen, said C₄₋₇ heterocycle or a 5 to 8 membered bridged-heterocyclic ring system may be optionally substituted with 1 or 2 substituents independently selected from the group consisting of C₁₋₄alkyl, halogen, halo-substituted C₁₋₄ alkyl, hydroxyl and C₁₋₄alkoxy.

22. The compound of any one of claims 1-19, or a pharmaceutically acceptable salt thereof, wherein:
R¹ is a C₁₋₅ alkyl which is optionally substituted with 1 or 3 substituents independently selected from the group consisting of halogen, halo-substituted C₁₋₄ alkyl, hydroxyl, C₁₋₄alkoxy and C₃₋₆cycloalkyl, wherein said C₃₋₆cycloalkyl is optionally substituted with 1 or 2 substituents independently selected from the group consisting of halogen, halo-substituted C₁₋₄ alkyl, hydroxyl and C₁₋₄alkoxy.

23. The compound of any one of claims 1-19, or a pharmaceutically acceptable salt thereof, wherein:
R¹ is a C₁₋₅ alkyl substituted with 1 or 3 substituents independently selected from the group consisting of halo-substituted C₁₋₄ alkyl, hydroxyl, C₁₋₄alkoxy and C₃₋₆cycloalkyl, wherein said C₃₋₆cycloalkyl is optionally substituted with 1 or 2 substituents independently selected from the group consisting of halogen, halo-substituted C₁₋₄ alkyl, hydroxyl and C₁₋₄alkoxy.

24. The compound of any one of claims 1-19, or a pharmaceutically acceptable salt thereof, wherein:
R¹ is selected from the group consisting of C₃₋₆cycloalkyl, —C₁₋₂ alkyl-C₃₋₆cycloalkyl, a fully saturated 4 to 7 membered heterocycle containing 1 to 2 heteroatoms independently selected from nitrogen, sulfur and oxygen, —C₁₋₂ alkyl-C₄₋₇ heterocycle, wherein the C₄₋₇ heterocycle may be fully or partially saturated and contains 1 to 2 heteroatoms independently selected from nitrogen, sulfur and oxygen, a fully saturated 5 to 8 membered bridged-carbocyclic ring, a fully saturated 5 to 8 membered bridged-heterocyclic ring system having 1 to 2 heteroatoms independently selected from nitrogen and oxygen, a 5 to 10 membered fused heterobicyclic ring system having 1 to 2 heteroatoms independently selected from nitrogen and oxygen and a 5 to 10 membered spiro heterobicyclic ring system having 1 to 2 heteroatoms independently selected from nitrogen and oxygen, wherein R¹ may be optionally substituted with 1, 2 or 3 substituents R^{1a} which are independently selected from halo, nitrile, oxo, halo-substituted C₁₋₄ alkyl, hydroxy-substituted C₁₋₄ alkyl, C₁₋₄ alkyl, C₄₋₇ heterocycle containing 1 to 2 heteroatoms independently selected from nitrogen and oxygen, C₁₋₄ alkyl-O—C₁₋₂ alkyl, hydroxyl and C₁₋₄ alkoxy.

25. The compound of any one of claims 1-19, or a pharmaceutically acceptable salt thereof, wherein R¹ is a 5 to 8 membered bridged-heterocyclic ring system which contains 1 to 2 heteroatoms independently selected from nitrogen and oxygen, wherein the 5 to 8 membered bridged-heterocyclic ring system is optionally substituted with one or two substituents R^{1a} independently selected from C₁₋₄alkyl, halogen, halo-substituted C₁₋₄ alkyl, hydroxyl and C₁₋₄alkoxy.

26. The compound of claim 25, or a pharmaceutically acceptable salt thereof, wherein R¹ is a 5 to 8 membered bridged-heterocyclic ring system containing one oxygen atom and wherein the 5 to 8 membered bridged-heterocyclic ring system is optionally substituted with one or two substituents R^{1a} independently selected from C₁₋₄alkyl, halogen, halo-substituted C₁₋₄ alkyl, hydroxyl and C₁₋₄alkoxy.

27. The compound of claim 25, or a pharmaceutically acceptable salt thereof, wherein R¹ is a 5 to 8 membered bridged-heterocyclic ring system represented by the following formula: 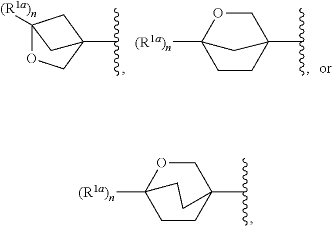
wherein R^{1a} is C₁₋₄ alkyl or halo-substituted C₁₋₄ alkyl; and n is 0 or 1.

28. The compound of claim 27, or a pharmaceutically acceptable salt thereof, wherein R^{1a} is CH₃ or CH₂F.

29. The compound of any one of claims 1 to 4, or a pharmaceutically acceptable salt thereof, wherein:
R¹ is a fully saturated C₄₋₇ heterocycle or a 5 to 8 membered bridged-heterocyclic ring system which contain 1 to 2 heteroatoms independently selected from nitrogen and oxygen, said C₄₋₇ heterocycle or a 5 to 8 membered bridged-heterocyclic ring system may be optionally substituted with 1 or 2 substituents independently selected from the group consisting of C₁₋₄alkyl, halogen, halo-substituted C₁₋₄ alkyl, hydroxyl and C₁₋₄alkoxy; and
R³ is pyridinyl substituted with 1 or 2 substituents independently selected from and C₁₋₄ alkyl and halo-substituted C₁₋₄ alkyl.

30. The compound of any one of claims 1-16 and 20-29, wherein R⁶ is an optionally substituted C₁₋₅alkyl or an optionally substituted C₃₋₆cycloalkyl, wherein the C₁₋₅alkyl is optionally substituted with 1 to 3 substituents independently selected from halogen, hydroxyl and C₁₋₄alkoxy and the C₃₋₆cycloalkyl is optionally substituted with 1 to 3 substituents independently selected from halo, C₁₋₄alky, halo-substituted C₁₋₄ alkyl and C₁₋₄alkoxy.

31. The compound of claim 1, wherein the compound is represented by the following formula: 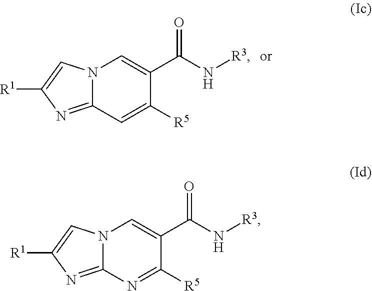
or a pharmaceutically acceptable salt thereof, wherein:
R¹ is a 5 to 8 membered bridged-heterocyclic ring system which contains 1 to 2 heteroatoms independently selected from nitrogen and oxygen, wherein the 5 to 8 membered bridged-heterocyclic ring system is optionally substituted with one or two substituents R^{1a};
R^{1a}, for each occurrence, is independently selected from C₁₋₄alkyl, halogen, halo-substituted C₁₋₄ alkyl, hydroxyl and C₁₋₄alkoxy;
R³ is a 5 or 6 membered monocyclic heteroaryl having 1 to 2 heteroatoms independently selected from nitrogen and oxygen, pyridinyl-2(1H)-one or a 9 to 10 membered bicyclic heteroaryl having 1 to 3 heteroatoms independently selected from nitrogen and oxygen, wherein the monocyclic heteroaryl, pyridinyl-2(1H)-one or the bicyclic heteroaryl are each optionally substituted with 1 or 2 R⁴;
R⁴, for each occurrence, is independently selected from hydroxyl, halo, halo-substituted C₁₋₄ alkyl, —NR⁸R⁹, and C₁₋₄ alkyl;
R⁵ is OR⁶; and
R⁶ is an optionally substituted C₁₋₅alkyl or an optionally substituted C₃₋₆cycloalkyl, wherein the C₁₋₅alkyl is optionally substituted with 1 to 3 substituents independently selected from halogen, hydroxyl and C₁₋₄alkoxy and the C₃₋₆cycloalkyl is optionally substituted with 1 to 3 substituents independently selected from halo, C₁₋₄alky, halo-substituted C₁₋₄ alkyl and C₁₋₄alkoxy.

32. The compound of claim 31, or a pharmaceutically acceptable salt thereof, wherein
R¹ is a 5 to 8 membered bridged-heterocyclic ring system containing one oxygen atom, wherein the 5 to 8 membered bridged-heterocyclic ring system is optionally substituted with one substituent R^{1a};
R^{1a} is C₁₋₄alkyl or halo-substituted C₁₋₄ alkyl;
R³ is a 5 or 6 membered monocyclic heteroaryl having 1 to 2 nitrogen atoms, pyridinyl-2(1H)-one or a 9 to 10 membered bicyclic heteroaryl having 2 to 3 nitrogen atoms, wherein the monocyclic heteroaryl, pyridinyl-2(1H)-one or the bicyclic heteroaryl are each optionally substituted with 1 or 2 R⁴;
R⁴, for each occurrence, is independently selected from hydroxyl, halo-substituted C₁₋₄ alkyl, and C₁₋₄ alkyl;
R⁵ is OR⁶; and
R⁶ is an optionally substituted C₁₋₅alkyl or an optionally substituted C₃₋₆cycloalkyl, wherein the C₁₋₅alkyl is optionally substituted with 1 to 3 substituents independently selected from halogen and the C₃₋₆cycloalkyl is optionally substituted with 1 to 3 substituents independently selected from C₁₋₄alkyl, halo-substituted C₁₋₄alkyl and halogen.

33. The compound of claim 32, or a pharmaceutically acceptable salt thereof, wherein:
R¹ is 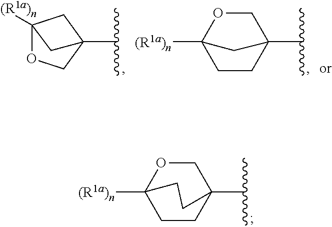
R^{1a} is C₁₋₄ alkyl or halo-substituted C₁₋₄ alkyl;
n is 0 or 1;
R³ is 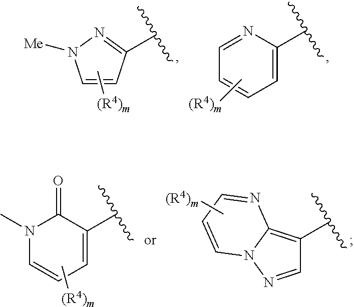
R⁴ is hydroxyl, C₁₋₄ alkyl or halo-substituted C₁₋₄ alkyl;
m is 0, 1 or 2;
R⁵ is OR⁶; and
R⁶ is C₁₋₄alkyl or C₄₋₆cycloalkyl.

34. The compound of claim 33, or a pharmaceutically acceptable salt thereof, wherein R^{1a} is CH₃ or CH₂F; and R⁴ is CH₃, CHF₂ or OH, and R⁶ is —CH(CH₃)₂, cyclobutyl, or cyclopentyl.

35. The compound of formula I of claim 1, selected from a compound of any one of Examples 1-658 or a pharmaceutically acceptable salt thereof.

36. A pharmaceutical composition comprising a compound of any one of the preceding claims or a pharmaceutically acceptable salt thereof.

37. The pharmaceutical composition of claim 36, further comprising one or more additional pharmaceutical agent(s).

38. A method of treating an IRAK4 mediated disease in a subject comprising administering to the subject a compound or a pharmaceutically acceptable salt thereof of any one of claims 1 to 35 or a pharmaceutical composition of any one of claims 36 to 37.

39. The method of claim 38, wherein the IRAK4 mediated disease is selected from the group consisting from Rheumatoid Arthritis, Psoriatic arthritis, Osteoarthritis, Systemic Lupus Erythematosus, Lupus nephritis, Ankylosing Spondylitis, Osteoporosis, Systemic sclerosis, Multiple Sclerosis, Psoriasis, Type I diabetes, Type II diabetes, Inflammatory Bowel Disease, Crohn's Disease, Ulcerative Colitis, Hyperimmunoglobulinaemia D, periodic fever syndrome, Cryopyrin-associated periodic syndromes, Schnitzler's syndrome, Systemic juvenile idiopathic arthritis, Adult's onset Still's disease, Gout, Pseudogout, SAPHO syndrome, Castleman's disease, Sepsis, Stroke, Atherosclerosis, Celiac disease, Deficiency of IL-1 Receptor Antagonist, Alzheimer's disease, Parkinson's disease, Multiple Sclerosis and Cancer.

40. The method of claim 38, wherein the IRAK4 mediated disease is selected from the group consisting from is selected from an autoimmune disease, an inflammatory disease, bone diseases, metabolic diseases, neurological and neurodegenerative diseases and/or disorders, cardiovascular diseases, allergies, asthma, hormone-related diseases, Ischemic stroke, Cerebral Ischemia, hypoxia, Traumatic Brain Injury, Chronic Traumatic Encephalopathy, epilepsy, Parkinson's disease, and Amyotrophic Lateral Sclerosis.
